(19) United States
(12) Patent Application Publication
(10) Pub. No.: US 2009/0269741 A1
(43) Pub. Date: Oct. 29, 2009

(54) Method for assessing traits selected from longissimus dorsi peak force, intramuscular fat, retail beef yield and net feed intake in bovine animals

(75) Inventors: William Barendse, Queensland (AU); Antonio Reverter-Gomez, Queensland (AU)

Correspondence Address: SUGHRUE MION, PLLC, 2100 PENNSYLVANIA AVENUE, N.W., SUITE 800, WASHINGTON, DC, 20037, US

(73) Assignees: MEAT & LIVESTOCK AUSTRALIA LIMITED, North Sydney (AU); COMMONWEALTH SCIENTIFIC AND INDUSTRIAL RESEARCH ORGANISATION, CAMPBELL (AU); DEPARTMENT OF PRIMARY INDUSTRIES FOR AND ON BEHALF OF THE STATE OF NEW SOUTH WALES, ORANGE (AU); TEH STATE OF QUEENSLAND THROUGH ITS DEPT OF PRIMARY INDUSTRIES AND FISHERIES, BRISBANE (AU)

(21) Appl. No.: 11/989,299

(22) PCT Filed: Jul. 26, 2006
(86) PCT No.: PCT/AU2006/001044

§ 371 Date: Dec. 29, 2008

(30) Foreign Application Priority Data
Jul. 26, 2005 (AU) 2005903955
Jul. 27, 2005 (AU) 2005903956
Jul. 28, 2005 (AU) 2005904041
Jul. 28, 2005 (AU) 2005904050
Aug. 16, 2005 (AU) 2005304422
Aug. 16, 2005 (AU) 2005904424
Aug. 16, 2005 (AU) 2005904425
Aug. 16, 2005 (AU) 2005904426
Aug. 26, 2005 (AU) 2005904658
Nov. 3, 2005 (AU) 2005906085
Nov. 3, 2005 (AU) 2005906086
Nov. 17, 2005 (AU) 2005906375
Nov. 17, 2005 (AU) 2005906376
Nov. 22, 2005 (AU) 2005906472
Nov. 22, 2005 (AU) 2005906473
Nov. 23, 2005 (AU) 2005906531
Nov. 23, 2005 (AU) 2005906532

(51) Int. Cl. C12Q 1/68; C07H 21/04

(52) U.S. Cl. 435/6; 536/24.31; 536/24.33

(57) ABSTRACT

A method for assessing a trait in a bovine animal selected from the group consisting of longissimus dorsi peak force, intramuscular fat, retail beef yield and net feed intake, comprising the steps of: (1) providing a nucleic acid from the bovine animal or carcass; (2) assaying for the occurrence of a single nucleotide polymorphism (SNP), wherein the identification of said nucleotide occurrence as set forth herein is associated with variation in longissimus dorsi peak force, intramuscular fat deposition, retail beef yield or net feed intake.

### TECHNICAL FIELD

The present invention is concerned with a method for assessing selected traits in bovine animals. In particular it is concerned with a method for assessing traits selected from (1) longissimus dorsi peak force (LDPF), which is also referred to as Warner-Bratzler shear force (WBS) and is a measure of meat tenderness in the longissimus dorsi muscle which is indicative of the characteristic known to consumers (and referred to herein) as “meat tenderness” (2) intramuscular fat deposition in animals, which in turn influences the characteristic of “marbling” in meat (3) retail beef yield (RBY) and (4) net feed intake (NFI). The invention is useful for the selection of animals which show desirable meat tenderness traits either for breeding or to select animals destined to be slaughtered for food; in the selection of animals for ability to produce high levels of marbling in meat; for the selection of animals which show desirable traits in RBY either for breeding or in feed lot processing; and in the selection of animals for efficiency of utilisation of feed with a view to producing cattle that eat less for the same liveweight gain.

### BACKGROUND ART

The characteristics of meat tenderness, intramuscular fat, retail beef yield and net feed intake are key characteristics of cattle which influence consumer demand and the economics of beef production.

Meat tenderness is an important issue for consumers, and one which can influence demand sufficiently for an especially tender meat to command a premium price in the marketplace. The physiological change in muscle structure during the post-mortem period is complex but clearly seems to be at least one factor in meat tenderness.

It has been theorised that an endogenous, calcium-dependent proteinase, calpain, initiates in vivo muscle protein degradation. In particular, calpain is believed to be responsible for the breakdown of myofibril protein, which is closely related to meat tenderness. The protein calpastatin regulates calpain activity. The action of lysyl oxidase is to initiate cross-link formation in collagen fibrillogenesis. Our International Publication No. 02/064820 describes a method for assessing tenderness in beef by testing for genetic markers in the gene encoding calpastatin (CAST) and/or the gene encoding lysyl oxidase (LOX).

Even so, there is no comprehensive system for improving meat tenderness using genetic markers.

The manner in which animals metabolise fat is of considerable economic significance in agriculture and animal husbandry. In some markets a high content of fat in meat, in the form of small fat deposits or “marbling”, is regarded as highly desirable, and so efforts are made to induce heavy marbling of meat in cattle. In particular, the animals may be grain fed for at least a short period prior to marketing and slaughter. In other markets a very lean meat is preferred. Thus there is a need for methods by which the propensity of bovine animals to deposit fat in muscle can be assessed.

Intramuscular or marbling fat is deposited in cattle between the fascicules of muscles, and usually develops when animals are fed a high calorie diet for a long time. The quantity of marbling fat is expressed either as a lipid concentration or as a standardised marbling score (eg. the Australian AUSMEAT standard). Unlike fat deposited in subcutaneous and renal depots, marbling fat is deposited continuously until relatively late in the development of the animal, and the amount of this fat is strongly correlated with the number of fat cells or adipocytes found in the muscle fascicules. Although some of the factors that are important in the differentiation of adipocytes are known, the genetic factors that are involved in the difference between individuals in differentiation and development of the interfascular adipocytes and deposition of fat were unknown until recently, as were the genetic variants leading to a high or low marbling score.

The genetic basis of lipid metabolism has been investigated, and our International Publication WO99/23248 describes a method for assessing lipid metabolism in bovine animals in which DNA markers associated with (a) the 5′ untranslated region of the gene encoding thyroglobulin (b) the gene encoding the retinoic acid receptor gamma (RARG) and (c) the gene encoding 11-cis,9-cis retinol dehydrogenase (RDH5) were employed. In particular, some markers were associated with the increased fat deposition in muscle tissue. Polymorphisms in the retinoid related orphan receptor C(gamma) (RORC) have also been found to be associated with increased fat deposition in muscle tissue, as described in our International Publication No. WO2004/070055.

Thus, although a number of DNA markers associated with lipid metabolism and marbling effects have been reported, there is no comprehensive system for improving marbling.

Retail beef yield is the amount of meat that can be obtained from a carcass and sold. Clearly not all of a carcass can be sold for meat, and a substantial amount of fat is cut off and rendered while little of the bone is included in retail cuts. The skin, hooves and horns enter the by-product chain immediately. So the carcass weight of the animal is partitioned and the retail yield attracts much greater prices than that which enters the chain associated with by-products. Because the amount of fat and bone vary significantly between animals at any weight, purchasing cattle on final weight is not efficient and methods of predicting the amount of retail beef yield that would be obtained from the carcass are currently used, although some methods are of limited accuracy.

Retail beef yield can be measured through exhaustive boning out of carcasses, which is time consuming and expensive, and breeding can only be done indirectly through reference to breeding values of sires, who cannot be measured directly. It can be estimated with varying degrees of accuracy, either through VIASCAN technology or through a comparison of P8 fat thickness and carcass weight. By their nature, this does not allow animals to be selected accurately at feedlot entry, and breeding using these methods requires strong feedback from processors to producers. Such feedback usually can occur in vertically integrated enterprises to enhance breeding.

There are no DNA markers currently implemented for retail beef yield.

It has long been a goal of world producers to select directly for feed efficiency. The trait is a key element of beef farming profitability, given that about 80% of feed resources in a cattle breeding enterprise are consumed by breeding females. Increased feed efficiency also relates to the sustainability of beef farming in areas where feed resources are scarce or pastures poor.

While the breeding objective is improvement of feed conversion efficiency, the trait used to measure the phenomenon is referred to as “net feed intake” (NFI). In simple terms, this is the amount of feed a cattle beast eats, under or over the amount predicted from the animal's size (weight) and growth rate. The more efficient cattle are those with a lesser net feed intake for the same weight gain i.e. the more efficient cattle eat less than the animal's size and growth rate would suggest.

Net feed intake is expensive and difficult to measure. It may cost up to $300 per animal, and there are limited accredited facilities where the test can be made. Individual feed intakes are currently measured over a set test period of seventy (70) days, during which time standard, medium energy hay and grain ration is offered. Test cattle are weighed regularly and their intakes compared with their growth performance to determine if they have eaten more or less than expected. NFI results are reported as kilograms of feed eaten per day, and generally expressed as an amount above or below the breed average. The more negative the number the more efficient is the animal (and its progeny) in feed utilisation.

Since net feed intake is moderately heritable, the trait can be improved by direct selection. In view of the difficulty of measuring net feed intake directly, a gene marker test for the trait would be desirable.

The use of a blood test for insulin-like growth factor (IGF-1) is described in the National Beef Recording Scheme BREEDNOTE 04/1—January 2004. IGF-1 is moderately heritable (0.4) and correlated to NFI (0.6 low IGF-1, more efficient). The test is currently applicable to Angus and Hereford herds, and it is expected that other breeds will follow as data becomes available. However, the test lacks precision and still requires that some cattle be measured for feed intake.

### SUMMARY OF THE INVENTION

The present inventor has identified a number of single nucleotide polymorphisms (SNP) which act as genetic markers for longissimus dorsi peak force (LDPF), which is indicative of meat tenderness, intramuscular fat deposition, which is indicative of marbling, retail beef yield and net feed intake characteristics. Thus the invention allows a rapid and precise test for genetic variation for the relevant trait, which would allow many cattle to be tested over a short period of time, in a cost effective manner, to establish these characteristics.

In a first aspect of the present invention there is provided a method for assessing a trait in a bovine animal selected from the group consisting of longissimus dorsi peak force, intramuscular fat, retail beef yield and net feed intake and/or its component traits, comprising the steps of:

(1) providing a nucleic acid from the bovine animal or carcass;

(2) assaying for the occurrence of a single nucleotide polymorphism (SNP) identified in any one of SEQ ID Nos: 1 to 1635, wherein the identification of said nucleotide occurrence as set forth (a) in any one of SEQ ID NOs: 1171 to 1631 is associated with variation in longissimus dorsi peak force, (b) in any of SEQ ID Nos: 214 to 842 is associated with intramuscular fat deposition, (c) in any one of SEQ ID NOs: 843 to 1170 is associated with retail beef yield and (d) in any one of SEQ ID NOs: 1 to 213 or 632 to 1635 is associated with net feed intake and/or its component traits.

It will be appreciated that the association of an allele or genotype with increased value will often apply across breeds and families within breeds. However, a particular allele or genotype may not always be associated with increased values across breeds; in one breed the allele or genotype might be associated with an increased value but in another breed it might be associated with decreased value or not be associated with difference in value. The person skilled in the art will be able to establish the direction of the association.

Advantageously the assay is a quantitative assay which is capable of determining the number of copies of each form of the SNP in the nucleic acid sample. In an embodiment the assay is a polymerase chain reaction (PCR) which employs unique primers designed to amplify the DNA molecules set forth in SEQ ID Nos: 1 to 1635 or a portion of these which contains the SNP, and complements thereof. However, other DNA based methods such as primer extension and oligonucleotide ligation assays could be used. Suitable methods for amplification of DNA of known sequence are well understood by the person skilled in the art, and application of such techniques is widely described, for example, in WO03/031592 (the contents of which are incorporated herein by reference).

According to a further aspect of the present invention there is provided a method for selecting a bovine animal within a population of bovine animals, comprising the steps of:

(1) providing a nucleic acid sample from the bovine animal;

(2) assaying for the occurrence of a single nucleotide polymorphism (SNP) identified in any one of SEQ ID Nos: 1 to 1635, wherein the identification of said nucleotide occurrence as set forth (a) in any one of SEQ ID NOs: 1171 to 1631 is associated with variation in longissimus dorsi peak force, (b) in any of SEQ ID Nos: 214 to 842 is associated with intramuscular fat deposition, (c) in any one of SEQ ID NOs: 843 to 1170 is associated with retail beef yield and (d) in any one of SEQ ID NOs: 1 to 213 or 1632 to 1635 is associated with net feed intake and/or its component traits; and

(3) selecting a bovine animal exhibiting the desired trait.

An animal selected on this basis may be sorted from the remaining population of bovine animals and managed differently in order to maximise the physical characteristic of the animal. The animal selected by this method may be selected for purposes of breeding from said animal, or a progenitor cell from an animal which exhibits this characteristic may be used in a method for cloning bovine animals.

The single nucleotide polymorphisms of the invention are set forth in the Tables which follow, and a sequence listing providing a description of the polymorphism and giving 3′ & 5′ flanking sequence has been filed in electronically. The correlation between the SEQ ID Nos and the polymorphisms of the invention are recited in the various tables. Therefore, in an embodiment the present invention involves detecting a part of any one of the nucleic acids as set forth in SEQ ID Nos: 1 to 1635.

According to a further aspect of the present invention there is provided a solid substrate or surface comprising a plurality of nucleic acids in separate physical locations, including at least one nucleic acid as set forth in SEQ ID NO: 1 to 1635, or fragments of at least 10 contiguous nucleotides which contain the polymorphism, immobilised thereon. For example, the nucleic acids of the present invention, or part of their sequence, may be used as a part or the whole of a microarray.

Additionally, primers or probes may be designed as described therein to hybridize to any one of SEQ ID NO: 1 to 1635 or a complementary sequence thereto.

According to a further aspect of the present invention there is provided a kit for assessing a trait in a bovine animal selected from the group consisting of longissimus dorsi peak force, intramuscular fat, retail beef yield and net feed intake and/or its component traits through detection of the occurrence of a single nucleotide polymorphism (SNP), wherein the identification of said nucleotide occurrence as set forth (a) in any one of SEQ ID NOs: 1171 to 1631 is associated with variation in longissimus dorsi peak force, (b) in any of SEQ ID Nos: 214 to 842 is associated with intramuscular fat deposition, (c) in any one of SEQ ID NOs: 843 to 1170 is associated with retail beef yield and (d) in any one of SEQ ID NOs: 1 to 213 or 1632 to 1635 is associated with net feed intake and/or its component traits, comprising an oligonucleotide probe, primer or primer pair, or combinations thereof, for determining the nucleotide occurrence of the SNP.

Advantageously the kit further comprises one or more detectable labels.

According to a further aspect of the present invention there is provided an oligonucleotide probe, primer or primer pair for detecting the occurrence of a single nucleotide polymorphism as set forth in any one of SEQ ID Nos: 1 to 1635.

According to a still further aspect of the invention there is provided a method for assessing a trait in a bovine animal selected from the group consisting of longissimus dorsi peak force, intramuscular fat, retail beef yield and net feed intake and/or its component traits, comprising the steps of:

(1) providing a nucleic acid from the bovine animal or carcass;

(2) assaying for the occurrence of a polymorphism in a gene, including in the coding sequences, the introns, promotors and other regulatory sequences of said gene, or a polymorphism in linkage disequilibrium with a polymorphism in said gene, wherein said gene is selected from the group consisting of CLCA4 to CLCA3, ABCC4, RPL11, TULP3, TRPC4, C6orf32, TPT1, GTF3C2, SLC6A15, CTNNA3, DDX46, HS2ST1, NDUFS3, PMS2, TMEM47, CEP1, CAP2, IMPG2, BAZ2B, SENP7, IMPG2, EFCBP2, DCP2, XBPP1, LAMA3, Synaptotagmin X (SYT 10), DMD, EMR2, ZNF33A, DNM1L, GBAS, SEC5L1, ATP1A1, YES1, BSN, POU4F1, SLIT3, ROBO1, KRT1-23, DDX10, GRM1 and BAAT, wherein the identification of said nucleotide occurrence (a) in any one of CLCA4 to CLCA3, ABCC4, RPL11, TULP3, TRPC4 and C6orf32 is associated with variation in longissimus dorsi peak force, (b) in any of TPT1, GTF3C2, SLC6A15, CTNNA3, DDX46, HS2ST1, NDUFS3, PMS2 and TMEM47 is associated with intramuscular fat deposition, (c) in any one of CEP1, CAP2, IMPG2, BAZ2B, SENP7, IMPG2, EFCBP2, DCP2, XBPP1 and LAMA3 is associated with retail beef yield and (d) in any one of Synaptotagmin X (SYT 10), DMD, EMR2, ZNF33A, DNM1L, GBAS, SEC5L1, ATP1A1, YES1, BSN, POU4F1, ROBO1, KRT1-23, DDX10 and BAAT is associated with net feed intake and/or its component traits.

According to a still further aspect of the invention there is provided a method for selecting a bovine animal within a population of bovine animals, comprising the steps of:

(1) providing a nucleic acid sample from the bovine animal;

(2) assaying for the occurrence of a polymorphism in a gene, including in the coding sequences, the introns, promotors and other regulatory sequences of said gene, or a polymorphism in linkage disequilibrium with a polymorphism in said gene, wherein said gene is selected from the group consisting of CLCA4 to CLCA3, ABCC4, RPL11, TULP3, TRPC4, C6orf32, TPT1, GTF3C2, SLC6A15, CTNNA3, DDX46, HS2ST1, NDUFS3, PMS2, TMEM47, CEP1, CAP2, IMPG2, BAZ2B, SENP7, IMPG2, EFCBP2, DCP2, XBPP1, LAMA3, Synaptotagmin X (SYT 10), DMD, EMR2, ZNF33A, DNM1L, GBAS, SEC5L1, ATP1A1, YES1, BSN, POU4F1, SLIT3, ROBO1, KRT1-23, DDX10, GRM1 and BAAT, and wherein the identification of said nucleotide occurrence (a) in any one of CLCA4 to CLCA3, ABCC4, RPL11, TULP3, TRPC4 and C6orf32 is associated with variation in longissimus dorsi peak force, (b) in any of TPT1, GTF3C2, SLC6A15, CTNNA3, DDX46, HS2ST1, NDUFS3, PMS2 and TMEM47 is associated with intramuscular fat deposition, (c) in any one of CEP1, CAP2, IMPG2, BAZ2B, SENP7, IMPG2, EFCBP2, DCP2, XBPP1 and LAMA3 is associated with retail beef yield and (d) in any one of Synaptotagmin X (SYT 10), DMD, EMR2, ZNF33A, DNM1L, GBAS, SEC5L1, ATP1A1, YES1, BSN, POU4F1, ROBO1, KRT1-23, DDX10 and BAAT is associated with net feed intake and/or its component traits; and

(3) selecting a bovine animal exhibiting enhancement of the desired trait.

According to a still further aspect of the invention there is provided a kit for assessing a trait in a bovine animal selected from the group consisting of longissimus dorsi peak force, intramuscular fat, retail beef yield and net feed intake and/or its component traits through detection of the occurrence of a single nucleotide polymorphism (SNP) in a gene, including in the coding sequences, the introns, promotors and other regulatory sequences of said gene, or a polymorphism in linkage disequilibrium with a polymorphism in said gene, wherein said gene is selected from the group consisting of CLCA4 to CLCA3, ABCC4, RPL11, TULP3, TRPC4, C6orf32, TPT1, GTF3C2, SLC6A15, CTNNA3, DDX46, HS2ST1, NDUFS3, PMS2, TMEM47, CEP1, CAP2, IMPG2, BAZ2B, SENP7, IMPG2, EFCBP2, DCP2, XBPP1, LAMA3, Synaptotagmin X (SYT 10), DMD, EMR2, ZNF33A, DNM1L, GBAS, SEC5L1, ATP1A1, YES1, BSN, POU4F1, SLIT3, ROBO1, KRT1-23, DDX10, GRM1 and BAAT, and wherein the identification of said nucleotide occurrence (a) in any one of CLCA4 to CLCA3, ABCC4, RPL11, TULP3, TRPC4 and C6orf32 is associated with variation in longissimus dorsi peak force, (b) in any of TPT1, GTF3C2, SLC6A15, CTNNA3, DDX46, HS2ST1, NDUFS3, PMS2 and TMEM47 is associated with intramuscular fat deposition, (c) in any one of CEP1, CAP2, IMPG2, BAZ2B, SENP7, IMPG2, EFCBP2, DCP2, XBPP1 and LAMA3 is associated with retail beef yield and (d) in any one of Synaptotagmin X (SYT 10), DMD, EMR2, ZNF33A, DNM1L, GBAS, SEC5L1, ATP1A1, YES1, BSN, POU4F1, ROBO1, KRT1-23, DDX10 and BAAT is associated with net feed intake and/or its component traits, comprising an oligonucleotide probe, primer or primer pair, or combinations thereof, for determining said nucleotide occurrence.

The invention is therefore also concerned, in further aspects, with animals when selected by the method of the invention, their progeny and the use of both selected animals and their progeny for breeding as well as meat from these animals.

The methods of the invention are applicable to bovine animals including but not limited to cattle, water buffalo and bison.

As used herein it will be understood that NFI is a composite phenotype measured as a function of three primary traits, namely (1) daily feed intake; (2) average daily gain during the feeding period; and (3) body weight (or typically metabolic mid-weight). This being stated, it is anticipated that some users of this technology will be interested in using the gene tests at the primary trait level. DNA markers for NFI will have greater or lesser impacts on the primary traits depending upon the DNA marker.

This specification contains nucleotide and amino acid sequence information prepared using PatentIn Version 3.3. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g., SEQ ID NO: 1 refers to the sequence in the sequence listing designated as <210>1 and the sequence information immediately follows the identifier <400>1). In the sequences the symbols Y, R, M, K, S and W have been used to indicate the polymorphism. Thus the symbol “M” represents an A/T polymorphism, and so on. In general the polymorphism occurs at position 201 or position 51, depending on whether 200 nucleotides of 50 nucleotides of flanking sequence have been added. The sequence flanking the polymorphism is derived from publicly available sequence information. The present invention is not restricted to detection of the entire nucleotide sequence or in any way restricted to use of the entire nucleotide sequence. This information is presented to assist in the design of oligonucleotide premiers and probes, but the person skilled in the art will recognize that such sequence may contain errors and will adjust their design accordingly.

The designation of nucleotide residues referred to herein are those recommended by the IUPAC-IUB Biochemical Nomenclature Commission, wherein A represents Adenine, C represents Cytosine, G represents Guanine, T represents Thymine, Y represents a pyrimidine residue, R presents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

Throughout this specification, unless specifically states otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein any range of numerals includes all within the range and the term “at least one” means one, two, three, four, etc. up to the possible maximum. Therefore a reference to one or more SNPs includes one SNP or any number of SNPs from two to the total number of SNPs set forth herein. The SNPs set forth herein can be used alone or in any combination, therefore the invention envisages detection of any of the possible combinations of SNPs set forth herein.

As used herein the term “cow” is used to refer to an individual animal without an intention to limit by gender and should not be taken to do so unless it is necessary from the context to infer that a female animal is referred to. The term should also be taken to encompass a young animal of either gender.

The present invention is not be limited in scope by the specific embodiments described herein, which are intended for the purpose of exemplification only. Functionally-equivalent products, compositions and methods are clearly within the scope of the invention, as described herein.

The present invention may be performed following the description herein without undue experimentation using, unless otherwise indicated, conventional techniques of molecular biology. Such procedures are described in various publications referred to throughout the specification, and the content of each such publication is incorporated herein by reference.

Throughout this specification and the claims, the words “comprise”, “comprises” and “comprising” are used in a non-exclusive sense, except where the context requires otherwise.

It will be clearly understood that, although a number of prior art publications are referred to herein, this reference does not constitute an admission that any of these documents forms part of the common general knowledge in the art, in Australia or in any other country.

Those skilled in the art will appreciate that the invention described herein is susceptible to variations and modifications other than those specifically described. It is to be understood that the invention includes all such variations and compounds referred to or indicated in this specification, individually or collectively, and any and all combinations or any two or more of said steps or features.

### DETAILED DESCRIPTION OF THE INVENTION

The methods of the invention allow for the management of bovine animals including selection of animals for breeding or cloning. The methods allow the identification of animals with favourable LDPF, intramuscular fat deposition, retail beef yield and/or net feed intake characteristics. Favourable characteristics may be present in certain animals singly or in combination, and measurement of these traits allows management of individual animals possessing one or more of these traits to maximise the individual potential performance. In particular, the methods of the invention allow management of feed intake, diet composition and diet related factors such as administration of food additives, feeding methods and management such as introduction of feed lots so that an individual animal may be treated in the most appropriate manner to produce meat of appropriate quality when slaughtered.

The principal commercial bovine animals are cattle, and there are many breeds of cattle. In an embodiment the cow is a breed (or cross) selected not exclusively from the group consisting of Angus, Ankole-Watusi, Ayrshire, Bazadaise, Beefalo, Beefmaster, Belgian Blue, Belmont Red, Blonde d'Aquitaine, Bonsmara, Braford, Brahman, Brahmousin, Brangus, Braunvieh, British White, American Brown Swiss, BueLingo, Charolais, Chianina, Corriente, American Devon, Dexters, Droughtmaster, Galloway, Gelbvieh, Guernsey, Hereford, Highland, Holstein, Jersey, Limousin, Lowline, Maine-Anjou, Marchigiana, Milking Shorthorn, Montebeliarde, Murray Grey, Normande, Parthenaise, Piedmontese, Pinzgauer, Romagnola, Salers, Salorn, Santa Gertrudis, Shetland, Shorthorn, Simmental, South Devon, Tarentaise, Texas Longhorn and Wagyu, most particularly Angus, Shorthorn, Hereford, Murray Grey, Brahman, Belmont Red and Santa Gertrudis.

Furthermore it will be appreciated that the association of an allele or genotype with increased value will often apply across breeds and families within breeds. However, a particular allele or genotype may not always be associated with increased values across breeds; in one breed the allele or genotype might be associated with an increased value but in another breed it might be associated with decreased value or not be associated with difference in value. The person skilled in the art will be able to establish the direction of the association.

Therefore, appropriate identification of traits in a particular species or breed allows for maximisation of physical characteristics of the animal to obtain meat with desirable tenderness and/or marbling characteristics and/or from animals with higher retail beef yield and/or low net feed intake and/or its component traits. Thus quality of the product and economics of its production may be adjusted either for an individual animal by management of that animal or through breeding and/or cloning of animals with desirable characteristics.

Single nucleotide polymorphisms are allelic variants that occur in a population where a single nucleotide difference is present at a locus. The method of the invention can involve detection of one single nucleotide polymorphism or more than one single nucleotide polymorphism, and can involve detection of single nucleotide polymorphisms which form or are a part of a haplotype which, as used herein, refers to groupings of two or more single nucleotide polymorphisms that are physically present in the same chromosome and tend to be inherited together except when recombination occurs. Methods for identifying haplotype alleles in nucleic acid samples are known to the person skilled in the art. This is from methods for haplotyping are described in WO 2005/040400, the contents of which are incorporated herein by reference.

A preferred sample for performing the method of the invention is a readily accessible sample that comprises genomic DNA. For example, genetic testing of cattle is often performed using a hair follicle, for example, isolated from the tail of an animal to be tested.

Other examples of readily accessible samples include, for example, bodily fluids or an extract thereof or a fraction thereof. For example, a readily accessible bodily fluid includes, for example, whole blood, saliva, semen or urine.

In another embodiment, a biological sample comprises a cell or cell extract or mixture thereof derived from a tissue such as, for example, skin.

Preferably, a biological sample has been isolated or derived previously from a subject by, for example, surgery, or using a syringe or swab.

Cell preparations or nucleic acid preparation derived from such tissues or cells are not to be excluded. The sample can be prepared on a solid matrix for histological analyses, or alternatively, in a suitable solution such as, for example, an extraction buffer or suspension buffer, and the present invention clearly extends to the testing of biological solutions thus prepared.

Analysis of the sample may be carried out by a number of methods. The present invention has identified a number of SNPs associated with traits in bovine animals, and subsequently detecting the presence or absence of the favourable allelic form of each SNP, or a plurality of these SNPs can be done using methods known in the art. Such methods may employ one or more oligonucleotide probes or primers including, for example, an amplification primer pair that selectively hybridize to a target polynucleotide which comprises a part or all of the sequence set forth in any one of SEQ ID Nos:1 to 1635. Oligonucleotide probes useful in an embodiment of the invention comprise an oligonucleotide which is complementary to and spans a portion of the polynucleotide including the SNP in question. Therefore, the presence of a specific nucleotide at the position (i.e. one of the allelic forms of the SNP) is detected by the ability or otherwise for the probe to hybridize. Such a method can further include contacting the target polynucleotide and hybridized oligonucleotide with an endonuclease and detecting the presence or absence of a cleavage product of the probe.

An oligonucleotide ligation assay also can be used to identify a nucleotide occurrence at a polymorphic position, wherein a pair of probes that selectively hybridize upstream and adjacent to and downstream and adjacent to the site of the SNP are prepared, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP loci can be sequenced using traditional sequence methodologies (e.g., the “dideoxy-mediated chain termination method,” also known as the “Sanger Method” (Sanger, F., et al. J. Molec. Biol. 94:441 (1975); Prober et al. Science 238:336-340 (1987)) and the “chemical degradation method,” also known as the “Maxam-Gilbert method” (Maxam, A. M., et al. Proc. Natl, Acad. Sci (U.S.A.) 74:560 (1977)), the contents of which are herein incorporated by reference to determine the nucleotide occurrence at the SNP loci.

As will be apparent to the person skilled in the art, the specific probe or primer used in an assay of the present invention will depend upon the assay format used. Methods of designing probes and/or primers for example, PCR or hybridization are known in the art and described, for example, in Dieffenbach and Dveksler (Eds) (In: PCR Primer: A Laboratory Manual, Cold Spring Harbour Laboratories, NY, 1995) and more recently in P.-Y. Kwok (Ed) (In: Methods in Molecular Biology Vol 212 Human Press, Totowa N.J., 2003). The various categories of polymorphism have been systematized and the various methods used to detect them have been thoroughly overviewed (BARENDSE and FRIES 1999). In short, there are only two kinds of polymorphism, those due to changes of DNA bases and those due to insertion and deletion of bases. Furthermore, the detection of these polymorphisms uses essentially the same technology and it is a rare technique that can be used for only one or the other of these kinds of polymorphism. Polymorphisms can also be divided into those that are in or near a sequence that is transcribed into RNA (type I polymorphisms) and those that are in DNA that is never translated into RNA (type II polymorphisms). However, all of these polymorphisms can be detected using the same kinds of methods. The methods for detecting DNA polymorphisms revolve around 3 major aspects, not all of which are used in every detection method, and some methods use techniques that are not one of these 3 major kinds. The point is that there are a large and ever increasing range of methods for detecting polymorphisms so it is not possible to be prescriptive about how the polymorphism should be detected, rather, it is the DNA sequence which lends itself to one or the other method of detection. Most of these methods are highly dependent upon the polymerase chain reaction (PCR), although it is possible to detect sequence differences easily without using the PCR. The three technologies are 1) separation of DNA by size or by composition, 2) oligonucleotide hybridisation to recognise specific DNA sequences, and 3) DNA visualisation. The DNA separation may be performed on solid matrices but may be performed in liquid matrices. The recognition of DNA sequence is usually performed by oligonucleotides of predefined sequence but may be performed enzymatically since some enzymes recognise specific DNA sequence motifs. DNA visualisation can be performed directly on the DNA which binds to some elements such as silver when it is visible in ordinary light, it may fluoresce under ultra violet light when it is bound to some molecules such as ethidium bromide, or it may be visualised through autoradiography when radioactive nucleotides are incorporated into the sequence. More usually, the DNA is visualised when it is bound to a DNA oligonucleotide which has a previously attached reporter molecule which may then be detected after laser excitation. Many methods depend on reporting the result of a specific reaction on the DNA, and may not even detect the DNA itself but remnants of the successful detection. These descriptions are merely to indicate the wide range and the many possible permutations of DNA detection, and do not exclude methods that have not been specifically referred to.

Some of the methods that might be used to detect the polymorphisms are described below, but they are not the only possible methods. While the specific hybridization of the probe or primer or other method for detecting variability to any nucleic acid can be predicted using well known rules, the probe or primer may not be unique if it is designed to bind to repetitive DNA sequence or to sequence common to members of a gene family, and so precautionary screening of probes and primers should be performed using, for example, BLAST against the cow and other genomes. In many cases this will not be sufficient and the adequacy of probes or primers may need to be confirmed empirically using methods known in the art. This same proviso will apply to all methods of detecting DNA that uses short probes and primers as would be appreciated by anyone skilled in the art.

The following is a list of some of the more useful current high throughput methods of detecting polymorphisms in cattle, but these should not be taken as an exhaustive list or be used to exclude new methods yet to be developed where they are essentially being used to identify the underlying DNA sequence. In that regard, if the DNA sequence has been transcribed to RNA and the RNA is tested for variation, or if the RNA has been translated to protein and the protein is tested for variation, these RNA and protein detection methods will also be methods of detecting the underlying DNA sequence. As indicated above, sometimes the detection method reports the result of a successful reaction without directly detecting the DNA molecule, and obviously this would apply for RNA and protein as well.

Some of the useful DNA based methods of detecting polymorphisms are the Taqman assay (LIVAK 2003), which uses competitive hybridisation of probes specific for the alternative DNA sequences and where a successful reaction is detected through the liberation of a reporter dye, the SNPlex assay (Applied Biosystems Incorporated, Foster City, Calif.) which uses the oligonucleotide ligation assay and where a successful reaction is reported via Zipchute probes that are separated on a capillary DNA sequencer, high throughput molecular inversion probes associated with generic microarray technologies (HARDENBOL et al. 2003; HARDENBOL et al. 2005), the MASSextend (STORM et al. 2002) or generic primer extension technologies (CHEN et al. 1997) which use mass spectrometry or laser fluorescence of the probe modified by an enzyme reaction respectively.

The present invention also relates to kits which can be used in the above described methods. Such kits typically contain an oligonucleotide probe, primer, or primer pair, or combinations thereof, depending upon the method to be employed. Such oligonucleotides are useful, for example, to identify a SNP as set forth herein. In addition, the kit may contain a control comprising oligonucleotides corresponding to the nucleotide sequence of the non-desired allelic form. In addition, the kit may contain reagents for performing a method of the invention such as buffers, detectable labels, one or more polymerases, which can be useful for a method that includes a primer extension or amplification procedure, and are nucleases for digesting hybridization products or a ligase which can be useful for performing an oligonucleotide ligation assay. The primers or probes can be included in the kit in labelled form.

The kit may also include instructions for use.

As well as management of the individual animal, the present invention allows for selection of animals for breeding programs. Thus a herd may be developed with desirable LDPF characteristics so as to have enhanced longissimus dorsi peak force, with desirable intramuscular fat deposition characteristics so as to increase the characteristic of marbling in meat, desirable beef yield in order to produce a herd with greater beef production characteristics and/or with reduced net feed intake to produce a herd with improved efficiency of freed use and therefore with favourable economic characteristics. As indicated previously, one or more of these traits may be selected for in the breeding program. Thus the method involves selected animals with desirable characteristics for one or more of these traits and using them in a breeding program. The progeny of the mating of selected parents are likely to contain the optimum combination of traits, thus creating a line of animals with specific characteristics. The progeny can then be used to breed and so on in order to continue the line, which may be monitored for purity using the original SNP markers.

Furthermore, the method of the invention allows for in vitro methods of producing animals. In general terms the method involves identification of one or more SNPs as set forth in SEQ ID Nos: 1 to 1635 in a bovine animal, isolating a progenitor cell from the animal and generating an animal from the progenitor cell. Methods of cloning bovine animals are well known to the person skilled in the art. For methods involved in cloning of cattle known methods may be used directly. As set forth, for example, in Willadsen “Cloning of sheep and cow embryos,” Genome 31:956 (1989), the contents of which are incorporated herein by reference.

In an embodiment, following development of an embryo, one or more cells is/are isolated therefrom and screened as described above. An embryo having or likely to have possess the desired trait or traits selected from longissimus dorsi peak force (LDPF), intramuscular fat deposition, retail beef yield (RBY) and net feed intake and/or its component traits (NFI) is then selected and implanted into a suitable recipient. In this manner, animals having or likely to have improved feeding efficiency are produced.

In an embodiment the selected animals are used to produce offspring using in vitro fertilization. In this process, ova are harvested from a cow comprising one or more SNP of the invention by, for example, transvaginal ovum pick-up (OPU) or by laparoscopic aspiration. The recovered ovum is then matured prior to fertilization. Zygotes are then cultured for a time and under conditions suitable for embryo development. For example, zygotes are cultured in a ligated oviduct of a temporary recipient (sheep or rabbit). Alternatively, zygotes are co-cultured in vitro with somatic cells (e.g., oviduct epithelial cells, granulosa cells, etc) in a defined medium. Alternatively, zygotes are cultured in vitro in a simple medium such as synthetic oviductal fluid without any somatic cell support.

The method is amenable to screening embryos produced using any assisted breeding technology and/or for screening embryos produced using an ovum and/or sperm from an animal that has not been screened using the method of the invention.

### EXAMPLE 1

### DNA Samples and Phenotypes

The net feed intake score for each animal was calculated. NFI is a predicted value based on a linear model and is not a raw measurement of the animal. All the animals were ranked from highest to lowest NFI and the top 200 and bottom 200 were extracted. For each of the herds of each of the seven breeds, an animal in the top 200 was matched to an animal in the bottom 200 (i.e. extremes) from the same herd with animals matched as best as possible to ensure that opposite animals represented a range of cohorts and market end points, and that high NFI was not systematically confounded with for example market or cohort when compared to animals of low NFI. After the DNA was quantified, some animals were found to have insufficient DNA to genotype 10,000 SNP, so those DNA samples were replaced by an animal with enough DNA that was part of the same herd but that was as close as possible to being in the opposite extreme 200 if an opposite extreme was not available. The final sample represents 41 Angus, 21 Brahman, 24 Belmont Red, 28 Hereford, 20 Murray Grey, 28 Santa Gertrudis and 27 Shorthorn animals. The 189 animals are 188 steers and 1 heifer and she was excluded from further analysis. These represent 142 sires with a range of 1 to 4 offspring per sire and a median of 1 offspring per sire. They represent 32 herds with a range of 1-12 animals per herd and a median of 5 steers per herd. They represent 37 kill groups with a range of 1-12 animals per kill group and a median of 4 per kill group. The contemporary group of each animal is its herd.kill_group.market.sex. The unadjusted NFI values for the sample range from −3.398 to 3.805 with a mean of 0.07 and standard deviation of 1.32. The total sample from which this sample was drawn had a mean of 0.00 and standard deviation of 0.75, showing that using the extremes increased the variability of the sample that was analysed. The mean of the one NFI extreme was −1.06 with a standard deviation of 1.63 (N=95) and the mean of the other NFI extreme was 1.21 with a standard deviation of 0.90 (N=94).

DNA was extracted from blood using Qiagen columns following the manufacturer's instructions. The DNA was quantified using fluorescence after the pico green dye was added to a small sample. The DNA was also quantified using UV spectrophotometry and the purity determined using the ratio of fluorescence at 260 versus 280 nm.

The samples were genotyped using the ParAllele 10K standard SNP panel. The method of SNP genotyping is documented in Hardenbol, P., Baner, J., Jain, M., Nilsson, M., Namsaraev, E. A., Karlin-Neumann, G. A., Fakhrai-Rad, H., Ronaghi, M., Willis, T. D., Landegren, U. and Davis, R. W. 2003 “Muliplex genotyping with sequence-tagged molecular inversion probes” Nature Biotechnology 21, 673-678 (2003), the contents of which are incorporated herein by reference. The panel of approximately 10,000 bovine SNP was derived from the publicly available DNA sequence and is called the MegAllele Genotyping Bovine 10K SNP Panel. The SNPs allow researchers to perform linkage mapping studies on bovine breeds with no bias towards either beef or dairy. The MegAllele Genotyping Bovine 10K SNP Panel is designed to work with the Affymetrix GeneChip Scanner 3000.

In the Examples that follow the person skilled in the art can determine from the Tables contained in this specification which genotype or allele is superior for any of traits listed. The genotypes have been coded in a consistent scheme so that this information could be retrieved irrespective of the actual bases at the single nucleotide polymorphism. Genotypes were coded as 0, 1, 2 and 5 where 5 is unknown, 1 is always the heterozygote, 0 is the homozygote higher up the alphabet and 2 is the homozygote lower down the alphabet—so CC is 2 when AA is the alternative homozygote, so the genotypes for an A/C SNP, with genotypes AA, AC and CC, are coded as 0, 1 and 2 respectively. For a C/G SNP, with genotypes CC, CG and GG the homozygote CC is now coded as 0, the CG heterozygote as 1 and the GG homozygote as 2.

In the Tables showing the effect of each SNP, the mean values for each genotype is given as mean_{—}0 for genotype 0, mean_{—}1 for genotype 1, and mean_{—}2 for genotype 2. These show clearly the performance of each genotype. In addition, to make the information more digestible, the additive effect a of each SNP, its dominance deviation k, and alpha the average effect of allele substitution are also given. A minus value for a means the mean of genotype 0 is lower than the mean of genotype 2, and a minus value for alpha means that selecting for allele 0 will reduce the average values of the trait in the population. By comparing a and alpha for each SNP identifier, and by looking at the DNA sequence of the SNP, if, for example, the alpha is negative, and the SNP is, for example, a C/T SNP, that would mean that the C allele reduces the trait value in the population. If on the other hand, the alpha is positive, and the SNP is, for example a G/T SNP, that would mean that the T allele increases the trait value in the population.

### EXAMPLE 2

### Analysis of the Associations Between NFI and DNA Markers

Net feed intake is a commercially import trait of cattle that represents the efficiency with which cattle or other species can use feed. We genotyped divergent pairs of animals of extreme net feed intake, a few divergent pairs from each sire, spread across a range of contemporary groups for 7 breeds, Angus, Shorthorn, Hereford, Murray Grey, Brahman, Belmont Red and Santa Gertrudis or described in Example 1. We genotyped these animals for more than 9,200 single nucleotide polymorphisms (SNP) across the bovine genome spread at less than 350 kb average spacing using the ParAllele genotyping system (Table 1). We found initially 9 SNP with very strong statistical significance and approximately 80 SNP with statistical significance at a=0.01 threshold, as set out in Tables 2 and 3, respectively.

Since the data have many contemporary groups, breeds, cohorts and markets, the phenotypes were first analysed to determine if there were any systematic differences between these. The linear model:

Nfi=mean+line+breed(line)+market+line*market+error

was found to explain 20% of the variation. In line with this model, the least squares mean NFI values for each breed*line*market combination was calculated and the NFI of each animal was adjusted accordingly. The adjusted average NFI value of each genotype was calculated and at each locus, the alternative genotypic means with the largest difference was compared using at test. To determine the significance of the t test, 100,000 permutations of genotypes at each locus was calculated and the proportion that gave a larger t test was calculated. This proportion is a distribution free P value for each comparison.

TABLE 1
Associations between DNA markers and net feed intake sorted in decreasing order of statistical significance.

| Locus | Scaffold-v2 | bp | IBISS4/Scaffold-v1 | mean_0 | SD | mean_1 | SD | mean_2 |
|---|---|---|---|---|---|---|---|---|
| 343617 | Un.1536 | 47639 | SCAFFOLD35407_3401 | −0.09 | 1.75 | −0.22 | 1.78 | 1.95 |
| 345016 | Un.119 | 491520 | SCAFFOLD291452_7280 | −0.32 | 1.81 | −0.10 | 1.69 | 1.14 |
| 349388 | 16.105 | 599843 | SCAFFOLD261939_1026 | 0.14 | 1.84 | 0.29 | 1.46 | −2.51 |
| 346973 | 13.119 | 223176 | SCAFFOLD71532_708 | −0.78 | 1.53 | 0.64 | 1.42 | 0.20 |
| 353336 | Un.1206 | 229702 | SCAFFOLD352562_535 | 2.20 | 1.69 | 0.12 | 1.63 | −0.15 |
| 350549 | 22.79 | 313784 | SCAFFOLD85350_16238 | 1.14 | 1.80 | 0.27 | 1.77 | −0.58 |
| 347269 | 6.14 | 2529 | SCAFFOLD165450_8470 | −1.93 | 0.79 | 0.26 | 1.83 | 0.18 |
| 350155 | Un.1886 | 35221 | SCAFFOLD240520_5556 | 0.52 | 1.77 | −0.52 | 1.69 | 0.93 |
| 342650 | Un.1230 | 70874 | IBISS4snp540 | −0.03 | 1.82 | 0.30 | 1.70 | 2.37 |
| 347570 | Un.8737 | 23429 | SCAFFOLD260891_2797 | 1.07 | 1.79 | 0.01 | 1.67 | −0.29 |
| 351382 | Un.1849 | 124957 | SCAFFOLD115666_13006 | 0.12 | 1.73 | 0.46 | 1.87 | −1.76 |
| 345385 | Un.779 | 60958 | SCAFFOLD11677_746 | 0.00 | 1.79 | 0.72 | 1.86 | −0.47 |
| 352721 | Un.1579 | 208904 | SCAFFOLD216584_13710 | 0.04 | 1.82 | 0.68 | 1.62 | −2.00 |
| 351385 | 2.38 | 115438 | SCAFFOLD116234_3345 | 0.18 | 1.79 | −1.73 | 0.68 | 0.00 |
| 346924 | Un.1139 | 232148 | SCAFFOLD55122_7765 | 0.38 | 1.91 | 0.07 | 1.70 | −0.97 |
| 349252 | Un.1579 | 209055 | SCAFFOLD216584_13861 | 0.07 | 1.85 | 0.52 | 1.49 | −2.00 |
| 342920 | Un.1230 | 53481 | SCAFFOLD191247_3964 | 0.00 | 1.80 | 0.33 | 1.74 | 2.41 |
| 354212 | Un.1139 | 232366 | SCAFFOLD55122_7983 | 0.38 | 1.91 | 0.09 | 1.68 | −0.97 |
| 352371 | NULL | NULL | SCAFFOLD321814_14337 | 0.13 | 1.87 | 0.38 | 1.71 | −1.05 |
| 348465 | Un.284 | 1868 | SCAFFOLD2481_1870 | 0.09 | 1.76 | 0.57 | 1.97 | −1.96 |
| 342844 | Un.16 | 119868 | SCAFFOLD135269_17123 | 0.11 | 1.84 | 1.08 | 1.54 | −0.74 |
| 344428 | 12.82 | 302857 | SCAFFOLD86623_4947 | 0.27 | 1.82 | 0.25 | 1.85 | −0.95 |
| 345597 | Un.5189 | 28900 | SCAFFOLD193911_3423 | 0.00 | 0.00 | −0.91 | 1.54 | 0.25 |
| 348073 | Un.8037 | 17164 | SCAFFOLD110844_5365 | 0.00 | 0.00 | −1.01 | 1.69 | 0.26 |
| 353131 | Un.1610 | 62295 | SCAFFOLD171003_4691 | 0.55 | 1.95 | 0.05 | 1.65 | −0.53 |
| 350185 | 23.81 | 534485 | SCAFFOLD255402_26573 | 0.18 | 1.69 | −0.34 | 1.90 | 1.71 |
| 351885 | 20.56 | 298267 | SCAFFOLD6887_2230 | 0.22 | 1.83 | 0.16 | 1.68 | −1.42 |
| 352772 | 1.146 | 36918 | SCAFFOLD25159_19124 | −0.65 | 1.25 | 0.50 | 1.85 | −0.03 |
| 348908 | 6.1 | 269406 | SCAFFOLD10121_13105 | 0.03 | 1.82 | 1.24 | 0.82 | 0.00 |
| 348351 | Un.4426 | 43261 | SCAFFOLD20608_1008 | −0.94 | 1.09 | 0.51 | 1.88 | 0.05 |
| 351082 | Un.1038 | 100048 | SCAFFOLD301148_20414 | −0.55 | 1.52 | 1.98 | 0.71 | 0.15 |
| 347501 | 12.83 | 29538 | SCAFFOLD245021_7420 | −0.14 | 1.73 | 0.02 | 1.77 | 1.28 |
| 348678 | Un.310 | 515663 | SCAFFOLD325650_1207 | 0.03 | 1.82 | 0.93 | 1.57 | −1.34 |
| 350023 | Un.1100 | 235321 | SCAFFOLD190318_15654 | 0.00 | 0.00 | −0.94 | 1.37 | 0.23 |
| 352237 | 13.16 | 119108 | SCAFFOLD250157_11623 | −1.20 | 1.46 | 0.61 | 1.72 | −0.06 |
| 344238 | 10.82 | 1271166 | SCAFFOLD295006_45304 | −0.19 | 1.66 | 0.18 | 1.84 | 1.28 |
| 349640 | Un.3198 | 77113 | SCAFFOLD55648_3312 | −1.68 | 1.35 | 0.39 | 1.45 | 0.16 |
| 345533 | Un.4542 | 9983 | SCAFFOLD170036_23917 | −0.32 | 1.39 | 1.02 | 1.44 | 0.01 |
| 344822 | Un.11087 | 3391 | SCAFFOLD220929_12275 | 0.00 | 0.00 | 1.13 | 1.85 | −0.07 |
| 347653 | Un.1101 | 174909 | SCAFFOLD290984_3799 | 0.20 | 1.81 | −0.22 | 1.72 | −2.03 |
| 346024 | 2.18 | 667311 | SCAFFOLD49069_4838 | 0.04 | 1.83 | 0.71 | 1.60 | −0.51 |
| 354433 | 6.154 | 53376 | SCAFFOLD205065_7657 | 0.15 | 0.98 | −1.88 | 1.22 | 0.23 |
| 351039 | Un.323 | 78544 | SCAFFOLD285539_13746 | 2.10 | 0.10 | 0.18 | 1.96 | −0.01 |
| 346800 | Un.1661 | 15087 | SCAFFOLD305898_10192 | 0.25 | 1.79 | −0.95 | 1.55 | 0.00 |
| 346873 | 11.173 | 210285 | SCAFFOLD38607_6519 | −0.76 | 1.60 | 0.64 | 2.08 | 0.17 |
| 344576 | 3.162 | 380554 | SCAFFOLD115003_498 | −1.24 | 1.54 | 0.13 | 1.82 | 0.30 |
| 352302 | 6.71 | 1672 | SCAFFOLD284472_542 | 0.26 | 1.77 | 0.16 | 1.87 | −1.10 |
| 350934 | 4.134 | 1432811 | SCAFFOLD245150_4016 | 1.02 | 1.66 | −0.05 | 1.68 | −0.12 |
| 352079 | 7.78 | 485886 | SCAFFOLD15666_3538 | 0.25 | 1.86 | 0.72 | 1.93 | −0.42 |
| 343193 | 22.79 | 298472 | IBISS4snp651 | −0.51 | 1.53 | −0.05 | 1.85 | 0.58 |
| 347999 | Un.10660 | 8528 | IBISS4snp723 | 0.13 | 1.88 | −0.21 | 1.81 | 1.55 |
| 351114 | Un.71 | 171881 | SCAFFOLD317129_13923 | −0.06 | 1.67 | −0.24 | 1.75 | 0.93 |
| 344483 | 19.45 | 624431 | IBISS4snp347 | −0.50 | 1.72 | 0.67 | 1.82 | −0.02 |
| 349908 | 20.1 | 343146 | SCAFFOLD145774_16171 | 0.23 | 2.05 | 0.51 | 1.65 | −0.59 |
| 347029 | Un.9514 | 2997 | SCAFFOLD91431_3586 | 0.60 | 1.75 | 0.05 | 1.85 | −0.52 |
| 343763 | 1.1 | 1178440 | IBISS4snp160 | 0.73 | 1.77 | 0.11 | 1.81 | −0.34 |
| 351837 | 20.7 | 11561 | SCAFFOLD48045_564 | 2.05 | 1.42 | 0.18 | 1.79 | −0.10 |
| 346220 | Un.5571 | 12011 | SCAFFOLD100297_8761 | 0.00 | 0.00 | −1.47 | 1.06 | 0.18 |
| 343692 | 3.216 | 275639 | SCAFFOLD71577_11565 | −0.19 | 1.59 | 0.65 | 2.00 | −0.41 |
| 347727 | Un.5716 | 44061 | SCAFFOLD316858_23449 | −0.44 | 1.58 | 0.79 | 1.80 | 0.26 |
| 354067 | 9.37 | 404806 | SCAFFOLD321782_15219 | 1.06 | 1.83 | 0.00 | 0.00 | −0.06 |
| 350815 | Un.2354 | 93446 | SCAFFOLD185691_4123 | 0.15 | 1.83 | 0.41 | 1.85 | −0.61 |
| 348912 | Un.51 | 620188 | SCAFFOLD10204_22524 | 0.04 | 1.80 | 0.88 | 1.57 | −0.93 |
| 351157 | 6.133 | 323039 | SCAFFOLD40038_18553 | −2.01 | 0.42 | 0.40 | 1.75 | 0.07 |
| 350542 | Un.3710 | 57861 | SCAFFOLD85037_17745 | −1.90 | 0.53 | −0.27 | 1.73 | 0.24 |
| 343638 | 16.35 | 81505 | SCAFFOLD466_4857 | 0.84 | 1.64 | −0.22 | 1.94 | 0.04 |
| 351119 | 22.29 | 253978 | SCAFFOLD317870_2261 | −0.78 | 1.37 | 0.44 | 1.94 | 0.07 |
| 344670 | 14.31 | 47171 | SCAFFOLD155146_6104 | 0.19 | 1.67 | 0.45 | 1.94 | −0.57 |
| 352540 | Un.16478 | 4033 | SCAFFOLD113475_827 | 0.22 | 1.76 | −1.31 | 1.74 | 0.00 |
| 343870 | Un. 12834 | 3514 | SCAFFOLD122517_7619 | −0.60 | 1.72 | 0.31 | 1.76 | 0.44 |
| 345255 | Un.9514 | 3234 | SCAFFOLD91431_3349 | 0.60 | 1.81 | 0.03 | 1.79 | −0.54 |
| 351524 | Un.304 | 246845 | SCAFFOLD200011_13411 | −0.08 | 1.77 | 0.83 | 1.76 | 0.00 |
| 342878 | Un.465 | 523854 | SCAFFOLD155037_16984 | 0.31 | 1.75 | −0.55 | 1.72 | 0.00 |
| 348581 | 26.54 | 208100 | SCAFFOLD288338_3076 | 0.13 | 1.82 | −1.19 | 0.88 | 0.00 |
| 343301 | Un.9948 | 10551 | SCAFFOLD15017_7532 | 1.04 | 1.86 | −0.16 | 1.68 | 0.01 |
| 350386 | Un.2683 | 58065 | SCAFFOLD523674_1755 | −0.70 | 1.59 | 0.38 | 1.75 | 0.18 |
| 347069 | Un.4773 | 40061 | IBISS4snp426 | 0.24 | 1.79 | 0.20 | 1.80 | −1.05 |
| 354228 | Un.2727 | 71680 | SCAFFOLD75727_10291 | 0.16 | 1.84 | 0.68 | 1.64 | −0.46 |
| 353758 | Un.3746 | 22662 | SCAFFOLD130522_9733 | 0.12 | 1.83 | −0.23 | 1.87 | 0.91 |
| 351068 | Un.10812 | 19943 | SCAFFOLD295225_17655 | 0.00 | 0.00 | 0.91 | 1.67 | −0.07 |
| 348529 | 24.79 | 5587 | SCAFFOLD268943_3771 | −0.31 | 1.60 | −0.04 | 1.77 | 0.83 |
| 348917 | Un.3423 | 55305 | SCAFFOLD105096_24896 | 0.76 | 1.42 | −0.43 | 1.45 | 0.14 |
| 351930 | 17.51 | 218776 | SCAFFOLD95294_4333 | 1.23 | 1.94 | −0.09 | 1.75 | −0.14 |
| 354405 | 14.104 | 194338 | SCAFFOLD90324_3708 | 0.12 | 1.93 | 0.28 | 1.59 | −1.11 |
| 353697 | Un.2727 | 71795 | SCAFFOLD75727_10176 | 0.21 | 1.97 | 0.57 | 1.65 | −0.44 |

| Locus | SD | N | Freq | a | k | alpha | tmax | log(1/P) |
|---|---|---|---|---|---|---|---|---|
| 343617 | 1.40 | 150 | 0.72 | −1.02 | 1.13 | −1.53 | 8.193 | 5.0000 |
| 345016 | 1.62 | 169 | 0.57 | −0.73 | 0.70 | −0.80 | 6.250 | 4.0000 |
| 349388 | 0.46 | 170 | 0.89 | 1.32 | 1.11 | 2.47 | 12.560 | 3.6380 |
| 346973 | 1.95 | 162 | 0.30 | −0.49 | −1.90 | −0.86 | 5.926 | 3.4810 |
| 353336 | 1.72 | 170 | 0.20 | 1.18 | −0.77 | 1.71 | 7.326 | 3.3870 |
| 350549 | 1.78 | 136 | 0.44 | 0.86 | −0.01 | 0.86 | 5.975 | 3.3190 |
| 347269 | 1.76 | 170 | 0.19 | −1.05 | −1.08 | −1.76 | 9.065 | 3.2680 |
| 350155 | 1.62 | 169 | 0.61 | −0.21 | 6.07 | −0.48 | 5.848 | 3.1190 |
| 342650 | 0.53 | 166 | 0.86 | −1.20 | 0.73 | −1.82 | 10.398 | 3.0360 |
| 347570 | 1.79 | 169 | 0.40 | 0.68 | −0.56 | 0.76 | 5.464 | 3.0270 |
| 351382 | 1.15 | 161 | 0.80 | 0.94 | 1.36 | 1.71 | 7.703 | 2.9390 |
| 345385 | 1.43 | 168 | 0.61 | 0.23 | 4.06 | 0.45 | 5.156 | 2.8630 |
| 352721 | 0.58 | 168 | 0.88 | 1.02 | 1.63 | 2.28 | 10.619 | 2.8360 |
| 351385 | 0.00 | 170 | 0.98 | 0.09 | −20.22 | −1.66 | 9.224 | 2.7400 |
| 346924 | 1.38 | 167 | 0.68 | 0.68 | 0.54 | 0.80 | 5.456 | 2.6970 |
| 349252 | 0.58 | 169 | 0.88 | 1.03 | 1.43 | 2.17 | 10.343 | 2.6930 |
| 342920 | 0.19 | 169 | 0.88 | −1.21 | 0.73 | −1.87 | 11.746 | 2.6740 |
| 354212 | 1.38 | 170 | 0.67 | 0.68 | 0.57 | 0.81 | 5.500 | 2.6560 |
| 352371 | 1.32 | 169 | 0.71 | 0.59 | 1.42 | 0.95 | 5.753 | 2.6460 |
| 348465 | 0.21 | 170 | 0.92 | 1.02 | 1.47 | 2.28 | 9.250 | 2.6360 |
| 342844 | 1.09 | 164 | 0.88 | 0.42 | 3.28 | 1.50 | 5.819 | 2.5590 |
| 344428 | 1.22 | 169 | 0.71 | 0.61 | 0.97 | 0.86 | 5.188 | 2.4930 |
| 345597 | 1.78 | 169 | 0.08 | −0.12 | 8.28 | 0.74 | 4.656 | 2.4830 |
| 348073 | 1.76 | 170 | 0.06 | −0.13 | 8.77 | 0.86 | 4.630 | 2.4720 |
| 353131 | 1.56 | 168 | 0.57 | 0.54 | 0.07 | 0.55 | 4.697 | 2.4440 |
| 350185 | 1.10 | 169 | 0.79 | −0.77 | 1.68 | −1.50 | 7.210 | 2.4340 |
| 351885 | 1.19 | 170 | 0.81 | 0.82 | 0.93 | 1.30 | 5.987 | 2.4200 |
| 352772 | 1.83 | 169 | 0.35 | −0.31 | −2.71 | −0.56 | 5.029 | 2.4150 |
| 348908 | 0.00 | 170 | 0.97 | 0.01 | 81.67 | 1.17 | 5.742 | 2.4090 |
| 348351 | 1.79 | 170 | 0.21 | −0.49 | −1.93 | −1.05 | 5.681 | 2.4030 |
| 351082 | 1.83 | 166 | 0.14 | −0.35 | −6.23 | −1.93 | 8.609 | 2.3820 |
| 347501 | 1.78 | 170 | 0.68 | −0.71 | 0.77 | −0.90 | 5.090 | 2.3790 |
| 348678 | 0.17 | 163 | 0.93 | 0.69 | 2.31 | 2.04 | 9.543 | 2.3750 |
| 350023 | 1.82 | 169 | 0.06 | −0.12 | 9.17 | 0.82 | 4.640 | 2.3360 |
| 352237 | 1.79 | 170 | 0.24 | −0.57 | −2.18 | −1.22 | 5.982 | 2.3310 |
| 344238 | 1.85 | 169 | 0.71 | −0.73 | 0.50 | −0.89 | 4.995 | 2.3200 |
| 349640 | 1.84 | 168 | 0.14 | −0.92 | −1.25 | −1.74 | 6.354 | 2.3000 |
| 345533 | 1.87 | 154 | 0.20 | −0.17 | −7.12 | −0.86 | 4.973 | 2.2880 |
| 344822 | 1.74 | 169 | 0.09 | 0.04 | 33.29 | −0.93 | 4.260 | 2.2700 |
| 347653 | 0.23 | 169 | 0.90 | 1.11 | 0.62 | 1.67 | 10.070 | 2.2550 |
| 346024 | 1.72 | 170 | 0.72 | 0.28 | 3.44 | 0.70 | 4.686 | 2.2540 |
| 354433 | 1.81 | 160 | 0.07 | −0.04 | 51.75 | 1.76 | 6.174 | 2.2320 |
| 351039 | 1.71 | 169 | 0.18 | 1.05 | −0.82 | 1.60 | 11.656 | 2.2320 |
| 346800 | 0.00 | 168 | 0.94 | 0.12 | −8.60 | −0.83 | 4.412 | 2.2290 |
| 346873 | 1.69 | 153 | 0.27 | −0.47 | −2.01 | −0.89 | 4.769 | 2.2280 |
| 344576 | 1.74 | 170 | 0.29 | −0.77 | −0.78 | −1.02 | 5.330 | 2.2280 |
| 352302 | 1.35 | 169 | 0.72 | 0.68 | 0.85 | 0.94 | 5.130 | 2.1990 |
| 350934 | 1.83 | 170 | 0.32 | 0.57 | −0.88 | 0.75 | 4.597 | 2.1870 |
| 352079 | 1.47 | 159 | 0.55 | 0.33 | 2.40 | 0.42 | 4.478 | 2.1810 |
| 343193 | 1.77 | 168 | 0.40 | −0.54 | 0.16 | −0.53 | 4.566 | 2.1770 |
| 347999 | 0.55 | 153 | 0.85 | −0.71 | 1.48 | −1.44 | 7.289 | 2.1670 |
| 351114 | 1.97 | 163 | 0.62 | −0.49 | 1.36 | −0.66 | 4.478 | 2.1660 |
| 344483 | 1.74 | 170 | 0.32 | −0.24 | −3.88 | −0.57 | 4.560 | 2.1590 |
| 349908 | 1.57 | 169 | 0.46 | 0.41 | 1.68 | 0.35 | 4.424 | 2.1550 |
| 347029 | 1.64 | 163 | 0.55 | 0.56 | 0.02 | 0.56 | 4.479 | 2.1540 |
| 343763 | 1.70 | 168 | 0.46 | 0.54 | −0.16 | 0.54 | 4.404 | 2.1350 |
| 351837 | 1.75 | 170 | 0.23 | 1.07 | −0.74 | 1.51 | 6.433 | 2.1310 |
| 346220 | 1.82 | 158 | 0.04 | −0.09 | 17.33 | 1.35 | 5.786 | 2.1310 |
| 343692 | 1.52 | 169 | 0.62 | 0.11 | 8.64 | 0.33 | 4.500 | 2.1250 |
| 347727 | 1.93 | 167 | 0.66 | −0.35 | −2.51 | −0.07 | 4.528 | 2.1190 |
| 354067 | 1.76 | 156 | 0.17 | 0.56 | −0.89 | 0.89 | 4.036 | 2.1100 |
| 350815 | 1.45 | 170 | 0.56 | 0.38 | 1.68 | 0.46 | 4.408 | 2.1090 |
| 348912 | 1.77 | 168 | 0.86 | 0.49 | 2.73 | 1.43 | 5.013 | 2.0970 |
| 351157 | 1.81 | 168 | 0.14 | −1.04 | −1.32 | −2.03 | 10.490 | 2.0960 |
| 350542 | 1.80 | 170 | 0.11 | −1.07 | −0.52 | −1.51 | 8.347 | 2.0910 |
| 343638 | 1.64 | 164 | 0.41 | 0.40 | −1.65 | 0.52 | 4.368 | 2.0810 |
| 351119 | 1.74 | 169 | 0.29 | −0.43 | −1.87 | −0.76 | 4.782 | 2.0800 |
| 344670 | 1.62 | 169 | 0.56 | 0.38 | 1.68 | 0.46 | 4.336 | 2.0800 |
| 352540 | 0.00 | 170 | 0.95 | 0.11 | −12.91 | −1.18 | 4.452 | 2.0770 |
| 343870 | 1.81 | 160 | 0.45 | −0.52 | −0.75 | −0.56 | 4.292 | 2.0760 |
| 345255 | 1.66 | 165 | 0.54 | 0.57 | 0.00 | 0.57 | 4.429 | 2.0590 |
| 351524 | 0.00 | 170 | 0.88 | −0.04 | −21.75 | 0.63 | 3.858 | 2.0580 |
| 342878 | 0.00 | 153 | 0.87 | 0.15 | −4.55 | −0.37 | 3.829 | 2.0530 |
| 348581 | 0.00 | 166 | 0.97 | 0.07 | −19.31 | −1.11 | 5.457 | 2.0470 |
| 343301 | 1.79 | 169 | 0.34 | 0.52 | −1.33 | 0.73 | 4.478 | 2.0420 |
| 350386 | 1.90 | 169 | 0.48 | −0.44 | −1.45 | −0.47 | 4.363 | 2.0400 |
| 347069 | 1.47 | 170 | 0.74 | 0.65 | 0.94 | 0.94 | 4.789 | 2.0400 |
| 354228 | 1.66 | 157 | 0.67 | 0.31 | 2.68 | 0.58 | 4.388 | 2.0390 |
| 353758 | 1.18 | 165 | 0.73 | −0.40 | 1.89 | −0.74 | 4.678 | 2.0380 |
| 351068 | 1.79 | 170 | 0.09 | 0.04 | 27.00 | −0.74 | 3.924 | 2.0360 |
| 348529 | 2.02 | 157 | 0.48 | −0.57 | 0.53 | −0.56 | 4.341 | 2.0340 |
| 348917 | 1.90 | 170 | 0.21 | 0.31 | −2.84 | 0.83 | 4.614 | 2.0220 |
| 351930 | 1.68 | 162 | 0.30 | 0.69 | −0.93 | 0.94 | 4.574 | 2.0060 |
| 354405 | 1.19 | 170 | 0.76 | 0.61 | 1.26 | 1.02 | 5.281 | 2.0010 |
| 353697 | 1.69 | 169 | 0.45 | 0.33 | 2.11 | 0.26 | 4.200 | 2.0010 |

Locus is the ParAllele identifiers of the polymorphisms, N is the number of genotypes, Scaffold-v2 is the Draft 2 scaffold of the bovine genome sequence, bp is the base pair within the scaffold, IBISS4 is the IBISS4 database identifier, Scaffold-v1 is the draft 1 scaffold of the bovine genome sequence, mean_0 is the mean net feed intake for genotype 0, mean_1 is the mean for genotype 1, and mean_2 is the mean of genotype 2, SD is the standard deviation, a is the additive effect, k is the dominance effect, alpha is the average effect of allele substitutions, tmax is the value of the t test, and log(1/P) is the P value determined from 100,000 permutation tests expressed as a positive integer. A minus value for a means the mean of genotype 0 is lower than the mean of genotype 2, and a minus value for alpha means that selecting for allele 0 will reduce the average values of net feed intake in the population.

TABLE 2
SNP with the high probability of being associated with NFI. The idents are those
in Table 1 and are the ParAllele identifiers. These are located on the BGSP
scaffolds and contigs which indicate the SNP location. These scaffolds and contigs
have been graphically located to maps on the biolives website.

| Ident | BGSP Scaffold | SNP location | Chr. | Contig | Sequence ID | SEQ ID NO: |
|---|---|---|---|---|---|---|
| 345016 | SCAFFOLD291452 | SNP-7280 | Bta2 | Contig126650 | gi51699525 | 1 |
| 349388 | SCAFFOLD261939 | SNP-1026 | Bta1 | Contig290263 | gi21697962 | 2 |
| 346973 | SCAFFOLD71532 | SNP-708 | | contig122162 | gi13397386 | 3 |
| 353336 | SCAFFOLD352562 | SNP-535 | | contig94850 | gi50872235 | 4 |
| 350549 | SCAFFOLD85350 | SNP-16238 | Bta3 | contig324954 | gi13345306 | 5 |
| 347269 | SCAFFOLD165450 | SNP-8470 | Hsa4 | contig222943 | gi19548150 | 6 |
| 350155 | SCAFFOLD240520 | SNP-5556 | Hsa17 | contig32165 | LUC7A | 7 |
| 342650 | IBISS4snp540 | SNP-540 | Hsa1 | | PLA2GA2 | 8 |
| 347570 | SCAFFOLD260891 | SNP-2797 | Hsa9 | contig19628 | | 9 |

TABLE 3
Parallele identifiers for SNP associated with NFI (net feed intake) showing the
sequence scaffold, the Baylor College of Medicine sequence contig containing the SNP, the
Genbank Accession of the DNA sequence, and the alternative bases for each SNP. The exact
location of each SNP is shown in the sequence scaffold, the number after the underscore
is the exact base pair from the start of the scaffold.

| | | | Baylor | | | |
|---|---|---|---|---|---|---|
| Ident | Ibiss4 | Sequence scaffold | Contig | Genbank Accession | SNP bases | SEQ ID NO: |
| 344670 | Null | SCAFFOLD155146_6104 | Contig422955 | AAFC01422942 | A/G | 10 |
| 345385 | Null | SCAFFOLD11677_746 | Contig90789 | AAFC01090787 | C/T | 11 |
| 345533 | Null | SCAFFOLD170036_23917 | Contig433770 | AAFC01433756 | C/T | 12 |
| 348073 | Null | SCAFFOLD110844_5365 | Contig41112 | AAFC01041111 | C/T | 13 |
| 348351 | Null | SCAFFOLD20608_1008 | Contig661138 | AAFC01661110 | C/T | 14 |
| 342844 | Null | SCAFFOLD135269_17123 | Contig131764 | AAFC01131759 | A/G | 15 |
| 342878 | Null | SCAFFOLD155037_16984 | Contig550947 | AAFC01550925 | A/C | 16 |
| 342920 | Null | SCAFFOLD191247_3964 | Contig462712 | AAFC01462697 | C/G | 17 |
| 343301 | Null | SCAFFOLD15017_7532 | Contig168696 | AAFC01168690 | G/T | 18 |
| 343638 | Null | SCAFFOLD466_4857 | Contig366514 | AAFC01366504 | C/G | 19 |
| 343692 | Null | SCAFFOLD71577_11565 | Contig418094 | AAFC01418081 | C/T | 20 |
| 343870 | Null | SCAFFOLD122517_7619 | Contig355194 | AAFC01355184 | C/T | 21 |
| 344238 | Null | SCAFFOLD295006_45304 | Contig19007 | AAFC01019007 | A/G | 22 |
| 344428 | Null | SCAFFOLD86623_4947 | Contig173041 | AAFC01173035 | C/G | 23 |
| 344576 | Null | SCAFFOLD115003_498 | Contig207122 | AAFC01207116 | G/T | 24 |
| 344670 | Null | SCAFFOLD155146_6104 | Contig422955 | AAFC01422942 | A/G | 10 |
| 344822 | Null | SCAFFOLD220929_12275 | Contig726487 | AAFC01726448 | A/G | 25 |
| 345255 | Null | SCAFFOLD91431_3349 | Contig103092 | AAFC01103089 | A/G | 26 |
| 345385 | Null | SCAFFOLD11677_746 | Contig90789 | AAFC01090787 | C/T | 11 |
| 345533 | Null | SCAFFOLD170036_23917 | Contig433770 | AAFC01433756 | C/T | 12 |
| 345597 | Null | SCAFFOLD193911_3423 | Contig10762 | AAFC01010762 | A/G | 27 |
| 346024 | Null | SCAFFOLD49069_4838 | Contig467477 | AAFC01467462 | A/G | 28 |
| 346220 | Null | SCAFFOLD100297_8761 | Contig274152 | AAFC01274143 | G/T | 29 |
| 346800 | Null | SCAFFOLD305898_10192 | Contig158803 | AAFC01158797 | A/G | 30 |
| 346873 | Null | SCAFFOLD38607_6519 | Contig116886 | AAFC01116882 | C/G | 31 |
| 346924 | Null | SCAFFOLD55122_7765 | Contig84561 | AAFC01084560 | A/C | 32 |
| 347029 | Null | SCAFFOLD91431_3586 | Contig103092 | AAFC01103089 | A/C | 33 |
| 347501 | Null | SCAFFOLD245021_7420 | Contig584302 | AAFC01584278 | A/G | 34 |
| 347653 | Null | SCAFFOLD290984_3799 | Contig551036 | AAFC01551014 | C/T | 35 |
| 347727 | Null | SCAFFOLD316858_23449 | Contig113922 | AAFC01113918 | A/G | 36 |
| 348073 | Null | SCAFFOLD110844_5365 | Contig41112 | AAFC01041111 | C/T | 13 |
| 348351 | Null | SCAFFOLD20608_1008 | Contig661138 | AAFC01661110 | C/T | 14 |
| 348465 | Null | SCAFFOLD2481_1870 | Contig499892 | AAFC01499876 | C/T | 37 |
| 348529 | Null | SCAFFOLD268943_3771 | Contig346419 | AAFC01346409 | C/T | 38 |
| 348581 | Null | SCAFFOLD288338_3076 | Contig216746 | AAFC01216740 | C/T | 39 |
| 348678 | Null | SCAFFOLD325650_1207 | Contig637298 | AAFC01637270 | C/T | 40 |
| 348908 | Null | SCAFFOLD10121_13105 | Contig587087 | AAFC01587063 | A/G | 41 |
| 348912 | Null | SCAFFOLD10204_22524 | Contig291510 | AAFC01291500 | A/G | 42 |
| 348917 | Null | SCAFFOLD105096_24896 | Contig256703 | AAFC01256694 | C/T | 43 |
| 349252 | Null | SCAFFOLD216584_13861 | Contig148247 | AAFC01148241 | C/T | 44 |
| 349640 | Null | SCAFFOLD55648_3312 | Contig242188 | AAFC01242181 | C/G | 45 |
| 349908 | Null | SCAFFOLD145774_16171 | Contig83723 | AAFC01083722 | C/T | 46 |
| 350023 | Null | SCAFFOLD190318_15654 | Contig672894 | AAFC01672863 | G/T | 47 |
| 350185 | Null | SCAFFOLD255402_26573 | Contig57357 | AAFC01057356 | C/T | 48 |
| 350386 | Null | SCAFFOLD323674_1755 | Contig684482 | AAFC01684450 | C/T | 49 |
| 350542 | Null | SCAFFOLD85037_17745 | Contig214767 | AAFC01214761 | A/G | 50 |
| 350815 | Null | SCAFFOLD185691_4123 | Contig367991 | AAFC01367981 | A/G | 51 |
| 350934 | Null | SCAFFOLD245150_4016 | Contig79901 | AAFC01079900 | C/T | 52 |
| 351039 | Null | SCAFFOLD285539_13746 | Contig210695 | AAFC01210689 | A/C | 53 |
| 351068 | Null | SCAFFOLD295225_17655 | Contig499260 | AAFC01499244 | C/T | 54 |
| 351082 | Null | SCAFFOLD301148_20414 | Contig399476 | AAFC01399463 | C/T | 55 |
| 351114 | Null | SCAFFOLD317129_13923 | Contig469925 | AAFC01469910 | A/G | 56 |
| 351119 | Null | SCAFFOLD317870_2261 | Contig324071 | AAFC01324061 | C/T | 57 |
| 351157 | Null | SCAFFOLD40038_18553 | Contig442241 | AAFC01442227 | A/T | 58 |
| 351382 | Null | SCAFFOLD115666_13006 | Contig582501 | AAFC01582477 | G/T | 59 |
| 351385 | Null | SCAFFOLD116234_3345 | Contig387450 | AAFC01387438 | C/T | 60 |
| 351524 | Null | SCAFFOLD200011_13411 | Contig569617 | AAFC01569595 | C/T | 61 |
| 351837 | Null | SCAFFOLD48045_564 | Contig135363 | AAFC01135358 | G/T | 62 |
| 351885 | Null | SCAFFOLD6887_2230 | Contig543315 | AAFC01543294 | A/G | 63 |
| 351930 | Null | SCAFFOLD95294_4333 | Contig237582 | AAFC01237575 | A/T | 64 |
| 352079 | Null | SCAFFOLD15666_3538 | Contig545827 | AAFC01545806 | A/G | 65 |
| 352237 | Null | SCAFFOLD250157_11623 | Contig11356 | AAFC01011356 | G/T | 66 |
| 352302 | Null | SCAFFOLD284472_542 | Contig682466 | AAFC01682434 | A/G | 67 |
| 352371 | Null | SCAFFOLD321814_14337 | Contig62446 | AAFC01062445 | C/T | 68 |
| 352540 | Null | SCAFFOLD113475_827 | Contig460868 | AAFC01460853 | A/C | 69 |
| 352721 | Null | SCAFFOLD216584_13710 | Contig148247 | AAFC01148241 | C/T | 70 |
| 352772 | Null | SCAFFOLD25159_19124 | Contig292978 | AAFC01292968 | C/T | 71 |
| 353131 | Null | SCAFFOLD171003_4691 | Contig46420 | AAFC01046419 | A/G | 72 |
| 353697 | Null | SCAFFOLD75727_10176 | Contig497215 | AAFC01497199 | A/G | 73 |
| 353758 | Null | SCAFFOLD130522_9733 | Contig194779 | AAFC01194773 | C/T | 74 |
| 354067 | Null | SCAFFOLD321782_15219 | Contig17844 | AAFC01017844 | C/T | 75 |
| 354212 | Null | SCAFFOLD55122_7983 | Contig84561 | AAFC01084560 | A/C | 76 |
| 354228 | Null | SCAFFOLD75727_10291 | Contig497215 | AAFC01497199 | A/G | 77 |
| 354405 | Null | SCAFFOLD90324_3708 | Contig60222 | AAFC01060221 | C/T | 78 |
| 354433 | Null | SCAFFOLD205065_7657 | Contig650761 | AAFC01650733 | A/T | 79 |

### EXAMPLE 3

To find additional loci affecting net feed intake, further analyses of the whole genome scan were performed. First, the NFI trait values for all measured individuals, not just the 189 in the whole genome scan, were adjusted by contemporary group (herd_cohort_sex_market) and by sireid as a random factor using ASREML. The residual net feed intake phenotypes were then available for further analysis. The means and standard errors of the residual NFI for each genotype in the whole genome scan were calculated and at test computed for each comparison. The statistical significance of the largest mean difference was calculated using 100,000 permutations.

The results of this analysis indicates a further 157 loci of interest (P<0.01) and a further 9 loci with P<0.001 (Table 5).

These loci, as well as some annotation of the 13 top loci, are contained in Table 4. The additional loci show promise as candidate genes for net feed intake. Of these, DNM1L is known to be involved in the size and shape of mitochondria, the powerhouse of the cell, BAAT is known to be involved in differences in the uptake of lipids in the intestine, DMD is known to be involved in muscle growth and hypertrophy, ZNF33A is involved in increased entry into the S or synthesis phase in the cell cycle in which is genome is doubled, and ATP1A1 is known to be involved in the maintenance of the Na+/K+ ion gradient, a process requiring a great deal of the resting metabolic energy. Other speculative candidates are GBAS, which is possibly involved in synaptic vesicle and membrane docking, and maintenance of synaptic vesicle gradients requires a great deal of the resting metabolic energy, and SEC5L1 which is involved in the exocyst complex and also appears to be involved in the neuronal membrane trafficking.

The method of determining whether a measured allele or genotype has an increased value compared to others at that locus or more broadly within the gene or genetic region would be familiar to the person skilled in the art but will be described briefly. In essence we partition the variance associated with the trait into that due to the Mendelian component associated with the locus under discussion as well as a polygenic component due to shared family. Before this is done, the trait values must be adjusted for fixed environmental and genetic effects, for covariates, and for random genetic effects such as the sire or dam. This is usually performed using a General Linear Mixed Model. Then the genotypes can be compared using at test or a one-way analysis of variance, and the statistical significance can be assessed using permutation tests, particularly where the trait distribution is non-normal. The average effect of allele substitution at the locus is derived from the allele frequency, the difference between homozygotes and the degree of dominance, where alpha=a[1+k(p−(1−p)] where a is half the difference between homozygotes, p is the allele frequency and k is d/a where d is the difference between the heterozygote and half the distance between the homozygotes. Good starting points for this process are Boerwinkle et al. 1986 Ann. Hum. Genet. 50, 181-194 and Lynch and Walsh, 1997 (Sinauer Associates), the contents of which are incorporated herein by reference. The association of an allele or genotype with increased value will often apply across breeds and families within breeds. However, a particular allele or genotype may not always be associated with increased value across breeds, in one breed the allele or genotype might be associated with increased value but in another breed it might be associated with decreased value or not be associated with differences in value. The results presented here are the associations aggregated across the breeds, which represents the alleles or genotypes that will be associated with increased value for most breeds. Since the values have been adjusted for the breeds, the associations can be pooled across breeds, and differences in allele frequency in the breeds will not cause the generation of spurious associations due to Simpsons Paradox.

The person skilled in the art will know that some breeds may have different associations between the allele or genotype and the trait due to one of several real biological causes. The first and probably most common is that the measured allele or genotype is not causative, so it is in linkage disequilibrium with the causative allele or genotype. There will be cases where the allele or genotype being measured is in opposite genetic phase to the causative allele or genotype, and this might be reflected in some breed differences. The second is that there may be more than one causative mutation in the gene, with different frequencies in different breeds, hence the measured allele or genotype may show different predictive efficiencies in different breeds and show opposite genetic phase relationships due to complex associations between the measured allele or genotype and the different causative mutations. The third is that a causative mutation in a gene may be affected by genes elsewhere in the genome. These epistatic or background effects have been known for decades, and some of these may have an impact upon the association between the measured allele or genotype and the trait value.

TABLE 4
Loci with high levels of support associated with adjusted net feed intake.

| | | | | | | SEQ |
|---|---|---|---|---|---|---|
| | scaffold- | | | | | ID |
| snp_id | v2 | bp | PermP | Hsa | Gene | NO: |
| 354186 | Un.449 | 364840 | 0.0001 | X | DMD, Duchenne Muscular Dystrophy | 80 |
| 350075 | Un.7108 | 39426 | 0.0002 | 5 | no gene near | 81 |
| 345371 | Un.2454 | 65434 | 0.0003 | 3 | EMR2, EGF-like, mucin-like, hormone receptor like 2 | 82 |
| 349676 | Un.2314 | 29810 | 0.0004 | 3 | no gene near | 83 |
| 350777 | 28.18 | 1679163 | 0.0006 | 1 | ZNF33A, zinc finger protein 33A | 84 |
| 344359 | 5.117 | 1267082 | 0.0007 | 12 | DNM1L, dynamin 1-like | 85 |
| 345126 | 15.107 | 9296 | 0.0008 | 11 | near C11orf8 | 86 |
| 347062 | 25.53 | 689761 | 0.0008 | 7 | GBAS glioblastoma amplified sequence | 87 |
| 348806 | Un.204 | 76583 | 0.0011 | 6 | SEC5L1 SEC5-like 1 (S. cerevisiae) | 88 |
| 343136 | 3.6 | 42829 | 0.0012 | 1 | ATP1A1, ATPase, Na+/K+ transporting, alpha 1 | 89 |
| | | | | | polypeptide | |
| 348798 | Un.2314 | 29908 | 0.0012 | 3 | no gene near | 90 |
| 349583 | 4.96 | 282594 | 0.0012 | 7 | no gene near | 91 |
| 344029 | 14.72 | 324549 | 0.0031 | 8 | BAAT bile acid coenzyme A amino acid N- | 92 |
| | | | | | acyltransferase | |

Snp_id is the ParAllele SNP identifier,
scaffold-v2 is the version 2 scaffold,
bp is the location of the SNP in the scaffold,
PermP is the P value associated with the association,
Hsa is the human chromosome associated with the SNP and Gene is the closest gene to the SNP.

TABLE 5
Associations between DNA markers and net feed intake sorted in decreasing order of
statistical significance.

| Locus | mean_0 | SE | mean_1 | SE | mean_2 | SE | N | Freq | a | k | alpha | tmax | PermP |
|---|---|---|---|---|---|---|---|---|---|---|---|---|---|
| 353336 | 1.37 | 0.33 | 0.03 | 0.20 | −0.04 | 0.12 | 171 | 0.20 | 0.70 | −0.91 | 1.08 | 3.87 | 0.0000 |
| 354433 | 0.17 | 0.32 | −1.41 | 0.32 | 0.19 | 0.11 | 161 | 0.07 | −0.01 | 146.52 | 1.37 | 3.63 | 0.0000 |
| 354186 | −0.13 | 0.11 | NaN | NaN | 0.67 | 0.20 | 165 | 0.75 | −0.40 | NaN | NaN | 3.50 | 0.0001 |
| 343692 | 0.00 | 0.15 | 0.48 | 0.17 | −0.51 | 0.17 | 170 | 0.62 | 0.26 | 2.83 | 0.43 | 3.60 | 0.0001 |
| 347269 | −1.33 | 0.34 | 0.28 | 0.17 | 0.11 | 0.12 | 171 | 0.19 | −0.72 | −1.23 | −1.27 | 3.64 | 0.0002 |
| 350075 | −0.26 | 0.16 | 0.47 | 0.15 | 0.08 | 0.21 | 167 | 0.54 | −0.17 | −3.33 | −0.13 | 3.31 | 0.0002 |
| 347570 | 0.76 | 0.24 | 0.09 | 0.15 | −0.24 | 0.15 | 170 | 0.40 | 0.50 | −0.34 | 0.54 | 3.68 | 0.0002 |
| 343617 | −0.05 | 0.14 | −0.06 | 0.19 | 1.09 | 0.28 | 150 | 0.72 | −0.57 | 1.02 | −0.83 | 3.42 | 0.0003 |
| 345371 | 1.28 | 0.33 | −0.20 | 0.23 | 0.06 | 0.11 | 171 | 0.14 | 0.61 | −1.42 | 1.25 | 3.52 | 0.0003 |
| 349676 | 0.63 | 0.22 | −0.25 | 0.14 | 0.21 | 0.17 | 170 | 0.44 | 0.21 | −3.14 | 0.30 | 3.41 | 0.0004 |
| 350777 | −0.03 | 0.11 | 0.53 | 0.22 | 1.87 | 0.60 | 168 | 0.90 | −0.95 | 0.41 | −1.26 | 3.21 | 0.0006 |
| 347727 | −0.23 | 0.13 | 0.51 | 0.18 | 0.11 | 0.27 | 168 | 0.66 | −0.17 | −3.41 | 0.02 | 3.48 | 0.0007 |
| 344359 | −0.38 | 0.19 | 0.16 | 0.18 | 0.34 | 0.14 | 171 | 0.44 | −0.36 | −0.51 | −0.38 | 3.04 | 0.0007 |
| 345126 | 1.35 | 0.61 | −0.83 | 0.35 | 0.17 | 0.10 | 169 | 0.06 | 0.59 | −2.71 | 1.98 | 3.05 | 0.0008 |
| 347062 | 0.67 | 0.24 | 0.08 | 0.14 | −0.26 | 0.16 | 169 | 0.44 | 0.46 | −0.26 | 0.48 | 3.33 | 0.0008 |
| 351930 | 0.83 | 0.31 | −0.14 | 0.16 | 0.03 | 0.14 | 163 | 0.30 | 0.40 | −1.43 | 0.62 | 2.96 | 0.0009 |
| 350155 | 0.37 | 0.16 | −0.30 | 0.14 | 0.56 | 0.25 | 170 | 0.61 | −0.09 | 8.16 | −0.26 | 3.20 | 0.0010 |
| 348806 | 0.08 | 0.10 | 2.37 | 0.74 | NaN | NaN | 161 | 0.99 | NaN | NaN | NaN | 3.10 | 0.0011 |
| 350549 | 0.76 | 0.24 | 0.15 | 0.15 | −0.35 | 0.22 | 137 | 0.44 | 0.55 | −0.10 | 0.56 | 3.23 | 0.0011 |
| 348798 | 0.21 | 0.17 | −0.28 | 0.15 | 0.62 | 0.23 | 168 | 0.57 | −0.21 | 3.33 | −0.30 | 3.43 | 0.0012 |
| 349583 | 1.61 | 0.54 | −0.31 | 0.21 | 0.10 | 0.11 | 169 | 0.09 | 0.76 | −1.53 | 1.72 | 3.88 | 0.0012 |
| 343136 | −0.22 | 0.14 | 0.21 | 0.16 | 0.75 | 0.24 | 170 | 0.66 | −0.48 | 0.11 | −0.50 | 3.50 | 0.0012 |
| 343870 | −0.41 | 0.18 | 0.35 | 0.19 | 0.24 | 0.16 | 160 | 0.45 | −0.33 | −1.33 | −0.37 | 2.93 | 0.0013 |
| 344786 | 0.03 | 0.16 | 0.72 | 0.24 | −0.18 | 0.16 | 157 | 0.54 | 0.11 | 7.41 | 0.16 | 3.17 | 0.0013 |
| 347283 | −0.76 | 0.23 | 0.29 | 0.20 | 0.15 | 0.13 | 167 | 0.25 | −0.46 | −1.30 | −0.76 | 3.04 | 0.0014 |
| 353387 | 0.21 | 0.11 | −0.67 | 0.29 | −0.36 | 0.50 | 166 | 0.89 | 0.28 | −2.08 | −0.17 | 2.87 | 0.0014 |
| 351702 | 0.69 | 0.21 | NaN | NaN | −0.11 | 0.11 | 170 | 0.24 | 0.40 | NaN | NaN | 3.53 | 0.0014 |
| 346024 | 0.07 | 0.13 | 0.53 | 0.18 | −0.44 | 0.25 | 171 | 0.72 | 0.26 | 2.76 | 0.57 | 3.22 | 0.0015 |
| 345292 | 0.20 | 0.20 | −0.21 | 0.13 | 0.50 | 0.20 | 168 | 0.53 | −0.15 | 3.65 | −0.19 | 3.06 | 0.0016 |
| 351344 | 1.11 | 0.37 | −0.26 | 0.17 | 0.18 | 0.13 | 170 | 0.23 | 0.46 | −1.95 | 0.95 | 3.26 | 0.0017 |
| 342844 | 0.09 | 0.11 | 0.73 | 0.25 | −0.56 | 0.28 | 165 | 0.88 | 0.33 | 2.96 | 1.07 | 3.49 | 0.0018 |
| 346973 | −0.36 | 0.22 | 0.64 | 0.20 | 0.04 | 0.14 | 163 | 0.30 | −0.20 | −3.97 | −0.52 | 3.32 | 0.0018 |
| 350444 | 0.12 | 0.14 | −0.51 | 0.24 | 0.36 | 0.18 | 166 | 0.66 | −0.12 | 6.37 | −0.35 | 2.93 | 0.0018 |
| 346987 | −0.11 | 0.55 | 0.62 | 0.15 | −0.11 | 0.13 | 167 | 0.16 | −0.00 | −1228.83 | −0.50 | 3.24 | 0.0019 |
| 346634 | −0.17 | 0.14 | 0.13 | 0.16 | 0.67 | 0.24 | 168 | 0.62 | −0.42 | 0.30 | −0.45 | 3.06 | 0.0019 |
| 348902 | 1.42 | 0.53 | −0.29 | 0.22 | 0.12 | 0.11 | 171 | 0.18 | 0.65 | −1.63 | 1.34 | 3.16 | 0.0019 |
| 349297 | −0.25 | 0.18 | 0.15 | 0.15 | 0.62 | 0.18 | 168 | 0.60 | −0.43 | 0.08 | −0.44 | 3.16 | 0.0019 |
| 347122 | −0.29 | 0.14 | 0.29 | 0.16 | 0.49 | 0.23 | 168 | 0.60 | −0.39 | −0.50 | −0.35 | 3.05 | 0.0020 |
| 349908 | 0.09 | 0.24 | 0.35 | 0.15 | −0.28 | 0.15 | 170 | 0.46 | 0.18 | 2.43 | 0.14 | 2.95 | 0.0021 |
| 349965 | −0.74 | 0.25 | 0.31 | 0.18 | 0.13 | 0.14 | 162 | 0.28 | −0.44 | −1.41 | −0.71 | 3.30 | 0.0022 |
| 345591 | 1.18 | 0.55 | 0.09 | 0.22 | −0.00 | 0.11 | 170 | 0.19 | 0.59 | −0.85 | 0.90 | 2.95 | 0.0025 |
| 350995 | 0.12 | 0.11 | −1.61 | 0.19 | 0.04 | 0.58 | 143 | 0.96 | 0.04 | −42.41 | −1.52 | 3.33 | 0.0026 |
| 347733 | −0.12 | 0.14 | 0.06 | 0.16 | 0.73 | 0.29 | 170 | 0.66 | −0.42 | 0.59 | −0.50 | 2.94 | 0.0026 |
| 345261 | 0.04 | 0.11 | 0.46 | 0.23 | −1.79 | 0.55 | 170 | 0.89 | 0.91 | 1.47 | 1.96 | 2.93 | 0.0027 |
| 348912 | 0.01 | 0.11 | 0.80 | 0.24 | −0.65 | 0.48 | 169 | 0.86 | 0.33 | 3.37 | 1.12 | 2.98 | 0.0027 |
| 346106 | −0.07 | 0.12 | 0.23 | 0.20 | 1.02 | 0.41 | 170 | 0.80 | −0.55 | 0.44 | −0.69 | 2.69 | 0.0030 |
| 343500 | 0.07 | 0.10 | −0.11 | 0.31 | 1.45 | 0.56 | 169 | 0.88 | −0.69 | 1.26 | −1.36 | 2.92 | 0.0030 |
| 343193 | −0.42 | 0.23 | 0.03 | 0.16 | 0.41 | 0.15 | 169 | 0.40 | −0.41 | −0.09 | −0.42 | 3.04 | 0.0030 |
| 344029 | −0.54 | 0.28 | 0.39 | 0.18 | 0.06 | 0.13 | 170 | 0.30 | −0.30 | −2.14 | −0.56 | 2.83 | 0.0031 |
| 353932 | 0.83 | 0.33 | 0.33 | 0.17 | −0.17 | 0.13 | 171 | 0.26 | 0.50 | 0.00 | 0.50 | 2.95 | 0.0032 |
| 346573 | −0.10 | 0.20 | 0.45 | 0.18 | −0.17 | 0.14 | 170 | 0.35 | 0.03 | 17.90 | −0.14 | 2.79 | 0.0032 |
| 353748 | 0.11 | 0.14 | −0.26 | 0.18 | 0.51 | 0.20 | 171 | 0.62 | −0.20 | 2.83 | −0.34 | 2.80 | 0.0032 |
| 345696 | −0.19 | 0.15 | 0.45 | 0.19 | 0.17 | 0.20 | 160 | 0.63 | −0.18 | −2.62 | −0.06 | 2.71 | 0.0033 |
| 352784 | −0.86 | 0.35 | 0.31 | 0.19 | 0.12 | 0.12 | 169 | 0.18 | −0.49 | −1.38 | −0.92 | 3.05 | 0.0035 |
| 345464 | 0.03 | 0.10 | 1.09 | 0.38 | −0.88 | 0.15 | 170 | 0.95 | 0.45 | 3.35 | 1.81 | 2.75 | 0.0035 |
| 344027 | 0.84 | 0.49 | −0.61 | 0.21 | 0.20 | 0.11 | 170 | 0.14 | 0.32 | −3.53 | 1.14 | 3.15 | 0.0035 |
| 346642 | 0.55 | 0.23 | −0.21 | 0.14 | 0.13 | 0.16 | 168 | 0.43 | 0.21 | −2.65 | 0.29 | 2.97 | 0.0037 |
| 343918 | 0.26 | 0.24 | 0.35 | 0.14 | −0.30 | 0.16 | 171 | 0.44 | 0.28 | 1.32 | 0.23 | 3.04 | 0.0039 |
| 346964 | 0.10 | 0.15 | −0.14 | 0.14 | 0.73 | 0.28 | 171 | 0.62 | −0.31 | 1.76 | −0.45 | 2.95 | 0.0039 |
| 352451 | −0.49 | 0.20 | 0.24 | 0.15 | 0.21 | 0.16 | 170 | 0.44 | −0.35 | −1.08 | −0.39 | 2.73 | 0.0040 |
| 347425 | −0.28 | 0.19 | 0.38 | 0.16 | 0.16 | 0.17 | 170 | 0.50 | −0.22 | −2.02 | −0.22 | 2.72 | 0.0040 |
| 342614 | 0.01 | 0.10 | 1.00 | 0.31 | −0.02 | 0.96 | 171 | 0.95 | 0.01 | 68.20 | 0.92 | 2.77 | 0.0040 |
| 344675 | 0.12 | 0.14 | 0.25 | 0.17 | −0.88 | 0.32 | 166 | 0.76 | 0.50 | 1.26 | 0.83 | 3.03 | 0.0041 |
| 345016 | −0.16 | 0.17 | −0.00 | 0.15 | 0.64 | 0.20 | 170 | 0.57 | −0.40 | 0.61 | −0.43 | 3.04 | 0.0042 |
| 346191 | 0.29 | 0.32 | −0.16 | 0.14 | 0.48 | 0.18 | 151 | 0.37 | −0.10 | 5.45 | 0.04 | 2.89 | 0.0042 |
| 344550 | 0.10 | 0.10 | 1.61 | 0.55 | −0.21 | 2.48 | 158 | 0.97 | 0.15 | 10.97 | 1.72 | 2.66 | 0.0042 |
| 344923 | 0.52 | 0.22 | −0.22 | 0.15 | 0.13 | 0.16 | 170 | 0.43 | 0.19 | −2.81 | 0.27 | 2.88 | 0.0043 |
| 353129 | 0.15 | 0.13 | 0.24 | 0.19 | −0.81 | 0.24 | 169 | 0.75 | 0.48 | 1.18 | 0.77 | 3.00 | 0.0043 |
| 350612 | −0.61 | 0.28 | 0.28 | 0.16 | 0.10 | 0.14 | 171 | 0.31 | −0.36 | −1.49 | −0.56 | 2.76 | 0.0044 |
| 348684 | −0.84 | 0.37 | 0.10 | 0.17 | 0.21 | 0.13 | 169 | 0.25 | −0.53 | −0.78 | −0.74 | 2.75 | 0.0045 |
| 349112 | 0.13 | 0.10 | 0.43 | 0.52 | −1.75 | 0.52 | 159 | 0.95 | 0.94 | 1.32 | 2.05 | 2.95 | 0.0045 |
| 342958 | −0.41 | 0.19 | 0.27 | 0.15 | 0.28 | 0.21 | 161 | 0.52 | −0.34 | −0.99 | −0.33 | 2.81 | 0.0045 |
| 344238 | −0.04 | 0.13 | 0.06 | 0.17 | 0.84 | 0.30 | 170 | 0.71 | −0.44 | 0.77 | −0.58 | 2.79 | 0.0046 |
| 346975 | −0.04 | 0.71 | 0.68 | 0.23 | −0.05 | 0.11 | 171 | 0.13 | 0.01 | 107.69 | −0.53 | 2.91 | 0.0046 |
| 347075 | 0.53 | 0.19 | −0.17 | 0.14 | 0.16 | 0.20 | 171 | 0.46 | 0.18 | −2.77 | 0.22 | 2.97 | 0.0048 |
| 345781 | −0.01 | 0.13 | 0.04 | 0.17 | 0.95 | 0.37 | 171 | 0.72 | −0.48 | 0.88 | −0.67 | 2.71 | 0.0048 |
| 350926 | −0.35 | 0.20 | 0.37 | 0.14 | −0.02 | 0.18 | 168 | 0.46 | −0.16 | −3.43 | −0.21 | 2.88 | 0.0049 |
| 349265 | −0.26 | 0.17 | 0.34 | 0.13 | −0.05 | 0.26 | 170 | 0.56 | −0.11 | −4.71 | −0.05 | 2.81 | 0.0049 |
| 351382 | 0.08 | 0.12 | 0.40 | 0.21 | −0.98 | 0.31 | 162 | 0.80 | 0.53 | 1.60 | 1.05 | 2.84 | 0.0050 |
| 348351 | −0.89 | 0.24 | 0.37 | 0.21 | 0.08 | 0.12 | 171 | 0.21 | −0.49 | −1.59 | −0.93 | 3.08 | 0.0050 |
| 343949 | 0.11 | 0.11 | 0.24 | 0.28 | −1.92 | 0.38 | 170 | 0.93 | 1.02 | 1.12 | 2.00 | 3.02 | 0.0050 |
| 353131 | 0.40 | 0.17 | 0.04 | 0.16 | −0.31 | 0.18 | 169 | 0.57 | 0.36 | −0.01 | 0.36 | 2.77 | 0.0052 |
| 345458 | 0.06 | 0.28 | −0.24 | 0.14 | 0.36 | 0.16 | 161 | 0.32 | −0.15 | 3.01 | 0.01 | 2.71 | 0.0054 |
| 345255 | 0.38 | 0.20 | 0.06 | 0.13 | −0.39 | 0.22 | 166 | 0.54 | 0.39 | 0.15 | 0.39 | 2.51 | 0.0056 |
| 345916 | 0.27 | 0.12 | −0.44 | 0.20 | −0.12 | 0.38 | 168 | 0.83 | 0.20 | −2.66 | −0.15 | 2.82 | 0.0056 |
| 346166 | 0.16 | 0.20 | −0.19 | 0.14 | 0.49 | 0.20 | 167 | 0.54 | −0.17 | 3.07 | −0.21 | 2.80 | 0.0056 |
| 346330 | −0.34 | 0.16 | 0.30 | 0.19 | 0.36 | 0.17 | 162 | 0.52 | −0.35 | −0.81 | −0.34 | 3.00 | 0.0057 |
| 353072 | 0.07 | 0.11 | 0.64 | 0.27 | −1.12 | 0.57 | 167 | 0.92 | 0.59 | 1.96 | 1.56 | 3.01 | 0.0057 |
| 350500 | 0.08 | 0.12 | −0.06 | 0.18 | 1.26 | 0.41 | 170 | 0.77 | −0.59 | 1.24 | −0.98 | 2.78 | 0.0058 |
| 344038 | −0.98 | 0.41 | 0.36 | 0.40 | 0.12 | 0.11 | 165 | 0.11 | −0.55 | −1.43 | −1.16 | 2.77 | 0.0059 |
| 351664 | −0.06 | 0.11 | 0.64 | 0.22 | −0.22 | 0.36 | 171 | 0.85 | 0.08 | 9.86 | 0.64 | 2.97 | 0.0060 |
| 349617 | 0.28 | 0.12 | −0.38 | 0.19 | 0.16 | 0.32 | 170 | 0.78 | 0.06 | −10.76 | −0.28 | 2.90 | 0.0060 |
| 353087 | 0.19 | 0.91 | 0.86 | 0.26 | −0.02 | 0.11 | 169 | 0.07 | 0.10 | 7.42 | −0.57 | 2.71 | 0.0061 |
| 351855 | 0.38 | 0.15 | 0.09 | 0.19 | −0.23 | 0.17 | 170 | 0.50 | 0.30 | 0.04 | 0.30 | 2.60 | 0.0061 |
| 346539 | 0.87 | 0.24 | 0.18 | 0.19 | −0.08 | 0.13 | 169 | 0.25 | 0.47 | −0.47 | 0.58 | 2.83 | 0.0061 |
| 350312 | 0.10 | 0.19 | −0.17 | 0.15 | 0.51 | 0.18 | 170 | 0.55 | −0.21 | 2.28 | −0.25 | 2.85 | 0.0064 |
| 352162 | −0.22 | 0.15 | 0.43 | 0.17 | −0.07 | 0.19 | 170 | 0.56 | −0.07 | −7.91 | −0.00 | 2.83 | 0.0066 |
| 345166 | 0.25 | 0.12 | 0.02 | 0.20 | −0.58 | 0.29 | 171 | 0.73 | 0.42 | 0.44 | 0.50 | 2.78 | 0.0067 |
| 347646 | 0.02 | 0.12 | −0.01 | 0.22 | 1.08 | 0.37 | 167 | 0.80 | −0.53 | 1.06 | −0.87 | 2.78 | 0.0067 |
| 354124 | NaN | NaN | 1.69 | 0.40 | 0.03 | 0.10 | 170 | 0.01 | NaN | NaN | NaN | 2.84 | 0.0067 |
| 348221 | −0.33 | 0.15 | 0.33 | 0.15 | 0.12 | 0.20 | 171 | 0.47 | −0.23 | −1.90 | −0.25 | 2.89 | 0.0069 |
| 345428 | −0.14 | 0.14 | 0.16 | 0.15 | 0.73 | 0.31 | 166 | 0.67 | −0.44 | 0.33 | −0.49 | 2.73 | 0.0069 |
| 346083 | −0.10 | 0.12 | 0.45 | 0.17 | 0.10 | 0.48 | 170 | 0.76 | −0.10 | −4.74 | 0.14 | 2.59 | 0.0069 |
| 350535 | −0.04 | 0.34 | 0.70 | 0.22 | −0.02 | 0.12 | 170 | 0.14 | −0.01 | −60.99 | −0.54 | 2.48 | 0.0070 |
| 346860 | 0.27 | 0.12 | −0.31 | 0.19 | −0.19 | 0.33 | 171 | 0.80 | 0.23 | −1.53 | 0.02 | 2.54 | 0.0070 |
| 348791 | −0.08 | 0.17 | −0.10 | 0.14 | 0.59 | 0.22 | 170 | 0.52 | −0.34 | 1.03 | −0.35 | 2.77 | 0.0072 |
| 353906 | 0.04 | 0.30 | 0.42 | 0.15 | −0.20 | 0.14 | 170 | 0.35 | 0.12 | 4.16 | −0.03 | 2.98 | 0.0074 |
| 352079 | 0.14 | 0.16 | 0.47 | 0.23 | −0.23 | 0.16 | 160 | 0.56 | 0.18 | 2.80 | 0.24 | 2.57 | 0.0074 |
| 349343 | 1.97 | 0.65 | 0.20 | 0.20 | 0.03 | 0.12 | 168 | 0.14 | 0.97 | −0.83 | 1.56 | 2.56 | 0.0075 |
| 348449 | −0.04 | 0.18 | 0.39 | 0.15 | −0.35 | 0.20 | 168 | 0.55 | 0.15 | 3.80 | 0.21 | 2.91 | 0.0075 |
| 346924 | 0.29 | 0.14 | 0.03 | 0.17 | −0.54 | 0.25 | 168 | 0.68 | 0.41 | 0.37 | 0.47 | 2.63 | 0.0076 |
| 349798 | 0.03 | 0.14 | 0.36 | 0.16 | −0.54 | 0.27 | 170 | 0.76 | 0.28 | 2.15 | 0.61 | 2.68 | 0.0076 |
| 346663 | 0.09 | 0.11 | 0.46 | 0.25 | −1.13 | 0.22 | 170 | 0.93 | 0.61 | 1.62 | 1.45 | 3.05 | 0.0076 |
| 352113 | 0.09 | 0.11 | 0.40 | 0.24 | −1.13 | 0.22 | 171 | 0.93 | 0.61 | 1.52 | 1.39 | 2.93 | 0.0077 |
| 351627 | −0.91 | 0.34 | 0.39 | 0.21 | 0.05 | 0.12 | 168 | 0.17 | −0.48 | −1.72 | −1.02 | 2.78 | 0.0077 |
| 353576 | 1.03 | 0.45 | −0.05 | 0.17 | 0.07 | 0.12 | 165 | 0.19 | 0.48 | −1.26 | 0.86 | 2.62 | 0.0078 |
| 348728 | 0.15 | 0.18 | −0.26 | 0.17 | 0.40 | 0.18 | 158 | 0.54 | −0.13 | 4.20 | −0.17 | 2.60 | 0.0079 |
| 348913 | 0.27 | 0.14 | 0.23 | 0.18 | −0.38 | 0.22 | 169 | 0.59 | 0.33 | 0.87 | 0.38 | 2.65 | 0.0079 |
| 349578 | −0.39 | 0.31 | 0.57 | 0.19 | −0.03 | 0.12 | 169 | 0.25 | −0.18 | −4.39 | −0.57 | 2.70 | 0.0080 |
| 347472 | 0.42 | 0.18 | −0.12 | 0.15 | 0.08 | 0.20 | 170 | 0.51 | 0.17 | −2.21 | 0.17 | 2.31 | 0.0082 |
| 349376 | −1.13 | 0.22 | 0.40 | 0.24 | 0.09 | 0.11 | 171 | 0.07 | −0.61 | −1.52 | −1.39 | 2.93 | 0.0082 |
| 350721 | 0.29 | 0.13 | −0.26 | 0.17 | 0.18 | 0.33 | 171 | 0.71 | 0.06 | −8.62 | −0.15 | 2.60 | 0.0082 |
| 347029 | 0.41 | 0.19 | 0.04 | 0.14 | −0.36 | 0.22 | 164 | 0.55 | 0.38 | 0.03 | 0.38 | 2.60 | 0.0083 |
| 346682 | 0.04 | 0.13 | 0.29 | 0.16 | −1.13 | 0.25 | 171 | 0.80 | 0.58 | 1.44 | 1.10 | 2.53 | 0.0084 |
| 348436 | 0.02 | 0.11 | 0.57 | 0.26 | −1.58 | 0.56 | 170 | 0.89 | 0.80 | 1.69 | 1.86 | 2.52 | 0.0084 |
| 354212 | 0.29 | 0.14 | 0.04 | 0.16 | −0.54 | 0.25 | 171 | 0.68 | 0.41 | 0.40 | 0.47 | 2.63 | 0.0084 |
| 349604 | 0.32 | 0.15 | 0.14 | 0.18 | −0.33 | 0.17 | 169 | 0.58 | 0.33 | 0.46 | 0.35 | 2.73 | 0.0085 |
| 344696 | 0.23 | 0.46 | −0.31 | 0.17 | 0.26 | 0.13 | 170 | 0.20 | −0.02 | 33.79 | 0.31 | 2.60 | 0.0085 |
| 347934 | 0.28 | 0.13 | 0.03 | 0.17 | −0.47 | 0.30 | 168 | 0.68 | 0.37 | 0.33 | 0.42 | 2.46 | 0.0086 |
| 354173 | 0.03 | 0.15 | −0.24 | 0.21 | 0.41 | 0.17 | 167 | 0.48 | −0.19 | 2.39 | −0.17 | 2.41 | 0.0086 |
| 353490 | 0.58 | 0.22 | −0.06 | 0.16 | −0.01 | 0.17 | 155 | 0.45 | 0.30 | −1.15 | 0.33 | 2.31 | 0.0086 |
| 351064 | 1.07 | 0.39 | 1.17 | 0.54 | −0.03 | 0.11 | 139 | 0.06 | 0.55 | 1.19 | −0.02 | 2.56 | 0.0087 |
| 345918 | −0.12 | 0.24 | −0.18 | 0.16 | 0.38 | 0.14 | 163 | 0.35 | −0.25 | 1.23 | −0.16 | 2.57 | 0.0088 |
| 346960 | 0.53 | 0.19 | 0.04 | 0.17 | −0.12 | 0.16 | 169 | 0.42 | 0.32 | −0.51 | 0.35 | 2.52 | 0.0088 |
| 345383 | 0.13 | 0.12 | 0.26 | 0.20 | −0.82 | 0.24 | 169 | 0.81 | 0.48 | 1.27 | 0.85 | 2.94 | 0.0088 |
| 342761 | 0.41 | 0.21 | −0.28 | 0.17 | 0.20 | 0.15 | 170 | 0.31 | 0.10 | −5.81 | 0.32 | 2.46 | 0.0088 |
| 346264 | −0.27 | 0.19 | 0.05 | 0.16 | 0.34 | 0.17 | 169 | 0.46 | −0.31 | −0.03 | −0.31 | 2.42 | 0.0090 |
| 345385 | 0.01 | 0.16 | 0.47 | 0.19 | −0.24 | 0.14 | 169 | 0.61 | 0.12 | 4.82 | 0.25 | 2.74 | 0.0090 |
| 342912 | −0.46 | 0.96 | 0.98 | 0.54 | −0.04 | 0.11 | 145 | 0.07 | −0.21 | −5.80 | −1.26 | 2.50 | 0.0091 |
| 352161 | 0.17 | 0.10 | −0.79 | 0.33 | −0.74 | NaN | 171 | 0.95 | 0.45 | −1.11 | −0.00 | 2.66 | 0.0091 |
| 345811 | 0.34 | 0.13 | −0.23 | 0.21 | −0.03 | 0.31 | 145 | 0.71 | 0.19 | −2.05 | 0.03 | 2.48 | 0.0091 |
| 353614 | −0.11 | 0.12 | NaN | NaN | 0.41 | 0.16 | 171 | 0.62 | −0.26 | NaN | NaN | 2.54 | 0.0092 |
| 348529 | −0.25 | 0.20 | 0.05 | 0.15 | 0.54 | 0.22 | 158 | 0.48 | −0.40 | 0.25 | −0.39 | 2.63 | 0.0092 |
| 352870 | 0.55 | 0.57 | −0.22 | 0.15 | 0.28 | 0.13 | 171 | 0.28 | 0.13 | −4.78 | 0.41 | 2.56 | 0.0092 |
| 345597 | 0.34 | 2.08 | −0.56 | 0.23 | 0.17 | 0.11 | 170 | 0.08 | 0.09 | −9.49 | 0.77 | 2.55 | 0.0093 |
| 348888 | 0.12 | 0.18 | 0.19 | 0.13 | −0.73 | 0.32 | 170 | 0.66 | 0.42 | 1.17 | 0.58 | 2.48 | 0.0094 |
| 348400 | 0.95 | 0.41 | 0.35 | 0.19 | −0.09 | 0.12 | 170 | 0.20 | 0.52 | −0.15 | 0.57 | 2.69 | 0.0094 |
| 348820 | 0.15 | 0.14 | −0.32 | 0.20 | 0.37 | 0.21 | 170 | 0.66 | −0.11 | 5.30 | −0.30 | 2.37 | 0.0095 |
| 346608 | −0.13 | 0.13 | 0.42 | 0.18 | −0.03 | 0.23 | 169 | 0.62 | −0.05 | −10.57 | 0.07 | 2.47 | 0.0095 |
| 348360 | 0.49 | 0.31 | −0.70 | 0.33 | 0.17 | 0.10 | 171 | 0.06 | 0.16 | −6.27 | 1.07 | 2.55 | 0.0095 |
| 350976 | −0.11 | 0.13 | 0.45 | 0.20 | 0.16 | 0.25 | 171 | 0.69 | −0.13 | −3.14 | 0.02 | 2.41 | 0.0097 |
| 349603 | 0.05 | 0.12 | −0.10 | 0.20 | 0.85 | 0.30 | 170 | 0.78 | −0.40 | 1.38 | −0.71 | 2.56 | 0.0097 |
| 347423 | 0.25 | 0.13 | −0.30 | 0.16 | 0.60 | 0.43 | 170 | 0.76 | −0.18 | 4.10 | −0.55 | 2.62 | 0.0097 |
| 349988 | 0.29 | 0.14 | −0.26 | 0.16 | 0.34 | 0.26 | 171 | 0.68 | −0.03 | 20.29 | −0.24 | 2.53 | 0.0099 |
| 343688 | 0.67 | 0.32 | −0.54 | 0.24 | 0.14 | 0.12 | 167 | 0.13 | 0.27 | −3.54 | 0.95 | 2.80 | 0.0099 |
| 352122 | 0.29 | 0.14 | −0.26 | 0.16 | 0.34 | 0.26 | 171 | 0.68 | −0.03 | 20.29 | −0.24 | 2.53 | 0.0100 |

Locus is the ParAllele identifiers of the polymorphisms,
N is the number of genotypes,
mean_0 is the mean net feed intake for genotype 0,
mean_1 is the mean for genotype 1, and
mean_2 is the mean of genotype 2,
SE is the standard error,
a is the additive effect,
k is the dominance effect,
alpha is the average effect of allele substitutions,
tmax is the value of the t test, and
PermP is the P value determined from 100,000 permutation tests expressed as a positive integer.
A minus value for a means the mean of genotype 0 is lower than the mean of genotype 2, and a minus value for alpha means that selecting for allele 0 will reduce the average values of net feed intake in the population.

TABLE 6
DNA Sequences for SNPs from Table 4

| | V1 | | | | |
|---|---|---|---|---|---|
| | Scaffold | | Alter- | | |
| | or | | na- | | SEQ |
| | IBISS4 | | tive | | ID |
| Locus | No. | 5′ Flanking Sequence | Bases | 3′ Flanking Sequence | NO: |
| 354186 | SCAFFOLD | TTCCTGTAAGAAGAATGAGAGTTATAATATGCAAATATA | G/A | ATCAATATATTCTATAGATTGCAATTCATTTGTAATGAA | 80 |
| | 285370_ | TCATATTAATGGTATATTATATTTATTTGATGACATCTT | | AAACTATAAATGTATTGCAGGCTGTGCCCCAAACATACT | |
| | 4357 | TTCCAATTAAATAGGAAGAATTACATTTGAACAAATCTC | | TGCTCTATTGTCTGTACCTTCTTTTGTTCTTCCTTCTCC | |
| | | ATAAATTAAACAATGCCTTCCTCTGTTTTGTTGAAATTT | | CTGGATGTGTAGGTAAGGGATGTCACCTTACAGAGAAAT | |
| | | TTAATAATTTTATTTGATTAGTGGGTTACTATTTAAAGA | | TCCTTCAGGGTCTTTGGAAAAATAGGAACTGAGTTATTT | |
| | | ATCCTCAAACACTATAGCTTCTGTCTATACAATTGATTT | | CACTCATAGCCTCTCCAACCCCCTCTGTCAGGCTCAGCT | |
| | | GTTCATTTTTTTATGA | | TTCCTCAGTCACTCTG | |
| 350075 | SCAFFOLD | TGTATTTGAAAACTTGTTAAGGTTTATTACATTTCTAAT | A/C | AGGCAATGGCACCCCACTCCTGTACTCTTGCCTGGAAAA | 81 |
| | 205874_ | ATAAGAAGAGATAATAGGTAAAGGATAAGATACAAACTT | | TCCATGGACAGAGGAGCCAGGTAGGCTGCAGTTCATGGG | |
| | 730 | GAACAAAGGTCAGTAGGTTCAGTCTGTATACAGAGGAGC | | GTCGCTAAGAGTCCGACACGACTAAGGGACTTCACTTTC | |
| | | AGAAAGTTATCCCTGTGTTTCTGATTTAACTTAGACAAA | | ACTTTTCACTTTCATGCATTGGAGAAGGAAATGGCAGCC | |
| | | AAATGGGAGCAAAGAAAGGGGAAGATCTAGAATTTTTAA | | CACTCCAGTGTTCTTGCCTGGAGAGTCCCAGGGACCGGG | |
| | | GATTGAAGTGTCTAACATTTAAAAATACTTCAATAGTTC | | AAGCCTGGCGGGCTGCCCTCTATGGGTTCGCACAGAGTT | |
| | | TATTATAATAGTTCAC | | GGACACGACTGAAGCA | |
| 345371 | SCAFFOLD | TGTTCCCAGAAGCAGCCCCCTTTCTCTATCCTGATGGAA | A/G | GTTATCGATTGCTGAATAATGATAATTATTTCTTTAAAA | 82 |
| | 11084_ | GATGTGAGTGGCCTCGGGTGCATCATTGGTATGCACACT | | AACAACATTCTCATCATCCAAAAACTATGAACATTAAAT | |
| | 11208 | CAGTGGTCTCTGGCTATGGAAAAGTAAGACTTCGAGCAC | | AATGGAGAGGATGTGGAGAAAGGGAACACTTTTGCACTG | |
| | | TCACCTTTGGTTTTCTGAGCCACGACTCCTGAAAGGCTT | | TTGGTGGTAATGTAAATGGATATAGTCAGTATGGAGAAC | |
| | | AGCAGAAAGCAGAGTCCTGGAACAGTGAAACAGAAGGGA | | AGTAGGGAGATTCCTTAGAAAACTAGGAATAAATTTACT | |
| | | GACACGTGAAATGGTTGAGGAAAGATTCCAGGATATTCC | | GTATGACCCAGAAACCCCACCCTGAGCAAAACCATAATT | |
| | | CCAAGGGCTGGGTATC | | CAAAAAGTCACATGTA | |
| 349676 | SCAFFOLD | AATTTACAGAAAGTGAAAAACCCCTACGTGATTGTTGAA | G/C | ATTGCTGAGTTGATTACTGGAGAGGAGATAGGAATTTTT | 83 |
| | 7023_ | ATAAATGTGACAGTGGAACTGATGATGCAAAATGCAAAA | | CCTAAGGAAGAAAGAATTACCTAAGAGGAAAGCTCAACT | |
| | 4826 | TTCTCATTTCTCCAAACCTTCTTCATATTTATGGACTAG | | CTTTCCTTTTTGAGGCTCAATCTTATAAACACCACTCTG | |
| | | ACAAGTCTTTCTGGGGCAAATATTTATTTTGAAAAGTGC | | ACAGACCAGATCTTTGAAAAGTCCAGTGACATAAAGGTC | |
| | | CACTTGAACAGACAAAATTAACTTATTACCCAGGTGAAA | | TCCAATGTCCCCAGGGAGGAACTCACTGTGGGAGAAGGT | |
| | | AAGGACAATACAAGCTTCTTTGGCTTAAAAAAAAGTTGC | | AGAATTGAACAGGTGAGCAAAGTGAAAGATTAAAGTGGA | |
| | | TGTTATCTGATTTTG | | AGATTGACTTGGGGAG | |
| 350777 | SCAFFOLD | CGATGATATAACTCTTTTATTGTATTAATGGGATATATT | T/A | AAAAAAAAAAAGTGAAATTGACGAACAGTTTTCCTTTAC | 84 |
| | 166086_ | TCCTAGTATTTGTTTATTTATTTCATTTGACTGTGCTGG | | TTGTCTGTATGTAAATCTGCATTCTGACTGCACAGAGAA | |
| | 6743 | GTTGCCGGATCTTCTATCTTCCTTTGGGCAGCTGGATCT | | CTCGGCTGTATCCGTAACTTTGGAGAAAGTCCCTGACCG | |
| | | TTAGTTGTGGCATGTGAACTCAGTAGTGGCATGTGGGAT | | GCTCTAGCTGCTTAAATTACCTCAGGTCCAAATCTTCCG | |
| | | CCAGCTCCCTGACCAGGGATTGAACCAGGCTCCCTGCTG | | CTGCCAGGTGGGCTGGACTCTCAGCCAATCCCAACCCGC | |
| | | GAAGTGTGTAATCTTAACCACTGCACCACCAACTAAGTC | | TGGTCGGGGAGAATAAAGGGAAACAAAGTTCTAGTGGTC | |
| | | CCCATTTCCCAGTATT | | AGCGTGGACTGTCTG | |
| 344359 | SCAFFOLD | TAAATAGTTCCAGTGCCGGGTTCTGGAGGGGAGTGTCCA | A/C | CAGTAACAGCATGGGGAACTTTTACATTTATATTTTGTC | 85 |
| | 55579_ | TCAGGTTTGGCCTGCTCAAAAAAATTTCATCTATGACCT | | CCAGTATAAGCTTATCTGCTTCTCTGACCAGGACCACAG | |
| | 11178 | CCTTGCAGTTATGATCGAGGGTTCCCACCTCTGTAGGTA | | TGGCAGCCAATGCCCAGAGACATGGCAGCCATCCCGTGG | |
| | | AGTAGGTAAGTAGGTGGTGGGATTCAGTGTCCGCACAGT | | CCACACAGTCCAGTTGTTTGGACAGGCATATGACCAGGC | |
| | | CTCGAGGTGGACCTGTGGGTTTTCACAAAGCAGTCCCTG | | GCTGCTATGGTCCAACTGTTTGCATGAGGACCCCCAGTG | |
| | | ACAGTGAGTCATCCTGGTATTGGTCAGTTACTTGTGCCC | | CAGTGTGATTTTTCTCATGCACAAATAAGTTAAAGTCCC | |
| | | TTGGCTGTTCATCAGC | | ATGTCACATCTGGCAG | |
| 345126 | SCAFFOLD | TGATTTCCTGGTAAAGCGTCTGCCCACAATTCGGGAGAC | G/A | TGGGTTGGGAAGATCCCTTGGAAAGGAAATGGCAACCCA | 86 |
| | 50281_ | CCGGGTTTGATCCCTGGATCGGGAAGATCCCCTGGAGAA | | CTCCAGTATCTTACCTGGAGAATTCCATGAACAGAGGAG | |
| | 6253 | GGAAACAGCAACCCACTCCAGTACTCTTGCTGCCTAGAA | | CCTCAAGGGCTATAGTCCATGGGGTCACAAAAGAGTCAG | |
| | | AATTCCATGGATGGAGGAGCCTGGTGGGCTACAGTCCAC | | ACACGACTTAGTGACTCAACGACAACAACATCATCAAAG | |
| | | GGGGTCGCAAAGAGTCGGACACGACTGAGCGACTTCACT | | CCTCGTGTACTCTGAATTTTTAAAAAACGTGTGCCAATA | |
| | | TCTTCAAAGCCATATCTGGGCTGGTAAAAGAATATGCCC | | CATTTGTTCAAGCCAGTTTGGCTTGCAACATTAAAATCC | |
| | | TCAGTGGATTTGATCC | | TCACTGATACATTGCT | |
| 347062 | IBISS4 | TTAATGTCAGCATAGTAAACTGTGTCGTTACTGAGGGGG | G/C | GGTAATTCAGTCTCCACTTTGTGTATTGTGTTGTCTTTA | 87 |
| | snp161 | AAGTGGTTGAT | | TACGCGAGGAA | |
| 348806 | SCAFFOLD | TGAGAAAGAGACCAAACCTTCCCTCACGTACAGCCTCTG | A/G | TTATATCCTGTGGCTTTTGATTTTCTGAAAATTGCACTT | 88 |
| | 7170_ | TGGGAGTCTGGGGTGTAAACCTCTTCGTCTGGTGTTGAG | | TCATAGTGTAGAGAAAAATTGTTAGGAAACCAGAAAAGG | |
| | 3304 | TTCTCTTCAGGCCACTGTAAACCTAATGGCAGGGAGTTC | | CAGGAGCAGCAGGCAGGCAGACGAGGACCCTCAGCTGAC | |
| | | CTTAAGGACAGTTTGGGGCTGCCAGCCTGGAGGGCAGGG | | GAGTGAGGATGGGGGATCGGATGGAGTGGGAATAGGGGG | |
| | | GGTGGTGACCTTGAACCTACCAGTGACAGGAGCAGCAGG | | GTCCACATATGGGGCCCTCCCCTCCTGCACACTCCCCCT | |
| | | TGTCAGCTTCTTACCCCTTTTGCATAAACAATTCATGAT | | TCCATTTCCATTTCTCAACACAGCAAACACTTCTGAGAA | |
| | | GGATTCACAGGGGTTG | | AATTAAAAGAGTCTTA | |
| 343136 | IBISS4 | ATCTTAATATTTGGCCTCTTTGAAGAGACAGCCCTTGCT | T/C | CTTTCCTACTGCCCTGGAATGGGTGTTGCCCTGAGGATG | 89 |
| | snp69 | GCTTT | | TATCCCCTCAA | |
| 348798 | SCAFFOLD | CTTCATATTTATGGACTAGACAAGTCTTTCTGGGGCAAA | A/G | TCTTATAAACACCACTCTGACAGACCAGATCTTTGAAAA | 90 |
| | 7023_ | TATTTATTTTGAAAAGTGCCACTTGAACAGACAAAATTA | | GTCCAGTGACATAAAGGTCTCCAATGTCCCCAGGGAGGA | |
| | 4924 | ACTTATTACCCAGGTGAAAAAGGACAATACAAGCTTCTT | | ACTCACTGTGGGAGAAGGTAGAATTGAACAGGTGAGCAA | |
| | | TGGCTTAAAAAAAAGTTGCTGTTAATCTGATTTTGGATT | | AGTGAAAGATTAAAGTGGAAGATTGACTTGGGGAGGCTT | |
| | | GCTGAGTTGATTACTGGAGAGGAGATAGGAATTTTTCCT | | CGACATGGGTTATAATTCAGTGAAGACCAGAAATACATA | |
| | | AAGGAAGAAAGAATTACCTAAGAGGAAAGCTCAACTCTT | | TAAACCCAACCATTTTGGCAAATTGGTTACGGAAGCAAG | |
| | | TCCTTTTTGAGGCTCA | | TAATCATTAAACTTGA | |
| 349583 | SCAFFOLD | GTCCAGATGCCTTGGCAATCCAGAATGCAGTCAAGAATT | A/G | CTACAGAATCTGTTACAGTATTATTACTGGTTATTAGAA | 91 |
| | 35711_ | AAGTAGGAAAAAATAATCTGAAAAACTCAGTGATTATTC | | AGTAGATTAAAACCTACCCAAAGTAGGCAATGGAACAAA | |
| | 6569 | AAAAGTCTGGTTACTAGTATTTATGTATTTACTAATACA | | AACATAGAAATCAGAATCTATTTAGCAGGATATCCTATA | |
| | | AGCCCTTGTTTAGCTCTGATGGGTAAAACCTTTCTTTTG | | CCTTAGTCATCTTCGGGGAAAATAATCACAGTATATCGC | |
| | | TAAGTTTTTAAAAAGTTGATATTTGGTTTCTCAATCCAC | | CTGACAAAAACATAGTAACCAAACAAATTTTAAATAAAT | |
| | | AGTACAATGGAAAAAGCCTTGAGGGGAGGGGCTAGGGTG | | GTTTGCTATATTAAAATAGAACAAAAAGGCTTAGCGTGT | |
| | | GAGGAAGATCTCAGAG | | GATTTTTCATGTTTTT | |
| 344029 | SCAFFOLD | TGTTTGTATGTGACCAAAGCATCATTAGTCCCACCACCG | A/G | AAGAGTGGTGTTCCTCCTGGCCTAAGATTCCGGGCGTTG | 92 |
| | 195103_ | TGAGACTGTTGCTATTTGCTACTATTCACTTCTGTTGCC | | CCTGGCAATGCTGCTGTGTACCCCAAGTTCCCCAGGGTG | |
| | 30086 | ACTGCTGCTGCTATCCAAACTGTCGCTGTCTTCAGTACT | | GGCCCTTTCCCTCCCTGCCACCAAGCTGGCTCAGCTTGT | |
| | | GCTGTCAGTTTCTATCTTCCCAGTTGCTTATAGTCTCTG | | ACAGTAAGCGTGTCACTGGTCTCCAGCTTGTGGCCAACT | |
| | | ACATCTGTCACTAGAAAGCTGGAACATACACAGCTCGTG | | ACACTCACATGTCTGCCACCCTGTCCACTTGAGGTCTTA | |
| | | AAGTAATTCGGATTGTGGCCGAAAGTACACTAGGAACCA | | CGCACCTCTTTCCCTAGCATACTGCGTAAAAACACTGTC | |
| | | GCACTGGAGGTACCAG | | TTAGCTCAGGCTGTGG | |

TABLE 7
DNA sequences for further SNPs identified in the analysis

| | | | | | |
|---|---|---|---|---|---|
| | V1 | | | | |
| | Scaffold | | Alter- | | |
| | or | | na- | | SEQ |
| | IBISS4 | | tive | | ID |
| Locus | No. | 5′ Flanking Sequence | Bases | 3′ Flanking Sequence | NO: |

| | | | | | |
|---|---|---|---|---|---|
| 344786 | SCAFFOLD | TTCACCTTCCTCCATTAGAATGTAAGCTCTATTTATCTG | A/C | TCCCTTTGTCCAACAGCCACCAATTCCAGCCAGTTTTTT | 93 |
| | 206105_ | TCTAGTTCCCTATTATGTTTATAGTACCTGGACCATTTC | | CTCATAACTGTTTCTCATATCTGCCTCCCAACTCCATAT | |
| | 7435 | TGGAACATGGTAGGTGCCCAAATATTGAGTGAATGAATA | | CTGTGATTACCACCTTGTTTCAGACCCTCAAACCTGTCT | |
| | | AATCTTCCTTCAACCCCAGACTGATTTCTATACCTTTTA | | CTGGGAAAAAGAATTCATTGTACTTTGTTGATGGGAATG | |
| | | TCTTGATGAGTGCCACCACCCCTATCCTCAGTCCCTTAC | | TAAAGTAGTACAGCCACCATGGAGAACAGAACGGAGGTT | |
| | | CTCAGAAACTTGGGTGTAATTCCAGAAACATCTGTCACT | | CCTCAAAAAACTAAAAATAGAATTACCATATGATTCAGC | |
| | | ATCACCACATCTCTGC | | TATTCCACTACTGGGC | |
| 347283 | SCAFFOLD | TTCCTCTGAACACATTTTATATGTTAATTCATCTTCTTA | A/G | GGCAAAGAATTCATCCCCATCCTTTTACAGATATCGTTC | 94 |
| | 170246_ | TGGATCTATTATTTCTAAAATTGCCAAAGCTAAAATAGT | | TTAAAAACAGAAAGTGTAATATGCTGAACATTTCCTATC | |
| | 10502 | GGCATTACATAATCTATGTTCCTAGGCCTATAACTCACC | | ACATTTCCCATCACACTGCAGATATTCAATTAGTATTTG | |
| | | AAAAATTTTAACCTAGAATTTACTATCATTGTTCAACTT | | TTAAAAATAATTCATTAAAATTAAATTCATTACTTTTCT | |
| | | AGCAATAAGTGGTCAGAAAGGCAAAGTGGCAGTCATACA | | TAATACTGATTCCCAATTAAGTTGTTTCGGAATTCTGTT | |
| | | AGTGAGTCTGTGTAAATGTTTGCGACATCGTAATTGTCT | | CTTAATTCTTTTCTTGAAGTGGGTCTCCTTGCAAGGAAT | |
| | | GTGCCAATTGAGAATC | | AAAGAAAGACAGAAAC | |
| 351702 | SCAFFOLD | TTAGTGGCAACTTAGCCTTTACTTTAAAAAAAAAAAAAA | A/G | TAGCTTCTGTCTATACAATTGATTTGTTCATTTTTTTAT | 95 |
| | 285370_ | AGGTTCCTGTAAGAAGAATGAGAGTTATAATATGCAAAT | | GAGATCAATATATTCTATAGATTGCAATTCATTTGTAAT | |
| | 4315 | ATATCATATTAATGGTATATTATATTTATTTGATGACAT | | GAAAAACTATAAATGTATTGCAGGCTGTGCCCCAAACAT | |
| | | CTTTTCCAATTAAATAGGAAGAATTACATTTGAACAAAT | | ACTTGCTCTATTGTCTGTACCTTCTTTTGTTCTTCCTTC | |
| | | CTCATAAATTAAACAATGCCTTCCTCTGTTTTGTTGAAA | | TCCCTGGATGTGTAGGTAAGGGATGTCACCTTACAGAGA | |
| | | TTTTTAATAATTTTATTTGATTAGTGGGTTACTATTTAA | | AATTCCTTCAGGGTCTTTGGAAAAATAGGAACTGAGTTA | |
| | | AGAATCCTCAAACACT | | TTTCACTCATAGCCTC | |
| 353387 | SCAFFOLD | CTAGGCAAGAACACTGGAGTGGGTTGCCATTTCCTTCTC | A/G | TGCAAACAAAGAAAATCTTCACACCTACTTGGAGAGTAA | 96 |
| | 76898_ | CAATGCATGAAAGTGAAAAATGAGAGTGAAATTGCTCAG | | TAGTGAAGATTAGCAGAAATCAAGGGTGAGAAATAAAGT | |
| | 3031 | TCATGTCTGACTCTTAGAGACCCTATGGACTGCAGCCCA | | AACATACTGCATGGTAATTGGGAGCTGTCACTAAATCTT | |
| | | CCAGGCTCCTCTGTCCATGGAATTCTCCGGGGAAGAGTA | | AGTTTCTCTCCTGTTCTTTCTTCCTTTTGTCACAAATAT | |
| | | ATGGAGTGGCGTGGCATTGCCTTCCCCGGTGTACCCAAC | | TCTTCAGGCACCCGATTTCTCTGGTGGCTGAGATGCTAA | |
| | | TATATATCCTTAAAAATATGTGACACAAGTAAAGACTCA | | AGCATCTGCCTACAATGCAGGAGACACGGGTTCAATCCT | |
| | | AATATTTGTTGAATAT | | TGGGTAGGAAGATCATC | |
| 345292 | IBISS4 | TTGGAGCTTTGGTGTTCCTGCTGATGTCATTTCCATCCA | T/C | CTGATCCACTCAGTGTTGTCACCAGATCCGATTTGTAAC | 97 |
| | snp168 | CATACCCCCGA | | CCACTGAGAGG | |
| 351344 | SCAFFOLD | GGGTTCTGTCTTTACTCTTTCATATTAGAAATCTTCCTT | C/T | AGTTACAGAGTTATACAGCTACACATACTCATTTGTACT | 98 |
| | 100386_ | TCTTAATGATTCAAGATTTTTTTCTTCTTTTCCCCAGGA | | TGTTTCCATATTATATAGATTTCCATATTATTTTTACTT | |
| | 1493 | CACATTAAAGTGCATGGCATGCTAAAGGTGTTCAGTATT | | CATAATTTATTCCAGAATCCATATTTCAAAATACATTGC | |
| | | TTCTGAATGTTAAATTCAGTTCAGTTCCCTTCAAGTCAC | | AAAATAAAAGATTACTTGTAAAAGTTTGATAATTAAGGG | |
| | | TCAGTCGTGTCCAACTCTTTGCTTACTTGCCCTTTAAAA | | CTTAAGTTTTAGTTGTTATTTATTTATAAATAATAATAT | |
| | | GATAGATATAGTCTTAGGTTTGCTTTTATATTCCTTAAC | | GTTATATATAAATGAGTAATCATTCACTCATTTAACATT | |
| | | ATCTTAATTGCATTTA | | CACTAATTTAGTAAC | |
| 350444 | SCAFFOLD | GGAATGCTCTTTTTACAGAAAATTTATGCTCTACACTTT | T/C | CAGAGGATCCACACTATTTGTGATTCAACCCCATATTCT | 99 |
| | 51917_ | ACAGGCACTGCATGTTAATGAAGGAAATAATGGCTAGAA | | GAATGAAGACTAAGCTGAGTTGGTAACTCTTATGACCTT | |
| | 2160 | GCTTATCATATGCCTCTGGGCTTGTGTCATGTTGCTTAC | | TGCGGGAACATGTATCCCTTAAGTGGATGAGGAAGTTAC | |
| | | AGGCATCATTCAAGCATCTACGTAGTCACTGGGTATACT | | CACATCTCCATATCCCTTGATTCTGTCTAGGTCAAGACT | |
| | | ACTCTATAATGATTGTTGACCACTAAACCACTTCATCAT | | ACCTAATCCTTTCTTTGTTTTCCCAGGTCTTGGTGGAGG | |
| | | CTGCAGCA CTTTTCTTACAGGGAGAACTTGCATGACTT | | TTGCACATAGCATCCTCCTTTCCAATATATTGTTCAGAA | |
| | | TTAATTAATGCGTGGTT | | TTAATTTTCTTAGGAA | |
| 346634 | SCAFFOLD | TCTTATGAACAAGGAGATCTACAGGAGCCAATCAGGGCA | C/T | GGCATGATTCCGAGTAGCTAAACTGCTCTCTGTAAGAAT | 100 |
| | 25012_ | GGCTATATAATGTGCAGGGCCCAGTGCAAACTGAAAGTC | | ATGACGTTCAAAGGCAGAAAGCTGGACTGTTTGACTTAC | |
| | 4993 | CAGGACCATCGTTCATTAATTCGAAAAATTCCAAAATAG | | CACCACATCCCTGAATACAGAATAGGTGCTCACGAAATA | |
| | | TGACAGCAGAGCATTAAAACAAACCCTTCTTCTAAGTAC | | TTTTTTGGCAAAGGGGGGAAATGAGGAAACGAACATAAA | |
| | | AGGCCCCTGTGGGACTGCACAGGGGACAGCCACATGGGG | | ATAATTTGTATGCAGTAAGTAGGTGCGTCATTACAGATT | |
| | | CCGATTCATTATTCGAATGTGGAGTGTAGAACCTCTTCT | | TGTGTCTGCTTTCAACCTTCCTCTGTAAGAAACTGACCC | |
| | | CATATGGAAATGGTTG | | ATTCATTTCAGTTCAG | |
| 346987 | SCAFFOLD | AATGGAGCATCTGATAGCATCTACTCCCTTTCTTGGTTC | A/G | ATAGATGACTAAAAGCAAAGGAAGCTTATAAAGAAAGCG | 101 |
| | 76322_ | AGATCATGAATGTGGCTCCTCTCAGCTCTCTAAGTCACC | | GGACATTTGCTTATGGATGGACAGGAAGGAGAGAAAGCA | |
| | 7745 | AGCTTTTGAGTATTTACAATCTGTGACAGTACAAAGAAA | | TCGTGCCCAGCAGCCACTTAGAGAAGCGTCTGCACAGGG | |
| | | TGGATTTTGAACTCTAAGCAGCCCATGGTAGAAGCAGGG | | ATTAAAAAAGGGCTTTCTTCAAGTTGAGAAGAAATAAGG | |
| | | AAGCCACAGCGGAAGCAAAACCAGAGTGGGTCCTGATAA | | AAGTTATTCTCTTGAAAAGGTCGTGGTAATTTTAAAGCT | |
| | | CAGTGAATTTTCTGTAAGTGGGAAGGAGTTATGAAAGGT | | GTGAGTGTGTGTATGCTCAGTTGTGTCTGACTCTTTTGT | |
| | | GGCAGAGAGGAGTGAC | | GACCCCCATGGACTGT | |
| 348902 | SCAFFOLD | GTCCACCTAAACCCATGTCCATCGAGTTGGTGATGCCAT | T/C | GAGTGTGCACTGATTGCAAAAAAGACTCTTAGTAACTCT | 102 |
| | 100431_ | CCAACCATCTCATCCTCTGTAGCATATGTATAAAATTTT | | TCTTAAAGTTAATGAATAGAGACCTCCTATGTGGTGTGA | |
| | 15454 | ATCATTGCTCAGGTTTGTTTGCTACCATTTTGAAGAGAA | | AAATATTCTTCCTACTTCAGTTCAGTTCAGTCGCTCAGT | |
| | | AAAAAGATGGATCTAAGATCCCATGAAAGACCATATCTT | | TGTGTATGACTCTTTGTGACCCCATGAATCGCAGCATGC | |
| | | GGTTAGCTAAAAGACCTAGGAAACCTCTTAGGATAGGTT | | CAGGCCTCCCTGTCCATCACCAACTCCCAGAGTTCACTC | |
| | | CTTTCATGAAGGAGTCAAAAAACTGTCAGTGATTAAAAA | | AGATTCATGTCCATCAAGTCAGTGATGCCATCTCATCCT | |
| | | AGAATAGAGAAAGTCA | | CTGTCTTCCCCTTCTC | |
| 349297 | SCAFFOLD | GTAAGCTGTAGTTTAACAAGCCCTCCAAGTGCTACTGGT | A/G | CATGTATGTTAGGAGTTTTTGGTGACTCTTCAAGGTGAG | 103 |
| | 230342_ | GTACAATGAGGTTCAAGTACTGTAACACATATTCTTAAC | | GGGTGGGTGGGCGATGGGTGTGATTTCTCTGCTTCTGAA | |
| | 10541 | TTTTAATTCCTTGGCTAGGAAGGTGCAACGAAACACCAT | | GGCACAGACAGGATCTTTTTGAAATAACCCAGAACTCAC | |
| | | ATTATACTTTGATCATCAACCAGTGATTTTATTAACTCT | | TGACTTTGGAAAGATAACGAGTAATACTGACACCGATGG | |
| | | TATCTTAAACTTTTTCTACACATTTTTTCCTGTTATTAG | | CTTTTATAAGCAATTCTTAGATGCATTCAATTCTGGGGA | |
| | | AAACATAACATTACTGATTTATCCTGTGGTCTTTAAACG | | ATCCTCTTTGCCCTAGAAGAATTGAGACTGTGATACACT | |
| | | TGTTTTTTCACCCAGAC | | GAGTTTATAATCTGGG | |
| 347122 | SCAFFOLD | TAGAGATGATTGAAACTATACAGCAGGATGCACGTTATA | C/A | CACTGGTAGTCTTAGAACAATGTAACAAAATCCACCATC | 104 |
| | 11052_ | TGTAAATACTATATCACTGTATCCAACGGACATGAGCAT | | TCCACAAAATAAAATTCAAATGTCCACTTCACATACCAG | |
| | 910 | CCACGAATTTTGGTATCCATAGGGCCTGGAACCAATCCC | | AATTACTTGACATATGAAGATCCAGGAAAATAAAACCCA | |
| | | CCACAGACACCAAGGGACAACTCTACTTTTTAAAAGACT | | ACCCCAGGAGAAAAGTCAGATGCTAGTTCCAAGGTCAAC | |
| | | ATATCACCAATGTATGCATCACATTCTCAGTTAATGAGA | | CAGATGCTGAAGTTACCATCAGGTAGAGACTTTAAAGTA | |
| | | ATTAGGACTGCAACATATGAATTTGAGGAGAACATAACT | | GAGCTTATAACTGGGCACAGTGAGACAAAGAAAAATGTA | |
| | | CAGACCACTGGCTAAA | | CCTACAAGGAACTGGA | |
| 349965 | SCAFFOLD | AATTGCCAAAGCTAAAATAGTGGCATTACATAATCTATG | A/G | ATATGCTGAACATTTCCTATCACATTTCCCATCACACTG | 105 |
| | 170246_ | TTCCTAGGCCTATAACTCACCAAAAATTTTAACCTAGAA | | CAGATATTCAATTAGTATTTGTTAAAAATAATTCATTAA | |
| | 10559 | TTTACTATCATTGTTCAACTTAGCAATAAGTGGTCAGAA | | AATTAAATTCATTACTTTTCTTAATACTGATTCCCAATT | |
| | | AGGCAAAGTGGCAGTCATACAAGTGAGTCTGTGTAAATG | | AAGTTGTTTCGGAATTCTGTTCTTAATTCTTTTCTTGAA | |
| | | TTTGCGACATCGTAATTGTCTGTGCCAATTGAGAATCAG | | GTGGGTCTCCTTGCAAGGAATAAAGAAAGACAGAAACAG | |
| | | GCAAAGAATTCATCCCCATCCTTTTACAGATATCGTTCT | | TGTGAGCACATTATAGCTAAGGCTTCCCAAGTGTTGCTA | |
| | | TAAAAACAGAAAGTGT | | GAGGTAAAGAACCCGC | |
| 345591 | SCAFFOLD | CACAGCCAGAGAAAACACCCCAAAATCGTTTTCCAGGGC | G/A | TGCTATTTCTTCCTGACTCCCTCAGAGGACCCTGGTGGA | 106 |
| | 190194_ | ATCTGTGCCCTTGTGGAGGCTGCCACGATGTTTTAATTG | | AATGTGTCTAGAGGGGTTCTGTTGGTGGAAGGCATCCAG | |
| | 25419 | AGTTGGATTGAATTGCTACGGAGGGAGCAGAAGCAGTGC | | GGAGGTTGTCCAGATTGGGGAGCCTGTGCGAGAAGCCAC | |
| | | ATTTTCAAGGACTAATAATTCTGTAATTTTGGAGGCGGG | | GTCCAGCATCATCGAGTCCTTGGTTACACACTCAGCTTT | |
| | | GCATACATTTCTTGGCACAAATAGGCAAAAGGCATCTGC | | CTCGTTGGTATCTGCACACTTCACTCCTGGGTCACTGGC | |
| | | ATTACTCTACAGCAGGCATTCTCATTTGGAACAAGACCA | | TTCTGCTGAATCCACCCAGTTCTCCCAGGAGCTGACCAC | |
| | | GGACTGTCAAAAGTGC | | GTGAAAGGGTCGATACA | |
| 347733 | SCAFFOLD | TTCAGGCAAAGGGAAGAACATGTGTAGCTACCCTGTGGT | T/C | ATATACTCATATTCAGCCTTCACTCAAGCTGTACTTAGG | 107 |
| | 316970_ | GGGAACCAGCTTAGCCATCAGATCAAGCAGCAGAAAGGA | | TTCTTCAAGATGCCCTCTTTTAGCAGTGTTATAAACACT | |
| | 12453 | GGTGCCTATTGCTGGAGTGGATGCGAAGGAGGAGGAGAG | | AAAATTGTTTTAAAAACATGCAGATACCCATATGTAACT | |
| | | TCCAGTAGACAGCCATCTAGATCTCTGCTCATCACATGG | | ATTGCAAAAATTTTTTTTCCTGAATTGCTCTCCTGGAAT | |
| | | GATCCCAGTAACAACTCTTCTCTTGCATCCAGAAAGTTT | | TCTAGCACCATGTTCCCTCACACTTCTGTCCCTTTACTC | |
| | | AAAGATTCTCAGGGTCGGAAGGGACATTTCCCAAATGCT | | AGTGTTTTCTCTACTTGAAACTCCTTCTTCCAACTTATT | |
| | | CCCAGTCTCTCTTCTC | | CTTTGTGTTTTTTTAT | |
| 350995 | SCAFFOLD | GACCTTCAAGAGAAGCCGATTGTTTACAGGATAAAACCC | C/T | CCTGTGTTATGCTTTCTTTGATCACTCCCACCTTCCCCA | 108 |
| | 265171_ | AGATTCTATAACAGAGAATGAGTCTCTTCACATCAGGCC | | AGCTTCCTTCCCATGAGTTGACTATGTTCTTGTGTCACT | |
| | 9413 | CCATAAGCATCTGTTACCTGATGTTCTCCTACATTTAAT | | ATCTCTAACACATATCCTTATCAAATACTGTAATACGAT | |
| | | CTTATGAAACTATTCTTCCCAGAATGAGTAATATTGTTC | | TATGTACTGACATCTTGTCTCCCCCACTGTGACACCGAG | |
| | | CCTTCAACTCCATGCCTTTGCAAAATTCTCTTATTTTTA | | AAGGGCCTGGTGGGACTCCTGGGCACAAAGCCTTTCTGT | |
| | | AAGAATGCTCTTCCCTGTTTGATCTGCTTAGCCAATACC | | GCCCATGACCTTCCCTGAGTTCCAAGGGGGAAGATACAA | |
| | | TACTCATTTTTTAAGA | | CCAGTTGTTAGTTAGA | |
| 345261 | SCAFFOLD | ATTGATATCAAGAAGTATTTAATTGGGGAATGTGGTAGA | T/C | GGTCATATCCAACTCTTTGCTACCCCACGGACTGTAGCC | 109 |
| | 95041_ | GGTCTGTGTTGTCAGATTGACAGATTAGGATTATATCTA | | TACCAGGCTTCTCCAGCCATGGGATTTCCCAGACAAGAG | |
| | 43584 | GATGGGATATTTAGAATGCAGGATCACAAATGCCCCTTC | | TACGGGAGTGGTTTGCCATTCCTTCTCCAGGGGATCTTC | |
| | | CATTTCTAAACTCGGTAGTCCCCCATTTTGCTCTTCACG | | CAGATCCAGGGATTGAACCCGGGTCTCCTGCATCGCAGG | |
| | | CACTTAGAATCGGGCCTCTGTAACCCAACTGAACTCATT | | CAGATGCTTTACCATCTGAGCCACCAGGGAAGCAGGATA | |
| | | GCTGCTTGTGTTCTCTCGATTGTCGGGATACTCGGAAAC | | CTCTAGAAATTATTAATAATTGAAATATATTGAATGCTT | |
| | | TGGGACTGAAGTCGCT | | CTAATATAAAAAGTC | |
| 343500 | SCAFFOLD | AATGTTCTAAAGTTAAATGTTATTTAAGAAAGCAGGAAC | C/G | CTTCATCAAATCGCTCCAAAAAAAGAGTTTTTCCCTGAT | 110 |
| | 256107_ | ACAGAGCCACGTTCCTCATATACTGTTGTCTTTCTTCTT | | TATTCCAGTTCACCACTTATTTACTCTTCAACTCTTTCC | |
| | 8432 | CACTGCTTACAAACACATTCACAATCCTTCTCAATCACG | | TAAGTGCCCCAACCATTTGCCTGAAATGCCCTTCGCTGG | |
| | | AAACACCCAAGATCTCAAAAACTCAGACAATAGGCATTT | | TTCCTTGTAACTTCCTCACCTCTCACTACAAAGATTCCT | |
| | | CCCAGCCCCAAGAAATGCAGTCGAGCAGGGAAGACACAC | | CTCCCTGCCCGCTCTAGATGAGTAAAGGCTGATCACGCT | |
| | | AAGAAAATGAGAGCTCAGCTAGGGAACCATCAGAGTACA | | TCCTTTTTTTCTGAAATTCTCTTTTGTTATATTCAGTTC | |
| | | GAAGAGGGTGCCCAGT | | AACTTCAGTTTAACAA | |
| 346106 | SCAFFOLD | CCAAATAAAAAATCACAAGCCTCATTATCACAGTATTGA | T/C | GCAAGCCTAGCACTTAGTGAAACCCTGAGCATCCCACAA | 111 |
| | 70728_ | TTATATGGCAATAACAACCCTCACCATGAGTGAAAGCTT | | TTGACCCACCTGGCTACACACAGCTTTGAGGAACAGACA | |
| | 7857 | ACACTATTAAAGGATTATGAGCTTATCTGGATATTTTTT | | GAATTATTAGTACAAATAATTCCCCTATCTCTTGATGAG | |
| | | AGCATGAGTCACAGGACAAACATTTGGAAGAGGAAAGCA | | TTATGTCCTCTATCACAGTAGCCAGGAACGCTTTCGCTT | |
| | | CCATATATAGAAAGCAAACCTGTTGCTTTTTGTCGGTGC | | GCATCAGAAAGCAGGCTCGAATCAGCTGCCCTTGGGCGG | |
| | | TGGCACAGCCCCAGCTGGCCACTAGGTGGCAGTAGTGTC | | AATCTGGTCAGCAGAGATGTGTTTGCCTAGAGCAGTGTT | |
| | | CTAGCAACAGTAAAAC | | TGCTTTTGTTATTTTA | |
| 346573 | SCAFFOLD | CAGTCTCAAGACTCCCCCAGATCTACTGAGTTGGATCTT | A/G | CTTGAAGTCTTAGTTCAAACATCTGTAGTTCCACAGCTT | 112 |
| | 226597_ | GCGTTTTAATAAGATCCTCAGATCATCAATAAGATTCCC | | TCCAGGATGGCCACTGGCTCCCCTGCCCCACTCAGCATC | |
| | 2085 | AGATCCCCTTGGCTAAAAGGTGACTGCACAAAGCCCTGT | | CACTACTGCCCTGTGCCCTCGGCAGGGACCTTGAACATT | |
| | | CTCAAAAGGGGTGCCCCATGCTTGGGGTATAATGCTCTG | | GGTATGGTGACGTCAAGGTCAGATACGAATGCCCCATGT | |
| | | TGGTGACTGACTTGACATTCTTGCTAATTTTATCTTTGA | | GCTCAATCATGAGTGATGCTTTGGGCACCTTTGAAGGCC | |
| | | AATTATATTTTGTGAGTGAAATCTGGTGGGACACTGAAA | | TGTTTCTCTGTCGGCATCCCTGTATGAAAGCAGCACAGT | |
| | | CTTGTGAGGGAAGCGG | | GTTAAATGGCAAATAA | |
| 353748 | SCAFFOLD | TTTTAAAAGATGACACTTTGATTTTAGATTCATCAGTCT | G/A | TCCAGATTAAATGTATATAAACTTAAACTGAATTGATTG | 113 |
| | 115426_ | CATCTCACCGCCCCCATAAAATAATCAACCATTCAGTCA | | ATACAAAGATAAATATGATTTAGCCCCCTTTCTGAAGGA | |
| | 13127 | ACAAATATTAGGCATGATTGTAAACATGTGAGAGAAAAT | | GCTTATACCTTAAGTGTTGAAAGAGATATATACAACTTA | |
| | | TAATTTAATTTAACAAATATTTACAGAGCAGTGCCAGTG | | CTGGAATGCCAGGCAAAATAAATCTTGTAGTCGGAGCAT | |
| | | GAAGAGATACAGCAGTTAACAATAATTGACTGTCTGCCT | | AAAAATAGTACTTTGGGGCAAAGATTAGGAAATGATTAA | |
| | | CTTTGAATTTACAGTCTGTCTGAAAACAAACAGTAAACA | | TTGGGACCTCAGGAATTGCAAAAAAACTCGGTGAATAAG | |
| | | GGTAATTACAAAGGTG | | AAGTCAGCCTAGAAGG | |
| 353932 | SCAFFOLD | AGGCAGATTCTTTACTGTCTGAGCCACCAGGGAAGCCTT | A/G | CAGTGATCATTTAAATACATTTTCCTACATTTTGCAGTG | 114 |
| | 76928_ | ACATTCAAGCAGAGGGACTGGAAAAAGCAAAGATCTGAG | | CTAAATTCATCTATAACAGAAGGATAATTTAATTCCAAA | |
| | 9451 | ACCAAAATAGGAAAGTTGGTGGGGCTGGAGCCCAGCCCA | | GGCAAATTAACCGCTCTTTGCCTTCTCCCAAAGTCACCC | |
| | | TGGGGACAAGGACTGGAAAAGCAAGTGGAGGACCAGATC | | ACTTTTGATATTTTTTTCCATAACTTGATTCAATTTTTT | |
| | | TTACTGCAGACTGTAGTAAAACACTATTTATAGGTTCAA | | CTGCTCTTGCCACATTTTCTCCAGCTCCTACACACTTTT | |
| | | ACAAAAGAGCTAAAATATCTCAGTAAGATCAAAATGATT | | TTGGATGGTTATAACGTTATTATCTTATTATTAGTTGCT | |
| | | TATTGGATTTATTAGC | | AACTGGGGATAATTCA | |
| 345696 | SCAFFOLD | AAATAAAAAATATCATGGTAAGTCAAAAGATGGGAGAAG | T/G | CGATTGAGCGTGAGTATAAATATAAGAGAAAGAGCATGG | 115 |
| | 226597_ | AAAGGAAAATGCTATTTTCCTGCTCTTTGGACAAGGAAT | | GCATGATCAGAAGACCCACATCTCTCTCTCGTTCATGTT | |
| | 1590 | CCAGCATTTTCGTTTTGGTCCTTGTAAGTTATGCTCATT | | TTGCTGGTATGTGACGTTGGGCAAGTCACTAGACCTCCT | |
| | | GAAATTTGACAAGCTTTAGTATAAGAGATGGAGAAGGAA | | TGCCTCAGTTTCCTCATCTGTAAAATGAGGGTGATGTCT | |
| | | ATAGCAACCCACTCCAATATTCTTGCCTGGAAATCCCAT | | TATGCCCCTTTCCAAATGGGCTGGGTCTAGGTTAAGGGC | |
| | | GGACAGAGGAGCCTGGAGGACTGCAATTGGTTGCAAAAG | | AGACCTGGCAACATGTCTTTGAGTAGGAATGTGTCCAGA | |
| | | ATTCAGGCAGGACTTA | | ACACCTCTCATCTTTG | |
| 344027 | SCAFFOLD | CTCCAAACATATACTGGGGTTTGCAAGAGGCCTCTGTAC | A/G | TCCCAGGTATCGTCCCAAGTGCTGGGAGTATAAGGAGGA | 116 |
| | 192022_ | TGACTGGGGTGCAGTGGGGTGAAGCCTTCCCTTTCTGAA | | GCACTCCTCAGTTCTGGCCCCCATCCCACCACTGGCCTT | |
| | 4174 | GGTCAGCCAGCATCACAGGGGTAATGCCCTCCTTTTGCT | | CACGAAAGAGAGGGCCGGGAAGTGGCACTGTTTAATCTC | |
| | | TAGGCCCTTACTTTTTGTGGATTTCTGGCAGTACAGATC | | TGGGGGAAGCCCATCCTGCAGCTTGCTAACAGATCTGAG | |
| | | ACAGCAGCCTGGGAAATACAGATTATCTGCTTTGTGGAT | | ACTGCCTGAGGACAGGCAGAGCTTGAAACCACCGAGCGT | |
| | | TACCTGGAGCTCTAAACCTAAAGTTTAACCAGCATTCAG | | AAGGAGCTGTGAGTTAAGGTATCTGTCGCTGAGTATTAG | |
| | | TGTGTGCCCATGAT | | CTATGCTGTGGATGAC | |
| 345464 | SCAFFOLD | AGTTAAGTTTTCAGTCTTATGAAAGCAGCCTCAATGAGT | C/T | GGTGGTTGCTATTTCCAGCTCATGTTTATGAAATACATA | 117 |
| | 150213_ | GTTTCTGCCTGGCAGTCTCAGTTGCCACACTGCTTTGCC | | TAATTTTAAGGCTTTGCCAAGAGAGAGCCTTCCCTCTGT | |
| | 25683 | AAATTATAGTTTATCTGCACTGTGTTTTCTGGTCACGGG | | GGTTGATTTTCAGAGTCATAATTACCCTCTTATTACAAG | |
| | | CTTCAGCTGAGCAGGACGAGACTCTGAGGACTGAGACCC | | TCAGTGTTCTGGGCCCTGACTCCCCTGGGTTCTTGTGGC | |
| | | ACATATTCAGACCATGGGTGCTCCCTGGGCTGCTGCCGA | | CGGCTCGTGGGACAGACAGAGGTATAACACTGGTCAAAT | |
| | | CCCACTGGGTCTGGAAGCACAGGGGTCACAGTGGGACTA | | CTGCTGACCACGCTGTTCCGAGCTAGCAGACTCGGCAGC | |
| | | GGCTAGTTCTTTTCCA | | CTTGTCAAGGGTTTGA | |
| 352784 | SCAFFOLD | TGCTTCCATGTATTGTGATAATGCTGCTATGAACATAAG | T/A | AAAATAGTAGCCATCGTATATGTTCATGTATAACTGATT | 118 |
| | 260109_ | TATACAAATATATCTTTGAGAACCAGCTTTTAGTTGTTT | | CACTTTGCTGTACAGAAGAAAGTAACACAACATTTTAAA | |
| | 14985 | GTAATATATACCCAGAACTGGGATTACTGAATCACAAGT | | TCAACTATACTCCAACAAAAATTAAAAACAACAAAAAAA | |
| | | TAATTATAATTTTATTTTTTTTAACTCTGATATTGTTTC | | CAGTGGCCATCCTAATGGGAATAGCTTTGATTTGCATTT | |
| | | ATAGCTGCTGTAGCATTGTACCTTCCCACCAATGATGCA | | TTCTAATGTTAAACATCTTTTCACATGCTTAGCCATTTT | |
| | | CAAGGGTTCCAATCTCTCCACATTCTCATCAACACTTAT | | TATATCTTTTTTAGAGAAATGCCTATCTAATTCCTTTGA | |
| | | TTTCTCTCTTTTTT | | CCATTTTTAAATTGCA | |
| 346642 | SCAFFOLD | TGCCTATGTGGCTCATATCTGTGGCTTCTGTTTTATTTC | C/A | TTGGTGATGAGGGCTGTAATGGCGGAACATGAACAGGTG | 119 |
| | 251761_ | CAGTGGGTGGTGCTGGTCTAAGAGGTAAGGAGCCAGATA | | GGGGCGCTCAAGAGAAAGCGGTCACGCCGCCCCATAAGG | |
| | 4653 | ATAACAATCACGTGTGTAGCATGCTCTACCCATGCTCTA | | CGAAGCAAACCGTCCAGATGGAGATGACTGATGCTTAGG | |
| | | CACCGGACCCAAGTCATTTTATTCAACCCTCATAGTAAA | | CTGAGTGAGCCTGGAATAAAGACATCCACGTAGCCAACA | |
| | | CTCATGCAGTGGGGGTCATTATCCCTATTACAGATAAGG | | AGCAGAAGGGGTTTCCTGAGCAGAGGAAGAGCAGGTGTG | |
| | | CAGTGACCCACGAGTGAAACCACCGGCCCAAAGTCTCAA | | AAGTCTCAGAAGCATGAAACTGCATGAAGTGTCCAGGGA | |
| | | AGTGAACAAAGTCCCC | | ATGGCCGAGGAGGAGA | |
| 343918 | SCAFFOLD | CTTTCAGCCAAAATATCAATGAAGATACTACGTGACTGA | A/G | GCCAGACCAGTGAAAGGAGACTTGGAACGCAGCTCTGCT | 120 |
| | 144174_ | GGTTGAAGGTCTGGATATTATCATTCTTTTTATGAAACA | | CTGTTTTCTCAGCCAAGTAGCCTTGCAAAGCAAGTCACA | |
| | 3812 | GATTAACTATGTTTCTAATAACATAATTTTAATAACAAA | | TGACCTCCCTAGGCTTCAGTATCCAGATGTACAAAATGG | |
| | | CTGATTAAATAGTATTAGAAGATTCATAGTTCTAGCACA | | TGATAAGATCAGCATGCCTCCATGCCTCAGCTTGTTGTG | |
| | | GAAATGCATACCTGTCAAAATGCTGCACTGTCACTTTTA | | AGAAGCACTGGCAGGGGAGGCTGAAGGGAAAGACAGCAG | |
| | | AGCCTAGTTTATTTTCATACATGTGTATAAAACAGTGCA | | ATATGGAGAAACCTGGGCACTGGGCCCCTCTGAAGGTAC | |
| | | GTGTGATGGAAAGAC | | TCCCCCGTGCCCTGAA | |
| 346964 | SCAFFOLD | TTTTAAAACCAGAACAGACCACTCAGAAAAGGTAAAAGT | A/G | TACAAGCTTCTTTGGCTTAAAAAAAAGTTGCTGTTAATC | 121 |
| | 7023_ | ATAAGAGAGAATTTACAGAAAGTGAAAAACCCCTACGTG | | TGATTTTGGATTGCTGAGTTGATTACTGGAGAGGAGATA | |
| | 4778 | ATTGTTGAAATAAATGTGACAGTGGAACTGATGATGCAA | | GGAATTTTTCCTAAGGAAGAAAGAATTACCTAAGAGGAA | |
| | | AATGCAAAATTCTCATTTCTCCAAACCTTCTTCATATTA | | AGCTCAACTCTTTCCTTTTTGAGGCTCAATCTTATAAAC | |
| | | TGGACTAGACAAGTCTTTCTGGGGCAAATATTTATTTTG | | ACCACTCTGACAGACCAGATCTTTGAAAAGTCCAGTGAC | |
| | | AAAAGTGCCACTTGAACAGACAAAATTAACTTATTACCC | | ATAAAGGTCTCCAATGTCCCCAGGGAGGAACTCACTGTG | |
| | | AGGTGAAAAAGGACA | | GGAGAAGGTAGAATTG | |
| 342614 | SCAFFOLD | ATTTTTGGAGGGTAAGGACCCCGGTTTGTACCTTGACAT | T/C | GCAGAGGAAGGACTGCTCCCACAGACAGTGACAGTGGAA | 122 |
| | 371850_ | AATAAATGCACAAAGTTTTGTGCATATTTCTTACACAAA | | AGTGGCCCAGCCAGTGGGAATAGCTGGGGCATCTTGTTT | |
| | 156 | GCTTTTGCCTCTGCTGGTTGACTGACCATGTGCTGTACT | | GAGGCTGTCTGACTGGCTCCTTGTCATAACCCCAAGTTG | |
| | | AAAGATGACCTGCTTGGGGAAAGGATGCCACAGCATGA | | AAGGACTCTCCACTTTGACTCATCAAAGCCTGGAAATTT | |
| | | | | TTCTCAACTAGATCTGTGGTTGTGAAGATTTCCTGCCAC | |
| | | | | TACCTGTCTGAAAGATTCTCTCCTCATTTTTCATATTCA | |
| | | | | AGGATATGTCTCTATG | |
| 347425 | SCAFFOLD | TCTGCTCAGGGGCCCCACGACTTGATGAGCAGAGTGGAG | G/A | CTCCACATCTGGAGTCTTGCCGCAGCCCACAAAGCTCTC | 123 |
| | 216862_ | ATGGAGGAGGACTCGGGGCACGGCAGCGCATCTGCCGGC | | ATCCCCAAATGCAAAGCGTTTTACAAAAGAAGGGATTCC | |
| | 6835 | TCCGAGGAGGCATCCAGCACCCCCGAGACAAGCAGTCAC | | TTTAAATCCCAGTGTCCTTGCAGAGTCGGTGAAAGCCCA | |
| | | CCCAGCACTGACCGGGTGGCAAGCTCCCCCGAAGACAAA | | GAGTGCGTCAGCTACAGTGGATGTAGCTGTGAAAATTAA | |
| | | TTTTCCCAAGAAAATATGGAGTCTCGGGAAAAGTTACCT | | CAAGAAAATAGTGCCCCTTGATTTTTCCATGAGTTCTTT | |
| | | GAAACTGACCATCGTCTCTCAGGCACAAAGTGCCATTTA | | AGCCAAACGAATAAAGCAGTTGTGTCAGCAAGAACAGCA | |
| | | AACCAAGAAGAAAGCA | | ACAAGAAAGTCAACAG | |
| 352451 | SCAFFOLD | ATCCCATATGTCTTGAAGCAACTAAGGCTATACATCACA | T/C | AAACAAATATTCAAAAAAAAAAAAAAACACAAAAAAGTA | 124 |
| | 718_ | ACTACTGAGCATGTGCTGTAGAGCCTTCCTGCCTTAACT | | GATAAAGGAGAATAGTCATTTATTTATTTATTTGGATAG | |
| | 5757 | ACTTAACTACTGAGCCCATGCATTGCAACTACTGAAGCC | | ATTAAATAAGCTAATGTATTGGAGATATACTACTAGAAA | |
| | | CACACCTAGAGCCTGTGCTCCGCAGTAAGAGGAGCCACC | | GGTTACTAAAATTTGCCAAAATACTATTCAATGTTTGCT | |
| | | AGAATAAGAGGCCCGTGCACCACAGGAAGGTAGCCTCCA | | GTGTTGACTGAAGAGGCTAAGCAGCATTAAAAAACAAAC | |
| | | CTTGCCACAACTAGAGAAAGCCTGTGTGTAGCAACAACT | | AAAGCTTCAATACAGTTGACAATTAAGCATTTACCTTGA | |
| | | TACCACAGCCCCCAAA | | GAAACTAGTGGAATTT | |
| 344675 | SCAFFOLD | TGACTCATTGGAAAAGACCCGGATGCTGGGAAAGATTGA | A/G | TAATCTCCAGCATCCCCCACTTCTCCATAGTCAAGCTGA | 125 |
| | 155700_ | AGGCAGGAGGAGAAGGGGATGACAGAGGATGAGATGGTT | | TTTTCCTGGGGCTGCAGACCTTGGGCCAGGTTGGTGGGA | |
| | 8753 | GGTTGGCACCACTGACTCGATGGACATGAGTTTGAGAAA | | GTGGTAGTTCTACCTCTAGATGTCTCACGGCTTCCATTT | |
| | | ATTCTGGGAAATGGTGAAGGACACGGGGAAGCTGATGTT | | TCTCTCCTATAAAATGCAAAGATTGGTTAAATGATCTGT | |
| | | CTGCAATCCATTGGGTCACGAAGAGTCAGACACGACTGA | | AAATTCCCTCCAGGTTCAAAATTTTGATTCTGATTTGTC | |
| | | GCAACTAAACTAAACTGATCTGAACTACTGAAGACGCCG | | ATCTTTGGCATCAGGATAGCTCTGCCATTTAAATAGAGC | |
| | | TATGTCATCCATGTTC | | CCTTTTAATTAGTGCA | |
| 344550 | SCAFFOLD | CATCACTTCCATTAGCAATGAGGAGACTAGGCTGGGGTT | C/T | GCCTGGCACATACTACCTCGAGTACGTTGAAGCCCTTAG | 126 |
| | 10204_ | AACCAATGGGTGTGTTTTCAGAAAAGGTATCAGAACCAG | | CTGGTAGTATGAGAAAAAGTACAAGAAATATTTATTGAT | |
| | 22479 | GATTGAAGCAGTCTGCTCTCACTCTATACACTGCATGCT | | GACCTACAAGCTAGGTGATAGAAGAGGATGGGCATAGCT | |
| | | TTACATGTGGAAAAGAACAAGTGTACCTCGGGAGAGGCT | | GATACAGATTATGGATACAAAGAACTTACTCAGGCTTAA | |
| | | CCAGAATGAGAAGACACACTGCAAGCAAGTCGCCTGAGC | | AAGCCAGATGTAAAACCAACTTAACTATTTAATCATCTT | |
| | | ACAGTATAGGAATCTGCTGTTCTCCTGTGGAAGAAAGAA | | CCATCTCCCGCACTGAACTCCCTTAGGCCAGGAGGATGT | |
| | | TGAAGGCATTGCAAT | | TACACGGAAAATGAGT | |
| 346191 | IBISS4 | AGCAGGAAGACCTGCGTTTAATCGGGGGTGGGGGTGGGA | A/G | TATGGACTGTTCTTCCTGGGCCAATTGCCACGGAGGACT | 127 |
| | snp482 | GTCAGTTTGGA | | GTCATTGACGA | |
| 344923 | SCAFFOLD | GTGTGTAGCATGCTCTACCCATGCTCTACACCGGACCCA | C/A | GTCCAGATGGAGATGACTGATGCTTAGGCTGAGTGAGCC | 128 |
| | 251761_ | AGTCATTTTATTCAACCCTCATAGTAAACTCATGCAGTG | | TGGAATAAAGACATCCACGTAGCCAACAAGCAGAAGGGG | |
| | 4564 | GGGGTCATTATCCCTATTACAGATAAGGCAGTGACCCAC | | TTTCCTGAGCAGAGGAAGAGCAGGTGTGAAGTCTCAGAA | |
| | | GAGTGAAACCACCGGCCCAAAGTCTCAAAGTGAACAAAG | | GCATGAAACTGCATGAAGTGTCCAGGGAATGGCCGAGGA | |
| | | TCCCCCTTGGTGATGAGGGCTGTAATGGCGGAACATGAA | | GGAGACAGCTTGAGCTCCAGGAGCAAGTGGAGAGAGATG | |
| | | CAGGTGGGGGCGCTCAAGAGAAAGCGGTCACGCCGCCCC | | AGTCGGCAGAGATAAGCAGGAGCTTTGCAGGGAGAGGCC | |
| | | ATAAGGCGAAGCAAAC | | TGCATGCCACACACAG | |
| 353129 | SCAFFOLD | TTCCTAGGCCTATAACTCACCAAAAATTTTAACCTAGAA | G/A | CAGATATTCAATTAGTATTTGTTAAAAATAATTCATTAA | 129 |
| | 170246_ | TTTACTATCATTGTTCAACTTAGCAATAAGTGGTCAGAA | | AATTAAATTCATTACTTTTCTTAATACTGATTCCCAATT | |
| | 10598 | AGGCAAAGTGGCAGTCATACAAGTGAGTCTGTGTAAATG | | AAGTTGTTTCGGAATTCTGTTCTTAATTCTTTTCTTGAA | |
| | | TTTGCGACATCGTAATTGTCTGTGCCAATTGAGAATCAG | | GTGGGTCTCCTTGCAAGGAATAAAGAAAGACAGAAACAG | |
| | | GCAAAGAATTCATCCCCATCCTTTTACAGATATCGTTCT | | TGTGAGCACATTATAGCTAAGGCTTCCCAAGTGTTGCTA | |
| | | TAAAAACAGAAAGTGTAATATGCTGAACATTTCCTATCA | | GAGGTAAAGAACCCGCCTGCCTAGGCTGGAGACATAAGA | |
| | | CATTTCCCATCACACT | | GATGAGGGTTCGATCC | |
| 350612 | SCAFFOLD | TTGTTCAAGGGACAACTGTGATACAATTCCAACCCCCAA | A/G | TGTGGCATTACATTGTGGATATTCCACAACTTTTATATT | 130 |
| | 100457_ | AATTCCCTCTCCCTCTTTCCTATCAATTCGTAATCCAAT | | CATTTACCAGTGGGTTCCAGTTTGGAGCTTTATGAATAA | |
| | 11884 | CCCTGGTAACCTGGAACTGATTTCTGCCCTTATACTTTT | | ATCTGATATTAATGTTTACATACTGGTCTTCATGGTTGA | |
| | | CCTTTTTCAGAATATCATATACATGGAATCATATAGTAT | | GCCACTTTTGATGTTTTGATTTTTTTTTTTTCCCCCTAA | |
| | | ACAACAGTCTTTTGTGCTGGATTTGATGTTAAGCTTTGG | | CTTCACAGTTAATGTCCCAAGCAGTGGAACATTAGTGGT | |
| | | GGCCTCAGTCTTGCTATTACTGATATCAGCAGATTTTTC | | CTTGCAAGTCCCTGGGGGTATAGGAGGTCTTAGGAATTG | |
| | | TTTTAACTGCTATCC | | AGGACGTTCAGCTCAA | |
| 342958 | SCAFFOLD | GGCCTAGACTTGTAAGAGAAAACTGGGTGGTCAGCACCT | C/T | GCCACCGGCAAATCTCAGAAGAAACTCCTGTCTGCACAG | 131 |
| | 221402_ | GTTTCACTGTTGCTTCTGCCTTCCCTAGATATGTGCAGG | | CCCGCGGTCAACAAAGACCTTGGCACAGCTGCAAAGTCC | |
| | 10840 | TGAAATTTGTCTGTATTCGGACCCAGTCAAACAGGAAGA | | CCAGTTGAACCATTGTTTTGCTTAGACGAGGTCCAGATA | |
| | | GAGCAGCAGAAGCAGACATGTGCCCAGCATACTTGCTCC | | CCCGCCAAGCCAGAGCAGGAGGACATCGCTCCTTCTGAC | |
| | | TGCGGTACAATGAGAAACTGGATAGACTGTTCATCAGTG | | CTGGCCAAGATAGCCAAAGTGATGAAGAACTTTCTTAAG | |
| | | AACTCAACACACAGCATATACACACTGACCCCAAAACCA | | GTAGATGTGGGTTCCATGGCCTCCTTCAGTGTGGGCAGC | |
| | | CTGGTCCTAGAGGAGA | | AGCCAAGACCTGGACC | |
| 348684 | SCAFFOLD | TTAAAATAAGTAGTCATTTATACAAAATACTCATTTTGT | T/A | AGATGAACCCCATTGCCCAAATTCATGAAAAGAAAAAGT | 132 |
| | 35063_ | TAATTTTGTCCCTGATAGTATCCACAGAAAAGTATGTTA | | TAGACATGTCATGAATGCATAAAACATATGTAGATACAG | |
| | 18919 | TTGGTATAATTAAAAATGAAATAGCACAGCTATACTAAA | | GTCTACTTTACCATTTACTACCATAAAGTATATACAAAT | |
| | | TAATTACACACATACACATAGCTACCCTTGAATAACATG | | CTATTGTAAGAAGTTAGAATTTATTAAAGCTTATGCACA | |
| | | AGTTTGAATTGCATGGGTCCACTAATATGCAGATTTTTT | | CACAGAGACTTTGGGAAATCCTACATACATGGCATCATT | |
| | | TCAATAAATGTATTGGGACATTTTTTAAGGATCTTCTAC | | CCCTGTTAACAGAAATGTAAATAAATGTAAAGATGCAGT | |
| | | ATCTGAAAAAACTTAA | | TTTAAATTATAACTGC | |
| 349112 | SCAFFOLD | CACTCTCTGTCTCAGGTAACCACAAATCTGACCTGTTTT | C/T | AACCGTGTACCTGTTAATCTTCCTCACCTGTTTCTCTCA | 133 |
| | 171377_ | TTTTCCATGAGTTTGTTTTTGAAGTTTAATTTGACCCAC | | TCCCCCAGCCAGCAACCACTTGTTAGTTTTCTTCATCTG | |
| | 3418 | ACAAAACTATATTTAGTTCTGGGTGCATAGCATTGTGAT | | TCATTCTTTTTTATGTTCATTTGTTCTGTTTTTTAGATA | |
| | | TTTATATTTTTATACATTTCAAAATGATCACAATAAGTC | | ACACACGTAAATGAAATAGTATGGTATTTGTCTTTGATT | |
| | | TAGCTACCACCTGTCACCATACAAAGGTATTGCATGATT | | TATTTCACTTAGGTCCTTCCATATTGTTGTTGCACATGG | |
| | | ATTGACTATATTCAACATGCTATATATTTCATACCTGTG | | CAAAGTTTTTTCTTTTTTTTATGGCTGAGTAATACACCT | |
| | | ACTGATTCTGTTTTTA | | GTGTGTGTGTGTACACA | |
| 346975 | SCAFFOLD | AGACCATAAAAACTCAAGAAATGCTTGAATGTTTGTACT | G/A | CCCCACATTTAAACACCGAAATGTATATAGAATGAGATT | 134 |
| | 721_ | TTGTTAGTGCTTTAAGGTAATTTAAAGCAGAGAGATTTT | | ACTGTCTATAGAATGGAAAAGCACATGGCTCACCTCAGA | |
| | 3754 | TGTATCTGGAGCTTTGTGTTCTCAACTCATGATTTAAAA | | CTGTGGTGACTCCCCCTATGTGGCCCAGTCAAGGCGAGC | |
| | | GGTTCTCAAAGGCAGAAATTTTAAAAAAATAATCATCTT | | ACTCCCCATGTATTCCAGTGCCATCTGCTGAAGCCCTCC | |
| | | GAAGCCAAAATGTCAGTTATAACACACATGAACATCAGT | | CTCTCCCCAGCACAGCTGAGCAAGTGAACCAAAAGAAGT | |
| | | GGAATCAGCAATAGACCGCCTATGCTGCCCCACATTTCT | | GGCTACTTTCACTACCTTCTGTCTGGGTGGTGAACAGAG | |
| | | GAACTGAACTGATGCT | | GTGGCAGAGAGACTTA | |
| 345781 | SCAFFOLD | ACTGCTGGGTCATTTTTGTTATGGTAATGAATGCATTTA | G/A | TTGAAGAGTGTATCCAGCTCTGAAAACAAGATGCAAGGA | 135 |
| | 255186_ | GTCCTCAAATTGAAAAAAAAATGTATACTGCTTCTTTTT | | AATGCAGTCCTCATTTTCTCCAGCAGTTAGTTGCTCAAA | |
| | 3403 | ATAATAAGATTTATTTTTATTATGTAGTCCTTTGTTAAT | | GGACTATAGCCATTTCAAGAGGTGCTAGACGTGCATGCA | |
| | | CTTAACTGGGAGATGGTTTTCTTCAAATATTATTGGATC | | TAAGGCTTGATCATGCTAACAATGAAGAAGTATAATGAG | |
| | | CACATTTAGAAGGGCCTGCTTTACAAAGTACGGGCTGAT | | CATTTATGGTTTGAAGAGTTTCAAAAGGATGCAAATCAG | |
| | | AATGTTTTCCAATCAACAAAGCCCTTGAAACTGTTCAGC | | TATCCTATCTACAAGGATAAAAATATCCCATGAAGTTAA | |
| | | TTGTTAGACATCTGCT | | AGAGCAGTTTGTAGAT | |
| 347075 | IBISS4 | GGCTTTCTGAGCAAGAACCTCCTGACAGGACTGAGTAGG | G/T | TTGTTTACAGTACTGGAGAGAAATCCAAGATTGAGGATG | 136 |
| | snp589 | GGTCTAGATGG | | AGTCCAAGTCA | |
| 349265 | SCAFFOLD | CCTTTCATCTTTCTTAAAGTGTCAAGAGCTTTCAGATAC | C/T | GTGTTGACACATGCTGAGCTTGTCATAGTCAGCAAAGAC | 137 |
| | 221810_ | CTCACTGTTCTGAATACTGCTCTGTCAATATTTATTTTA | | ACAAAAATTTTCTATTCAAAGGTGCTATCATATCATTTG | |
| | 557 | TAATATTTATAGTGTATAGTGTTAGAGAAATTATTTCAA | | ATTGTTTTATCCTAACTAGTAAAGGTGGAAACTGGCATT | |
| | | GTGTGAACTGCATAGTAGTTCCTGACGTGAGATTTTGAA | | TGTTCCTTTTAAATTGCATAATATTGATTTAGTTCTCTT | |
| | | AAACACAATGGATAATTTTATTATCTGGACATGAGTCTT | | GTTTTTAAAACAATGATTTGAAATGTTATAGACACTGAA | |
| | | TGATAAACATAATGTATTACCATGTCATGCTAATATTTT | | TCTGAGGGGAAAAAAGAACAATTTTTATATTCAATTCAG | |
| | | CAGAGGTATGATCAG | | TTTAGTTCAGTCGCT | |
| 350926 | SCAFFOLD | CTGGAGTCTTAGGAAATGCAAGTAAATAAATTACTGCCT | A/T | GGAGACTATACACCAGGAGAATGCATCTGACACTCACTG | 138 |
| | 240809_ | ATATTTCTGTCACAAATGGCAGCCAAGCTCTTTATCTCT | | ATGTTTGATCCTCTCTAAGTTAAAATCCTGGAAACAATA | |
| | 7016 | GAATTTGCTCCCTTCTCTTTGTAGGTCAAAAGGAGTCCC | | TTCCAAAAGAGATCTTTGCTATGAGTAATATCATCATTA | |
| | | CAGAACACTAACAGCAATCTTGTTACTACTTCATGAATA | | ACAGTGAGGTACATGGAGCTTATTTTGAAGCTATAAAAG | |
| | | CAGATGATGTTCATTTCCTGAGATTGCCAATCTTTACTC | | AGGATGACAAAAAGCATATATATATGGAAACTTGGAGCC | |
| | | CAAAACCCTAGATCCTGACAAAATCAATTACACTTTCAA | | AGAGAAAATCAGATGGTTTTTAACAAGCTATAGCAGAAG | |
| | | AATGTATTACTCAATC | | AAAGACCACATTTTTG | |
| 343949 | SCAFFOLD | CTTTATCCCAGTGGTCAAAACCACTGCATGACCTTTATC | C/T | GATGGCACCAGACACACGGGGTCAAGGGCAGGTGGACAG | 139 |
| | 155700_ | CTTCACTGCTCTAGATGCCTTTAGCACTGTTGAGCCCTC | | AGAAGGGGCTCAGAAAAGCTGGTGATGCTCTTATTCCTG | |
| | 12715 | CTGATGAACACTGACGACTCCCTCATTCTCCATGAAGCA | | ACTTGAAAACCCTCAGTGCTCTGGATCTGGAGGAGTCAA | |
| | | AGATGTCCTGGTTCTCTTGGTGACTCTGTGAGACTGCTC | | GTGGCTGAGGGATCTTTTTTAGCACTTAGTTTTTTCAAG | |
| | | ACTTGCAGTCTCCTCCACAGTCAGCTGTCACTAGCCCAT | | TCTCACCCAACTGTGGGGTGAAGGTGGGAAGGAGTCGGC | |
| | | TACCTGGACTAACACCATATGAAGATACAGAACATCTGG | | CACGTGTGTAACTTGTGCAAGAGGGAGGCCAAACGCAGC | |
| | | CTGTTTCTACTTCCTG | | ACTCAATTGTT | |
| 345458 | SCAFFOLD | TATTAGAATAATTATTTATTGGACACATTTTGGGCTTCC | A/G | GACGTATTTTGGGGGCAGAATCACAATAAATGTCTAGTT | 140 |
| | 145703_ | CAGGTGGCAACAGTGGTAAAGAACCCTCCTGCCAAAGCA | | CAACTCTCAGGCAAGAATAATGTACATCCAGTTTCAAAC | |
| | 5662 | GGAGACAAAAAAGAAGTGGGTTTGATCCCTGGGTCGGGA | | ATTACATGAAAGAGACTTCACAGCTTTCCCCTAAATGCA | |
| | | AGATTACCGGGAGGAGGACATGGCAACCCACTCCAGTAT | | TTTGAAGATAATTTTCTTGTTGTTTAAATTCATGAACTG | |
| | | TCTTGCCTAAAGAATCACATGGATAGAGGAGCCTGGCGG | | TAACAGTCACCTATTGAAGAAGAATGAACTAGATCAACT | |
| | | GCTAAAGTCCACAGGGTTGCAGAGTCAGACATGACTGAA | | GTATAGATGCGTATCATTCAGATGTTCAGAAAAATATAT | |
| | | GTGACTTAGAATGCAC | | TTGTACTAGAGTTT | |
| 345916 | SCAFFOLD | AGGAGTGGGGATACTCTGAGAGGAGGGTCAGCAAAACAG | C/A | GGAGCCCTGAGAATCCTGTCCATACATTTAACATCCAGC | 141 |
| | 305144_ | ACCCACCCATGATGGACACAGGGCACCCGAGGTCCTCGG | | GGCTCTCCTAAGATGCTCAGTAGGAATTGATCTTGGACT | |
| | 27244 | GACCAGGGGATGTGAGGGGGAGGTCAGGGGTCCACCGGA | | CTTGGCTCCTTAGAGCACCATCTGTCCTTTAAATCTTGT | |
| | | GGGGCACTAGCGACCGTGCAGGCCTCCCTGGAACAGTGA | | GACCCGATCTCCCATTTTTAAAGGACTGTCACAGATCTT | |
| | | GTCAGGGCATCTGTTAGGTCACAGATGACCTAGTGAGGC | | TTGCACGTTTTTGTTGGGCCTTGCTTTGTCCCCTCGTCC | |
| | | TACAGAGTCTGAGGATTCCTGGGTTACTTCCTCTCTATC | | AGAAACACAACAGGCTTCTGTTGTAAGGGCTCCACTCTT | |
| | | AAGGACTTGTTTACCG | | CTGCCCACCATCTAC | |
| 346166 | IBISS4 | GGAAGCATTCCTGGCAAAAATGCAGCTGAGTATGAGGTG | T/C | GAGCCTGGGCTGCTTTTTGAGA | 142 |
| | snp588 | ATCATTGTGAT | | | |
| 346330 | SCAFFOLD | AGACAAATGCAGGGTCTCGTGTCAAAAGAGCTGTCTGGA | G/A | CATTTCTGGGCAACTGATTTTCAGCATAGGTGCTGAGAC | 143 |
| | 145105_ | GCCCAAGACATCGCACACATCTGCCTTAAAGGCTTAGAG | | ATTTCAGTAGGGGAAAGAACAGTCTTTTTCAACAAATGG | |
| | 9149 | TGGGTGCAGAAGACACATTAAGCTCCCCTGTGTGTGGGC | | TGCTGGGATAAGTGAATAGTTCCACGCAAAAGAATGAAG | |
| | | TCATGGAAACAAGGATTTCAGATCTCAAGTTTTCATCTG | | TTGGACCCCTATCTCACACCACATAAAAAAAAAAATTAA | |
| | | ACTGCCTTCTAGGTGAGCTCTGCTCTTATTATACCATCA | | CCCCAGGGGATTTCCCTGTTGGTCCAGTGGTAAAGAATC | |
| | | TGACCCCAAACAACATTTTAAAGCATTCTAGTCAAGAAG | | CGCCTTGCAATGCAGGGAACATGGGTTCAACCCTTGGCT | |
| | | AGGTGACCAACCCAC | | GGGGAACTAAGATCCCA | |
| 353072 | SCAFFOLD | TATTGCCTTGGAGAAGCACCCAGTGGCAATTACGCTTTG | G/A | TCATACAATATTGATCTTTTTATTGTAACTATAATTTTC | 144 |
| | 131294_ | AGATGAGATTTACGGTTATAACCCATAGCAGATCTAACC | | TGAGGGCTGTGAGTAAATAGATTTTTTTTTTGTCACAAT | |
| | 7825 | TTTGGAAGAGACAGGGAATGACAGCTGGGACAGACAGCT | | TATAGTAATCCACTTGAAGCTTACTGTTGCCGTTCAATG | |
| | | TGGGTACGGTCAGAGCAGTGGTCGGTGTTCTCGCCACGA | | ATAGTGGCAAGCGTTTATTTTTTTAATAATAATTCTCAC | |
| | | CTCCAGCATTATAGCCAACACCGGCTGGATGGACTTCCA | | ATGGTTTCCCTTCCAGATTTTACACATAGTAAATGAAAA | |
| | | GTGTGAGTGTCACGAAGCCAGAATTAAATGGCATGTTAT | | AGTAAGCCCACTGAATTGCCTGTGTCTTTTGCTGGCATT | |
| | | ACTTATGTCCCAAGC | | TAGGATAAAGAGAAAT | |
| 350500 | SCAFFOLD | TTTCGCAAAGAGAGGGCTGTGTTGGCAGATGCAGAGACC | A/G | TCTCCCACCACTGAACACTGCTCCACCTGGGAGCAGCCA | 145 |
| | 70668_ | AGCTCCCCAGATGGAATAATCTTTTTCTTATTAATTTGC | | TTGCTCTGGTGTTTTTTAGGAAACGACAGTCTAGATGGT | |
| | 8488 | TGTATGTTGCTTCTCATTTCTCACAGTCACTCCAAATGT | | TTGAGTGATGTTTTCAGGGCCCTGGAAAGAGGTCGAGGT | |
| | | GTTACAAACGACTACATGCTCTGGGTCCTGAATCCACTA | | GTGGCTGGGATTGAAACTCACAGTTCCAACTTCTTGGGC | |
| | | TTGCTATAGCTTTGGGAAAAAAAAATATTGACTGTCAAC | | TGTGGAGTCTTCCTATTGTCTAGCATATGAAGCAGGCTG | |
| | | TGCAGTGAGCTTTCCCAGCACTTTTTTCGTGCTCGATTT | | TTTACTCCCTTCAGGCAAGGGATTCATGAGCATTGCCTG | |
| | | TTAAATTAACATTACC | | GAGAGCTTGTTAAAAA | |
| 344038 | SCAFFOLD | AGCAACTCAAACTAAAATAGGTCAAAAGCAGGAAAAGCT | G/A | GGCAACACCCCAATGCCCTGGCTTCTTCTGCATCCCAGC | 146 |
| | 200024_ | GAAGGCTTTCCCTAAATCACACAGAACTCAAAGCACCTA | | TGGTTTCCCTTTCTGACTTCCCCTTGGTATGTGTGTCCC | |
| | 14061 | AAGTAATCTATGTGAAGATAAAAAGATGAGTGGCAGATG | | TTTCTCTGACTGTTGTCAGAGGGATGGGTATTTTCCTGT | |
| | | GCATCACTCTGCCAGAGATGGGAAGTTAAAATAAATAGC | | GTTCATTATTTCCAGAGCAGACAGATTTGCATTAGCCTC | |
| | | AACCAAATACCAAAAACATGAATAGACAGAAAAACTCCC | | ATGTACTTCTAAGCATTGGTGTCAGCAGCTTCTTGTGCT | |
| | | ATCCAGATGTCGGTGTATTTGAAGCACAGAGAAAGTGGA | | CCAGATCACATCCTCACGACCCGCATTTTTTACAGCAAC | |
| | | ATTTGGTCCTAGGCAA | | CAGCTTGGTGAAGGTG | |
| 349617 | SCAFFOLD | AAGATATCTCACAACTGATGATGCACATCTTGTGATTGA | C/G | AAACTGAAATGCTCATCAGGCCTCTCCCTGGTCCCTTCA | 147 |
| | 50212_ | CAGACTGTGGGTGTTGGTCCTGCCCTCTGTAGTGTTACC | | GTTTGGTTTGCAAGGCCCTCCGTGATAGGATGCCAGCTT | |
| | 11110 | TTGGAAAAGTATGTGTGACAGTCACCAAGCACCACCAGC | | AGTATCTGGCCTCATCTCCCTCCCCTGACTTCCATTCCA | |
| | | ATCCACGGTCAGGCACCAGCTGCCCACTCAGTACCTCTC | | TATCTTTGATGGGCTATTCCTTCTCATGCCTGTAAGCTG | |
| | | AAATGGAAGCAGTGGAGGAATGGGTTCTCCAAGGAGTTG | | CCACATGTGTCAATCCTTACATAGGGATTTCTACATTTC | |
| | | TCCACCCAGTCTCTCTTTCTCCAATATTGCACCCATCCA | | TACATAGCACATTTTCTACTTTTTTTTTTTCACCTGAAA | |
| | | ATCTATCCAATTCTTT | | ACTCTTATCTATCCCT | |
| 351664 | SCAFFOLD | GAATGCTTCCTTTTAGCCTTTGCGATAATCCTACTTAAG | C/T | AACAGTTGTTTTAAGAACAAAGCTAGTTACTTGAGCTAT | 148 |
| | 266321_ | TGCATACTGTTATCAGTCCCATTGTATAGCTGAAGGAAC | | CTGTATGATTATTCCATGACTGAGGACATAAATTCACAT | |
| | 6651 | TGATTCTCAGCAAGGCTCTTAAAGTAGCTTGCATAAGGT | | TTTAGAAAGTGTGGCTGCATATATTCTTTATTAATCAAC | |
| | | TTCACATTTCAGAAGTTATGGAGCAGTTCATGAGTCATC | | TTGCAGAAGAATTACCAACCTGAAAAAGTTAATTGTAGA | |
| | | TGATGGAGCCAGTGCCTTTGATCTCACATTCTCAGCTCA | | GGTTAATATCTTCCTCTGAAGTTTGCAATAAGGAGTTCA | |
| | | CTTATCTTAGAAGAATTAAGGTGGATTTAGACTCAACAC | | TGATCTGAGCCACAGTCAGTTCCTGGTCTTGTTTTGCTG | |
| | | TTGTTTCTTTAAGATG | | ACTGTATAGAGCTTC | |
| 346539 | SCAFFOLD | CGATTCCCAATTTCTAGAGTCTGGCTTATCGGTCTATAA | C/G | ATTCTATCCATGGAGTTTTCCTGCTACTTAGTGAATCTT | 149 |
| | 210756_ | AACAGGGAGTTTAGATTCTAACATGAGGGCCATTTCCTT | | CCTTGTTACCATGTTATTGATTCCCCCCACCCCCAGGAG | |
| | 13983 | CGTAGGTGGGACTGGTCAGAGGTTAGCATGAGAAGACTG | | ATAGCTGTACTATATTGCATAATAATAAGATGACTATTT | |
| | | CTGAGACCTGCCCGCCAGGACATCCAGGGCACCTCACTT | | AGTGTTAGCTCAGGGCCTCTAATGAAGCAGGCGTCAAGA | |
| | | GAGAGCCCTGCCTTGGGAAGTGCTTCTCCATCTGTATTA | | TGGAACTAGGCATGGAGGGATTTATTGGTTGACCTTCCT | |
| | | GCATCATAGCATCTTTCCATCTGCATTTATGATGTGATT | | GTGGAGGACACAGGGCAGTGGGGGCAGGGGTAAGAGGGA | |
| | | CACTTCTGAGCAGCTC | | AGGCCATTCAGACATT | |
| 351855 | SCAFFOLD | TGGAGGGGAGTGTCCATCAGGTTTGGCCTGCTCAAAAAA | T/A | ACATTTATATTTTGTCCCAGTATAAGCTTATCTGCTTCT | 150 |
| | 55579_ | ATTTCATCTATGACCTCCTTGCAGTTATGATCGAGGGTT | | CTGACCAGGACCACAGTGGCAGCCAATGCCCAGAGACAT | |
| | 11155 | CCCACCTCTGTAGGTAAGTAGGTAAGTAGGTGGTGGGAT | | GGCAGCCATCCCGTGGCCACACAGTCCAGTTGTTGGACA | |
| | | TCAGTGTCCGCACAGTCTCGAGGTGGACCTGTGGGTTTT | | GGCATATGACCAGGCGCTGCTATGGTCCAACTGTTTGCA | |
| | | CACAAAGCAGTCCCTGACAGTGAGTCATCCTGGTATTGG | | TGAGGACCCCCAGTGCAGTGTGATTTTTCTCATGCACAA | |
| | | TCAGTTACTTGTGCCCTTGGCTGTTCATCAGCACAGTAA | | ATAAGTTAAAGTCCCATGTCACATCTGGCAGCCCTAATG | |
| | | CAGCATGGGGAACTTT | | CTGGAGCGTTGGTCA | |
| 353087 | SCAFFOLD | CTGCTCAGAGATTTGTGTAAAATGACAGGTGGTTTAGTA | A/T | ACAATTAAATGATAAAACTACATTTCTACATGGAGCTAT | 151 |
| | 145449_ | AGAAATTCAGCAGTATCAGGAAATTCTATAGGTGATCGG | | CATCAAGACATTCTACGAATTCCGGCAGAATATAGACTA | |
| | 8789 | TTAGCTGGAGAGCCAGCAGACACTGGTCCATGACTGGGT | | TATTTGTTATCATCAATGACCGTGGAATAAGCACCCTGT | |
| | | GAATTCATGCTTGGTAAACCACCTTAAAAATTGAAAGAG | | GGCAGGCTGACACATAACAGTAAATACTAAGAGTACGAG | |
| | | AAGAAAGAGATATTATGTATGCATGTATCTGTTTATTGT | | GAAAACGTACATAATAATGATAATAACGTGTGTACACGA | |
| | | CTATGTTTGGGTACTTATAAAGAGCATTCCCCTTCTTTA | | TAAAATGTGTGGTACAGATTTTCCTAATCCATATTGATC | |
| | | AAGAAAGCAAAGCATT | | TTCATCTGTCACATTTTG | |
| 350312 | SCAFFOLD | GTGGACACAGTATGTAATGTCTTTTTTCTTTTTATTTTT | T/C | CCAATGACATTCTTTTGTGTTACGTGACCTGGTAAAACC | 152 |
| | 295543_ | TCACTGCTCTTCAAATGCCCTTCCCTTCAATCTGTTATA | | ATGATCAAATTCACCTCCTGGCCCACCTGGCATTTCCAG | |
| | 3871 | GGTCTTCCTCAGAAGAGTCCCTTGTGCCATAAATTTTTT | | GGAGGGACTAGGATTGAATCAAGGAACAAGTTGCATTAA | |
| | | TACTTGGCTTCTTGTTAAGTCAGGACTACTCTTCACGCT | | GTCAGAAGTGTGTTCCCTTTCCATCTGCTTTGAGACAGA | |
| | | GTGCACCCATGAAATCATTGTCCTGCGGTAGACCTTCAA | | ATCCTTCAGTGATACAGAATCCTTCAATGATGTAGAATC | |
| | | AGAAGCCACATGGTCTAGCCCGAGGATCTGCCCTTGTTA | | CAAGCCTTAGAAGACGCGAGGTGGATAAGAATCGACTCG | |
| | | ATTGGGAGTTAGTCCG | | GGCCACAGCCAAGAAG | |
| 352162 | SCAFFOLD | TTTAGGTCATACCTGAATGGTCTAGTGGTTTCCCTCACT | T/C | TGAAATCCATAACCAGAGTGAAGACTTATCTGATAAATA | 153 |
| | 205195_ | TTCTTTCAATTTAAGTCTGAATTTGGCAATAAGGAGTTC | | TGCAAGACCCAGCTCAAGAGCAAACAGTGGGGCAAATTC | |
| | 11563 | ATGATCTGAGCCACAGTCAGCTCCCAGTTTTGTTTTTGC | | TGGCATATCATAGCCACAATAATCCTAAGAAGATCCTAG | |
| | | TGACTGTATAGAGCTTCTCCATCTTTGGCTGCAAAGAAT | | ATACACTATGGAACATAATGAACCAGCTGACCAAATACT | |
| | | ATAATCAATCTGATTTCAGTGTTGACCATCTGGTGATGT | | ACGGGTGTCAATTTGGCACTAATAATCTAAATAAATAAA | |
| | | CCATGTGTAGAGTCTTCTCTTGTTCACTATGCATAATAA | | AGATTCACACATTAGAATTTGGTATATGCTGAGAAGTAT | |
| | | ATCTGTTGTCTGAATAT | | TTATGTTGAAGCTGAA | |
| 345166 | SCAFFOLD | AGTCCAATACATATTGTTGTAAATGGCAAAATTTAATTA | T/C | GACTGAGCAACTAAGCCCATGATGAGTAGTATTCCATTG | 154 |
| | 60902_ | TTCTTTATTATGATTAGTGGGCTTCCCTGGTGGCTCAGA | | TGTATGTACACTACATCTTCTTTATCCATTCATCTGTTG | |
| | 6944 | AGGTAAAAGAATCTGCCTGCAGTGTGGGAGACCTGGGTT | | GTAGACACTTCCATATCTTGCACTTGTAAATAATGCTGC | |
| | | TGATCCCTGGGTTGGGAAGATCCCCTGGAGAAGGGCATG | | TATGAACATGGGTGGGCATGTACCTTTTTCAAATCAGTG | |
| | | GCAACCCACTCCAGTATTGTTGCCTGGAGAATCCCCATG | | TTTTTCATTGGATATGTGCCCAGGAGTGGAATTGCTGGG | |
| | | GACAGAGGAGCCTGGCAGGCTGCAGTCTATGGGGTCACA | | TCATATGATAATTCTGTTTTTAGTTTTTTATGAAACCTC | |
| | | AAGAGTCAGTCAGACA | | TATACTGTTTTCCAGAG | |
| 347646 | SCAFFOLD | TTCAGGATGGCCTGAGAAATGCTCCCACTGTTTCCATTA | G/C | AATTCACCATTTTCCCCAGATGGCCCACTACTTCCCTCC | 155 |
| | 290023_ | GAAGGTAAGATGATCAAACCGTTAGAGGGTGTGCTGGTT | | TCCAAATAGGAAGACCCAACAGGACCAAACAGGGCTTGC | |
| | 4337 | TTCTGGGCTTCATTTGCCACGGGTAGCACTAAAACCCCA | | AGTGGCCCCTGTTTCCAGAGCCAGGATTCCCAAGCCGGT | |
| | | GGTCATCACTGCTGCCTCTCTTCCAGGCTGCTCGGTGAT | | GTTGTACCTGATTGAGCAGGTGCCAGGTCTGATAGTCCT | |
| | | TTCAATGAGTAGATCAGGATTCAGGGTGCACAGCAGTTC | | CCTTGCTACAGTGTCGGCTGAAGATTGATTTGTAGTCCA | |
| | | CAAAATATCATATGCCCTTGCTCAACCTGGTGTGTGAAT | | CCTTCACCAACTGCCATTCCGAGCGGAGGCTGAAGTGGC | |
| | | TCCAATCCCACCCCCA | | CAAAAACTCTAAGTGT | |
| 354124 | SCAFFOLD | CCTGCAATGCAGGAGACCTGGGTTCAATCCCTGGATTGG | T/A | ACATAAAATTATTTTTGTCAAATTGCTTTAAAATATTGG | 156 |
| | 135419_ | AAAGATGCTCTGGAGAAGGAAATGGCAACCCACTCCTGT | | CAGGCTTCCTTTCCATTTCCATATTCACCTTTTGGCAAA | |
| | 14068 | ATTCTTGTCTGGGAAATCCCATGGATAGAGGAGCCTGGC | | CCAGTAACACATGGCTCACAGACTGGCCTGCTCTGTTAG | |
| | | AGACTAGAGTCCACAGGGTTGCACACAGTCGGACACGAC | | TCACATTTGAAAGAGTTGTAACATGGATGTGACTTTAGT | |
| | | TTAGCAACTGAACAATAACAACAAAAAATTACTAGAAAA | | ACTTTATACCAGGAAGAAATGGACTTAAATGGCCCTGAT | |
| | | AAAATTTTTAAAACATACAAATTCAAGCCTCAATTTTTA | | GAGGACCCCTGATAGAATATACTTTGATGAGGCTAAGAA | |
| | | TTATTAGAGTCAACA | | AAAAAAGAGAGAGAG | |
| 345428 | SCAFFOLD | CTTCTCATACCTATTTTTGTTTCTGGTGTTGATAGAATT | T/C | TTTTCTTCCAGGGAGCAAGCTCCTTTTAATTTCATGGCT | 157 |
| | 135007_ | GACTGGTTTGATCTCCTTGCAGTCCAAGGGACTCTCAAG | | GCA GTAACTGTCCACAGTGATTTTGGAGCCCAAGAAAA | |
| | 48819 | AGTCTACTCCAACACCACAGTTTGAAGACATCTATTCTT | | TAAAATCTGTCACTCCATTCATAGGTAGCTCCAATAGGA | |
| | | CGACCCTCAGCCTTTTTTTGTTGTCCAGCTCTCACATTT | | GAATAATTCCTTGATAAATTTACTTTTGTCTACTATTAA | |
| | | GTACATGACTACTGGAAAAACCATAGCTTTGACTACATG | | CTACATATAAGGAAAAGTTAATGGATTTATAAGACAGCT | |
| | | GACTTTGGTAGGCAAAGCAATGTCTCTGCTTTTTAGCTG | | ATTTAGTAATCAGATTTTTAAATGGTATGCTATTACATT | |
| | | TCTAGGTTTCTCATTG | | TTGAAAATATAGAAA | |
| 346083 | SCAFFOLD | AAAGATTGGTCCTCAACCCTGAGCTTCACCCACAAGAAA | T/G | GTTGAGCAGAGTTCCAACAAATGGAACTTATATTCAATA | 158 |
| | 61272_ | TTTCACCCACACTTTAATGAACTAGTCACTCTGCCCCCA | | CAGATGTCCATGTTTATTGTCAAATTGCCAAGAAAATAT | |
| | 3342 | AACACAAAATGGAATTTTATTTTATTTTTTTAACACCAA | | TCCCCATTACCCTCTTCATTAAACCAGATATTATAAATG | |
| | | AAACATTTTGTATTGGGGTATGGCTGATTAACAATGTTG | | AGGAAAGAAGAGTGTTAAGATCAGAACTGTATCGTCCAG | |
| | | TGATAGTTTCAGGCGAACAGCGAAGGAACTCAGCCATAC | | GAAGCACACTTGGTTTGTCTGCTGCCAAGTGTGGATAAT | |
| | | ATATACTTGTATCCATTCTCCCCTAAACCCTCCTCCCAT | | ATGCCTATTTGGTACACACTCAGCATTCATGTTCTGCCA | |
| | | TTAGGCTGGCACATAA | | AGCAGGCGTGAGCTT | |
| 348221 | SCAFFOLD | ATATTAACTGTTTATCTCTGCTTGGTGGGGTTATAGTTT | G/A | CATCTACTTCACAAGGCTGTGTGCATGTATGTGTGTGTG | 159 |
| | 157123_ | ATTTTTATGTAAGCTTTTCTGTATTAAAACTTTATTTTC | | TGTGAGATAATAAATATGGAATCCCTTTGTAACTCCAAA | |
| | 901 | AATGGGAATGTAATACTTTATATTAGACATTTAGGTTTT | | GCTTGCTAAAGGTGTCAGCTGGTATTATAATGATGATAA | |
| | | TTAATTCCTAATTGAATATGTTTGATAGCTTCTATTTTG | | AGTCCTGTGACCTAAAAGAGGTGAGAGAACCCTTATGAT | |
| | | GCACATGTCAGCCCTATGGAGCGAGGGAGCATCAACCTA | | AACAGGTAACTTTGTGCTGAGAGAGGAGGGAGATCTAAG | |
| | | GAATTCAAACAAGTTTTACCAGTCATTTCACCCGATCTG | | TGTGAGGACAAAAGTGAAAATATGTTTTTTTGCCTTAAA | |
| | | TAAAATGGGTACAATT | | CAATCACATGCCTGAC | |
| 346860 | SCAFFOLD | TTTTCACTGCAACATCTTTGCAACTTATTAATAGCCAGT | T/C | AGGACACTCTTAGCAACTGCCAGTTTAGATCCTCTAGAA | 160 |
| | 35037_ | CAATTTCATTGTCCTTCAATGTGTTTCATCCTTCATATT | | AGGTGATACTGAAATTAAATTTGAAGTGCAAGAGGGTGG | |
| | 3980 | TATCAAAAGTATGCATCATTATCCTACCCAGAGTAATTC | | TGGTGGTTGTTGTTGGGTTTTGGAGGAAGAAAGTGGTAA | |
| | | CTTCCAGTGGAATATCCTTCCTACTCTCCACCAGTGAGT | | CTCCTGTAAAATATAAAGGGAGGACATCAAAATGGTTCT | |
| | | TTTAGCACCACTTTTTACAGGAACTGTGTCTCACATACT | | GAGAAAAGCTTAAGACCATAATACTGATCTGTGCATGTG | |
| | | ACTTGGGATGGCAAGCGATCCACTCCTCAGATTCCAATC | | TGTGTGCTAAGTCACTTCTGTTGTGTCTGACTCTGTGTG | |
| | | CCACTTACCAGGTATT | | AGCCCCATGGACTATA | |
| 350535 | SCAFFOLD | CTTAACTAAATCTACCATTTTTAGTAGGTATGTCTTATT | A/G | CTTCTGTCTGGATTAGTTTTTGCAGCATACCAAGTTAGC | 161 |
| | 81100_ | TTTCCAACCTTGCAACAGTTTAGCCATACTTTTTCAGCC | | CTAAGATTTAGTAGCCTAAAATGACATCGTTCTGTGAGT | |
| | 3471 | ATTACATAATATGGAATGCCTTTTATTCAGGGTCCATTT | | TGACTACTGTTGATTGAGTGGTTCAAACTTCGAGTTTCT | |
| | | ACAAAATCTCACTGTCTTCCATCCTTTGCTAAGAGTTTC | | TATGAGGTTGTAGTAAGACAGCAGCTTAACCTACAGTCT | |
| | | CTTGAGATACTCAAAAGCTCTACTCTCTCCCTTATCCTA | | TCTGAAGTCTGACTGGGCTCAGCATCGAAGATTGCTCAC | |
| | | AACAATGTCAAACATTATAGTTTTTGTTATAGCAGCATT | | TCATATAACTTGTGGTGCAGAAATGCTGGCTATAAGCTG | |
| | | TAAATCTTGGGTGTTC | | GAAGCTGACTATAACT | |
| 348791 | SCAFFOLD | TATCTTACAGTTCTGGAGGTCAGAAGTCTAAAATAGGTC | G/C | TTTTGCCATGTGTGGCACCAGAATAAGAGGTTCTGGGCA | 162 |
| | 66709_ | AGCAGGATTGTATTCTTTCAGAGGCTCTAGACAGTAATC | | TTAGGACATAAACATATTCAGGTGCTTCTGGTTCACTTA | |
| | 2463 | CATGTCCTTGCCTTTTCAAACTTCAGAAACCATCTGCAC | | CTGATGATGGAGAATATGATGAATGAATAAGCAGGAAAT | |
| | | TCCTTGGCTCTGACCGCTTCTCCTCCGTCTTCAAAGCAA | | TCTAGAGATACAGTTAGAAAGCTATGGGCAATTGTTCAG | |
| | | CTTCAAATCTCTTTCTCTCTCTCTTTGACTCTGATCCTA | | GGATACAATAATCAGTGACTGGTGTATCAGACAGAAAAA | |
| | | GATATTCTGGGAAAATCTTCCTATCTCAAGGTTCTTAGT | | CAAGTTATCTATTTGCAACTGATGAAACAGTGAGTTAAC | |
| | | CACATACACAATATCC | | CAAGACTAGTTTGGTA | |
| 353906 | SCAFFOLD | TGAAGTTACTTATCCAAAGCTATAGAACACAAAGTCTTG | G/A | AAAATCAACCCCAAAAAGTCTAGGGGCAGGTCAAGAAAT | 163 |
| | 41654_ | CACACATTAGGTCTTAAAATATAAATTACATGTTAGTTG | | GTATTAAAAAAATATGGAAGAGATCTCAGCATGTTATCC | |
| | 3423 | TTATATTAGCAAAAGAGGCAGACCTAAAATATCTGGAGC | | TGAGGGAAGGACAAAATATTCAGTTTCAAGTACCACTAC | |
| | | ATTTTTTTCTCACCAAAATCACTGTTGTTTAATAGGTTT | | TGCACAGATCTGGTGGGACCCAGCTGTTACTAGAACCAT | |
| | | GTTTTTTTTGTTTGTTTGTTTTTCCATTTGTCTGGAAGT | | GAATGCTTCATTTTGCACAGAGAGGTATTTACAGGGAAG | |
| | | TCTTCATTTGTCTGGAAGTGTTCAAAAGAAAGTATAAAA | | TAATTTCCTAAGAGCAAGAGCTCTGGTCAACTCGGATTG | |
| | | GAAAAATGTTCATTAT | | GGTTGAAGGCATGCTGT | |
| 348449 | SCAFFOLD | ACAAAATCAATTACACTTTCAAAATGTATTACTCAATCA | T/C | TTTTAACAAGCTATAGCAGAAGAAAGACCACATTTTTGT | 164 |
| | 240809_ | GGAGACTATACACCAGGAGAATGCATCTGACACTCACTG | | CCCCTTAAACCAAGTTTTAATGAAGAAAAGATTCTGTTA | |
| | 6804 | ATGTTTGATCCTCTCTAAGTTAAAATCCTGGAAACAATA | | TATATGATTTCCAGTGGTTTAATATGGTGGATTGAGAGA | |
| | | TTCCAAAAGAGATCTTTGCTATGAGTAATATCATCATTA | | CTCATTGTCATGCCCTTGAGAAATGAAAATAACTGGGTC | |
| | | ACAGTGAGGTACATGGAGCTTATTTTTGAAGCTATAAAA | | TTAATTTTTTGCTCTAACATTAATTGCCAGACATCAAGC | |
| | | GAGGATGACAAAAAGCATATATATATGGAAACTTGGAGC | | CACTCAAGGATGGGCTATCTCATATAGACTATCTGTTTT | |
| | | CAGAGAAAATCAGATGG | | CTAAAATTTAAATCTG | |
| 349343 | SCAFFOLD | ACTCCAGGGCATCTCCCTAAACCAGGGATCGAACCTGCA | A/T | TCAGTTCAGTTCAGTTCAGTTGCTCAGTCATGTCCGACT | 165 |
| | 249581_ | TCTCTTGTGTCCTATATTGGCAGGTGGATTCTTTACTAC | | CTTTGCGACCCTATGAATCACAGGACACCAGGCCTCCCT | |
| | 1039 | CTGTAGAAAGACTTTATTTTGTAGGGCAGTTTTATATTC | | GTCCATCACCAACTCCCAGAGTTCACTCAAACCCATGTC | |
| | | ACACTGAAATTGAGAGGAAAGTACAGAGAGTTTCCATGT | | CATCGAGTCGGTGATGCCATCCAGCCATCTCATCCGCTG | |
| | | ACTCCTTCTCCCCCAAATATACACATTCTCCCCTACTGT | | TCATCCCCTTCTCCTCCTGCCCCCCCTGCCAATCCCTCC | |
| | | CAACATCCTGTACCACAGTGAAACATTTTTATAATTGGT | | CAGCATCAAGGTCTTTTCCAATGAGTCAACTCTTCGCAC | |
| | | GAGACTACATTATCAG | | AAGGTGGCTGAAGTAT | |
| 346663 | SCAFFOLD | TAATTATTTTTTCTCTTGTTACTCTATTAGAACAGTTTT | G/C | TAAAACATGGTATCCCTTGTGCCACTTACCCTAAAGGTT | 166 |
| | 26010_ | TTATTAAGATTATTTTAGTTTCATTGTTTATTTTTACTA | | TCTAAGCCTCTATTCCTTCTGGAAAGTGGACATGACTCT | |
| | 1655 | ATAACAGTGCCTGATTCAGTCCTCATAAAAGTTGCAATG | | TTATGCTGCTACTGCCCTTACCAGAGTAAATTTCACTTA | |
| | | TGACCTCAACATAGTGGATTTTTTTTGTGTGTGGAGTAT | | ATTCCAATGTTTTTGAATTTTTACTTTCTAGGTTAAAGA | |
| | | TTGTTAAATTTTTTTTACAATATTAAAAAGCAAAGATAA | | CTGAAATGGGTATATCTGATTAGCAAAATCTATAAGTGT | |
| | | TAGGTAGTAAAATGATTTTTGTATATTTTTCTTAGCTTC | | AAGGCAAGTTGGGAGAGCAAGAATCTGTAATTTCCAGCT | |
| | | TTGCCAGTGTCCATCA | | TATTTTTGGAAGGTG | |
| 349798 | SCAFFOLD | CAGAGGTGTAAGCTTTAAGGGTCCTGTTTTCCATAGGCT | T/A | AGAATCTTCATTTTAACAAGTCTGGCACATAAAGTTCCT | 167 |
| | 105315_ | TTTGTAATGCTGAGACAGAGCCTAGTTCCATTTGCCTCT | | CTGACAGAGAGAGCAGCCCCTCCCACTCCACTAAACCCA | |
| | 6308 | TGAAAGCATTTTAAAGGTTAAGAGAAACATTTAGTACTA | | ATTCTGAAAGGTTTCCAGTGAGGTCTTAGAGGTAAAAAC | |
| | | TGGCATTTTTCTCTATTCCAATGGCTCTTAAAGGTTTTG | | CAGTGCCCTCCTCTAATGATCTCCTAGACCTTTTGTTAG | |
| | | ATATATATAAGGATCACTTGGGGAAGTTGTCAAAAATAC | | CACCCATCAGTGGGTAACCCCACCTTCATCTATGCAGTG | |
| | | ATATTCTCTAGGCCTCAGTCTCAGACACTACAGTGTGCA | | TGGCTGCTGTGCAGAAGTCTCTCAAAGGGGCATCAAGCT | |
| | | GAGTCTAAGCAAATTC | | TTACACTCTCTCCTG | |
| 351627 | SCAFFOLD | AATATCTACCTCCAGGCTTTAAAAAAGGAAGAGGAGAGA | G/A | GGATTAAGGGATTAGCTTCAATCCGGTAAAGATTTTCTC | 168 |
| | 255096_ | CCATGCCTAGAAATAAAACTAAATAGCACAGAGTTCCGC | | TTTTATAGAACAACCTGCTAAACATCAGTCCCTACGAGT | |
| | 4614 | CAGCTCTGTCCCTCCTTGTCTTCGTGACCTTGGCCAAAT | | TGCCAGCACACACACATACATACCACAAGTGTGTGCCCT | |
| | | CACATCCTTTCCTTTTGCATTAGAAGGACTGATTAATCC | | AATGTTCCTTCTTGTCTCCCCACGTGGTTTTTTCATCCT | |
| | | CTGTCCCGTGTTCTTTCGTGGCATTTGTGAAGATTAAAT | | TCCTCCTGCAACACTCTTGCTCAACATAAAGATCTTTAT | |
| | | GTGATAAATGGTTGGAAAAGTGCTTGAAGACTGTAAATT | | GCATCCTGACTTTAGGGAAACTGTATTATGTTAGACTTC | |
| | | GATGTACAAATGGAA | | TCATGCCTGAGGGCAA | |
| 352113 | SCAFFOLD | AGACGCAGGTTTGATCCCTGTATCTACAAGATATCCTCA | G/A | GTAGAAATATTCAGTTAATTTTGCTACAGTGAAGTTGGG | 169 |
| | 170528_ | AGAAGAAAATGGCAACCCACTCCAGTATTCTTGCCTGGG | | TTCCTTCATTATTCTGCATTTCAGTTTGTTTTCCTGTTT | |
| | 2229 | GAATTCCACGGACAGAGAAACCTGGTGGGCTACAGTCCA | | CATAACAAGGGCTTTTTTTAGGATTTTTAAGGAATGTTG | |
| | | TGGGGTTGCAAAAAAAGTCAGATACAGCCTAGCGACTAA | | CCTTGAATTTATATCTCTCAAAAGTAACAAAGTTTTATC | |
| | | CAACAAAACAACAATTTCCAAAGCTCAAAGATAAATAGA | | TGAGAAAATATCACTTGTACGTTTTAATGAAAGCAGGAG | |
| | | ATCATTTGAAATAGCATCCATATATACAAGTGGTTTTAT | | CCATTTGGGGAAAAGCTAGAGACTTGGATATACGTGTTT | |
| | | CCTGACCTTTCTGACT | | TGGTACCCAGACTGG | |
| 353576 | SCAFFOLD | AAAACTGTCTTCCACGAAACAGATCCCTGGTGCTAAAAA | C/T | GCCTTCAAATTAGTTTTATTTCATCGAGGGGCAAGAGTA | 170 |
| | 245583_ | GGTTGGGCCTTACAGGATTGAGAAAAGAGTGCTATCTCC | | CTTCAGTGGACCATGTTCCAAGTTAGAAACATGTTTCGT | |
| | 8789 | TAAGAAATTAGCTTGCTGGCACCAGGAGAAAATTGCTCT | | CAATGTGGATTTAGATTGTAACACGTGTGTGTGTGCTGT | |
| | | TGTCAGTCAAGCAGGCATTCCTGTGTCCTCTAAGATGTC | | TAGTGTGCCAGTGTGTCCAAGTGCCCTAATAGTTGTATC | |
| | | TGGTTAAGTGAACGCAGGTGCAGGATGCACACTAAAGAG | | TAGGATCACTCAGGGGAGAAGGCAATGGCACCCCACTCC | |
| | | GGATAGTGGTTCAAACGAGCAGGGAGAGAATTTATGTTT | | AGTACTCTTGCCTGGAAAATCCCACGGATGGAGGAGCCT | |
| | | AAAATTTTTCTAGCCC | | GGTGGGCTACAGTCCA | |
| 348728 | SCAFFOLD | TTGGATCTCCTTGCAGTCCAAGGCACTCTCAAGAGTCTT | G/A | TGTATCTTGAATCCCCTTTTATCGTTTCTGTACACAGCC | 171 |
| | 47314_ | CTCCAACACCACAGTTCAAAAGCATCAATTCTCCGGTGC | | CTGTGGAGGACTTCTCTTCAGCTACTGGAGTTGTTTAGC | |
| | 3413 | TCAGCCTTCTTCACAGTCCAACTCTCACATCCATACATG | | CCAAAGTATGTGAAGGCTTACCTCCTTGCCTTGGGTCAA | |
| | | TTTCACTCTTTTTTATGGCTAATAGCACTGCATAGCACC | | GACAACCCTGATGGATTAAGCACTGTTATAAGCACTTTT | |
| | | ACATCTTCTTTACGCCATCTATCTGTCGATGGTCATTTA | | ATTTGCATAATTTTCTTTAATGCTTTCAACTACTAACTA | |
| | | GGCTCCTTCCGTGTCTTGGCTGTTGTAAACAGTGCTGCT | | AAGCAGGTACTATCATTTTAAGGAAAAATTTTTTTAATT | |
| | | CTGAACTTAGAGGTGC | | CAGAGAAATAAAGTAA | |
| 348913 | SCAFFOLD | AAGGCATATACATCAAACTCACCACACTGGTTGCCTTGG | T/C | AGAGTTGAACTCAGCTGCTAGTTTCACTCTGATATTCTG | 172 |
| | 103011_ | GGGATGGAGCAGAAACATGGGCCCGAGTTTGGGGACAAA | | TTTCATGGGAGCCAAACAAATCTCTTAAGAAATTTTGAT | |
| | 2035 | GAAGACTTCGATTCCACCGTAATCTTTAGTTGTGTTTAA | | TCGTAAAACTTAGAACTGAGATATGCAGTGGAGACCTAA | |
| | | AACCATCCAACAAATAATTACATCATCATTGTTACCTTA | | CTCACATTCAGGGAAGACAAAACATAAAGAACATCAGTA | |
| | | GTGTAGTGGGTACAAAATTACCTGTAAATTATTTTCTGC | | ATTCTGGTCATGGTGCCTGCCACTCATGGCTTTTTAGGT | |
| | | ACTATTCTGTATTTTTCTTTAATTACTGGAGATAATAGG | | TTCTGATTTGGTTTTCTCCTTATGTAATCAAATTTGTAA | |
| | | TAAAAGGAATGAGAA | | TCAAATCATACATGCA | |
| 349578 | SCAFFOLD | GAGACTTGGAAATCTGGCACGAGATCACTCAGGAAAAAG | T/A | CTGTTCCATTTACCAAAAATTAGTGGCAACTCTAAGAAG | 173 |
| | 350635_ | CGTGTGGAGTGAGAAGAGAAGAGCGTCTTGGACAGAACT | | AAGAAGCCTCAGGGAGGCCGTGAGTGAATAAGGCCAGGC | |
| | 386 | CAAAGGAACTCCAAAAGCAAAGGGAGAAGCAGGGTTTCG | | TGCAACGGTTTGAGAGCATGTGAAAAGTGAAGGACAGAT | |
| | | TGACAGAGGCGGGAGGAAGAGGCAGAGCAGGAGGAAAAC | | CTGATGAAAAGGAATTTAAAAAGTTGGAGGTAATTTCCT | |
| | | CACAGGAAGAGGCACAGGAGCCTAGGAACACAACGGAGG | | TCTTTACTGTTCTCTCTTGGCTACTGAGAGTCTACATTT | |
| | | CAGTGTCTGGGTCAAATGCTGCTGCGAGGCCAAACAAGG | | TCCAGTTCCACCAGCAATGTAGGGGGTTCCAGTTTCTCC | |
| | | TGAGGACTGAGAAACG | | ATCTTATTGTCTGTCT | |
| 347472 | SCAFFOLD | TTTGCCTAGAAATGCAGCCCATAGCATTCATGGGTTTAC | A/G | GTTGTGAATTTTATTTGGGTTCTTCAATTCTACCTTCCA | 174 |
| | 232640_ | TAAACAGATGATTTCAAGATCCCTGGTTAGACACCTCCC | | TTTTGATACTTTACAGTTCTGTTTTCTGAATGACTTTTA | |
| | 992 | ACCTACCTTCATGCAAATTTGAAAAGTACATCCATTTCT | | TTAAATCACTGAGTTTTTCAATTATACCAGTTTCTATGT | |
| | | CAAGAAAACTTTACTTTTCCTCTCAGAAAGTTATCACAG | | TTTCCCACTTCAATAAATTTAGTTTTAACACTTATTTTT | |
| | | TATGGGGGCTTTGCTGACACAAGATATTTTGTTATCATG | | GTACTTAAGAATCTTGACACTATTTGTTTATTTCAACAC | |
| | | TGTTAGAATGTATGATAAGTCAACAAATCACTGGGGTGT | | CTTTAACATCTTGCTCTATTTTTTCCCCTATTATTTTTA | |
| | | GAACAGCCTCCCCAGG | | TTTCACAGATATTTGTA | |
| 349376 | SCAFFOLD | ATGCATATTTATAACCCAATGATTCCTCTTCTTGGTATA | G/T | TGATGGTGAATGAGAAAAGTCAGACACAAAAGAGTACAA | 175 |
| | 26010_ | TGCACAATAAAAATGTGTATATATTTTTACCAATAGTCT | | ATTGATGATTCAGTCCAGTTCAGTTGCTCAGTCATGTCC | |
| | 6081 | AAGAATGTTCACAGCACTGTTATCTTTAAAAGCCTCAAA | | GACTCTGCAACCTCATTGACTGCGGCACTCCAGGCCTCC | |
| | | CTAGAAACAACCCAAGTGCTCATCAACAGTACAATGAAT | | CCGTCCATAGCCAACCCCCAGAGTTTACCCAAACTTATG | |
| | | AAATAAATTATGGTATATTTACCCAATGGAATTTTATAT | | TCCATTGGATGATTACAATAATACAGTTGAACATGTAAA | |
| | | AGCAATGGGAATGAACCAATTACAACTACATGCAACAAT | | AGTAATGTGTGGCATAATAAGTCAGGATGGTGATTACAT | |
| | | ACTGAAGGATCTTGCA | | TTGGAAAGGAGGCAGC | |
| 350721 | SCAFFOLD | ACTTTATTAATAGACACACAGACACTTGTGAGTATTGCA | T/C | GCCTAGGAATCAGTATTTTTAAATTTTTTATAGCTGATA | 176 |
| | 146466_ | GATCCTGCTTTTCTAGATGTAGGATGGAGTTGAGGTTCT | | AGGGCTTCCTAGGTGTCGCTAGTGATCAGGAACCCACCT | |
| | 8003 | GCATTACCTGCAAGTTCCTAGATGAACCATGCTTCTGGT | | GCCAAGCAGGAGACATAAGAGACACTGTTGCCATCTCCA | |
| | | CCATGGGCCACTCTTTAAGAGTATAAAGAGATGGCTCCC | | GGGGTCAGGAAGATCCCCTGGAGGAGGTTATGGCCACCC | |
| | | AAACTTGGTTGCGTATTAGAGCTGCTTGGAGAGCTTTTA | | ATTTCAGTATTCTTGCCTGGAGAATCCCATGAACAGAGG | |
| | | AAAATCAGGATGCCAGGCCACACCCCTGACCAGTTAGAT | | AGTCTGGTGGGCTACAGTCCGTGGGGGTCATACAGAGTC | |
| | | CAGAATCTCAAAGTGA | | GGACACGACTGAAGCG | |
| 346682 | SCAFFOLD | AGGGAGACAGGCGCTACTGATAACAGCCTCATCACCATA | C/T | GGCTGTGTGACCTTCGAGGAGCTGCCTAACCTCTCTGAA | 177 |
| | 270033_ | ATCACTGTGACTGAGTGTCTTTCATAAGCCAAGTGTTGG | | TCTCTGTTCCCTCATCTATGAAATATGGCTAAAGTTACT | |
| | 18768 | CGACTGTACAATAATGCAAACAGGCATTATTATCCCCAT | | ACCTGCCTCACAGATACACATCAAATAGCCTGGCATATA | |
| | | TAGCAGCTAAAGAAACTGAGACTCAGAGAGGCTAATGTG | | GTCTAAATGCTAGGCATTTATTAGATATTGGAATTATTC | |
| | | ACTTTCCTAAATCTCCTAGCTAGAGAGGAGACAGCAGTG | | AAGTAGGAGGACAGGACTTGGTGTCCAAGTCTGTACAGC | |
| | | TATAAATTTCATATATTTAGGTTCTGCCTGTAAATGCTG | | AAAAGCCTGCATGAGTGCTGTCATTCTGTGGTTGTTTGA | |
| | | AGTTCTCTATTTAC | | GACCATAGAAAGCCTC | |
| 348436 | SCAFFOLD | AGACCTTGAATATGCAAGTCCTTCTAAACCTTCCCAAAT | A/G | TTCTAAGGTTGCTAGACAAACAAGAATGTAATCGAAGGC | 178 |
| | 236528_ | GAAAACCTTTCAATGACCATGCCAAGAAAATGTGATTTC | | CTAATTACCTAAAATATTTTGAAATGGGTGCTCAACTGG | |
| | 4224 | TTCAGAAACCACCTCCACTATGCTCAAGGAGGGTTAAAT | | AAAATAGGATGTAATGTCATGGTTTGAGCACTTGGGAAG | |
| | | GGCAGCACAGTTCAGATAGTAAAGTACAAGACATGTGCC | | AGGTATTAATATTCAACTAGGTTAGCCAATCTTGACAGT | |
| | | TGGAGGAGACAGATTGTTTGCCAAAAGAAGTGCAGGATT | | AAGCTATAGCCAGTAAGACTGAAATATCAGACATGCTAT | |
| | | TCATTAACTTACATCAACTCCTGTAAAGAGTTCTGGGGA | | AGACAGAGGTCTAAATGCTAAAGAATGTGAGGATGCTAA | |
| | | ACATGCATTTAACTAG | | AAAGGATCTATTATAT | |
| 344696 | SCAFFOLD | CCCTAACTGAAAGATGGGAGTATTATCCCACCTCAAGAG | T/C | GCATCCCCCAAATGCTCTGTATGTTTTGTGTCAGACACC | 179 |
| | 165011_ | ATTATTGTCGGAATTGAATGAGATATATGCAAAGAGTCA | | AGAGCCATTCTGGAGAAGGAAATGGTAACCCACTCCAGT | |
| | 13796 | AAGTGTCTAACACATAGGAAGTGCTACTTAAAGATTAGC | | ATTCTTGCCTGGAGAATCCCATGGACAGAGGAGCTTGGC | |
| | | TATTATTTCATCAGAGAGCTCAGACTCACTGAAATCAAA | | AGGCTACAATCCATGGGGTCGCAAAGAGTGGGACATGAC | |
| | | TAGCTTGCACAAATCCACACAGATAATAAGACAGAAACT | | TGAGCAACTAACACCCTTCTCCAGAGCCATTCAGTTGTA | |
| | | ATAATTTGAGCCAATGTGAGTCACAGCTGGTTCCCTTTC | | GAACAGGAGAGATGAGACTCCAGCTGGCCTCAGCCTGCC | |
| | | CTCTTCACCAGGATAG | | CCTTCCTCTGGGAGAA | |
| 349604 | SCAFFOLD | TTGTGAACCTTAAAACTAGAACTCTTTGCTCCCCTAAAG | T/C | GGGAGTACCAGTGATGGACAGGGAAGCCTGGCGTGCTGC | 180 |
| | 45297_ | TTAGGCTCAACCTCCCATCCTAGTATTAACTCAACAATA | | AGTCCATGGGGTCGCAAAGAGTGGGACACAACTGAGCAA | |
| | 15085 | ACTATCACTTCCGAACTCACTATGGCTGCTGTTCTGTTT | | CTGAACTATACTTGTTAAACAAACCAAAAAAAAAAAAAA | |
| | | CAGCATCTAGAAAAGAAGTAATGAAAGGCTTCCAGGTCT | | AACACTATTTGTAGTATTATTTCTAATTATATTTGTGTT | |
| | | GTCTTCACCACCTACCACCTGATTAAACCTCTCTGACTC | | TTAAATGGATCAAGTATTCAAAGGGTTCAAAATACAAAA | |
| | | TCTAACACTCAGTGGGAATAAAGTGCCTCCTAGTTACAT | | GTTATTTAAAAGATACAAAAAGTCTCCCTCCCATTCAGT | |
| | | TGCTGTAAAGATAACA | | TACAAGTATTGTATCG | |
| 347934 | SCAFFOLD | AGCTTAGAAAGAGCTTAGAGAAACTTAGAAAGAACTTCT | C/T | GTAACTGTTGGTGGGTCATCTCTCCTCTTGGAGCCTCAA | 181 |
| | 8758_ | GGTGATGAAAAGGGTGGGCAAAGAATCCTGACCCTTGCT | | GCTTTTCATCTCAGTTGCCTAGCATATCAGGTTCACAGG | |
| | 12645 | CTATCATCAGCTCTCTGAGAGACCAAGCTTCATGCTCTC | | AAATATTTGCTGAATCAATGGGAAAATAGGGGACCATGC | |
| | | TCTGGGCCTCAGTTTATTTTCAGTGAAGTTTGAGTGTCA | | TACCTATCTTGGCTGCCTCCCTGCGTGATAGCAAGGATA | |
| | | GGTTAAATGCTCTCACCTTCTTTGGGCTCTTATCCTTCT | | TAGTAAGATAATCCTACACAGTGCCAACTCGAAGAGTGG | |
| | | GAATCTATGGATTGGACAGAACTGGGTGTTCTCTGGTTC | | GTTGCTATGATTCAGCTGGATCCAGGACTGGGTTAACTT | |
| | | TACCTCTTACCAGCTT | | GTCTTCCCCCAGCCCC | |
| 353490 | SCAFFOLD | CAACTAAATAGCAATTATGGTTTTCTGAGCTATAACTTG | G/A | GTATTTAGGTTCTATTCTTATTTTCTGGCTTGTTTGTTG | 182 |
| | 15117_ | AGCACAACTGTTATCATCCTGAGTCTTACTGCAGCCATT | | CTAATTATCATCCCCTTATCCAAATGATGGCATGTTATT | |
| | 27374 | TGTAAGTTTCAAGTGCACACAAGAACATCATTTATCTCT | | AATTGCTTCTTTCTGTGTTGTTGTGCAATGCTAATGCAT | |
| | | TTTGGCAGGCAAATACTTTACCGCCCTGCAGTTAAGATT | | TGTGGAATTCAATAAATATTAAATCAGCCAATCAATCCT | |
| | | ATAGATACTTCGTTTTAGAGCTGTTCATTTCTGTTCTTT | | TTTGGTTAATTTAGTAGATTCATACCTATTCTTTCCAGT | |
| | | GCATCAAAATTTCAAGGATACTAATAATAAGCTAACAAA | | AGGTTTTTATTAATAAAGAATAATTGATATAACAAAATC | |
| | | ATCGTTGAAAGAATTT | | ACAAATTTAAAGTT | |
| 354173 | SCAFFOLD | GTGTCGAACTGAGCAATAAGTGTAGAGGTGCATTATTCT | C/T | ATTATCTCAATTATGTTATTTCTACATAATCATATCTCC | 183 |
| | 260326_ | AATTATGTTGACAAGTGAATTTGTAATCAATGAATTAAT | | AAATTTATGGTGAATCATGAAATTAAGTTCAATGAAATT | |
| | 4125 | TCAATTTCAGATAGCTAGAATCAAAAGTATGGCTTTACA | | TCTTCTGCAATTAAAATGTACTTTTTTATTGTTTACCTT | |
| | | TAAAATAAAAACTCAAGGGAAAAATATGAACTACTAAGA | | TTGTTGTTTTGAGGTTTGTAGATACCTTGAAATAGGCCA | |
| | | GCACTGAGAATAACTAATTGGTATTACACTTTCGACATT | | AATTGCATAAAAAAAAAAAATGCAGTGAGTCTTCTTTTA | |
| | | ATTTTCAATAGCAATTTGTGTTATTTAAGCATTTTGTTA | | GTCCAAAGGAAATACCAGCATGTAAAAGTTTATTCAAAA | |
| | | TTTAAGTATCCATCAG | | GTTTACTCATGTTTC | |
| 351064 | SCAFFOLD | TGATGTGATCTCTCAGATACCTCTTAGACTATGGGGACC | G/C | TAAGGGCCAGGCATGTGTCAGAACTCCATCTAACATCTG | 184 |
| | 295006_ | AGACGGCCATCAGATTCCTTGAGGGCCAAATGGATTGCG | | TCTTACCATTTAGTCAGTGCTTATCTTTTCCAATAAGCC | |
| | 45401 | AAGTTCTCACTGGCCTTTACAGCAGCTACAGGCTCTAAA | | CATAAGGGCAGAGGCCATATGTACACTGCATGCTGTTTT | |
| | | TGTCACTCCCCTAAGGCTCCATCCTGGGTTCTTATTCCC | | AACTGTAGTCCCTGATATAGAACCTGGAATCACAGGTGC | |
| | | TCTAGTCCTCCTGTAGCTGCCCTATGGCTCCCTATAGTA | | TCAATAAATACAGGGTGAATTAATCTAGACCAACCCCCT | |
| | | GAACTGAATATGGCTTAAAGTATGTATTTATGTGATTAA | | TACTTAAGAGTTGAGCGTACTGAGTTTCTGAACAATTAG | |
| | | TTTCATTAACTATTTG | | ACATGTAACCCAAGTC | |
| 342761 | IBISS4 | GCCCTAATCACACAGACAAGGCTGGTGCCAGCCTTAGGC | G/C | TGTTCACCCTCTGGCCAGACGACTGTGGTTCAAGACACA | 185 |
| | snp758 | TTGACACGGCA | | TGTAAATTGCT | |
| 345383 | SCAFFOLD | ATAAAAATTTCTTTTATCGATCCTCCCATTTTCTGAACT | C/T | GCACTTCAGAGGAGCCTACTTCTCCCTTTTCCACCTTAA | 186 |
| | 115964_ | TCTAAATATTTAGAGTGAGTAGTATTCTAGTTCCCAATC | | TTCATTCATTAGTGGTGGTTTCCTTTGGTCCTCTGGATT | |
| | 5023 | AAATACACATTTGGGTACCATAATTTGTGTTTTCTTTGT | | TCCTTTCATCAGTGAAGGAGCACCATCTGGTAGCAGACA | |
| | | AAAGGTATGTTGATTTTTTTTTTTCTTTCTTTCAAAGGA | | ACAGTAAAATCAAGCTACACAGAACACACGCAGGGAGGA | |
| | | ACTCTTTAAAAATATGATAATTTTCTTCTAGGATAATGA | | CTCCCTTCAACCTCCCTCCTCCCAGCCAACATCTGCTTC | |
| | | TGTTTTTGAATGCCACACAGAAAACAGCCTCTTTCTCAC | | CCTCTGCTGTTTGCAGTGACATTTTAAAGGGAAAACTGG | |
| | | CGATTGTATTTCTGTAAA | | GAATCATGGTAGTAAT | |
| 345918 | SCAFFOLD | CCTTCAATGCAGGAGACCCCTGATCGATTCCTGGGTTAG | C/T | ATACACGACTGGGTGACTTGATCTTTACCCCAAATCATT | 187 |
| | 305304_ | GAAGATCAACTGGAGAAGGGATAGGCAACCCACTTCAGT | | CAGGCTTCTCTGGTGGCTCAGTGGTAAAGAATCCACCTG | |
| | 4542 | AATCTTGAGCTTCCCTTGTGGCTCAGCTGATAAAGAATC | | CAATGCAGGAGCCACAGGAGACACAGGTTCAATCCCTGG | |
| | | TGCCTGCAATGCAGGAGACCTGAGTTTGATCCCTGAGTT | | GTCGGGAAGACCCCCTGGAGGAGGGCATGGCAACCCACT | |
| | | GGGAAGATCCCCTGGAGAAGGAAAAGGCTACCCACTCCA | | CCAGTATTCTTGCCTGGAGAATTCCATGGACAGAGGAGC | |
| | | GTATTCTGGCCTGGAGAATTCCATGGCCTGTATAGTACA | | CTAGTGGGCTACAGTCCAGGTCACAAAGAGTCGGACCGG | |
| | | TGGGGTCACAAAGACT | | ACTGAAGCAGCTTAG | |
| 346960 | SCAFFOLD | GCTACGTGCTCCTGTATTTTGTCTTTTCTGTTTATCCTC | G/A | TTAACAAACAGGTCGTCAACCCATGTGTTCTGAACTATG | 188 |
| | 70112_ | ACTTTTGCTATCTGGAAATCAAAGTTACACCCTCAGAGG | | CAAGAAATGCAGACTGTTTCTTTACCTGGGTACACGGAA | |
| | 31828 | AGGATAAAGAGTTGGTTTGAAAGCTTCCATCTGAAAGAA | | GAAATTTCACTAGGCATGAAAATAGGAAGATACTTACAG | |
| | | TACGTGAAAACTATGGCTTAGAAAAACCTTCGGATTCTT | | TATTCATCTTAACAATGTGTGTATAGGTGAGTAAAATTT | |
| | | CACCACTGAGCCACTCTAACATGTATGAAATGTCCACAG | | TGACTAAGTCTTAGAAGAAAGAAAGAGAAAATATTTTCT | |
| | | GGTGGAAGCTCTGTGGCAGAACGTCAAGAAAGGGTCTCT | | AAGATAAGTTCATGGTAGCAAAATAAAGTCTAAGCTATG | |
| | | TGTGCTCTGGAACATC | | AAACATTNNNNNNNNN | |
| 346264 | SCAFFOLD | GTCCATGGAGTCACAAAGAGTTGGACATGACTGAAGTGA | T/C | GCAACTGTATTTTTTGCCCTGCATAAAATAGCAAAAATA | 189 |
| | 112150_ | CTTAGCACATGCACCCAATCACCAATGTGAAAAACGAAA | | ATTAAACAAGTTCGAATAATCAGGGCTACTTTGACAAAC | |
| | 2535 | AGCACTGGGCAATGATCTGGGGCAAGAAAAGACAGAAGG | | ATAATTATTCCAAAGCCATTATGCCATTACGCCATGTAT | |
| | | AAGAACGTGGGAACAGGAGGAGTTTAAGGCCACAGCCAC | | CATAACACTTTCTTGTTTTCAAAAGAAAATAAAAATAGA | |
| | | GAAGCCATGTTTTTGTTCTTTTGCCACAAAAGCAGGCAC | | GAAATCCTTATATTGGTTTAGCTCAAAGGTTTATCCAGC | |
| | | TGATCATAGAGTTTAAGGCTTAGAAAGCAGGGAAAGCAC | | CTAACAGTGCATGTGACAGGCTCAAAAGGAACATCGTAT | |
| | | CTGTCAATAAGGACCA | | GGAAGGGCCAAGTTTT | |
| 342912 | SCAFFOLD | GGACACAGGGCATTTCAGAAAGGAATGAGTTTATTAAGA | G/A | ACTGAAGTGATGCAGCAGCATATCCTTGAGACAAAGAAA | 190 |
| | 18551_ | ACAAAGAGCAGAGTTAATGGGGACACTGAGAGCACAGTG | | ATTCCTGCTGGAAGACTGGCGGTAGGTGATCGGTTTGGG | |
| | 1128 | GGCCAACCTCCTGATGGACCAGGGAAAGCCAACAATTTT | | GCACTATAGGGTGTTTACTGGAGTCGGCATCTTTGCTTA | |
| | | TGCGAATAGCCAATGTTTATATCCTTGAGTGGAGAAGGA | | ACCAAGAGTCAGGAAGTTTGTTAGTGATGATCAGGAGGC | |
| | | AATGGCAACCCACTCCAGTGTTCTTGCCTGGAGAATCCC | | TTTTGGCAATGGCTACAAAGGGGCTCAGTCAGCTTCAGA | |
| | | AGGGATGGGGGAGCCTCGTGGGCTGCCGTCTCTGGGGTC | | GATGACCTTGGTTCTGGGCTCCACTTCCATGGTTTTGGT | |
| | | ACACGGAGTCGGACAC | | GCAAGGCCTTGCACCTG | |
| 345811 | SCAFFOLD | AACAAAGGCATGTGTAGTCAAAGCTATGGCTTTTCCAGT | A/T | CCAATACCTTGGCCACTTGATGCAAGAAGCTGACACCTT | 191 |
| | 264251_ | AGTCATATATAGATGTGTGAGTTGGACCATAAAGAAGAC | | AGAAAAGACCCAGATGCTGGGAAAGATCGAATGCAGGAG | |
| | 648 | TGAGCACGGGAGAATTGATGCTTTTGAACTGTGGTGTTG | | GAGAAGGGGAAAAAAGAGGATGAGATGGTTGGATCGCAC | |
| | | GAGAAGATTTTGAAAGTCCCTTGGGCTGCAAGGAGATCA | | CACTGACTCAATGGACATGAGTTTGAACAAGCTCCAGAT | |
| | | AACCAGTCAATCCTAATGAAATCAATCCTGAATATTCAT | | AATTGTGAAGGACAGAGAAGCCTGGCGTGCTGCAGTCCA | |
| | | TTAATGAATATCAGTGAAAATCAGTGAATATTCATTGGA | | TGGGGTCACAAAGAGTCAGACACAACCGAGTGACTGAAC | |
| | | AGAACTGATGCTGAAC | | AACAACAACTCTCTAT | |
| 352161 | SCAFFOLD | CAAAGAGCTGGACACGACTGAGCACACATTTATTTATTT | C/T | TTTACTCTGTAGTCCATATTGTTGATTCACGTGGAATGG | 192 |
| | 205065_ | GGAAGTGCTGGGTCTTAGCCACTGGATCACCAGGGAAGT | | GAGCCTCGCAGAATATGCCTTATTTTCCTTGGATTGTGT | |
| | 7302 | CCCTCAGTACTCAACTTTGAATTGTTTTGCAGTCCCCTA | | GTTAATTTTGAAGTCTGTCTGTGTGCTAAGTTTTGGTGA | |
| | | TCAGCAGTGCCTGCTTTGAGACTTCAGAGGCATCTGGTA | | ACATATGCTTCAGAATGGAAATGTCAGTCCAGAAGATAA | |
| | | AACCACGATCTTCTGACTTGCAGAAGGTCTTTTCACATC | | AAGAGAGATAAAAGTTGAAGATTGAAAGGTGGAAGTCAT | |
| | | CTTGGAGGCCTTGGCAATTGAATCCTACTCTTCTTCCTA | | ATGCTCTCCAACTGGATAAGAATCCCAAGGGAAAGAATG | |
| | | GTGTAAACCTTTGTTA | | TAAAAAGAGAAGACTG | |
| 352870 | SCAFFOLD | CAGCCATAACTTCCTTTTTGCCCTTTGGCTAGGCTGCCT | T/G | AACAAATGGAACTAGAATAATTCACCAAATCATACACTT | 193 |
| | 305349_ | AAGGAGGAGGACCTCGGCCAGTTCTGTTCTGGAGGTGAC | | TATTATTTTTGAACAACCGCGTCTCCCACTTGACAGTAA | |
| | 22810 | ACACAGATGGATGCTGGCAGGAAGCTGAGGACCAACACT | | GCTCCATACAGGTGATAAACATGACTCTCTTGTTCAGTG | |
| | | CCTCAAGGCTGAGGGCTCTGTGCACCCCTGACCAAACTT | | TTGTAGCTCCAGTACCAAAAGGTGCTTAGCGAGTGACAG | |
| | | CTGATGCAAAATATTTACACTAATGTCACACATTTATAA | | GGGCTTGGCAAATGATGACCACAGCTGACACAGACCAGT | |
| | | AAAACAATTGCAACTAAAGATATCTTGCAGCAACTTTTA | | GGTCAGCTCTTCCAGGCACATGCTTTCACTTTCCATGCT | |
| | | AACAATGCTCTACAAA | | GTCTCTCAGGTAATCC | |
| 353614 | SCAFFOLD | TCATTCACAGCCCATCTGAATAAACACCAGACGTCCAAT | G/A | AAGAATGAGAGTTATAATATGCAAATATATCATATTAAT | 194 |
| | 285370_ | CTGTATACATTTTGCCTATACAAAGTCTAAATCTTAGTA | | GGTATATTATATTTATTTGATGACATCTTTTCCAATTAA | |
| | 4116 | ACAATACTGAGCTTATGTAAAACTGAACTTTTCATTCTT | | ATAGGAAGAATTACATTTGAACAAATCTCATAAATTAAA | |
| | | AACTACATGTAAAGTAGACATGGCTTTGTGTTCTCTCTT | | CAATGCCTTCCTCTGTTTTGTTGAAATTTTTAATAATTT | |
| | | TTTGTGTCAAAACCAATTATTAGGACATCCTAAGCTGTC | | TATTTGATTAGTGGGTTACTATTTAAAGAATCCTCAAAC | |
| | | TTTCTTAGTGGCAACTTAGCCTTTACTTTAAAAAAAAAA | | ACTATAGCTTCTGTCTATACAATTGATTTGTTCATTTTT | |
| | | AAAAAGGTTCCTGTAA | | TTATGAGATCAATATA | |
| 348400 | SCAFFOLD | CAGAGCATATGAGGACTCCAGGTAGACAGAATTTGGCTC | G/T | GAGAAGAGTCTTCATTTCTGCTTCTAAGAGGATAACATT | 195 |
| | 221545_ | AGAATGCTCCCAGTGGGGCAGGTATCTTCTCTGTGTGTG | | TACCTCCCCATTGGTAAAATTACAAATCAGTGTATTTGA | |
| | 4766 | CTGGACCATGTCTTGTCTTGCTTGGCACCCTCCCCACCT | | AAACCACTTTGCAAAGCACTTTCCTTTTCTCTCAATTCA | |
| | | CATACCTTGCTGTCTCCCCCTCCTCTGTGGTCATGAACA | | GTCTGACAAGTATTTCGTCAGCACCAACTTGGTGCCAGG | |
| | | TGTGCCCACCCTGCTCTCAGCATCTTGATGCATTAACCA | | CATCTTTATAAGGAATCTGTGCAGGAGGTGAGGCGCTGA | |
| | | GAGGCTTCCCCTACCTCTCCTGACTTATCCCCCATCCAA | | GAATGGTACTGAAGTGCCCCATGGCTCCTGACAAATTGG | |
| | | CCACAGACTTTTTTCC | | GGACTGTGCTCAACCC | |
| 348888 | IBISS4 | GCATCCTACATTTATCCTGTGTTCTGCCTTTGTTTTAAT | T/C | TACAGCAGAAGGGTTCTCTGGGTCCCCAGTTTCCAGTTG | 196 |
| | snp785 | TTTGACTCAGC | | ACAGTATATCC | |
| 346608 | SCAFFOLD | TCCCTGGAAAGGTGTGTCGAATTCCAAACCAGTGATACA | C/T | CCTCTGAGTTATATTCTCCCAGCTCAGTTCCATGATTTT | 197 |
| | 242466_ | CAAACAAACCTTTGAATCATGACCCTACAGAAGCTGGAG | | GTCTGAAGTAAATGCCCAAGGTAGCAGATAATCCTGCTC | |
| | 580 | AACTGTCTGTTCTTTTGACTTGTCCTTGAGCTCTTTGAG | | TACTAGGAACCTGCCAGTGATGTCTTCAGCCAACATTCT | |
| | | GCATCCCAACTGCCCTCCTAGTGCTACTCTTCTGGGATT | | CTTCGTAGGCCTCCAGTGGCTATTTGTATCTGCATAAGA | |
| | | GTGGGACTCTCTGTTTGCCTTATGATGGTTTTCTAAACT | | AGACTCCTGAAAGTCCCTTGGACAGCAAGGAGATCAAAC | |
| | | TCAGATTCCAACAGGGATTTGACCAGACAGGAACAAAGA | | CAGTCAGTTTTAAGGGAGATCAACCCTGTATATTCACTG | |
| | | GGACCTGAGATCTGGT | | GAAGAACTGATGCTGA | |
| 348360 | SCAFFOLD | CCTGTATGGAGCCTTGTTTCTGGCTCCTCTGTGGTGCCA | C/G | CTAAGCTAAAATAACAAAGCAAAAACAAAACCCCAATTA | 198 |
| | 210144_ | GAACAGAGTTCCTGCCAGTCAGCCGGTCTGGGGCTTAAA | | TTTTTTTCCATAGAGAGAAAAGACTAATTTTCATTTGTG | |
| | 9408 | GATTTCTTCATACTGCAGCTCTGCAAGCCTAAATGAAGA | | TGATTTTGAAGGACTTAGTTAAATATGGCACTGCCAGTC | |
| | | GCAGATTATAGAGTCTTAGACAAATAAAAGGAATACTGA | | AAAGCACAAATTGAGAGACAGAATAACACTTCCATTTAG | |
| | | GTGGTTATAAAATCTTCCTTTTTTGACTTGAAAAACAAA | | AAAGGTATTTTCAGATTCCTTGTACTAAATTTTACTCTG | |
| | | TATGCCTGTATTGTCAGGCTGTGTGTACATTGATGCAGG | | TATGTTCAGCAGTGGCTGCGTAATTATTGCTTCCATGTA | |
| | | CTTGCATAATACAGCT | | CTGACATGCCAGAGTT | |
| 348820 | SCAFFOLD | GGGCTCAGTGGTAAGGAACCTGTCTGCCAACTCAGGAGA | G/A | TTGCCTGTGTTTTCAGTTGGCTTGTGTTTTTATTATTGA | 199 |
| | 80920_ | CTGGGGTTTGATCCCTGGATTGGAAGATCCCCAGGAGAA | | GTTGTAGGCGTCTCTTCTGTATTTTTTTTTTTTTTGGCT | |
| | 8235 | GGGAATGGCAACCCACTCCAATATTCTTGCCCGGGAGAT | | GTGCTGCGTTGTCTGGTAGGATCTTAGTTCCTTTCCTGA | |
| | | CCTATGGACAGAGGTGCCTGGCGGGCTACAGTCCATGGA | | CCAGGAATTGAACCTGGGCCCTGGGCCGTGAAAGCACAC | |
| | | ATGGCAAAGAGTCGGACATGACTGAGCACGCACACACAT | | AGTCCTAAACACTGGACTGCAGGGAAGTCCCTCCTTCTG | |
| | | TTTTCCATGCACTTATCAGCCATTTGTACATTTAGAGAA | | TATCCAGATACAGATCCCTTCTCAAGTGTATGGGTTGCA | |
| | | TTGTCTATTCAGGTCT | | GAGGTTTGCTTCCGTT | |
| 347423 | SCAFFOLD | TGACTGCGCCCAGTAGCACCCATGAGCATGCTTTCTGCA | T/C | GAGTCCAAGGAGAAGTCTTTGAGAGCTAATGGAGAGGAG | 200 |
| | 216765_ | CTCTGTTTTGAGCCCCCTCTTTCTTTCAACCCTATTTCC | | CCTCCTCCATCTCAGAACCACCTACAAAACAGAAACTGA | |
| | 5028 | TCACCATTTAAAAAAGTCTTGTAATGTGTATACTTTGAG | | AAATATACTTTCCTTCCTCCGAAGATGCCCTCAGCTTGT | |
| | | AAACCTCCTTCAGTTCTTTCTACTGTAGGCAGGAAACAC | | TCTTCAATGTGTTCAAAGTCAAGAGCACCTGAAGAGTTA | |
| | | ATATATATGAAGCCAACTAAGATTACTCTGACCTGAGTT | | GAGAGAACAGAACTTTAGATGCAGGAAGTTATGGAAATG | |
| | | CTAGAAGTTATAAAGTGAGGAGGCAAAGAACCTAAACCC | | TGCTGCCTGTGAACTTCCCCCCCAAAGAGCCTTGCCTCT | |
| | | AAGGACTAAACCTTTC | | GCTCATGGATTAACCT | |
| 349603 | SCAFFOLD | CAAATGTACATGAAATAAATCATGTTAAAAGCAATGCTA | T/G | GTTCTAAAATCAGGGCTTAATTTATTGCTTTGTTGACTG | 201 |
| | 45055_ | ATAAATATTCAAAACTCAATCGCTTTCTAATTATTTTAG | | TATACTTGGAGGTCACTATGGCCAGTAGGCCAAATTCAA | |
| | 25659 | AACATTCTACTATTATCAAGGTTCTTGGGGCAATTTTTG | | CCTCAAGTTTACTGAATCACAGCTTTTTTTTTTTTTTTT | |
| | | TCTGCTGGGTCTATATGGTGGTAAGACTATAAAATGGCG | | TTTTGACATACTGTTTATGGATGCTTTTGTGCTATAATG | |
| | | TGCTGCTCTACATCTCTTGTCAAACTGGCATTAATCACC | | GCCAAGGTGATTAGCTGCAATAGATACCATATAACCCCA | |
| | | ATATCAACACCATGAAATTGGCTAGGGTGGGAATACTCA | | AAAGCCTGAAGTATTTCCTATCTGACCCTTAGAGAAAGT | |
| | | CATCATGAAACTTGGC | | TTGCTGACCTCTGGCCTA | |
| 350976 | SCAFFOLD | AAGATTTGTGTCATGGATGTCCAACATACTCATTATTTT | A/G | TAAAGACCCCTTAATGCAGGGGAATGCAAAGCACATGGA | 202 |
| | 256426_ | CCAGATTTCCTTTTAGCCTTCTTCCTCTTCTCTGACATC | | CTTTAAACAAAATTTCAACTCTGATACTTTAATTTATGG | |
| | 385 | CCTGGTTGCCAAAAGATCTTCAGTTTGGTAACCATCCAG | | CTGAGTAAATTACTTAATAAATGATCAGTCTCAGTATTA | |
| | | GCACATGCCCTAAATTCTGAAAATATTCTGATCACTAAT | | GGCTCACCATGTTCAATCATGGTTTTAGGTGCTTGGTGC | |
| | | ATATATTACCCCATTCCCCATTTAAGTCTCTTAATGTTA | | ACTTCAATTTATGTAGTTCTCATAACAACCCTCTGAGAA | |
| | | TTGCTTAAAATGCAGTGTTTTTTAAAGAAGCCCTTAATG | | GGTCCTAATTATGACAAGAGAATTGAGACACAGAGAATG | |
| | | TAAGACTGTATGTAAT | | TAAGTTACTTTCCCAG | |
| 343688 | SCAFFOLD | TCCATTTCTAGGGCTTTTTTTTTCCCCAATAGTAACATA | A/G | GGAAAGGGAGAACTGTCCAGAAAGAAGGAAACCTGTGAG | 203 |
| | 70184_ | TGAAGGAATGGGCTGACCTTTAATTTATAATCCTCGTGT | | GTGGAAGAAGTGCTGGAAAACGAACAGGAAGATGATAAA | |
| | 5981 | CACGGAGTTTAGCCGAGCTTAACCTATGTCAGTTGTATT | | GAAAAATTTTTGCAGCACTTTGTAACTGGAGAGCGACCC | |
| | | ATAGTTCAAAATCGTCTGCCTTGCTTAGGAAAAGGCAGG | | CATGGACTGTAGCCGCCAGGCTCCTCTGTCCATGGGATT | |
| | | CTGTGCTTTCAGTGTCAGGTTGAAAAAATATATAAATAA | | CTCCAGACAAGAATACTGGAGTGGATTGCATGCCCTCCT | |
| | | AAAAAGAAACGTGGGAAAATAGACCTTCATTAAAAAAGG | | CCAGGGAATCTTCCCAACCCAGGGATCAGTCCTGGGTCT | |
| | | CAGAGGAAGGCTCCAC | | CCTGCCTTGCAGGCAC | |
| 349988 | SCAFFOLD | GTCTTTATTCACTAGAGATACTTACCTGTAATTTTATTA | T/C | AGACTTTCTATTTCTGCACAATTTAGTCTTAGAAGATTA | 204 |
| | 179015_ | TTTGTAGTGTCTTTGCTTTTTCTATTGGAGTAATGGAAG | | TCTATTTCTAGGAATTTTTACTTTATTCTAGGTTGTCCA | |
| | 1131 | GAGTTTGAAAGTGTTTATCTTCTATATTTTTTGAAAGGT | | GTTTGTTAACTTTATTGATAGTAATCCAATATCATCCTC | |
| | | TTGGGAAGGATAGGTGTTGACTGTTCTGTACTTGTTTGG | | TATATTTCCATGATGTTCTTTGCAATTTTTTGTTGTTCA | |
| | | TGGAATTCTCTCATGAAGCCTTTGGTTCCTATACTTATC | | TTTCTGATTTTATTAGTTGAGCTCTTTTTTTTTTTTTTT | |
| | | TTTATTTGTCGTTTTTGATTACTGATTCAATTTCATTAC | | TTCCTTATGAGGCTTGCCAAAGTTTCATCACTTTTATTT | |
| | | TAGTGATCATTCTGTT | | TTACCAATAGTGGAAG | |
| 352122 | SCAFFOLD | CAATATATTGTCGAAAAACTGTTTGTCTTTATTCACTAG | T/C | TCATTACTAGTGATCATTCTGTTTAGACTTTCTATTCTG | 205 |
| | 179015_ | AGATACTTACCTGTAATTTTATTATTTGTAGTGTCTTGC | | CACAATTTAGTCTTAGAAGATTATCTATTTCTAGGAATT | |
| | 1107 | TTTTTCTATTGGAGTAATGGAAGGAGTTTGAAAGTGTTT | | TTTACTTTATTCTAGGTTGTCCAGTTTGTTAACTTTATT | |
| | | ATCTTCTATATTTTTTGAAAGGTTTGGGAAGGATAGGTG | | GATAGTAATCCAATATCATCCTCTATATTTCCATGATGT | |
| | | TTGACTGTTCTGTACTTGTTTGGTGGAATTCTCTCATGA | | TCTTTGCAATTTTTTGTTGTTCATTTCTGATTTTATTAG | |
| | | AGCCTTTGGTTCCTATACTTATCTTTATTTGTCGTTTTT | | TTGAGCTCTTTTTTTTTTTTTTTTTCCTTATGAGGCTTG | |
| | | GATTACTGATTCAAT | | CCAAAGTTTCATCAC | |

TABLE 8
Additional SNP associated with NFI

| | |
|---|---|
| 343193 | IBISS4snp651 |

(SEQ ID NO: 206)

ACACTGGGGCTCCTTTTGCCTTTTTTAGCAGAACGTCCGTCCATCCATCC
A/GCATCTCTGTCCCGTGACTCAGGGGCACCCACTCAGCTTTGATTCTCC
TCC

| | |
|---|---|
| 343617 | SCAFFOLD35407_3401 |

(SEQ ID NO: 207)

TGCACAAGCAGAAAATGTGGTTCATGTCCTGTTGAATTCTAAATATGTTG
TCTCTGAGATTATCTCTCCTATTTCTTGCCTAATTTATCCATCACAATCC
CCAACATGTTTCTTGGTGGCACTTGGTACCTTGTTCTATATTTATAAAGG
GATCCTTTAATTTTTGTTGTTGTTGTTCAGTCTCTCAGTCATATCTGACT
CTTTGTGACCTCGTGGACTTCAGCACGCCAGGCTTCTTTGTCCACCATCT
T/CCCAGGGCTTGCTCAAACTCATGTCCATTGAGTTGGTAATGCCATTCA
ACTATCTTTTACTCTGTCATCCCCTTCTCCTGCTGCCTTCAATCTTTCCC
AGCAACAGGGCTTTTCTAATGAGTCAGCTCTTCCCATCAGGTGACCAAAG
TACTAGAACTTCAACTTCAGCACCAGTCCTTCCAATGAATATGCAGGACT
GATTTTCTTTAGATTGACTAGTTTGATATCTTTACAGTCCAAGGGACTCT
CAA

| | |
|---|---|
| 343763 | IBISS4snp160 |

(SEQ ID NO: 208)

TTAGATGCCCTGGACAAGGACAGCTCGCCAAAGGATGACACTTGGGACTC
C/TGTGTCGGTCGTGACGTTTCCAGAGAATGAGCAAGAAGGGAGTCCCCA
AAG

| | |
|---|---|
| 344483 | IBISS4snp347 |

(SEQ ID NO: 209)

TATTGCTGGACTTCTGTTGTAACAAGTTGGCAAACACTGGCTGGAACTGG
T/GCTGCAATAAAACATGCCAGTATCAATGCTGACAAGAGCCTAACAAGT
GCC

| | |
|---|---|
| 347069 | IBISS4snp426 |

(SEQ ID NO: 210)

AGAATCTATGAGAGAGATGACTTCAGAGGACAGATGTCAGAGATCACAGA
C/TGATTGTCCCTCTCTTCAAGACCGCTTCCACCTCACTGAGGTTCACTC
CCT

| | |
|---|---|
| 347999 | IBISS4snp723 |

(SEQ ID NO: 211)

TAACATCTCACTATTCTCCTGTGGTCTGATAGAAACAGACACATTCTTTC
A/TAGAGCAGAGCAGTATCCTGTAATCCTAGACCTTTTCATGACACTTGG
AAA

| | |
|---|---|
| 348798 | SCAFFOLD7023_4924 |

(SEQ ID NO: 212)

CTTCATATTTATGGACTAGACAAGTCTTTCTGGGGCAAATATTTATTTTG
AAAAGTGCCACTTGAACAGACAAAATTAACTTATTACCCAGGTGAAAAAG
GACAATACAAGCTTCTTTGGCTTAAAAAAAAGTTGCTGTTAATCTGATTT
TGGATTGCTGAGTTGATTACTGGAGAGGAGATAGGAATTTTTCCTAAGGA
AGAAAGAATTACCTAAGAGGAAAGCTCAACTCTTTCCTTTTTGAGGCTCA
A/GTCTTATAAACACCACTCTGACAGACCAGATCTTTGAAAAGTCCAGTG
ACATAAAGGTCTCCAATGTCCCCAGGGAGGAACTCACTGTGGGAGAAGGT
AGAATTGAACAGGTGAGCAAAGTGAAAGATTAAAGTGGAAGATTGACTTG
GGGAGGCTTCGACATGGGTTATAATTCAGTGAAGACCAGAAATACATATA
AACCCAACCATTTTGGCAAATTGGTTACGGAAGCAAGTAATCATTAAACT
TGA

| | |
|---|---|
| 349583 | SCAFFOLD35711_6569 |

(SEQ ID NO: 213)

GTCCAGATGCCTTGGCAATCCAGAATGCAGTCAAGAATTAAGTAGGAAAA
AATAATCTGAAAAACTCAGTGATTATTCAAAAGTCTGGTTACTAGTATTT
ATGTATTTACTAATACAAGCCCTTGTTTAGCTCTCATGGGTAAAACCTTT
CTTTTGTAAGTTTTTAAAAAGTTGATATTTGGTTTCTCAATCCACAGTAC
AATGGAAAAAGCCTTGAGGGGAGGGGCTAGGGTGGAGGAAGATCTCAGAG
A/GCTACAGAATCTGTTACAGTATTATTACTGGTTATTAGAAAGTAGATT
AAAACCTACCCAAAGTAGGCAATGGAACAAAAACATAGAAATCAGAATCT
ATTTAGCAGGATATCCTATACCTTAGTCATCTTCGGGGAAAATAATCACA
GTATATCGCCTGACAAAAACATAGTAACCAAACAAATTTTAAATAAATGT
TTGCTATATTAAAATAGAACAAAAAGGCTTAGCGTGTGATTTTTCATGTT
TTT

### EXAMPLE 4

Intramuscular fat is a commercially import trait of cattle that is a prime determinant of the value of a carcass through its surrogate, marbling score. We genotyped pairs of animals of Example 1 for a range of nir fat measurements as described in Example 2. Associations found between DNA markers and intramuscular fat are set out in Table 9.

TABLE 9
Associations between DNA markers and intramuscular fat sorted in
decreasing order of statistical significance.

| Locus | mean_0 | SD | mean_1 | SD | mean_2 | SD | N | Freq | a | k | alpha | tmax | log(1/P) |
|---|---|---|---|---|---|---|---|---|---|---|---|---|---|
| 352076 | 0.18 | 1.68 | −0.13 | 1.42 | −1.75 | 0.63 | 162 | 0.81 | 0.96 | 0.68 | 1.37 | 9.825 | 5.0000 |
| 347422 | 0.18 | 1.70 | −1.51 | 0.55 | 0.00 | 0.00 | 142 | 0.96 | 0.09 | −17.78 | −1.39 | 10.732 | 5.0000 |
| 343362 | 0.14 | 1.61 | −1.52 | 0.75 | 0.00 | 0.00 | 164 | 0.95 | 0.07 | −22.71 | −1.35 | 9.697 | 5.0000 |
| 343054 | 0.47 | 1.83 | 0.05 | 1.32 | −1.03 | 1.24 | 163 | 0.60 | 0.75 | 0.44 | 0.81 | 7.403 | 5.0000 |
| 352376 | 0.23 | 1.70 | −0.49 | 0.78 | −1.31 | 0.96 | 145 | 0.79 | 0.77 | 0.06 | 0.80 | 8.344 | 5.0000 |
| 348950 | 0.00 | 0.00 | −1.45 | 0.45 | 0.09 | 1.63 | 164 | 0.03 | −0.04 | 33.22 | 1.35 | 11.549 | 5.0000 |
| 353833 | −1.31 | 0.90 | 0.12 | 1.81 | 0.24 | 1.50 | 161 | 0.30 | −0.78 | −0.85 | −1.04 | 7.564 | 5.0000 |
| 349339 | −1.31 | 0.88 | 0.56 | 1.75 | 0.05 | 1.53 | 160 | 0.22 | −0.68 | −1.75 | −1.35 | 8.171 | 5.0000 |
| 353331 | −1.17 | 0.89 | 0.65 | 2.21 | −0.13 | 1.27 | 163 | 0.23 | −0.52 | −2.50 | −1.21 | 7.541 | 5.0000 |
| 345497 | 0.00 | 0.00 | −1.41 | 0.60 | 0.12 | 1.64 | 160 | 0.05 | −0.06 | 24.50 | 1.25 | 10.047 | 5.0000 |
| 343610 | 0.00 | 0.00 | −1.60 | 0.87 | 0.13 | 1.60 | 163 | 0.04 | −0.07 | 25.62 | 1.47 | 8.956 | 5.0000 |
| 353107 | 0.18 | 1.68 | −0.16 | 1.41 | −1.75 | 0.63 | 163 | 0.81 | 0.96 | 0.65 | 1.35 | 9.884 | 4.6990 |
| 350939 | 0.00 | 0.00 | −1.02 | 0.85 | 0.15 | 1.67 | 164 | 0.08 | −0.07 | 14.60 | 0.84 | 7.322 | 4.6990 |
| 350910 | 0.00 | 0.00 | −1.53 | 0.81 | 0.13 | 1.62 | 163 | 0.05 | −0.07 | 24.54 | 1.37 | 9.221 | 4.5230 |
| 352223 | 0.22 | 1.63 | −0.74 | 0.94 | −1.77 | 1.02 | 164 | 0.88 | 0.99 | 0.04 | 1.02 | 8.324 | 4.3980 |
| 351771 | 0.19 | 1.75 | 0.33 | 1.35 | −1.06 | 1.30 | 163 | 0.63 | 0.62 | 1.22 | 0.82 | 6.872 | 4.3980 |
| 343786 | −1.26 | 0.90 | 0.29 | 1.89 | 0.09 | 1.53 | 153 | 0.29 | −0.68 | −1.30 | −1.04 | 7.196 | 4.3980 |
| 354364 | 0.10 | 1.60 | −1.49 | 0.90 | 0.00 | 0.00 | 163 | 0.96 | 0.05 | −30.80 | −1.36 | 8.286 | 4.3980 |
| 348335 | −0.91 | 1.19 | −0.33 | 1.26 | 0.52 | 1.79 | 162 | 0.35 | −0.72 | 0.19 | −0.67 | 7.190 | 4.3980 |
| 353963 | 0.08 | 1.61 | −1.69 | 0.62 | 0.00 | 0.00 | 164 | 0.97 | 0.04 | −43.25 | −1.60 | 10.306 | 4.3980 |
| 352880 | 0.00 | 0.00 | −1.30 | 1.01 | 0.17 | 1.60 | 164 | 0.06 | −0.09 | 16.29 | 1.12 | 7.810 | 4.3010 |
| 347009 | −0.28 | 1.36 | 0.57 | 1.84 | −0.88 | 1.13 | 161 | 0.64 | 0.30 | 3.83 | 0.63 | 6.948 | 4.3010 |
| 348491 | 0.67 | 1.82 | −0.56 | 1.49 | −0.45 | 1.02 | 164 | 0.56 | 0.56 | −1.20 | 0.48 | 6.244 | 4.2220 |
| 350037 | 0.00 | 0.00 | −1.31 | 0.78 | 0.10 | 1.63 | 163 | 0.05 | −0.05 | 27.20 | 1.18 | 8.268 | 4.1550 |
| 352656 | −1.10 | 1.10 | 0.07 | 1.67 | 0.30 | 1.58 | 164 | 0.37 | −0.70 | −0.67 | −0.82 | 6.838 | 4.0970 |
| 352655 | −1.10 | 1.10 | 0.07 | 1.67 | 0.30 | 1.58 | 164 | 0.37 | −0.70 | −0.67 | −0.82 | 6.838 | 4.0970 |
| 353195 | 0.19 | 1.65 | −0.95 | 1.12 | 0.00 | 0.00 | 164 | 0.91 | 0.10 | −11.00 | −0.75 | 6.441 | 4.0000 |
| 351385 | 0.07 | 1.60 | −1.91 | 0.59 | 0.00 | 0.00 | 164 | 0.98 | 0.04 | −55.57 | −1.83 | 10.922 | 4.0000 |
| 347674 | −0.92 | 0.91 | 0.16 | 1.80 | 0.31 | 1.60 | 160 | 0.43 | −0.61 | −0.76 | −0.68 | 6.244 | 4.0000 |
| 346418 | 0.17 | 1.64 | −0.26 | 1.42 | −2.35 | 0.66 | 164 | 0.84 | 1.26 | 0.66 | 1.83 | 10.969 | 4.0000 |
| 343414 | 0.22 | 1.52 | 0.02 | 1.79 | −1.45 | 1.07 | 161 | 0.76 | 0.83 | 0.76 | 1.16 | 7.180 | 4.0000 |
| 347642 | −0.79 | 0.91 | −0.78 | 1.16 | 0.42 | 1.72 | 163 | 0.25 | −0.60 | 0.98 | −0.31 | 6.597 | 3.9210 |
| 350008 | 0.34 | 1.84 | −0.21 | 1.34 | −0.86 | 0.90 | 163 | 0.68 | 0.60 | 0.08 | 0.62 | 6.458 | 3.9210 |
| 347715 | −1.51 | 0.66 | −0.16 | 1.77 | 0.18 | 1.59 | 158 | 0.18 | −0.84 | −0.60 | −1.17 | 7.512 | 3.9210 |
| 350575 | 0.00 | 0.00 | −1.42 | 0.46 | 0.06 | 1.63 | 163 | 0.02 | −0.03 | 48.33 | 1.35 | 9.925 | 3.8860 |
| 343818 | −0.96 | 0.49 | −0.53 | 1.22 | 0.28 | 1.76 | 141 | 0.23 | −0.62 | 0.31 | −0.52 | 7.036 | 3.8540 |
| 353087 | 0.00 | 0.00 | −1.27 | 1.04 | 0.17 | 1.62 | 162 | 0.07 | −0.09 | 15.94 | 1.09 | 7.262 | 3.8540 |
| 351586 | 0.13 | 1.63 | −1.23 | 0.95 | 0.00 | 0.00 | 163 | 0.94 | 0.07 | −19.92 | −1.08 | 7.160 | 3.8540 |
| 344851 | −2.29 | 0.78 | −0.61 | 0.97 | 0.13 | 1.61 | 161 | 0.11 | −1.21 | −0.39 | −1.57 | 9.959 | 3.8240 |
| 343536 | 0.39 | 1.73 | 0.05 | 1.59 | −0.84 | 1.13 | 155 | 0.57 | 0.61 | 0.45 | 0.66 | 5.945 | 3.8240 |
| 348212 | 0.00 | 0.00 | −1.24 | 0.80 | 0.13 | 1.64 | 162 | 0.05 | −0.07 | 20.08 | 1.10 | 7.794 | 3.7960 |
| 354084 | −1.34 | 0.98 | 0.16 | 1.90 | 0.16 | 1.48 | 163 | 0.26 | −0.75 | −1.00 | −1.11 | 6.725 | 3.7960 |
| 352521 | −0.99 | 0.99 | −0.42 | 1.38 | 0.38 | 1.74 | 155 | 0.25 | −0.69 | 0.17 | −0.63 | 6.618 | 3.7960 |
| 351679 | −0.90 | 0.85 | 0.33 | 1.78 | 0.07 | 1.63 | 162 | 0.36 | −0.48 | −1.54 | −0.69 | 6.325 | 3.7450 |
| 346888 | −1.79 | 0.72 | −1.11 | 1.14 | 0.18 | 1.61 | 162 | 0.08 | −0.98 | 0.31 | −0.73 | 7.776 | 3.7210 |
| 350825 | −0.77 | 1.18 | −0.41 | 1.31 | 0.45 | 1.74 | 164 | 0.34 | −0.61 | 0.41 | −0.53 | 6.109 | 3.7210 |
| 354170 | −0.01 | 1.60 | −1.58 | 0.37 | 0.00 | 0.00 | 159 | 0.98 | −0.01 | 315.00 | −1.52 | 11.132 | 3.6990 |
| 352674 | −2.35 | 0.66 | −0.24 | 1.53 | 0.14 | 1.60 | 164 | 0.14 | −1.25 | −0.69 | −1.87 | 10.404 | 3.6990 |
| 346417 | −1.55 | 0.81 | 0.18 | 2.11 | 0.07 | 1.38 | 161 | 0.21 | −0.81 | −1.14 | −1.35 | 7.128 | 3.6990 |
| 353461 | 0.16 | 1.65 | −1.22 | 1.06 | 0.00 | 0.00 | 157 | 0.93 | 0.08 | −16.25 | −1.03 | 6.927 | 3.6780 |
| 352384 | −1.16 | 1.20 | −0.02 | 1.50 | 0.26 | 1.66 | 162 | 0.27 | −0.71 | −0.61 | −0.91 | 6.459 | 3.6780 |
| 350574 | −0.90 | 1.71 | 0.56 | 1.68 | −0.06 | 1.38 | 164 | 0.35 | −0.42 | −2.48 | −0.72 | 6.107 | 3.6780 |
| 343824 | 0.10 | 1.54 | 0.19 | 1.73 | −1.30 | 1.11 | 161 | 0.70 | 0.70 | 1.13 | 1.02 | 6.795 | 3.6780 |
| 342814 | 0.07 | 1.60 | −1.52 | 0.85 | 0.00 | 0.00 | 162 | 0.97 | 0.04 | −44.43 | −1.41 | 7.848 | 3.6580 |
| 344028 | −1.70 | 0.59 | −0.88 | 1.39 | 0.17 | 1.60 | 164 | 0.09 | −0.93 | 0.12 | −0.84 | 7.391 | 3.6380 |
| 348372 | 0.00 | 0.00 | −0.87 | 1.28 | 0.22 | 1.63 | 158 | 0.11 | −0.11 | 8.91 | 0.66 | 5.816 | 3.6200 |
| 349762 | −0.68 | 1.25 | 0.04 | 1.30 | 0.50 | 1.91 | 161 | 0.49 | −0.59 | −0.22 | −0.59 | 5.682 | 3.6200 |
| 352680 | 0.32 | 1.65 | −0.41 | 1.35 | −1.20 | 1.28 | 162 | 0.78 | 0.76 | 0.04 | 0.78 | 6.457 | 3.6020 |
| 354324 | 0.50 | 1.75 | −0.18 | 1.59 | −0.66 | 1.11 | 163 | 0.58 | 0.58 | −0.17 | 0.56 | 5.787 | 3.5690 |
| 351608 | 0.54 | 1.61 | 0.00 | 1.63 | −0.65 | 1.43 | 163 | 0.52 | 0.59 | 0.09 | 0.60 | 5.592 | 3.5690 |
| 348758 | 0.15 | 1.62 | 0.33 | 1.67 | −1.11 | 1.33 | 156 | 0.71 | 0.63 | 1.29 | 0.96 | 6.104 | 3.5380 |
| 350947 | −1.30 | 0.53 | −0.09 | 1.67 | 0.16 | 1.64 | 160 | 0.21 | −0.73 | −0.66 | −1.01 | 7.550 | 3.5380 |
| 352361 | −1.16 | 1.09 | 0.24 | 1.81 | 0.03 | 1.42 | 164 | 0.34 | −0.59 | −1.35 | −0.86 | 6.611 | 3.4950 |
| 344319 | 0.26 | 1.67 | −0.04 | 1.49 | −1.05 | 1.20 | 164 | 0.72 | 0.66 | 0.54 | 0.81 | 6.104 | 3.4950 |
| 350281 | −1.89 | 0.52 | −0.76 | 1.50 | 0.24 | 1.58 | 164 | 0.13 | −1.06 | −0.06 | −1.11 | 9.726 | 3.4950 |
| 353897 | −0.76 | 1.26 | 0.41 | 1.64 | 0.03 | 1.67 | 161 | 0.45 | −0.40 | −1.96 | −0.47 | 5.576 | 3.4810 |
| 349269 | 0.77 | 1.76 | −0.16 | 1.65 | −0.47 | 1.21 | 164 | 0.47 | 0.62 | −0.50 | 0.64 | 5.753 | 3.4810 |
| 346008 | −0.84 | 1.14 | 0.35 | 1.62 | 0.01 | 1.71 | 163 | 0.42 | −0.42 | −1.80 | −0.55 | 5.801 | 3.4810 |
| 347206 | −0.97 | 1.03 | −0.49 | 1.07 | 0.38 | 1.81 | 164 | 0.25 | −0.68 | 0.29 | −0.58 | 6.697 | 3.4440 |
| 350424 | −0.84 | 0.93 | 0.29 | 1.46 | 0.11 | 1.87 | 163 | 0.38 | −0.47 | −1.38 | −0.63 | 5.854 | 3.4440 |
| 347075 | −0.70 | 1.56 | −0.02 | 1.49 | 0.57 | 1.69 | 164 | 0.48 | −0.64 | −0.07 | −0.64 | 5.636 | 3.4320 |
| 344554 | 0.10 | 1.60 | −1.65 | 0.98 | 0.00 | 0.00 | 164 | 0.96 | 0.05 | −34.00 | −1.52 | 7.658 | 3.4320 |
| 346154 | −1.39 | 0.62 | 0.81 | 1.83 | −0.16 | 1.51 | 161 | 0.15 | −0.61 | −2.58 | −1.74 | 9.011 | 3.4320 |
| 342909 | 0.17 | 1.61 | −1.06 | 1.15 | 0.00 | 0.00 | 164 | 0.93 | 0.09 | −13.47 | −0.89 | 6.186 | 3.4200 |
| 345719 | 0.38 | 1.71 | −0.10 | 1.67 | −0.75 | 0.93 | 164 | 0.62 | 0.56 | 0.15 | 0.59 | 5.819 | 3.4200 |
| 352287 | 0.16 | 1.60 | −0.78 | 0.83 | −1.63 | 0.90 | 156 | 0.89 | 0.89 | −0.05 | 0.86 | 7.408 | 3.4200 |
| 345309 | −0.78 | 1.26 | 0.31 | 1.72 | 0.41 | 1.56 | 161 | 0.55 | −0.59 | −0.83 | −0.55 | 5.644 | 3.3980 |
| 342572 | −1.24 | 1.34 | 0.37 | 1.88 | 0.02 | 1.44 | 163 | 0.26 | −0.63 | −1.56 | −1.10 | 6.388 | 3.3770 |
| 353697 | 0.30 | 1.50 | 0.40 | 1.55 | −0.72 | 1.44 | 163 | 0.45 | 0.51 | 1.20 | 0.45 | 5.493 | 3.3670 |
| 353169 | 0.17 | 1.59 | −1.25 | 1.04 | −2.20 | 0.82 | 157 | 0.93 | 1.19 | −0.20 | 0.98 | 8.359 | 3.3570 |
| 348656 | 0.07 | 1.64 | −1.24 | 0.70 | 0.00 | 0.00 | 163 | 0.97 | 0.04 | −36.43 | −1.15 | 7.400 | 3.3570 |
| 346905 | −0.88 | 1.31 | −0.11 | 1.29 | 0.35 | 1.78 | 159 | 0.36 | −0.61 | −0.25 | −0.66 | 5.848 | 3.3470 |
| 352931 | 0.00 | 0.00 | −1.51 | 1.10 | 0.11 | 1.60 | 163 | 0.04 | −0.06 | 28.45 | 1.39 | 6.882 | 3.3370 |
| 346584 | −1.56 | 0.30 | −0.99 | 1.06 | 0.17 | 1.64 | 164 | 0.09 | −0.86 | 0.34 | −0.62 | 9.550 | 3.3370 |
| 346520 | −1.25 | 0.56 | −0.45 | 1.39 | 0.23 | 1.76 | 149 | 0.18 | −0.74 | −0.08 | −0.78 | 7.402 | 3.3370 |
| 352000 | 0.13 | 1.59 | −1.68 | 1.16 | 0.00 | 0.00 | 163 | 0.95 | 0.07 | −26.85 | −1.51 | 7.127 | 3.3100 |
| 351213 | −1.09 | 1.30 | 0.22 | 1.60 | 0.16 | 1.61 | 164 | 0.30 | −0.62 | −1.10 | −0.89 | 5.898 | 3.3010 |
| 345544 | −1.07 | 1.12 | 0.14 | 1.63 | 0.15 | 1.62 | 154 | 0.38 | −0.61 | −0.98 | −0.75 | 5.857 | 3.2760 |
| 344969 | −0.65 | 1.20 | 0.35 | 1.89 | 0.57 | 1.46 | 164 | 0.60 | −0.61 | −0.64 | −0.53 | 5.620 | 3.2760 |
| 353680 | −1.03 | 1.11 | 0.17 | 1.44 | 0.19 | 1.74 | 161 | 0.29 | −0.61 | −0.97 | −0.86 | 5.880 | 3.2760 |
| 352213 | −0.16 | 1.09 | 0.56 | 1.86 | −0.54 | 1.40 | 163 | 0.41 | 0.19 | 4.79 | 0.03 | 5.586 | 3.2600 |
| 343542 | 0.13 | 1.66 | −0.30 | 1.36 | −1.65 | 0.71 | 163 | 0.87 | 0.89 | 0.52 | 1.23 | 7.729 | 3.2520 |
| 353584 | −0.19 | 1.63 | 0.40 | 1.66 | −0.85 | 0.60 | 161 | 0.73 | 0.33 | 2.79 | 0.76 | 6.699 | 3.2440 |
| 348323 | 0.09 | 1.62 | −1.37 | 0.86 | 0.00 | 0.00 | 164 | 0.96 | 0.04 | −31.44 | −1.26 | 7.089 | 3.2290 |
| 344636 | −1.84 | 0.62 | 0.33 | 1.98 | −0.02 | 1.50 | 164 | 0.13 | −0.91 | −1.38 | −1.84 | 8.215 | 3.2290 |
| 353436 | −1.27 | 0.87 | −0.29 | 1.85 | 0.19 | 1.47 | 142 | 0.23 | −0.73 | −0.34 | −0.87 | 5.921 | 3.2220 |
| 349668 | 0.45 | 1.78 | −0.03 | 1.48 | −0.66 | 1.33 | 163 | 0.56 | 0.56 | 0.14 | 0.56 | 5.354 | 3.2150 |
| 349644 | −1.09 | 1.30 | 0.22 | 1.60 | 0.16 | 1.61 | 164 | 0.30 | −0.62 | −1.10 | −0.89 | 5.898 | 3.2010 |
| 349469 | −0.53 | 1.17 | −0.62 | 1.07 | 0.46 | 1.84 | 163 | 0.32 | −0.49 | 1.18 | −0.29 | 5.622 | 3.2010 |
| 350456 | −1.09 | 1.30 | 0.18 | 1.60 | 0.14 | 1.61 | 162 | 0.31 | −0.61 | −1.07 | −0.87 | 5.719 | 3.1870 |
| 349388 | 0.09 | 1.50 | −0.16 | 1.96 | −2.44 | 0.42 | 164 | 0.88 | 1.26 | 0.80 | 2.04 | 10.054 | 3.1870 |
| 350400 | 0.18 | 1.63 | −1.00 | 1.22 | 0.00 | 0.00 | 163 | 0.91 | 0.09 | −12.11 | −0.81 | 5.853 | 3.1800 |
| 346040 | 0.13 | 1.68 | −0.19 | 1.48 | −1.37 | 0.43 | 163 | 0.84 | 0.75 | 0.57 | 1.04 | 7.903 | 3.1800 |
| 352386 | 0.14 | 1.62 | −1.18 | 1.13 | 0.00 | 0.00 | 163 | 0.94 | 0.07 | −17.86 | −1.04 | 6.280 | 3.1740 |
| 344286 | −1.13 | 1.15 | −0.05 | 1.12 | 0.19 | 1.76 | 162 | 0.22 | −0.66 | −0.64 | −0.90 | 6.125 | 3.1670 |
| 351811 | 0.17 | 1.63 | −1.00 | 1.22 | 0.00 | 0.00 | 164 | 0.91 | 0.09 | −12.76 | −0.81 | 5.805 | 3.1610 |
| 354030 | 0.00 | 0.00 | −0.92 | 0.99 | 0.14 | 1.65 | 164 | 0.07 | −0.07 | 14.14 | 0.78 | 5.895 | 3.1430 |
| 348202 | −0.86 | 1.03 | 0.26 | 1.91 | 0.12 | 1.44 | 162 | 0.40 | −0.49 | −1.29 | −0.62 | 5.571 | 3.1310 |
| 351492 | 0.14 | 1.64 | −1.01 | 1.11 | 0.00 | 0.00 | 161 | 0.93 | 0.07 | −15.43 | −0.86 | 5.807 | 3.1310 |
| 345154 | 0.13 | 1.61 | 0.17 | 1.57 | −1.09 | 1.30 | 161 | 0.70 | 0.61 | 1.07 | 0.87 | 5.627 | 3.1250 |
| 354499 | −0.44 | 1.17 | −0.03 | 1.85 | 0.81 | 1.58 | 161 | 0.60 | −0.62 | 0.34 | −0.67 | 5.530 | 3.1250 |
| 350377 | 0.33 | 1.63 | −0.03 | 1.80 | −0.78 | 1.00 | 163 | 0.62 | 0.56 | 0.35 | 0.60 | 5.447 | 3.1190 |
| 346166 | 0.57 | 1.69 | 0.00 | 1.50 | −0.69 | 1.60 | 160 | 0.53 | 0.63 | 0.10 | 0.63 | 5.467 | 3.0970 |
| 343943 | 0.38 | 1.72 | −0.45 | 1.32 | −1.00 | 1.14 | 163 | 0.74 | 0.69 | −0.20 | 0.62 | 6.234 | 3.0920 |
| 354350 | 0.23 | 1.71 | −0.29 | 1.31 | −1.46 | 1.03 | 164 | 0.80 | 0.84 | 0.38 | 1.04 | 7.110 | 3.0860 |
| 346701 | 0.08 | 1.64 | −1.08 | 0.77 | 0.00 | 0.00 | 164 | 0.96 | 0.04 | −28.00 | −0.99 | 6.511 | 3.0860 |
| 348725 | −0.82 | 0.86 | 0.28 | 1.49 | 0.08 | 1.86 | 159 | 0.35 | −0.45 | −1.44 | −0.64 | 5.681 | 3.0710 |
| 345797 | 0.08 | 1.65 | −1.05 | 0.76 | 0.00 | 0.00 | 163 | 0.96 | 0.04 | −27.25 | −0.96 | 6.419 | 3.0710 |
| 353046 | −0.76 | 1.40 | 0.23 | 1.35 | 0.36 | 1.81 | 158 | 0.42 | −0.56 | −0.77 | −0.63 | 5.330 | 3.0710 |
| 349071 | −0.43 | 1.44 | 0.06 | 1.68 | 1.08 | 1.47 | 163 | 0.65 | −0.76 | 0.35 | −0.83 | 6.069 | 3.0410 |
| 347100 | −0.23 | 1.15 | −0.34 | 1.64 | 0.93 | 1.96 | 164 | 0.59 | −0.58 | 1.19 | −0.70 | 5.369 | 3.0410 |
| 352332 | 0.00 | 0.00 | −1.44 | 0.96 | 0.12 | 1.61 | 163 | 0.05 | −0.06 | 25.00 | 1.30 | 6.918 | 3.0410 |
| 347705 | 0.42 | 1.69 | −0.03 | 1.72 | −0.65 | 0.95 | 164 | 0.55 | 0.54 | 0.16 | 0.54 | 5.364 | 3.0320 |
| 345203 | −0.32 | 1.63 | 0.97 | 1.97 | −0.19 | 1.03 | 158 | 0.61 | −0.07 | −18.85 | 0.20 | 5.391 | 3.0130 |
| 351244 | 0.00 | 0.00 | −1.02 | 0.91 | 0.08 | 1.65 | 154 | 0.06 | −0.04 | 26.50 | 0.90 | 5.927 | 3.0090 |
| 352119 | 0.12 | 1.65 | −1.02 | 1.02 | 0.00 | 0.00 | 161 | 0.93 | 0.06 | −18.00 | −0.88 | 5.946 | 3.0040 |
| 353547 | −0.96 | 1.58 | 0.04 | 1.56 | 0.35 | 1.55 | 151 | 0.34 | −0.66 | −0.53 | −0.76 | 5.449 | 3.0040 |
| 343827 | −1.28 | 1.28 | −0.40 | 1.47 | 0.29 | 1.62 | 163 | 0.21 | −0.79 | −0.12 | −0.84 | 6.209 | 3.0000 |
| 348058 | 0.02 | 1.51 | 0.25 | 1.86 | −1.13 | 0.93 | 164 | 0.75 | 0.57 | 1.40 | 0.98 | 6.196 | 2.9960 |
| 349306 | −0.96 | 1.25 | −0.08 | 1.79 | 0.31 | 1.49 | 164 | 0.32 | −0.64 | −0.39 | −0.72 | 5.713 | 2.9960 |
| 351077 | 0.44 | 1.80 | −0.21 | 1.47 | −0.64 | 1.21 | 163 | 0.61 | 0.54 | −0.20 | 0.52 | 5.247 | 2.9790 |
| 344033 | 0.78 | 1.77 | −0.58 | 1.47 | −0.02 | 1.59 | 150 | 0.33 | 0.40 | −2.40 | 0.72 | 5.378 | 2.9710 |
| 353944 | −0.50 | 1.42 | 0.35 | 1.71 | 0.00 | 0.00 | 153 | 0.69 | −0.25 | −2.40 | −0.02 | 4.739 | 2.9710 |
| 344673 | −0.98 | 0.56 | 0.18 | 0.97 | 0.00 | 1.78 | 164 | 0.15 | −0.49 | −1.37 | −0.96 | 6.711 | 2.9670 |
| 350425 | −0.80 | 0.90 | 0.24 | 1.49 | 0.08 | 1.86 | 164 | 0.37 | −0.44 | −1.36 | −0.60 | 5.437 | 2.9630 |
| 351471 | 0.00 | 0.00 | −1.56 | 1.20 | 0.12 | 1.60 | 162 | 0.04 | −0.06 | 27.00 | 1.44 | 6.450 | 2.9630 |
| 348726 | 0.08 | 1.86 | 0.24 | 1.49 | −0.80 | 0.90 | 164 | 0.63 | 0.44 | 1.36 | 0.60 | 5.437 | 2.9590 |
| 350827 | −1.27 | 1.08 | −1.55 | 1.05 | 0.21 | 1.60 | 161 | 0.10 | −0.74 | 1.38 | 0.08 | 6.293 | 2.9550 |
| 350404 | 0.10 | 1.59 | 0.07 | 1.62 | −1.61 | 1.04 | 164 | 0.74 | 0.86 | 0.96 | 1.25 | 7.066 | 2.9430 |
| 344814 | −1.40 | 0.69 | −0.25 | 1.56 | 0.17 | 1.65 | 163 | 0.18 | −0.78 | −0.46 | −1.02 | 7.271 | 2.9320 |
| 343861 | 0.12 | 1.64 | −1.07 | 1.14 | 0.00 | 0.00 | 162 | 0.94 | 0.06 | −18.83 | −0.92 | 5.663 | 2.9320 |
| 352920 | 0.08 | 1.86 | 0.24 | 1.49 | −0.80 | 0.90 | 164 | 0.63 | 0.44 | 1.36 | 0.60 | 5.437 | 2.9280 |
| 342874 | −1.91 | 1.19 | −0.70 | 1.30 | 0.27 | 1.61 | 161 | 0.14 | −1.09 | −0.11 | −1.18 | 7.127 | 2.9280 |
| 349580 | −1.69 | 0.22 | 0.58 | 1.44 | −0.06 | 1.63 | 164 | 0.08 | −0.81 | −1.79 | −2.05 | 10.309 | 2.9210 |
| 350161 | −0.58 | 1.25 | 0.00 | 1.51 | 0.51 | 1.91 | 162 | 0.51 | −0.54 | −0.06 | −0.54 | 5.031 | 2.9170 |
| 349442 | −0.72 | 1.15 | −0.24 | 1.40 | 0.38 | 1.80 | 162 | 0.34 | −0.55 | 0.13 | −0.53 | 5.413 | 2.9100 |
| 352177 | 0.00 | 0.00 | −0.91 | 0.87 | 0.13 | 1.71 | 145 | 0.08 | −0.07 | 15.00 | 0.76 | 5.777 | 2.8960 |
| 347915 | −0.68 | 1.55 | 0.16 | 1.60 | 0.41 | 1.54 | 163 | 0.49 | −0.55 | −0.54 | −0.55 | 5.089 | 2.8930 |
| 347760 | −0.20 | 1.12 | −1.28 | 0.94 | 0.16 | 1.64 | 147 | 0.17 | −0.18 | 7.00 | 0.64 | 6.096 | 2.8930 |
| 352426 | −1.04 | 1.16 | −0.19 | 1.41 | 0.26 | 1.71 | 163 | 0.25 | −0.65 | −0.31 | −0.75 | 5.864 | 2.8890 |
| 348518 | 0.31 | 1.56 | 0.00 | 1.80 | −0.76 | 1.11 | 161 | 0.61 | 0.54 | 0.42 | 0.58 | 5.237 | 2.8860 |
| 354395 | 0.23 | 1.49 | 0.19 | 1.84 | −0.86 | 1.22 | 164 | 0.62 | 0.55 | 0.93 | 0.66 | 5.208 | 2.8830 |
| 353918 | −1.04 | 1.16 | −0.19 | 1.41 | 0.24 | 1.71 | 164 | 0.25 | −0.64 | −0.33 | −0.75 | 5.799 | 2.8790 |
| 344998 | 0.91 | 1.36 | 0.17 | 1.71 | −0.52 | 1.42 | 146 | 0.37 | 0.72 | −0.03 | 0.72 | 5.851 | 2.8730 |
| 353335 | −0.96 | 1.05 | −0.38 | 1.28 | 0.33 | 1.76 | 161 | 0.24 | −0.65 | 0.10 | −0.61 | 5.986 | 2.8700 |
| 352427 | 0.06 | 1.53 | 0.13 | 2.01 | −1.69 | 0.49 | 159 | 0.87 | 0.88 | 1.08 | 1.57 | 7.113 | 2.8660 |
| 345016 | −0.52 | 1.30 | 0.01 | 1.48 | 0.73 | 2.04 | 163 | 0.57 | −0.62 | 0.15 | −0.64 | 5.155 | 2.8630 |
| 348760 | −1.08 | 1.33 | 0.17 | 1.61 | 0.16 | 1.60 | 164 | 0.30 | −0.62 | −1.02 | −0.87 | 5.516 | 2.8600 |
| 343384 | 0.18 | 1.60 | −0.36 | 1.48 | −1.85 | 1.04 | 162 | 0.83 | 1.02 | 0.47 | 1.33 | 7.277 | 2.8600 |
| 344020 | 0.00 | 0.00 | −1.76 | 1.05 | 0.09 | 1.61 | 160 | 0.04 | −0.04 | 40.11 | 1.61 | 6.986 | 2.8510 |
| 348445 | −1.10 | 0.52 | −0.48 | 1.23 | 0.25 | 1.75 | 163 | 0.19 | −0.68 | 0.08 | −0.64 | 7.429 | 2.8510 |
| 353479 | −0.65 | 1.26 | 0.18 | 1.60 | 0.56 | 1.90 | 163 | 0.56 | −0.60 | −0.37 | −0.58 | 5.114 | 2.8480 |
| 344738 | 0.00 | 0.00 | −1.76 | 1.05 | 0.09 | 1.61 | 160 | 0.04 | −0.04 | 40.11 | 1.61 | 6.981 | 2.8420 |
| 350818 | 0.43 | 1.89 | −0.09 | 1.29 | −0.61 | 1.35 | 161 | 0.56 | 0.52 | 0.00 | 0.52 | 5.032 | 2.8300 |
| 347635 | 0.34 | 1.50 | −0.05 | 1.64 | −0.86 | 1.57 | 163 | 0.63 | 0.60 | 0.35 | 0.66 | 5.267 | 2.8270 |
| 350386 | −0.62 | 1.38 | −0.12 | 1.32 | 0.65 | 2.10 | 163 | 0.47 | −0.64 | 0.21 | −0.63 | 5.193 | 2.8180 |
| 348421 | −1.17 | 0.93 | −0.04 | 1.62 | 0.16 | 1.61 | 145 | 0.21 | −0.66 | −0.70 | −0.93 | 5.518 | 2.8180 |
| 353211 | 0.55 | 1.73 | −0.68 | 1.30 | 0.16 | 1.73 | 148 | 0.37 | 0.20 | −5.31 | 0.47 | 5.165 | 2.8150 |
| 353020 | −0.67 | 1.36 | 0.27 | 1.75 | 0.42 | 1.59 | 159 | 0.53 | −0.55 | −0.72 | −0.52 | 4.989 | 2.8120 |
| 344073 | −1.20 | 1.42 | −0.10 | 1.49 | 0.22 | 1.62 | 162 | 0.23 | −0.71 | −0.55 | −0.92 | 5.629 | 2.8100 |
| 346122 | 0.05 | 1.62 | −1.75 | 0.82 | 0.00 | 0.00 | 160 | 0.97 | 0.03 | −71.00 | −1.65 | 7.558 | 2.7990 |
| 351538 | 0.18 | 1.63 | −0.86 | 1.25 | −1.69 | 0.67 | 158 | 0.88 | 0.93 | −0.11 | 0.85 | 7.905 | 2.7990 |
| 344342 | 0.08 | 1.86 | 0.24 | 1.49 | −0.79 | 0.91 | 163 | 0.64 | 0.43 | 1.37 | 0.60 | 5.299 | 2.7930 |
| 351772 | −0.80 | 1.79 | 0.32 | 1.46 | 0.21 | 1.47 | 164 | 0.47 | −0.51 | −1.22 | −0.54 | 5.039 | 2.7900 |
| 354108 | 0.00 | 0.00 | −1.75 | 0.82 | 0.06 | 1.61 | 164 | 0.03 | −0.03 | 59.33 | 1.65 | 7.596 | 2.7880 |
| 354008 | −1.31 | 1.37 | −0.45 | 1.40 | 0.24 | 1.61 | 164 | 0.17 | −0.78 | −0.11 | −0.83 | 5.726 | 2.7720 |
| 349347 | 0.16 | 1.65 | −0.71 | 1.31 | −1.58 | 0.07 | 163 | 0.90 | 0.87 | 0.00 | 0.87 | 10.331 | 2.7700 |
| 352088 | 0.08 | 1.61 | −1.50 | 0.91 | 0.00 | 0.00 | 163 | 0.97 | 0.04 | −38.50 | −1.41 | 6.785 | 2.7700 |
| 348232 | 0.00 | 0.00 | −1.03 | 1.02 | 0.11 | 1.64 | 164 | 0.06 | −0.06 | 19.73 | 0.90 | 5.696 | 2.7670 |
| 346676 | 0.00 | 0.00 | −0.79 | 1.27 | 0.16 | 1.62 | 162 | 0.10 | −0.08 | 10.88 | 0.61 | 4.958 | 2.7640 |
| 348353 | 0.08 | 1.60 | −1.62 | 1.00 | 0.00 | 0.00 | 164 | 0.97 | 0.04 | −41.50 | −1.53 | 6.728 | 2.7640 |
| 348231 | 0.00 | 0.00 | −1.03 | 1.02 | 0.11 | 1.64 | 164 | 0.06 | −0.06 | 19.73 | 0.90 | 5.696 | 2.7640 |
| 352928 | −0.10 | 1.54 | 0.51 | 1.74 | −0.52 | 1.39 | 163 | 0.50 | 0.21 | 3.90 | 0.20 | 4.879 | 2.7540 |
| 352149 | 0.25 | 1.68 | 0.17 | 1.63 | −0.80 | 1.30 | 162 | 0.58 | 0.53 | 0.85 | 0.59 | 5.005 | 2.7520 |
| 349571 | 0.65 | 2.10 | −0.12 | 1.32 | −0.57 | 1.38 | 164 | 0.52 | 0.61 | −0.26 | 0.60 | 5.037 | 2.7520 |
| 354476 | 0.75 | 1.36 | 0.14 | 1.85 | −0.38 | 1.22 | 130 | 0.47 | 0.56 | −0.08 | 0.57 | 4.992 | 2.7450 |
| 342939 | 0.29 | 1.78 | −0.21 | 1.42 | −0.89 | 0.97 | 161 | 0.71 | 0.59 | 0.15 | 0.63 | 5.706 | 2.7420 |
| 343882 | 0.00 | 0.00 | −1.89 | 0.42 | 0.06 | 1.61 | 161 | 0.01 | −0.03 | 64.00 | 1.84 | 11.084 | 2.7400 |
| 349265 | −0.40 | 1.21 | −0.15 | 1.59 | 0.87 | 1.96 | 163 | 0.56 | −0.64 | 0.61 | −0.68 | 5.164 | 2.7380 |
| 350229 | 0.38 | 1.59 | −0.01 | 1.74 | −0.67 | 1.19 | 164 | 0.56 | 0.53 | 0.26 | 0.54 | 5.059 | 2.7380 |
| 346006 | 0.00 | 0.00 | −1.75 | 0.82 | 0.06 | 1.63 | 160 | 0.03 | −0.03 | 59.33 | 1.65 | 7.566 | 2.7280 |
| 348673 | 0.00 | 0.00 | −0.76 | 0.61 | 0.07 | 1.68 | 164 | 0.05 | −0.04 | 22.71 | 0.67 | 5.661 | 2.7240 |
| 344070 | −0.47 | 1.15 | −0.05 | 1.81 | 0.60 | 1.51 | 161 | 0.52 | −0.53 | 0.21 | −0.54 | 5.080 | 2.7210 |
| 347434 | 0.57 | 1.79 | −0.20 | 1.71 | −0.44 | 0.92 | 164 | 0.53 | 0.51 | −0.52 | 0.49 | 4.964 | 2.7140 |
| 347385 | −0.66 | 1.27 | −0.09 | 1.62 | 0.39 | 1.69 | 159 | 0.43 | −0.53 | −0.09 | −0.53 | 4.987 | 2.7120 |
| 354346 | 0.06 | 1.43 | −0.50 | 1.37 | 1.09 | 2.21 | 164 | 0.65 | −0.52 | 2.09 | −0.84 | 5.406 | 2.7080 |
| 348636 | 0.05 | 1.63 | −1.51 | 0.58 | 0.00 | 0.00 | 164 | 0.98 | 0.03 | −61.40 | −1.44 | 8.176 | 2.7030 |
| 342599 | 0.00 | 0.00 | −1.77 | 1.26 | 0.12 | 1.58 | 163 | 0.04 | −0.06 | 30.50 | 1.61 | 6.371 | 2.7030 |
| 346674 | 0.38 | 1.59 | −0.01 | 1.74 | −0.67 | 1.19 | 164 | 0.56 | 0.53 | 0.26 | 0.54 | 5.059 | 2.7010 |
| 353784 | 0.41 | 1.65 | −0.08 | 1.45 | −0.63 | 1.35 | 148 | 0.53 | 0.52 | 0.06 | 0.52 | 4.895 | 2.6930 |
| 344298 | −1.07 | 0.99 | −0.32 | 1.88 | 0.29 | 1.62 | 154 | 0.23 | −0.68 | −0.10 | −0.72 | 5.359 | 2.6900 |
| 350943 | 0.29 | 1.58 | −0.32 | 1.62 | −0.83 | 1.21 | 156 | 0.69 | 0.56 | −0.09 | 0.54 | 5.087 | 2.6820 |
| 350677 | −0.65 | 1.42 | 0.39 | 1.74 | 0.07 | 1.33 | 161 | 0.57 | −0.36 | −1.89 | −0.27 | 5.007 | 2.6720 |
| 348383 | −0.58 | 1.45 | −0.07 | 1.49 | 0.52 | 1.78 | 159 | 0.44 | −0.55 | 0.07 | −0.55 | 4.922 | 2.6720 |
| 353070 | −1.49 | 0.86 | −0.02 | 1.89 | 0.20 | 1.49 | 153 | 0.22 | −0.84 | −0.74 | −1.20 | 6.733 | 2.6680 |
| 351278 | 2.62 | 0.41 | 0.00 | 0.00 | −0.11 | 1.55 | 150 | 0.02 | 1.36 | −0.92 | 2.56 | 14.297 | 2.6580 |
| 344428 | 0.34 | 1.67 | −0.24 | 1.45 | −0.90 | 1.43 | 163 | 0.71 | 0.62 | 0.06 | 0.64 | 5.327 | 2.6580 |
| 354036 | −0.94 | 0.81 | −0.06 | 1.70 | 0.18 | 1.64 | 164 | 0.27 | −0.56 | −0.57 | −0.71 | 5.605 | 2.6540 |
| 353891 | 0.00 | 0.00 | −0.77 | 0.63 | 0.07 | 1.68 | 163 | 0.05 | −0.04 | 23.00 | 0.69 | 5.536 | 2.6440 |
| 348151 | 0.16 | 1.55 | −0.04 | 1.73 | −1.29 | 1.12 | 164 | 0.77 | 0.72 | 0.72 | 1.00 | 5.936 | 2.6380 |
| 350535 | −1.52 | 0.89 | −0.32 | 1.91 | 0.16 | 1.54 | 163 | 0.14 | −0.84 | −0.43 | −1.10 | 5.874 | 2.6380 |
| 347681 | −1.60 | 1.06 | −0.62 | 1.10 | 0.17 | 1.67 | 159 | 0.11 | −0.89 | −0.11 | −0.96 | 6.305 | 2.6350 |
| 348040 | −0.22 | 1.10 | −0.34 | 1.68 | 0.74 | 1.91 | 164 | 0.55 | −0.48 | 1.25 | −0.54 | 4.852 | 2.6330 |
| 348152 | −1.29 | 1.12 | −0.04 | 1.73 | 0.16 | 1.55 | 164 | 0.23 | −0.72 | −0.72 | −1.00 | 5.936 | 2.6290 |
| 346369 | −1.05 | 1.49 | 0.26 | 1.76 | 0.05 | 1.44 | 164 | 0.33 | −0.55 | −1.38 | −0.81 | 5.385 | 2.6270 |
| 350228 | −0.67 | 1.19 | −0.01 | 1.74 | 0.36 | 1.60 | 163 | 0.44 | −0.52 | −0.28 | −0.53 | 4.966 | 2.6250 |
| 344787 | 0.11 | 1.55 | 0.04 | 1.82 | −1.21 | 0.86 | 164 | 0.78 | 0.66 | 0.89 | 0.99 | 5.835 | 2.6200 |
| 346313 | 0.16 | 1.55 | −0.04 | 1.73 | −1.29 | 1.12 | 164 | 0.77 | 0.72 | 0.72 | 1.00 | 5.936 | 2.6160 |
| 349144 | −1.44 | 0.52 | −0.43 | 1.37 | 0.16 | 1.67 | 164 | 0.14 | −0.80 | −0.26 | −0.95 | 7.690 | 2.6130 |
| 347188 | 0.17 | 1.56 | −0.04 | 1.73 | −1.29 | 1.12 | 163 | 0.76 | 0.73 | 0.71 | 1.00 | 5.960 | 2.6090 |
| 347933 | 0.00 | 0.00 | −0.96 | 0.84 | 0.09 | 1.69 | 155 | 0.05 | −0.04 | 22.33 | 0.87 | 5.620 | 2.6090 |
| 345764 | −0.68 | 1.37 | 0.17 | 1.64 | 0.44 | 1.67 | 164 | 0.54 | −0.56 | −0.52 | −0.54 | 4.938 | 2.6060 |
| 348039 | 0.74 | 1.91 | −0.34 | 1.68 | −0.22 | 1.10 | 163 | 0.45 | 0.48 | −1.25 | 0.54 | 4.842 | 2.6040 |
| 344576 | −1.35 | 1.17 | 0.01 | 1.69 | 0.16 | 1.54 | 164 | 0.28 | −0.76 | −0.80 | −1.02 | 6.074 | 2.6020 |
| 344501 | −0.77 | 1.05 | 0.02 | 1.67 | 0.26 | 1.70 | 161 | 0.36 | −0.52 | −0.53 | −0.59 | 5.028 | 2.5950 |
| 344268 | 0.79 | 1.85 | 0.03 | 1.54 | −0.46 | 1.46 | 164 | 0.41 | 0.62 | −0.22 | 0.65 | 5.045 | 2.5900 |
| 352564 | 0.39 | 1.79 | −0.18 | 1.37 | −0.71 | 1.43 | 163 | 0.63 | 0.55 | −0.04 | 0.54 | 4.997 | 2.5880 |
| 353929 | −0.56 | 1.10 | −0.11 | 1.87 | 0.42 | 1.35 | 156 | 0.46 | −0.49 | 0.08 | −0.49 | 4.884 | 2.5870 |
| 352051 | 0.35 | 1.68 | −0.18 | 1.69 | −0.87 | 1.12 | 153 | 0.72 | 0.61 | 0.13 | 0.65 | 5.055 | 2.5850 |
| 347050 | −0.90 | 0.97 | 0.20 | 1.90 | 0.09 | 1.49 | 159 | 0.32 | −0.49 | −1.22 | −0.71 | 5.247 | 2.5850 |
| 351998 | −0.45 | 1.56 | 0.02 | 1.50 | 0.70 | 1.77 | 163 | 0.59 | −0.57 | 0.18 | −0.59 | 4.899 | 2.5830 |
| 343273 | −0.30 | 1.49 | 0.54 | 1.73 | 0.00 | 0.00 | 162 | 0.82 | −0.15 | −4.60 | 0.30 | 4.364 | 2.5830 |
| 346192 | 0.00 | 0.00 | −1.65 | 1.20 | 0.08 | 1.59 | 162 | 0.03 | −0.04 | 42.25 | 1.55 | 6.088 | 2.5820 |
| 345765 | −0.68 | 1.37 | 0.17 | 1.64 | 0.44 | 1.67 | 164 | 0.54 | −0.56 | −0.52 | −0.54 | 4.938 | 2.5780 |
| 347866 | −1.41 | 0.96 | −0.92 | 1.28 | 0.21 | 1.62 | 164 | 0.11 | −0.81 | 0.40 | −0.56 | 6.091 | 2.5750 |
| 344061 | 0.12 | 1.65 | −0.91 | 1.07 | 0.00 | 0.00 | 163 | 0.94 | 0.06 | −16.17 | −0.79 | 5.213 | 2.5650 |
| 344916 | 0.00 | 0.00 | −0.83 | 0.92 | 0.11 | 1.67 | 162 | 0.07 | −0.06 | 16.09 | 0.70 | 5.203 | 2.5640 |
| 348149 | 0.24 | 1.63 | −0.83 | 1.35 | −1.15 | 0.36 | 163 | 0.87 | 0.69 | −0.54 | 0.41 | 7.386 | 2.5640 |
| 349197 | −0.80 | 0.94 | −0.18 | 1.63 | 0.29 | 1.70 | 158 | 0.28 | −0.55 | −0.14 | −0.58 | 5.240 | 2.5620 |
| 343085 | 0.00 | 0.00 | −1.45 | 1.03 | 0.08 | 1.61 | 164 | 0.04 | −0.04 | 37.25 | 1.34 | 6.188 | 2.5620 |
| 352342 | 0.38 | 1.74 | −0.35 | 1.36 | −0.66 | 1.14 | 150 | 0.63 | 0.52 | −0.40 | 0.46 | 5.043 | 2.5620 |
| 345043 | 0.66 | 1.54 | 0.28 | 1.59 | −0.49 | 1.56 | 163 | 0.35 | 0.57 | 0.34 | 0.52 | 5.025 | 2.5580 |
| 353221 | −0.78 | 1.34 | −0.30 | 1.60 | 0.45 | 1.63 | 138 | 0.31 | −0.61 | 0.22 | −0.56 | 5.030 | 2.5540 |
| 346914 | 0.00 | 0.00 | −0.78 | 1.33 | 0.17 | 1.65 | 162 | 0.10 | −0.09 | 10.18 | 0.60 | 4.694 | 2.5540 |
| 347684 | 0.66 | 1.49 | 0.20 | 1.60 | −0.46 | 1.58 | 159 | 0.36 | 0.56 | 0.18 | 0.53 | 4.936 | 2.5530 |
| 352986 | −0.55 | 0.95 | 0.80 | 1.82 | −0.20 | 1.57 | 158 | 0.21 | −0.18 | −6.71 | −0.87 | 5.550 | 2.5510 |
| 346380 | 0.00 | 0.00 | −1.39 | 1.20 | 0.10 | 1.62 | 161 | 0.04 | −0.05 | 28.80 | 1.28 | 5.708 | 2.5420 |
| 350385 | −0.95 | 0.80 | −0.61 | 1.23 | 0.30 | 1.73 | 162 | 0.19 | −0.62 | 0.46 | −0.45 | 6.085 | 2.5390 |
| 343966 | 0.01 | 1.67 | 0.41 | 1.76 | −0.69 | 1.22 | 146 | 0.62 | 0.35 | 2.14 | 0.53 | 4.855 | 2.5390 |
| 346001 | 0.12 | 1.62 | −1.07 | 1.24 | 0.00 | 0.00 | 164 | 0.94 | 0.06 | −18.83 | −0.93 | 5.215 | 2.5380 |
| 349814 | 0.21 | 1.63 | −0.03 | 1.68 | −0.91 | 0.96 | 164 | 0.68 | 0.56 | 0.57 | 0.67 | 5.459 | 2.5360 |
| 349594 | 0.31 | 1.71 | −0.16 | 1.46 | −0.81 | 1.36 | 163 | 0.68 | 0.56 | 0.16 | 0.59 | 5.066 | 2.5360 |
| 351373 | 0.21 | 1.63 | −0.03 | 1.68 | −0.91 | 0.96 | 164 | 0.68 | 0.56 | 0.57 | 0.67 | 5.459 | 2.5320 |
| 351612 | 0.00 | 0.00 | −1.32 | 1.11 | 0.09 | 1.61 | 164 | 0.04 | −0.04 | 30.33 | 1.22 | 5.741 | 2.5230 |
| 349970 | 0.00 | 0.00 | −0.91 | 1.20 | 0.11 | 1.65 | 161 | 0.07 | −0.06 | 17.55 | 0.78 | 4.923 | 2.5200 |
| 349822 | 0.25 | 1.80 | −0.36 | 1.24 | −1.00 | 0.78 | 162 | 0.78 | 0.62 | 0.02 | 0.63 | 6.187 | 2.5190 |
| 345005 | 0.10 | 1.69 | −0.74 | 0.80 | 0.00 | 0.00 | 162 | 0.94 | 0.05 | −15.80 | −0.64 | 5.119 | 2.5160 |
| 352695 | 0.12 | 1.59 | −1.57 | 1.30 | 0.00 | 0.00 | 163 | 0.95 | 0.06 | −27.17 | −1.42 | 5.782 | 2.5140 |
| 354175 | −0.84 | 0.87 | 0.19 | 1.74 | 0.13 | 1.66 | 149 | 0.41 | −0.48 | −1.12 | −0.58 | 5.014 | 2.5130 |
| 347884 | −0.51 | 1.35 | 0.24 | 1.51 | 0.59 | 1.92 | 164 | 0.60 | −0.55 | −0.36 | −0.51 | 4.768 | 2.5070 |
| 348693 | 0.11 | 1.59 | −1.73 | 1.20 | 0.00 | 0.00 | 162 | 0.97 | 0.06 | −32.45 | −1.61 | 6.214 | 2.5020 |
| 346314 | 0.15 | 1.66 | −0.73 | 1.20 | 0.00 | 0.00 | 163 | 0.90 | 0.07 | −10.73 | −0.57 | 4.679 | 2.4980 |
| 349256 | 0.47 | 1.50 | −0.03 | 1.76 | −0.54 | 1.44 | 157 | 0.50 | 0.51 | 0.01 | 0.51 | 4.681 | 2.4960 |
| 343858 | 0.21 | 1.44 | 0.36 | 1.56 | −0.62 | 1.51 | 158 | 0.46 | 0.41 | 1.36 | 0.37 | 4.708 | 2.4960 |
| 349547 | 0.00 | 0.00 | −1.43 | 0.74 | 0.07 | 1.63 | 163 | 0.03 | −0.04 | 41.86 | 1.33 | 6.861 | 2.4950 |
| 347424 | 0.48 | 1.78 | −0.10 | 1.48 | −0.58 | 1.45 | 163 | 0.56 | 0.53 | −0.09 | 0.52 | 4.816 | 2.4850 |
| 350557 | 0.13 | 1.65 | −0.26 | 1.47 | −1.65 | 0.81 | 164 | 0.86 | 0.89 | 0.56 | 1.25 | 6.973 | 2.4840 |
| 352232 | −0.81 | 1.34 | 0.27 | 1.63 | 0.18 | 1.64 | 160 | 0.35 | −0.49 | −1.18 | −0.67 | 4.801 | 2.4790 |
| 343112 | −1.44 | 1.22 | −0.49 | 1.57 | 0.19 | 1.60 | 162 | 0.13 | −0.81 | −0.17 | −0.91 | 5.415 | 2.4790 |
| 353148 | −0.96 | 1.59 | 0.00 | 1.52 | 0.26 | 1.62 | 164 | 0.34 | −0.61 | −0.57 | −0.73 | 5.060 | 2.4760 |
| 352360 | 0.00 | 0.00 | −1.02 | 0.69 | 0.05 | 1.65 | 163 | 0.03 | −0.03 | 41.80 | 0.96 | 5.964 | 2.4750 |
| 353346 | 0.15 | 1.72 | −0.84 | 0.99 | 0.00 | 0.00 | 152 | 0.93 | 0.07 | −12.20 | −0.71 | 5.046 | 2.4720 |
| 353923 | −0.64 | 1.23 | −0.03 | 1.66 | 0.38 | 1.68 | 164 | 0.44 | −0.51 | −0.20 | −0.52 | 4.843 | 2.4670 |
| 343386 | −1.38 | 0.95 | −0.63 | 1.43 | 0.17 | 1.62 | 164 | 0.12 | −0.77 | 0.03 | −0.76 | 5.759 | 2.4570 |
| 347533 | −0.35 | 1.08 | 0.55 | 2.03 | −0.40 | 1.31 | 161 | 0.49 | 0.03 | 37.00 | 0.00 | 4.651 | 2.4530 |
| 343173 | −0.46 | 1.29 | 0.54 | 1.90 | −0.31 | 1.19 | 162 | 0.61 | −0.08 | −12.33 | 0.12 | 4.870 | 2.4530 |
| 348065 | 0.20 | 1.68 | −1.29 | 1.13 | −0.48 | 0.72 | 150 | 0.89 | 0.34 | −3.38 | −0.56 | 6.239 | 2.4530 |
| 349672 | 0.00 | 0.00 | −0.89 | 0.86 | 0.07 | 1.66 | 163 | 0.05 | −0.04 | 26.43 | 0.80 | 5.209 | 2.4520 |
| 344596 | −0.35 | 1.62 | 0.67 | 1.72 | −0.18 | 1.21 | 159 | 0.63 | −0.08 | −11.00 | 0.15 | 4.785 | 2.4500 |
| 353126 | −0.01 | 1.35 | 0.38 | 1.87 | −0.97 | 1.67 | 164 | 0.70 | 0.48 | 1.81 | 0.84 | 5.047 | 2.4490 |
| 349614 | −1.13 | 0.15 | −1.30 | 0.91 | 0.09 | 1.58 | 162 | 0.06 | −0.61 | 1.28 | 0.07 | 8.270 | 2.4490 |
| 348104 | −1.37 | 0.40 | 0.11 | 1.71 | −0.01 | 1.60 | 164 | 0.18 | −0.68 | −1.18 | −1.19 | 7.549 | 2.4350 |
| 352804 | −0.74 | 0.94 | −0.36 | 1.01 | 0.22 | 1.81 | 163 | 0.22 | −0.48 | 0.21 | −0.42 | 5.126 | 2.4340 |
| 354536 | −1.03 | 1.02 | 0.34 | 1.71 | 0.00 | 1.60 | 159 | 0.23 | −0.52 | −1.66 | −0.97 | 5.706 | 2.4340 |
| 349305 | −0.48 | 0.88 | 0.55 | 1.83 | −0.33 | 1.49 | 163 | 0.31 | −0.07 | −12.73 | −0.43 | 5.182 | 2.4330 |
| 345430 | −1.14 | 0.67 | −0.40 | 1.24 | 0.22 | 1.75 | 164 | 0.19 | −0.68 | −0.09 | −0.72 | 6.632 | 2.4330 |
| 351977 | 0.11 | 1.71 | 0.00 | 1.14 | −1.19 | 0.98 | 138 | 0.85 | 0.65 | 0.83 | 1.03 | 5.224 | 2.4320 |
| 344874 | −0.76 | 0.85 | −0.17 | 1.76 | 0.27 | 1.60 | 164 | 0.30 | −0.52 | −0.15 | −0.54 | 5.186 | 2.4260 |
| 354494 | −1.06 | 1.05 | 0.23 | 1.91 | 0.10 | 1.59 | 151 | 0.25 | −0.58 | −1.22 | −0.94 | 5.209 | 2.4200 |
| 351995 | 0.60 | 1.84 | 0.09 | 1.65 | −0.51 | 1.29 | 164 | 0.43 | 0.55 | 0.08 | 0.55 | 4.722 | 2.4190 |
| 351507 | 0.05 | 1.73 | 0.29 | 1.77 | −0.64 | 0.87 | 163 | 0.56 | 0.35 | 1.70 | 0.41 | 4.668 | 2.4160 |
| 352888 | −0.67 | 1.46 | 0.33 | 1.66 | 0.10 | 1.59 | 164 | 0.42 | −0.39 | −1.60 | −0.48 | 4.636 | 2.4090 |
| 350881 | −0.15 | 1.52 | 0.18 | 1.76 | 2.86 | 1.14 | 164 | 0.89 | −1.50 | 0.78 | −2.41 | 7.760 | 2.4080 |
| 351110 | −1.77 | 0.56 | −0.71 | 1.80 | 0.12 | 1.52 | 159 | 0.10 | −0.95 | −0.12 | −1.04 | 6.736 | 2.4070 |
| 348166 | −0.43 | 1.16 | −0.14 | 1.62 | 0.55 | 1.89 | 164 | 0.51 | −0.49 | 0.41 | −0.49 | 4.541 | 2.4070 |
| 351834 | 0.07 | 1.22 | 0.34 | 1.94 | −0.62 | 1.39 | 163 | 0.52 | 0.34 | 1.78 | 0.36 | 4.619 | 2.4030 |
| 349618 | −1.14 | 0.62 | 0.32 | 1.86 | −0.03 | 1.58 | 162 | 0.15 | −0.55 | −1.63 | −1.19 | 6.024 | 2.4030 |
| 349013 | 0.23 | 1.80 | −0.23 | 1.41 | −0.75 | 0.68 | 164 | 0.74 | 0.49 | 0.06 | 0.50 | 5.385 | 2.3950 |
| 354228 | 0.20 | 1.41 | 0.41 | 1.55 | −0.82 | 1.83 | 151 | 0.66 | 0.51 | 1.41 | 0.73 | 4.819 | 2.3950 |
| 344165 | −0.78 | 1.03 | −0.12 | 1.65 | 0.22 | 1.63 | 156 | 0.35 | −0.50 | −0.32 | −0.55 | 4.860 | 2.3890 |
| 349942 | 0.05 | 1.65 | −0.90 | 0.54 | 0.00 | 0.00 | 164 | 0.96 | 0.03 | −37.00 | −0.83 | 5.999 | 2.3880 |
| 351869 | −0.86 | 1.19 | −0.10 | 1.43 | 0.26 | 1.81 | 162 | 0.33 | −0.56 | −0.36 | −0.63 | 5.093 | 2.3830 |
| 348888 | 0.22 | 1.70 | −0.03 | 1.57 | −1.17 | 1.09 | 164 | 0.65 | 0.69 | 0.64 | 0.83 | 5.799 | 2.3830 |
| 348462 | 0.26 | 1.70 | −0.36 | 1.39 | −1.00 | 1.12 | 162 | 0.78 | 0.63 | 0.02 | 0.64 | 5.407 | 2.3830 |
| 349735 | −1.08 | 1.00 | 0.81 | 1.86 | −0.18 | 1.51 | 162 | 0.15 | −0.45 | −3.20 | −1.46 | 6.360 | 2.3820 |
| 347806 | 0.00 | 0.00 | −0.96 | 1.15 | 0.10 | 1.64 | 164 | 0.05 | −0.05 | 20.20 | 0.85 | 4.945 | 2.3820 |
| 345359 | −0.48 | 1.11 | −0.18 | 1.31 | 0.48 | 2.06 | 164 | 0.45 | −0.48 | 0.38 | −0.46 | 4.643 | 2.3730 |
| 343979 | −0.86 | 0.69 | −0.29 | 1.56 | 0.18 | 1.68 | 162 | 0.22 | −0.52 | −0.10 | −0.55 | 5.262 | 2.3730 |
| 347440 | −0.40 | 1.15 | 0.47 | 1.75 | −0.47 | 1.52 | 164 | 0.45 | 0.03 | 25.86 | −0.05 | 4.694 | 2.3690 |
| 353222 | 0.47 | 1.62 | −0.07 | 1.74 | −0.48 | 1.18 | 159 | 0.48 | 0.47 | −0.14 | 0.48 | 4.527 | 2.3650 |
| 349717 | −0.53 | 1.17 | 0.23 | 1.64 | 0.52 | 1.95 | 162 | 0.57 | −0.53 | −0.45 | −0.49 | 4.564 | 2.3610 |
| 350617 | −1.06 | 1.06 | −0.17 | 1.68 | 0.21 | 1.60 | 163 | 0.25 | −0.64 | −0.40 | −0.76 | 5.479 | 2.3580 |
| 345930 | −0.46 | 1.46 | 0.01 | 1.55 | 0.79 | 1.88 | 161 | 0.60 | −0.62 | 0.25 | −0.66 | 4.866 | 2.3570 |
| 353905 | −1.98 | 0.50 | −0.38 | 1.84 | 0.07 | 1.55 | 161 | 0.09 | −1.02 | −0.56 | −1.50 | 7.236 | 2.3550 |
| 345048 | 0.08 | 1.63 | −1.17 | 0.97 | 0.00 | 0.00 | 163 | 0.95 | 0.04 | −30.25 | −1.06 | 5.481 | 2.3540 |
| 353573 | −0.68 | 1.36 | −0.24 | 1.43 | 0.38 | 1.75 | 163 | 0.34 | −0.53 | 0.17 | −0.50 | 4.836 | 2.3540 |
| 349673 | 0.00 | 0.00 | −0.80 | 0.86 | 0.07 | 1.67 | 164 | 0.05 | −0.04 | 23.86 | 0.71 | 4.979 | 2.3500 |
| 346157 | 2.73 | 1.17 | −0.28 | 1.24 | 0.00 | 1.71 | 164 | 0.18 | 1.36 | −1.21 | 2.42 | 8.236 | 2.3500 |
| 354153 | 0.22 | 1.68 | −0.20 | 1.62 | −0.83 | 0.94 | 164 | 0.75 | 0.53 | 0.20 | 0.58 | 4.838 | 2.3430 |
| 345586 | −0.86 | 0.83 | −0.23 | 1.86 | 0.24 | 1.52 | 163 | 0.26 | −0.55 | −0.15 | −0.59 | 5.273 | 2.3370 |
| 343487 | 0.11 | 1.65 | −0.85 | 1.11 | 0.00 | 0.00 | 164 | 0.93 | 0.06 | −16.45 | −0.73 | 4.772 | 2.3350 |
| 342907 | 0.29 | 1.66 | −0.50 | 1.35 | −1.10 | 1.29 | 160 | 0.80 | 0.70 | −0.14 | 0.64 | 5.361 | 2.3340 |
| 343403 | 0.94 | 1.87 | −0.31 | 1.27 | −0.33 | 1.63 | 131 | 0.41 | 0.64 | −0.97 | 0.74 | 4.704 | 2.3330 |
| 352421 | −0.87 | 1.06 | −0.14 | 1.48 | 0.16 | 1.65 | 160 | 0.32 | −0.52 | −0.42 | −0.59 | 5.029 | 2.3330 |
| 349751 | −0.74 | 1.06 | 0.25 | 1.79 | 0.04 | 1.60 | 158 | 0.39 | −0.39 | −1.54 | −0.53 | 4.781 | 2.3320 |
| 346635 | −1.59 | 0.12 | −1.02 | 1.03 | 0.07 | 1.62 | 161 | 0.05 | −0.83 | 0.31 | −0.59 | 7.635 | 2.3290 |
| 347231 | −0.85 | 1.40 | −0.02 | 1.70 | 0.24 | 1.57 | 156 | 0.33 | −0.54 | −0.52 | −0.64 | 4.682 | 2.3270 |
| 342547 | 0.00 | 0.00 | −0.80 | 1.24 | 0.16 | 1.67 | 134 | 0.10 | −0.08 | 11.00 | 0.63 | 4.513 | 2.3260 |
| 349258 | −0.83 | 0.94 | −0.24 | 1.66 | 0.27 | 1.67 | 157 | 0.25 | −0.55 | −0.07 | −0.57 | 4.889 | 2.3250 |
| 347326 | 0.05 | 1.60 | 0.00 | 0.00 | −1.84 | 0.91 | 164 | 0.96 | 0.95 | 0.95 | 1.77 | 6.795 | 2.3240 |
| 343688 | −1.22 | 1.26 | −1.08 | 1.10 | 0.29 | 1.62 | 160 | 0.14 | −0.76 | 0.81 | −0.31 | 5.489 | 2.3230 |
| 350637 | 0.05 | 1.65 | −0.95 | 0.55 | 0.00 | 0.00 | 164 | 0.96 | 0.03 | −39.00 | −0.88 | 6.000 | 2.3160 |
| 349966 | −0.44 | 1.44 | −0.02 | 1.54 | 0.59 | 1.82 | 164 | 0.55 | −0.52 | 0.18 | −0.52 | 4.550 | 2.3120 |
| 344729 | 0.09 | 1.67 | 0.23 | 1.73 | −0.71 | 1.02 | 164 | 0.55 | 0.40 | 1.35 | 0.45 | 4.607 | 2.3120 |
| 353250 | 0.06 | 1.50 | −0.01 | 1.92 | −2.07 | 0.90 | 163 | 0.86 | 1.06 | 0.93 | 1.77 | 6.980 | 2.3090 |
| 351265 | 0.37 | 1.86 | −0.25 | 1.39 | −0.69 | 1.11 | 155 | 0.69 | 0.53 | −0.17 | 0.50 | 4.940 | 2.3080 |
| 351316 | 0.13 | 1.60 | 0.06 | 1.75 | −0.95 | 1.04 | 163 | 0.71 | 0.54 | 0.87 | 0.74 | 5.008 | 2.3050 |
| 354068 | 0.17 | 1.57 | −0.10 | 1.85 | −0.95 | 0.77 | 160 | 0.76 | 0.56 | 0.52 | 0.71 | 5.205 | 2.3030 |
| 349875 | −0.43 | 1.44 | 0.42 | 1.75 | 0.90 | 1.50 | 164 | 0.73 | −0.67 | −0.28 | −0.58 | 5.086 | 2.2990 |
| 344805 | −1.42 | 1.02 | −0.88 | 1.11 | 0.18 | 1.64 | 162 | 0.10 | −0.80 | 0.33 | −0.59 | 5.649 | 2.2980 |
| 353521 | 0.23 | 1.79 | −0.09 | 1.29 | −0.89 | 1.25 | 160 | 0.74 | 0.56 | 0.43 | 0.68 | 4.979 | 2.2950 |
| 345188 | 0.35 | 1.87 | 0.00 | 0.00 | −0.37 | 1.20 | 154 | 0.53 | 0.36 | 0.03 | 0.36 | 4.046 | 2.2880 |
| 351548 | 0.00 | 0.00 | −1.07 | 1.04 | 0.07 | 1.63 | 164 | 0.04 | −0.04 | 31.57 | 0.98 | 5.083 | 2.2870 |
| 352912 | −1.20 | 0.93 | 0.13 | 1.98 | 0.08 | 1.55 | 150 | 0.21 | −0.64 | −1.08 | −1.04 | 5.141 | 2.2870 |
| 348573 | 0.50 | 1.71 | 0.00 | 0.00 | −0.27 | 1.51 | 163 | 0.34 | 0.39 | −0.30 | 0.42 | 4.036 | 2.2840 |
| 352208 | 0.04 | 1.53 | 0.31 | 2.01 | −1.44 | 0.85 | 144 | 0.85 | 0.74 | 1.36 | 1.44 | 5.964 | 2.2790 |
| 348753 | 0.19 | 1.74 | −0.81 | 1.13 | 0.00 | 0.00 | 141 | 0.92 | 0.10 | −9.53 | −0.66 | 4.646 | 2.2770 |
| 351603 | −0.47 | 1.25 | 0.14 | 1.73 | 0.63 | 1.89 | 163 | 0.61 | −0.55 | −0.11 | −0.54 | 4.542 | 2.2720 |
| 353209 | 0.10 | 1.63 | −1.39 | 0.89 | −1.26 | 0.45 | 164 | 0.95 | 0.68 | −1.19 | −0.05 | 6.507 | 2.2710 |
| 344344 | −0.52 | 1.45 | −0.16 | 1.24 | 0.49 | 1.85 | 147 | 0.44 | −0.51 | 0.29 | −0.49 | 4.497 | 2.2690 |
| 349919 | 0.12 | 1.68 | −0.72 | 1.12 | 0.00 | 0.00 | 162 | 0.92 | 0.06 | −13.00 | −0.59 | 4.486 | 2.2690 |
| 347248 | −0.45 | 1.07 | 0.48 | 1.76 | −0.19 | 1.72 | 161 | 0.47 | −0.13 | −6.15 | −0.18 | 4.462 | 2.2640 |
| 352103 | 0.11 | 1.67 | 0.00 | 0.00 | −0.72 | 0.95 | 162 | 0.87 | 0.41 | 0.73 | 0.64 | 4.635 | 2.2610 |
| 344847 | −1.05 | 0.88 | −0.37 | 1.53 | 0.19 | 1.66 | 162 | 0.19 | −0.62 | −0.10 | −0.66 | 5.465 | 2.2600 |
| 347291 | 0.21 | 1.53 | −0.38 | 1.99 | −1.06 | 0.78 | 163 | 0.82 | 0.64 | 0.07 | 0.66 | 5.104 | 2.2570 |
| 354317 | 0.50 | 1.72 | 0.04 | 1.63 | −0.53 | 1.42 | 158 | 0.44 | 0.52 | 0.11 | 0.51 | 4.508 | 2.2540 |
| 352932 | 0.15 | 1.62 | 0.07 | 1.60 | −1.08 | 1.38 | 164 | 0.71 | 0.61 | 0.87 | 0.84 | 5.054 | 2.2500 |
| 346988 | 0.00 | 0.00 | −0.74 | 1.27 | 0.13 | 1.65 | 164 | 0.09 | −0.07 | 12.38 | 0.60 | 4.372 | 2.2460 |
| 353421 | 0.21 | 1.67 | −0.04 | 1.65 | −0.91 | 1.06 | 160 | 0.70 | 0.56 | 0.55 | 0.68 | 5.102 | 2.2440 |
| 344698 | −0.11 | 1.51 | 0.57 | 1.93 | −1.05 | 1.08 | 163 | 0.83 | 0.47 | 2.45 | 1.24 | 5.589 | 2.2430 |
| 352458 | 0.00 | 0.00 | 0.40 | 1.86 | −0.34 | 1.31 | 158 | 0.22 | 0.17 | 3.35 | −0.15 | 3.948 | 2.2410 |
| 342957 | 0.17 | 1.66 | −0.28 | 1.55 | −1.19 | 0.90 | 163 | 0.81 | 0.68 | 0.34 | 0.82 | 5.738 | 2.2410 |
| 347227 | 0.41 | 1.84 | −0.28 | 1.51 | −0.48 | 1.02 | 164 | 0.61 | 0.44 | −0.55 | 0.39 | 4.514 | 2.2400 |
| 350468 | −0.90 | 0.52 | −0.19 | 1.62 | 0.16 | 1.66 | 164 | 0.22 | −0.53 | −0.34 | −0.63 | 5.638 | 2.2360 |
| 345974 | −0.93 | 0.84 | −0.61 | 1.20 | 0.30 | 1.73 | 164 | 0.19 | −0.61 | 0.48 | −0.43 | 5.756 | 2.2340 |
| 343714 | −1.06 | 0.92 | 0.00 | 0.00 | 0.06 | 1.65 | 162 | 0.07 | −0.56 | −0.89 | −0.99 | 5.133 | 2.2340 |
| 344679 | 0.00 | 0.00 | −1.68 | 1.01 | 0.06 | 1.61 | 163 | 0.02 | −0.03 | 57.00 | 1.61 | 6.085 | 2.2340 |
| 343947 | 0.29 | 1.79 | −0.01 | 1.46 | −0.88 | 1.50 | 147 | 0.69 | 0.58 | 0.49 | 0.70 | 4.697 | 2.2280 |
| 347761 | −0.14 | 1.70 | 0.55 | 1.78 | −0.41 | 0.75 | 163 | 0.69 | 0.13 | 6.11 | 0.45 | 4.592 | 2.2250 |
| 344702 | 0.14 | 1.67 | −0.29 | 1.58 | −0.86 | 0.69 | 160 | 0.78 | 0.50 | 0.14 | 0.54 | 4.998 | 2.2150 |
| 350638 | −0.75 | 1.07 | 0.24 | 1.66 | −0.04 | 1.67 | 164 | 0.36 | −0.35 | −1.79 | −0.54 | 4.787 | 2.2130 |
| 346308 | 0.14 | 1.69 | −0.62 | 1.21 | 0.00 | 0.00 | 164 | 0.88 | 0.07 | −9.86 | −0.46 | 4.155 | 2.2110 |
| 346575 | 0.15 | 1.60 | −0.19 | 1.63 | −1.66 | 1.26 | 161 | 0.85 | 0.90 | 0.62 | 1.31 | 5.694 | 2.2100 |
| 347113 | −0.04 | 1.67 | 0.24 | 1.66 | −0.75 | 1.07 | 164 | 0.64 | 0.35 | 1.79 | 0.54 | 4.787 | 2.2090 |
| 354375 | −0.83 | 0.94 | −0.16 | 1.63 | 0.21 | 1.69 | 162 | 0.25 | −0.52 | −0.29 | −0.60 | 4.735 | 2.2080 |
| 351503 | −0.89 | 0.83 | −0.24 | 1.82 | 0.24 | 1.52 | 164 | 0.26 | −0.56 | −0.15 | −0.61 | 5.317 | 2.2040 |
| 344242 | 0.28 | 1.67 | −0.14 | 1.57 | −0.77 | 1.32 | 161 | 0.68 | 0.53 | 0.20 | 0.56 | 4.716 | 2.2020 |
| 350057 | 0.54 | 1.89 | −0.04 | 1.63 | −0.46 | 1.14 | 159 | 0.51 | 0.50 | −0.16 | 0.50 | 4.480 | 2.2000 |
| 346870 | −0.22 | 1.54 | −0.40 | 1.35 | 0.55 | 1.81 | 163 | 0.46 | −0.39 | 1.47 | −0.34 | 4.422 | 2.1970 |
| 346467 | 0.19 | 1.72 | −0.36 | 1.24 | −0.92 | 1.07 | 164 | 0.81 | 0.56 | 0.01 | 0.56 | 4.964 | 2.1970 |
| 352828 | 0.39 | 1.84 | −0.12 | 1.44 | −0.57 | 1.36 | 163 | 0.59 | 0.48 | −0.06 | 0.47 | 4.485 | 2.1970 |
| 348766 | 0.05 | 1.72 | 0.60 | 1.21 | −0.76 | 1.00 | 158 | 0.86 | 0.41 | 2.36 | 1.09 | 5.111 | 2.1970 |
| 344568 | 0.07 | 1.63 | −1.19 | 1.08 | 0.00 | 0.00 | 164 | 0.96 | 0.04 | −35.00 | −1.09 | 5.125 | 2.1960 |
| 345024 | 0.35 | 1.73 | −0.11 | 1.66 | −0.59 | 1.09 | 159 | 0.62 | 0.47 | 0.02 | 0.47 | 4.576 | 2.1950 |
| 347818 | 0.19 | 1.65 | −1.17 | 1.34 | −0.83 | 0.70 | 155 | 0.89 | 0.51 | −1.67 | −0.16 | 5.475 | 2.1940 |
| 343584 | 0.16 | 1.62 | −0.17 | 1.55 | −1.42 | 1.27 | 164 | 0.82 | 0.79 | 0.58 | 1.08 | 5.488 | 2.1920 |
| 344655 | 1.04 | 1.72 | −0.26 | 1.42 | −0.09 | 1.66 | 163 | 0.31 | 0.57 | −1.30 | 0.84 | 4.856 | 2.1890 |
| 346809 | 0.18 | 1.60 | −1.56 | 1.28 | −1.13 | 0.54 | 158 | 0.94 | 0.65 | −1.66 | −0.29 | 6.322 | 2.1890 |
| 354353 | 0.51 | 1.73 | −0.07 | 1.71 | −0.45 | 1.32 | 159 | 0.50 | 0.48 | −0.21 | 0.48 | 4.345 | 2.1880 |
| 348228 | 0.10 | 1.52 | −0.07 | 1.88 | −1.84 | 0.99 | 163 | 0.85 | 0.97 | 0.82 | 1.53 | 6.354 | 2.1840 |
| 352966 | −0.18 | 1.60 | 0.80 | 1.54 | 0.00 | 0.00 | 164 | 0.91 | −0.09 | −9.89 | 0.63 | 4.231 | 2.1820 |
| 348933 | 0.00 | 1.77 | 0.39 | 1.14 | −0.54 | 0.77 | 164 | 0.81 | 0.27 | 2.44 | 0.68 | 4.760 | 2.1820 |
| 346587 | 0.20 | 1.57 | −0.29 | 1.85 | −0.98 | 0.95 | 162 | 0.79 | 0.59 | 0.17 | 0.65 | 4.906 | 2.1800 |
| 352387 | 0.30 | 1.55 | 0.02 | 1.75 | −0.88 | 1.17 | 135 | 0.68 | 0.59 | 0.53 | 0.70 | 4.591 | 2.1790 |
| 354213 | 0.22 | 1.85 | −0.35 | 1.16 | −0.70 | 0.69 | 162 | 0.77 | 0.46 | −0.24 | 0.40 | 5.137 | 2.1770 |
| 349413 | 0.14 | 1.58 | 0.05 | 1.75 | −0.92 | 1.11 | 164 | 0.71 | 0.53 | 0.83 | 0.72 | 4.848 | 2.1770 |
| 350451 | −0.31 | 1.39 | 0.00 | 0.00 | 0.41 | 1.80 | 163 | 0.57 | −0.36 | 0.14 | −0.37 | 3.932 | 2.1760 |
| 350348 | −0.29 | 1.56 | −0.35 | 1.26 | 0.65 | 1.92 | 151 | 0.54 | −0.47 | 1.13 | −0.52 | 4.360 | 2.1750 |
| 348940 | 0.07 | 1.63 | −1.19 | 1.08 | 0.00 | 0.00 | 163 | 0.96 | 0.04 | −35.00 | −1.09 | 5.094 | 2.1740 |
| 343982 | 0.12 | 1.71 | −0.02 | 1.67 | −1.03 | 0.74 | 153 | 0.74 | 0.57 | 0.76 | 0.78 | 5.466 | 2.1720 |
| 346260 | 0.07 | 1.63 | −1.19 | 1.08 | 0.00 | 0.00 | 164 | 0.96 | 0.04 | −35.00 | −1.09 | 5.125 | 2.1710 |
| 344950 | 0.14 | 1.62 | 0.00 | 0.00 | −0.81 | 1.45 | 162 | 0.83 | 0.48 | 0.71 | 0.70 | 4.269 | 2.1700 |
| 353131 | 0.41 | 1.70 | −0.14 | 1.67 | −0.52 | 1.30 | 162 | 0.57 | 0.46 | −0.18 | 0.45 | 4.405 | 2.1690 |
| 350773 | −0.62 | 1.46 | −0.23 | 1.48 | 0.39 | 1.71 | 163 | 0.36 | −0.51 | 0.23 | −0.47 | 4.520 | 2.1640 |
| 345050 | 0.22 | 1.76 | 0.10 | 1.54 | −0.71 | 1.28 | 163 | 0.61 | 0.46 | 0.74 | 0.54 | 4.412 | 2.1640 |
| 342822 | −0.44 | 1.37 | 0.60 | 1.95 | −0.17 | 1.20 | 155 | 0.65 | −0.14 | −6.70 | 0.15 | 4.635 | 2.1620 |
| 347228 | 0.43 | 1.86 | −0.36 | 1.49 | −0.46 | 1.02 | 161 | 0.61 | 0.45 | −0.78 | 0.37 | 4.427 | 2.1620 |
| 351815 | −0.73 | 0.65 | 0.00 | 0.00 | 0.05 | 1.69 | 159 | 0.08 | −0.39 | −0.87 | −0.67 | 4.868 | 2.1590 |
| 350759 | 0.46 | 1.70 | −0.37 | 1.47 | −0.50 | 1.48 | 161 | 0.62 | 0.48 | −0.73 | 0.40 | 4.417 | 2.1580 |
| 352407 | −0.63 | 1.46 | 0.28 | 1.88 | 0.11 | 1.25 | 164 | 0.47 | −0.37 | −1.46 | −0.41 | 4.359 | 2.1560 |
| 352334 | −0.61 | 1.17 | 0.01 | 1.66 | 0.35 | 1.74 | 157 | 0.42 | −0.48 | −0.29 | −0.50 | 4.485 | 2.1520 |
| 342901 | 0.10 | 1.69 | −0.02 | 1.67 | −1.03 | 0.74 | 157 | 0.75 | 0.57 | 0.79 | 0.78 | 5.395 | 2.1460 |
| 354028 | −0.64 | 1.15 | 0.31 | 1.62 | −0.05 | 1.71 | 164 | 0.36 | −0.29 | −2.22 | −0.48 | 4.543 | 2.1460 |
| 347158 | 0.47 | 1.56 | 0.05 | 1.42 | −0.52 | 1.79 | 156 | 0.45 | 0.49 | 0.15 | 0.49 | 4.376 | 2.1430 |
| 353491 | 0.27 | 1.74 | −0.37 | 1.50 | −0.84 | 0.66 | 143 | 0.78 | 0.55 | −0.15 | 0.51 | 5.331 | 2.1400 |
| 344932 | −0.55 | 1.37 | 0.37 | 1.85 | 0.11 | 1.46 | 162 | 0.52 | −0.33 | −1.79 | −0.30 | 4.323 | 2.1400 |
| 353863 | 0.17 | 1.43 | −0.06 | 2.11 | −1.34 | 1.33 | 162 | 0.81 | 0.76 | 0.70 | 1.08 | 4.930 | 2.1340 |
| 346945 | 0.05 | 1.66 | −0.83 | 0.62 | 0.00 | 0.00 | 162 | 0.96 | 0.03 | −34.20 | −0.77 | 5.215 | 2.1330 |
| 353009 | −0.57 | 1.44 | −0.20 | 1.54 | 0.38 | 1.70 | 161 | 0.39 | −0.47 | 0.22 | −0.45 | 4.375 | 2.1320 |
| 345473 | −1.63 | 0.40 | 0.06 | 1.81 | 0.01 | 1.59 | 161 | 0.11 | −0.82 | −1.06 | −1.50 | 6.959 | 2.1300 |
| 346419 | −0.87 | 0.77 | −0.04 | 1.62 | 0.14 | 1.69 | 164 | 0.28 | −0.51 | −0.64 | −0.65 | 5.118 | 2.1290 |
| 350446 | 0.06 | 1.62 | −0.67 | 1.05 | 0.00 | 0.00 | 164 | 0.91 | 0.03 | −23.33 | −0.54 | 4.191 | 2.1260 |
| 352587 | 0.24 | 1.66 | −0.84 | 1.01 | −2.05 | 0.70 | 164 | 0.88 | 1.15 | 0.06 | 1.19 | 8.493 | 2.1240 |
| 348912 | −0.01 | 1.73 | 0.26 | 1.17 | −0.85 | 0.78 | 162 | 0.86 | 0.42 | 1.64 | 0.92 | 5.141 | 2.1220 |
| 348625 | −1.12 | 0.50 | −0.82 | 1.06 | 0.13 | 1.67 | 163 | 0.09 | −0.62 | 0.52 | −0.36 | 6.061 | 2.1200 |
| 347384 | −0.43 | 1.49 | −0.02 | 1.57 | 0.78 | 1.86 | 146 | 0.62 | −0.60 | 0.32 | −0.65 | 4.513 | 2.1190 |
| 350542 | −1.86 | 0.88 | −0.66 | 1.12 | 0.18 | 1.66 | 164 | 0.11 | −1.02 | −0.18 | −1.16 | 6.938 | 2.1160 |
| 349070 | −0.20 | 1.72 | 0.09 | 1.37 | 1.44 | 1.18 | 162 | 0.78 | −0.82 | 0.65 | −1.12 | 5.815 | 2.1130 |
| 353215 | 0.43 | 1.55 | −0.08 | 1.66 | −0.57 | 1.48 | 164 | 0.56 | 0.50 | −0.02 | 0.50 | 4.451 | 2.1110 |
| 353840 | 0.80 | 1.41 | 0.28 | 1.80 | −0.43 | 1.39 | 164 | 0.30 | 0.61 | 0.15 | 0.58 | 4.911 | 2.1060 |
| 353031 | −0.92 | 0.89 | −0.37 | 1.48 | 0.28 | 1.62 | 150 | 0.27 | −0.60 | 0.08 | −0.58 | 5.412 | 2.1060 |
| 346304 | −0.68 | 1.02 | −0.32 | 1.52 | 0.40 | 1.85 | 146 | 0.32 | −0.54 | 0.33 | −0.47 | 4.828 | 2.1060 |
| 350276 | 0.05 | 1.61 | −1.77 | 0.98 | 0.00 | 0.00 | 161 | 0.98 | 0.03 | −71.80 | −1.70 | 6.131 | 2.1040 |
| 346298 | −1.16 | 0.50 | −0.04 | 1.60 | 0.03 | 1.61 | 158 | 0.17 | −0.59 | −0.88 | −0.94 | 5.884 | 2.1020 |
| 351893 | −0.19 | 1.59 | −0.02 | 1.61 | 1.20 | 1.48 | 163 | 0.75 | −0.69 | 0.76 | −0.95 | 5.032 | 2.1010 |
| 344373 | 0.28 | 1.85 | −0.20 | 1.26 | −0.67 | 1.17 | 158 | 0.71 | 0.48 | −0.01 | 0.47 | 4.564 | 2.0990 |
| 352053 | 0.00 | 0.00 | −0.64 | 1.28 | 0.15 | 1.66 | 164 | 0.10 | −0.07 | 9.53 | 0.49 | 4.089 | 2.0950 |
| 345762 | −1.59 | 0.12 | −0.80 | 1.38 | 0.07 | 1.62 | 163 | 0.06 | −0.83 | 0.05 | −0.79 | 7.244 | 2.0940 |
| 349103 | −0.87 | 0.77 | −0.07 | 1.63 | 0.14 | 1.69 | 163 | 0.28 | −0.51 | −0.58 | −0.64 | 5.099 | 2.0930 |
| 352323 | −0.46 | 1.44 | 0.10 | 1.64 | 0.62 | 1.72 | 161 | 0.61 | −0.54 | −0.04 | −0.54 | 4.468 | 2.0920 |
| 351310 | 0.02 | 1.63 | −1.13 | 0.32 | 0.00 | 0.00 | 159 | 0.98 | 0.01 | −114.00 | −1.09 | 7.870 | 2.0920 |
| 344465 | 1.38 | 1.69 | 0.17 | 1.78 | −0.23 | 1.49 | 129 | 0.33 | 0.80 | −0.50 | 0.94 | 4.984 | 2.0920 |
| 349048 | 0.06 | 1.63 | −0.86 | 0.86 | 0.00 | 0.00 | 157 | 0.95 | 0.03 | −29.67 | −0.77 | 4.737 | 2.0910 |
| 350254 | 0.39 | 1.83 | −0.16 | 1.58 | −0.51 | 1.06 | 163 | 0.60 | 0.45 | −0.22 | 0.43 | 4.390 | 2.0900 |
| 353084 | −2.05 | 0.70 | −0.83 | 1.05 | 0.21 | 1.65 | 164 | 0.11 | −1.13 | −0.08 | −1.20 | 8.289 | 2.0900 |
| 353496 | 0.36 | 1.77 | −0.16 | 1.34 | −0.86 | 1.45 | 140 | 0.74 | 0.61 | 0.15 | 0.65 | 4.658 | 2.0890 |
| 352532 | −0.40 | 1.34 | −0.02 | 1.62 | 0.57 | 1.86 | 164 | 0.57 | −0.48 | 0.22 | −0.50 | 4.294 | 2.0870 |
| 351762 | 0.14 | 1.60 | −0.86 | 1.47 | 0.00 | 0.00 | 164 | 0.92 | 0.07 | −13.29 | −0.72 | 4.248 | 2.0860 |
| 345252 | −0.53 | 1.16 | 0.24 | 1.65 | 0.46 | 1.97 | 164 | 0.57 | −0.49 | −0.56 | −0.46 | 4.305 | 2.0850 |
| 353416 | −0.94 | 0.71 | 0.27 | 1.52 | 0.06 | 1.84 | 131 | 0.21 | −0.50 | −1.42 | −0.91 | 5.316 | 2.0850 |
| 346615 | −0.84 | 0.99 | −0.57 | 1.59 | 0.32 | 1.61 | 163 | 0.21 | −0.58 | 0.53 | −0.40 | 5.037 | 2.0810 |
| 348528 | 0.00 | 0.00 | −0.84 | 1.34 | 0.12 | 1.64 | 161 | 0.08 | −0.06 | 15.00 | 0.69 | 4.261 | 2.0800 |
| 348154 | −0.57 | 1.19 | −0.08 | 1.67 | 0.33 | 1.70 | 164 | 0.41 | −0.45 | −0.09 | −0.46 | 4.372 | 2.0790 |
| 347665 | 0.27 | 1.66 | −0.40 | 1.47 | −1.26 | 1.08 | 162 | 0.80 | 0.77 | 0.12 | 0.82 | 5.703 | 2.0790 |
| 351212 | 0.11 | 1.63 | −0.92 | 1.12 | 0.00 | 0.00 | 160 | 0.94 | 0.06 | −17.73 | −0.80 | 4.539 | 2.0770 |
| 348215 | 0.31 | 1.86 | 0.03 | 1.59 | −0.57 | 1.03 | 164 | 0.58 | 0.44 | 0.36 | 0.46 | 4.289 | 2.0770 |
| 354281 | 0.40 | 1.65 | 0.00 | 1.64 | −0.57 | 1.50 | 157 | 0.56 | 0.48 | 0.18 | 0.50 | 4.304 | 2.0750 |
| 348201 | −0.07 | 1.66 | 0.55 | 1.59 | −0.68 | 0.79 | 161 | 0.86 | 0.31 | 3.03 | 0.97 | 4.901 | 2.0720 |
| 342867 | 0.14 | 1.54 | 0.05 | 1.78 | −1.17 | 1.21 | 157 | 0.74 | 0.66 | 0.86 | 0.92 | 5.115 | 2.0720 |
| 351675 | −0.61 | 1.60 | 0.40 | 1.86 | 0.07 | 1.43 | 155 | 0.40 | −0.34 | −1.97 | −0.47 | 4.288 | 2.0710 |
| 349730 | 0.13 | 1.55 | −0.45 | 1.98 | −1.65 | 0.96 | 164 | 0.90 | 0.89 | 0.35 | 1.14 | 5.127 | 2.0570 |
| 350739 | −0.68 | 0.79 | 0.55 | 1.59 | −0.08 | 1.65 | 163 | 0.14 | −0.30 | −3.10 | −0.97 | 4.909 | 2.0570 |
| 347606 | −0.99 | 1.73 | −0.01 | 1.52 | 0.19 | 1.57 | 162 | 0.30 | −0.59 | −0.66 | −0.75 | 4.565 | 2.0570 |
| 349777 | 0.43 | 1.87 | −0.33 | 1.46 | −0.47 | 1.13 | 163 | 0.58 | 0.45 | −0.69 | 0.40 | 4.326 | 2.0560 |
| 346705 | 0.08 | 1.68 | −0.71 | 0.99 | 0.00 | 0.00 | 162 | 0.93 | 0.04 | −18.75 | −0.61 | 4.270 | 2.0550 |
| 353927 | 0.23 | 1.63 | 0.03 | 1.62 | −0.85 | 1.41 | 163 | 0.64 | 0.54 | 0.63 | 0.63 | 4.591 | 2.0540 |
| 345229 | 0.55 | 1.88 | 0.24 | 1.48 | −0.60 | 1.52 | 162 | 0.39 | 0.57 | 0.46 | 0.51 | 4.429 | 2.0540 |
| 343365 | 0.11 | 1.47 | 0.13 | 2.03 | −1.04 | 1.12 | 157 | 0.76 | 0.58 | 1.03 | 0.88 | 4.575 | 2.0540 |
| 350091 | −0.50 | 1.21 | 0.07 | 1.72 | 0.44 | 1.82 | 161 | 0.53 | −0.47 | −0.21 | −0.46 | 4.227 | 2.0530 |
| 348155 | −0.57 | 1.19 | −0.08 | 1.68 | 0.33 | 1.70 | 163 | 0.40 | −0.45 | −0.09 | −0.46 | 4.347 | 2.0520 |
| 352560 | −1.38 | 0.37 | −0.61 | 1.02 | 0.11 | 1.69 | 159 | 0.08 | −0.74 | −0.03 | −0.77 | 7.345 | 2.0510 |
| 353575 | 0.25 | 1.65 | −0.48 | 1.38 | −0.99 | 1.35 | 164 | 0.80 | 0.62 | −0.18 | 0.55 | 4.774 | 2.0460 |
| 352524 | −1.51 | 0.37 | −0.25 | 1.85 | 0.08 | 1.57 | 164 | 0.12 | −0.80 | −0.58 | −1.15 | 6.862 | 2.0450 |
| 349152 | −0.81 | 0.93 | −0.04 | 1.94 | 0.27 | 1.48 | 143 | 0.28 | −0.54 | −0.43 | −0.64 | 4.693 | 2.0430 |
| 350072 | 0.26 | 1.66 | 0.07 | 1.63 | −0.67 | 1.40 | 163 | 0.57 | 0.47 | 0.59 | 0.51 | 4.299 | 2.0390 |
| 347218 | 0.49 | 1.84 | −0.45 | 1.29 | −0.18 | 1.49 | 161 | 0.58 | 0.33 | −1.81 | 0.23 | 4.301 | 2.0390 |
| 353817 | 0.21 | 1.55 | −0.07 | 1.83 | −0.78 | 1.08 | 164 | 0.70 | 0.49 | 0.43 | 0.58 | 4.595 | 2.0340 |
| 352337 | −0.99 | 1.59 | −0.17 | 1.67 | 0.26 | 1.54 | 163 | 0.26 | −0.62 | −0.31 | −0.72 | 4.662 | 2.0320 |
| 344120 | 0.19 | 1.58 | 0.02 | 1.77 | −0.89 | 1.05 | 144 | 0.66 | 0.54 | 0.69 | 0.66 | 4.752 | 2.0320 |
| 349153 | 0.53 | 1.77 | −0.40 | 1.37 | −0.39 | 1.48 | 164 | 0.60 | 0.46 | −1.02 | 0.37 | 4.325 | 2.0280 |
| 349345 | 0.12 | 1.63 | −0.70 | 1.40 | 0.00 | 0.00 | 163 | 0.91 | 0.06 | −12.67 | −0.56 | 4.004 | 2.0270 |
| 353791 | 0.29 | 1.70 | −0.60 | 1.13 | −1.18 | 1.46 | 148 | 0.85 | 0.73 | −0.21 | 0.63 | 4.878 | 2.0260 |
| 350042 | 0.00 | 0.00 | −1.53 | 0.53 | 0.03 | 1.63 | 163 | 0.01 | −0.01 | 103.00 | 1.49 | 7.500 | 2.0260 |
| 347461 | −2.12 | 0.52 | 0.14 | 2.02 | 0.00 | 1.47 | 159 | 0.15 | −1.06 | −1.13 | −1.91 | 8.552 | 2.0250 |
| 351745 | 0.00 | 0.00 | −0.72 | 0.20 | 0.20 | 1.63 | 129 | 0.03 | −0.10 | 8.20 | 0.67 | 7.274 | 2.0220 |
| 350217 | 0.15 | 1.61 | −0.80 | 1.49 | 0.00 | 0.00 | 146 | 0.90 | 0.07 | −11.67 | −0.63 | 4.055 | 2.0210 |
| 347599 | −0.06 | 1.33 | 0.31 | 1.75 | −1.00 | 1.78 | 163 | 0.66 | 0.47 | 1.79 | 0.74 | 4.683 | 2.0210 |
| 345617 | −0.76 | 0.52 | 0.04 | 0.87 | 0.04 | 1.75 | 164 | 0.13 | −0.40 | −1.00 | −0.70 | 4.746 | 2.0200 |
| 344362 | 0.00 | 0.00 | −0.92 | 1.12 | 0.10 | 1.63 | 164 | 0.06 | −0.05 | 19.40 | 0.81 | 4.506 | 2.0190 |
| 352640 | 0.20 | 1.53 | −0.36 | 2.01 | −1.08 | 0.83 | 160 | 0.83 | 0.64 | 0.13 | 0.69 | 4.864 | 2.0170 |
| 346612 | 0.64 | 1.89 | −0.33 | 1.51 | −0.13 | 1.43 | 163 | 0.45 | 0.39 | −1.52 | 0.44 | 4.246 | 2.0170 |
| 343513 | −0.10 | 0.85 | −0.84 | 0.88 | 0.05 | 1.83 | 140 | 0.20 | −0.08 | 10.87 | 0.42 | 4.585 | 2.0160 |
| 347501 | 0.02 | 1.78 | −0.35 | 1.17 | 1.01 | 1.92 | 164 | 0.68 | −0.49 | 1.75 | −0.81 | 4.656 | 2.0160 |
| 350426 | −0.59 | 0.91 | 0.38 | 1.63 | 0.04 | 2.09 | 143 | 0.47 | −0.32 | −2.08 | −0.35 | 4.218 | 2.0150 |
| 345142 | 0.27 | 1.76 | 0.12 | 1.68 | −0.63 | 1.20 | 161 | 0.55 | 0.45 | 0.67 | 0.48 | 4.231 | 2.0130 |
| 353319 | −0.94 | 1.15 | 0.16 | 1.69 | 0.01 | 1.60 | 164 | 0.31 | −0.47 | −1.32 | −0.71 | 4.801 | 2.0130 |
| 345639 | −0.83 | 0.64 | −0.07 | 1.44 | 0.06 | 1.72 | 162 | 0.22 | −0.44 | −0.71 | −0.62 | 4.778 | 2.0120 |
| 352578 | 0.30 | 1.64 | −0.24 | 1.69 | −0.81 | 1.24 | 156 | 0.72 | 0.56 | 0.03 | 0.56 | 4.617 | 2.0100 |
| 343981 | 0.13 | 1.63 | 0.07 | 1.62 | −1.00 | 0.77 | 158 | 0.77 | 0.56 | 0.89 | 0.83 | 5.282 | 2.0080 |
| 344472 | −0.25 | 1.34 | −0.30 | 1.27 | 0.65 | 2.02 | 156 | 0.49 | −0.45 | 1.11 | −0.44 | 4.187 | 2.0070 |
| 353187 | 0.00 | 0.00 | −1.04 | 1.39 | 0.11 | 1.60 | 164 | 0.05 | −0.06 | 19.91 | 0.92 | 4.397 | 2.0040 |
| 351780 | 0.01 | 1.60 | 0.16 | 1.69 | −0.94 | 1.15 | 164 | 0.69 | 0.47 | 1.32 | 0.71 | 4.801 | 2.0010 |
| 345444 | 0.03 | 1.65 | −0.94 | 0.56 | 0.00 | 0.00 | 162 | 0.98 | 0.01 | −63.67 | −0.90 | 5.480 | 2.0010 |

Locus is the ParAllele identifiers of the polymorphisms,
N is the number of genotypes,
mean_0 is the mean intramuscular fat for genotype 0,
mean_1 is the mean for genotype 1, and
mean_2 is the mean of genotype 2,
SD is the standard deviation,
a is the additive effect,
k is the dominance effect,
alpha is the average effect of allele substitutions,
tmax is the value of the t test, and
log(1/P) is the P value determined from 100,000 permutation tests expressed as a positive integer.
A minus value for a means the mean of genotype 0 is lower than the mean of genotype 2, and a minus value for alpha means that selecting for allele 0 will reduce the average values of intramuscular fat in the population.

TABLE 10
SNPs with a high probability of being associated with IMF. The idents
are those in Table 9 and are the ParAllele identifiers. These are located on the
BGSP scaffolds and contigs which indicate the SNP location. These scaffolds and
contigs have been graphically located to maps on the biolives website.

| Ident | scaffold | contig chr. | Gene comment | | SEQ ID NO |
|---|---|---|---|---|---|
| 343054 | SCAFFOLD317931_3159 | contig346200 | Bta13 or BTA-none | near TPT1 | 214 |
| 343362 | SCAFFOLD176082_5591 | contig172672 | Bta2 | GTF3C2 | 215 |
| 345497 | SCAFFOLD15887_443 | contig138107 | Bta2 | between LOC440930-ZNFN1A2 | 216 |
| 347422 | SCAFFOLD216467_6791 | contig11773 | Hsa12 | near SLC6A15 | 217 |
| 348950 | SCAFFOLD115159_16552 | contig68050 | Hsa10 | intron of CTNNA3 | 218 |
| 349339 | SCAFFOLD245307_20842 | contig21810 | Hsa5 | between EPB41LA and APC | 219 |
| 352076 | SCAFFOLD155567_7634 | contig352746 | Hsa5 | intron of DDX46 | 220 |
| 352376 | SCAFFOLD323635_4902 | contig252674 | Hsa1 | HS2ST1 not near RORC | 221 |
| 353331 | SCAFFOLD331833_481 | contig298758 | Bta-none | not recognised | 222 |
| 353833 | SCAFFOLD230335_25665 | contig19309 | Bta 5 or Bta-none | between LOC401178-CDH6 | 223 |

The sequence listing attached hereto gives DNA sequence for the contigs described in Table 10, and includes identification of the scaffold including the base pairs spanned by the contig and also identification of the position and nature of the polymorphism. For example, SEQ ID NO: 214 is for contig 346200, which spans scaffold 317931 from position 2394 to position 4575. In this instance the SNP is at position 3159, and the polymorphic form which favours intramuscular fat deposition is the form where base 3159 is A.

TABLE 11
Parallele identifiers for additional SNP associated with IMF (nir fat) showing
the sequence scaffold, the Baylor College of Medicine sequence contig containing the SNP,
the Genbank Accession of the DNA sequence, and the alternative bases for each SNP. The
exact location of each SNP is shown in the sequence scaffold, the number after the
underscore is the exact base pair from the start of the scaffold.

| | | Sequence | | Genbank | | SEQ ID |
|---|---|---|---|---|---|---|
| Ident | Ibiss4 | scaffold | Baylor Contig | Accession | SNP bases | NO: |
| 349269 | Null | SCAFFOLD224445_646 | Contig688345 | AAFC01688313 | A/G | 224 |
| 349388 | Null | SCAFFOLD261939_1026 | Contig290263 | AAFC01290254 | G/T | 225 |
| 351385 | Null | SCAFFOLD116234_3345 | Contig387450 | AAFC01387438 | C/T | 226 |
| 353697 | Null | SCAFFOLD75727_10176 | Contig497215 | AAFC01497199 | A/G | 227 |
| 342572 | Null | SCAFFOLD266585_13721 | Contig144982 | AAFC01144976 | C/G | 228 |
| 342814 | Null | SCAFFOLD10496_11993 | Contig353686 | AAFC01353676 | A/G | 229 |
| 342909 | Null | SCAFFOLD181448_3708 | Contig403002 | AAFC01402989 | G/T | 230 |
| 343414 | Null | SCAFFOLD205015_1947 | Contig519798 | AAFC01519777 | C/T | 231 |
| 343536 | Null | SCAFFOLD283786_511 | Contig642091 | AAFC01642063 | A/G | 232 |
| 343542 | Null | SCAFFOLD290008_39459 | Contig19542 | AAFC01019542 | C/T | 233 |
| 343610 | Null | SCAFFOLD322423_1597 | Contig155852 | AAFC01155846 | A/G | 234 |
| 343827 | Null | SCAFFOLD100229_4858 | Contig30336 | AAFC01030336 | C/T | 235 |
| 343943 | Null | SCAFFOLD155268_21318 | Contig69791 | AAFC01069790 | C/T | 236 |
| 344028 | Null | SCAFFOLD194940_1187 | Contig473431 | AAFC01473415 | C/T | 237 |
| 344286 | Null | SCAFFOLD315271_6985 | Contig129954 | AAFC01129949 | G/T | 238 |
| 344319 | Null | SCAFFOLD359101_685 | Contig643326 | AAFC01643298 | C/T | 239 |
| 344554 | Null | SCAFFOLD105489_6953 | Contig82349 | AAFC01082348 | C/T | 240 |
| 344636 | Null | SCAFFOLD140875_7990 | Contig143679 | AAFC01143674 | A/G | 241 |
| 344851 | Null | SCAFFOLD230819_6338 | Contig399586 | AAFC01399573 | A/G | 242 |
| 344969 | Null | SCAFFOLD270010_49832 | Contig260643 | AAFC01260634 | C/T | 243 |
| 345154 | Null | SCAFFOLD57440_852 | Contig623028 | AAFC01623000 | C/T | 244 |
| 345203 | Null | SCAFFOLD76111_6497 | Contig167797 | AAFC01167791 | G/T | 245 |
| 345544 | Null | SCAFFOLD171748_1363 | Contig32037 | AAFC01032036 | A/G | 246 |
| 345719 | Null | SCAFFOLD235985_4468 | Contig15384 | AAFC01015384 | C/T | 247 |
| 345797 | Null | SCAFFOLD260513_2368 | Contig235872 | AAFC01235865 | A/G | 248 |
| 346008 | Null | SCAFFOLD41387_6913 | Contig28445 | AAFC01028445 | A/G | 249 |
| 346040 | Null | SCAFFOLD5257_13662 | Contig239211 | AAFC01239204 | A/G | 250 |
| 346154 | Null | SCAFFOLD95032_25614 | Contig339474 | AAFC01339464 | A/G | 251 |
| 346417 | Null | SCAFFOLD170179_19975 | Contig32904 | AAFC01032903 | A/G | 252 |
| 346418 | Null | SCAFFOLD170189_10300 | Contig399829 | AAFC01399816 | C/T | 253 |
| 346520 | Null | SCAFFOLD205603_7508 | Contig73971 | AAFC01073970 | A/G | 254 |
| 346584 | Null | SCAFFOLD231067_8487 | Contig661341 | AAFC01661313 | C/T | 255 |
| 346701 | Null | SCAFFOLD275001_30112 | Contig164110 | AAFC01164104 | C/T | 256 |
| 346888 | Null | SCAFFOLD41654_3571 | Contig180954 | AAFC01180948 | A/G | 257 |
| 346905 | Null | SCAFFOLD50003_7361 | Contig263385 | AAFC01263376 | A/G | 258 |
| 347009 | Null | SCAFFOLD85346_9768 | Contig147150 | AAFC01147144 | A/G | 259 |
| 347100 | Null | SCAFFOLD102006_3674 | Contig179930 | AAFC01179924 | C/T | 260 |
| 347206 | Null | SCAFFOLD144700_3239 | Contig20365 | AAFC01020365 | C/T | 261 |
| 347642 | Null | SCAFFOLD286711_9315 | Contig36453 | AAFC01036452 | A/C | 262 |
| 347674 | Null | SCAFFOLD296243_14017 | Contig56939 | AAFC01056938 | C/T | 263 |
| 347705 | Null | SCAFFOLD306025_6030 | Contig121863 | AAFC01121858 | A/T | 264 |
| 347715 | Null | SCAFFOLD310358_2998 | Contig327981 | AAFC01327971 | G/T | 265 |
| 348202 | Null | SCAFFOLD153044_4148 | Contig163124 | AAFC01163118 | G/T | 266 |
| 348212 | Null | SCAFFOLD155358_14044 | Contig238647 | AAFC01238640 | C/T | 267 |
| 348323 | Null | SCAFFOLD200129_19087 | Contig91367 | AAFC01091365 | C/T | 268 |
| 348335 | Null | SCAFFOLD201086_2241 | Contig111786 | AAFC01111783 | A/T | 269 |
| 348372 | Null | SCAFFOLD215074_4772 | Contig362870 | AAFC01362860 | C/G | 270 |
| 348491 | Null | SCAFFOLD256751_6825 | Contig293941 | AAFC01293931 | A/G | 271 |
| 348656 | Null | SCAFFOLD316702_22782 | Contig360244 | AAFC01360234 | C/T | 272 |
| 348725 | Null | SCAFFOLD46484_3236 | Contig237587 | AAFC01237580 | C/T | 273 |
| 348758 | Null | SCAFFOLD57440_686 | Contig623028 | AAFC01623000 | C/G | 274 |
| 349071 | Null | SCAFFOLD155739_13472 | Contig48739 | AAFC01048738 | A/C | 275 |
| 349469 | Null | SCAFFOLD290227_13905 | Contig149729 | AAFC01149723 | C/T | 276 |
| 349644 | Null | SCAFFOLD57440_997 | Contig623028 | AAFC01623000 | C/T | 277 |
| 349668 | Null | SCAFFOLD66599_4817 | Contig332084 | AAFC01332074 | C/T | 278 |
| 350008 | Null | SCAFFOLD185315_13446 | Contig212367 | AAFC01212361 | A/G | 279 |
| 350037 | Null | SCAFFOLD195108_11452 | Contig467929 | AAFC01467914 | A/G | 280 |
| 350281 | Null | SCAFFOLD286032_4505 | Contig116512 | AAFC01116508 | A/C | 281 |
| 350377 | Null | SCAFFOLD321801_17599 | Contig357493 | AAFC01357483 | A/G | 282 |
| 350400 | Null | SCAFFOLD35711_6623 | Contig354565 | AAFC01354555 | A/G | 283 |
| 350424 | Null | SCAFFOLD46484_3646 | Contig237587 | AAFC01237580 | A/C | 284 |
| 350456 | Null | SCAFFOLD57440_830 | Contig623028 | AAFC01623000 | A/G | 285 |
| 350574 | Null | SCAFFOLD95294_1331 | Contig237582 | AAFC01237575 | A/G | 286 |
| 350575 | Null | SCAFFOLD95406_25218 | Contig146950 | AAFC01146944 | A/G | 287 |
| 350825 | Null | SCAFFOLD190005_17363 | Contig618387 | AAFC01618359 | A/G | 288 |
| 350910 | Null | SCAFFOLD231448_1251 | Contig295656 | AAFC01295646 | C/G | 289 |
| 350939 | Null | SCAFFOLD246302_658 | Contig168140 | AAFC01168134 | C/T | 290 |
| 350947 | Null | SCAFFOLD2481_1679 | Contig499892 | AAFC01499876 | A/G | 291 |
| 351213 | Null | SCAFFOLD57440_1077 | Contig623028 | AAFC01623000 | A/G | 292 |
| 351244 | Null | SCAFFOLD70184_5754 | Contig436401 | AAFC01436387 | C/T | 293 |
| 351492 | Null | SCAFFOLD180438_11374 | Contig6788 | AAFC01006788 | A/C | 294 |
| 351586 | Null | SCAFFOLD228449_857 | Contig590514 | AAFC01590490 | A/T | 295 |
| 351608 | Null | SCAFFOLD241780_414 | Contig224973 | AAFC01224967 | A/G | 296 |
| 351679 | Null | SCAFFOLD271779_2717 | Contig64086 | AAFC01064085 | A/C | 297 |
| 351771 | Null | SCAFFOLD313482_2366 | Contig571989 | AAFC01571967 | C/T | 298 |
| 351811 | Null | SCAFFOLD35711_6338 | Contig354565 | AAFC01354555 | A/G | 299 |
| 352000 | Null | SCAFFOLD120053_24927 | Contig192565 | AAFC01192559 | A/G | 300 |
| 352119 | Null | SCAFFOLD175748_9339 | Contig30083 | AAFC01030083 | C/T | 301 |
| 352213 | Null | SCAFFOLD231881_801 | Contig110009 | AAFC01110006 | C/G | 302 |
| 352223 | Null | SCAFFOLD240106_868 | Contig188411 | AAFC01188405 | C/T | 303 |
| 352287 | Null | SCAFFOLD275009_42406 | Contig97781 | AAFC01097779 | C/T | 304 |
| 352332 | Null | SCAFFOLD297961_1269 | Contig426075 | AAFC01426062 | A/C | 305 |
| 352361 | Null | SCAFFOLD314595_482 | Contig474267 | AAFC01474251 | G/T | 306 |
| 352384 | Null | SCAFFOLD359101_308 | Contig643326 | AAFC01643298 | A/G | 307 |
| 352386 | Null | SCAFFOLD36540_3105 | Contig530720 | AAFC01530699 | C/T | 308 |
| 352521 | Null | SCAFFOLD10136_11963 | Contig370580 | AAFC01370570 | C/T | 309 |
| 352656 | Null | SCAFFOLD175694_9476 | Contig147435 | AAFC01147429 | A/G | 310 |
| 352674 | Null | SCAFFOLD185498_17443 | Contig233272 | AAFC01233266 | C/T | 311 |
| 352680 | Null | SCAFFOLD190005_17208 | Contig618387 | AAFC01618359 | A/T | 312 |
| 352880 | Null | SCAFFOLD312013_2996 | Contig695493 | AAFC01695460 | A/G | 313 |
| 352931 | Null | SCAFFOLD5437_10749 | Contig620684 | AAFC01620656 | C/T | 314 |
| 353046 | Null | SCAFFOLD111784_3403 | Contig267069 | AAFC01267060 | A/T | 315 |
| 353087 | Null | SCAFFOLD145449_8789 | Contig125043 | AAFC01125038 | A/T | 316 |
| 353107 | Null | SCAFFOLD155567_7798 | Contig352746 | AAFC01352736 | A/T | 317 |
| 353169 | Null | SCAFFOLD200171_10802 | Contig119374 | AAFC01119370 | C/T | 318 |
| 353195 | Null | SCAFFOLD228449_804 | Contig590514 | AAFC01590490 | C/T | 319 |
| 353436 | Null | SCAFFOLD105396_111 | Contig592585 | AAFC01592560 | C/T | 320 |
| 353461 | Null | SCAFFOLD129380_654 | Contig570695 | AAFC01570673 | G/T | 321 |
| 353547 | Null | SCAFFOLD210324_16928 | Contig271155 | AAFC01271146 | A/C | 322 |
| 353584 | Null | SCAFFOLD25129_31576 | Contig459226 | AAFC01459211 | A/G | 323 |
| 353680 | Null | SCAFFOLD60869_3029 | Contig78108 | AAFC01078107 | A/G | 324 |
| 353897 | Null | SCAFFOLD3764_2424 | Contig378994 | AAFC01378984 | C/G | 325 |
| 353963 | Null | SCAFFOLD101186_2768 | Contig511484 | AAFC01511465 | A/C | 326 |
| 354030 | Null | SCAFFOLD252807_1837 | Contig353800 | AAFC01353790 | C/T | 327 |
| 354084 | Null | SCAFFOLD56872_6443 | Contig539685 | AAFC01539664 | A/G | 328 |
| 354170 | Null | SCAFFOLD256846_676 | Contig450844 | AAFC01450830 | A/T | 329 |
| 354324 | Null | SCAFFOLD41844_3489 | Contig585945 | AAFC01585921 | C/T | 330 |
| 354350 | Null | SCAFFOLD156114_6977 | Contig190583 | AAFC01190577 | G/T | 331 |
| 354364 | Null | SCAFFOLD20725_8663 | Contig703990 | AAFC01703955 | A/T | 332 |
| 354499 | Null | SCAFFOLD286022_2896 | Contig353540 | AAFC01353530 | A/C | 333 |
| 345544 | Null | SCAFFOLD171748_1363 | Contig32037 | AAFC01032036 | A/G | 334 |

TABLE 12
SNP associated with IMF (nir fat) obtained from the IBISS4 database.

| | | | |
|---|---|---|---|
| IBISS4snp344 | IBISS4 | btcn22113 11/17 | 717 |

(SEQ ID NO: 335)

| | |
|---|---|
| TAACTTGCATATTGTGGAACAGCAACATTGTGATTCTCTTGCTCTATTGA | A/G |
| ATGCTTTCCTGTTAATTCTCAATTGAATTGAGCATTCCATGTTTTCTGCT | Homo sapiens OCIA domain containing 2 |
| | (OCIAD2), mRNA 1e−87 |

| | | | |
|---|---|---|---|
| IBISS4snp497 | IBISS4 | btcn26916 8/13 | 724 |

(SEQ ID NO: 336)

| | |
|---|---|
| GAATCGACGGACTACATTTGCCCAATGGAGCCCAGCAACAGTGTTGGTGA | C/T |
| GGTCACAGGGCCTACAGTGGCACCCGGGGCCTCAGCACACTTGATGCCCC | PREDICTED: Homo sapiens PTPRF interacting protein, |
| | binding protein 2 (liprin beta 2) (PPFIBP2), mRNA |
| | 0.0 |

| | | | |
|---|---|---|---|
| IBISS4snp498 | IBISS4 | btcn26916 6/10 | 1258 |

(SEQ ID NO: 337)

| | |
|---|---|
| CCAGAGTTTTGGCTGCAGGGACAGAGCAGGGCCTTCTGCTGTGGGGACAA | C/T |
| GGAACTGTCGTGACTTCATTCAGAGGTGGTCTCTTCTTTCGGTAATAAAA | PREDICTED: Homo sapiens PTPRF interacting protein, |
| | binding protein 2 (liprin beta 2) (PPFIBP2), mRNA |
| | 0.0 |

| | | | |
|---|---|---|---|
| IBISS4snp588 | IBISS4 | btcn28905 11/19 | 851 |

(SEQ ID NO: 338)

| | |
|---|---|
| GGAAGCATTCCTGGCAAAAATGCAGCTGAGTATGAGGTGATCATTGTGAT | T/C GAGCCTGGGCTGCTTTTTGAGA |
| | Homo sapiens mitochondrial isoleucine tRNA |
| | synthetase (FLJ10326), mRNA 0.0 |

| | | | |
|---|---|---|---|
| IBISS4snp589 | IBISS4 | btcn28905 12/23 | 1241 |

(SEQ ID NO: 339)

| | |
|---|---|
| GGCTTTCTGAGCAAGAACCTCCTGACAGGACTGAGTAGGGGTCTAGATGG | G/T |
| TTGTTTACAGTACTGGAGAGAAATCCAAGATTGAGGATGAGTCCAAGTCA | Homo sapiens mitochondrial isoleucine tRNA |
| | synthetase (FLJ10326), mRNA 0.0 |

| | | | |
|---|---|---|---|
| IBISS4snp908 | IBISS4 | btcn50316 10/17 | 1379 |

(SEQ ID NO: 340)

| | |
|---|---|
| GGTTCCAGGGGGGCAGCCCTACCTCACCTTCACCTGTCCCATCCCCCTTC | G/A |
| GTGTGGTGGTGGCCAAAGTGCTCCCAGGGTGCTATACATCAGAGCTGGGC | Homo sapiens FK506 binding protein 10, 65 kDa |
| | (FKBP10), mRNA 0.0 |

| | | | |
|---|---|---|---|
| IBISS4snp1100 | IBISS4 | btcn8874 10/18 | 958 |

(SEQ ID NO: 341)

| | |
|---|---|
| GGACTTCCCCAACCCCTATCGTTTCCCCTGCATCTCGGGGCCCAGGATGC | G/A |
| GTCTGACCTGACCCACACCAAATAGCATTGAGCTGTAAACCTTTTTTTAT | Homo sapiens pituitary tumor-transforming 1 |
| | interacting protein (PTTG1IP), mRNA 3e−21 |

### EXAMPLE 5

To find loci affecting intramuscular fat, further analyses of the whole genome SNP scan were performed. First, the NIRFAT trait values for all measured individuals, not just the 189 in the whole genome scan, were adjusted using the model nirfat ˜ mu herd kill_group age ! sireid using ASREML, in which herd and kill_group are fixed effects, age is a covariate, and sireid is the random effect of sire. The residual NIRFAT phenotypes were then available for further analysis. The means and standard errors of the residual NIRFAT for each genotype in the whole genome scan were calculated and at test computed for each comparison. The statistical significance of the largest mean difference was calculated using 100,000 permutations.

The results of this analysis indicates loci of interest at (P<0.001) (Table 13).

TABLE 13
A set of loci with high levels of support associated with high intramuscular fat.
Snp_id is the ParAllele SNP identifier, scaffold-v2 is the version 2 scaffold, bp is the
location of the SNP in the scaffold, scaffold-v1/IBISS4 gives a direct reference to the SNP,
Hsa is the human chromosome associated with the SNP and Gene is the closest gene to the SNP.

| | scaffold- | | | | | |
|---|---|---|---|---|---|---|
| snp_id | v2 | location | IBISS4/scaffold-v1 | Hsa | Gene | SEQ ID NO: |
| 343800 | 29.18 | 507265 | IBISS4snp713 | 11 | HSPC138 hypothetic | 342 |
| | | | | | protein | |
| 344648 | 4.136 | 45307 | SCAFFOLD146712_2778 | 7 | LOC154790 hypothetic | 343 |
| | | | | | protein | |
| 345273 | 15.136 | 87593 | IBISS4snp894 | 11 | NDUFS3 NADH- | 344 |
| | | | | | ubiquinone | |
| | | | | | oxidoreductase Fe—S | |
| | | | | | protein 3 | |
| 346887 | Un.518 | 242898 | SCAFFOLD4115_5839 | 9 | no gene near | 345 |
| 347425 | 25.88 | 445813 | SCAFFOLD216862_6835 | 7 | PMS2 postmeiotic | 346 |
| | | | | | segregation | |
| | | | | | increased, S. cerevisiae | |
| 349876 | Un.281 | 248625 | SCAFFOLD135077_3922 | X | no gene near | 347 |
| 351544 | Un.16047 | 5152 | SCAFFOLD205589_8358 | | no gene near | 348 |
| 352303 | Un.516 | 289156 | SCAFFOLD285028_33704 | X | near TMEM47 | 349 |
| | | | | | transmembrane protein | |
| | | | | | 47 | |
| 354161 | Un.3082 | 36240 | SCAFFOLD220007_32349 | 4 | no gene near | 350 |
| 354162 | Un.3082 | 36175 | SCAFFOLD220007_32284 | 4 | no gene near | 351 |

TABLE 14
Associations between DNA markers and intramuscular fat sorted in decreasing order of
statistical significance. Locus is the ParAllele identifiers of the polymorphisms, N is the
number of genotypes, mean_0 is the mean net feed intake for genotype 0, mean_1 is the mean for
genotype 1, and mean_2 is the mean of genotype 2, SE is the standard error, a is the additive
effect, k is the dominance effect, alpha is the average effect of allele substitutions, tmax
is the value of the t test, and PermP is the P value determined from 100,000 permutation tests
expressed as a positive integer. A minus value for a means the mean of genotype 0 is lower
than the mean of genotype 2, and a minus value for alpha means that selecting for allele 0
will reduce the average values of intramuscular fat in the population. Scaffold-v2 is the
Draft version 2 Bovine genome sequence, bp is the location in base pairs in the scaffold, and
bases are the alternative bases.

| snp_id | scaffold-v2 | bp | bases | | mean0 | SE | mean1 | SE | mean2 | SE |
|---|---|---|---|---|---|---|---|---|---|---|
| 344648 | 4.136 | 45307 | G/T | 344648 | 1.90 | 0.61 | −0.28 | 0.21 | −0.16 | 0.11 |
| 346887 | Un.518 | 242898 | C/T | 346887 | −0.26 | 0.11 | 0.84 | 0.29 | 0.27 | 1.14 |
| 343800 | 29.18 | 507265 | A/G | 343800 | −0.21 | 0.21 | 0.23 | 0.16 | −0.46 | 0.17 |
| 345273 | 15.136 | 87593 | C/T | 345273 | −0.50 | 0.13 | 0.24 | 0.16 | 0.13 | 0.31 |
| 351544 | Un.16047 | 5152 | C/T | 351544 | −0.06 | 0.11 | −0.53 | 0.24 | 1.38 | 0.43 |
| 349876 | Un.281 | 248625 | C/T | 349876 | −0.12 | 0.11 | −0.22 | 0.22 | 1.64 | 0.84 |
| 352303 | Un.516 | 289156 | C/T | 352303 | 0.26 | 0.14 | NaN | NaN | −0.40 | 0.14 |
| 347425 | 25.88 | 445813 | A/G | 347425 | −0.48 | 0.18 | 0.41 | 0.19 | −0.23 | 0.14 |
| 354161 | Un.3082 | 36240 | C/T | 354161 | −0.01 | 0.14 | −0.43 | 0.15 | 0.63 | 0.36 |
| 354162 | Un.3082 | 36175 | G/T | 354162 | 1.73 | 0.79 | −0.44 | 0.16 | −0.01 | 0.13 |

| snp_id | N | Freq | a | −k | alpha | tmax | PermP | SEQ ID NO: |
|---|---|---|---|---|---|---|---|---|
| 344648 | 162 | 0.18 | 1.03 | −1.12 | 1.76 | 4.67 | 0.0000 | 343 |
| 346887 | 162 | 0.90 | −0.26 | −3.18 | 0.41 | 4.04 | 0.0000 | 345 |
| 343800 | 155 | 0.45 | 0.12 | 4.55 | 0.06 | 2.97 | 0.0001 | 342 |
| 345273 | 149 | 0.62 | −0.31 | −1.36 | −0.21 | 3.59 | 0.0001 | 344 |
| 351544 | 163 | 0.84 | −0.72 | 1.64 | −1.52 | 3.64 | 0.0002 | 348 |
| 349876 | 163 | 0.82 | −0.88 | 1.12 | −1.50 | 3.59 | 0.0003 | 347 |
| 352303 | 163 | 0.48 | 0.33 | NaN | NaN | 3.31 | 0.0006 | 349 |
| 347425 | 162 | 0.50 | −0.13 | −6.12 | −0.12 | 3.41 | 0.0007 | 346 |
| 354161 | 163 | 0.73 | −0.32 | 2.28 | −0.66 | 3.22 | 0.0008 | 350 |
| 354162 | 162 | 0.19 | 0.87 | −1.50 | 1.68 | 4.19 | 0.0008 | 351 |

TABLE 15

DNA sequences for each of the SNP ID in
Table 13.

| | | | |
|---|---|---|---|
| | | | 3′ |
| | scaffold- | 5′flanking sequence | flanking |
| Snp_id | v1/IBISS4 | alternative bases | sequence |

(SEQ ID NO: 342)

| | | | |
|---|---|---|---|
| 343800 | IBISS4snp713 | GCGTTCCTGCCCCTGTCCT | A/G |
| | | CGTCACTATGTAGCTGAGG | |
| | | GGCAGGAGGCCTTTCCACG | |
| | | TTCTGGAAGGTTCTTGGGC | |
| | | TCGACCACGGCAGTAGCCC | |
| | | CAGGA | |

(SEQ ID NO: 343)

| | | | |
|---|---|---|---|
| 344648 | SCAFFOLD146712_ | TCAGAAGGGAAAGAAAAAT | T/G |
| | 2778 | GCATGTGTTTAGTCTGCCG | |
| | | TCTTTATTTGCAAGTCCAA | |
| | | CAGACATTCGGGAGTCCTT | |
| | | GCTATGGGCATGGTTTGTA | |
| | | CTGACAACTAAGCAGGATC | |
| | | CAGAGGCGGGCGGCCCCAG | |
| | | GCCCGTTAAGAGCAGAGGG | |
| | | CTGAACCCCATCTGGGTCA | |
| | | GCTCTGCCGTCCCCAGGGG | |
| | | CTGCGTGTTGTAAGGTTGA | |
| | | TGCCCTTGAGCTGCATGGT | |
| | | GGCGCCCATGGCAAGTACG | |
| | | CTG | |
| | | GTGAATCCTTCTTTTTCCG | |
| | | TCCATAAACACCTCTCAGT | |
| | | CCGCATTGTAAACCACTTC | |
| | | ACCTGGGTTCACATTCCAG | |
| | | CTCTGCCACTTTCTTGTGG | |
| | | CAAAGTTACTTAGCTTCTA | |
| | | TTTCATTTTCCTTGTCTTT | |
| | | AAGACAAAGTCATTATAGT | |
| | | ATCTGCCTCTTAGAATTTT | |
| | | ATGAGAATTCAACAAATTG | |
| | | TGCTTAGAGCAGTGCCTGG | |
| | | CACAGTAAGCTCTCCTTCA | |
| | | GTGTCAGCAACTACCATCA | |
| | | TGA | |

(SEQ ID NO: 344)

| | | | |
|---|---|---|---|
| 345273 | IBISS4snp894 | CATCCTGATGGGGTCATCC | T/C |
| | | CAGTGCTGACTTTCCTCAG | |
| | | GGATCACAGCAAGCACAAT | |
| | | TCAAATCCTTGGCTGACTT | |
| | | GA | |

(SEQ ID NO: 345)

| | | | |
|---|---|---|---|
| 346887 | SCAFFOLD4115_ | GCTTTCTGCCTCAGTGGGA | C/T |
| | 5839 | GACACACCAAAATCAGTGC | |
| | | CCCTTGGACTGTGATCTGT | |
| | | GCTGTGCTGTGCTTAGTTG | |
| | | CTCTGTCGTGTCCAACCCT | |
| | | TTGTGACCCTATGGACTGT | |
| | | AACCTGCCAGGCTTCTCTG | |
| | | TCCATGGGGATTCTCCAGG | |
| | | CAAGAGTACTGGAGTGGGC | |
| | | AGCCATGCCCTCCTCCAGG | |
| | | GGATCTTCCCCCTGGATCT | |
| | | TCCCAACCCAGGTCTCAGG | |
| | | AATATCACTTACCCTCACT | |
| | | GGG | |
| | | CTCACTATCCTTATCTCTG | |
| | | ACATGATGGGACTGGAACT | |
| | | ATTGACCTCTAAGATCTCT | |
| | | CTTCAGCTCTAACATGCTC | |
| | | TGGTTATAAGCACCTTTGG | |
| | | ATGACGGCAATATTTATAG | |
| | | GCAATGATATGACTAGAGA | |
| | | ATATTTAGACTGATTCATA | |
| | | GTCTGTGTGAGGCAGAGAA | |
| | | GAACATGCAAAGTCTTTTG | |
| | | GGAAAGAAGTTATTTTTGA | |
| | | GTGCTGTAAAACCCTAGTA | |
| | | ATAAAACACAAATTCAAAA | |
| | | ATG | |

(SEQ ID NO: 346)

| | | | |
|---|---|---|---|
| 347425 | SCAFFOLD216862_ | TCTGCTCAGGGGCCCCACG | G/A |
| | 6835 | ACTTGATGAGCAGAGTGGA | |
| | | GATGGAGGAGGACTCGGGG | |
| | | CACGGCAGCGCATCTGCCG | |
| | | GCTCCGAGGAGGCATCCAG | |
| | | CACCCCCGAGACAAGCAGT | |
| | | CACCCCAGCACTGACCGGG | |
| | | TGGCAAGCTCCCCCGAAGA | |
| | | CAAATTTTCCCAAGAAAAT | |
| | | ATGGAGTCTCGGGAAAAGT | |
| | | TACCTGAAACTGACCATCG | |
| | | TCTCTCAGGCACAAAGTGC | |
| | | CATTTAAACCAAGAAGAAA | |
| | | GCA | |
| | | CTCCACATCTGGAGTCTTG | |
| | | CCGCAGCCCACAAAGCTCT | |
| | | CATCCCCAAATGCAAAGCG | |
| | | TTTTACAAAAGAAGGGATT | |
| | | CCTTTAAATCCCAGTGTCC | |
| | | TTGCAGAGTCGGTGAAAGC | |
| | | CCAGAGTGCGTCAGCTACA | |
| | | GTGGATGTAGCTGTGAAAA | |
| | | TTAACAAGAAAATAGTGCC | |
| | | CCTTGATTTTTCCATGAGT | |
| | | TCTTTAGCCAAACGAATAA | |
| | | AGCAGTTGTGTCAGCAAGA | |
| | | ACAGCAACAAGAAAGTCAA | |
| | | CAG | |

(SEQ ID NO: 347)

| | | | |
|---|---|---|---|
| 349876 | SCAFFOLD135077_ | AGACTCAGATATCCTGCAT | T/C |
| | 3922 | TGGCATGTGGATTCCTAAC | |
| | | CACTGGACCACCAACCAGG | |
| | | AAACCCTCACGTTATGTTC | |
| | | CTATTAACACCTCCATCGA | |
| | | CTTCTCATCCCTCTTGTCT | |
| | | GTTCCTTTAGTCAAGCTGG | |
| | | CTGCCCACATCGCATTGTT | |
| | | TTAATTACTATAAACCTTC | |
| | | TAATGTGATGTATCCTGTT | |
| | | GTAATGCTGTTTAGTAGGA | |
| | | GAATTGCCCCCAACTTTTT | |
| | | ATTATCCTTCCAAATAGTG | |
| | | TTGG | |
| | | AGTTCTTATACACTCACTG | |
| | | TCCATGTGAAAAAAATCAC | |
| | | CATCGTTTGCAGTGTAAAG | |
| | | AAATACGCTATCGTGGTTA | |
| | | TGCTAGCTGATTTGCATTG | |
| | | AACTGATGGCTTACTCCAA | |
| | | GGATAATAAACATCTTTAA | |
| | | AGATCATTGCGTCTTCCCT | |
| | | TTAGTGGGAAGTGAAAGTT | |
| | | GTCCAACTCTTTGCGACAC | |
| | | CATGGACTGTCCATGGAAT | |
| | | TCTTCAGGCCAGAATACTG | |
| | | GAGTGGGTATCCTTGCCCT | |
| | | TCC | |

(SEQ ID NO: 348)

| | | | |
|---|---|---|---|
| 351544 | SCAFFOLD205589_ | AGCCCACCAGGCTCCCCTG | T/C |
| | 8358 | TCCCTGGGATTCTCCAGGC | |
| | | AAGAACACTGGAGCGGGTT | |
| | | GCCATTTCCTTCTCCCATG | |
| | | TATGAAAGTGAAAACTCAA | |
| | | AGTGAAGTCGCTGAGTCAT | |
| | | GTCCGCCTGTTAGTGACCC | |
| | | ATGGACTGCAACCTACCAG | |
| | | GCTCCTCCATCCATGGGAT | |
| | | TTTCCAGGCAAGAGTACTG | |
| | | GAGTGGATTGTCATTTCCT | |
| | | TCTCCATTTTTTAGTTCTA | |
| | | TGGAAACACAAAAGACTTT | |
| | | GAA | |
| | | ACAAAACACAAAAGTCTTT | |
| | | GAAAAGACACAGTAATCTT | |
| | | GAGAAATAACAGAGCTGGA | |
| | | GTAGTCATACTCCCAGATT | |
| | | TTATACTATATTACAAAGC | |
| | | TACAGTAATCTAGACAGTA | |
| | | TGGTAGTGGCACAAAAGCA | |
| | | GAAATATAGATCAATGGAA | |
| | | CAGCATAGAAAGTCCAGAG | |
| | | ACACATCCATGCACCTACG | |
| | | GTCAATTAATCCTACAACA | |
| | | AAGGAGGCTAGAATATAAA | |
| | | AGAATAGAATGGAAAAGAC | |
| | | AGTC | |

(SEQ ID NO: 349)

| | | | |
|---|---|---|---|
| 352303 | SCAFFOLD285028_ | ATTGGTCAGTTTCATGCAG | T/C |
| | 33704 | GTCTGAATATACACATTTT | |
| | | GATTGAATTTGCTCAAAAG | |
| | | AAGATAGCTGCACTGTTAA | |
| | | TCATATTTATTAGTTGATC | |
| | | TTAAGAATTCATGCATTTA | |
| | | ATTGTTCTAGAATTAGAGA | |
| | | TAAACCTTGGAATTATGAC | |
| | | TGTGAGAAATGGCCAAGAG | |
| | | TTTCATTGGGGATGACAGA | |
| | | AGGCAATGCTGAGCAAGAA | |
| | | GGAGGTTTGAAATCCTGCA | |
| | | TTAATTCTAGAGTTCATCA | |
| | | ACC | |
| | | ATGACTATGGAAATCTCGA | |
| | | TATATTTGAATTGCAAAAA | |
| | | TTTATAAAAAACTCTTAAA | |
| | | TACTTTTTGTCCTTCTGTT | |
| | | TTCTCCTTCAATATAAGCC | |
| | | TACAAAATTATAAGGTACT | |
| | | TAAGTGTTGGGACCATGTC | |
| | | TCTGAATCTTTAATAACAA | |
| | | ACACACTCTCATAGTAGGT | |
| | | GTCCAAAACTATATTATTG | |
| | | ATTGTTTTATATGTGAGTT | |
| | | GGAGAAAAGATGCATAAAA | |
| | | ATGTCTAAGCATGCTAGAA | |
| | | TTCT | |

(SEQ ID NO: 350)

| | | | |
|---|---|---|---|
| 354161 | SCAFFOLD220007_ | TAATTAGGATACTGTTCCA | C/T |
| | 32349 | AGAAATTGGATACATATTA | |
| | | CTGTGTACCAAATATTATA | |
| | | GTCAGTGCTGATGTCAGGA | |
| | | TCACTAGTGAATTAAGAGT | |
| | | CAAATGGAGAAACAGACAT | |
| | | GGAAACGGGAATGCAGACT | |
| | | ATGATGTAACAAAGAACAT | |
| | | CTGGATAAAGCTGAAAGAA | |
| | | GGTCATACAAGATTAACAG | |
| | | TGGTGCCAGGTCTGACGCA | |
| | | TTGGAGGCAAGTGACAAAT | |
| | | ATCTAATAGGTGAATGAAT | |
| | | GAA | |
| | | AACTCATAAATGATTTTTT | |
| | | GAAATTTCTTTTAAAAATG | |
| | | AGTTTGATTTTAATTTACT | |
| | | AATCAATTAAAGATAGAAT | |
| | | TTATGGGAAAATGGAAAAC | |
| | | TCTGGCAGGGTCACAGTTT | |
| | | GTAGACTGATACAAAAGGA | |
| | | GATATATTTCTATCAATGA | |
| | | AATTGTTTGATACAATCAG | |
| | | GACACTTTAAGATCCTTAA | |
| | | AAATTATTCAGAGCCCAAA | |
| | | AGAGCTTTTATTTTTGTAG | |
| | | GCTAAAGTCCTTAACATTT | |
| | | AC | |

(SEQ ID NO: 351)

| | | | |
|---|---|---|---|
| 354162 | SCAFFOLD220007_ | TGATGTCAGGATCACTAGT | T/G |
| | 32284 | GAATTAAGAGTCAAATGGA | |
| | | GAAACAGACATGGAAACGG | |
| | | GAATGCAGACTATGATGTA | |
| | | ACAAAGAACATCTGGATAA | |
| | | AGCTGAAAGAAGGTCATAC | |
| | | AAGATTAACAGTGGTGCCA | |
| | | GGTCTGACGCATTGGAGGC | |
| | | AAGTGACAAATATCTAATA | |
| | | GGTGAATGAATGAACAACT | |
| | | CATAAAATGATTTTTTGAA | |
| | | ATTTCTTTTAAAAATGAGT | |
| | | TTGATTTTAATTTACTAAT | |
| | | CAA | |
| | | TAAAGATAGAATTTATGGG | |
| | | AAAATGGAAAACTCTGGCA | |
| | | GGGTCACAGTTTGTAGACT | |
| | | GATACAAAAGGAGATATAT | |
| | | TTCTATCAATGAAATTGTT | |
| | | TGATACAATCAGGACACTT | |
| | | TAAGATCCTTAAAAATTAT | |
| | | TCAGAGCCCAAAAGAGCTT | |
| | | TTATTTTTGTAGGCTAAAG | |
| | | TCCTTAACATTTACCTTCT | |
| | | TAGAAATTCAAACTAAGAA | |
| | | CTCTAAAAAGATATTTATT | |
| | | CATTTTAAACAAAAATAAA | |
| | | CTCA | |

TABLE 16
A further set of loci associated with high intramuscular fat. Snp_id is the ParAllele
SNP identifier, scaffold-v2 is the version 2 scaffold, bp is the location of the SNP in the
scaffold, bases is the alternative bases in the SNP, mean0 is the mean of the genotype higher up
in the with the alphabet, SE is the standard error of the mean, N is the total sample size, Freq
is the frequency of the 0 allele, a is half the distance between the homozygotes, k is the
dominance effect, a is the average effect of allele substitution in residual RBY, tmax is the t
test between the genotypes with the biggest difference in residual RBY and PermP is the P value
resulting from of 100,000 permutations of the data. These may be correlated with the DNA
sequence (and SEQ ID NO:) by reference to Table 17, where the SNP identification numbers (first
column in Table 17) are listed in ascending, numerical order.

| snp_id | scaffold-v2 | bp | bases | | mean0 | SE | mean1 | SE | mean2 | SE |
|---|---|---|---|---|---|---|---|---|---|---|
| 352783 | Un.637 | 282951 | C/T | 352783 | 0.11 | 0.14 | −0.58 | 0.15 | −0.06 | 0.29 |
| 350590 | 27.26 | 213231 | C/T | 350590 | 0.11 | 0.14 | −0.07 | 0.18 | −0.75 | 0.19 |
| 347369 | Un.8032 | 21710 | C/T | 347369 | 0.20 | 0.22 | −0.50 | 0.12 | 0.08 | 0.33 |
| 349456 | Un.516 | 288740 | G/T | 349456 | 0.28 | 0.14 | NaN | NaN | −0.40 | 0.14 |
| 349616 | 13.44 | 87079 | C/T | 349616 | −0.25 | 0.11 | 0.62 | 0.27 | −0.87 | 0.27 |
| 351687 | Un.5261 | 28632 | A/G | 351687 | 0.83 | 0.41 | −0.37 | 0.14 | −0.02 | 0.14 |
| 353318 | 28.47 | 47391 | A/C | 353318 | −0.32 | 0.15 | 0.49 | 0.21 | −0.51 | 0.23 |
| 345109 | Un.3135 | 56803 | A/G | 345109 | −0.15 | 0.11 | −0.11 | 0.30 | 1.36 | 0.81 |
| 350915 | Un.528 | 414390 | A/G | 350915 | −0.06 | 0.17 | −0.37 | 0.13 | 0.56 | 0.28 |
| 343916 | Un.10004 | 12767 | A/G | 343916 | −0.08 | 0.13 | −0.17 | 0.15 | 1.85 | 0.80 |
| 351993 | Un.1858 | 13608 | C/T | 351993 | −1.15 | 0.26 | −0.05 | 0.18 | 0.02 | 0.12 |
| 352221 | Un.4383 | 55920 | A/T | 352221 | −0.08 | 0.13 | −0.31 | 0.16 | 0.72 | 0.38 |
| 353634 | Un.932 | 29516 | C/T | 353634 | 0.04 | 0.12 | −0.08 | 0.21 | −1.15 | 0.38 |
| 345655 | 25.76 | 437300 | G/T | 345655 | 0.32 | 0.35 | 0.22 | 0.18 | −0.47 | 0.12 |
| 347176 | 1.128 | 533301 | A/G | 347176 | 0.21 | 0.15 | −0.54 | 0.16 | −0.14 | 0.21 |
| 352760 | Un.2361 | 101930 | A/G | 352760 | 1.33 | 0.83 | −0.38 | 0.15 | −0.01 | 0.13 |
| 348968 | Un.156 | 58421 | A/C | 348968 | −0.07 | 0.12 | −0.34 | 0.19 | 2.02 | 0.44 |
| 345554 | Un.4168 | 46080 | C/T | 345554 | −0.06 | 0.62 | −0.61 | 0.14 | 0.11 | 0.13 |
| 342512 | Un.227 | 375024 | C/T | 342512 | −0.12 | 0.12 | −0.24 | 0.21 | 1.24 | 0.49 |
| 344894 | Un.2361 | 102194 | C/G | 344894 | −0.00 | 0.13 | −0.39 | 0.15 | 1.33 | 0.83 |
| 353039 | Un.11559 | 12577 | A/C | 353039 | −0.03 | 0.26 | −0.39 | 0.13 | 0.33 | 0.23 |
| 353800 | 17.16 | 123090 | A/T | 353800 | 2.08 | 0.82 | 0.42 | 0.23 | −0.26 | 0.11 |
| 354182 | 4.151 | 1228627 | C/T | 354182 | −0.31 | 0.11 | 0.35 | 0.21 | 0.28 | 0.60 |
| 348929 | 7.66 | 112309 | C/T | 348929 | −0.04 | 0.12 | −0.52 | 0.19 | 1.12 | 0.63 |
| 354184 | Un.516 | 289120 | A/C | 354184 | −0.40 | 0.14 | NaN | NaN | 0.26 | 0.14 |
| 354414 | 3.83 | 50176 | C/T | 354414 | −0.05 | 0.26 | −0.37 | 0.13 | 0.33 | 0.20 |
| 346388 | Un.17378 | 2907 | A/G | 346388 | −0.06 | 0.30 | −0.48 | 0.15 | 0.14 | 0.15 |
| 349564 | 9.37 | 408918 | A/G | 349564 | −0.79 | 0.22 | −0.06 | 0.15 | 0.17 | 0.16 |
| 351131 | 17.2 | 175491 | A/C | 351131 | −0.16 | 0.10 | 1.32 | 0.80 | NaN | NaN |
| 347617 | 12.46 | 349451 | C/T | 347617 | 0.76 | 0.41 | −0.28 | 0.14 | −0.09 | 0.15 |
| 347981 | 1.93 | 40166 | C/T | 347981 | 0.04 | 0.17 | 0.07 | 0.16 | −0.72 | 0.21 |
| 344620 | 30.26 | 11716 | G/T | 344620 | −0.01 | 0.12 | −0.68 | 0.23 | 0.11 | 0.68 |
| 344645 | Un.518 | 312470 | A/G | 344645 | −0.06 | 0.53 | 0.48 | 0.22 | −0.23 | 0.12 |
| 343505 | 5.166 | 168656 | A/G | 343505 | 0.87 | 0.48 | −0.44 | 0.20 | −0.09 | 0.12 |
| 348415 | 10.153 | 1965179 | A/G | 348415 | 1.10 | 0.56 | −0.07 | 0.16 | −0.23 | 0.13 |
| 343071 | Un.306 | 264740 | A/G | 343071 | 1.17 | 0.54 | −0.02 | 0.51 | −0.21 | 0.10 |
| 343076 | 9.129 | 93921 | A/G | 343076 | 0.03 | 0.19 | 0.22 | 0.20 | −0.53 | 0.16 |
| 353926 | NULL | NULL | C/T | 353926 | 0.02 | 0.11 | −1.18 | 0.28 | 0.15 | 0.23 |
| 348572 | Un.516 | 289241 | C/T | 348572 | −0.39 | 0.14 | NaN | NaN | 0.23 | 0.14 |
| 353896 | Un.25 | 701897 | C/T | 353896 | 1.18 | 0.78 | 0.05 | 0.27 | −0.19 | 0.10 |
| 348185 | 2.162 | 167169 | C/G | 348185 | −0.17 | 0.12 | −0.14 | 0.20 | 1.02 | 0.37 |
| 352138 | 2.96 | 311148 | C/G | 352138 | −0.01 | 0.12 | −0.52 | 0.16 | 0.94 | 0.72 |
| 345838 | 24.58 | 150701 | A/G | 345838 | −0.28 | 0.13 | 0.34 | 0.19 | −0.23 | 0.44 |
| 346372 | 25.91 | 858266 | A/G | 346372 | −0.18 | 0.12 | −0.22 | 0.18 | 0.73 | 0.35 |
| 347168 | 13.53 | 215094 | A/G | 347168 | −0.38 | 0.15 | 0.37 | 0.22 | −0.23 | 0.16 |
| 342743 | 19.45 | 442850 | C/T | 342743 | −0.03 | 0.14 | 0.09 | 0.20 | −1.00 | 0.21 |
| 354282 | Un.2920 | 36226 | C/T | 354282 | 0.04 | 0.17 | 0.11 | 0.17 | −0.55 | 0.18 |
| 342778 | 22.85 | 150222 | C/T | 342778 | −0.24 | 0.11 | 0.52 | 0.41 | 1.12 | 0.62 |
| 342709 | Un.4773 | 38302 | C/T | 342709 | −0.26 | 0.11 | 0.40 | 0.25 | −0.14 | 0.42 |
| 353681 | Un.11581 | 11098 | C/T | 353681 | 0.07 | 0.13 | −0.54 | 0.16 | 0.40 | 0.38 |
| 348459 | Un.6824 | 3277 | A/G | 348459 | 0.03 | 0.21 | −0.40 | 0.14 | 0.28 | 0.20 |
| 351663 | Un.6724 | 20355 | C/T | 351663 | 0.06 | 0.22 | −0.40 | 0.14 | 0.24 | 0.18 |
| 353643 | 3.19 | 93615 | C/G | 353643 | −0.10 | 0.18 | −0.30 | 0.15 | 0.36 | 0.21 |
| 351089 | 27.3 | 318658 | C/G | 351089 | −0.69 | 0.22 | 0.07 | 0.16 | 0.04 | 0.16 |
| 344880 | Un.9044 | 12820 | C/T | 344880 | −0.02 | 0.30 | −0.35 | 0.13 | 0.22 | 0.18 |
| 350721 | 25.32 | 172400 | C/T | 350721 | 0.16 | 0.14 | −0.24 | 0.17 | −0.71 | 0.28 |
| 353868 | Un.4123 | 3772 | A/G | 353868 | 1.46 | 0.42 | 0.09 | 0.25 | −0.17 | 0.11 |
| 346220 | Un.5571 | 12011 | G/T | 346220 | −0.16 | 0.31 | −1.30 | 0.33 | −0.04 | 0.11 |
| 354055 | Un.789 | 46267 | C/T | 354055 | −0.15 | 0.11 | −0.63 | 0.12 | 0.34 | 0.36 |
| 342518 | 26.7 | 229556 | C/T | 342518 | 0.01 | 0.14 | −0.40 | 0.15 | 0.91 | 0.62 |
| 342896 | Un.3818 | 47739 | A/G | 342896 | −0.32 | 0.16 | 0.46 | 0.24 | −0.06 | 0.21 |
| 344366 | 22.2 | 325125 | C/G | 344366 | 0.07 | 0.50 | 0.36 | 0.22 | −0.27 | 0.12 |
| 348325 | Un.2742 | 92194 | A/G | 348325 | −0.26 | 0.14 | 0.42 | 0.19 | −0.38 | 0.18 |
| 351334 | 5.1 | 1504709 | C/T | 351334 | −0.01 | 0.23 | 0.07 | 0.13 | −0.59 | 0.17 |
| 352556 | Un.5582 | 16606 | A/C | 352556 | 0.26 | 0.23 | 2.18 | 0.87 | −0.23 | 0.12 |
| 352985 | Un.2169 | 113582 | A/G | 352985 | −0.13 | 0.17 | −0.31 | 0.14 | 0.35 | 0.24 |
| 350226 | Un.1554 | 186089 | A/T | 350226 | 0.80 | 0.37 | −0.17 | 0.16 | −0.18 | 0.14 |
| 349583 | 4.96 | 282594 | A/G | 349583 | 0.84 | 0.64 | −0.59 | 0.19 | −0.04 | 0.12 |
| 348246 | 4.8 | 787563 | C/T | 348246 | −0.17 | 0.10 | 0.77 | 0.43 | NaN | NaN |
| 349492 | Un.1441 | 108562 | C/T | 349492 | −0.09 | 0.17 | −0.29 | 0.15 | 0.47 | 0.23 |
| 345395 | Un.5582 | 16471 | A/C | 345395 | −0.18 | 0.17 | −0.28 | 0.14 | 0.43 | 0.25 |
| 349106 | Un.7325 | 25630 | C/T | 349106 | 0.18 | 0.17 | −0.06 | 0.15 | −0.59 | 0.23 |
| 349116 | Un.12818 | 12550 | C/T | 349116 | −0.04 | 0.17 | 0.11 | 0.17 | −0.60 | 0.14 |
| 354174 | 14.43 | 675522 | G/T | 354174 | −0.18 | 0.42 | −0.50 | 0.14 | 0.14 | 0.14 |
| 342524 | Un.10647 | 15369 | C/T | 342524 | −0.16 | 0.12 | −0.26 | 0.23 | 0.68 | 0.36 |
| 349299 | Un.434 | 223954 | A/C | 349299 | −0.46 | 0.17 | 0.25 | 0.17 | −0.20 | 0.16 |
| 353752 | Un.5582 | 16662 | C/T | 353752 | −0.18 | 0.17 | −0.28 | 0.14 | 0.43 | 0.25 |
| 343971 | Un.1944 | 36477 | C/T | 343971 | −0.17 | 0.12 | −0.16 | 0.17 | 1.00 | 0.58 |
| 346305 | 28.4 | 29606 | A/C | 346305 | −0.27 | 0.18 | −0.47 | 0.13 | 0.16 | 0.16 |
| 353054 | 15.86 | 331848 | A/G | 353054 | 2.01 | 0.59 | −0.48 | 0.25 | −0.05 | 0.11 |
| 353060 | Un.5582 | 16876 | C/T | 353060 | 0.43 | 0.25 | −0.28 | 0.14 | −0.18 | 0.17 |
| 343405 | 18.146 | 566661 | A/G | 343405 | −0.28 | 0.28 | 0.20 | 0.18 | −0.45 | 0.17 |
| 346429 | Un.12818 | 12728 | C/T | 346429 | −0.04 | 0.17 | 0.10 | 0.16 | −0.60 | 0.14 |
| 348275 | Un.1926 | 37849 | G/T | 348275 | 0.15 | 0.17 | −0.48 | 0.15 | 0.20 | 0.21 |
| 345839 | 15.128 | 6678 | A/G | 345839 | 0.13 | 0.14 | −0.53 | 0.14 | −0.31 | 0.30 |
| 351743 | Un.789 | 45942 | A/C | 351743 | −0.13 | 0.15 | −0.26 | 0.15 | 0.58 | 0.31 |
| 345160 | 17.77 | 230082 | A/C | 345160 | −0.31 | 0.12 | −0.04 | 0.19 | 0.49 | 0.30 |
| 346062 | Un.2716 | 5535 | A/G | 346062 | −0.02 | 0.16 | −0.41 | 0.15 | 0.32 | 0.26 |
| 352701 | 8.52 | 958 | A/C | 352701 | 0.25 | 0.18 | −0.40 | 0.16 | −0.01 | 0.19 |
| 350780 | 28.64 | 36585 | A/G | 350780 | −0.17 | 0.31 | −0.40 | 0.16 | 0.18 | 0.15 |
| 351863 | Un.10682 | 6600 | A/G | 351863 | 2.08 | 1.01 | 0.29 | 0.37 | −0.15 | 0.11 |
| 350519 | Un.11 | 1156244 | C/T | 350519 | 0.23 | 0.16 | −0.33 | 0.15 | −0.32 | 0.30 |
| 345464 | Un.42231 | 1024 | C/T | 345464 | −0.16 | 0.11 | 0.98 | 0.39 | −0.63 | 0.41 |
| 351654 | Un.5066 | 4868 | C/T | 351654 | −0.02 | 0.12 | −0.47 | 0.17 | 1.13 | 0.77 |
| 354135 | Un.7528 | 36561 | A/T | 354135 | 0.29 | 0.25 | 0.06 | 0.18 | −0.34 | 0.12 |
| 347988 | 4.136 | 966787 | G/T | 347988 | −0.37 | 0.14 | 0.19 | 0.17 | 0.03 | 0.33 |
| 348126 | Un.1825 | 148535 | C/T | 348126 | 0.10 | 0.30 | 0.44 | 0.29 | −0.24 | 0.11 |
| 352915 | NULL | NULL | G/T | 352915 | 0.01 | 0.15 | −0.45 | 0.16 | 0.22 | 0.21 |
| 349323 | Un.114 | 438261 | C/G | 349323 | −0.08 | 0.12 | 0.19 | 0.22 | −1.75 | 0.55 |
| 351593 | 3.12 | 84976 | C/T | 351593 | −0.23 | 0.10 | 0.55 | 0.31 | −1.25 | NaN |
| 347802 | 25.5 | 15756 | A/G | 347802 | 0.13 | 0.67 | 0.39 | 0.23 | −0.23 | 0.11 |
| 350996 | NULL | NULL | C/T | 350996 | −0.50 | 0.19 | 0.27 | 0.17 | −0.18 | 0.16 |
| 349235 | Un.4539 | 42294 | A/G | 349235 | 1.89 | 1.19 | 0.11 | 0.30 | −0.17 | 0.11 |
| 351065 | Un.8255 | 16739 | A/G | 351065 | 0.35 | 0.27 | −0.59 | 0.20 | −0.03 | 0.12 |
| 343441 | 5.169 | 996113 | A/G | 343441 | −0.00 | 0.28 | −0.35 | 0.14 | 0.23 | 0.17 |
| 343516 | 29.3 | 130941 | A/G | 343516 | −0.16 | 0.17 | 0.19 | 0.17 | −0.48 | 0.16 |
| 342993 | 17.23 | 446279 | A/G | 342993 | −0.08 | 0.13 | −0.33 | 0.16 | 0.83 | 0.45 |
| 349117 | Un.12818 | 12771 | A/G | 349117 | −0.04 | 0.17 | 0.09 | 0.17 | −0.60 | 0.14 |
| 346498 | 12.66 | 188317 | A/G | 346498 | 0.30 | 0.18 | −0.46 | 0.18 | −0.04 | 0.14 |
| 346312 | 4.69 | 62348 | A/G | 346312 | NaN | NaN | 0.59 | 0.38 | −0.18 | 0.10 |
| 346497 | Un.1794 | 106945 | A/C | 346497 | −0.65 | 0.26 | −0.14 | 0.19 | 0.11 | 0.14 |
| 343880 | Un.2336 | 110695 | C/T | 343880 | −0.01 | 0.13 | −0.53 | 0.19 | 0.45 | 0.36 |
| 345945 | 16.21 | 272163 | C/T | 345945 | −0.43 | 0.14 | 0.16 | 0.17 | −0.07 | 0.24 |
| 345210 | 26.6 | 495904 | C/T | 345210 | −0.39 | 0.12 | 0.20 | 0.19 | −0.14 | 0.25 |
| 354455 | 8.1 | 229378 | C/T | 354455 | 0.20 | 0.59 | −0.51 | 0.19 | 0.06 | 0.12 |
| 350589 | 6.111 | 493692 | A/C | 350589 | −0.22 | 0.11 | −0.07 | 0.21 | 0.62 | 0.48 |
| 351724 | 3.114 | 11398 | A/G | 351724 | −0.14 | 0.13 | 0.27 | 0.20 | −0.77 | 0.32 |

| snp_id | N | Freq | a | −k | alpha | tmax | PermP |
|---|---|---|---|---|---|---|---|
| 352783 | 162 | 0.78 | 0.09 | −7.10 | −0.25 | 2.91 | 0.0009 |
| 350590 | 163 | 0.66 | 0.43 | 0.58 | 0.51 | 3.06 | 0.0010 |
| 347369 | 135 | 0.61 | 0.06 | −11.38 | −0.09 | 2.96 | 0.0011 |
| 349456 | 162 | 0.48 | 0.34 | NaN | NaN | 3.37 | 0.0011 |
| 349616 | 162 | 0.85 | 0.31 | 3.83 | 1.15 | 3.55 | 0.0011 |
| 351687 | 163 | 0.25 | 0.43 | −1.83 | 0.82 | 3.27 | 0.0013 |
| 353318 | 143 | 0.69 | 0.10 | 9.46 | 0.44 | 3.23 | 0.0014 |
| 345109 | 163 | 0.89 | −0.76 | 0.94 | −1.31 | 3.09 | 0.0016 |
| 350915 | 161 | 0.60 | −0.31 | 2.00 | −0.43 | 3.30 | 0.0016 |
| 343916 | 159 | 0.86 | −0.97 | 1.09 | −1.73 | 3.62 | 0.0017 |
| 351993 | 163 | 0.29 | −0.58 | −0.87 | −0.80 | 3.23 | 0.0017 |
| 352221 | 161 | 0.71 | −0.40 | 1.57 | −0.66 | 2.99 | 0.0017 |
| 353634 | 162 | 0.77 | 0.59 | 0.81 | 0.85 | 3.22 | 0.0017 |
| 345655 | 159 | 0.32 | 0.39 | 0.75 | 0.29 | 3.26 | 0.0018 |
| 347176 | 163 | 0.71 | 0.17 | −3.29 | −0.07 | 3.28 | 0.0018 |
| 352760 | 162 | 0.22 | 0.67 | −1.56 | 1.25 | 3.27 | 0.0018 |
| 348968 | 163 | 0.89 | −1.04 | 1.25 | −2.06 | 3.73 | 0.0019 |
| 345554 | 163 | 0.17 | −0.09 | 7.29 | 0.33 | 3.16 | 0.0021 |
| 342512 | 156 | 0.80 | −0.68 | 1.17 | −1.17 | 3.05 | 0.0024 |
| 344894 | 162 | 0.78 | −0.66 | 1.58 | −1.25 | 3.31 | 0.0024 |
| 353039 | 147 | 0.47 | −0.18 | 3.04 | −0.14 | 2.89 | 0.0024 |
| 353800 | 163 | 0.12 | 1.17 | −0.41 | 1.54 | 3.21 | 0.0025 |
| 354182 | 163 | 0.80 | −0.30 | −1.23 | −0.08 | 3.03 | 0.0027 |
| 348929 | 162 | 0.88 | −0.58 | 1.84 | −1.40 | 3.14 | 0.0028 |
| 354184 | 161 | 0.52 | −0.33 | NaN | NaN | 3.25 | 0.0028 |
| 354414 | 163 | 0.42 | −0.19 | 2.69 | −0.10 | 3.09 | 0.0028 |
| 346388 | 154 | 0.36 | −0.10 | 5.24 | 0.05 | 2.83 | 0.0029 |
| 349564 | 158 | 0.37 | −0.48 | −0.53 | −0.54 | 3.12 | 0.0032 |
| 351131 | 163 | 0.98 | NaN | NaN | NaN | 3.17 | 0.0034 |
| 347617 | 162 | 0.27 | 0.43 | −1.43 | 0.71 | 3.04 | 0.0035 |
| 347981 | 162 | 0.63 | 0.38 | 1.06 | 0.48 | 2.93 | 0.0035 |
| 344620 | 154 | 0.88 | −0.06 | 12.61 | −0.61 | 2.24 | 0.0036 |
| 344645 | 162 | 0.14 | 0.09 | 7.22 | −0.37 | 2.83 | 0.0036 |
| 343505 | 159 | 0.13 | 0.48 | −1.71 | 1.09 | 2.97 | 0.0038 |
| 348415 | 162 | 0.26 | 0.66 | −0.75 | 0.91 | 2.91 | 0.0038 |
| 343071 | 149 | 0.09 | 0.69 | −0.72 | 1.09 | 3.14 | 0.0039 |
| 343076 | 150 | 0.54 | 0.28 | 1.69 | 0.31 | 2.79 | 0.0039 |
| 353926 | 161 | 0.94 | −0.07 | 18.83 | −1.18 | 3.18 | 0.0039 |
| 348572 | 162 | 0.51 | −0.31 | NaN | NaN | 3.02 | 0.0040 |
| 353896 | 163 | 0.14 | 0.68 | −0.64 | 1.00 | 2.88 | 0.0040 |
| 348185 | 163 | 0.78 | −0.60 | 0.95 | −0.92 | 3.04 | 0.0043 |
| 352138 | 163 | 0.83 | −0.47 | 2.07 | −1.13 | 3.11 | 0.0043 |
| 345838 | 163 | 0.79 | −0.03 | −22.89 | 0.32 | 2.82 | 0.0044 |
| 346372 | 163 | 0.69 | −0.46 | 1.09 | −0.64 | 3.03 | 0.0044 |
| 347168 | 153 | 0.50 | −0.08 | −8.61 | −0.08 | 2.79 | 0.0044 |
| 342743 | 154 | 0.69 | 0.49 | 1.24 | 0.72 | 3.18 | 0.0047 |
| 354282 | 162 | 0.56 | 0.30 | 1.22 | 0.35 | 2.59 | 0.0047 |
| 342778 | 137 | 0.86 | −0.68 | −0.12 | −0.62 | 3.07 | 0.0048 |
| 342709 | 156 | 0.81 | −0.06 | −10.09 | 0.32 | 2.77 | 0.0049 |
| 353681 | 163 | 0.79 | −0.16 | 4.76 | −0.62 | 2.70 | 0.0050 |
| 348459 | 163 | 0.51 | −0.12 | 4.42 | −0.14 | 2.89 | 0.0051 |
| 351663 | 161 | 0.52 | −0.09 | 5.94 | −0.12 | 2.83 | 0.0051 |
| 353643 | 163 | 0.57 | −0.23 | 1.88 | −0.29 | 2.60 | 0.0051 |
| 351089 | 162 | 0.42 | −0.36 | −1.08 | −0.43 | 2.73 | 0.0052 |
| 344880 | 162 | 0.39 | −0.12 | 3.81 | −0.02 | 2.64 | 0.0055 |
| 350721 | 163 | 0.71 | 0.43 | 0.09 | 0.45 | 2.61 | 0.0055 |
| 353868 | 163 | 0.11 | 0.82 | −0.67 | 1.25 | 2.76 | 0.0055 |
| 346220 | 152 | 0.04 | −0.06 | 19.48 | 1.05 | 2.72 | 0.0057 |
| 354055 | 145 | 0.89 | −0.25 | 2.94 | −0.81 | 2.75 | 0.0057 |
| 342518 | 159 | 0.81 | −0.45 | 1.92 | −0.97 | 2.86 | 0.0058 |
| 342896 | 129 | 0.60 | −0.13 | −5.18 | 0.01 | 2.82 | 0.0058 |
| 344366 | 162 | 0.20 | 0.17 | 2.68 | −0.11 | 2.74 | 0.0058 |
| 348325 | 163 | 0.74 | 0.06 | 12.79 | 0.41 | 2.83 | 0.0058 |
| 351334 | 158 | 0.57 | 0.29 | 1.28 | 0.35 | 2.84 | 0.0058 |
| 352556 | 134 | 0.19 | 0.24 | 8.86 | −1.09 | 3.20 | 0.0058 |
| 352985 | 163 | 0.58 | −0.24 | 1.76 | −0.31 | 2.57 | 0.0060 |
| 350226 | 163 | 0.32 | 0.49 | −0.97 | 0.66 | 2.77 | 0.0061 |
| 349583 | 161 | 0.09 | 0.44 | −2.25 | 1.26 | 2.84 | 0.0063 |
| 348246 | 162 | 0.95 | NaN | NaN | NaN | 2.77 | 0.0064 |
| 349492 | 160 | 0.58 | −0.28 | 1.74 | −0.36 | 2.73 | 0.0064 |
| 345395 | 163 | 0.55 | −0.31 | 1.33 | −0.35 | 2.70 | 0.0065 |
| 349106 | 162 | 0.56 | 0.38 | 0.39 | 0.40 | 2.69 | 0.0065 |
| 349116 | 160 | 0.64 | 0.28 | 1.55 | 0.40 | 2.69 | 0.0065 |
| 354174 | 160 | 0.19 | −0.16 | 3.02 | 0.14 | 2.82 | 0.0065 |
| 342524 | 153 | 0.72 | −0.42 | 1.23 | −0.65 | 2.85 | 0.0066 |
| 349299 | 163 | 0.36 | −0.13 | −4.44 | −0.29 | 2.56 | 0.0066 |
| 353752 | 163 | 0.55 | −0.31 | 1.33 | −0.35 | 2.70 | 0.0066 |
| 343971 | 163 | 0.74 | −0.58 | 0.99 | −0.86 | 2.95 | 0.0067 |
| 346305 | 159 | 0.25 | −0.21 | 1.94 | −0.00 | 2.72 | 0.0068 |
| 353054 | 162 | 0.10 | 1.03 | −1.41 | 2.19 | 3.15 | 0.0068 |
| 353060 | 163 | 0.45 | 0.31 | −1.33 | 0.35 | 2.70 | 0.0068 |
| 343405 | 138 | 0.44 | 0.08 | 6.65 | 0.02 | 2.55 | 0.0069 |
| 346429 | 163 | 0.64 | 0.28 | 1.48 | 0.40 | 2.71 | 0.0070 |
| 348275 | 163 | 0.54 | −0.03 | 24.43 | −0.08 | 2.76 | 0.0070 |
| 345839 | 161 | 0.80 | 0.22 | −2.00 | −0.04 | 2.84 | 0.0071 |
| 351743 | 163 | 0.63 | −0.36 | 1.37 | −0.48 | 2.68 | 0.0071 |
| 345160 | 159 | 0.66 | −0.40 | 0.32 | −0.44 | 2.90 | 0.0072 |
| 346062 | 162 | 0.68 | −0.17 | 3.34 | −0.37 | 2.64 | 0.0074 |
| 352701 | 162 | 0.45 | 0.13 | −4.00 | 0.18 | 2.72 | 0.0074 |
| 350780 | 163 | 0.32 | −0.17 | 2.38 | −0.02 | 2.66 | 0.0075 |
| 351863 | 161 | 0.05 | 1.11 | −0.60 | 1.72 | 2.94 | 0.0075 |
| 350519 | 162 | 0.65 | 0.27 | −1.04 | 0.18 | 2.55 | 0.0077 |
| 345464 | 162 | 0.95 | 0.24 | 5.78 | 1.47 | 2.80 | 0.0080 |
| 351654 | 158 | 0.88 | −0.58 | 1.78 | −1.36 | 2.76 | 0.0081 |
| 354135 | 150 | 0.37 | 0.32 | 0.28 | 0.29 | 2.52 | 0.0081 |
| 347988 | 161 | 0.65 | −0.20 | −1.80 | −0.09 | 2.62 | 0.0083 |
| 348126 | 163 | 0.18 | 0.17 | 2.94 | −0.15 | 2.60 | 0.0083 |
| 352915 | 154 | 0.70 | −0.11 | 5.42 | −0.33 | 2.51 | 0.0083 |
| 349323 | 162 | 0.87 | 0.83 | 1.33 | 1.66 | 2.93 | 0.0084 |
| 351593 | 158 | 0.90 | 0.51 | 2.52 | 1.54 | 3.00 | 0.0084 |
| 347802 | 163 | 0.15 | 0.18 | 2.44 | −0.13 | 2.53 | 0.0085 |
| 350996 | 159 | 0.38 | −0.16 | −3.70 | −0.31 | 2.64 | 0.0086 |
| 349235 | 162 | 0.09 | 1.03 | −0.73 | 1.64 | 2.78 | 0.0087 |
| 351065 | 163 | 0.13 | 0.19 | −3.90 | 0.73 | 2.70 | 0.0088 |
| 343441 | 162 | 0.44 | −0.11 | 4.02 | −0.06 | 2.65 | 0.0089 |
| 343516 | 163 | 0.58 | 0.16 | 3.25 | 0.24 | 2.51 | 0.0089 |
| 342993 | 155 | 0.70 | −0.45 | 1.55 | −0.74 | 2.95 | 0.0092 |
| 349117 | 162 | 0.65 | 0.28 | 1.45 | 0.40 | 2.65 | 0.0092 |
| 346498 | 162 | 0.23 | 0.17 | −3.45 | 0.49 | 2.72 | 0.0093 |
| 346312 | 163 | 0.06 | NaN | NaN | NaN | 2.54 | 0.0095 |
| 346497 | 159 | 0.32 | −0.38 | −0.34 | −0.42 | 2.55 | 0.0095 |
| 343880 | 159 | 0.72 | −0.23 | 3.28 | −0.56 | 2.64 | 0.0096 |
| 345945 | 161 | 0.57 | −0.18 | −2.28 | −0.12 | 2.52 | 0.0096 |
| 345210 | 151 | 0.56 | −0.12 | −3.86 | −0.06 | 2.62 | 0.0097 |
| 354455 | 159 | 0.19 | 0.07 | −9.19 | 0.46 | 2.41 | 0.0099 |
| 350589 | 162 | 0.75 | −0.42 | 0.64 | −0.56 | 2.65 | 0.0100 |
| 351724 | 163 | 0.76 | 0.31 | 2.32 | 0.70 | 2.63 | 0.0100 |

TABLE 17

DNA sequences for each of the SNP ID in
Table 16.

| | | | |
|---|---|---|---|
| | | 5′flanking | |
| | | sequence | 3′ |
| | scaffold-v1/ | alternative | flanking |
| Snp_id | IBISS4 | bases | sequence |

SEQ ID NO: 352

| | | | |
|---|---|---|---|
| 342512 | SCAFFOLD | TATCTATCATGTCCCTGTCTTT | T/C |
| | 141737_19472 | TCTTTCTAAGGCATTTCCTGAG | |
| | | TCTCTGCACAGCTGAAATAGTT | |
| | | CCCTTATTAAGTAACCTTTTTC | |
| | | CACCTTCATTCCCCTTCAGGCT | |
| | | TCCCTGTGACCCCAGTTGAATA | |
| | | GTACCACATTGGGTTTCCTTGT | |
| | | GGGGGTGAAGACTTTGCGGTCC | |
| | | TTTCTCCAGATCAGCAGAAATA | |
| | | TGCATCTAGGAAACCAAACGAG | |
| | | CGTCTCTGAATTCCTCCTCCTG | |
| | | GGACTCT | |
| | | CACCCAACCTGAGCAGCAGGAG | |
| | | CTCCTCTTTGCGCTTTTCCTGG | |
| | | CTATGTACCTGGCCACCGTGGT | |
| | | GGGGAATGGGCTCATCGTTCTG | |
| | | GCCATCGGCTTGGACTCTTACC | |
| | | TTCACACCCCCATGTACCTCTT | |
| | | CCTCGCCAACCTAGCCTTTGCT | |
| | | GATATTTTTTCCATTTCCACCT | |
| | | CAGTCCCCAAAATGCTGATGAA | |
| | | TATTCAGACCAAGAGTCCATCC | |
| | | ATCTCCTATGAGAGCTGCGTCA | |
| | | CACAGATG | |

SEQ ID NO: 353

| | | | |
|---|---|---|---|
| 342518 | SCAFFOLD | CACAATACATGGTGGGAAGGCC | T/C |
| | 160139_23346 | AGGGGACTCAGAGTTCAGAGAC | |
| | | TGGGTTTCTGACCTTCTTTGCC | |
| | | ATTTTTTGCATTTGGTGCTTTG | |
| | | TGCTAAGTTGCTTCAGTCATGT | |
| | | CCGACTCTGTGCAACCCTATGA | |
| | | GCTATAGCCCGCCAGGCTCCTC | |
| | | TGTCCATGGGATTCTCCAGGCA | |
| | | AGAATACTGGAGTGAATTGCCA | |
| | | TTCCCTTCTCCAGGGGATCTTC | |
| | | CTAACCCAGGGATCGAGCCGGT | |
| | | GTGTCTTA | |
| | | GTCGAACCTGCATTGGCAGGCA | |
| | | GGTTCTTTACCACTAGCGCCAC | |
| | | GTGGGAAGCCCTTTTACGTTTG | |
| | | GGCTCTGGGTTAATTCACTCTT | |
| | | TGCCAATGTTCTTGCCTCTTGA | |
| | | TGCTTAATTCAAGCACCTTAAT | |
| | | TCAGCACTGACTTTATTCACAG | |
| | | AAAACAGTCTTCAGAACTGTGG | |
| | | ATTCTGTGCTGGGACTGTGCAG | |
| | | TGTTATCATTTCTTTTGAACTC | |
| | | CAGGTTATTTTGAAAACATGGG | |
| | | GTGTGTGT | |

SEQ ID NO: 354

| | | | |
|---|---|---|---|
| 342524 | SCAFFOLD | CCAGCAGTCCCACAGCTGTCAC | C/T |
| | 164825_5474 | CACGATCCTTTTAAAATCACAA | |
| | | TCTGATTCCATCACTCCCTAAC | |
| | | CTCAAACTTCCAACCACTTCCT | |
| | | ATCACATGTAGAATAAAATCCA | |
| | | AAGATCCTTGCCATAGTCTGGA | |
| | | GACCTTACATGTAGATCCCTGC | |
| | | TTATCAGAGACCTCCCTTCCTT | |
| | | CTGTGTTCACCATTACAGCCGC | |
| | | AGAGTTTTCCTGGATGGGCTCC | |
| | | AAACGTCCCAGGCCAGCATTCC | |
| | | TCCAGGGT | |
| | | GGGGCAGAGTGCTTTCTCCCGA | |
| | | CAGTCCTACACACACCAGAACC | |
| | | CTGCTATCACTCTCTATCCCCT | |
| | | CGCTCCGCTTTATTTCTCTACA | |
| | | CTGCACTCAGGATTATATTACA | |
| | | CACGTATTAGCTATTTCTCTGT | |
| | | ATACCACGTAACCACGAGGGCA | |
| | | GGGGTTGGATCTGTTTTGTTCA | |
| | | CAACTACATCAACAATGCTTAG | |
| | | GAGAGGTCTGGCACAAGATGGT | |
| | | CTCTCCAAAAGTATTTGGAGAG | |
| | | TGAATGAAG | |

SEQ ID NO: 355

| | | | |
|---|---|---|---|
| 342709 | IBISS4snp425 | ACTTTTTACGAGGACCGGGGCT | T/C |
| | | TCCAGGGCCACTGCTACGAGTG | |
| | | CAGCAGGACTGCCCCAACCTGC | |
| | | AGCCCTATTTCAGCCGCTGTAA | |
| | | CTCCATCCGCGT | |

SEQ ID NO: 356

| | | | |
|---|---|---|---|
| 342743 | IBISS4snp643 | CAGCCACTAGGTGGCGCTGCTG | C/T |
| | | AGCCAGGCCCACTCCCCTGGTC | |
| | | CACAGCGGGCCAGCAGTATCCG | |
| | | AAAGTGATTTCCATCCAGACCG | |
| | | ACATCCCTCTGA | |

SEQ ID NO: 357

| | | | |
|---|---|---|---|
| 342778 | IBISS4snp854 | ACTTTGCCCCCTTGTGCATCTT | C/T |
| | | CATAAACCTGCTGTGGGGACTC | |
| | | CTCAGCCGTAGGGGCTCAGGGT | |
| | | TTCCTGAGAGCCAGATGTCCTG | |
| | | CAGAGTTGCTGG | |

SEQ ID NO: 358

| | | | |
|---|---|---|---|
| 342896 | SCAFFOLD | AGGATCATTGCGTTTTTGGCAA | G/A |
| | 166534_2377 | AGAAGGAAACTGTGACTCAGAG | |
| | | GAGGGAAGCTCCTGGCCCAAGG | |
| | | CTGCCCTGAGCAGGAGAGTCGG | |
| | | GTTCGGGTCTGTGGGTCTGACT | |
| | | CTGTCCTTCCAAGTGCTCCAAG | |
| | | ATCTCCTCAGCTCCAGTGCACA | |
| | | CGCTGCAGATGCACAATGCTCA | |
| | | CATTCGCACACATACACATGCA | |
| | | TATGTATGCATGCACATGCACA | |
| | | CACGGCATGCACAAATGCATGC | |
| | | ACACACGC | |
| | | AACATGTATGCAGCCACGTGTA | |
| | | CATACATGGCCCACACAAATAC | |
| | | ATGCACACACATATGTATGCAC | |
| | | GCACATGTATACACATGATACA | |
| | | CACAAATACATGTGTATATATC | |
| | | CGCACACAGACACATATGCACA | |
| | | CATGTACATACATGAACACACA | |
| | | GGTACATAGGCACATGCACACA | |
| | | AACATACGTGCATGCATTCAAA | |
| | | CATACACGTGCACACACATGCA | |
| | | TAAACACGCATGTTTTCACATA | |
| | | CGTGCGTG | |

SEQ ID NO: 359

| | | | |
|---|---|---|---|
| 342993 | SCAFFOLD | AATATTTACAGTCTATGGAAAG | A/G |
| | 261578_4354 | AGGTAACTGACAAAAATGAGAT | |
| | | CTTTTATCACTGTGTCAAAGTA | |
| | | AATACACTGAGAAATATACTAA | |
| | | GATAACTTACTAAGATTACTAA | |
| | | GTGCAAAGCAGAAATCAGACAC | |
| | | AGATTCTAGGTCAAGTTTTTAG | |
| | | CTCAGAGGTTTATTTTGACCAA | |
| | | GGATTGCTTGCACGGTCTTTAA | |
| | | AGGTCACAGAGGTAAACCATGG | |
| | | GAGGGTCACTGGGATGAGACGG | |
| | | GCAATGCT | |
| | | CACAGGTTTCCCTGAGGCCTGA | |
| | | GTGCCATACAATGAAACCGAGT | |
| | | TGAGAACACAGCACTGTTCCGT | |
| | | CAAACTGCCTGATTCAGCAGGT | |
| | | GCTATGAACTGAGCATCAGTGC | |
| | | CCACACAGACAAGGTGGCTCCA | |
| | | GACACAGCAAAAAAGACAACAT | |
| | | GCTGGGAGCCAACGGCAAGAAA | |
| | | CGTCACCATCGTTCTCATTACA | |
| | | GCGATGGCCGCTTCTTCTTTAA | |
| | | TAACACGGTGTCCTTCTACAGA | |
| | | TAAAGTGT | |

SEQ ID NO: 360

| | | | |
|---|---|---|---|
| 343071 | SCAFFOLD | TATGAGATGCAGAATTCCAACG | G/A |
| | 41706_5862 | AATGCAAGAGGAACTTCGTGGC | |
| | | AGTGTATGACGGAAGCAGCTCG | |
| | | GTGGAGGACCTGAAAGCCAAGT | |
| | | TCTGCAGCACGGTGGCCAACGA | |
| | | CGTGATGCTCCGCACGGGGCTC | |
| | | GGGGTCATCCGCATGTGGGCTG | |
| | | ACGAGGGCAGCCGCAACAGCCG | |
| | | CTTCCAGATGCTCTTCACGTCC | |
| | | TTCCAAGAACGTAAGTGTCTCC | |
| | | GCTCTTCGCTGCTCGGCCTTCC | |
| | | AGTGGGTC | |
| | | GAGGGTGCAGATCTCATGCACT | |
| | | GTGAATGGTGTTTCCTTCATGC | |
| | | AGCTTGAGCCTTAATAAGTTGT | |
| | | TCAAACCGCTTAGATTCCCTTT | |
| | | CCTTCTCTTTGATCCAGAAACA | |
| | | GGAGAGTTACATATGGCCTTTT | |
| | | GATTACTTACATCAGCATATGC | |
| | | AGGGCTTCCCTGGTGACTCAGA | |
| | | TGGTAAAGACTCTGCTTGCAAT | |
| | | GCAGGAAACCCGGGTTGGATCC | |
| | | CTGGGTTAGGTAGATTCCCTGG | |
| | | AGTAGGAA | |

SEQ ID NO: 361

| | | | |
|---|---|---|---|
| 343076 | SCAFFOLD | TTATGTCACACCTTTGCAGGTA | A/G |
| | 55505_8978 | AAACAACAAAGCCCATTGATCT | |
| | | TATGAACCCTTTCATAAAATTT | |
| | | GTTCCAAAATAAATAAATCTAT | |
| | | ATTTTGCACCCAGAAGCTTAGT | |
| | | TTGGACAGTCTTTTTACTTGAA | |
| | | TTAATATATAATAACTCCTCTT | |
| | | GATTTGATATAAACAAAACATG | |
| | | AACCCAGGCACATCACCCTCTG | |
| | | CTTTAGACAACAGAGTGAATGA | |
| | | TCAGGCCCTCATCCCCCTCTCT | |
| | | CTGGATAC | |
| | | TACAGTCCAGCCGAGGAGATAA | |
| | | ACACACAGCTATGACTGTAAAG | |
| | | CGGACCTGACCAGAGCCACAGT | |
| | | CACACCAGCCGTGGCAGTTCAG | |
| | | AGTGCCCACGGGGGGAGGCTGT | |
| | | GGACCTCTCACCTGGACGGCAG | |
| | | TGACAAGTCTTGAGATTCACTC | |
| | | CAGACACATCTCAGTTTCTCCT | |
| | | TCATCCACTTCTCCACATGCAT | |
| | | TCTCTCTTCTTCGCTTTCTCAG | |
| | | GAGGTAGGGGTGAGCGCTTTCT | |
| | | ATCTCATA | |

SEQ ID NO: 362

| | | | |
|---|---|---|---|
| 343405 | SCAFFOLD | CCCTCCCTCCCTCCGGCTAATT | G/A |
| | 200102_19625 | CTCTGGAGCGTGGAAACCCACC | |
| | | AAGCTCACTCTCCTGCCTGGGC | |
| | | TTTCAAGACCTCCTCGAGAGGG | |
| | | GGGCCCTGTGGCCTCGTGAGTG | |
| | | GTGCAAGCCCTGTTCTAGGGTT | |
| | | TTACTGGTTCTCTGCATAAACC | |
| | | AGGGAATATTAGCTTCTTTCAA | |
| | | TCTTTCACTTTCTTATCATCGA | |
| | | CTCTGGACCACCAGGTTCCGGT | |
| | | CCGTTAAAGGACCACAACAGCA | |
| | | GAGTACAC | |
| | | AGAAATGCTGGGCTGGGTGAAG | |
| | | CACAAGCTGGAATCAAGATTGC | |
| | | CAGGAGAATCTGCTGTTATGCA | |
| | | GATAACACCACCCTTTTTGCAG | |
| | | AAAGCGAAGAAGAATTAAGGAG | |
| | | CCTCTTGATGAAAGTGAAAGAG | |
| | | GAGAGTGAAAAAGTTGGCTTAA | |
| | | AGCACAACATTCAGAAAACTAA | |
| | | GATCATGGCATCTGGTCCCATC | |
| | | ACTTCATGGCAAATAGATGGGG | |
| | | AAATGGTGGAAACAGTGACAGG | |
| | | CTTTATTG | |

SEQ ID NO: 363

| | | | |
|---|---|---|---|
| 343441 | SCAFFOLD | CCCCGAGCTCTGGTCATGTAAC | G/A |
| | 220465_3232 | CAGGCGAGAAGTTAAGGAAACA | |
| | | CAAGGGAGAAAAGTGGCAATCT | |
| | | CTGAGCTCCAAAGACATTGAAG | |
| | | GAACTCAACTCCACCACCCTGA | |
| | | AGGAGACACACTGGAATAAAAG | |
| | | TGTCAAACAGGGCCTCAAAGCT | |
| | | CTAGCTCCGCGTCCAACTCTAA | |
| | | GAGCAATCGATGCTGAAACAGT | |
| | | CCCCCAAAGGCAGCACGGAAGG | |
| | | GGCAGAACTGAGAGTGGCCTTG | |
| | | AAAGGCCC | |
| | | GGAAGGTTGAAATGGGCTGTGA | |
| | | TGAAGAAAGTCAACTTCCTGGA | |
| | | GGAAGTTACTAAGAGCAGGAGT | |
| | | CAGGCTCAGGGGCCAAAGCAGA | |
| | | AATGAAGAACACTGGGATTAGC | |
| | | AGCAAGTGAAAGAAGGCTTCTA | |
| | | ACCCCAGGATGAACAGTCATTG | |
| | | CCAGATCACACAGAGTTATCTT | |
| | | TATTTTCTTAGAAGTGACAGAA | |
| | | AGAAATCTGAGGTTTAAACAGA | |
| | | TGCCTGATAGTGGTGCTCATGA | |
| | | CAGACTGA | |

SEQ ID NO: 364

| | | | |
|---|---|---|---|
| 343505 | SCAFFOLD | GAACAGAGGGCCTGTTTCCAGG | G/A |
| | 257759_2817 | GGAGGATTACTGGGGAGACGTT | |
| | | CCAAGAAGGCGGGGAAGCGAGG | |
| | | CCCTGCCACGAAGCGGTCATCT | |
| | | CAAAGCCAAAGCTGGACAGATG | |
| | | GAGTCCCCACTGGGATCTGAGG | |
| | | ACCAAGCAGGGTCCAGGCAGGA | |
| | | AGCTCGGGAACAAATGCACAGC | |
| | | TCAGAGAGGGGACGGTGATCGG | |
| | | AGGCGTGTGGAGTGACCACAGG | |
| | | GCAGCACAAGCACTCAGATGTC | |
| | | TGTCCACC | |
| | | CCATGACCGGCAGAAGACGCAG | |
| | | CTTCCACAGTGCACATCCAAGC | |
| | | CATGTGACACGAGGCTGGTGAA | |
| | | GGTCGTCTCTCCTCGGCCCCAG | |
| | | GTGGAGAGAGGAGAGAGACAGC | |
| | | ACGTGCTTGGGAAGTCCAAGAG | |
| | | ACCTGAACTTAATTCCTGGCTC | |
| | | CCACAGCATCAGGATGACCTTG | |
| | | ACCCCGTTATCAAGACCACTGC | |
| | | TCCTGAGTTCCCTCATGCGAGA | |
| | | AATGTCATTAACGCCCTCCCAT | |
| | | GGATGCAC | |

SEQ ID NO: 365

| | | | |
|---|---|---|---|
| 343516 | SCAFFOLD | GATTCTATAACAGAGAATGAGT | A/G |
| | 265171_9373 | CTCTTCACATCAGGCCCCATAA | |
| | | GCATCTGTTACCTGATGTTCTC | |
| | | CTACATTTAATCTTATGAAACT | |
| | | ATTCTTCCCAGAATGAGTAATA | |
| | | TTGTTCCCTTCAACTCCATGCC | |
| | | TTTGCAAAATTCTCTTATTTTT | |
| | | AAAGAATGCTCTTCCCTGTTTG | |
| | | ATCTGCTTAGCCAATACCTACT | |
| | | CATTTTTTAAGACCCTGTGTTA | |
| | | TGCTTTCTTTGATCACTCCCAC | |
| | | CTTCCCCA | |
| | | GCTTCCTTCCCATGAGTTGACT | |
| | | ATGTTCTTGTGTCACTATCTCT | |
| | | AACACATATCCTTATCAAATAC | |
| | | TGTAATACGATTATGTACTGAC | |
| | | ATCTTGTCTCCCCCACTGTGAC | |
| | | ACCGAGAAGGGCCTGGTGGGAC | |
| | | TCCTGGGCACAAAGCCTTTCTG | |
| | | TGCCCATGACCTTCCCTGAGTT | |
| | | CCAAGGGGGAAGATACAACCAG | |
| | | TTGTTAGTTAGAGAAAGGANNN | |
| | | NNNNNNNNNNNNNNNNNNNNNN | |
| | | NNNNNNNN | |

SEQ ID NO: 366

| | | | |
|---|---|---|---|
| 343880 | SCAFFOLD | TATCATTTTTATGTCTGAAGTT | C/T |
| | 125746_1970 | TTTGAGAATGTACATGTTTACT | |
| | | TATAATATTGGGATATAAATTA | |
| | | AACTATTCTTTTAAAGCACAAT | |
| | | TTTATTAGAATATTTTATACCT | |
| | | ATACTATATGTTCTGAAAGTAG | |
| | | ATACTCTTTCATAGGCAATTGA | |
| | | AATATATATATAAAAGAAGAGA | |
| | | AAAAGGCAGGGGTGAGGTGGTT | |
| | | TGGCGTTTTGGGCTTAGCTTGT | |
| | | TTTTCACATTCCAGTGGTTTGG | |
| | | CTGTTTA | |
| | | GGCTCTTTCTCCAGTGCATTAG | |
| | | TTCCCCTGTGCTCCCATAACAC | |
| | | ACTGACACAAACTAGGTGGTTT | |
| | | AAGAACAAAAATTTATTGTCTC | |
| | | ACAGTTCTAGATGCTAAGTTCA | |
| | | AGATCAAAGTGACAGCAGGGCA | |
| | | TGTTCCTTCTAAAGGCACTGGA | |
| | | AAAGGATGTGTTCCAGCCCTCT | |
| | | TTCCTAGCTTCTGGTAGTGCCC | |
| | | AGGATTGTAACAGCATAACTCT | |
| | | AATCTTCACAGGGTATTCTATC | |
| | | AGTGTACA | |

SEQ ID NO: 367

| | | | |
|---|---|---|---|
| 343916 | SCAFFOLD | GGGGAAAATGCTTCTATTTTCT | A/G |
| | 140297_5422 | TTTAAATCCTGAGTTTAATCCT | |
| | | GAGTCATTATTATTTATCTACG | |
| | | GGGTCCAATGGACCCTTTTATT | |
| | | AAAAAGGGTCATTTTTAGGCCA | |
| | | TTTAACAAATAAATCAGAGATA | |
| | | AGAATAAGGGGAATAGTGAAAA | |
| | | TAGCTATATTAATTTTTTAGTT | |
| | | TATTATTATTTTTTTGGCCACA | |
| | | CCATGTGGCTTGTGGAATGTTA | |
| | | GTTCCCTGACCAGGGATTGAAC | |
| | | CCAGGTCC | |
| | | TCGCAATGAAAGCACCAAGTCC | |
| | | TAACCACTTGACTGCCAGGGAG | |
| | | TTTTCTATAGTAAAAATTTTAA | |
| | | TAATAATTTAAATAGTATTTGC | |
| | | ATTGTCAAAGGTAATATTTTGA | |
| | | AAACTTGGACTTTTATTGAATA | |
| | | GTGAACTGCTATCTTCTTTCCT | |
| | | CCATATTTACAAAACATTTAAA | |
| | | ATTTTAGATTTATGAGCACCAA | |
| | | TTAGAATGACTGTTTCAGTGAC | |
| | | TATTTTGGCTTATATATTATGT | |
| | | ATTTTCTT | |

SEQ ID NO: 368

| | | | |
|---|---|---|---|
| 343971 | SCAFFOLD | AAATTTCTTCCCTATTGACCCA | C/T |
| | 165459_3623 | GCGAGTAAAGAATCTGCCTGCA | |
| | | ATGCAGGAGATACAGGCGATGC | |
| | | GGGTTTGATTCCTAAGTCAGGA | |
| | | AGATTCCCTGGCGGAAAAAATG | |
| | | GCAACCCACTCCAATATTCTGG | |
| | | CCTGGAGAATTCCATGGACAGA | |
| | | GGAGCCTGAAAGGCTACAGTCC | |
| | | AAAGAATTGCATAGAGTTGGAC | |
| | | ATGACTGAGCGACTGAGCACAC | |
| | | ACGTTTCTAGGTAATGCTGATG | |
| | | CCTCTGGA | |
| | | GGAGGAACACACTTTGAGAACC | |
| | | ACTCTGCTGGAGCATTTAATTC | |
| | | AGGAGCCAAAATCTTAGATGGA | |
| | | CAAGAGGCCAGCGGGGGAGGCT | |
| | | GGGAAGTGGCGCTGGTGGGGTC | |
| | | CATGGCCAGCCAAGAAGCATGT | |
| | | ACCCCCTGAAAAGAAAACAGCT | |
| | | GCGCCTCAGCCACAGCCTGTGG | |
| | | TTGCCATTTGGAAATGTGCTGT | |
| | | GAGCGTTATTAGATTCTCCACT | |
| | | TGGTCTGGATAAAGTCTGGGAA | |
| | | TCTCAAAA | |

SEQ ID NO: 369

| | | | |
|---|---|---|---|
| 344366 | SCAFFOLD | TAAAAACAAAAGTATGTAGTAT | C/G |
| | 5882_5428 | GTTACTGATGACAGTGTTAAAG | |
| | | ATAGAAATAAAGTGGGGATAAA | |
| | | AAGATAAAACAGAGTACAATTT | |
| | | TTCACTGTTTGCAAAGGGAAGG | |
| | | TCTTTGGGAGGTGAAACAGCTA | |
| | | GAAATAAATGAAGGAGCCAAAC | |
| | | TTGAGCAGATGTGTGGGGGAGA | |
| | | AGGGGTTGGAGGAGCTGCTACA | |
| | | TGTACAGGGGGAGCAGGTGATG | |
| | | GAGAAGCAGCATGTGTACAGGG | |
| | | GGAGGAAG | |
| | | TGCAAATGCAGAGGGAATCACA | |
| | | GTATGAAGGCATCAGCATGGCA | |
| | | GTCTGGAAGTTCTGAGGAAAAT | |
| | | CACAAAGTCCATGAGCAGCGAG | |
| | | AGTGGAAGGAGGGCCAGGAGGA | |
| | | CAGACAGCAGGAGACGTGGGTG | |
| | | AGAATGGAGCAGGCAGAGGTGA | |
| | | AACGCTCAGTTAGAAATGGCCT | |
| | | ATGGCCACTCTGAGGACTTCTG | |
| | | ATTGTGAACTGTGCTCTGACTT | |
| | | CTGTTTTCACTCTGATTGCTCT | |
| | | TCTGGAAA | |

SEQ ID NO: 370

| | | | |
|---|---|---|---|
| 344620 | SCAFFOLD | CACTGTGCTGCTGCTGCTGCTA | C/A |
| | 130805_8260 | AGTTGCTTCAGTCGTGTCCAAC | |
| | | TCTGTGTGACCCCATAGACGGC | |
| | | AGCCCACCAGGCTCCCCCGTCC | |
| | | CTGGGATTCTCCAGGCAAGAAC | |
| | | ACTGGAGTGGGTTGCCATTTCC | |
| | | TTCCCCAATGCATGAAAGTGAA | |
| | | AAGTGAAAGTGAAGTCGCTCAG | |
| | | TCGTGTCCGACTCTTGGCGACC | |
| | | CCATGGACTACAGCCTACCAGG | |
| | | CTCCTCCATCCATGGGAGTTTG | |
| | | CAGGCAAG | |
| | | GTACTGGAGTGTAGTTCCTCTT | |
| | | TGTTCGTGCTTCCTCCCTTCTC | |
| | | CCCTCCTTCCTTCTTGGGTCAT | |
| | | AGCCAAGAAGTGCCATGCCTTT | |
| | | TCCAAGTCAAGTAGTGAGAATT | |
| | | CTGTTTGTCTTGTACAAACTCA | |
| | | AGGTAAAGTGTACCCTCACTTC | |
| | | AAAGGAAGCTGGAAATTCACAC | |
| | | TCACTGGAAGATTAGGCTTTTA | |
| | | GATGTTTTGTTTGTTCTTTTTT | |
| | | TCTATTTTACTTTTCCTCCCTT | |
| | | TTACAACA | |

SEQ ID NO: 371

| | | | |
|---|---|---|---|
| 344645 | SCAFFOLD | AACATGAAGATGGACATGGTTT | G/A |
| | 146059_2695 | CTTTGACAGGGCCTTGCAATTT | |
| | | GGTGGCATGCGTTCAGGGAGTG | |
| | | CCAACTCATTTGAATATCTGCC | |
| | | TAATTAGGAAGGTAAATTAGCT | |
| | | GCTCCTGCTGCCAGCAATCACA | |
| | | CCACGCACTGCAAAGCCAAGTA | |
| | | TACGTGCCTCTGGTAACACACA | |
| | | CGGTCATGAATTCTTCACCTCC | |
| | | CATGCCTATCCCTCCAGCAGCG | |
| | | CGGTGGACAATGCAGAAATGAC | |
| | | CAAACTGC | |
| | | TTCTCTGTCCGTCCTTTCCTCT | |
| | | CTCACCGAGGTTTGGCTCTATC | |
| | | ACGTGGACTGGAGTACAGATGG | |
| | | TTTAGAATTCTGAGGGCTTGAG | |
| | | GGGTGAGGTGACACAAAGGGAA | |
| | | TAAAGGACTTTGTTAACCCCGT | |
| | | CGACTTAAAAGAACGCACAACC | |
| | | TGAGAGTTGTGAGTTACGTTTT | |
| | | ATTTGGGGCAAAATGAGGACTG | |
| | | CAGCCCGGGAGGCAGCACCTCA | |
| | | GATAGCTCTGAGAAACTGCTCC | |
| | | AAAGAGGC | |

SEQ ID NO: 372

| | | | |
|---|---|---|---|
| 344880 | SCAFFOLD | CCCTAAAAGGTAGATTTTGACC | T/C |
| | 240874_16363 | AGTATATTTATAATGAATGTTC | |
| | | TAGGGAATGGGGGCTTTTGTTA | |
| | | AGAAACCAAAAAGAAAACCCAT | |
| | | TTTCCCATTGCATCCCTTGTTA | |
| | | TCCTAGTGGAAAGGGAAAGAAA | |
| | | GAGAACCCTTTTTACCCTTCCT | |
| | | TTTTACAGCTGGTAACTGAGGC | |
| | | ATAAAGTGATAAGTTACCCTCT | |
| | | GGGTGGATCTGGGACTTGAACC | |
| | | TGGCTCTTTGTTTCACTAAAGG | |
| | | GCAGAGGT | |
| | | CTTTGAGGGTGGAGCAATCACA | |
| | | TGTGATTCCTGATCCATCCTTC | |
| | | TGGGAGGGACATAGCAGAATAC | |
| | | TGCACTGTTAAAGGTGCTCAGC | |
| | | AACATCTCTATCAATCCAGTGT | |
| | | GCATCAGAATCCTTAGAGCACA | |
| | | CAAAGGGGGAACAAAAGGGGCA | |
| | | ATCTTCAGAAATATTCATTAGA | |
| | | CTTTCATGTTTAGAAAGTGCAT | |
| | | GTTTGTATGATAAACTGCTTAG | |
| | | CGAAAGTCCTAATAAAATCTTG | |
| | | ACTAATGA | |

SEQ ID NO: 373

| | | | |
|---|---|---|---|
| 344894 | SCAFFOLD | TCAACAAAGTAATGTCTCTGCT | G/C |
| | 245260_13971 | TTTTAATAAAATAGCAGTTAGT | |
| | | TTCTGCTTCCAACTATACCCCC | |
| | | TCTGAATACTTAATAACACGGT | |
| | | GTAGCCCAGCAAGTGCCACAGA | |
| | | AGACTGGAGTTTGTGTCACTGT | |
| | | AACACTATGCAGTGTGGACCCT | |
| | | TGAAGAAGAATGGGACTGCCCA | |
| | | CATTGTCTGCACCCTATCTTTA | |
| | | GTGCAGATGGGATCATCATAGG | |
| | | CAAGTTGCTGTCCTTCATGGAA | |
| | | AACACCAGC | |
| | | AGGGAAGTCTGCGATACTTTCA | |
| | | GAGCCTCCATCTCAGGGCCTCG | |
| | | GATAGTAGCATCAGATGTTTGT | |
| | | AGCAGGAAGTCAAATTTAATCG | |
| | | GTTTCTCTGTGACAGATTTAAA | |
| | | GTCCATCAGGTCCTGGGGAAAG | |
| | | ATCATCTGAAGAGCCAGAAGAT | |
| | | GAGCTCATTTGCCTCTCCTCAA | |
| | | AGACAAGTTAGTGAAAAAATTC | |
| | | AACAGAGGAGTGTTTAGCAGGT | |
| | | GGAAGCTCATAAATCTGTCAGC | |
| | | AAACTTTA | |

SEQ ID NO: 374

| | | | |
|---|---|---|---|
| 345109 | SCAFFOLD | TAGTCAGGAGACTGAAGCTATA | A/G |
| | 381707_559 | TGAGGGGTACTCCCTTCCCCAA | |
| | | GGCTGAGATTTAGAACATATTG | |
| | | GAGAGACTATGGTTTGGCTGCT | |
| | | GGTGGCAGAGAAGTTTACTGGA | |
| | | TGCCTTGGGCTGCAGTTGGTGT | |
| | | CCATTGGGATGCTGAGAAAGTT | |
| | | TGCTGAAGAGCAGGTGATAGTG | |
| | | GGCTTTCTGCATGCTCCTGGAA | |
| | | GCTTTGCCATCAATTGAAGACA | |
| | | ATACAAACTGGGAAGAAGAGAA | |
| | | GCCCCTTT | |
| | | TTTGTTACCTTGCCGTGTCTCT | |
| | | CTAAGTCATGTCTGACTCTTTG | |
| | | TGACCCCATGGACTGTAGTCTG | |
| | | CCAGGCTCCTCTGTCCATGGGA | |
| | | TTGCTCAGGCAAGAATACTGGA | |
| | | GTGGGTTGCCATTTCCTTCTCT | |
| | | AGCGGATCTTTCCAACTCAGGG | |
| | | ATTGAACCTGAGTCTCCTGCAT | |
| | | TGCAGGCAGATTCTTTACTGTC | |
| | | TGAGCCAACGGGAAGCCCAGTG | |
| | | TCGCTCTAGAGCCCTTTACTGA | |
| | | TGAAGCCT | |

SEQ ID NO: 375

| | | | |
|---|---|---|---|
| 345160 | SCAFFOLD | TTGGGCTTGATCAGAAGAGAGA | A/C |
| | 60025_24520 | GGGAGCAAGAAGTAAAACTCCT | |
| | | AAGAAGCTCTTGCATTTGGATA | |
| | | GAAATTCATCTCACCTGATGCA | |
| | | CATACACCAGCCTTGTGACTAG | |
| | | GCCAGGGAAGCTTGAGGAGGTG | |
| | | AGTGCCCAAAGGAGGGTGGTGG | |
| | | CAACAAAAACAAACCAGAGGAA | |
| | | GATGGCTTGAGTGCAGCTTGAC | |
| | | TTGGTGATTAGTTGCGGAAAAT | |
| | | AGAAAAGTCAGGTTGTGTGTGG | |
| | | GTGTCTGA | |
| | | AAAGGCACATGAGCATTCTGAG | |
| | | GACAGAACTTTTAAAAATAGCT | |
| | | CCCATCCCCTTTATACTATAGA | |
| | | ATAAATAGAACAAAGCATTTTG | |
| | | AACTGTTTTACAACGATTTCCC | |
| | | CATGGGTAGATAAGCCAGTTCA | |
| | | TAAAGATCTATATAAATGAACA | |
| | | CACGTATTTTTTATTTGTCACA | |
| | | TGCTCTATTTGTTTCATTTTTT | |
| | | TCCCAAAGCCATCTGTGCCTGT | |
| | | CCTTGACTTTGTTCTCTTTTGA | |
| | | CTAGTCTT | |

SEQ ID NO: 376

| | | | |
|---|---|---|---|
| 345210 | SCAFFOLD | GCGCTCTCCAGTGGCAGCCAGG | C/T |
| | 76733_4424 | GCCACCGCAGTTTGCATCCAGG | |
| | | GGCTGGCCTGTGCCAAGCACCC | |
| | | ACCGGAAGCCACCAGCCGGCTG | |
| | | AGGGCCCCTCAAGGGACTCAGG | |
| | | TTGCACATTTGCTATTTTGATC | |
| | | CAACAATGCAGGCCCCTTCCGG | |
| | | TTTCAGGAGCCGGTCTCCTGGC | |
| | | ACTCATCAAGCGCCCAGCTTCG | |
| | | AGAGGCTTCTGAGAGCCAGGGA | |
| | | GCAAGCTTCCAGCCTGAAGGGA | |
| | | GGGTGATG | |
| | | GCTCCTGGTTTTAAATCCCCAA | |
| | | CTGAATGCCAACCATATCTGGT | |
| | | TGTCAGCAGAGAGGGGAAGATC | |
| | | AAGAGGCAGTGATGGGAGAGAA | |
| | | TGGAGAGGTGGTAACAAGAGCT | |
| | | AAAATTTATTGCCCTGAAGCTT | |
| | | GTGTTCTCCTGGGAGGAGAGGG | |
| | | CACTGCCCACTGGGCCGAGGGC | |
| | | TCTTCTGTACCCGGCACTGCCT | |
| | | TTGCCATGGGGTGGTGGGGGTG | |
| | | GTTGGGGGGGGTGGTGTTCCTT | |
| | | GAGTGGAG | |

SEQ ID NO: 377

| | | | |
|---|---|---|---|
| 345395 | SCAFFOLD | ACAATCAGAAATTTATTATCTT | A/C |
| | 125013_22246 | ACAGCAAAGTAATGATAATTCT | |
| | | GTTCTTTACAACATGCGATACT | |
| | | ATAAAATCTTTATCTTGGAATG | |
| | | TATTCTTTGAAATTAAATGCAT | |
| | | CAGAGCAATAATATAGTCTCCA | |
| | | TGATGTTAGGATCATCCTTAGC | |
| | | AAAATTCTAATGGGAAATGTAA | |
| | | ATACCAGATATTTCTGTTTCCC | |
| | | AGTCATCTCTAAGACTGTATAA | |
| | | CACAAGAAACTGGCTTATTTGA | |
| | | CATGGCTG | |
| | | TTAGACAGTCGTCTTAGGAAAT | |
| | | ACTTATTCATGGGGTGTTCTAC | |
| | | CTGTTTTCCTTAAACAAAACTA | |
| | | AATGTTGTAGCAGGCCCACCCC | |
| | | TCACATTACGATGGGTAGAGGC | |
| | | GAGTAAAAACGGAAGCTGTATG | |
| | | TTTAAGTATTTAAAAGCTAACA | |
| | | GAGTTTTAAATAAAGTATGTTG | |
| | | GCTTTTCCTACCTTGACAAATA | |
| | | CAACTTCATGACAGTTTGGAAT | |
| | | ACTGTACCTGAATGTGGGAGCA | |
| | | TAGGAAGC | |

SEQ ID NO: 378

| | | | |
|---|---|---|---|
| 345464 | SCAFFOLD | AGTTAAGTTTTCAGTCTTATGA | C/T |
| | 150213_25683 | AAGCAGCCTCAATGAGTGTTTC | |
| | | TGCCTGGCAGTCTCAGTTGCCA | |
| | | CACTGCTTTGCCAAATTATAGT | |
| | | TTATCTGCACTGTGTTTTCTGG | |
| | | TCACGGGCTTCAGCTGAGCAGG | |
| | | ACGAGACTCTGAGGACTGAGAC | |
| | | CCACATATTCAGACCATGGGTG | |
| | | CTCCCTGGGCTGCTGCCGACCC | |
| | | ACTGGGTCTGGAAGCACAGGGG | |
| | | TCACAGTGGGACTAGGCTAGTT | |
| | | CTTTTCCA | |
| | | GGTGGTTGCTATTTCCAGCTCA | |
| | | TGTTTATGAAATACATATAATT | |
| | | TTAAGGCTTTGCCAAGAGAGAG | |
| | | CCTTCCCTCTGTGGTTGATTTT | |
| | | CAGAGTCATAATTACCCTCTTA | |
| | | TTACAAGTCAGTGTTCTGGGCC | |
| | | CTGACTCCCCTGGGTTCTTGTG | |
| | | GCCGGCTCGTGGGACAGACAGA | |
| | | GGTATAACACTGGTCAAATCTG | |
| | | CTGACCACGCTGTTCCGAGCTA | |
| | | GCAGACTCGGCAGCCTTGTCAA | |
| | | GGGTTTGA | |

SEQ ID NO: 379

| | | | |
|---|---|---|---|
| 345554 | SCAFFOLD | ATGGGATTTTCCAGGCAAGAGT | T/C |
| | 175391_5819 | ACTGAAGTGGGTTTCTACCAAT | |
| | | GAACTATTCCACCCACCAAAAA | |
| | | TAAAAGACTTTCCATTGGGTCT | |
| | | CTAGAGGTAGGGCCCATTGCTG | |
| | | CATGGTTCATTGCTGTATTCCC | |
| | | ACCACAGTGCCTGGCATAACTA | |
| | | AGACATAATATAAGCATGTAAT | |
| | | GAAATAATATGGAGTTGATGCT | |
| | | GCTAATCATGGAAGGTGCTACA | |
| | | AAATGAGGCCTGTGTCCTCGAT | |
| | | TACAATGG | |
| | | TCCTCTTCCCTGAGATCCTCCA | |
| | | CTTCTCCCATCCCTTTCTGCAT | |
| | | CCCATTGTGGACCATAAGGAGG | |
| | | CATGACTAAGGCTCCACGTCTG | |
| | | TCACCATGGCTGCAGGCCCACT | |
| | | TGAAATGCTGAGGGCACTCTGC | |
| | | CTTCACCAAGGCTATCCAGAAC | |
| | | AGCACACAAACCCTACAGTTAA | |
| | | GAGGTCCAGGGACTTCCCTGGT | |
| | | GGTCCAGTGGCTAAGACTCCCT | |
| | | GCTCCCATTGCAGGGGTTCTTG | |
| | | CTTCGATT | |

SEQ ID NO: 380

| | | | |
|---|---|---|---|
| 345655 | SCAFFOLD | GAACGTGGGATGGGGGTTGCCT | T/G |
| | 213107_5745 | CCACTAACTTGTGATGCCAGCT | |
| | | TGAATGTGATTCCTAAGTTCTT | |
| | | GGAACATCTTGGTTTCCTCATC | |
| | | TGAAAAATACAGGCAGGGTTGT | |
| | | AGTAGTGAGGAACGAACCTGCC | |
| | | ACACAGTGCTAAGTAAATCCGA | |
| | | ATCTCATGTTAAAAAATAGAGT | |
| | | AGTTACTGGAACATATTTGTCT | |
| | | ACATAAAGGTCCTCTGCATGCA | |
| | | ACGTAAATCTCCTTAAGACTTA | |
| | | TTGCTGCT | |
| | | CTTCTACAGTTCCTGGCACATA | |
| | | ACTGCGACGCTGGTGAGAGAGA | |
| | | CAACAGCTACATCTTGTGCGAT | |
| | | GCCATTCAGATGACATTCCAGA | |
| | | GAAGGTAAAATTACAGGGATAG | |
| | | AAAACAACACATCAGTGTTTTT | |
| | | TGTTTTTTTTTTTAAGATGACT | |
| | | GGGAATTTGGTTTATGCCAAGT | |
| | | TACCTGAGATTACTATTTTTAA | |
| | | AAGATATTTATTTATTTTTGGC | |
| | | CACACTGTGTAGTATGTGGGAT | |
| | | TTTAGTTC | |

SEQ ID NO: 381

| | | | |
|---|---|---|---|
| 345838 | SCAFFOLD | GCCCAACATCCGCTGATGGAAG | A/G |
| | 271271_15396 | CCAAGCCGTGAGACCCATTATC | |
| | | TCCTCATCTTTGTCCACTAGCC | |
| | | TTCCAGAGTGACCTCTGCATCG | |
| | | CTCACTTCAGTCTCTGGGTTCT | |
| | | TTCACTTCCTCCCTGGGGATTG | |
| | | TTCTGCAGTCACTTACAGAGGT | |
| | | GCTCTCGAGACCTTGCTGTCCT | |
| | | TCAGGTCTCCACCTTAAACCAA | |
| | | GAAAGCCTATCTCCAGTCCTGG | |
| | | CAGCTTTCTTACTTTGCTCTTG | |
| | | TGAAGCTC | |
| | | TCTTTCAACCTCACAACTCCTA | |
| | | ATCCAGAACAACCTCTTATTCT | |
| | | CCTCCTTAGAGAGTTCCCCATG | |
| | | GCTTAATTTTTTTACCACTTGG | |
| | | ATCCCATTGGCTAGTGCATTCT | |
| | | CAGCTGTACATTCAAACCATAG | |
| | | TGAGGTATCATTACATACCCTG | |
| | | TAGAAGGCAGAAAAGTAAAAGG | |
| | | ACTGACAGTGCTAGGTGTTGAT | |
| | | GAGATATAAGACAAGGAGCAAC | |
| | | TAGAACTCTGGTGGGAATGTAA | |
| | | AGTGTTAC | |

SEQ ID NO: 382

| | | | |
|---|---|---|---|
| 345839 | SCAFFOLD | AGATCAGGAATGAGGCACTCTG | A/G |
| | 271317_5508 | TGCTCTGGAAAAATCTGGCAGA | |
| | | ATGGACCTTCAGAGAATTAGAT | |
| | | ATTTTCTGGAGAGGATTTTATG | |
| | | AACCCAATTGCTCGCATCTTCT | |
| | | CATATCTACAAAAGTACTAAAA | |
| | | ATCATTAATGGGGACAGCTCTC | |
| | | CCTCGTGACTAGGAGTGACCTT | |
| | | CTTGTGACTAGGAGAAAACCTC | |
| | | TACCAAAATGTGTGCTTGACTG | |
| | | CCCATATCCCCCTTCACCAAAA | |
| | | TCACGTCT | |
| | | TACTGACCTTCCCTCCAGAGCA | |
| | | GTTCCGCAGAGCTCTCTGAGAG | |
| | | GCTGTCTCCTGGGCTAGAGCCC | |
| | | ATGTTTTGCTCCAAATAAAATT | |
| | | TAACTCACAACCCCCACATTGT | |
| | | GTTTTTTTTTCTCAGTTGACAC | |
| | | TGTCGAGGTTGCAAAGTGAAGT | |
| | | CATGTCTCCCCTGGCTTCCTCC | |
| | | AGCCCAATCATGTTTTGTTTAC | |
| | | CTCACTCATCTGGGAAAAAAAA | |
| | | AAAAAATCAATTTTCCCAACTA | |
| | | GCAATCCA | |

SEQ ID NO: 383

| | | | |
|---|---|---|---|
| 345945 | SCAFFOLD | TAGGTTTGAGCCTTAAACTAAA | T/C |
| | 313403_3761 | CTAAACACTAGCAAAGACTGTG | |
| | | CCACATGGACAAGCACCACTGA | |
| | | AAGCAGATGCCAGTACTAAAGC | |
| | | GAAGAGCCCAGAGGAAGACCCA | |
| | | TCAGGTTCCAGGATTTTCTGCA | |
| | | CCCAGAATGTAGCAAGGCCTGG | |
| | | CTTGGATTCCATGAGATTCATA | |
| | | TAGCATATTTTACTCTTCTAGC | |
| | | TTCTTATTCTAGACTCAATTTT | |
| | | GTGTTCTCATGGTTGGGGTTCT | |
| | | GTTTTCTT | |
| | | CCCTTCTAGACAGCTCACCTGA | |
| | | AGATCTTATTTTCTGCAAAGAT | |
| | | GTGAATCTTTTCTGTGAACTGT | |
| | | TTAAGAGGCAAGCTTTTAGGAA | |
| | | TACAGGATCATTTAAGGAAAAT | |
| | | CTGGTTTTACAAATTTAAATTT | |
| | | ATTCAGGGCGAAAAACAAGCCA | |
| | | TTTCACAAGTCTGCGATTGATA | |
| | | AAATTATGAGAAAAATCTTAAG | |
| | | GAAGAAACACAGGCCTGAGGGA | |
| | | TTTGGATAATAATAACCTGCAG | |
| | | CTGCTTTT | |

SEQ ID NO: 384

| | | | |
|---|---|---|---|
| 346062 | SCAFFOLD | AGAGTCACTAATAAGGATCATT | A/G |
| | 56166_5495 | AAGAATTTATTGTAATATAGTA | |
| | | CCCCTTGTATGGAAAGCTAAGA | |
| | | GCACCAATTTAGGCGAAATAGT | |
| | | AAGATTTGGAAGTTAGAAGGGA | |
| | | CTTTAGATAGTCATTTCTCATA | |
| | | TTGGTATTTAGGGTTTAATTTT | |
| | | TCAGAGAGGTTTTGGGTACCGT | |
| | | ATTTCATTTACACCTTAGTGAT | |
| | | CCACTCCACGGTGGCATGTGTT | |
| | | TCTTATATTATCCCCATGCTTC | |
| | | TAGCAGAT | |
| | | TTTATAACCTGTCAGGACCCTC | |
| | | TTTCCTGCTAAAATTGTTGTTG | |
| | | TTCAGTCGGTCAGTCATGTCTG | |
| | | ACTCTCTTGTGACCCCATGGAC | |
| | | TGCAGCACGCCAGGCTTCCTTG | |
| | | TCCTTCATTATCTCAGGAAGCC | |
| | | TGCTCAAACTCGTGTCCATTGA | |
| | | GTCAGTGGTGCCATCCAACCAT | |
| | | CTATCTCACCCTCTGTCACCTT | |
| | | CTCCTCCTCCAGCCCTCTGTCT | |
| | | TTCCCAGCATCAGCGTCTTTTC | |
| | | CAGTATGT | |

SEQ ID NO: 385

| | | | |
|---|---|---|---|
| 346220 | SCAFFOLD | ATGTTGGATGGGCCCTTGTGGT | G/T |
| | 100297_8761 | TCTTATTACTATTATAGAATGC | |
| | | ATCTGAGCAGCAACCATCCGGG | |
| | | GGCTTTGAAACAATAAGGATTA | |
| | | TTAGTCTCATGTCCTGTGGCCT | |
| | | AACAGGACAATGGCCAGTGGGG | |
| | | AGGAAGGAGAGGGAGCACAGAC | |
| | | CTGGAGTTCTGCCTTTATTAGA | |
| | | GTCCAAGGGTGCTGTGGTAGGC | |
| | | AGGAACCAGGAGGCAGGAAGAG | |
| | | AAATGTAGTTATCTAGATTCCC | |
| | | TAAGGCTT | |
| | | CTAGAAAGGAGAACTTCGTGGC | |
| | | AGAGGGCGCAGAGAGGGCTGGT | |
| | | CTAAGAGCTTCCCTAGAAGCTG | |
| | | TGATGTATTTATGCAAGATGGG | |
| | | TGTTTGAAATGGATACTTTGGC | |
| | | AATCAGAAGTTTAACGCCAGGC | |
| | | CCTTAGGATCAGGCATTTTCCA | |
| | | TCATGCCTCAGCATTTAATATT | |
| | | CCCACCAAAGTGCAAAAGGCCT | |
| | | CCTTTCTCCACGTCCTCATCAA | |
| | | CACTTGTCAGTTTTCGTAGAGT | |
| | | GTGAGGTG | |

SEQ ID NO: 386

| | | | |
|---|---|---|---|
| 346305 | SCAFFOLD | GAGCCTGGTGGGTTACAGTCCA | C/A |
| | 131537_8388 | TGGGGTCACAAAAGAGTCGGAC | |
| | | ACGACTGAGCAACTTAACAACA | |
| | | ACTTAGCTCCACTGACTTTTTT | |
| | | CCTCTGGGAGTGAAACTAAGGG | |
| | | AAGGGTGGGGTGGGGGAAATTA | |
| | | TGCATGAAAACCTCTCAGTCTG | |
| | | AAGACTTATTACCTAAATATAA | |
| | | ATCTCACTACATGAAAGGAGAG | |
| | | TGATTGTTCTGAGGCATCAAAA | |
| | | ACTGTCATTTTTGTTTAGTGAG | |
| | | AGATAGTG | |
| | | GTCGTCGCGGTTAGGAAAGTAG | |
| | | ACTATTCTGCCAGGCTGCCAGG | |
| | | TTCAAACCCCTTCTCTGCCACT | |
| | | CACTATGCTTGTGATTTGGGGC | |
| | | AAGTTCTAAAGTTTCTGTGCCT | |
| | | CAGTTATCTAGTCTGAAAATGG | |
| | | GTGTGATAATTGGGTTGTTCCA | |
| | | AAGGTGAAATGCATTTAACAAG | |
| | | TTTAAATTGTTTAGAACAGTGT | |
| | | CTCACTGATAGTAAGTGCCCAG | |
| | | TATATATTAGCGACTATTAGCC | |
| | | AACTTCTA | |

SEQ ID NO: 387

| | | | |
|---|---|---|---|
| 346312 | SCAFFOLD | TGACATTCTTTCAATTTTTCTA | G/A |
| | 136071_11139 | TATTTTTGAAAATTTTAAAAGT | |
| | | TAAATGTTGAAGGCCAGGGGAA | |
| | | GAGTGAATCTGAACAACAGATT | |
| | | GTTTACAGCAAATATCTGAATT | |
| | | TTCACTAATGGTCTATGCCTAC | |
| | | ATTGACTTTGATAAAATGATTT | |
| | | CAAATCATTTTATTTTGATAGT | |
| | | GATTTTCAAAACTTGCATGTGT | |
| | | GCAAAGATTGTGTGCACACTTT | |
| | | CCAGGAAGGAGACGCTCCCACA | |
| | | GGTAAAGA | |
| | | TGGAGGTAGTGAGTAATACTTA | |
| | | GATATTGTTGGTAGCCAGTGTG | |
| | | TTGGATACCTGAGATTTGGAGG | |
| | | ATATGGCCATGGGTGTTATTTT | |
| | | AACTTTTAACCCAATAACAATG | |
| | | TGCTTCACTTTTGCTTCAGCCA | |
| | | CAGTGGTGGCAACAACAGCCTA | |
| | | GAAGAGTAATCCAAGTCTTTGG | |
| | | GTTGTCATCTTCAATAAAGGTA | |
| | | TTCTTATTCCTCTATTGAGGGA | |
| | | ACTCTGCTCAATATTCTGTAAT | |
| | | AACCTAAA | |

SEQ ID NO: 388

| | | | |
|---|---|---|---|
| 346372 | SCAFFOLD | ACAAAAATACTTTTTAAAATAT | A/G |
| | 157428_2012 | CTTCAAAAATCTTAGAGATGAA | |
| | | TATGACAAGGTGTTTGCAAGAT | |
| | | TTTTATACTAAAAACTATAAAG | |
| | | CATTGCTTAAGTTTCCTAAAAG | |
| | | AAGACCCAAATAAAAGGACAGA | |
| | | TATGCCATGTCCACATGTCTAA | |
| | | AAACTTAGTATTGCTGCCATGT | |
| | | CAGACTCTCCCAGATTGATTGA | |
| | | TTGCTTGGCCGTACCCTTTAGG | |
| | | CTTAACACCCCGAGACAGCACT | |
| | | TCATCACT | |
| | | AGTCATGAGTTAGACTCCTCTA | |
| | | CCTGTTTTTTATTTTCAGTTCA | |
| | | GTTCAGTCTTTCAGTCATGTCT | |
| | | GACTCTGTGACCCCATGGACTG | |
| | | CAGCACGCCAGGCTTCCCTGTC | |
| | | CATCACCAACTCCCAGAGTTTA | |
| | | CTCAAACTCATGCCCATTGAGT | |
| | | CAGTGCTGCCATCCAACCATCT | |
| | | CATCCTCTGTCATCCCCTTCTC | |
| | | CTCCTGCCTTCAGGCTTTCCCA | |
| | | GCATCAGGGTCTTTCAAATAAA | |
| | | TCAGTTCT | |

SEQ ID NO: 389

| | | | |
|---|---|---|---|
| 346388 | SCAFFOLD | AAAATGAGAAAGATAAAACCAT | A/G |
| | 164168_2380 | TGATTCTAAAGCTTAGAATGGA | |
| | | GCCAGCTCAGGCCACTGTCAAA | |
| | | GGCCTGGTCACTGAGTGAGAGA | |
| | | GGGCAGAGAGGAAGCTGTTACA | |
| | | GTTTGGGGGCTGTGTGCAGATT | |
| | | CTCATGCATTAGGGCTGAGGTC | |
| | | CAGCAGGAAACCTGAAATTGGG | |
| | | TTCAGATTTGGACCTTTCTTTG | |
| | | TGGAGATGGCATAACTTAACAG | |
| | | CAACTCGGTTTTAGGGAATGAC | |
| | | TGAAGAGA | |
| | | GATTGAGAAAGGAGCAGAGAGG | |
| | | AATCTTGTGTGCCTTTCCTTAG | |
| | | AAGTCAGCAAATCTCTTCCTTA | |
| | | CTGGTTCATTATAGATATTCAA | |
| | | TGGCATAATGCAGGGAAAGTAC | |
| | | AGATAACAATGCTGAAAATAGA | |
| | | CCTAGCTACGATAAGTAGGAGA | |
| | | CGGCAGAAATATTTGTTGAATC | |
| | | GACTTTATCATTTTCTCAAATT | |
| | | GTCTATCCTGCTTTCTTTCCTT | |
| | | TGTCTTCACTGCAGGAAATTGA | |
| | | GTTTTGGG | |

SEQ ID NO: 390

| | | | |
|---|---|---|---|
| 346429 | SCAFFOLD | AGTTAAATAAATCTGTATCTGT | C/T |
| | 1747_489 | TTATTGAAACTCCTTAAGAGAA | |
| | | AAGGAATGATTAGCAAATTACT | |
| | | AAGAGGCGGCTTTTCTCTTTGC | |
| | | TAGATTTAACCAGGAATTTGTG | |
| | | TGTGTGTTTGTATGTGTGCACT | |
| | | GTGTTGTGCGTTTCATTAATAT | |
| | | TCCTCTTATTTTTATTATTTAT | |
| | | CCTTCTCATTCACTGTTATGCT | |
| | | GAACATAGCCAGAAAATGAGGT | |
| | | CAACTCAACATTTGCTTTTGCT | |
| | | CAAGGCTA | |
| | | ATCAGGCAAATGAATTCTTTGT | |
| | | CCAATTCAGAAGAGAAATAAAT | |
| | | CAATTACCCTTGCTGAAGGGTG | |
| | | GCTATCTTGAGTGCTTTTAGGA | |
| | | AAAACTCAGGTCATTCTAAAGA | |
| | | AATGTCTGAAATATCTGCAAGA | |
| | | GTTAAGAGCAAGATAAGCACTA | |
| | | ATTTACCATCTCCTTCATCCCC | |
| | | ATAAGGAGGTACTTAAAAGAGG | |
| | | ACAAAATGCATCACTAGGTATT | |
| | | GTCAGGGTCAAACTAAGACTTC | |
| | | AGTATTTC | |

SEQ ID NO: 391

| | | | |
|---|---|---|---|
| 346497 | SCAFFOLD | TCATTTGATTCTAACATTTGGA | A/C |
| | 196368_795 | TTATGGACATGATGTTGCTGTG | |
| | | CTGTCCACGGATTATATATCAG | |
| | | TGTCTAGGCATCTCACACTCAT | |
| | | GTCCACAATACAGCTGTGTAAC | |
| | | GAGGAGCACTCTGGACATTGGC | |
| | | ATCTGGCTCATAGCCAGGAGAG | |
| | | GCTCTGCCCACCCAGCCCCTCC | |
| | | AATGGGGGGACAGTGCCTTTTG | |
| | | CCTCTTGCATGGTGGCTGGGTG | |
| | | GTGCTCACATCATAAACCACAT | |
| | | CCTGGAGG | |
| | | CTTCCAAGTGTAACTGCTCATT | |
| | | AAAGCCTATTTCTGAGGGCCAC | |
| | | GGGGTTGGGTCACTGCAAACCA | |
| | | TCCACCAGGTATGAAGTGGAAA | |
| | | GCAATGGCCTTTCAGGAACACA | |
| | | CGTGGATCAGTGATGAAGGACT | |
| | | GGATTGGCCAAAAAGTTAGTTT | |
| | | GGGTTTTTTCGTAGCACCTCAT | |
| | | GAAAATACCCAAATGACCTTTT | |
| | | CAACCAACCCAGTAATAAGGAC | |
| | | ATTAGGAAATATTGGCCTTAAG | |
| | | AGGTGCAA | |

SEQ ID NO: 392

| | | | |
|---|---|---|---|
| 346498 | SCAFFOLD | CACAGAGGAGAGATAAGGTGTG | G/A |
| | 198487_785 | AATAAGGAAATTTTATTATACT | |
| | | GACATGTGGTGAAAATCTCACT | |
| | | TTACAAAGTATTACAGTTAATA | |
| | | AATGTGTTTGCCATGATGACAG | |
| | | ATTTCCTCCTAAGTGATAGAAA | |
| | | AAGAAACACTGAAAAGTTGAAC | |
| | | ACTTGATGATTCAGAAGTGATA | |
| | | ATCTTTGGAAAAACTGACCACC | |
| | | ACATTACATATTTTATTTAGCG | |
| | | ATGGAGTTTAGACACTTGCATT | |
| | | GCAAGACA | |
| | | TGAAGTACAGTGTCCAAAACAA | |
| | | GGCACCTAGAACTCTCATCATA | |
| | | CTCTGCAAAAGACATCATTCCT | |
| | | AATACATTTTAAGAAAGACACA | |
| | | AATGAACTAAAAAGTATCCAAT | |
| | | GAGGTGAAACCTGAAAGCTGTG | |
| | | ACAGAGAAAAACACTGTCAGAA | |
| | | TGGCTGATCTCCTGCCTGTAGG | |
| | | GCAGTAGAATTAGAAGAAGCAA | |
| | | CTTATAGGTCTTCAAATATCTA | |
| | | AAGGATCAACATGCTTATGAGA | |
| | | AATTAGTG | |

SEQ ID NO: 393

| | | | |
|---|---|---|---|
| 347168 | SCAFFOLD | ATTGTCCTCTCCAGCGATTCTG | G/A |
| | 130253_23585 | AAAATGTCAGGTCTTGTCACAG | |
| | | AAGCGTTTGGCATGTGTACTGA | |
| | | ATTCTGTAGGGAGGGGTCCGTG | |
| | | GTGAACCCTGTGCTAAGGAGTC | |
| | | TTACTGGAAATCAGTGCTGGGA | |
| | | AACATGACATCACTTTGTCGTG | |
| | | TGGAAAACGTACTTAGATGTGT | |
| | | GGCAGGTTCTTCTGAACCTGTG | |
| | | GTTGTCTATCCTGGCTGCCGAT | |
| | | TTAAAACCAGCAGAGGTTTTAA | |
| | | AATGTAGC | |
| | | AACTTAGGAGATGGCCAAAAAA | |
| | | ATTTTTTTAATTAAAAAAAAGT | |
| | | CATGGACTTGTATCCCACCTGT | |
| | | GATTAAGTGAAGCCCAATGTCT | |
| | | AGGATGGGACTTGATTTAACTG | |
| | | TAAAGTCTTCTCAGGCAGCTTT | |
| | | GATAACACTGCAAGTGTTGAGG | |
| | | ACCACTCCTCTACACTGAATTG | |
| | | TCCCCAGGCTTTTGCCTTTAGG | |
| | | ATTTTCAGGGTCTGTTTTTTGG | |
| | | TGGAACCTCAAGCAGTGACGAT | |
| | | TCCAGAAG | |

SEQ ID NO: 394

| | | | |
|---|---|---|---|
| 347176 | SCAFFOLD | TGTTATCATAAAGAACTAAGTT | A/G |
| | 133267_4665 | TATAACTACTTACTCAGACAAA | |
| | | GGATGAACCCTCATTTAGGTTT | |
| | | CAGTTGATTGCAGAGTAGGCCA | |
| | | TCAATAAAAGAAGACAAAATAA | |
| | | TTCAACAGCAGGAAAAATAAAA | |
| | | TTTTATGCCTCTAGCCTCAAAC | |
| | | TTCCTCATTAGTGGAAATTCTT | |
| | | TTAAAATACCATGAAGTATTCT | |
| | | CATTGCCTTAAAAATGTAATTT | |
| | | TTAAAGAATATTGTGAGGAAGA | |
| | | AAAATGCC | |
| | | CAATAGAGCTGAGAAAGGTTTG | |
| | | TGTATAATTGTGACTTTTTTCC | |
| | | TGGGCCTCAGGCCCATAAATGC | |
| | | CTCTCCTTCCCAGCTAGCACAG | |
| | | TGGTAAAGAACCGCCTGCCAAT | |
| | | ACAGGAGGTGCAAGTGACACGG | |
| | | GTTCGATCCCTAGGTTGGGAAG | |
| | | ATCTCCTGGAGGAGGAAACGGC | |
| | | CACCTGCTCCAGTGTTGCTGGG | |
| | | AAAATTCCATACACAGAGGAGC | |
| | | CTGGTGGGCTGCAGTCCATGGG | |
| | | GTCACAAA | |

SEQ ID NO: 395

| | | | |
|---|---|---|---|
| 347369 | SCAFFOLD | TAACCACTCAGACATAGATTGG | T/C |
| | 200949_5881 | AGGTTTCAAAGACCCCCTCATT | |
| | | GGACTTGATTAATTTTTTTAAA | |
| | | GCAGCTAACAGAACTTACAGAA | |
| | | ATATTTACTTTGAGATCACTGA | |
| | | AGTGAAGTGAAGTGAAAGTCAC | |
| | | TCAGTCACACCCAACTCTTTGC | |
| | | GACCCCATGGACTATATAGTAC | |
| | | ATGGAATTCTTTAGGCCAGAAT | |
| | | ATTGAAGTAGGTAGCCTTTCCC | |
| | | TTCTCCAAGGGATCTTCCTAAC | |
| | | ATAGGGAT | |
| | | GAACAGGGGTCTCCTGTATCGC | |
| | | AGGCAGATTCTTTACCAGGTGA | |
| | | GCCACCAGGGAAACCCTTTGGA | |
| | | GAAGACTATAAAATTATATAAT | |
| | | TTAGGAACAAATGGAAAAGGTG | |
| | | CATAGGGCAAGGTATGGTAAAA | |
| | | CCTACTCTGAGCACACCACTCT | |
| | | TCCAAATCTCCACATGTTTACC | |
| | | AACCCAGAAGCTCTCCAAGCCT | |
| | | CTCCCTTTGGGTTATTTATGGT | |
| | | GGTTTCTTATGTGTGTGTGTGT | |
| | | GTTAGTGG | |

SEQ ID NO: 396

| | | | |
|---|---|---|---|
| 347617 | SCAFFOLD | GTTCATGATTGGGAGAGTGAGT | T/C |
| | 280066_1124 | GACTTTGTGAGACAAATACTTT | |
| | | TTTCCTAAGGTAAGAATATGTT | |
| | | GTCTTATTTTCCTGTAGATGGG | |
| | | AGAAGAACTCACATTTGAAATT | |
| | | CTTAATGTACTGGAATTTTCTA | |
| | | GGTAAGCTATCTTTTTGATCTG | |
| | | TATGTGAATTTTCCAGGGCTCT | |
| | | TCTTTGACTATTCAACTGAGAT | |
| | | TTGGCTGTAAATGAAAACCTAA | |
| | | TAACAGAATCCTGAAATTCTAG | |
| | | TTAGCTTC | |
| | | ATACACCCTTGCATCTCAGTTC | |
| | | AGTTCTTGCTTCTCTAGGCCTC | |
| | | ATTATAATAATTGTTTTAATTG | |
| | | GAATACTCTTCTCTGAGGCTTG | |
| | | CTGCTGCTGCTAAGTCGCTTCA | |
| | | GTCGTGTCCAACTCTGTGCGAC | |
| | | CCCATAGACGGAAGGCCACCAG | |
| | | GCTCCCCCGTCCCTGGGATTCT | |
| | | CCAGACAAGAACACTGGAGTGG | |
| | | GTTGCCATTTCCTTCTCCAATG | |
| | | CATGAAAGTGAAAAGTGAAAGG | |
| | | GAAGTCAC | |

SEQ ID NO: 397

| | | | |
|---|---|---|---|
| 347802 | SCAFFOLD | GGTGGATTCTTTACCAGCTGAG | G/A |
| | 45275_15406 | CCACCAGGGAAGTCAGCCTATC | |
| | | CCTTCTCCAGGGGATCTTCCCA | |
| | | ACCCAGGAATCAAGCCCGGGTC | |
| | | TCCTGCATTGCAGGCAGATTCT | |
| | | TTACCAACTGAGCTCTCAGGGA | |
| | | AGCCCATAAAGGATTAAGGCAT | |
| | | ATATTAATAATACTCAGGAACT | |
| | | ATGCAAAAACCAGGAAATAATG | |
| | | CCTGTCCTCACCAGCCACTGCA | |
| | | GAAAACCTAGTAACTCACAGAG | |
| | | TGTTGAGTA | |
| | | AGTACTAGAGGGATTTTGCTAT | |
| | | AGTGGTGGGCAAAGTTAGCCTT | |
| | | AAATGCTGCTCTGGCTCCACCT | |
| | | AACAAAACTTAAATGCAAGACA | |
| | | CAACAGGATCAAATAGGTTTCA | |
| | | AGTAATGTAATTACATATAAGA | |
| | | ACAAAGCTCAAAGTAGTCACAG | |
| | | GAATAAAATGTATCTAGGACAC | |
| | | AGCAAAGTGAAATTCAAAATCA | |
| | | AAAGTGTCACACAATCAAAAAT | |
| | | TATTGAGCCTGCAAATTTGCAA | |
| | | GAAAATAC | |

SEQ ID NO: 398

| | | | |
|---|---|---|---|
| 347981 | IBISS4snp291 | GTTTGAATTCCGTTTGTGCCTA | C/T |
| | | TTCCCCAGCCCCAATCCTTACC | |
| | | GATTGGTATTTTCTCCTTTTGC | |
| | | ACATGTCCCCTGCCTGCTCTCA | |
| | | CTACCCAAAGAG | |

SEQ ID NO: 399

| | | | |
|---|---|---|---|
| 347988 | IBISS4snp414 | TTGTGGGTATTTGCCTCATTCC | T/G |
| | | ATCCCCAAGCTTTGCAGCTAGA | |
| | | CAGTGAATTCAAAACTACGTTC | |
| | | TAAGGCGTGTCCTGTAATGGAG | |
| | | TCATTGGTTGAC | |

SEQ ID NO: 400

| | | | |
|---|---|---|---|
| 348068 | SCAFFOLD | ACAGGGAATTTGAGGATACAAA | T/A |
| | 110535_883 | TACTCTTAGCTTGATCTAGATA | |
| | | CGGGTTCCAGATGGACTAGTCC | |
| | | CTCAAAATCTTCATCCTATCAG | |
| | | GAGCGGGATAGCTGGAAAAAGG | |
| | | CCAGATGAGGACCTCTAAAGCA | |
| | | CAAGGCCCAGACTAGGGTTCCC | |
| | | GCCTGGATAACTCTAAGGAGAG | |
| | | AGTAACTCTCTAGTCAGTCCTG | |
| | | GCAGGTATTTCCAGGACGCCAT | |
| | | CCCTCCCTAGTCCTCTTCAGAT | |
| | | CTTCACAC | |
| | | CTCTCTAAACTATCCTAGACAC | |
| | | TGCCCTGGCTTCACTCAAATCT | |
| | | CTCTTCCAGTCTAAACCATTTT | |
| | | ATGGGTGCCAGTTTGTCTACTA | |
| | | GATATTTTCTTGCCTATACTTC | |
| | | CCTGCAAACTTAAACAGCAAGC | |
| | | ATCGATTCCAGGATATCCCAAG | |
| | | GACTACGTTAATTAATTCAACC | |
| | | AATCAATAAATATTAATTGAGC | |
| | | GCCTTTTATGTACTAGTAGTTT | |
| | | TCAGTTAGAGTTACAGTGGTGA | |
| | | ATAAAGCA | |

SEQ ID NO: 401

| | | | |
|---|---|---|---|
| 348126 | SCAFFOLD | GTTAGAAGTAACCCAGAGGTAA | C/T |
| | 130001_85195 | TGGCTTGCACACACAGGAATGC | |
| | | CCGCCTCCTCAGGTTCTTTCTC | |
| | | CTCTGCCTTAGGAAAGAGGACC | |
| | | TGCTCAGAGAAGTCTGAGCTCA | |
| | | ACAACCTTTTCTACTTGATACA | |
| | | ATCCATCTCCTCATTCTGAGTT | |
| | | CAGCTGTTGTGATTGCATCCTT | |
| | | ATATACTCATTCATTTCCTTTG | |
| | | CTCCCAACTTTACATTCAGTCA | |
| | | GCCTCACAGAAATCCACTAAAC | |
| | | TTAATAAA | |
| | | GCTGCCTACAGGCTCTATGTTA | |
| | | TCTTTGAGTGATATGATTTGTG | |
| | | CAAATTTTTACGTAAGAGAATT | |
| | | CCCCTTTCCTTCCAATTTCTAT | |
| | | TAGGTCTGTTAATGTGTCTAAA | |
| | | ACATTTCATTAGATGACTGTTT | |
| | | CTATTTTTCCTTTTATATAAAA | |
| | | CAAATTGTCCTTGCCAGGTTAA | |
| | | GGAATCAAGGTGAATCCTTTAT | |
| | | AACTGTTAATACAAAACACAGC | |
| | | ACCAGAGGAAGGCCCCAAAAAC | |
| | | CTAGAAAGG | |

SEQ ID NO: 402

| | | | |
|---|---|---|---|
| 348185 | SCAFFOLD | ATGCATGAAAGTGAAAAGTGAA | G/C |
| | 146214_10543 | AGTGAAGTCGCTCAGTCGTGTC | |
| | | CGACTCTTAGCGACTCCATGGA | |
| | | CTGCAGCCTACCAGGCTCCTCC | |
| | | ATCCATGGGATTTTTCCAGGAA | |
| | | AGAGTATATTCTTCCTCAAAAG | |
| | | CAAGACCTAACCACAGCTGCAA | |
| | | ATGTGTTAAGAGAAGATGTGAA | |
| | | GTAGTGGTTAAAATCAGAACCT | |
| | | GGATTCAGGACATTTCATCCCA | |
| | | GCTCTACCTACCACTTTCTAAC | |
| | | TGCATGGC | |
| | | TTGAGCAAGTTTTTTAAAACCA | |
| | | CACTGCCCTTCAGTCTCTTTGC | |
| | | CTGCAACATGGAGTTAAATGAA | |
| | | GTTCTTTGGATTGTTGATTATA | |
| | | TGAGGCAAAGCACATAAAAGAC | |
| | | TACCTGATAAAGAAATGCTAAG | |
| | | TGCAGAGTCTTATTTCCATGTG | |
| | | AGAAGTCTTATTACTTCCCTAT | |
| | | TTCTGTAGATCAGACGTCAAAG | |
| | | CCACATTTACACATCCAGGAAT | |
| | | TACAATCCAGACCTGAACACAG | |
| | | TTCTTTTT | |

SEQ ID NO: 403

| | | | |
|---|---|---|---|
| 348246 | SCAFFOLD | TTTAGTCATACTCTAATAGTAT | C/T |
| | 1654_783 | GACAAAAGTACTCTAATCCATT | |
| | | GAGTAGACAGAATGCAGAGTAT | |
| | | TCAATAGTTTTGAGGAAGTGTT | |
| | | TCTATTCCACATTAGATTTCTC | |
| | | AAATTACTGAGCTTTTCATGTA | |
| | | ATCTCTTATCACAAAAAAGTTA | |
| | | CTTACTATTGTTGCATTTATAA | |
| | | TATGTGTTTAATTGAGTGGGGA | |
| | | TACAGAATAGCTTCTCCAGGTT | |
| | | TAAGATTGCCAACTCTGCAGAA | |
| | | CTAGTATC | |
| | | TGGGCACCTGTTATTAAGATGA | |
| | | CATTTAGGAGATTAAGTTTTTT | |
| | | TTATTAATTAGGTTTTTATTAA | |
| | | ATCTATCCATTTGAGTCTCTAC | |
| | | CCCTTCACCAATTAATAAGTAA | |
| | | TCTGGAGAATCCCTTTCAGAAG | |
| | | GTAAATAAAAACAGGAATAATC | |
| | | AAGAGTCTGCAGTATCTCCACA | |
| | | CTTAGGTTTCAATGAAAATCTA | |
| | | AAAGGAGTTGAATTTACATTTG | |
| | | AAATAGACATATTTTAGGGTCT | |
| | | CCTCCTAA | |

SEQ ID NO: 404

| | | | |
|---|---|---|---|
| 348275 | SCAFFOLD | TGAAAATTCTTCCCATTTTGAC | G/T |
| | 181062_3459 | ATAAATCTTGAAAATAATAATT | |
| | | TACTGTTTGTAACCAAGTCAGC | |
| | | TAAAACCTAATTGGCTCAAACT | |
| | | CTATAAGTCTTGAGGTATTTTC | |
| | | ATTGTCTTTCCTTTGTACTGCC | |
| | | TTAACTCAGATTTAATATACAG | |
| | | GTGAAGATATTTCACCTGTGTT | |
| | | TGCCAATCTATTCCAAGATGCC | |
| | | AATTATAACATAAAAAACACAA | |
| | | TGAGTCTCTATGCATGAAGTAG | |
| | | ATAAGGTT | |
| | | ATAATGTGCCAGGCATTTCATC | |
| | | ATTTATGGGTTATAGATGGAGA | |
| | | TTTTCATTAAGCATTATTGAAA | |
| | | TTACCATAACATACATGAGTAC | |
| | | ATATTCCAAAAAAGAATTTCTA | |
| | | TTAGCAATGTAGAGAAATGGTA | |
| | | CCTGTTGATAAAACAAACATAG | |
| | | TATTTGTTCACTTGGTGCTTTC | |
| | | AATTACTTAAACAAACTCAAAA | |
| | | AGAGGAGGAAAAAAATGAAAAG | |
| | | TATTTTAATTGGAAACATCAAA | |
| | | CAATTTGA | |

SEQ ID NO: 405

| | | | |
|---|---|---|---|
| 348325 | SCAFFOLD | CATTAAACAAAAGTGTAATGTG | G/A |
| | 200299_9268 | TTGAGTAACATTCTCGTACATA | |
| | | TCATCACCTAATGTTGATTTTT | |
| | | GATGAAATTTAGGGATTGCTCA | |
| | | TGGATTTGAACAGAAAGGGAAC | |
| | | CCTTTGGAACTGACTGGCAGAG | |
| | | CTAGTATGAGAATTGATCAACA | |
| | | GGAGGTTCTGACCCAAGATACT | |
| | | ATAGAAAGGGTTGGAGAGAATT | |
| | | GTATAAATAATTTTCTGGTTTA | |
| | | AATCTATCACAGTGCTATCACT | |
| | | GCTTTCCT | |
| | | GTAGCAAATGAGTTTGATTTGA | |
| | | TTCAGTCAAAATTTCTGGTATA | |
| | | ATACAAATGAACATTTATAAAC | |
| | | CAAATGCAAAAAAGCCTCAGTA | |
| | | ATAGGAATGAGAATTAGTTTGA | |
| | | GCAGTAGAGAAGTATTCTCAGT | |
| | | GCTGAGAGGCTATGATTTCGTG | |
| | | AAATTAAAGATCTGATTTTAGT | |
| | | GCATGCAAAATAGGCAGTTTAA | |
| | | ATATAGGTCACCTTTTCTACCC | |
| | | AGATAAATTACTTCTGGTAAAC | |
| | | ATAAGTAT | |

SEQ ID NO: 406

| | | | |
|---|---|---|---|
| 348415 | SCAFFOLD | TTCTGATACTTAATGGCCCCTT | G/A |
| | 230074_9581 | GGTCCCTATCATCACCAATTAG | |
| | | ACTACAAGAAGAGTCAAGTGTG | |
| | | GAAAGTCTCTTAACATATAAAA | |
| | | TGCTATTTCCCTGATTTCTTAG | |
| | | GCTTATTTGGCATAAACTGAAA | |
| | | TTTTGCCCATCACAGAAATAAA | |
| | | AATGTCTAGAAAGGTCCACACA | |
| | | CATAGATCTTTTAGTGTCTAAA | |
| | | ATAATTGCATCCTTTTGTTGTG | |
| | | TTTCCTGGAACAGCTAAGCTTT | |
| | | ACACTTAC | |
| | | ATCTTTGGTTTCTTTGAGCTCT | |
| | | TGGACCTTTAGAAGTGAACATG | |
| | | CTTGGGCAGGAATAATCAACAA | |
| | | GCCATGTCAGAGAAGAAAAGAG | |
| | | CAGGACAAGATGAGAAGGCCTT | |
| | | TTCTTTATATGCTTACATCCCA | |
| | | ACCATTGTCCTCTGTGATCTTT | |
| | | CATCTGTGTTTTCAACCATGGG | |
| | | GCGACTGCTGATAAATTCTAAC | |
| | | AGAGTCCTCTACTTGGATAACC | |
| | | AGCTCCAGAGAAAATCCAGGTT | |
| | | GTTTAAAAT | |

SEQ ID NO: 407

| | | | |
|---|---|---|---|
| 348459 | SCAFFOLD | ATACATGAGCTATTTCAGAGGT | A/G |
| | 245593_3874 | GGAAATGGCAATGTTTGATGAC | |
| | | TGAGCATGGCAGCAGTGGGAAC | |
| | | TAAAGGGTCCAAGGAGGACAAC | |
| | | CAAATTAGGTGTAAAGATCATT | |
| | | ACTAATGATCCCTCAGACCTCT | |
| | | CTGATAACTTGGAAAGGGGTCA | |
| | | GCTCCCATACACTTGATCATTT | |
| | | TTACTATTCAGTTTATCTCTGT | |
| | | TTCAATAGGGATTTACTCATAC | |
| | | TCTATACATAGCTGCCAAACTA | |
| | | TTCACTTC | |
| | | GGGATGACTCTTTTTGTCTAAC | |
| | | ACCATAGAATCATCAGAAGAAG | |
| | | ACATAAAAGTGATCCTTATTTG | |
| | | TAGGTTTAGAAAAAAGATTTGC | |
| | | TGATATATTTCTATCTGTTTTG | |
| | | ATTAAATACTCCAAAACTGTTT | |
| | | ACATACACATTTTAAAAGGTGA | |
| | | TTTTGTATCTCCTTTTCTATAT | |
| | | GTCAAATAAATATTTGGTTTCA | |
| | | GAGATAAGTTTTAGTTATAATT | |
| | | TACTTGCATGTTTTCTATGAAC | |
| | | ACTGAAA | |

SEQ ID NO: 408

| | | | |
|---|---|---|---|
| 348572 | SCAFFOLD | TGAAGCTCCTTTCAGCAGGACT | C/T |
| | 285028_33789 | TCAACCTAATAGCAAATAATCA | |
| | | CATCCCTTCTGGTGATGTTTAT | |
| | | GAACTAGAGAATGGTTCCAATT | |
| | | GGTCAGTTTCATGCAGGTCTGA | |
| | | ATATACACATTTTGATTGAATT | |
| | | TGCTCAAAAGAAGATAGCTGCA | |
| | | CTGTTAATCATATTTATTAGTT | |
| | | GATCTTAAGAATTCATGCATTT | |
| | | AATTGTTCTAGAATTAGAGATA | |
| | | AACCTTGGAATTATGACTGTGA | |
| | | GAAATGGC | |
| | | AAGAGTTTCATTGGGGATGACA | |
| | | GAAGGCAATGCTGAGCAAGAAG | |
| | | GAGGTTTGAAATCCTGCATTAA | |
| | | TTCTAGAGTTCATCAACCTATG | |
| | | ACTATGGAAATCTCGATATATT | |
| | | TGAATTGCAAAAATTTATAAAA | |
| | | AACTCTTAAATACTTTTTGTCC | |
| | | TTCTGTTTTCTCCTTCAATATA | |
| | | AGCCTACAAAATTATAAGGTAC | |
| | | TTAAGTGTTGGGACCATGTCTC | |
| | | TGAATCTTTAATAACAAACACA | |
| | | CTCTCATA | |

SEQ ID NO: 409

| | | | |
|---|---|---|---|
| 348929 | SCAFFOLD | ATAGATGGGACCCCAAAACAGA | C/T |
| | 10954_11613 | ACAGACATAGTTTGGAGGGTGG | |
| | | AGGGGAACAGCGAAATCCAGAT | |
| | | AAAGCCTGGAGCTGAATTTAAA | |
| | | GCAAGGCGCCTGTGTTCATTTC | |
| | | TTCATTTTGGCAAGATGGCAAC | |
| | | CTTAGGAAGAGCTGGCTGAGGG | |
| | | CAGGTGCAAGTTCTCCATGCTG | |
| | | TTTTTGTCACTTTTCGTTCCAT | |
| | | CTAAAATCATCCCAAAATCAAA | |
| | | TGTTAGAATTTGTGATGGTATG | |
| | | GGGAAATA | |
| | | AGTAAGTCCCCACTTATGAGTG | |
| | | AGTTCCCTTCCAAAAGCACATT | |
| | | CATAAGTCCCATTTTTTCGTAA | |
| | | TTCCAACAAAGTTAGCCTAGTA | |
| | | CGCAACTTTTACAGATAATGCC | |
| | | AGACAGGTGAATTAGCTTATGT | |
| | | GATTGGACATGTGAACACAAGT | |
| | | TTGAATCTTTTTTGTTTGTTTG | |
| | | TTTTTTACTTATTTATTTCTGG | |
| | | CTGAGCTGGGTCTTTGTTGCTG | |
| | | CACTTGGGCTTTCCTCCAGTTG | |
| | | TGGCAAGC | |

SEQ ID NO: 410

| | | | |
|---|---|---|---|
| 348963 | SCAFFOLD | ACAGGCCCCTCCCTCCCACCTG | T/C |
| | 120012_10659 | CCCCTCCCTTCCAGCCACCCCT | |
| | | CTTAGGCTGTCACGGAGCCCCA | |
| | | GCCTGGGCTTCTCTGTGTCATG | |
| | | CGGCAAATTCTCACTGGCTGGC | |
| | | TGTTTTACAGATAGCAGTGTGT | |
| | | ATGTGTCATCGTTACTCTCTCA | |
| | | AATCGGACCAAAATTTATTCAG | |
| | | CTATTCCCCTGTTGATGAACAA | |
| | | TTAAATACTTTCTAGTTTTATA | |
| | | AACACTGTAGTACAAACATCTC | |
| | | CATATGGA | |
| | | TGATTGTGAGCATGTGTAAAGT | |
| | | ATATTGATAAGACAGATTTTTA | |
| | | GATGGTCAAAGGAGATTCTAAT | |
| | | TTGAGGTTTGAGAGACAGTTCC | |
| | | AAGTTTCCTCTCTCCTTCCTTC | |
| | | CTTCCTTCCCCTCTCATTCTTG | |
| | | CTACAGTTTTGAGAATACTGTG | |
| | | TATTATCACTTGTTTTCATGCT | |
| | | TGTCAATTTTAGTAGGTGAAAA | |
| | | ATAATTTACTGATTATTTAATC | |
| | | TGAAGATTTTAAATTGTGGTTC | |
| | | AGGTTGTA | |

SEQ ID NO: 411

| | | | |
|---|---|---|---|
| 348968 | SCAFFOLD | GAATATTTGCAACCACGTAAAA | C/A |
| | 121110_8034 | TTTTGTATAAATCAATTATATA | |
| | | ATGTGTGATTCTAATATCATTT | |
| | | TCCTTTTATCTTATTTGCTAAC | |
| | | TTCCCACTTTCCAGATATTCAG | |
| | | AGGGTAAAGCATGCTGATTAAG | |
| | | AATCTAGTCTCTGAACTCAACT | |
| | | CCCAGAGTCCAGATTCCAGCTC | |
| | | TCCTCTGAGCTCTGTGACTTAG | |
| | | GACAAATTATTTAATCTCTGTG | |
| | | CACCTCAGTTTCCTCATATGTA | |
| | | AAATGGGA | |
| | | TGATGACACCTCCATCATAGGA | |
| | | TATTGAACATTAAAACAAGTCA | |
| | | ACCCCCTTAAACATGTCAAAGA | |
| | | ATGTCGGATACACAGTAGAAGG | |
| | | CACAAAATTTTTAGCCATTGTT | |
| | | AAATATATGTGCTTATTTTCTA | |
| | | AAAAATATGTCATATTCTAGGC | |
| | | ACTTGTTACAGACAGCAAAACT | |
| | | TTGTGGAAACTGCTTGTAGTCT | |
| | | TTGTGTAAATCTAATTCATCCA | |
| | | TTTTAAAGCTCAAGTGTAATCC | |
| | | ATAGCTTG | |

SEQ ID NO: 412

| | | | |
|---|---|---|---|
| 349106 | SCAFFOLD | GAGTTGGACACAACTGAGAGAC | C/T |
| | 170646_5902 | TTCACTTTCAAGCTTTTAAAAT | |
| | | TATTTGATAGGGACTGGTGTTT | |
| | | TTAATTCTGCTTTGAGGCACCC | |
| | | TGAATATTCTCACCTATAAATT | |
| | | CCTGGAAGTAGTCAGATTAGCA | |
| | | AGGAAACATTCCTTTGTAAATC | |
| | | ACCACATTTGGTGGTACTTATC | |
| | | CTGTGGAATATTAGCCAATACA | |
| | | CTGAGTGCTATTTTTACTAGCA | |
| | | TTAAATTCATACATATTATTTC | |
| | | ACCACTGG | |
| | | GCTAGTGGTAAAGAATCTGCCT | |
| | | GATAACACAGGAGATGCAAGTG | |
| | | ATGCAGGTTCGATCTCTGTGCA | |
| | | GGAGGATCCCCTGAGGAAGGAA | |
| | | ATAGCAACCCACTCCAGTATTC | |
| | | TTGCCCAGAAAATTTCTTAAAT | |
| | | AGAGAAGCCTGGTGGGATATAG | |
| | | TCCACAGGGTTGCAAAGAGTCA | |
| | | GACACGACTGAGCACACACACA | |
| | | TATTTCACCAGAACAATGAAAA | |
| | | ATAATTATTTCTTTACTTGTAT | |
| | | TTATCCT | |

SEQ ID NO: 413

| | | | |
|---|---|---|---|
| 349116 | SCAFFOLD | TTGCTGAAAGAGGAGTTAGCTG | C/T |
| | 1747_667 | GCATATTAATATATTATTCCAG | |
| | | ACAACAGAATGTGAGTGAATAA | |
| | | ACTAGAAGATTTAAAATGAGAG | |
| | | TGCAGAGGAGACAATGCATATG | |
| | | AAAGAAACTGGAGATTTAGAAG | |
| | | CATGGAAATGGCATGACACTTT | |
| | | GAATGTGGAGGAAGAATGGAGA | |
| | | GAAGTTAAATAAATCTGTATCT | |
| | | GTTTATTGAAACTCCTTAAGAG | |
| | | AAAAGGAATGATTAGCAAATTA | |
| | | CTAAGAGG | |
| | | GGCTTTTCTCTTTGCTAGATTT | |
| | | AACCAGGAATTTGTGTGTGTGT | |
| | | TTGTATGTGTGCACTGTGTTGT | |
| | | GCGTTTCATTAATATTCCTCTT | |
| | | ATTTTTATTATTTATCCTTCTC | |
| | | ATTCACTGTTATGCTGAACATA | |
| | | GCCAGAAAATGAGGTCAACTCA | |
| | | ACATTTGCTTTTGCTCAAGGCT | |
| | | ACATCAGGCAAATGAATTCTTT | |
| | | GTCCAATTCAGAAGAGAAATAA | |
| | | ATCAATTACCCTTGCTGAAGGG | |
| | | TGGCTATC | |

SEQ ID NO: 414

| | | | |
|---|---|---|---|
| 349117 | SCAFFOLD | AAAGGAATGATTAGCAAATTAC | A/G |
| | 1747_446 | TAAGAGGCGGCTTTTCTCTTTG | |
| | | CTAGATTTAACCAGGAATTTGT | |
| | | GTGTGTGTTTGTATGTGTGCAC | |
| | | TGTGTTGTGCGTTTCATTAATA | |
| | | TTCCTCTTATTTTTATTATTTA | |
| | | TCCTTCTCATTCACTGTTATGC | |
| | | TGAACATAGCCAGAAAATGAGG | |
| | | TCAACTCAACATTTGCTTTTGC | |
| | | TCAAGGCTACATCAGGCAAATG | |
| | | AATTCTTTGTCCAATTCAGAAG | |
| | | AGAAATAA | |
| | | TCAATTACCCTTGCTGAAGGGT | |
| | | GGCTATCTTGAGTGCTTTTAGG | |
| | | AAAAACTCAGGTCATTCTAAAG | |
| | | AAATGTCTGAAATATCTGCAAG | |
| | | AGTTAAGAGCAAGATAAGCACT | |
| | | AATTTACCATCTCCTTCATCCC | |
| | | CATAAGGAGGTACTTAAAAGAG | |
| | | GACAAAATGCATCACTAGGTAT | |
| | | TGTCAGGGTCAAACTAAGACTT | |
| | | CAGTATTTCTTTGAATAAAAGT | |
| | | ACTATGCTCCACTGCCTAAGAT | |
| | | GAAGAGTG | |

SEQ ID NO: 415

| | | | |
|---|---|---|---|
| 349235 | SCAFFOLD | CTACAATCCATGGGGGTCGCGA | G/A |
| | 210732_8597 | AGAGTAGGACATGATTCAGTGA | |
| | | CTGAGCACATGCACATACAATA | |
| | | TTATATATTTATCTTTATACCA | |
| | | GTAAGTCATAAATGTAACCTGT | |
| | | AACATTTGTTGTAGCCCTGGGT | |
| | | AAGCTAAGATGAATTCTTTGAG | |
| | | GAGTCCAGTTATCTGAAGTTCA | |
| | | AGGCACACGAAGACATTGTTCT | |
| | | AACACTGTCAGGAATCACAGAT | |
| | | GTGAAGCAATTAGTTCTATCTT | |
| | | GGGAAGTA | |
| | | GAAAGGCTTTGCAGATTGTTGT | |
| | | TGCTGTTTAGGCTTTGCAGAGC | |
| | | TGATATTTACATCAGTTATTTG | |
| | | AGGATAAATATGTTTTCTACAG | |
| | | GTGAAGATAGTGGGAAGGGCAT | |
| | | TTCTGGGAGAAAAAGCAGCATA | |
| | | AACCAAGGAATAGGGTTATGGA | |
| | | AAGAGGGTGTTGGATCTTGGCA | |
| | | GTGGTCAATAACCAATAATGCC | |
| | | TTTCTCAGTATAAAATCATAAG | |
| | | GGAAAATTCTGTTACTTCTATT | |
| | | GCTAAATC | |

SEQ ID NO: 416

| | | | |
|---|---|---|---|
| 349299 | SCAFFOLD | ATTCTTCTTTGGTCATGATGCT | A/C |
| | 230591_4636 | GAGTGTGGCCTGAAGATGATGG | |
| | | AAATAGTGAAGAACGGATCTGA | |
| | | AAACTGTAACAGATCCAAGTTG | |
| | | CATGGTCAACTAAAAAGTAAGA | |
| | | AGTAGAGCTTTTCTTCAGCTAC | |
| | | CTAATGCTGTGTTATGCTGTAT | |
| | | ATGCTTTCCTAATTAATCTCTT | |
| | | ACAAGACTTGATGGTTTCCAAA | |
| | | ATCCCATCTGATAACTGCTCTG | |
| | | TCAGTGACAGACAGTATCTTTG | |
| | | GATAAGTG | |
| | | AATGGCCTATTAGCCAAATTGA | |
| | | ATGAAAGTGTGACAGTTTATTA | |
| | | ATAATGACATTTAAATGATATT | |
| | | TCCACTCAGAGAACTTTGTTGC | |
| | | CTGTATCAGGATGTAGGTTATA | |
| | | TGAACTAGCAAGTAATCTTAAA | |
| | | TTCTAAGTGGCATAAAACTACC | |
| | | CAGATTCATTTCCTGCTCCTAC | |
| | | TACATATCTCCAGAGCTGAGAT | |
| | | GGAGGGAATGAGTTTGGGCAGG | |
| | | GGGCGAGAATTTGCTCATTGTT | |
| | | GTTATTAC | |

SEQ ID NO: 417

| | | | |
|---|---|---|---|
| 349323 | SCAFFOLD | CTGAAACTTTGCCAGGCCTTGG | G/C |
| | 240108_5381 | CCCGCAAACGTCTTCCACCCTA | |
| | | CCCAGCGCTGACAGACAGCACA | |
| | | ATCCGGAGCCCATAAAGATTAG | |
| | | AAATGTTGTATTAGTCAGTGAC | |
| | | CCTTCCCAATTAAAATACTTAG | |
| | | TTCAGGGTTCTTCACACAATGC | |
| | | TTAAAGTACTCTCTGTTCAACA | |
| | | AAGGCTTGTTGACTGAATTTAC | |
| | | AGCCTGTGCTGTAACATGAACT | |
| | | GTAGACCGAAGTCCAAATTACA | |
| | | ACACATGA | |
| | | CTCTATGGCAAGAAAGAGTGAA | |
| | | AAAATAAAGCAACCCTGGACAC | |
| | | ACGCCAGATGACGGCCGGGCCC | |
| | | ATGAGAAACTCCAGAGAGAGAA | |
| | | AAACGCACTAAACAGAAATAGC | |
| | | AGTGCAGGCACCACATCCTTGC | |
| | | TCTCTGATCCTTTGTGAACTAT | |
| | | GATCGCTCCCCTTCACAAAATT | |
| | | ATAAGCCTCTCGAGAGCAGGGT | |
| | | ACGATGAAGTGTCAAGTGCTGG | |
| | | GTGCCCACCTTTCCTGTCAGTC | |
| | | CAGTCATC | |

SEQ ID NO: 418

| | | | |
|---|---|---|---|
| 349456 | SCAFFOLD | GTAGGTGTCCAAAACTATATTA | T/G |
| | 285028_33288 | TTGATTGTTTTATATGTGAGTT | |
| | | GGAGAAAGATGCATAAAAATGT | |
| | | CTAAGCATGCTAGAATTCTCTT | |
| | | TATTTATTTTTTGGCTGCACCA | |
| | | CTCAGCTTCCAGGATCTTAGAT | |
| | | CCCTGACCAGGGGTTGAGCCCA | |
| | | GGCCTTCACCAGTGAACGTGGT | |
| | | GAAAGCACAGAGTCCTTGGGCC | |
| | | ACCAGGGAACTATCTAGAATTC | |
| | | TTCATATAGCCATGATGGAGTA | |
| | | ACTGGTAT | |
| | | AGACTTACCCTCTTATCATGAA | |
| | | GTACTATGAGTAAGTGTGTCAC | |
| | | TTAGTCATGTCTTACTCTTTGT | |
| | | GACCCCGTGGACTGTAGCCCAC | |
| | | CAGGCTCCTTTGTCCAGAGAAT | |
| | | TCTCCAGGCAAGAATACTGGAG | |
| | | TGGGTAGCTATTCCCTTCTCCA | |
| | | GTGGATCTTCCCAACCCACGGA | |
| | | TCAAACCCAGGTCTCCCACATT | |
| | | GCAGATGGATTCTTTACTATCT | |
| | | GAGCCACCAGGAAAGCCCGAGA | |
| | | ATACTGGA | |

SEQ ID NO: 419

| | | | |
|---|---|---|---|
| 349492 | SCAFFOLD | CTCCAATAATGTTTATTCTTCA | T/C |
| | 295327_16712 | AGTTCCAGAAGGGCCTTTTTTT | |
| | | TAAAAAAGCCATTTCACTTTAT | |
| | | TAGAAATCAACGGTGCCCTGTG | |
| | | GGCCACTAGGCTGGTCTCTAAA | |
| | | TGGAACTTCAGGGCGTCTTTAC | |
| | | GTTTAGCAATTTATCAAGGACT | |
| | | ATTGAAGACTAGTGCTAGAGAT | |
| | | GTCGCACTGAGCAAACAAAGCC | |
| | | TCATAGAAACAAAGCTACATAG | |
| | | TTATCTTCTGGGAGTTTTTGTG | |
| | | TGTTCTCA | |
| | | GGTAACATCTCAGGTTTTTGAA | |
| | | GTGTGGGTTGTCCCACACTATC | |
| | | CTAAAGTACAAAAGTCATGAGG | |
| | | ACAAAAATTCCCTTCTGCTGTT | |
| | | GCAGCTGTAGGGTCACCTAGTG | |
| | | CCTTATTTAATCAGCTGATAAA | |
| | | CCAGCATATTCCTCTCCAGCCA | |
| | | TAAAGCAAGATAAGAGCTCAGA | |
| | | AGTCATCATTCCCCTCCCTTGC | |
| | | TTCTCTCTCTACACATGTACCT | |
| | | GTATCTATTATATACTTAACTC | |
| | | AGAGTCTG | |

SEQ ID NO: 420

| | | | |
|---|---|---|---|
| 349564 | SCAFFOLD | AGGGAAATTGGGAAGATTAAGG | A/G |
| | 321782_19330 | ATAGCTTTTTAATCAAGACTCC | |
| | | TAACATTTCCTCTCAAGGCCAG | |
| | | GCACATAGCAACCAACAAGCAA | |
| | | TCATAGCAACAGTGAATGATAA | |
| | | ACATCTATGGAAAATAACAAAA | |
| | | CAAAGAGGCATCAAGCTTTAGT | |
| | | CTAGATTATGTTATGATTCTTT | |
| | | ATGGACAGTTCAGAAGCCATCA | |
| | | AAGCAGTATTTCCTTCAAAGCT | |
| | | GAGAACAGAAGCACGATGTTGG | |
| | | CACACACC | |
| | | TTTATTTCATCGTAAATACACT | |
| | | TGTTAAGTACCAGGTTCGAATC | |
| | | TTTTTGCTTCTCTGAAAATCTG | |
| | | GATAAGTCACCAAATTATTTTG | |
| | | AAATTGAAGGGTATAGAGAGAA | |
| | | GGTAACTAATATACTAAAATAC | |
| | | CTGAGTACAATGGAATGATTTT | |
| | | TGCATTATAGAGAAAAAGCACT | |
| | | TACGTTACCTTAGTGAAAAGTT | |
| | | GCCAGATAGTGACTACTCAGTT | |
| | | TCCAGAATGCTGTCACCTGAGC | |
| | | CTGATTTA | |

SEQ ID NO: 421

| | | | |
|---|---|---|---|
| 349583 | SCAFFOLD | GTCCAGATGCCTTGGCAATCCA | A/G |
| | 35711_6569 | GAATGCAGTCAAGAATTAAGTA | |
| | | GGAAAAAATAATCTGAAAAACT | |
| | | CAGTGATTATTCAAAAGTCTGG | |
| | | TTACTAGTATTTATGTATTTAC | |
| | | TAATACAAGCCCTTGTTTAGCT | |
| | | CTCATGGGTAAAACCTTTCTTT | |
| | | TGTAAGTTTTTAAAAAGTTGAT | |
| | | ATTTGGTTTCTCAATCCACAGT | |
| | | ACAATGGAAAAAGCCTTGAGGG | |
| | | GAGGGCTAGGGTGGAGGAAGAT | |
| | | CTCAGAG | |
| | | CTACAGAATCTGTTACAGTATT | |
| | | ATTACTGGTTATTAGAAAGTAG | |
| | | ATTAAAACCTACCCAAAGTAGG | |
| | | CAATGGAACAAAAACATAGAAA | |
| | | TCAGAATCTATTTAGCAGGATA | |
| | | TCCTATACCTTAGTCATCTTCG | |
| | | GGGAAAATAATCACAGTATATC | |
| | | GCCTGACAAAAACATAGTAACC | |
| | | AAACAAATTTTAAATAAATGTT | |
| | | TGCTATATTAAAATAGAACAAA | |
| | | AAGGCTTAGCGTGTGATTTTTC | |
| | | ATGTTTTT | |

SEQ ID NO: 422

| | | | |
|---|---|---|---|
| 349616 | SCAFFOLD | AGGAGCTCTCCGCTTCCCCACG | T/C |
| | 50203_8741 | TCCTGCCTGTCATCTTCTCAGA | |
| | | AAGGGCTTCCCATCCTTGACTT | |
| | | GTCCGTCTTATGGCTCTGTCCA | |
| | | TCCAGAAGCCACTTTTCTGATC | |
| | | CCCAAGCCCTTGTAATCAACCT | |
| | | TCATCTGCCCTCAGGTATCAGC | |
| | | ACGATCCATCTTTTTACATCGT | |
| | | CTATCATATTCAAATGATCACA | |
| | | AACATACCAGCAAAATGGGTTA | |
| | | ATAGCTCTGACTGCTTATTAGT | |
| | | ATAAAACG | |
| | | TAATGCGGAGACTACATGAGAA | |
| | | GAAGAGAGGCCAAGTTCTTAAT | |
| | | GCCAAAGGGGGATTAATGTATG | |
| | | AATTTCTGCCTATCTTCTCATT | |
| | | TGCTTTCTCGAGAGTCATCACC | |
| | | ATGAATTACAACCACTCTCCTT | |
| | | CCTGACTCAGTACCCTCTAGGG | |
| | | CATCTCTTCATCACAGGAGCCT | |
| | | CCTTTCTTATAGCTTTGTCTTC | |
| | | ATTACTGCCCCAGAGAGGAACA | |
| | | ACAGGAAGAAGAAAGTTAATGC | |
| | | AACTTCTT | |

SEQ ID NO: 423

| | | | |
|---|---|---|---|
| 350226 | SCAFFOLD | TCCAAAGAACACCCAGGGCCAA | A/T |
| | 265287_28176 | TTTCCTTTAGAATGGACTGGTT | |
| | | GGATCTCCTTGCAGTCCAAGGG | |
| | | CCTCTCAAGAGTGTTCTCCAAC | |
| | | ACCACAGTTCAAAAGCATCAAT | |
| | | TCTTCAGCACTCAGCTTTCTTC | |
| | | CCAGTCCAACTCTCACATCCAT | |
| | | ACCTGACCACTGGAAAAACCAT | |
| | | AGCATTGAATAAGTAAGTCATA | |
| | | GCAAATAAAAACTTTGTAGTGA | |
| | | TTCTGTTTTTTCCCATCAGAAT | |
| | | GCTTTTTT | |
| | | AATCTTGGTCAGAATGTTCAAA | |
| | | AGCATCTGAGGCAGAGTTCATT | |
| | | AAAAACATTTTTGTAAATGCTT | |
| | | GTCATTTTGAAGCAGTACAGAA | |
| | | AGTTTAAATTAGTATAAATTTT | |
| | | GTCAGAATTTATAGCTATAACT | |
| | | CACATAAGACTGTTTATCCATT | |
| | | TCCCCTTTTTGTTTAGCAGTGC | |
| | | TATTAATGGTTTCGAAAAATAA | |
| | | CATATTCTCTGTCATAATGTTA | |
| | | TCTTATGTTTTAGTAGATTTTT | |
| | | CTGATGGC | |

SEQ ID NO: 424

| | | | |
|---|---|---|---|
| 350519 | SCAFFOLD | GAAGCCCAATCTTTGTCATACC | T/C |
| | 76695_628 | CTCTATAATTTCACTTCTGTAT | |
| | | GTCTGTTTACATGTCTATACAG | |
| | | TTATTTGCATGGCTGTCTCTTG | |
| | | AACATGGAAGAATGCCATCAAT | |
| | | GTTGGCATCTTTAATAGCAGGG | |
| | | GTCAGCAAACATTCTGTGCAAA | |
| | | GGACCAGGGAATAATATTTTAG | |
| | | GATTGTAGACCATATGGTATCT | |
| | | GTTGCAACTACTCAGTCTGCCA | |
| | | TTGTAGCAAATTGAAAGCAATC | |
| | | ATAGACAA | |
| | | ATGTAAATGGATGGGGATGGCT | |
| | | AAGTCCCTATAAAACTTTATTT | |
| | | ACAAAAACATACGGCAGGCAGG | |
| | | ATTTGGCCTATGGACCATAATT | |
| | | TGCTAACCTCTGTAACAGAGAA | |
| | | TCTATAATATCCTTAATATTCT | |
| | | GAAAAAAAAATCATTAATGAAT | |
| | | AAAAAAACATATGAATGAACTA | |
| | | CAACAAACAATTCCTTTTTTTT | |
| | | CCCCTCAACTTTAAGCCACAAA | |
| | | TTAATCCTGGAATAAGTAAGCA | |
| | | TTAGACAA | |

SEQ ID NO: 425

| | | | |
|---|---|---|---|
| 350589 | IBISS4snp140 | GCCTAAGTTTCCTTTTGACCAT | A/C |
| | | CACCTCCCAGTAACAATTTACT | |
| | | GTCAATTCCATTTCAGAATGGT | |
| | | CAAGGCTTCTGTTGAAAAGATA | |
| | | ACATGACCAAGA | |

SEQ ID NO: 426

| | | | |
|---|---|---|---|
| 350590 | IBISS4snp186 | TCTGCACTTCACTCGGTAATAT | C/T |
| | | TAGCAAATCTCCAAATGTTAGC | |
| | | CACATTGTTTGTTTCCCTTGTA | |
| | | TGTTGTTTATTCATGATACTTC | |
| | | AATGCTGTAACT | |

SEQ ID NO: 427

| | | | |
|---|---|---|---|
| 350636 | SCAFFOLD | ACAACAGCTTGAGAAAAGAACC | G/T |
| | 105892_4523 | TCTCAGTAGCTCGGAGGGAACT | |
| | | GCTCCAAAGAGGCAGGGGAAGA | |
| | | AGACAGTTTATATATGATTTTT | |
| | | TGCTGGGGAATACATGAAGTCA | |
| | | AGCACACATCTTGATAAAAGTC | |
| | | TGCTGGTTACAAGGAACAGATA | |
| | | TCTTGATTATTGATTTTAGTGC | |
| | | TTTTCTATGTAGGGGAAGATGC | |
| | | AAGAATTCAGGCCTATTAAAAT | |
| | | CTTTCCTGAGATATACATTTAA | |
| | | CTATCTAA | |
| | | GGGCCTGTTTTTCCAAAGCACA | |
| | | GAATGCCCTATTCTGTTTTCTG | |
| | | TCTTTAATTCCTTTCAGGGTGC | |
| | | TGTCCATCAGTGACTGCAGTAG | |
| | | CTAATGACTTGAACCTTGTACA | |
| | | ACTGGACAATGAGCAGCATTCT | |
| | | TTCTTTTACAACTTTTAAGTAG | |
| | | CCTCTGAAAATACTTGACATAT | |
| | | TTTTAAAACATAGTCAAGGCTA | |
| | | TAAAAATTTTTTAAGTAAATTT | |
| | | ACAAACTTAGTTTCCCTTTTAA | |
| | | GTATTTTA | |

SEQ ID NO: 428

| | | | |
|---|---|---|---|
| 350721 | SCAFFOLD | ACTTTATTAATAGACACACAGA | T/C |
| | 146466_8003 | CACTTGTGAGTATTGCAGATCC | |
| | | TGCTTTTCTAGATGTAGGATGG | |
| | | AGTTGAGGTTCTGCATTACCTG | |
| | | CAAGTTCCTAGATGAACCATGC | |
| | | TTCTGGTCCATGGGCCACTCTT | |
| | | TAAGAGTATAAAGAGATGGCTC | |
| | | CCAAACTTGGTTGCGTATTAGA | |
| | | GCTGCTTGGAGAGCTTTTAAAA | |
| | | ATCAGGATGCCAGGCCACACCC | |
| | | CTGACCAGTTAGATCAGAATCT | |
| | | CAAAGTGA | |
| | | GCCTAGGAATCAGTATTTTTAA | |
| | | ATTTTTTATAGCTGATAAGGGC | |
| | | TTCCTAGGTGTCGCTAGTGATC | |
| | | AGGAACCCACCTGCCAAGCAGG | |
| | | AGACATAAGAGACACTGTTGCC | |
| | | ATCTCCAGGGGTCAGGAAGATC | |
| | | CCCTGGAGGAGGTTATGGCCAC | |
| | | CCATTTCAGTATTCTTGCCTGG | |
| | | AGAATCCCATGAACAGAGGAGT | |
| | | CTGGTGGGCTACAGTCCGTGGG | |
| | | GGTCATACAGAGTCGGACACGA | |
| | | CTGAAGCG | |

SEQ ID NO: 429

| | | | |
|---|---|---|---|
| 350780 | SCAFFOLD | ACAATTTAGGAAAAATTGTAGA | A/G |
| | 166988_3684 | ATACCTAAAAGTATCAATCAGT | |
| | | AAAAGCCATTCATAACAGAAGC | |
| | | TATGAGATGAAGGCATGAAGTT | |
| | | TAACTGAAGACAGTAAAACAAA | |
| | | ACAAGCCAAGACAGAAGGGTAG | |
| | | ATTTAATAAAGATAAAAGCAGT | |
| | | ATGTAATCAACTATAAATAAGA | |
| | | AAAATGGCCGAACTAATAAATA | |
| | | AACCAAACACCAAATCTCTGAG | |
| | | CAAACCAATAAACCAGAAATGC | |
| | | AGAAGTCG | |
| | | AAGCACAAATTCATAAAATAAT | |
| | | AAATGATGGAGGAAAAATCATG | |
| | | ATATTTGGAGGAAATTAAAAGA | |
| | | TTGACAAGAGGCTATGCTGTTC | |
| | | AACTATGCAAAAGTAATTGAAG | |
| | | ACCCAGTGAAAAAAGGAATATT | |
| | | TTTAAAAATTTTCAATACATTA | |
| | | AATTAATTCCAACACAAACAGA | |
| | | ATATTTATAGACCATTCACTAT | |
| | | GGAAAAACTGAGTTGTGAAAAT | |
| | | GCTAATCCTATGTCAAAAATCT | |
| | | AACTCAGA | |

SEQ ID NO: 430

| | | | |
|---|---|---|---|
| 350915 | SCAFFOLD | CTAGAACCAGCTGTATGTGTAG | A/G |
| | 235117_21328 | GGGTGGTAGGGAGATGTTCAGA | |
| | | GAAACTTAAAAACCACTGGATT | |
| | | CTGTGCCAGGAATTTGCAATTG | |
| | | TAGTAGGTGTGAATTTACTGAT | |
| | | GACCCATTGCCTTTTTTTACAG | |
| | | CAGTGTTTCTCCATTTTTTAAC | |
| | | TCATTCACAATTGGTAATTAAA | |
| | | AATCTCATCCTCCAGTGTCAGT | |
| | | ATAACCCTTACTCCATCACCCA | |
| | | GACTGAAAACCACTAATAATCA | |
| | | TGAAACAT | |
| | | AGTCTCTCTAGCTCCATTATTA | |
| | | ACACCAATATAATATGAATTTT | |
| | | AAACATTTAACTCTTCATTTGC | |
| | | AGGCATCACAGACTCCACCTTT | |
| | | TACCTGGTTACCCAAAAGGCAA | |
| | | AAATACACCCATCTATGGACAG | |
| | | AGTGTTAAAGAAACACTTGTCT | |
| | | TTGAAAAATGGCCAATTAACCT | |
| | | TCTAGAGCAGAAATCCTTTCTT | |
| | | AGAAGTAAAATCATCTAAAAGG | |
| | | AAAAATTCATTCTATTTAAATC | |
| | | CAATTCTG | |

SEQ ID NO: 431

| | | | |
|---|---|---|---|
| 350996 | SCAFFOLD | ATGAAACTATTCTTCCCAGAAT | T/C |
| | 265171_9293 | GAGTAATATTGTTCCCTTCAAC | |
| | | TCCATGCCTTTGCAAAATTCTC | |
| | | TTATTTTTAAAGAATGCTCTTC | |
| | | CCTGTTTGATCTGCTTAGCCAA | |
| | | TACCTACTCATTTTTTAAGACC | |
| | | CTGTGTTATGCTTTCTTTGATC | |
| | | ACTCCCACCTTCCCCAAGCTTC | |
| | | CTTCCCATGAGTTGACTATGTT | |
| | | CTTGTGTCACTATCTCTAACAC | |
| | | ATATCCTTATCAAATACTGTAA | |
| | | TACGATTA | |
| | | GTACTGACATCTTGTCTCCCCC | |
| | | ACTGTGACACCGAGAAGGGCCT | |
| | | GGTGGGACTCCTGGGCACAAAG | |
| | | CCTTTCTGTGCCCATGACCTTC | |
| | | CCTGAGTTCCAAGGGGGAAGAT | |
| | | ACAACAGTTGTTAGTTAGAGAA | |
| | | AGGANNNNNNNNNNNNNNNNNN | |
| | | NNNNNNNNNNNNNNNNNNNNNN | |
| | | NNNNNNNNNNNNNNNNNNNNNN | |
| | | NNNNNNNNNNNNNNNNNNNNNN | |
| | | NNNNNNNNNNNNNNNNNNNNNN | |
| | | NNNNNNNN | |

SEQ ID NO: 432

| | | | |
|---|---|---|---|
| 351065 | SCAFFOLD | TCCTCTGCTTTCTGCTAAATTT | G/A |
| | 295013_6281 | CCTTGCTCACCAGCATGTACCA | |
| | | TTTCCCAAGGTCTGCTTCTTAG | |
| | | GACTTCTCAAATCCCTGGTGCT | |
| | | GCCCCAGAAATGTGACAGTATT | |
| | | TGACTTCTCCAGAATAGTAACC | |
| | | AATCTCTGCTGCACCCTTAGGT | |
| | | TTCCCAGACACTTCTATAATGT | |
| | | GTGCTTCATCCAAGTTTTCTGA | |
| | | CTATTGTATTCCAAACCTCCAT | |
| | | CATGTTGAAGTCACCACTTATA | |
| | | TCACTTCT | |
| | | TGATAATCATTGCAATATGTCT | |
| | | CAATTTCTCTTCAACATAGGAC | |
| | | CTGTAACTCAGCTGCTGGGAGT | |
| | | GTGGTTGGCAGACATTATTTAG | |
| | | TTGTCAGTCCCAATTCAGCTGC | |
| | | CTCTTGGGGTGGCACTCATGTC | |
| | | ATGATTTGTTGACAGGAGGGGT | |
| | | ATGAGGCCTAGCTCAGAACAAC | |
| | | CCTGAAAGTCAGCTCTAGTTCC | |
| | | AAAGTGTTCCATGGGGTAGGCT | |
| | | AAGTCTGTAATTGAACCTCAGT | |
| | | TACACTTG | |

SEQ ID NO: 433

| | | | |
|---|---|---|---|
| 351089 | SCAFFOLD | GAGAATGAAAGAGAAGTTGATG | G/C |
| | 303739_1450 | AATCATACCAGTTATAGAAAAT | |
| | | TGCTAATTTAATAAGCATCTCA | |
| | | TTAGAATTTGCTATGGTGACTC | |
| | | TGAAGTTTAATGAGCAGAAATT | |
| | | TTCCCTCTAATTTTTCTGAAGC | |
| | | AGTGAGATGCCAGTGAACTGAA | |
| | | TTGATGCTATAATAATAGTACT | |
| | | TTCTCCTCTACTTTCTTCTTCA | |
| | | TGAGAAATCAGCCCACATATCA | |
| | | TGCAATTCACACTCACATATAT | |
| | | CAGTTGAA | |
| | | GAGTGTGAGAAATCAGAAATCA | |
| | | TATAATTTACAGTGTTTAAATA | |
| | | CATTACACAGCAGGTACTTCAA | |
| | | TATCCTGGTTGTAGCCTTCTGG | |
| | | TAACTAGTTCATGTGTATACTA | |
| | | TGCACACTCATAGACAGTATGT | |
| | | TTGTCTAAGATAGTAAATTAAT | |
| | | AATAAAAGCAATAACTACTGCA | |
| | | AAGACCCTTAATACACACAGTA | |
| | | CATACTTTTAAAAATTCTCTTT | |
| | | AACATAATTCCTTGTACTATCA | |
| | | TCTAGAGG | |

SEQ ID NO: 434

| | | | |
|---|---|---|---|
| 351131 | SCAFFOLD | CAATACTTGTATATTAATGTCT | A/C |
| | 323835_5603 | GAGAAATACTGTTGAATTGTTT | |
| | | CCCCCAGATCTAAAAAAATTTC | |
| | | AATAAAATTCATAAACGTATAC | |
| | | ATAAACACTTACACACATAGAC | |
| | | ATTACCAAAACCACAAGAAAAA | |
| | | CTTTTATTAATCTAATCTTAGG | |
| | | TTTACTTTTAAAAAACCTACTA | |
| | | TAATTATTTTACCTTTTACCCA | |
| | | TTGTAGGAATAAAGTGCTATAT | |
| | | ATGCAAATAAAGAGATGTGTTG | |
| | | CATTGCTA | |
| | | GTTCTTGTAAAAGAAACTAAGA | |
| | | ATGTCATTGGTTTTACCACAAA | |
| | | TGGTAGTCCTTTCTAGTATTAC | |
| | | TCCTATAGAAAAACATACCACA | |
| | | TTTTTTAAAAATCCTACATTAT | |
| | | AGTATCATTATTTACATCTGCC | |
| | | ACAATTATATCTACCATTATTT | |
| | | AACTGATTTATATGTGAGACAC | |
| | | ATTCATTTATGCAAGTTTCCAT | |
| | | TAAAAGGTCAGGGATAGGGTCA | |
| | | AGAACTCTAATTCTGACTTAGG | |
| | | AACAATCC | |

SEQ ID NO: 435

| | | | |
|---|---|---|---|
| 351334 | IBISS4snp991 | AAAGATCACACACTGAGAATCT | C/T |
| | | CTACACAATCTAGGCACAGATA | |
| | | ATCTAAACTAAACTACTGTGAA | |
| | | ATTCTGCAGTACTGAAGTACTG | |
| | | TATGTTGCCCTG | |

SEQ ID NO: 436

| | | | |
|---|---|---|---|
| 351593 | SCAFFOLD | GCAGCTTGGGCCCGGCATGGAG | C/T |
| | 234818_2292 | TGGTTCTCCTGGGACACCGGGA | |
| | | CTGGCTGGATTCTCTCCTAACT | |
| | | CAGCAGAAGTGCCAGGGCCATG | |
| | | CCAGTCACAGGGTAAAGTGGCT | |
| | | GGGGTGTGAGCCCTACACAGAG | |
| | | AGGGTCTGTAAAACCCTTCTTA | |
| | | CTGACTCAAGCCCTGACTCAGG | |
| | | CATTCTAGGATTTCAGGACTCA | |
| | | CCCAAACCTGATTAAAGGTGTC | |
| | | CAGAGGGCCTGGGGTTAACACT | |
| | | GAGTTGTT | |
| | | TTTAAGTACAAGATTATGGTTT | |
| | | CCAATAAAGTGGATTTGTGGTA | |
| | | CCTGAGGTGATTTTGTGGAAGA | |
| | | AAAAAAGGAAGATTAAAAAGTC | |
| | | TGTGTCAAAGTAGTATTTTGAT | |
| | | TAGACCAGAAGCATAATTTTAG | |
| | | AAAATGGAAAATAGTAGGGATG | |
| | | CCATAGGAAATGTGCTTGGGGC | |
| | | CTGCCCTCCCCAGAGGAATGGA | |
| | | GTCAATAAATCATCTCCCGCCA | |
| | | GTCCCAAGTTTTCAAGTGTGAT | |
| | | GTTTTCGA | |

SEQ ID NO: 437

| | | | |
|---|---|---|---|
| 351654 | SCAFFOLD | AGGGGCTGGCACAGCCTCTGAG | T/C |
| | 26216_19609 | GCTGTTGCAAAGTCTCAGCTAC | |
| | | CACCACCACCACCCCCGCCTTG | |
| | | TCAGTCTTATATAACACCGTCC | |
| | | ATGCCACACACCGCCACCTTGT | |
| | | CCCGCTGAGTCAGTGCTCCTGT | |
| | | GCTGCCCCCCAGGTCCTGTGTG | |
| | | GCAAGAATCCCCTGAAGCCCAG | |
| | | CGGCCCCCTGGGAAGGTGCGCA | |
| | | GAGCAGGCTCCGGGTTACAGAT | |
| | | GCAACTTCCCTCCCAGAAATGT | |
| | | TCCCTACT | |
| | | TAATCACTTTTTTAAACTTCAC | |
| | | AAACACAATCTCTGCACCGTGA | |
| | | TCAATTATTTAGCAGAAAATGA | |
| | | TGCATCGTGTTAGCAACTAGAT | |
| | | GGCATTTAATATGCCCCCAGAA | |
| | | TTCTAATGACATGCCGTCTTCA | |
| | | GAGGCCTAATGCAAAGTTTCCC | |
| | | CTGCCCATGTTTTTGAAGACTT | |
| | | CCCAGAGTGGAAAAGAGCCAAG | |
| | | ATCTATATGCTCTCAGTTGATG | |
| | | TCGGATGTAGGTGGCCCCTGGA | |
| | | ATGTAATC | |

SEQ ID NO: 438

| | | | |
|---|---|---|---|
| 351663 | SCAFFOLD | CTGTGTTAAAACTTACATATAG | C/T |
| | 266133_5910 | TAGGCATTCAATGTTTTTATTA | |
| | | CTATCATTATTTTACATGTCAA | |
| | | TAATGATATAATAAAATGTAAT | |
| | | TTTATTACTATATAATAATGAA | |
| | | TGAGATAACATAACAGTAATAA | |
| | | AACCTGTTTCTATTTCAAATAT | |
| | | TTTTATCAACATACAATTAAGG | |
| | | GAACCCTTGTCAATTAACTCAT | |
| | | TAACAGATATTAAGTAACTATA | |
| | | TGTTATAGGCAGTGTTTAAGTC | |
| | | TTGAGTTA | |
| | | TCAGTAGAACTGATCTTTAATA | |
| | | TCATGGGTATTTCACACTGATA | |
| | | AAAGTAACCATACATAAATAGC | |
| | | AATAAAACAAATAAGCAAAAAA | |
| | | TAGATAAGAAAAATATTGGAAA | |
| | | ACCATAAGAGTGCTTAGAAATA | |
| | | TACTTTACACAGAAGTGACAAT | |
| | | GTGAACTAATATCTGCATGTGG | |
| | | AAAAAGATCAAGTTAGGTAAAT | |
| | | CTTTTGACACTGGATAAATAGT | |
| | | TGACTGAAAACACTAATATATT | |
| | | TCTCAACTT | |

SEQ ID NO: 439

| | | | |
|---|---|---|---|
| 351687 | SCAFFOLD | TCAATTATTGTCTTGTAATAGA | G/A |
| | 277056_3839 | CAAAGAATATTACTGGTTGATT | |
| | | CTTAAAGATAACTGTCCACTGA | |
| | | ACTGAACTGTACTATGATTCAA | |
| | | ATGATTTTTTAAAACAATGTAT | |
| | | TTATAAAGAAATATATTCTGAA | |
| | | CTATGTACAAAGTTATTTTTGT | |
| | | GTACTATTATGATATGTATATA | |
| | | TTTTCAGTTCCATGTGTTTACA | |
| | | GTTTAATACATCAAGGAACAAC | |
| | | CATAATGATAATAGTTTATATT | |
| | | TAGCAAGT | |
| | | TTTACTATGGGCCACACACAGT | |
| | | TCTTAATGATTTCCGTATATTG | |
| | | ACTGAATTCCTACAATATTCCT | |
| | | ATGAGATATACTATTATTATTC | |
| | | TCATTTAATATATCAAGGAAAT | |
| | | GGACCACAGTGAATTTTAAGTA | |
| | | ATTTGCTCAAGCTAATGAGTGG | |
| | | TGGAATCAGAGTGTGAAATCAG | |
| | | GCATATAGTTCCAGAGTTTTTG | |
| | | TTTTTCAGTTCTTTTCAGTTCA | |
| | | GTCGCTCAGTCATGTCCAACTC | |
| | | TTTGCGAC | |

SEQ ID NO: 440

| | | | |
|---|---|---|---|
| 351724 | SCAFFOLD | GTAGTAATCTCTGCTCACTTGT | A/G |
| | 295446_12626 | TAGATATTACCAATGAGATAAA | |
| | | TACAGTGGATTGCACTTATCAA | |
| | | TTATTCAGCAGTGTGTAAATTC | |
| | | AGCTACTTAGAAAGTTGTCAAA | |
| | | ATATTCTCAGAATAGCAGCAAT | |
| | | TCTTAAATGAACTATAGATACA | |
| | | TAGATCTTAGAACATGTACCAT | |
| | | GATTAGCCAGGATAGCAGGCAA | |
| | | AAAGTAGAGTTTCTTTGTTTTC | |
| | | TTCTTTTTTTTTTTTCTTTGTT | |
| | | TTCTGACA | |
| | | TAGCTCTCGAAGTCAATTTTAG | |
| | | CTGGCTCTAAGGTCTAGTTTGG | |
| | | AGAGAGAAAGAAGAAAAACTAA | |
| | | GAAAAGAGCAGAAGCTGGCATA | |
| | | AATGTAACAATTAGAAGTAACT | |
| | | ATAAATAGTGGAAAGCTATTCA | |
| | | CTGAAAGCAGCAGTAAATGACA | |
| | | GAGAACAGGAACAGAAGTGAGG | |
| | | GAGAAGAGAAGAAAGAACACTG | |
| | | AGGCAACTGATCTTTTCAGTTT | |
| | | AATAGCCTAAAAAGTACCCTAG | |
| | | TATTTGAG | |

SEQ ID NO: 441

| | | | |
|---|---|---|---|
| 351743 | SCAFFOLD | TTATGACTTCATGGACTATAGA | A/C |
| | 301712_12074 | GTCCATGGAATTCTCCAGGCCA | |
| | | GAATACTGGAGTGGGTAGCCTA | |
| | | TCTTTTCTCCAATGGATCTTCC | |
| | | TGACCCAGGAACTGAACCAGAG | |
| | | TCTCCTGCATTGCAGGCAGATT | |
| | | CTTTACCACCTGAGCTATCAGG | |
| | | GAAGAATTCAATATCGGCATTT | |
| | | TTTTTTATATCAGCACTTTGAA | |
| | | ACTGGAATTTCAGTGTCTTTTG | |
| | | ACTTTCATCATTTTAGATGAGA | |
| | | TTAGGGCT | |
| | | AGTTTTATTGTAGTTCCTTTGA | |
| | | CAGGAATATATTTCTTTTTCTC | |
| | | TGATACATACTTTTTCCTCTGT | |
| | | GAATTTTAGCAGTTTTACTGTC | |
| | | ATGTGCCTAGAGGTAGTTTCTT | |
| | | TGTATTTATTCTGTTTTGGATT | |
| | | CATAATTTACTTTGAATATTAG | |
| | | GATAATCTCCTTCATTAATTTT | |
| | | GAAAAATGATTGGCTGCTCTTT | |
| | | TCAAATATTGTTCCGTCTCCTT | |
| | | TTCTCTCTCCTTTCCAAGGGGA | |
| | | ACCCTATT | |

SEQ ID NO: 442

| | | | |
|---|---|---|---|
| 351863 | SCAFFOLD | TAATTATATGTTATTTGTTGAT | A/G |
| | 59158_530 | AGCTTCTCATAAAGAAATAATT | |
| | | TTAAAGAAATGATAACCCTCTG | |
| | | TCAGGATCTAAAAGTGATTTCA | |
| | | GGATTCCAGTGCCTCTGAACTG | |
| | | ACAGCAATTGTTGCACAAGGTG | |
| | | ATTGAGGTGGAAAATGGAAGAC | |
| | | AAGAGCTCCATCTTCTCTGTCT | |
| | | CTGATACATCTTAGAAGCCTAT | |
| | | TTCTTCTTAACTAAATATATGA | |
| | | TCTTTTTGTTATGTTTACCTTT | |
| | | AGCCTACA | |
| | | TGTAGCTTGTTTTACAGAAAAA | |
| | | GTATCTCAAATAGCATCAACTC | |
| | | TATTTTTGGAAACATAAGCATT | |
| | | CTTTTAAATTTAAGTAGTCTTT | |
| | | TTCAAGGTTACTTTTTTCCCAT | |
| | | CACAGATCATTTCCAACCTTCC | |
| | | CATTGTTTTTTCATTATCAACA | |
| | | TATATTCTGTTTTTCTGAACCT | |
| | | ACACAAGCTTTATCATTGCATG | |
| | | TTTTAAGAAGCATTTTCTTAAA | |
| | | GTCCTTAGATTATTCTGTCTAT | |
| | | CCATTTCT | |

SEQ ID NO: 443

| | | | |
|---|---|---|---|
| 351993 | SCAFFOLD | GCCCCTTTGTGTAACAAGCTTT | T/C |
| | 114762_1553 | CAAAAGAGGCAAAACGTGGGAC | |
| | | TTCAGGATTCACTAACCGATCA | |
| | | TAAGTGTGACCCCAAGGGTTGT | |
| | | GTGACCAGCAGAACCCAATGAA | |
| | | GCTTCAACCTTAGAATATAGCC | |
| | | ATCCCATGAGGTTCATGTTTAC | |
| | | TGTGCTTCCCTGGAAGAGAAAG | |
| | | AAAAAAAATCATCATAATTTTC | |
| | | ATTTGTATCTGTCCTGCTTGAA | |
| | | AAAAAAAAAGATGTGAGTAGCT | |
| | | TAGAGAAA | |
| | | AGGTATAATAGGGATCATTAAA | |
| | | AAACAGATAATTATATAAAAAA | |
| | | AAAACACTACAAGGAAAAATAA | |
| | | CAACCTAAGATAAAGCCACAGC | |
| | | TAAGATTACTACACAAAAATGG | |
| | | CAACTTATAGAAAATGCTAGTA | |
| | | TTCAAAACAGCACAGTACATCT | |
| | | AGAACTTCCTAGTAGCCCAAAC | |
| | | AAAAATAAAACACAAGTTACCA | |
| | | AATTTACAGGGTACCCAAGACA | |
| | | ATAATAAACTTTGGCTCAGAAA | |
| | | AAGCACAG | |

SEQ ID NO: 444

| | | | |
|---|---|---|---|
| 352138 | SCAFFOLD | GTTTGCAGAATCTTATTTTATA | C/G |
| | 190076_2021 | AAAGCAATTCAAGTCAGTGGAA | |
| | | CTGAACAAGGAAAAAAGAATGG | |
| | | AGAACAAAGAATTCCATTCACA | |
| | | TATTTATAAAACTAGAGCTATA | |
| | | TCTGTTATATCTCTGAAAGGCA | |
| | | GAATGATATAGCAATTATTACA | |
| | | AACACAGTTGTTTGAATATATA | |
| | | CTGACAGACATTCCACTCCTCA | |
| | | AAATTAAAGAATCATTTTATCT | |
| | | TCAGGGTTTCAATTTTAATTTG | |
| | | AGCTAAAT | |
| | | CAAATCGAACTAACCATGGATG | |
| | | TTTATTCCCCTTCTTTTTGGAC | |
| | | CAGAAAACAAGAAACAAAAATA | |
| | | TGTGTTTTTCGACTGATCCTCA | |
| | | CTTCTCCACATGAGGATTCTGC | |
| | | CTGCAGACCCTGTTGCAGAAGC | |
| | | TTGGATACACATGGTGATGAAA | |
| | | GGTCATTTTACAACTTCACATG | |
| | | TTCCAGATGGAGACTCTTGAGA | |
| | | AAGAATCTCTTTTCTGGTAAAA | |
| | | GCCAGAAAAAGAAACTTCACAC | |
| | | CATTTAAC | |

SEQ ID NO: 445

| | | | |
|---|---|---|---|
| 352221 | SCAFFOLD | CATTTGATTTCTGCATTGTATA | A/T |
| | 236442_327 | CAGTGTGTGAAAACTATGTGAT | |
| | | ATATAGTGAACTTCATTATTAA | |
| | | TTTCCTTGTCCATAGTTACAGG | |
| | | TTAAAACAGCCTTGTGTTGACA | |
| | | ACATAACAACACCAAAATTAAA | |
| | | AGACTATTTCATCATATTTATA | |
| | | CTTTTACATTAGTACAGATTTT | |
| | | GGAAGATGAGTTACTGTTGAAT | |
| | | AACAAATCAGAGCCTCGCTCTC | |
| | | AATTTTTCCTCACGATAGTGAT | |
| | | CCATTTCT | |
| | | TTTAAAACTCTCAATTTCATAT | |
| | | GATTTAATTTAACTTGATTATA | |
| | | AAGGTTAAGTTTAGGGTTATAA | |
| | | AAAGTCATTTTCTAGACTTAAC | |
| | | TGAATTTAGTTTGAAAATCCCA | |
| | | GAATTCGATTTATTCAATGTGA | |
| | | ATAAAATATTCACATATGATTT | |
| | | CAGAGCATTATTATAATTCCAA | |
| | | TTTTAATTTTAACTTCTTTGTT | |
| | | AAGGTACTAGCTCCAATGTAGT | |
| | | AAAATGCATTTGATAGTAACAC | |
| | | TCAAAATA | |

SEQ ID NO: 446

| | | | |
|---|---|---|---|
| 352556 | SCAFFOLD | CATGAGCATAATTGTTGGATGC | C/A |
| | 125013_22381 | TATTTTATTACATATGATCATA | |
| | | CCTGAATTTTATTTGTGTCAAT | |
| | | CAAGTGCTGTGCCATGGATTTT | |
| | | AGGATAATTGCAAAGAAGAACC | |
| | | AGGAATGCTGTTAGGAAAAGAA | |
| | | AAGACAATCAGAAATTTATTAT | |
| | | CTTACAGCAAAGTAATGATAAT | |
| | | TCTGTTCTTTACAACATGCGAT | |
| | | ACTATAAAATCTTTATCTTGGA | |
| | | ATGTATTCTTTGAAATTAAATG | |
| | | CATCAGAG | |
| | | AATAATATAGTCTCCATGATGT | |
| | | TAGGATCATCCTTAGCAAAATT | |
| | | CTAATGGGAAATGTAAATACCA | |
| | | GATATTTCTGTTTCCCAGTCAT | |
| | | CTCTAAGACTGTATAACACAAG | |
| | | AAACTGGCTTATTTGACATGGC | |
| | | TGATTAGACAGTCGTCTTAGGA | |
| | | AATACTTATTCATGGGGTGTTC | |
| | | TACCTGTTTTCCTTAAACAAAA | |
| | | CTAAATGTTGTAGCAGGCCCAC | |
| | | CCCTCACATTACGATGGGTAGA | |
| | | GGCGAGTA | |

SEQ ID NO: 447

| | | | |
|---|---|---|---|
| 352701 | SCAFFOLD | CCTTTCCAAGTACTGGGGAATG | C/A |
| | 205002_91700 | TTTAAGAGCACACATAAACACA | |
| | | TAGCCATCAAACTGATTTGAAT | |
| | | TGGACAAAATAAACAAAATCTG | |
| | | GTCTAAATGGGAGTTTGTTCAG | |
| | | TTATGTCTCTCAGTTTTCAAAA | |
| | | GCCTTGAAAGAAAAATTCACAG | |
| | | CTTGAATGTCTCAAAAGAATCT | |
| | | TCCAGGGGGTTAACAGATTTGT | |
| | | TTATTAATAAAAGCTAAATCAG | |
| | | AAGGAACTTAAATTACCTCTGC | |
| | | TTTCACA | |
| | | TATTTTAAAGAGGAGATTTTCA | |
| | | TACTGGTTAGGGACTGCCCTAA | |
| | | ACAATTCTCTGAAATGGGCAAT | |
| | | ATCAGTTTGTAAATGTAAAGAT | |
| | | TCATTGGCCACTGAAACTCAGA | |
| | | GACTGTCAAATAGCTTCCATTC | |
| | | TCTCATTTCACAGACAGGAAGC | |
| | | CAACACAAAGGAGAAAAGAAGC | |
| | | AACCTGCTCCAAGTGTATAACT | |
| | | TGGTAATAGAATTCAGATTAGA | |
| | | ACCCATGCCTTTGAGAGCAAAA | |
| | | GTTTTTTT | |

SEQ ID NO: 448

| | | | |
|---|---|---|---|
| 352760 | SCAFFOLD | ATACTTTCAGAGCCTCCATCTC | A/G |
| | 245260_13707 | AGGGCCTCGGATAGTAGCATCA | |
| | | GATGTTTGTAGCAGGAAGTCAA | |
| | | ATTTAATCGGTTTCTCTGTGAC | |
| | | AGATTTAAAGTCCATCAGGTCC | |
| | | TGGGGAAAGATCATCTGAAGAG | |
| | | CCAGAAGATGAGCTCATTTGCC | |
| | | TCTCCTCAAAGACAAGTTAGTG | |
| | | AAAAAATTCAACAGAGGAGTGT | |
| | | TTAGCAGGTGGAAGCTCATAAA | |
| | | TCTGTCAGCAAACTTTACTGAC | |
| | | CATATACA | |
| | | GAAATTAGGTTATGCTTTTTTT | |
| | | TCCCTTACCTCCAGTGTCATTT | |
| | | TGAATTATGTACTCTGCATTAT | |
| | | GGAGAGACCAGCTTCTTCCTGG | |
| | | CTGCTAGCCAGGCATTTGGAAG | |
| | | ACAGGAATTGGTTTTATTTTTC | |
| | | TATCAACCTGTTTCTGATTCAG | |
| | | ATTTTAGGGTGACATATAGTCA | |
| | | GTATCAGGTTAAAAAAATAATC | |
| | | TGTTGTAGGTCAACCAACCCAC | |
| | | TTTCAATTATACTCAACGGCAT | |
| | | ATAATTGG | |

SEQ ID NO: 449

| | | | |
|---|---|---|---|
| 352783 | SCAFFOLD | TGTAAGCTTCTGTTTGGTGCCT | T/C |
| | 259826_8971 | CTCTTTCTTGACTTCTAATAGC | |
| | | CTACCCCTGAAGCTCTTTTAGG | |
| | | AGTGTTTATTCAGAATCAAGAC | |
| | | CATGCAATTGAGTTCTGACCAA | |
| | | ATCAGTGTAGGTGGAAGTGATG | |
| | | TAAATGGTTTTGAGAGCTGGTT | |
| | | ATTTAAAAATAGTTAAATGTTG | |
| | | CCTTTTGGTAATGCATGATAGA | |
| | | AGGAGCTCAGAATTCTGAGTCA | |
| | | CTGAGAAAACTATCCCCAATTT | |
| | | ATAAACAA | |
| | | CTCTGTAACCCAGAAATAATTC | |
| | | TCTATTTATTGAAATCACTGAG | |
| | | ATTTTAAAGTTTTTTTTTGGTT | |
| | | ACTTAATGGACTGGTTAGTCTA | |
| | | GTCCATTTTGACTAACACAGCA | |
| | | TCCAAACTTCGCTAATGTACAA | |
| | | TTTGTCAATCAACAGAAGACTT | |
| | | AAATAAATATTTGTTGCATAAT | |
| | | AAACATTATTTGCATTATAAAA | |
| | | TTCATAAAATATAAAAGACAAA | |
| | | TTAAGAGTTTAAAAAAATAGAC | |
| | | TTTCCTCC | |

SEQ ID NO: 450

| | | | |
|---|---|---|---|
| 352915 | SCAFFOLD | AGTAGATACACAAGTCACTGGT | T/G |
| | 40648_11101 | ACACAACACATGTTCTTACTGA | |
| | | TAAGTAGCATATATTGGCTTAA | |
| | | AGTAGGAAGACCTCAGTAAGAA | |
| | | ATTAAAGAATAAGTGACAAAGC | |
| | | AAAGCCATGACATGAGGGAAAA | |
| | | TATTGTCTATTTCTAACACAAG | |
| | | AACAACAGTTCCAGTCAAAAAG | |
| | | TCTAGAGTCAAATAAACGTGCA | |
| | | ATCACACAAACACTGTAATTCT | |
| | | TTCATGTTTCAATATCTACTTT | |
| | | TTTATAAT | |
| | | AATCGGTTACCAAGAAGTCCTT | |
| | | AACTGAATGATGAAACCAACTT | |
| | | ANNNNNNNNNNNNNNNNNNNNN | |
| | | NNNNNNNNNNNNNNNNNNNNNN | |
| | | NNNNNNNCAATTTTATGAAGTA | |
| | | TTGTTATTACTTCTGTATACTT | |
| | | GCACTAAGAACTTTAAAATAAG | |
| | | CCTAATCCACTGAAAAATTCAA | |
| | | TATATAATTATTAAACAGTGCC | |
| | | TTGAAGAATATATTCCCACTGT | |
| | | AGAAAAGGATTACTAACAGAAC | |
| | | TTGTGGGA | |

SEQ ID NO: 451

| | | | |
|---|---|---|---|
| 352985 | SCAFFOLD | GGAACGACTTGGGCTCTATGGT | A/G |
| | 85236_15670 | GACCTAAACGGGATGGAATCCC | |
| | | CAAAAGAAGGGATGTATGTACA | |
| | | TGTATGGCTGATTCACTTTGCT | |
| | | GTACAGCAGAAACTGACACAGT | |
| | | GGTATACAGCAACTGTGGTCCA | |
| | | ATTAAAAAAAATAAAGGCAAAT | |
| | | TGATGGAGAACACAAAGCATTT | |
| | | ATATTATTTTTCAAAAATAATG | |
| | | ATTTTGTCACTAAAATGCCCTC | |
| | | TTATACCAAATACCATGTATTT | |
| | | ATTTGAGT | |
| | | GGTCTTATATAGGTTCTTCACC | |
| | | CAAACACATGTATACCTGTGGC | |
| | | GGATTCATTTTGATATTTGGCA | |
| | | AAACCAATACAATATTGTAAAG | |
| | | TTTAAAAACAAAAACAAACAAA | |
| | | AAAACTTGTGTAAACCAGGCCC | |
| | | ATTTCAACACAGAGTCGAGAAA | |
| | | GTACATTTCACCTCTAAGTTTT | |
| | | AAAGGTGATGTGGTGTTTGTTA | |
| | | CTGTTACGTAGCACAGAGAAAA | |
| | | TATTTAAGCAATCACAATAAAT | |
| | | GAATTTCGT | |

SEQ ID NO: 452

| | | | |
|---|---|---|---|
| 353039 | SCAFFOLD | GGTGACTCAGTAAAGAATTCAC | C/A |
| | 109850_10815 | CTGCAGTGTAGGAGACACAGGT | |
| | | TCAATCCCTGCATCAGGAAGAT | |
| | | CCTCTGAAGAAGGAAATGGCAA | |
| | | GCCACTCCAAGATTCTTGCTTG | |
| | | GAAAATCCCCTGGACAGAGGAA | |
| | | CCTAGTGGGCTACAGTCCACAG | |
| | | GCTCACAAAGAGTTGGACGTAC | |
| | | TGAGCACACATGCACAGGGACG | |
| | | GTGTATTCAGTAAATAACTTCA | |
| | | ATTTAATGAGATTTCAATGTGC | |
| | | CTTTATAA | |
| | | TAACAACTATTTTTGAGTGATT | |
| | | TTTTTTAAAGGCATTTTCAAGT | |
| | | TTAAACTGCAGTGAACCACTAA | |
| | | TCTGCTTTCTGTTTCTATAGAT | |
| | | TTGCCTGTGCCTGACATTTTAA | |
| | | ATAATGGAATCATACACTGTGT | |
| | | GACTTTGAGGTCTGTTTTTTTT | |
| | | TTTTCCACTCAGCATGATGTTT | |
| | | TTGAGGTTCATCCACTTGTAGC | |
| | | ATTGTCAGAACTTCATTCCTTT | |
| | | TTATGGTCAAACAATATTTCTT | |
| | | TATGGACA | |

SEQ ID NO: 453

| | | | |
|---|---|---|---|
| 353054 | SCAFFOLD | GCTGGGGCAGGAGCTGGCCCCC | A/G |
| | 121064_3755 | TCTCTTCCTCTCTCAGATGCCT | |
| | | CTGAATTCTGAATTTACCGTGT | |
| | | CAAGTAGCTCTGTTCTAAAATC | |
| | | CTCTACATCCTTCCTGGGTTGT | |
| | | TATAGAGAATCTGTCCTTGAAA | |
| | | ATTATGCTCAACAAGCTAATGC | |
| | | TGTTTCAGTTCGGATTTTATTC | |
| | | ATGTTCTGCTTATTATAAGTGA | |
| | | CAGGAAACCTAGTCGAAAACTT | |
| | | CTTAAGTGAAAATGAAAATTTT | |
| | | GCTCCTAA | |
| | | ACTAACACAATATTGTAAATCA | |
| | | ACTACACTTCATTTTTTTTTTT | |
| | | AAAGGAAAACTTCTTGTCCCTT | |
| | | TAGTGTAAAAACGCAGGGACTT | |
| | | CCCTGGTGGTCCAGTGATTAAG | |
| | | CCTCCGTGCTTCCCCTTCCAGT | |
| | | GCAGGGGGCAAGGCTTTGCTAC | |
| | | CTGGTTGGGGAACTAAGATCCC | |
| | | ACATGTCATGCAAGCAAAAATA | |
| | | TAAAAGTAAGATTAAAATAAAA | |
| | | TGCAGGGATTTCAGCCATGGCT | |
| | | GAATCCAG | |

SEQ ID NO: 454

| | | | |
|---|---|---|---|
| 353060 | SCAFFOLD | AAGACTCTGGCAGGCTACACTC | T/C |
| | 125013_22651 | CATGGGGTCAAAAAGAGCTGGA | |
| | | CATGACTGACTGACTAACACTA | |
| | | TCTCTGGGGGAGGCTAAGATCT | |
| | | CACATGCCTTGCTAGCAAAAAA | |
| | | CCAAAACATAAAAGATAAGTAA | |
| | | TATTGTAACAAATTCCATAAAG | |
| | | ACTTTAAAAATGGTCCAGATAA | |
| | | AAAAATTCTTAAAGCAGTGAGC | |
| | | ATAAATCAGTAAGGTTTAGAAA | |
| | | ACAAATAAAAATGGAACTAATA | |
| | | AGAGCATA | |
| | | TCATTATTCAGAGAATATTCAT | |
| | | GAGCATAATTGTTGGATGCTAT | |
| | | TTTATTACATATGATCATACCT | |
| | | GAATTTTATTTGTGTCAATCAA | |
| | | GTGCTGTGCCATGGATTTTAGG | |
| | | ATAATTGCAAAGAAGAACCAGG | |
| | | AATGCTGTTAGGAAAAGAAAAG | |
| | | ACAATCAGAAATTTATTATCTT | |
| | | ACAGCAAAGTAATGATAATTCT | |
| | | GTTCTTTACAACATGCGATACT | |
| | | ATAAAATCTTTATCTTGGAATG | |
| | | TATTCTTT | |

SEQ ID NO: 455

| | | | |
|---|---|---|---|
| 353318 | SCAFFOLD | TAAAACTAATCAGAACAGATTT | A/C |
| | 316764_15803 | CCTGGAGCATGAAACCCTAACT | |
| | | AGGTTCTCTACTAAAATGTACA | |
| | | CTAAAGTCAGCTCCAAAGTATG | |
| | | ACTCAACTCAGTGGAGTATTTG | |
| | | CCTAAGCATTCCAGAATAGCCA | |
| | | GGGTAGGTGATGCACACAGTTT | |
| | | GTTGACGTGAGCAAGGAAACAT | |
| | | GAAAGAGATCTGCTTAGAGATC | |
| | | TGGGAACTATGTTTCATGGTTT | |
| | | GCTAATATGAGTTATTATTAGC | |
| | | AATCAGCA | |
| | | TTCTTAAAGATCAAAAAAAAGT | |
| | | ATACAAAGAAGAGAGAGGAAAG | |
| | | TGTGTTTAGCTTTTAAATATGT | |
| | | ATCATAAACCACAACTTAGATC | |
| | | TGTTTTCTGGTGATTTGGCTCC | |
| | | ATTATTTTTCCAGATGTCATTA | |
| | | TTCCTATTTTTTAACACTACCC | |
| | | ATCTGCAGAAGGGCTCAAATAT | |
| | | TCAAACCCCACCACATAGCTTT | |
| | | TCCTGTCTATATATCTAGTCTG | |
| | | ACTTCATCAAACACATACACAT | |
| | | TTTTTTTT | |

SEQ ID NO: 456

| | | | |
|---|---|---|---|
| 353634 | SCAFFOLD | CTCTTTAAAATCTAACCTTTCT | T/C |
| | 306770_1012 | TCTTCCTGTCTTCCACTATGAT | |
| | | TTCCCATACTGGAAAGCTCAGT | |
| | | TTTAGTGAAGGTCTATGTTAGC | |
| | | CCCTTGTTTCTCATTAACTTAG | |
| | | ACCATGACAGTGAATGTCCTGT | |
| | | CACTGATGGCAGAGAAAATCAA | |
| | | GAAGATGAGTAATTAGATGGTC | |
| | | TGGAAGATTTCCTATCTCAAAT | |
| | | TTACTTCATGCTGTCCCTGTTG | |
| | | TTTGTTTTTCTTCCTTTGATTA | |
| | | AGTACCAA | |
| | | CAAAATGAAATTAAAATTAAAA | |
| | | GATGCTTACTCCTTGGAAGAAA | |
| | | AGTTATGACCAATCTAGATAGC | |
| | | ATATTGAAAAGCAGAGACATTA | |
| | | CTTTGTCGACAAAGGTCCGTCT | |
| | | AGTCAAGGCTATGGTTTTTCCA | |
| | | GTGGTCATGTATGGATGTGAGA | |
| | | GTTGGACTGTGAAGAAAGCTGA | |
| | | GCGCTGAAGAATTGATGCTTTT | |
| | | GAACTGTGGTGTTGGAGAAGAC | |
| | | TGTTTAGAGTCCCTTGGACTGC | |
| | | AAGGAGAT | |

SEQ ID NO: 457

| | | | |
|---|---|---|---|
| 353643 | SCAFFOLD | CTAGAGGGAAGAAGACAGCTTT | G/C |
| | 316920_18038 | GAGAACTTTTGGGAGGCAAATA | |
| | | GGACTTGGTGATTGATGGCATG | |
| | | TGCCAAGTCAGGGAGTTAGAAG | |
| | | ACTCCAAGATGAATCTTGGATT | |
| | | TCTAATGTGGACCAATGGACAT | |
| | | TTAATGAGAAACAAAACTTAGA | |
| | | AGAGGCAAATTTGGGGGAAACA | |
| | | ATAGTGATGACAAGTTAAACTT | |
| | | TGGCCTTAATGAGTTTGATGTG | |
| | | CCTGTGAAGGCTTATAATTATA | |
| | | TAATACTG | |
| | | ATTTCAGTACAATAGCAACTTT | |
| | | TTTTTTTGGCTGCATCAGGTCT | |
| | | TCACTGCATTATGGGGAGTCTT | |
| | | TCATTGTTGTGCAGATTCTCTA | |
| | | GTTGTGGTGTGTGGGCTCCGGA | |
| | | GTTGCAGCATACAGGCTTAGTC | |
| | | ACTCCAAGGTATGTGGGATCTT | |
| | | CGTTCTCCCCTGCATTGCAAGG | |
| | | CAGATTTTTAACCACTGGACCA | |
| | | TCAGGGAAGTCCCACAATGGCA | |
| | | GCTTTGAAACCTCTGTCTGTGT | |
| | | GATGTTGA | |

SEQ ID NO: 458

| | | | |
|---|---|---|---|
| 353681 | SCAFFOLD | NNNNNNNNNNNNNNNNNNNNNN | T/C |
| | 60886_7763 | NNNNNNNNTCATTTCCGACTCT | |
| | | TTGCAATCTCATGGACTGCAGT | |
| | | ACACCAAGCCTCCCTGTCCTTC | |
| | | ACCATCTCCCAGAGCTTGCTCA | |
| | | AACTCATCTACTGAGTCAGTGA | |
| | | TGCCATCCAACCATCTCATCCT | |
| | | CTGTCGCCCCCTTTTCCTCTTG | |
| | | CCCTCAATCTTTCCTATCATCA | |
| | | GGGTCTTTTTCAGTGAGTCAGC | |
| | | TCTTGCACAATAAAAAAACTTT | |
| | | ATAATTCA | |
| | | GCCATTTAAATCCCAAGATAAC | |
| | | TTTATATGGTTAGCATTTTTAA | |
| | | AAAACATCTCATTGCTTCTTAT | |
| | | GCAAACGAAAGTAAAAAATGAA | |
| | | GTCTCTCAGAAACTGATATTAA | |
| | | GCAGACCAAAACCAACTCTTAA | |
| | | GAATGTAGATTTAAACATCATA | |
| | | ATTTAAAAATCTGATCGAGCAG | |
| | | ACAGAGAATTCTCTACCCCAAA | |
| | | ATAATGTCTTAAATTTATTTAG | |
| | | AACATCTAAAAAATGATTGGCT | |
| | | TTGCTGGT | |

SEQ ID NO: 459

| | | | |
|---|---|---|---|
| 353752 | SCAFFOLD | TAGAAAACAAATAAAAATGGAA | C/T |
| | 125013_22437 | CTAATAAGAGCATATTCATTAT | |
| | | TCAGAGAATATTCATGAGCATA | |
| | | ATTGTTGGATGCTATTTTATTA | |
| | | CATATGATCATACCTGAATTTT | |
| | | ATTTGTGTCAATCAAGTGCTGT | |
| | | GCCATGGATTTTAGGATAATTG | |
| | | CAAAGAAGAACCAGGAATGCTG | |
| | | TTAGGAAAAGAAAAGACAATCA | |
| | | GAAATTTATTATCTTACAGCAA | |
| | | AGTAATGATAATTCTGTTCTTT | |
| | | ACAACATG | |
| | | GATACTATAAAATCTTTATCTT | |
| | | GGAATGTATTCTTTGAAATTAA | |
| | | ATGCATCAGAGCAATAATATAG | |
| | | TCTCCATGATGTTAGGATCATC | |
| | | CTTAGCAAAATTCTAATGGGAA | |
| | | ATGTAAATACCAGATATTTCTG | |
| | | TTTCCCAGTCATCTCTAAGACT | |
| | | GTATAACACAAGAAACTGGCTT | |
| | | ATTTGACATGGCTGATTAGACA | |
| | | GTCGTCTTAGGAAATACTTATT | |
| | | CATGGGGTGTTCTACCTGTTTT | |
| | | CCTTAAAC | |

SEQ ID NO: 460

| | | | |
|---|---|---|---|
| 353800 | SCAFFOLD | GTCTTTGCCCTTAACCATTATC | A/T |
| | 180695_15239 | CAAGAGGACCTCATGTCATTGT | |
| | | CCGACAGCTACCCAGCTATCAC | |
| | | ACATATTAAAATTAGCCCAATG | |
| | | TGCAATCAAGTCCATTATTTTT | |
| | | ATTTTTGTCAAATAGGGTTTTA | |
| | | AAGATTTGGTACATATATTTGA | |
| | | CCTTTTAGAAAGTTTCTTCTTC | |
| | | TTACTGAACACTGACTTTAATT | |
| | | ATTGAGGTACACTTCAGTTATG | |
| | | TTACCGTGTCTATTAAAAAAAC | |
| | | TTAACAAA | |
| | | ATTTTTGCAAAATAAATCTTGA | |
| | | CATACACTTTTCCTGTGTTCAG | |
| | | TTTAAAATTTTACTTCCTTCCT | |
| | | AAAACAATCTTGGTTCCTTCAT | |
| | | GATATAAATTTCCTTAAAGTTG | |
| | | GTGTTATCATGGCTTACCCAGA | |
| | | GCTAAAGATTATTTTTCTTTGT | |
| | | GACCGTCAGACATCAGGCATAA | |
| | | CTCGTAATGCAGAAGGCAAGCT | |
| | | ATCACAAACTGAGTTGAAGTAA | |
| | | GTTGTCAAATATGTGTTATTAG | |
| | | AAAGTCTC | |

SEQ ID NO: 461

| | | | |
|---|---|---|---|
| 353868 | SCAFFOLD | TCCCCGACCTGGGATCAAACCC | G/A |
| | 281020_16166 | AGGCCCCCTACATTTGGAGCAT | |
| | | GGAGTCTTAGCCCCTAGACTAC | |
| | | CAAGGAAATACCAATATGTGTG | |
| | | TAATTTTTAGCTTTAAATACTA | |
| | | GACGTCCTTGTCCGTATTTTAT | |
| | | CATCCTAATCTTTTTTTAGTGT | |
| | | ACTGCTTATAGAAATCACTAAT | |
| | | ATTCCTCTCGTCACCGGTGATG | |
| | | TGGAAAAAACATTTTGGTTTCT | |
| | | AACAGTTCATAGCACTTCTATT | |
| | | AATGCTTC | |
| | | TATCATAAGACAAAATATTAAA | |
| | | TTGAAATGAGAAAAAGGTGAAT | |
| | | GCCTAAGACCTGAACTGTTTTG | |
| | | CATACCTTGATCTCTCCATTGC | |
| | | AAGGTTACTTAGATGCTGGTGG | |
| | | GAGGAGGTTTAAATAGCTTTAA | |
| | | TAGTGCTGGTTTTATCAATTGC | |
| | | TGGTTATATCCATAACCTCATG | |
| | | GGCAATTGCTCAGTGAAAACCC | |
| | | AAGAAGCATTTTGTATCTATTT | |
| | | GAAACATACACACACTCATTTA | |
| | | AATACACA | |

SEQ ID NO: 462

| | | | |
|---|---|---|---|
| 353896 | SCAFFOLD | GCTGCCGTCTCTGGGGTTGCAC | T/C |
| | 35672_327 | AGAGTCGGACACGATTGAAGCG | |
| | | ACTTAGCAGCAGCAGGAACATA | |
| | | ACAGATAGTTCTCTGCCACTAT | |
| | | TCCAAAGTTTGACTCATAAGAA | |
| | | AGACCTAACAGAGTTTCTCTAT | |
| | | TATGCAAACATTCTCTGCAAAT | |
| | | GAGTCTGTCAAGGAAACATTTT | |
| | | TCCGAAAACTGTGTCTGTTAAC | |
| | | AGTGCTTGCATACTTACTTCTA | |
| | | CAATTTAGAATCTATTGTTTTT | |
| | | TCTCTACT | |
| | | GGTTTTTATTTTATGGTAATGT | |
| | | AGGCTTTTTCCAGTTAAAATAA | |
| | | TAAATACCTTTGTTATACTCAA | |
| | | AACATTCTTTATACATTAAATT | |
| | | GGTACAAATAACATAAAGTCTA | |
| | | TTTGAATTTAAAAATACAATTC | |
| | | ATTCTCTCATTACTACCCTGAC | |
| | | AAGTTACTAACATTTTAATTCA | |
| | | GAAAATAATTGATGTTATATAA | |
| | | GATTATTTACTTAACAATGCTT | |
| | | TGAAATTTAAAAAGCTGTATAC | |
| | | TATTTCAA | |

SEQ ID NO: 463

| | | | |
|---|---|---|---|
| 353926 | SCAFFOLD | GAAGGTATTGTTAGATATAAAT | T/C |
| | 70018_40019 | TGGATCATATATGAAGGAAGTT | |
| | | AAATGCCAAAGGAATTTTCCCA | |
| | | AGACTTTTCATTTTTCACTGAA | |
| | | CTCATCTAGGAAGGGAAAATAT | |
| | | GAAAGGATATAGGAAAATAATG | |
| | | TTGTGATGTTGATGCTTGTATT | |
| | | TAAAGGCAGAGAGAATTTAAGA | |
| | | CAACTTGTAATTTTGAACTAGA | |
| | | AACATTTATAAAGAAGCACTGA | |
| | | GGACCTAAAATTTGACATCAAA | |
| | | GGTAAATG | |
| | | TGAATGAAAATCTTAAAACCTT | |
| | | TTATAACTAATTATAATTTAGT | |
| | | ATCATCATGAGAAATTTGTATG | |
| | | CAGGTCAGGAAGCAACAGTTAG | |
| | | AATAGGGCATGGAACAACAGAC | |
| | | TTGTTCCAAATAGGAAAAGGAG | |
| | | TATGTCAAGGCTTGAGAACNNN | |
| | | NNNNNNNNNNNNNNNNNNNNNN | |
| | | NNNNNNNNNNNNNNNNNNNNNN | |
| | | NNNNNNNNNNNNNNNNNNNNNN | |
| | | NNNNNNNNNNNNNNNNNNNNNN | |
| | | NNNNNNNN | |

SEQ ID NO: 464

| | | | |
|---|---|---|---|
| 354055 | SCAFFOLD | GCAGTTTTACTGTCATGTGCCT | C/T |
| | 301712_11749 | AGAGGTAGTTTCTTTGTATTTA | |
| | | TTCTGTTTTGGATTCATAATTT | |
| | | ACTTTGAATATTAGGATAATCT | |
| | | CCTTCATTAATTTTGAAAAATG | |
| | | ATTGGCTGCTCTTTTCAAATAT | |
| | | TGTTCCGTCTCCTTTTCTCTCT | |
| | | CCTTTCCAAGGGGAACCCTATT | |
| | | GCATATATCTTAGACCTTTTAT | |
| | | CTATGTCATCTATGTTTCTTAT | |
| | | GATCATCTCTGTTTTTTGATAA | |
| | | TTTTATTA | |
| | | TGGTTAGTTCTAATCATTACTT | |
| | | TCCATGCCTTGAACTGGCCAAA | |
| | | TCTCCCCTCACCTCAGTGCCTG | |
| | | TACACGTGCTCTGTCTGGGTCA | |
| | | CGGTCTCCCTTTACCTTGCCTT | |
| | | TGCCTTAAGTCACTTATTATTC | |
| | | AAGTCTCGGCTTAGCTTAACTC | |
| | | TCTCTAGGAAACCTTTTATGAC | |
| | | CCCTAAATTCTTGCCTAGATTC | |
| | | AGAGTACTGTGTTTTTTCCTCA | |
| | | GTGATATTTCTTATCCCCCTCA | |
| | | TATTCAAAA | |

SEQ ID NO: 465

| | | | |
|---|---|---|---|
| 354135 | SCAFFOLD | TTCTAAGTAATTCCTTTCAAGA | A/T |
| | 155226_731 | AATCAAAATTGAAAGTATAAGC | |
| | | TCTTTAGGACTGAATGACAGTG | |
| | | GGAATATTGAATACAAATAACT | |
| | | ATGAAATTCAGCTACAGTCATA | |
| | | CTCTGGAGAAAATTCAGAATTT | |
| | | AATGCCTTTGTTATTACACAAG | |
| | | AGACTAAGCATTCAACTCACTC | |
| | | ACATAATAAAAATAACACCATA | |
| | | AGAAATCTAAAGAAAGGAAAAG | |
| | | GATATAATAAATATAAAACTAG | |
| | | AAATCAAT | |
| | | GATAAGCAAGCAAAAGTAGAAA | |
| | | AATAATGGATAAAACTAAGAAT | |
| | | CTGACCTCTGGTGGGAGTGAAG | |
| | | GGATTAATAACAGATACATTTT | |
| | | TAGAAAAACAAATTGGTTACCA | |
| | | AGTTCACCTGAAAGAACAAATA | |
| | | AAGACCATTGGTTAAAAATGCA | |
| | | AGACTGAAAACATGCATTTAAC | |
| | | CTTCACTCTCTCCTGAAATCCT | |
| | | ACAAAAATGAGAATAAAGGTAT | |
| | | TTTTCTCTTTGTTTGATTTTTG | |
| | | CGTAAATT | |

SEQ ID NO: 466

| | | | |
|---|---|---|---|
| 354174 | SCAFFOLD | AGTATCTTGGTTGAGACAGGAA | G/T |
| | 260431_8099 | TGACAACCATGCCTGCTTGTCA | |
| | | GAGTTTTAAACTTTGGAGACCA | |
| | | AAATAGGCTTTATAAGCTGCCA | |
| | | CTAAAAAGAACAGATAAGATTT | |
| | | AAATACACAATGAGAAGATAAG | |
| | | AATCAACTTGGACCATCCTCCT | |
| | | CAGACTTGGGCAAAGCTAAAAT | |
| | | AAGTGGAAATATCACCGCAAGA | |
| | | ACAGTGAGGCAAGCTGACTGGG | |
| | | GAGAAGAAAAACATTTTAAAAG | |
| | | TATGAGAC | |
| | | ATATATTTAAAAAACTGTCAAA | |
| | | GAAGCCTCAAATTAAAGATCCC | |
| | | AAGAAGAAAATAAGGAGATCTA | |
| | | GAAGTAGATGAACTAGAGAAAA | |
| | | AAAGCGGAAATAGATTAAGAAG | |
| | | GTGAAGACAAAGAAGTGGATTC | |
| | | TATATCAAAGGGGGAAAATTGA | |
| | | TAAGTTAAACAAAATAGTATGA | |
| | | AAACTACAGGGTAAAATATAAA | |
| | | AATAATAGAAGAAAATCACATC | |
| | | TTAATTGATAATATGTCTGGAA | |
| | | TGCAGACT | |

SEQ ID NO: 467

| | | | |
|---|---|---|---|
| 354182 | SCAFFOLD | GAGCTCAACCTAAAATGAAAAG | C/T |
| | 280379_12043 | TTATGCATCCAGTAAATTTTTC | |
| | | AAGAGGCAGTTCATTTTTATGC | |
| | | TTATTATTAACTTTCATTATAT | |
| | | TCATTAAAATGTTCAACTTTAT | |
| | | CCTAAATCACCTAAATCTTCCA | |
| | | TGAGCTCTGCATGTGGAAAAGA | |
| | | AGTTGGCAGATGCTTAATTTGG | |
| | | AGAAATTGCTTACTTTTGCATA | |
| | | AAACTAATTTTTGTAAGAGCCA | |
| | | GGAAAATATTAGAAGAAATTAT | |
| | | TTATGTGC | |
| | | TAAAAATTCAAACATTTTGGTT | |
| | | TTGAACAGAGGCATAAAGAGAA | |
| | | TACATCTCATATACTTTGGAAG | |
| | | AAAGCCTCAGATGTATGAAACT | |
| | | CAAAAGGAATGAATAATAGCAA | |
| | | CTTATTTGCTGGGCAGGAGTCA | |
| | | ATCAATTCTCACTTATTTAATG | |
| | | AGTTAGGAAACAGAATGAAGGT | |
| | | GGGGTAATTTTCAAAACACATC | |
| | | TTCCACAGTCCTTGTTTACAGT | |
| | | ATATTTATAATTTACCTGGGAA | |
| | | TACAAGGC | |

SEQ ID NO: 468

| | | | |
|---|---|---|---|
| 354184 | SCAFFOLD | TTGATTGAATTTGCTCAAAAGA | A/C |
| | 285028_33668 | AGATAGCTGCACTGTTAATCAT | |
| | | ATTTATTAGTTGATCTTAAGAA | |
| | | TTCATGCATTTAATTGTTCTAG | |
| | | AATTAGAGATAAACCTTGGAAT | |
| | | TATGACTGTGAGAAATGGCCAA | |
| | | GAGTTTCATTGGGGATGACAGA | |
| | | AGGCAATGCTGAGCAAGAAGGA | |
| | | GGTTTGAAATCCTGCATTAATT | |
| | | CTAGAGTTCATCAACCTATGAC | |
| | | TATGGAAATCTCGATATATTTG | |
| | | AATTGCAA | |
| | | AATTTATAAAAAACTCTTAAAT | |
| | | ACTTTTTGTCCTTCTGTTTTCT | |
| | | CCTTCAATATAAGCCTACAAAA | |
| | | TTATAAGGTACTTAAGTGTTGG | |
| | | GACCATGTCTCTGAATCTTTAA | |
| | | TAACAAACACACTCTCATAGTA | |
| | | GGTGTCCAAAACTATATTATTG | |
| | | ATTGTTTTATATGTGAGTTGGA | |
| | | GAAAGATGCATAAAAATGTCTA | |
| | | AGCATGCTAGAATTCTCTTTAT | |
| | | TTATTTTTTGGCTGCACCACTC | |
| | | AGCTTCCA | |

SEQ ID NO: 469

| | | | |
|---|---|---|---|
| 354282 | SCAFFOLD | TAAATTGCTAAGTCAAAATGAC | T/C |
| | 195918_13722 | AATATTTTGAATAAAATTTATA | |
| | | TAAATTATGATGCCATAAAAGT | |
| | | ATATTATTTTTAAAATTGAATT | |
| | | AGTAATTTCATAACAATCACTA | |
| | | ATTAGTGATTTTACAGCTGAGT | |
| | | GTTAGTCATTCTTCCATACTCT | |
| | | GTCTGCCTTTCTTGCACTTAAA | |
| | | ATGTCTATTTGTCTAATTGGAG | |
| | | CTTTTTGTTCTAATTTGGGATA | |
| | | ATATCATTAACTTTTTAAAATT | |
| | | CTTCACCA | |
| | | GTTTTAATGATCAATTACTTTA | |
| | | TATTCTTAAATGTTAGTTACAA | |
| | | TAATATTTTACTCACAAATTTC | |
| | | CATTGAAAATTTTCATGTAAAA | |
| | | TAACTCCAGCAAATATTAACAT | |
| | | GGCTCATTTTACCCAAGCAATT | |
| | | GTGCTTTCATTTCTTTTATATT | |
| | | GAGAGATAGTATAGCCCACGAA | |
| | | GGACACAGACTCTGGCATCAGA | |
| | | CTTTCTGCATTCAATCTGTTTG | |
| | | CAGCCATATAAATATGTGACCT | |
| | | TGAACAAA | |

SEQ ID NO: 470

| | | | |
|---|---|---|---|
| 354414 | SCAFFOLD | ATCTAGAACACAATCTTTAACC | C/T |
| | 120581_8000 | TAGTAGTAATTTTTCTTCAATA | |
| | | GATGTAAATGCAAATTTGAGAA | |
| | | AAAATTCAAATATTAGTTGGTT | |
| | | AACGTCTCATAGAATGCATAAT | |
| | | TAATCTAAGTCTAAAATATGTA | |
| | | AACTTAGAAATTCTGAATCAAT | |
| | | TGATCATTGATATTACTAGAAA | |
| | | ATGTTTAATTATGTAAGCAATT | |
| | | CTTTGTTAACTGAAAGGATTTC | |
| | | ACTTTTTAAAAATATCTATATC | |
| | | TTTTTCTT | |
| | | CATAATTTTAAGCAAAATTTTC | |
| | | ATAACAAATAATTTATTTTTGT | |
| | | CCTCTATCCATATAATTATAAT | |
| | | ATTTTATTATTTTGAATGAATT | |
| | | TTGAAGGTTAAACATTTCAATG | |
| | | AAAATTAAACAACTGGATCTGC | |
| | | ACACAGCGAAGCTATTGCCCGA | |
| | | GGTATCTGGTGTACTGTTACAA | |
| | | GGAAGTTTTATGATATGTATTG | |
| | | TTTATCTCAACCTACATCTTAC | |
| | | CATATCTTAGATGTCTGCTAAC | |
| | | TAAGATTA | |

SEQ ID NO: 471

| | | | |
|---|---|---|---|
| 354455 | SCAFFOLD | TCAACGGACATAAATTTGAGCA | C/T |
| | 334643_69 | AACTCCAGGAGACAGTGAAGGA | |
| | | CAGGGAAGCCGGGTGTGCTGCA | |
| | | GTTCATGGGGTTGCAACAACTC | |
| | | GGATACAACTTAGAGACTGAAC | |
| | | AACAACATATGGCAGAAGAAAA | |
| | | TAACTGATCATTCTAGCTGCAT | |
| | | TCATAATAAAATTTGAGATGAG | |
| | | AAATTTGATCATTTGAGATGGC | |
| | | CATGGGAATGGATTCAATTATA | |
| | | GCTAAAAATCTACTAAATATCT | |
| | | ACTACATA | |
| | | CTATGTTTTTAAAAATGTGTTA | |
| | | TAACTGAAGGTTTCTTTGGGTA | |
| | | AAGCAACTAGATTGTCCTGTCC | |
| | | AC | |

TABLE 18
Further SNP associated with intramuscular fat
deposition

| Ident | Scaffold | SEQ ID NO: |
|---|---|---|
| 342547 | SCAFFOLD220683_1125 | 472 |
| 342599 | SCAFFOLD312276_9056 | 473 |
| 342822 | SCAFFOLD115102_212 | 474 |
| 342867 | SCAFFOLD150792_11786 | 475 |
| 342874 | SCAFFOLD152075_3289 | 476 |
| 342901 | SCAFFOLD170266_2103 | 477 |
| 342907 | SCAFFOLD180556_11337 | 478 |
| 342939 | SCAFFOLD205027_17860 | 479 |
| 342957 | SCAFFOLD220223_540 | 480 |
| 343085 | SCAFFOLD61841_3624 | 481 |
| 343112 | SCAFFOLD92148_5123 | 482 |
| 343173 | IBISS4snp406 | 483 |
| 343273 | SCAFFOLD128805_3090 | 484 |
| 343365 | SCAFFOLD17903_1700 | 485 |
| 343384 | SCAFFOLD190167_18200 | 486 |
| 343386 | SCAFFOLD190221_16495 | 487 |
| 343403 | SCAFFOLD199393_2655 | 489 |
| 343487 | SCAFFOLD25039_6855 | 490 |
| 343513 | SCAFFOLD263303_2666 | 491 |
| 343584 | SCAFFOLD310380_2422 | 492 |
| 343688 | SCAFFOLD70184_5981 | 493 |
| 343714 | SCAFFOLD85708_9285 | 494 |
| 343858 | SCAFFOLD116206_487 | 495 |
| 343861 | SCAFFOLD1191_3524 | 496 |
| 343882 | SCAFFOLD125974_8130 | 497 |
| 343947 | SCAFFOLD155540_1585 | 498 |
| 343966 | SCAFFOLD165210_21228 | 499 |
| 343979 | SCAFFOLD170136_45658 | 500 |
| 343981 | SCAFFOLD170266_2028 | 501 |
| 343982 | SCAFFOLD170266_1888 | 502 |
| 344020 | SCAFFOLD190067_20138 | 503 |
| 344033 | SCAFFOLD195717_4245 | 504 |
| 344061 | SCAFFOLD206794_1935 | 505 |
| 344070 | SCAFFOLD213107_3591 | 506 |
| 344073 | SCAFFOLD215465_13068 | 507 |
| 344120 | SCAFFOLD240740_10374 | 508 |
| 344165 | SCAFFOLD260597_2435 | 509 |
| 344242 | SCAFFOLD295231_20757 | 510 |
| 344268 | SCAFFOLD308591_553 | 511 |
| 344298 | SCAFFOLD321801_15731 | 512 |
| 344342 | SCAFFOLD46484_3279 | 513 |
| 344344 | SCAFFOLD46716_2213 | 514 |
| 344362 | SCAFFOLD56714_469 | 515 |
| 344373 | SCAFFOLD60223_13631 | 516 |
| 344428 | SCAFFOLD86623_4947 | 517 |
| 344465 | IBISS4snp14 | 518 |
| 344472 | IBISS4snp154 | 519 |
| 344501 | IBISS4snp635 | 520 |
| 344568 | SCAFFOLD110655_10863 | 521 |
| 344576 | SCAFFOLD115003_498 | 522 |
| 344596 | SCAFFOLD125560_2483 | 523 |
| 344655 | SCAFFOLD150141_9327 | 524 |
| 344673 | SCAFFOLD155453_11913 | 525 |
| 344679 | SCAFFOLD156398_6086 | 526 |
| 344698 | SCAFFOLD165090_26679 | 527 |
| 344702 | SCAFFOLD170136_45529 | 528 |
| 344729 | SCAFFOLD185014_39638 | 529 |
| 344738 | SCAFFOLD190067_20228 | 530 |
| 344787 | SCAFFOLD206273_12652 | 531 |
| 344805 | SCAFFOLD216235_3404 | 532 |
| 344814 | SCAFFOLD220223_2601 | 533 |
| 344847 | SCAFFOLD230250_13663 | 534 |
| 344874 | SCAFFOLD240305_16330 | 535 |
| 344916 | SCAFFOLD25048_12981 | 536 |
| 344932 | SCAFFOLD255096_4540 | 537 |
| 344950 | SCAFFOLD2641_4426 | 538 |
| 344998 | SCAFFOLD282025_3342 | 539 |
| 345005 | SCAFFOLD285987_6043 | 540 |
| 345016 | SCAFFOLD291452_7280 | 541 |
| 345024 | SCAFFOLD295336_4564 | 542 |
| 345043 | SCAFFOLD300283_17094 | 543 |
| 345048 | SCAFFOLD303315_5993 | 544 |
| 345050 | SCAFFOLD30496_3799 | 545 |
| 345142 | SCAFFOLD55505_8936 | 546 |
| 345188 | SCAFFOLD70177_16365 | 547 |
| 345229 | SCAFFOLD83266_1276 | 548 |
| 345252 | SCAFFOLD90222_4388 | 549 |
| 345359 | SCAFFOLD106798_1888 | 550 |
| 345430 | SCAFFOLD135102_4826 | 551 |
| 345473 | SCAFFOLD15286_17074 | 552 |
| 345586 | SCAFFOLD186010_8596 | 553 |
| 345617 | SCAFFOLD201590_8431 | 554 |
| 345639 | SCAFFOLD210206_22955 | 555 |
| 345762 | SCAFFOLD25039_13622 | 556 |
| 345764 | SCAFFOLD25056_13176 | 557 |
| 345765 | SCAFFOLD25056_13047 | 558 |
| 345930 | SCAFFOLD308591_997 | 559 |
| 345974 | SCAFFOLD323635_4515 | 560 |
| 346001 | SCAFFOLD40330_13030 | 561 |
| 346006 | SCAFFOLD40979_2070 | 562 |
| 346122 | SCAFFOLD80060_16638 | 563 |
| 346157 | SCAFFOLD95077_5259 | 564 |
| 346192 | IBISS4snp499 | 565 |
| 346260 | SCAFFOLD110655_10681 | 566 |
| 346298 | SCAFFOLD130523_7121 | 567 |
| 346304 | SCAFFOLD131537_5113 | 568 |
| 346308 | SCAFFOLD135027_35999 | 569 |
| 346313 | SCAFFOLD136321_685 | 570 |
| 346314 | SCAFFOLD136467_4130 | 571 |
| 346369 | SCAFFOLD155993_1701 | 572 |
| 346380 | SCAFFOLD160202_23833 | 573 |
| 346419 | SCAFFOLD170266_2076 | 574 |
| 346467 | SCAFFOLD185315_13273 | 575 |
| 346575 | SCAFFOLD227097_1473 | 576 |
| 346587 | SCAFFOLD231905_9561 | 577 |
| 346612 | SCAFFOLD245097_12680 | 578 |
| 346615 | SCAFFOLD245243_1474 | 579 |
| 346635 | SCAFFOLD25039_13816 | 580 |
| 346674 | SCAFFOLD265658_20008 | 581 |
| 346676 | SCAFFOLD266100_4197 | 582 |
| 346705 | SCAFFOLD275389_15487 | 523 |
| 346809 | SCAFFOLD311747_37269 | 584 |
| 346870 | SCAFFOLD35718_5721 | 585 |
| 346914 | SCAFFOLD50887_11878 | 586 |
| 346945 | SCAFFOLD60682_7938 | 587 |
| 346988 | SCAFFOLD79504_1545 | 588 |
| 347050 | IBISS4snp483 | 589 |
| 347113 | SCAFFOLD106320_3036 | 590 |
| 347158 | SCAFFOLD126125_3685 | 591 |
| 347188 | SCAFFOLD136321_994 | 592 |
| 347218 | SCAFFOLD150215_5759 | 593 |
| 347227 | SCAFFOLD152016_5940 | 594 |
| 347228 | SCAFFOLD152016_5856 | 595 |
| 347231 | SCAFFOLD153002_7959 | 596 |
| 347248 | SCAFFOLD15986_9482 | 597 |
| 347291 | SCAFFOLD170799_16306 | 598 |
| 347326 | SCAFFOLD185336_7376 | 599 |
| 347384 | SCAFFOLD20573_13572 | 600 |
| 347385 | SCAFFOLD20573_13420 | 601 |
| 347424 | SCAFFOLD216837_2933 | 602 |
| 347434 | SCAFFOLD220021_8153 | 603 |
| 347440 | SCAFFOLD220733_6491 | 604 |
| 347461 | SCAFFOLD227180_6296 | 605 |
| 347501 | SCAFFOLD245021_7420 | 606 |
| 347533 | SCAFFOLD25332_9402 | 607 |
| 347599 | SCAFFOLD270624_13061 | 608 |
| 347606 | SCAFFOLD275_9222 | 609 |
| 347635 | SCAFFOLD281900_1531 | 610 |
| 347665 | SCAFFOLD295336_4448 | 611 |
| 347681 | SCAFFOLD300039_11689 | 612 |
| 347684 | SCAFFOLD300283_16976 | 613 |
| 347760 | SCAFFOLD337715_136 | 614 |
| 347761 | SCAFFOLD340752_551 | 615 |
| 347806 | SCAFFOLD46392_11621 | 616 |
| 347818 | SCAFFOLD50170_9319 | 617 |
| 347866 | SCAFFOLD65622_9619 | 618 |
| 347884 | SCAFFOLD70184_5962 | 619 |
| 347915 | SCAFFOLD80895_16576 | 620 |
| 347933 | SCAFFOLD87_22677 | 621 |
| 348058 | SCAFFOLD107820_1332 | 622 |
| 348065 | SCAFFOLD110184_3008 | 623 |
| 348104 | SCAFFOLD120672_10956 | 624 |
| 348149 | SCAFFOLD135282_16356 | 625 |
| 348151 | SCAFFOLD136321_1119 | 626 |
| 348152 | SCAFFOLD136321_807 | 627 |
| 348166 | SCAFFOLD140224_4081 | 628 |
| 348201 | SCAFFOLD152968_3124 | 629 |
| 348215 | SCAFFOLD155509_6872 | 630 |
| 348228 | SCAFFOLD160090_6137 | 631 |
| 348231 | SCAFFOLD160889_3126 | 632 |
| 348232 | SCAFFOLD160889_3290 | 633 |
| 348353 | SCAFFOLD206920_7355 | 634 |
| 348383 | SCAFFOLD216837_2774 | 635 |
| 348421 | SCAFFOLD230819_6392 | 636 |
| 348445 | SCAFFOLD240220_8809 | 637 |
| 348462 | SCAFFOLD246753_1482 | 638 |
| 348518 | SCAFFOLD265658_20627 | 639 |
| 348528 | SCAFFOLD268164_2677 | 640 |
| 348573 | SCAFFOLD285028_33324 | 641 |
| 348625 | SCAFFOLD303315_6102 | 642 |
| 348636 | SCAFFOLD306219_3093 | 643 |
| 348673 | SCAFFOLD322772_7036 | 644 |
| 348693 | SCAFFOLD36_14443 | 645 |
| 348726 | SCAFFOLD46484_3075 | 646 |
| 348753 | SCAFFOLD56714_336 | 647 |
| 348760 | SCAFFOLD57440_1204 | 648 |
| 348766 | SCAFFOLD60231_15046 | 649 |
| 348888 | IBISS4snp785 | 650 |
| 348912 | SCAFFOLD10204_22524 | 651 |
| 348933 | SCAFFOLD110132_5363 | 652 |
| 348940 | SCAFFOLD110655_10917 | 653 |
| 349013 | SCAFFOLD1401_4286 | 654 |
| 349048 | SCAFFOLD151694_4517 | 655 |
| 349070 | SCAFFOLD155739_12976 | 656 |
| 349103 | SCAFFOLD170266_1792 | 657 |
| 349144 | SCAFFOLD183469_2141 | 658 |
| 349152 | SCAFFOLD186010_9791 | 659 |
| 349153 | SCAFFOLD186028_9587 | 660 |
| 349197 | SCAFFOLD199519_2524 | 661 |
| 349256 | SCAFFOLD220358_6604 | 662 |
| 349258 | SCAFFOLD220986_9511 | 663 |
| 349265 | SCAFFOLD221810_557 | 664 |
| 349305 | SCAFFOLD231881_542 | 665 |
| 349306 | SCAFFOLD232808_795 | 666 |
| 349345 | SCAFFOLD250116_14342 | 667 |
| 349347 | SCAFFOLD250454_7761 | 668 |
| 349413 | SCAFFOLD27004_5586 | 669 |
| 349442 | SCAFFOLD280364_1108 | 670 |
| 349547 | SCAFFOLD313308_208 | 671 |
| 349571 | SCAFFOLD323674_1570 | 672 |
| 349580 | SCAFFOLD351875_395 | 673 |
| 349594 | SCAFFOLD40058_29791 | 674 |
| 349614 | SCAFFOLD5008_24057 | 675 |
| 349618 | SCAFFOLD50419_5418 | 676 |
| 349672 | SCAFFOLD70018_37068 | 677 |
| 349673 | SCAFFOLD70018_41330 | 678 |
| 349717 | SCAFFOLD90222_4848 | 679 |
| 349730 | SCAFFOLD92061_5686 | 680 |
| 349735 | SCAFFOLD95032_12466 | 681 |
| 349751 | IBISS4snp26 | 682 |
| 349777 | IBISS4snp1021 | 683 |
| 349814 | SCAFFOLD110929_383 | 684 |
| 349822 | SCAFFOLD115093_14103 | 685 |
| 349875 | SCAFFOLD135046_8927 | 686 |
| 349919 | SCAFFOLD150945_2291 | 687 |
| 349942 | SCAFFOLD158550_8551 | 688 |
| 349966 | SCAFFOLD170429_18521 | 689 |
| 349970 | SCAFFOLD172341_13427 | 690 |
| 350042 | SCAFFOLD195740_9619 | 691 |
| 350057 | SCAFFOLD201176_15817 | 692 |
| 350072 | SCAFFOLD205706_3317 | 693 |
| 350091 | SCAFFOLD211870_1750 | 694 |
| 350161 | SCAFFOLD245471_31606 | 695 |
| 350217 | SCAFFOLD265091_15662 | 696 |
| 350228 | SCAFFOLD265658_20099 | 697 |
| 350229 | SCAFFOLD265658_20229 | 698 |
| 350254 | SCAFFOLD275678_3653 | 699 |
| 350276 | SCAFFOLD281988_2093 | 700 |
| 350348 | SCAFFOLD311898_4113 | 701 |
| 350385 | SCAFFOLD323635_4462 | 702 |
| 350386 | SCAFFOLD323674_1755 | 703 |
| 350404 | SCAFFOLD36124_6205 | 704 |
| 350425 | SCAFFOLD46484_3384 | 705 |
| 350426 | SCAFFOLD46484_3171 | 706 |
| 350446 | SCAFFOLD52080_7447 | 707 |
| 350451 | SCAFFOLD55461_687 | 708 |
| 350468 | SCAFFOLD61089_3325 | 709 |
| 350535 | SCAFFOLD81100_3471 | 710 |
| 350542 | SCAFFOLD85037_17745 | 711 |
| 350557 | SCAFFOLD90038_35582 | 712 |
| 350617 | SCAFFOLD101114_2006 | 713 |
| 350637 | SCAFFOLD106099_7340 | 714 |
| 350638 | SCAFFOLD106320_2885 | 715 |
| 350677 | SCAFFOLD125539_11512 | 716 |
| 350739 | SCAFFOLD152968_3192 | 717 |
| 350759 | SCAFFOLD160575_768 | 718 |
| 350773 | SCAFFOLD1654_874 | 719 |
| 350818 | SCAFFOLD185976_2662 | 720 |
| 350827 | SCAFFOLD190252_3148 | 721 |
| 350881 | SCAFFOLD215406_5701 | 722 |
| 350943 | SCAFFOLD246578_8558 | 723 |
| 351077 | SCAFFOLD30030_6703 | 724 |
| 351110 | SCAFFOLD316708_43326 | 725 |
| 351212 | SCAFFOLD56714_405 | 726 |
| 351265 | SCAFFOLD80180_17284 | 727 |
| 351278 | SCAFFOLD85279_22726 | 728 |
| 351310 | SCAFFOLD96480_845 | 729 |
| 351316 | IBISS4snp40 | 730 |
| 351373 | SCAFFOLD110929_430 | 731 |
| 351471 | SCAFFOLD165946_4962 | 732 |
| 351503 | SCAFFOLD186010_8404 | 733 |
| 351507 | SCAFFOLD186587_2456 | 734 |
| 351538 | SCAFFOLD20183_16424 | 735 |
| 351548 | SCAFFOLD206776_2497 | 736 |
| 351603 | SCAFFOLD240158_17269 | 737 |
| 351612 | SCAFFOLD245219_2073 | 738 |
| 351675 | SCAFFOLD270264_3928 | 739 |
| 351745 | SCAFFOLD302849_679 | 740 |
| 351762 | SCAFFOLD312013_3173 | 741 |
| 351772 | SCAFFOLD313610_19168 | 742 |
| 351780 | SCAFFOLD316906_13351 | 743 |
| 351815 | SCAFFOLD367946_446 | 744 |
| 351834 | SCAFFOLD46184_583 | 745 |
| 351869 | SCAFFOLD60723_4011 | 746 |
| 351893 | SCAFFOLD75014_22303 | 747 |
| 351977 | SCAFFOLD110052_16832 | 748 |
| 351995 | SCAFFOLD115297_3807 | 749 |
| 351998 | SCAFFOLD11903_9629 | 750 |
| 352051 | SCAFFOLD135046_16431 | 751 |
| 352053 | SCAFFOLD136467_7319 | 752 |
| 352088 | SCAFFOLD161539_2467 | 753 |
| 352103 | SCAFFOLD166319_684 | 754 |
| 352149 | SCAFFOLD195953_12294 | 755 |
| 352177 | SCAFFOLD210729_20981 | 756 |
| 352208 | SCAFFOLD230061_15292 | 757 |
| 352232 | SCAFFOLD245471_32043 | 758 |
| 352323 | SCAFFOLD291797_4042 | 759 |
| 352334 | SCAFFOLD299122_4777 | 760 |
| 352337 | SCAFFOLD301689_4716 | 761 |
| 352342 | SCAFFOLD305434_14153 | 762 |
| 352360 | SCAFFOLD312547_2687 | 763 |
| 352387 | SCAFFOLD37186_1033 | 764 |
| 352407 | SCAFFOLD46184_706 | 765 |
| 352421 | SCAFFOLD55194_29776 | 766 |
| 352426 | SCAFFOLD6048_1026 | 767 |
| 352427 | SCAFFOLD60489_16555 | 768 |
| 352458 | SCAFFOLD75897_13740 | 769 |
| 352524 | SCAFFOLD103209_657 | 770 |
| 352532 | SCAFFOLD110141_21677 | 771 |
| 352560 | SCAFFOLD125693_15530 | 772 |
| 352564 | SCAFFOLD130277_4678 | 773 |
| 352578 | SCAFFOLD136998_2601 | 774 |
| 352587 | SCAFFOLD144700_3565 | 775 |
| 352640 | SCAFFOLD170799_16678 | 776 |
| 352695 | SCAFFOLD200631_515 | 777 |
| 352804 | SCAFFOLD270626_18710 | 778 |
| 352828 | SCAFFOLD285972_2879 | 779 |
| 352888 | SCAFFOLD31598_11367 | 780 |
| 352912 | SCAFFOLD40294_36611 | 781 |
| 352920 | SCAFFOLD46484_3524 | 782 |
| 352928 | SCAFFOLD5178_6226 | 783 |
| 352932 | SCAFFOLD55068_10779 | 784 |
| 352966 | SCAFFOLD76779_3070 | 785 |
| 352986 | SCAFFOLD86113_4825 | 786 |
| 353009 | IBISS4snp454 | 787 |
| 353020 | SCAFFOLD100651_19313 | 788 |
| 353031 | SCAFFOLD105204_16908 | 789 |
| 353070 | SCAFFOLD130352_25837 | 790 |
| 353084 | SCAFFOLD144700_3610 | 791 |
| 353126 | SCAFFOLD169748_4521 | 792 |
| 353131 | SCAFFOLD171003_4691 | 793 |
| 353148 | SCAFFOLD189868_810 | 794 |
| 353187 | SCAFFOLD216959_939 | 795 |
| 353209 | SCAFFOLD240356_5849 | 796 |
| 353211 | SCAFFOLD240435_14381 | 797 |
| 353215 | SCAFFOLD245166_13909 | 798 |
| 353221 | SCAFFOLD246578_8403 | 799 |
| 353222 | SCAFFOLD246578_8740 | 800 |
| 353250 | SCAFFOLD261939_875 | 801 |
| 353319 | SCAFFOLD316906_13568 | 802 |
| 353335 | SCAFFOLD35042_1845 | 803 |
| 353346 | SCAFFOLD40955_7411 | 804 |
| 353416 | IBISS4snp616 | 805 |
| 353421 | IBISS4snp639 | 806 |
| 353479 | SCAFFOLD142117_3231 | 807 |
| 353491 | SCAFFOLD151966_3291 | 808 |
| 353496 | SCAFFOLD155611_5257 | 809 |
| 353521 | SCAFFOLD181031_12304 | 810 |
| 353573 | SCAFFOLD242802_999 | 811 |
| 353575 | SCAFFOLD245284_14755 | 812 |
| 353784 | SCAFFOLD160922_9412 | 813 |
| 353791 | SCAFFOLD174893_838 | 814 |
| 353817 | SCAFFOLD205195_11372 | 815 |
| 353840 | SCAFFOLD245166_13946 | 816 |
| 353863 | SCAFFOLD275520_5504 | 817 |
| 353891 | SCAFFOLD322772_7012 | 818 |
| 353905 | SCAFFOLD40955_6948 | 819 |
| 353918 | SCAFFOLD6048_1174 | 820 |
| 353923 | SCAFFOLD65152_4915 | 821 |
| 353927 | SCAFFOLD70323_17093 | 822 |
| 353929 | SCAFFOLD71223_1675 | 823 |
| 353944 | SCAFFOLD95088_21248 | 824 |
| 354008 | SCAFFOLD190005_17173 | 825 |
| 354028 | SCAFFOLD245012_39265 | 826 |
| 354036 | SCAFFOLD262092_7605 | 827 |
| 354068 | SCAFFOLD322012_24590 | 828 |
| 354108 | SCAFFOLD100305_20665 | 829 |
| 354153 | SCAFFOLD200090_9837 | 830 |
| 354175 | SCAFFOLD262092_7358 | 831 |
| 354213 | SCAFFOLD55125_32713 | 832 |
| 354228 | SCAFFOLD75727_10291 | 833 |
| 354281 | SCAFFOLD19541_879 | 834 |
| 354317 | SCAFFOLD308591_905 | 835 |
| 354346 | SCAFFOLD130137_7497 | 836 |
| 354353 | SCAFFOLD162128_4762 | 837 |
| 354375 | SCAFFOLD26010_1466 | 838 |
| 354395 | SCAFFOLD56872_6521 | 839 |
| 354476 | SCAFFOLD286022_2861 | 840 |
| 354494 | SCAFFOLD125474_7234 | 841 |
| 354536 | SCAFFOLD40159_1874 | 842 |

### EXAMPLE 6

An analysis of the trait of retail beef yield (RBY) was performed on the same animals as in previous examples and employing the methodology described therein. The analysis performed directly on RBY was compared to an analysis performed on a combination of P8 fat and carcass weight, to determine if there was any commonality in the SNP identified via the two methods.

TABLE 19
Associations between DNA markers and retail beef yield sorted in
decreasing order of statistical significance. Locus and ident are the ParAllele
identifiers of the polymorphisms, inf is whether there are enough genotypes for
each genotype for a proper test, N is the number of genotypes, mean_0 is the mean
total bone out retail beef yield for genotype 0, mean_1 is the mean for genotype 1,
and mean_2 is the mean of genotype 2, SD is the standard deviation, maxd is the
maximum difference between any of the three genotypes, t is the value of the t
test, P is the P value determined from 100,000 permutation tests and log(1/P) is
the P value expressed as a positive integer.

| locus | ident | inf | N | mean_0 | SD | N | mean_1 | SD | N | mean_2 | SD | maxd | t | P | log(1/P) | SEQ ID NO: |
|---|---|---|---|---|---|---|---|---|---|---|---|---|---|---|---|---|
| 6475 | 347657 | Y | 38 | 0.60 | 2.98 | 23 | 0.91 | 2.21 | 11 | −3.49 | 2.01 | 4.397 | 8.435 | 0.000E+00 | 5.000 | 843 |
| 1297 | 345212 | Y | 26 | 1.39 | 2.22 | 33 | 0.20 | 3.04 | 13 | −2.86 | 2.35 | 4.247 | 7.776 | 0.000E+00 | 5.000 | 844 |
| 4931 | 353110 | Y | 12 | −2.68 | 2.52 | 15 | 1.78 | 1.98 | 44 | 0.31 | 2.94 | 4.463 | 7.787 | 0.300E−04 | 4.523 | 845 |
| 518 | 351736 | Y | 14 | −2.78 | 2.71 | 26 | 0.17 | 2.58 | 32 | 1.25 | 2.68 | 4.029 | 6.957 | 0.300E−04 | 4.523 | 846 |
| 7268 | 350185 | Y | 43 | 0.58 | 2.81 | 25 | 0.07 | 2.77 | 4 | −5.29 | 0.82 | 5.866 | 12.540 | 0.500E−04 | 4.301 | 847 |
| 3645 | 353986 | Y | 37 | 0.51 | 3.01 | 29 | −1.08 | 2.78 | 6 | 2.96 | 1.07 | 4.036 | 8.348 | 0.140E−03 | 3.854 | 848 |
| 3517 | 353512 | Y | 26 | −1.80 | 2.87 | 31 | 0.85 | 2.58 | 15 | 1.73 | 2.49 | 3.535 | 6.306 | 0.190E−03 | 3.721 | 849 |
| 1256 | 345055 | Y | 0 | 0.00 | 0.00 | 3 | −4.41 | 0.20 | 69 | 0.27 | 2.92 | 4.675 | 17.850 | 0.220E−03 | 3.658 | 850 |
| 7756 | 351887 | Y | 42 | 0.53 | 2.96 | 14 | 1.42 | 2.48 | 16 | −2.30 | 2.32 | 3.718 | 6.490 | 0.250E−03 | 3.602 | 851 |
| 6849 | 348851 | Y | 36 | 1.06 | 2.85 | 25 | −0.02 | 2.57 | 11 | −2.93 | 2.56 | 3.992 | 6.637 | 0.250E−03 | 3.602 | 852 |
| 3237 | 352465 | Y | 0 | 0.00 | 0.00 | 3 | −4.41 | 0.20 | 69 | 0.27 | 2.92 | 4.675 | 17.850 | 0.250E−03 | 3.602 | 853 |
| 8022 | 352877 | Y | 69 | 0.27 | 2.92 | 3 | −4.41 | 0.20 | 0 | 0.00 | 0.00 | 4.675 | 17.850 | 0.260E−03 | 3.585 | 854 |
| 690 | 353811 | Y | 16 | −2.40 | 2.67 | 26 | 0.45 | 2.89 | 29 | 1.03 | 2.64 | 3.433 | 5.926 | 0.260E−03 | 3.585 | 855 |
| 8061 | 353017 | Y | 17 | −2.36 | 2.69 | 28 | 0.98 | 2.87 | 27 | 0.67 | 2.56 | 3.342 | 5.892 | 0.410E−03 | 3.387 | 856 |
| 1495 | 346019 | Y | 56 | 0.58 | 2.67 | 13 | −2.81 | 2.70 | 0 | 0.00 | 0.00 | 3.386 | 5.772 | 0.510E−03 | 3.292 | 857 |
| 7113 | 349728 | Y | 12 | −2.60 | 2.70 | 25 | 0.00 | 2.57 | 34 | 1.15 | 2.88 | 3.745 | 6.137 | 0.540E−03 | 3.268 | 858 |
| 7855 | 352224 | Y | 26 | −1.17 | 2.73 | 36 | 0.89 | 2.92 | 6 | 2.42 | 1.01 | 3.590 | 7.464 | 0.550E−03 | 3.260 | 859 |
| 1243 | 344998 | Y | 13 | −2.39 | 2.42 | 29 | 0.19 | 3.04 | 28 | 1.01 | 2.77 | 3.394 | 5.758 | 0.660E−03 | 3.180 | 860 |
| 2743 | 350667 | Y | 15 | 2.01 | 2.80 | 36 | 0.05 | 2.89 | 19 | −1.36 | 2.65 | 3.370 | 5.497 | 0.103E−02 | 2.987 | 861 |
| 621 | 353002 | Y | 35 | −0.08 | 2.90 | 29 | −0.59 | 2.89 | 8 | 3.16 | 2.18 | 3.755 | 6.133 | 0.150E−02 | 2.824 | 862 |
| 5844 | 345544 | Y | 12 | 2.52 | 2.55 | 36 | −0.22 | 3.09 | 24 | −0.71 | 2.53 | 3.229 | 5.399 | 0.216E−02 | 2.666 | 863 |

TABLE 20
Associations between DNA markers and retail beef yield sorted in decreasing order of
statistical significance. Locus is the ParAllele identifiers of the polymorphisms, N is
the number of genotypes, mean_0 is the mean retail beef yield for genotype 0, mean_1 is the
mean for genotype 1, and mean_2 is the mean of genotype 2, SD is the standard deviation, a
is the additive effect, k is the dominance effect, alpha is the average effect of allele
substitutions, tmax is the value of the t test, and log(1/P) is the P value determined from
100,000 permutation tests expressed as a positive integer. A minus value for a means the
mean of genotype 0 is lower than the mean of genotype 2, and a minus value for alpha means
that selecting for allele 0 will reduce the average values of retail beef yield in the
population.

| Locus | mean | SD | mean | SD | mean | SD | N | Freq | a | k | alpha | tmax | log(1/P) |
|---|---|---|---|---|---|---|---|---|---|---|---|---|---|
| 347657 | 0.60 | 2.98 | 0.91 | 2.21 | −3.49 | 2.01 | 72 | 0.69 | 2.04 | 1.15 | 2.93 | 8.435 | 5.0000 |
| 345212 | 1.39 | 2.22 | 0.20 | 3.04 | −2.86 | 2.35 | 72 | 0.59 | 2.12 | 0.44 | 2.29 | 7.776 | 5.0000 |
| 353110 | −2.68 | 2.52 | 1.78 | 1.98 | 0.31 | 2.94 | 71 | 0.27 | −1.50 | −1.98 | −2.83 | 7.787 | 4.5230 |
| 351736 | −2.78 | 2.71 | 0.17 | 2.58 | 1.25 | 2.68 | 72 | 0.38 | −2.01 | −0.46 | −2.25 | 6.957 | 4.5230 |
| 350185 | 0.58 | 2.81 | 0.07 | 2.77 | −5.29 | 0.82 | 72 | 0.77 | 2.94 | 0.83 | 4.25 | 12.540 | 4.3010 |
| 353986 | 0.51 | 3.01 | −1.08 | 2.78 | 2.96 | 1.07 | 72 | 0.72 | −1.23 | 2.30 | −2.44 | 8.348 | 3.8540 |
| 353512 | −1.80 | 2.87 | 0.85 | 2.58 | 1.73 | 2.49 | 72 | 0.58 | −1.77 | −0.50 | −1.63 | 6.306 | 3.7210 |
| 345055 | 0.00 | 0.00 | −4.41 | 0.20 | 0.27 | 2.92 | 72 | 0.02 | −0.14 | 33.67 | 4.22 | 17.850 | 3.6580 |
| 351887 | 0.53 | 2.96 | 1.42 | 2.48 | −2.30 | 2.32 | 72 | 0.68 | 1.42 | 1.63 | 2.25 | 6.490 | 3.6020 |
| 348851 | 1.06 | 2.85 | −0.02 | 2.57 | −2.93 | 2.56 | 72 | 0.67 | 2.00 | 0.46 | 2.31 | 6.637 | 3.6020 |
| 352465 | 0.00 | 0.00 | −4.41 | 0.20 | 0.27 | 2.92 | 72 | 0.02 | −0.14 | 33.67 | 4.22 | 17.850 | 3.6020 |
| 352877 | 0.27 | 2.92 | −4.41 | 0.20 | 0.00 | 0.00 | 72 | 0.98 | 0.14 | −33.67 | −4.22 | 17.850 | 3.5850 |
| 353811 | −2.40 | 2.67 | 0.45 | 2.89 | 1.03 | 2.64 | 71 | 0.41 | −1.71 | −0.66 | −1.92 | 5.926 | 3.5850 |
| 353017 | −2.36 | 2.69 | 0.98 | 2.87 | 0.67 | 2.56 | 72 | 0.43 | −1.51 | −1.20 | −1.77 | 5.892 | 3.3870 |
| 343676 | 2.65 | 0.75 | 0.97 | 2.51 | −1.07 | 3.19 | 71 | 0.30 | 1.86 | 0.10 | 1.79 | 8.286 | 3.3010 |
| 346019 | 0.58 | 2.67 | −2.81 | 2.70 | 0.00 | 0.00 | 69 | 0.91 | 0.29 | −10.69 | −2.23 | 5.772 | 3.2920 |
| 350492 | 0.58 | 2.95 | 1.14 | 2.59 | −2.54 | 2.29 | 70 | 0.69 | 1.56 | 1.36 | 2.38 | 6.259 | 3.2840 |
| 349728 | −2.60 | 2.70 | 0.00 | 2.57 | 1.15 | 2.88 | 71 | 0.35 | −1.88 | −0.39 | −2.10 | 6.137 | 3.2680 |
| 352224 | −1.17 | 2.73 | 0.89 | 2.92 | 2.42 | 1.01 | 68 | 0.65 | −1.79 | −0.15 | −1.72 | 7.464 | 3.2600 |
| 347013 | −2.51 | 2.90 | 1.43 | 2.82 | 0.07 | 2.65 | 72 | 0.33 | −1.29 | −2.05 | −2.21 | 6.147 | 3.2220 |
| 344998 | −2.39 | 2.42 | 0.19 | 3.04 | 1.01 | 2.77 | 70 | 0.39 | −1.70 | −0.52 | −1.89 | 5.758 | 3.1800 |
| 350295 | −1.09 | 2.86 | 0.49 | 3.11 | 2.11 | 1.52 | 71 | 0.65 | −1.60 | 0.01 | −1.61 | 6.131 | 3.0000 |
| 351886 | 0.62 | 2.84 | 1.33 | 2.82 | −2.54 | 2.29 | 71 | 0.73 | 1.58 | 1.45 | 2.61 | 5.925 | 2.9960 |
| 354319 | −1.55 | 2.54 | 1.42 | 3.16 | 0.67 | 2.31 | 72 | 0.57 | −1.11 | −1.68 | −0.85 | 5.384 | 2.9910 |
| 350667 | 2.01 | 2.80 | 0.05 | 2.89 | −1.36 | 2.65 | 70 | 0.47 | 1.69 | −0.16 | 1.70 | 5.497 | 2.9870 |
| 343932 | −2.19 | 2.38 | 0.76 | 3.52 | 1.02 | 2.24 | 70 | 0.42 | −1.60 | −0.84 | −1.82 | 5.478 | 2.9630 |
| 344277 | 1.65 | 2.50 | 0.46 | 3.11 | −1.60 | 2.38 | 72 | 0.44 | 1.62 | 0.27 | 1.58 | 5.613 | 2.9550 |
| 343738 | 0.74 | 2.80 | −0.70 | 2.96 | −4.10 | 1.14 | 72 | 0.81 | 2.42 | 0.40 | 3.02 | 8.710 | 2.9510 |
| 343686 | 0.54 | 3.06 | 1.37 | 2.58 | −2.22 | 2.47 | 65 | 0.68 | 1.38 | 1.60 | 2.20 | 5.689 | 2.9240 |
| 348679 | −2.20 | 2.17 | 0.07 | 2.82 | 1.18 | 3.21 | 70 | 0.42 | −1.69 | −0.34 | −1.78 | 5.659 | 2.9100 |
| 351219 | 1.51 | 2.84 | −0.06 | 2.48 | −1.41 | 2.76 | 70 | 0.51 | 1.46 | −0.08 | 1.46 | 5.227 | 2.9070 |
| 353161 | 1.03 | 2.64 | 0.45 | 2.95 | −1.99 | 2.78 | 72 | 0.58 | 1.51 | 0.62 | 1.65 | 5.186 | 2.8390 |
| 350570 | −1.01 | 2.92 | 0.14 | 2.82 | 2.25 | 2.63 | 69 | 0.61 | −1.63 | 0.29 | −1.73 | 5.404 | 2.8270 |
| 353002 | −0.08 | 2.90 | −0.59 | 2.89 | 3.16 | 2.18 | 72 | 0.69 | −1.62 | 1.31 | −2.42 | 6.133 | 2.8240 |
| 343925 | 0.36 | 2.30 | −1.44 | 2.97 | 1.81 | 3.27 | 70 | 0.57 | −0.73 | 3.48 | −1.09 | 5.263 | 2.8210 |
| 351957 | 1.47 | 2.65 | 0.08 | 3.20 | −1.57 | 2.26 | 71 | 0.53 | 1.52 | 0.09 | 1.53 | 5.369 | 2.8100 |
| 345649 | 0.31 | 2.82 | 1.01 | 2.87 | −2.56 | 2.67 | 72 | 0.70 | 1.44 | 1.49 | 2.29 | 5.554 | 2.8010 |
| 348825 | 0.00 | 0.00 | 1.62 | 2.89 | −0.80 | 2.79 | 71 | 0.18 | 0.40 | 5.05 | −0.88 | 4.745 | 2.7990 |
| 345814 | 2.33 | 2.39 | −0.31 | 2.71 | −0.68 | 3.05 | 72 | 0.38 | 1.51 | −0.75 | 1.77 | 5.311 | 2.7540 |
| 346220 | 0.00 | 0.00 | 2.31 | 0.99 | −0.30 | 2.93 | 68 | 0.06 | 0.15 | 16.40 | −2.02 | 6.682 | 2.7120 |
| 353802 | 0.36 | 2.94 | −1.27 | 3.10 | 2.06 | 1.53 | 72 | 0.74 | −0.85 | 2.92 | −2.06 | 5.928 | 2.6970 |
| 345183 | 0.62 | 2.84 | 1.02 | 2.90 | −2.54 | 2.29 | 72 | 0.72 | 1.58 | 1.25 | 2.46 | 5.504 | 2.6970 |
| 347846 | 1.55 | 2.79 | −0.04 | 2.86 | −1.28 | 2.46 | 70 | 0.55 | 1.42 | −0.12 | 1.40 | 5.052 | 2.6700 |
| 349243 | −2.03 | 2.68 | −0.09 | 3.10 | 1.19 | 2.56 | 72 | 0.39 | −1.61 | −0.20 | −1.68 | 5.335 | 2.6680 |
| 348659 | −1.47 | 2.60 | 0.58 | 3.48 | 1.25 | 1.81 | 72 | 0.53 | −1.36 | −0.51 | −1.31 | 5.056 | 2.6660 |
| 345544 | 2.52 | 2.55 | −0.22 | 3.09 | −0.71 | 2.53 | 72 | 0.42 | 1.61 | −0.70 | 1.80 | 5.399 | 2.6660 |
| 352969 | −1.65 | 3.26 | 0.18 | 2.84 | 1.35 | 2.36 | 72 | 0.47 | −1.50 | −0.22 | −1.52 | 5.020 | 2.6640 |
| 352779 | 0.67 | 2.88 | 0.27 | 2.62 | −3.40 | 2.78 | 72 | 0.71 | 2.04 | 0.80 | 2.72 | 5.866 | 2.6400 |
| 349785 | 0.01 | 2.70 | −1.23 | 3.08 | 1.60 | 2.55 | 71 | 0.50 | −0.80 | 2.56 | −0.80 | 4.974 | 2.6250 |
| 342775 | 1.67 | 1.64 | −1.10 | 2.42 | 0.15 | 3.68 | 69 | 0.38 | 0.76 | −2.64 | 1.23 | 5.142 | 2.6220 |
| 352028 | −3.80 | 1.96 | 0.21 | 1.86 | 0.44 | 3.31 | 72 | 0.23 | −2.12 | −0.89 | −3.14 | 6.788 | 2.6180 |
| 352133 | −0.01 | 3.19 | 1.17 | 2.45 | −2.32 | 2.37 | 71 | 0.71 | 1.16 | 2.02 | 2.14 | 5.665 | 2.6020 |
| 348096 | 0.79 | 2.91 | 0.36 | 2.63 | −2.36 | 2.60 | 72 | 0.69 | 1.57 | 0.73 | 2.00 | 5.201 | 2.6020 |
| 354493 | 1.44 | 2.80 | −1.74 | 1.63 | 0.53 | 2.91 | 57 | 0.35 | 0.45 | −5.99 | 1.27 | 5.349 | 2.5870 |
| 348824 | −0.72 | 2.77 | 1.73 | 2.95 | 0.00 | 0.00 | 70 | 0.84 | −0.36 | −5.81 | 1.04 | 4.570 | 2.5780 |
| 348555 | 4.90 | 2.48 | −0.23 | 3.14 | −0.37 | 2.65 | 68 | 0.21 | 2.64 | −0.95 | 4.07 | 6.612 | 2.5470 |
| 349040 | 0.78 | 2.92 | −1.44 | 2.68 | 0.00 | 0.00 | 69 | 0.80 | 0.39 | −4.69 | −0.72 | 4.410 | 2.5330 |
| 349163 | 2.41 | 0.95 | 0.34 | 3.85 | −0.71 | 2.53 | 70 | 0.29 | 1.56 | −0.33 | 1.78 | 5.566 | 2.5320 |
| 343107 | 0.61 | 2.75 | 0.74 | 3.03 | −2.18 | 2.52 | 72 | 0.61 | 1.40 | 1.09 | 1.73 | 5.005 | 2.5270 |
| 353686 | −0.36 | 2.42 | 3.58 | 2.63 | −0.38 | 2.70 | 63 | 0.23 | 0.01 | 395.00 | −2.12 | 5.552 | 2.5260 |
| 346831 | −1.02 | 2.86 | −0.26 | 2.93 | 1.76 | 2.62 | 72 | 0.53 | −1.39 | 0.45 | −1.43 | 4.847 | 2.5170 |
| 344847 | 2.42 | 1.63 | −2.11 | 2.28 | 0.38 | 3.00 | 72 | 0.14 | 1.02 | −3.44 | 3.56 | 7.008 | 2.5030 |
| 344018 | −2.65 | 2.50 | −0.55 | 3.35 | 1.21 | 2.22 | 70 | 0.27 | −1.93 | −0.09 | −2.01 | 5.627 | 2.4950 |
| 348894 | 0.17 | 2.53 | −1.41 | 3.41 | 1.62 | 2.62 | 72 | 0.60 | −0.73 | 3.18 | −1.21 | 4.942 | 2.4840 |
| 351901 | 1.26 | 2.38 | 0.18 | 2.84 | −1.65 | 3.26 | 71 | 0.52 | 1.46 | 0.26 | 1.47 | 4.831 | 2.4740 |
| 351684 | −0.07 | 3.03 | 2.94 | 0.26 | 0.00 | 0.00 | 71 | 0.98 | −0.04 | −85.00 | 2.81 | 10.779 | 2.4660 |
| 348976 | −1.19 | 2.22 | 0.19 | 3.31 | 1.66 | 2.58 | 72 | 0.55 | −1.42 | 0.03 | −1.43 | 4.978 | 2.4650 |
| 349018 | 1.84 | 2.74 | −0.58 | 3.33 | −0.85 | 2.11 | 72 | 0.49 | 1.34 | −0.80 | 1.36 | 4.804 | 2.4620 |
| 350070 | −1.10 | 2.07 | −2.32 | 2.60 | 1.22 | 2.72 | 70 | 0.23 | −1.16 | 2.05 | 0.13 | 5.716 | 2.4530 |
| 352396 | 2.41 | 2.18 | 0.02 | 2.82 | −0.59 | 3.06 | 72 | 0.32 | 1.50 | −0.59 | 1.82 | 5.145 | 2.4450 |
| 347844 | 0.70 | 2.92 | −1.65 | 2.67 | 0.00 | 0.00 | 72 | 0.85 | 0.35 | −5.71 | −1.04 | 4.429 | 2.4320 |
| 346426 | −0.32 | 2.93 | −0.58 | 2.72 | 2.84 | 2.65 | 71 | 0.69 | −1.58 | 1.16 | −2.28 | 5.314 | 2.4290 |
| 346881 | 2.41 | 2.18 | 0.02 | 2.82 | −0.59 | 3.06 | 72 | 0.32 | 1.50 | −0.59 | 1.82 | 5.145 | 2.4290 |
| 351830 | −0.59 | 3.06 | 0.02 | 2.82 | 2.41 | 2.18 | 72 | 0.68 | −1.50 | 0.59 | −1.82 | 5.145 | 2.4150 |
| 348549 | −1.22 | 2.78 | 0.10 | 2.92 | 1.97 | 2.90 | 70 | 0.56 | −1.59 | 0.17 | −1.63 | 4.957 | 2.4100 |
| 351744 | 0.00 | 0.00 | −2.03 | 2.52 | 0.57 | 2.97 | 72 | 0.12 | −0.28 | 8.12 | 1.48 | 4.597 | 2.4060 |
| 345624 | −2.57 | 1.89 | −0.03 | 3.04 | 0.66 | 2.93 | 72 | 0.28 | −1.61 | −0.57 | −2.03 | 5.477 | 2.4060 |
| 352026 | −1.20 | 2.14 | −0.01 | 3.40 | 1.43 | 2.46 | 72 | 0.49 | −1.31 | 0.10 | −1.31 | 4.802 | 2.4010 |
| 342505 | −0.60 | 2.98 | 1.56 | 2.64 | 0.00 | 0.00 | 72 | 0.83 | −0.30 | −6.20 | 0.94 | 4.336 | 2.3970 |
| 344701 | −0.06 | 3.00 | 0.00 | 0.00 | 3.17 | 0.43 | 72 | 0.96 | −1.61 | 0.96 | −3.04 | 10.401 | 2.3890 |
| 353130 | 0.27 | 2.56 | −1.37 | 3.26 | 1.33 | 2.63 | 72 | 0.50 | −0.53 | 4.09 | −0.53 | 4.672 | 2.3890 |
| 348115 | −1.20 | 2.14 | −0.01 | 3.40 | 1.43 | 2.46 | 72 | 0.49 | −1.31 | 0.10 | −1.31 | 4.802 | 2.3840 |
| 346942 | −1.22 | 2.99 | −0.26 | 2.61 | 1.49 | 2.83 | 71 | 0.49 | −1.35 | 0.29 | −1.35 | 4.677 | 2.3560 |
| 354094 | 0.19 | 3.08 | −1.30 | 2.99 | 2.19 | 1.31 | 69 | 0.81 | −1.00 | 2.49 | −2.55 | 5.732 | 2.3560 |
| 344838 | 1.36 | 2.34 | 0.29 | 2.93 | −1.59 | 3.14 | 72 | 0.49 | 1.48 | 0.27 | 1.47 | 4.880 | 2.3560 |
| 344830 | 1.37 | 2.51 | −1.55 | 3.08 | −0.20 | 2.82 | 65 | 0.57 | 0.79 | −2.72 | 0.49 | 4.725 | 2.3330 |
| 342541 | 1.45 | 1.70 | 0.47 | 3.28 | −1.15 | 2.83 | 70 | 0.41 | 1.30 | 0.25 | 1.24 | 4.775 | 2.3330 |
| 343029 | 0.10 | 2.48 | 1.75 | 3.02 | −1.07 | 3.05 | 72 | 0.50 | 0.59 | 3.82 | 0.59 | 4.682 | 2.3280 |
| 352311 | 0.00 | 0.00 | 3.32 | 0.40 | 0.14 | 3.08 | 63 | 0.02 | −0.07 | −46.43 | −3.17 | 9.781 | 2.3210 |
| 353532 | −1.70 | 2.97 | −0.06 | 2.93 | 1.16 | 2.66 | 66 | 0.45 | −1.43 | −0.15 | −1.45 | 4.638 | 2.3070 |
| 344436 | −1.46 | 2.68 | 0.82 | 2.91 | 1.31 | 2.77 | 72 | 0.58 | −1.39 | −0.65 | −1.25 | 4.721 | 2.2970 |
| 351850 | 0.50 | 3.03 | 0.00 | 0.00 | −1.62 | 2.24 | 66 | 0.74 | 1.06 | 0.53 | 1.33 | 4.300 | 2.2950 |
| 346624 | 0.00 | 0.00 | 2.26 | 1.98 | −0.62 | 2.98 | 54 | 0.09 | 0.31 | 8.29 | −1.78 | 4.904 | 2.2840 |
| 352566 | 0.07 | 3.09 | −1.07 | 2.99 | 2.93 | 1.87 | 66 | 0.82 | −1.43 | 1.80 | −3.07 | 5.610 | 2.2790 |
| 344973 | 0.59 | 2.55 | −1.58 | 2.87 | 0.00 | 0.00 | 64 | 0.84 | 0.29 | −6.36 | −0.96 | 4.168 | 2.2720 |
| 350461 | −1.12 | 3.08 | −0.06 | 2.48 | 1.51 | 2.84 | 71 | 0.50 | −1.31 | 0.19 | −1.31 | 4.552 | 2.2650 |
| 344191 | 0.74 | 3.16 | −0.43 | 2.53 | −2.43 | 1.84 | 72 | 0.75 | 1.58 | 0.26 | 1.79 | 5.469 | 2.2640 |
| 344183 | 0.00 | 0.00 | 2.03 | 1.50 | −0.14 | 3.10 | 72 | 0.09 | 0.07 | 30.00 | −1.65 | 4.810 | 2.2600 |
| 344536 | 1.40 | 2.70 | −1.25 | 2.97 | 0.29 | 2.75 | 72 | 0.54 | 0.55 | −3.77 | 0.38 | 4.590 | 2.2540 |
| 344864 | −0.20 | 2.39 | 1.34 | 3.28 | −1.65 | 2.92 | 72 | 0.62 | 0.72 | 3.12 | 1.29 | 4.719 | 2.2490 |
| 350429 | 0.97 | 3.08 | −0.45 | 2.74 | −2.36 | 2.03 | 72 | 0.70 | 1.67 | 0.15 | 1.76 | 5.476 | 2.2370 |
| 346717 | 0.00 | 0.00 | −1.25 | 2.99 | 0.88 | 2.69 | 72 | 0.19 | −0.44 | 3.84 | 0.62 | 4.089 | 2.2360 |
| 350280 | 0.09 | 2.79 | 1.02 | 2.91 | −2.09 | 2.92 | 72 | 0.65 | 1.09 | 1.85 | 1.68 | 4.799 | 2.2350 |
| 348797 | 1.78 | 2.38 | −0.05 | 2.61 | −0.91 | 3.29 | 70 | 0.44 | 1.34 | −0.36 | 1.40 | 4.630 | 2.2350 |
| 346006 | 0.00 | 0.00 | 3.20 | 0.55 | −0.06 | 3.02 | 71 | 0.02 | 0.03 | 107.67 | −3.06 | 9.558 | 2.2270 |
| 354108 | 0.00 | 0.00 | 3.20 | 0.55 | −0.06 | 3.00 | 72 | 0.02 | 0.03 | 107.67 | −3.07 | 9.632 | 2.2260 |
| 346122 | −0.01 | 2.99 | 3.20 | 0.55 | 0.00 | 0.00 | 70 | 0.98 | −0.01 | −641.00 | 3.06 | 9.410 | 2.2210 |
| 351288 | 1.26 | 2.95 | 0.57 | 2.84 | −1.49 | 2.75 | 71 | 0.46 | 1.38 | 0.50 | 1.32 | 4.580 | 2.2180 |
| 344010 | 0.29 | 2.93 | −1.77 | 2.67 | 3.06 | 2.06 | 72 | 0.85 | −1.39 | 2.49 | −3.83 | 6.295 | 2.2180 |
| 353682 | −1.14 | 2.22 | 0.28 | 3.71 | 1.31 | 1.77 | 71 | 0.56 | −1.23 | −0.16 | −1.20 | 4.615 | 2.2160 |
| 349024 | 0.94 | 2.78 | −0.09 | 3.07 | −1.97 | 2.61 | 72 | 0.66 | 1.46 | 0.29 | 1.59 | 4.715 | 2.2150 |
| 352077 | 3.13 | 1.75 | 0.26 | 1.57 | −0.20 | 3.24 | 71 | 0.17 | 1.67 | −0.72 | 2.46 | 5.802 | 2.2080 |
| 351296 | 0.64 | 2.86 | −2.04 | 3.04 | 0.00 | 0.00 | 70 | 0.86 | 0.32 | −7.37 | −1.40 | 4.302 | 2.2080 |
| 345504 | −1.35 | 2.96 | 0.37 | 2.97 | 1.44 | 2.60 | 70 | 0.54 | −1.40 | −0.23 | −1.37 | 4.626 | 2.2010 |
| 351514 | −2.71 | 3.19 | 0.66 | 2.64 | 0.45 | 2.84 | 71 | 0.36 | −1.58 | −1.13 | −2.08 | 4.882 | 2.2000 |
| 349752 | 0.70 | 2.49 | −0.37 | 3.12 | −3.43 | 2.57 | 66 | 0.72 | 2.06 | 0.48 | 2.50 | 5.532 | 2.2000 |
| 349919 | −0.06 | 3.00 | 3.20 | 0.55 | 0.00 | 0.00 | 72 | 0.98 | −0.03 | −107.67 | 3.07 | 9.632 | 2.1990 |
| 351525 | 2.93 | 1.79 | −0.86 | 3.19 | 0.61 | 2.73 | 66 | 0.32 | 1.16 | −2.27 | 2.12 | 5.956 | 2.1910 |
| 346742 | −0.75 | 3.20 | 0.14 | 2.88 | 1.97 | 1.83 | 70 | 0.61 | −1.36 | 0.35 | −1.46 | 4.840 | 2.1860 |
| 345736 | 1.65 | 2.05 | −0.03 | 3.37 | −0.87 | 2.65 | 72 | 0.45 | 1.26 | −0.33 | 1.30 | 4.571 | 2.1820 |
| 354432 | 0.87 | 2.83 | 0.03 | 2.90 | −2.08 | 2.75 | 72 | 0.67 | 1.48 | 0.43 | 1.70 | 4.693 | 2.1760 |
| 344131 | 1.02 | 2.32 | −2.40 | 2.29 | 0.10 | 3.19 | 70 | 0.42 | 0.46 | −6.43 | 0.93 | 5.336 | 2.1720 |
| 350495 | −0.48 | 2.78 | 0.09 | 3.34 | 2.87 | 1.70 | 69 | 0.72 | −1.68 | 0.66 | −2.17 | 5.486 | 2.1690 |
| 348287 | 0.28 | 2.90 | −4.62 | 1.15 | 0.00 | 0.00 | 72 | 0.98 | 0.14 | −34.00 | −4.42 | 9.212 | 2.1620 |
| 349907 | −1.97 | 2.61 | 0.00 | 3.11 | 1.00 | 2.77 | 70 | 0.33 | −1.48 | −0.33 | −1.65 | 4.644 | 2.1610 |
| 353531 | −1.64 | 2.70 | 0.45 | 2.95 | 1.03 | 2.64 | 70 | 0.41 | −1.33 | −0.57 | −1.48 | 4.529 | 2.1600 |
| 354336 | 0.53 | 3.10 | −1.40 | 2.30 | 3.71 | 1.65 | 69 | 0.81 | −1.59 | 2.21 | −3.78 | 7.550 | 2.1580 |
| 346884 | −1.93 | 2.47 | 0.52 | 2.77 | 0.71 | 3.15 | 71 | 0.42 | −1.32 | −0.86 | −1.51 | 4.537 | 2.1560 |
| 349636 | −1.41 | 2.86 | 1.53 | 1.94 | −0.09 | 3.20 | 72 | 0.28 | −0.66 | −3.45 | −1.64 | 4.755 | 2.1530 |
| 347279 | −0.09 | 3.11 | 0.82 | 2.83 | −2.35 | 1.15 | 70 | 0.79 | 1.13 | 1.81 | 2.30 | 5.852 | 2.1520 |
| 348339 | 0.84 | 2.73 | 0.07 | 3.23 | −2.21 | 2.52 | 72 | 0.69 | 1.52 | 0.50 | 1.82 | 4.667 | 2.1400 |
| 353853 | 1.70 | 2.63 | −0.50 | 2.84 | −1.01 | 3.03 | 71 | 0.55 | 1.35 | −0.62 | 1.27 | 4.496 | 2.1400 |
| 346382 | −3.30 | 2.20 | −0.21 | 2.88 | 0.59 | 2.91 | 71 | 0.18 | −1.94 | −0.59 | −2.67 | 5.376 | 2.1310 |
| 352445 | −2.04 | 2.86 | 0.39 | 2.73 | 0.82 | 3.00 | 72 | 0.42 | −1.43 | −0.70 | −1.60 | 4.596 | 2.1290 |
| 345165 | 0.84 | 2.93 | 0.14 | 2.99 | −2.06 | 2.61 | 70 | 0.61 | 1.45 | 0.52 | 1.61 | 4.642 | 2.1270 |
| 345555 | 0.05 | 2.65 | −0.65 | 3.34 | 2.40 | 2.21 | 72 | 0.68 | −1.18 | 1.60 | −1.85 | 4.941 | 2.1270 |
| 350397 | −0.03 | 2.87 | 2.43 | 2.92 | −0.75 | 2.73 | 69 | 0.62 | 0.36 | 7.83 | 1.01 | 4.730 | 2.1260 |
| 348558 | −2.50 | 2.41 | −0.29 | 2.91 | 0.85 | 2.96 | 70 | 0.28 | −1.68 | −0.32 | −1.91 | 5.047 | 2.1240 |
| 354061 | −0.69 | 3.25 | 0.02 | 3.03 | 1.87 | 1.70 | 71 | 0.58 | −1.28 | 0.45 | −1.38 | 4.610 | 2.1210 |
| 351203 | 0.69 | 2.94 | −1.45 | 2.38 | 0.00 | 0.00 | 70 | 0.88 | 0.34 | −5.20 | −1.01 | 4.121 | 2.1120 |
| 348607 | 2.82 | 2.44 | 0.52 | 2.84 | −0.75 | 2.94 | 71 | 0.30 | 1.78 | −0.29 | 2.00 | 5.261 | 2.1120 |
| 352618 | 0.97 | 3.02 | 0.66 | 2.77 | −1.68 | 2.83 | 68 | 0.52 | 1.32 | 0.77 | 1.37 | 4.395 | 2.1120 |
| 353471 | −0.23 | 3.09 | 1.79 | 1.75 | 0.00 | 0.00 | 72 | 0.92 | −0.12 | −16.57 | 1.50 | 4.343 | 2.1080 |
| 347554 | −0.14 | 2.77 | −0.90 | 3.34 | 1.74 | 2.07 | 65 | 0.55 | −0.94 | 1.81 | −1.12 | 4.508 | 2.1080 |
| 348079 | 0.25 | 2.81 | 0.57 | 3.01 | −3.16 | 2.65 | 67 | 0.77 | 1.71 | 1.19 | 2.79 | 5.023 | 2.1050 |
| 348387 | 0.00 | 0.00 | −1.11 | 3.35 | 0.95 | 2.48 | 72 | 0.22 | −0.47 | 3.34 | 0.43 | 3.906 | 2.1020 |
| 351975 | −1.95 | 2.65 | 0.70 | 2.97 | 0.60 | 2.82 | 72 | 0.42 | −1.27 | −1.08 | −1.49 | 4.496 | 2.1010 |
| 352556 | −1.86 | 2.21 | 0.00 | 0.00 | 0.71 | 3.11 | 57 | 0.19 | −1.29 | −0.45 | −1.64 | 4.320 | 2.1000 |
| 348168 | −0.51 | 2.99 | −0.23 | 2.68 | 2.57 | 2.87 | 72 | 0.65 | −1.54 | 0.82 | −1.91 | 4.715 | 2.0990 |
| 349641 | 0.00 | 0.00 | 1.85 | 0.90 | −0.09 | 3.08 | 72 | 0.04 | 0.04 | 42.11 | −1.69 | 5.184 | 2.0940 |
| 354141 | 2.23 | 3.18 | 0.59 | 2.55 | −1.14 | 2.93 | 72 | 0.35 | 1.69 | 0.03 | 1.67 | 4.776 | 2.0890 |
| 346864 | −1.03 | 2.44 | 0.49 | 3.39 | 1.61 | 1.96 | 72 | 0.62 | −1.32 | −0.15 | −1.27 | 4.732 | 2.0750 |
| 350780 | 2.23 | 3.18 | 0.59 | 2.55 | −1.14 | 2.93 | 72 | 0.35 | 1.69 | 0.03 | 1.67 | 4.776 | 2.0700 |
| 353286 | −2.72 | 2.74 | 0.29 | 3.31 | 0.62 | 2.46 | 72 | 0.32 | −1.67 | −0.80 | −2.15 | 4.894 | 2.0690 |
| 348641 | 0.00 | 0.00 | −2.21 | 2.70 | 0.50 | 2.92 | 72 | 0.09 | −0.25 | 9.84 | 1.77 | 4.266 | 2.0680 |
| 349178 | −0.34 | 3.04 | −0.24 | 2.67 | 2.74 | 2.38 | 72 | 0.74 | −1.54 | 0.94 | −2.22 | 4.867 | 2.0670 |
| 348452 | 1.06 | 2.75 | −0.41 | 3.01 | −1.89 | 2.76 | 72 | 0.67 | 1.48 | 0.00 | 1.48 | 4.616 | 2.0660 |
| 348122 | 1.86 | 3.30 | 0.29 | 2.65 | −0.98 | 2.82 | 69 | 0.40 | 1.42 | −0.11 | 1.45 | 4.392 | 2.0610 |
| 350668 | −1.22 | 2.66 | 0.14 | 2.90 | 1.65 | 3.12 | 72 | 0.53 | −1.44 | 0.05 | −1.44 | 4.484 | 2.0540 |
| 353733 | 0.87 | 2.62 | −0.57 | 3.52 | −2.29 | 2.36 | 70 | 0.71 | 1.58 | 0.09 | 1.64 | 4.618 | 2.0520 |
| 354047 | −1.77 | 3.36 | 1.08 | 2.69 | −0.03 | 2.71 | 71 | 0.44 | −0.87 | −2.28 | −1.09 | 4.426 | 2.0520 |
| 351572 | 0.81 | 2.79 | 0.46 | 2.97 | −1.92 | 2.66 | 71 | 0.62 | 1.36 | 0.74 | 1.61 | 4.510 | 2.0510 |
| 354312 | 0.52 | 2.81 | −2.39 | 3.00 | 0.00 | 0.00 | 72 | 0.92 | 0.26 | −10.19 | −1.99 | 4.216 | 2.0510 |
| 348605 | −0.84 | 2.95 | 0.13 | 3.29 | 1.60 | 1.81 | 72 | 0.58 | −1.22 | 0.20 | −1.26 | 4.447 | 2.0510 |
| 345669 | −2.47 | 1.84 | −3.67 | 2.14 | 0.67 | 2.77 | 72 | 0.10 | −1.57 | 1.76 | 0.62 | 5.884 | 2.0510 |
| 343565 | 2.40 | 3.41 | 0.49 | 2.37 | −1.11 | 3.04 | 71 | 0.37 | 1.75 | −0.09 | 1.80 | 4.718 | 2.0490 |
| 351369 | 0.88 | 3.00 | 0.14 | 2.69 | −2.10 | 2.96 | 72 | 0.62 | 1.49 | 0.50 | 1.68 | 4.587 | 2.0480 |
| 346109 | 0.42 | 3.13 | 1.44 | 2.75 | −1.14 | 2.66 | 72 | 0.51 | 0.78 | 2.31 | 0.80 | 4.364 | 2.0440 |
| 349457 | 1.02 | 3.01 | 0.26 | 3.06 | −1.77 | 2.15 | 71 | 0.51 | 1.40 | 0.46 | 1.41 | 4.557 | 2.0410 |
| 351094 | −0.42 | 2.97 | 1.67 | 2.61 | 0.00 | 0.00 | 72 | 0.88 | −0.21 | −8.95 | 1.23 | 3.950 | 2.0380 |
| 354465 | −4.13 | 2.46 | −1.22 | 2.00 | 0.65 | 2.93 | 72 | 0.14 | −2.39 | −0.22 | −2.77 | 5.847 | 2.0330 |
| 349414 | 0.71 | 2.64 | −0.21 | 3.39 | −2.47 | 1.67 | 72 | 0.72 | 1.59 | 0.42 | 1.88 | 5.369 | 2.0320 |
| 352554 | 0.99 | 2.86 | 0.82 | 2.91 | −1.68 | 2.82 | 67 | 0.54 | 1.33 | 0.87 | 1.42 | 4.342 | 2.0310 |
| 353866 | 0.00 | 0.00 | −1.19 | 3.05 | 0.88 | 2.69 | 71 | 0.18 | −0.44 | 3.70 | 0.59 | 3.870 | 2.0310 |
| 352891 | 0.99 | 2.85 | 0.32 | 2.89 | −1.59 | 2.84 | 72 | 0.56 | 1.29 | 0.48 | 1.37 | 4.348 | 2.0300 |
| 351095 | 0.51 | 2.62 | 0.55 | 3.21 | −2.20 | 2.49 | 72 | 0.62 | 1.35 | 1.03 | 1.68 | 4.564 | 2.0210 |
| 345058 | 1.97 | 3.21 | 0.29 | 2.92 | −1.25 | 2.55 | 72 | 0.42 | 1.61 | −0.04 | 1.62 | 4.618 | 2.0190 |
| 344746 | −4.02 | 1.93 | 0.08 | 2.94 | 0.39 | 2.90 | 72 | 0.17 | −2.20 | −0.86 | −3.44 | 6.044 | 2.0190 |
| 343069 | −0.72 | 2.87 | 1.42 | 2.95 | −2.88 | 1.23 | 62 | 0.73 | 1.08 | 2.98 | 2.59 | 7.162 | 2.0190 |
| 354167 | 1.59 | 2.86 | −0.80 | 2.82 | −0.89 | 2.71 | 72 | 0.56 | 1.24 | −0.93 | 1.11 | 4.314 | 2.0110 |
| 344411 | 0.26 | 2.96 | 1.74 | 2.98 | −2.09 | 2.65 | 64 | 0.79 | 1.17 | 2.26 | 2.71 | 4.843 | 2.0070 |
| 346373 | 1.54 | 3.14 | −0.15 | 2.39 | −1.02 | 3.09 | 71 | 0.47 | 1.28 | −0.32 | 1.30 | 4.271 | 2.0030 |

TABLE 21

| | | | | | | | | | | | | | | SEQ |
|---|---|---|---|---|---|---|---|---|---|---|---|---|---|---|
| RBY rank | ident | exp | log(1/P) | | rerank | exp | log(1/P) | | rank | exp | log(1/P) | rank | comb_log(1/P) | ID NO: |
| 1 | 347657 | RBY | 5.000 | 1 | P8F | 0.973 | 1025 | CWT | 0.049 | 7835 | 3.504 | | | 843 |
| 2 | 345212 | RBY | 5.000 | 2 | P8F | 0.103 | 6923 | CWT | 0.319 | 4531 | 3.404 | | | 844 |
| 3 | 351736 | RBY | 4.523 | 4 | P8F | 0.444 | 3258 | CWT | 0.750 | 1889 | 3.214 | | | 846 |
| 4 | 353110 | RBY | 4.523 | 3 | P8F | 0.511 | 2805 | CWT | 0.279 | 4954 | 3.147 | | | 845 |
| 5 | 350185 | RBY | 4.301 | 5 | P8F | 0.768 | 1628 | CWT | 0.543 | 2895 | 3.086 | | | 847 |
| 6 | 345055 | RBY | 3.658 | 8 | P8F | 1.037 | 878 | CWT | 2.519 | 43 | 3.031 | | | 850 |
| 7 | 352465 | RBY | 3.602 | 11 | P8F | 1.036 | 879 | CWT | 2.538 | 39 | 2.997 | | | 853 |
| 8 | 352877 | RBY | 3.585 | 12 | P8F | 1.040 | 869 | CWT | 2.527 | 42 | 2.985 | | | 854 |
| 9 | 353512 | RBY | 3.721 | 7 | P8F | 1.079 | 800 | CWT | 0.836 | 1574 | 2.800 | | | 849 |
| 10 | 353986 | RBY | 3.854 | 6 | P8F | 0.614 | 2264 | CWT | 0.101 | 7086 | 2.688 | | | 848 |
| 11 | 344998 | RBY | 3.180 | 21 | P8F | 2.290 | 56 | CWT | 0.899 | 1385 | 2.651 | | | 860 |
| 12 | 353002 | RBY | 2.824 | 34 | P8F | 1.452 | 385 | CWT | 2.928 | 21 | 2.613 | | | 862 |
| 13 | 350667 | RBY | 2.987 | 25 | P8F | 1.338 | 474 | CWT | 2.277 | 84 | 2.594 | | | 861 |
| 14 | 348851 | RBY | 3.602 | 10 | P8F | 0.054 | 7714 | CWT | 0.991 | 1135 | 2.575 | | | 852 |
| 15 | 353811 | RBY | 3.585 | 13 | P8F | 0.774 | 1614 | CWT | 0.276 | 4977 | 2.565 | | | 855 |
| 16 | 351887 | RBY | 3.602 | 9 | P8F | 0.035 | 8045 | CWT | 0.930 | 1305 | 2.562 | | | 851 |
| 17 | 353017 | RBY | 3.387 | 14 | P8F | 1.306 | 503 | CWT | 0.392 | 3906 | 2.541 | | | 856 |
| 18 | 352224 | RBY | 3.260 | 19 | P8F | 0.872 | 1284 | CWT | 0.979 | 1169 | 2.482 | | | 859 |
| 19 | 349728 | RBY | 3.268 | 18 | P8F | 0.036 | 8029 | CWT | 1.656 | 295 | 2.461 | | | 858 |
| 20 | 346019 | RBY | 3.292 | 16 | P8F | 0.922 | 1146 | CWT | 0.587 | 2648 | 2.446 | | | 857 |
| 21 | 345544 | RBY | 2.666 | 46 | P8F | 2.783 | 17 | CWT | 1.054 | 989 | 2.417 | | | 863 |

The best 21 SNP for retail beef yield after analyses of P8 fat and carcass weight are considered.
RBY rank and ident refers to Table 20, exp is experiment or trait, rby is for retail beef yield, p8f for p8fat, cwt for carcass weight, rerank is the relative ranking of the retail beef yield SNP from Table 19 after consideration of the carcass weight and p8 fat thickness, and comb_log(1/P) is the combined significance value of the three experiments.

This shows that the top 5 associations are the same as for retail beef yield purely, but that some of the associations further down are reranked and given greater importance based on fat thickness and carcass weight. The results may be correlated by way of the sequence identification number (second column in each Table).

TABLE 22
SNP with high probability of association to RBY after consideration of P8
fat and carcass weight.

| Ident | scaffold | contig chr. | Comment | | SEQ ID NO: |
|---|---|---|---|---|---|
| 352465 | SCAFFOLD80596_16424 | contig65286 | Hsa3 | SENP7 exon | 853 |
| 352877 | SCAFFOLD30708_2335 | contig186691 | Hsa3 | IMPG2 intron | 854 |
| 344998 | SCAFFOLD282025_3342 | contig166161 | Hsa16 | EFCBP2 exon | 855 |
| 353002 | IBISS4snp640 | | Bta24 | DSC2 | 856 |
| 350667 | SCAFFOLD121139_3191 | contig52631 | Hsa12 | intergenic SDS3-LOC144747 | 857 |
| 353811 | SCAFFOLD195717_2202 | contig217113 | no chr. | | 858 |
| 353017 | IBISS4snp879 | | Hsa5 | C5orf18, region incl DCP2, XBPP1 | 859 |
| 352224 | SCAFFOLD240110_25059 | contig482987 | Hsa18 | LAMA3 intron | 860 |
| 349728 | SCAFFOLD91511_3588 | contig516055 | Hsa2 | intergenic LOC344172-KIF5C (LOC391451) | 861 |
| 346019 | SCAFFOLD45848_1894 | contig129146 | no chr. | | 862 |
| 345544 | SCAFFOLD171748_1363 | contig32037 | Hsa12 | intergenic TBX3-LOC440112 | 863 |

Some of these additional positive associations are in the same genomic region or gene as other SNP identified in Table 19, implying multiple hits to the same region and help to confirm that these are true associations. Examples are in the KIF5C region and IMPG2.

TABLE 23
Parallele identifiers for further SNP associated with RBY (retail beef yield)
showing the sequence scaffold, the Baylor College of Medicine sequence contig containing
the SNP, the Genbank Accession of the DNA sequence, and the alternative bases for each
SNP. The exact location of each SNP is shown in the sequence scaffold, the number after
the underscore is the exact base pair from the start of the scaffold.

| ident | Ibiss4 | Sequence scaffold | Baylor Contig | Genbank Accession | SNP bases | SEQ ID NO: |
|---|---|---|---|---|---|---|
| 343676 | Null | SCAFFOLD61161_13201 | Contig264651 | AAFC01264642 | A/G | 864 |
| 343686 | Null | SCAFFOLD70038_5082 | Contig46463 | AAFC01046462 | A/G | 865 |
| 345183 | Null | SCAFFOLD70038_6516 | Contig267483 | AAFC01267474 | A/G | 866 |
| 348558 | Null | SCAFFOLD275605_3060 | Contig307051 | AAFC01307041 | A/G | 867 |
| 348641 | Null | SCAFFOLD310358_3310 | Contig327981 | AAFC01327971 | C/T | 868 |
| 348659 | Null | SCAFFOLD316954_5829 | Contig232078 | AAFC01232072 | A/G | 869 |
| 348797 | Null | SCAFFOLD70054_17340 | Contig359389 | AAFC01359379 | A/G | 870 |
| 349243 | Null | SCAFFOLD215128_19099 | Contig43014 | AAFC01043013 | A/T | 871 |
| 349414 | Null | SCAFFOLD270085_5213 | Contig115177 | AAFC01115173 | A/G | 872 |
| 350070 | Null | SCAFFOLD205652_8347 | Contig119996 | AAFC01119992 | A/G | 873 |
| 350492 | Null | SCAFFOLD70038_4486 | Contig46463 | AAFC01046462 | A/G | 874 |
| 351288 | Null | SCAFFOLD90097_20876 | Contig169910 | AAFC01169904 | A/G | 875 |
| 351296 | Null | SCAFFOLD94123_1093 | Contig235275 | AAFC01235268 | A/C | 876 |
| 351514 | Null | SCAFFOLD192666_11865 | Contig351258 | AAFC01351248 | C/T | 877 |
| 351744 | Null | SCAFFOLD30172_28453 | Contig54331 | AAFC01054330 | A/G | 878 |
| 351886 | Null | SCAFFOLD70038_4640 | Contig46463 | AAFC01046462 | C/T | 879 |
| 354432 | Null | SCAFFOLD201769_1248 | Contig183125 | AAFC01183119 | C/T | 880 |
| 346220 | Null | SCAFFOLD100297_8761 | Contig274152 | AAFC01274143 | G/T | 881 |
| 342814 | Null | SCAFFOLD10496_11993 | Contig353686 | AAFC01353676 | A/G | 882 |
| 345544 | Null | SCAFFOLD171748_1363 | Contig32037 | AAFC01032036 | A/G | 863 |
| 342505 | Null | SCAFFOLD131821_4243 | Contig258386 | AAFC01258377 | G/T | 883 |
| 342541 | Null | SCAFFOLD205782_6486 | Contig337899 | AAFC01337889 | A/G | 884 |
| 343029 | Null | SCAFFOLD29531_1161 | Contig582416 | AAFC01582392 | A/G | 885 |
| 343069 | Null | SCAFFOLD40558_3166 | Contig18928 | AAFC01018928 | A/G | 886 |
| 343107 | Null | SCAFFOLD90080_5962 | Contig222033 | AAFC01222027 | A/G | 887 |
| 343565 | Null | SCAFFOLD30115_1301 | Contig3592 | AAFC01003592 | C/T | 888 |
| 343925 | Null | SCAFFOLD1473_926 | Contig80262 | AAFC01080261 | C/T | 889 |
| 343932 | Null | SCAFFOLD150695_5842 | Contig136621 | AAFC01136616 | A/G | 890 |
| 344010 | Null | SCAFFOLD184185_1569 | Contig181991 | AAFC01181985 | A/G | 891 |
| 344018 | Null | SCAFFOLD186437_1116 | Contig744865 | AAFC01744821 | A/G | 892 |
| 344131 | Null | SCAFFOLD245307_19392 | Contig21810 | AAFC01021810 | C/T | 893 |
| 344183 | Null | SCAFFOLD268752_1509 | Contig85928 | AAFC01085927 | A/T | 894 |
| 344191 | Null | SCAFFOLD270591_7376 | Contig2617 | AAFC01002617 | G/T | 895 |
| 344277 | Null | SCAFFOLD312082_12221 | Contig226381 | AAFC01226375 | A/G | 896 |
| 344411 | Null | SCAFFOLD80071_25898 | Contig29821 | AAFC01029821 | C/G | 897 |
| 344436 | Null | SCAFFOLD92705_1807 | Contig202392 | AAFC01202386 | A/G | 898 |
| 344536 | Null | SCAFFOLD100231_10975 | Contig188868 | AAFC01188862 | C/T | 899 |
| 344701 | Null | SCAFFOLD166104_1102 | Contig160418 | AAFC01160412 | G/T | 900 |
| 344746 | Null | SCAFFOLD195047_10282 | Contig403367 | AAFC01403354 | C/G | 901 |
| 344830 | Null | SCAFFOLD222059_7896 | Contig34733 | AAFC01034732 | C/T | 902 |
| 344838 | Null | SCAFFOLD225628_7865 | Contig115392 | AAFC01115388 | A/G | 903 |
| 344847 | Null | SCAFFOLD230250_13663 | Contig10766 | AAFC01010766 | A/G | 904 |
| 344864 | Null | SCAFFOLD235642_5949 | Contig47850 | AAFC01047849 | C/T | 905 |
| 344973 | Null | SCAFFOLD270296_8097 | Contig517767 | AAFC01517746 | C/T | 906 |
| 344998 | Null | SCAFFOLD282025_3342 | Contig166161 | AAFC01166155 | A/G | 862 |
| 345058 | Null | SCAFFOLD30862_2553 | Contig36867 | AAFC01036866 | C/T | 907 |
| 345165 | Null | SCAFFOLD60719_4705 | Contig149503 | AAFC01149497 | C/T | 908 |
| 345504 | Null | SCAFFOLD160144_12187 | Contig408388 | AAFC01408375 | C/G | 909 |
| 345544 | Null | SCAFFOLD171748_1363 | Contig32037 | AAFC01032036 | A/G | 863 |
| 345555 | Null | SCAFFOLD175439_8685 | Contig435618 | AAFC01435604 | C/T | 910 |
| 345624 | Null | SCAFFOLD205042_52028 | Contig120583 | AAFC01120579 | A/G | 911 |
| 345649 | Null | SCAFFOLD210760_7779 | Contig127376 | AAFC01127371 | C/T | 912 |
| 345669 | Null | SCAFFOLD216080_11820 | Contig200611 | AAFC01200605 | A/G | 913 |
| 345736 | Null | SCAFFOLD241145_9825 | Contig117468 | AAFC01117464 | A/G | 914 |
| 345814 | Null | SCAFFOLD265103_4193 | Contig626589 | AAFC01626561 | A/G | 915 |
| 346006 | Null | SCAFFOLD40979_2070 | Contig251602 | AAFC01251594 | G/T | 916 |
| 346019 | Null | SCAFFOLD45848_1894 | Contig129146 | AAFC01129141 | A/G | 853 |
| 346109 | Null | SCAFFOLD74301_3645 | Contig78575 | AAFC01078574 | C/T | 917 |
| 346122 | Null | SCAFFOLD80060_16638 | Contig78433 | AAFC01078432 | G/T | 918 |
| 346373 | Null | SCAFFOLD15936_11858 | Contig73184 | AAFC01073183 | G/T | 919 |
| 346382 | Null | SCAFFOLD160823_6141 | Contig208493 | AAFC01208487 | A/G | 920 |
| 346426 | Null | SCAFFOLD170731_20633 | Contig543966 | AAFC01543945 | A/G | 921 |
| 346624 | Null | SCAFFOLD246051_2750 | Contig55665 | AAFC01055664 | A/G | 922 |
| 346717 | Null | SCAFFOLD280196_14461 | Contig532607 | AAFC01532586 | C/T | 923 |
| 346742 | Null | SCAFFOLD287116_1821 | Contig62379 | AAFC01062378 | A/T | 924 |
| 346831 | Null | SCAFFOLD317746_15268 | Contig13305 | AAFC01013305 | A/G | 925 |
| 346864 | Null | SCAFFOLD35310_9826 | Contig611044 | AAFC01611017 | C/T | 926 |
| 346881 | Null | SCAFFOLD40305_18584 | Contig217298 | AAFC01217292 | C/T | 927 |
| 346884 | Null | SCAFFOLD40614_7721 | Contig169194 | AAFC01169188 | C/T | 928 |
| 346942 | Null | SCAFFOLD60231_14862 | Contig15101 | AAFC01015101 | C/T | 929 |
| 347013 | Null | SCAFFOLD85585_6225 | Contig128771 | AAFC01128766 | A/G | 930 |
| 347279 | Null | SCAFFOLD168801_862 | Contig407980 | AAFC01407967 | A/C | 931 |
| 347554 | Null | SCAFFOLD260054_24186 | Contig265606 | AAFC01265597 | A/G | 932 |
| 347844 | Null | SCAFFOLD60012_53629 | Contig23420 | AAFC01023420 | C/T | 933 |
| 347846 | Null | SCAFFOLD60231_19610 | Contig736315 | AAFC01736274 | A/G | 934 |
| 348079 | Null | SCAFFOLD11216_1018 | Contig648073 | AAFC01648045 | A/G | 935 |
| 348096 | Null | SCAFFOLD11756_13335 | Contig79774 | AAFC01079773 | A/G | 936 |
| 348115 | Null | SCAFFOLD125326_1284 | Contig569756 | AAFC01569734 | G/T | 937 |
| 348122 | Null | SCAFFOLD126910_2320 | Contig164511 | AAFC01164505 | G/T | 938 |
| 348168 | Null | SCAFFOLD140359_23711 | Contig125039 | AAFC01125034 | C/T | 939 |
| 348287 | Null | SCAFFOLD186806_2279 | Contig239738 | AAFC01239731 | A/G | 940 |
| 348339 | Null | SCAFFOLD205140_17944 | Contig200605 | AAFC01200599 | G/T | 941 |
| 348387 | Null | SCAFFOLD22008_2822 | Contig665733 | AAFC01665704 | A/G | 942 |
| 348452 | Null | SCAFFOLD241780_462 | Contig224973 | AAFC01224967 | C/G | 943 |
| 348549 | Null | SCAFFOLD275031_17117 | Contig425165 | AAFC01425152 | A/G | 944 |
| 348555 | Null | SCAFFOLD275478_9050 | Contig141866 | AAFC01141861 | A/G | 945 |
| 348605 | Null | SCAFFOLD295856_23756 | Contig3923 | AAFC01003923 | A/G | 946 |
| 348607 | Null | SCAFFOLD296686_7831 | Contig64263 | AAFC01064262 | A/G | 947 |
| 348679 | Null | SCAFFOLD333247_299 | Contig771786 | AAFC01771736 | A/G | 948 |
| 348824 | Null | SCAFFOLD81723_6996 | Contig212935 | AAFC01212929 | C/T | 949 |
| 348825 | Null | SCAFFOLD81723_7214 | Contig212935 | AAFC01212929 | C/G | 950 |
| 348976 | Null | SCAFFOLD125326_1171 | Contig569756 | AAFC01569734 | C/T | 951 |
| 349018 | Null | SCAFFOLD141002_4090 | Contig89937 | AAFC01089935 | A/C | 952 |
| 349024 | Null | SCAFFOLD145475_11348 | Contig184223 | AAFC01184217 | A/G | 953 |
| 349040 | Null | SCAFFOLD150293_14373 | Contig130465 | AAFC01130460 | G/T | 954 |
| 349163 | Null | SCAFFOLD190011_29357 | Contig155849 | AAFC01155843 | A/G | 955 |
| 349178 | Null | SCAFFOLD191230_2970 | Contig159461 | AAFC01159455 | G/T | 956 |
| 349457 | Null | SCAFFOLD285252_10781 | Contig438953 | AAFC01438939 | G/T | 957 |
| 349636 | Null | SCAFFOLD55122_7810 | Contig84561 | AAFC01084560 | A/G | 958 |
| 349641 | Null | SCAFFOLD55842_17207 | Contig13161 | AAFC01013161 | C/G | 959 |
| 349728 | Null | SCAFFOLD91511_3588 | Contig276853 | AAFC01276844 | C/T | 858 |
| 349785 | Null | SCAFFOLD100231_10874 | Contig188868 | AAFC01188862 | C/T | 960 |
| 349907 | Null | SCAFFOLD145475_11475 | Contig184223 | AAFC01184217 | C/T | 961 |
| 349919 | Null | SCAFFOLD150945_2291 | Contig473577 | AAFC01473561 | A/T | 962 |
| 350280 | Null | SCAFFOLD285539_13817 | Contig210695 | AAFC01210689 | G/T | 963 |
| 350295 | Null | SCAFFOLD290912_4311 | Contig54538 | AAFC01054537 | A/G | 964 |
| 350397 | Null | SCAFFOLD35092_20269 | Contig288805 | AAFC01288796 | A/T | 965 |
| 350429 | Null | SCAFFOLD49643_12002 | Contig119147 | AAFC01119143 | A/G | 966 |
| 350461 | Null | SCAFFOLD60231_20088 | Contig736315 | AAFC01736274 | A/G | 967 |
| 350495 | Null | SCAFFOLD70270_5697 | Contig249828 | AAFC01249820 | G/T | 968 |
| 350570 | Null | SCAFFOLD94371_446 | Contig754256 | AAFC01754211 | C/G | 969 |
| 350667 | Null | SCAFFOLD121139_3191 | Contig52631 | AAFC01052630 | A/G | 861 |
| 350668 | Null | SCAFFOLD121139_5171 | Contig52631 | AAFC01052630 | C/T | 970 |
| 350780 | Null | SCAFFOLD166988_3684 | Contig10149 | AAFC01010149 | A/G | 971 |
| 351094 | Null | SCAFFOLD305427_17925 | Contig184849 | AAFC01184843 | C/T | 972 |
| 351095 | Null | SCAFFOLD307913_759 | Contig792325 | AAFC01792270 | C/T | 973 |
| 351203 | Null | SCAFFOLD5373_38904 | Contig21156 | AAFC01021156 | A/G | 974 |
| 351219 | Null | SCAFFOLD60231_20057 | Contig736315 | AAFC01736274 | C/T | 975 |
| 351369 | Null | SCAFFOLD110120_25106 | Contig42123 | AAFC01042122 | A/T | 976 |
| 351525 | Null | SCAFFOLD200020_32472 | Contig333666 | AAFC01333656 | C/T | 977 |
| 351572 | Null | SCAFFOLD221335_1225 | Contig60965 | AAFC01060964 | C/T | 978 |
| 351684 | Null | SCAFFOLD275885_2418 | Contig345502 | AAFC01345492 | A/T | 979 |
| 351830 | Null | SCAFFOLD40305_18956 | Contig217298 | AAFC01217292 | A/G | 980 |
| 351850 | Null | SCAFFOLD5176_12230 | Contig48365 | AAFC01048364 | A/G | 981 |
| 351901 | Null | SCAFFOLD80021_27195 | Contig195083 | AAFC01195077 | C/G | 982 |
| 351975 | Null | SCAFFOLD107823_1588 | Contig355581 | AAFC01355571 | A/G | 983 |
| 352026 | Null | SCAFFOLD125326_1056 | Contig569756 | AAFC01569734 | C/T | 984 |
| 352028 | Null | SCAFFOLD125519_9311 | Contig103628 | AAFC01103625 | A/C | 985 |
| 352077 | Null | SCAFFOLD155617_7577 | Contig4087 | AAFC01004087 | A/G | 986 |
| 352133 | Null | SCAFFOLD181569_5642 | Contig17298 | AAFC01017298 | C/T | 987 |
| 352224 | Null | SCAFFOLD240110_25059 | Contig482987 | AAFC01482971 | C/T | 859 |
| 352311 | Null | SCAFFOLD288102_395 | Contig407852 | AAFC01407839 | A/G | 988 |
| 352396 | Null | SCAFFOLD40305_19056 | Contig217298 | AAFC01217292 | C/T | 989 |
| 352445 | Null | SCAFFOLD69981_14378 | Contig82757 | AAFC01082756 | C/T | 990 |
| 352465 | Null | SCAFFOLD80596_16424 | Contig65286 | AAFC01065285 | C/T | 853 |
| 352554 | Null | SCAFFOLD123632_3438 | Contig212890 | AAFC01212884 | C/T | 991 |
| 352556 | Null | SCAFFOLD125013_22381 | Contig151545 | AAFC01151539 | A/C | 992 |
| 352566 | Null | SCAFFOLD131356_3439 | Contig235444 | AAFC01235437 | A/C | 993 |
| 352618 | Null | SCAFFOLD156754_729 | Contig384004 | AAFC01383992 | A/C | 994 |
| 352779 | Null | SCAFFOLD255402_24210 | Contig57357 | AAFC01057356 | C/T | 995 |
| 352877 | Null | SCAFFOLD30708_2335 | Contig186691 | AAFC01186685 | C/T | 854 |
| 352891 | Null | SCAFFOLD318671_297 | Contig422189 | AAFC01422176 | A/G | 996 |
| 352969 | Null | SCAFFOLD80021_27175 | Contig195083 | AAFC01195077 | A/G | 997 |
| 353002 | Null | SCAFFOLD99544_624 | Contig464928 | AAFC01464913 | A/C | 862 |
| 353130 | Null | SCAFFOLD170433_19316 | Contig246027 | AAFC01246019 | A/G | 998 |
| 353161 | Null | SCAFFOLD195717_2054 | Contig217113 | AAFC01217107 | G/T | 999 |
| 353286 | Null | SCAFFOLD291299_3699 | Contig360397 | AAFC01360387 | A/G | 1000 |
| 353471 | Null | SCAFFOLD140124_7042 | Contig170618 | AAFC01170612 | A/G | 1001 |
| 353531 | Null | SCAFFOLD195717_2152 | Contig217113 | AAFC01217107 | A/T | 1002 |
| 353532 | Null | SCAFFOLD195717_2425 | Contig217113 | AAFC01217107 | C/T | 1003 |
| 353682 | Null | SCAFFOLD61490_4909 | Contig6803 | AAFC01006803 | G/T | 1004 |
| 353686 | Null | SCAFFOLD65661_14423 | Contig446928 | AAFC01446914 | A/T | 1005 |
| 353733 | Null | SCAFFOLD100651_19519 | Contig460512 | AAFC01460497 | C/T | 1006 |
| 353802 | Null | SCAFFOLD185286_3978 | Contig509750 | AAFC01509732 | C/T | 1007 |
| 353811 | Null | SCAFFOLD195717_2202 | Contig217113 | AAFC01217107 | A/C | 855 |
| 353853 | Null | SCAFFOLD266133_5948 | Contig153859 | AAFC01153853 | A/G | 1008 |
| 353866 | Null | SCAFFOLD280196_14851 | Contig532607 | AAFC01532586 | A/G | 1009 |
| 354047 | Null | SCAFFOLD280893_11141 | Contig2502 | AAFC01002502 | A/C | 1010 |
| 354061 | Null | SCAFFOLD310542_845 | Contig316380 | AAFC01316370 | C/T | 1011 |
| 354094 | Null | SCAFFOLD80920_10441 | Contig246029 | AAFC01246021 | C/G | 1012 |
| 354108 | Null | SCAFFOLD100305_20665 | Contig12962 | AAFC01012962 | C/T | 1013 |
| 354141 | Null | SCAFFOLD166988_3548 | Contig10149 | AAFC01010149 | A/T | 1014 |
| 354167 | Null | SCAFFOLD236715_293 | Contig355463 | AAFC01355453 | A/C | 1015 |
| 354312 | Null | SCAFFOLD291031_8783 | Contig8197 | AAFC01008197 | A/T | 1016 |
| 354319 | Null | SCAFFOLD314261_290 | Contig68227 | AAFC01068226 | C/T | 1017 |
| 354336 | Null | SCAFFOLD78633_845 | Contig568155 | AAFC01568133 | A/T | 1018 |
| 354465 | Null | SCAFFOLD89532_1964 | Contig202137 | AAFC01202131 | C/T | 1019 |
| 354493 | Null | SCAFFOLD120581_8086 | Contig578507 | AAFC01578483 | A/G | 1020 |

TABLE 24

SNP associated with RBY (retail beef yield)
obtained from the IBISS4 database.

| | |
|---|---|
| IBISS4snpl44 IBISS4 btcn16436 9/16 1400 | A/G |
| GTTTTTGGCTTCAGAGCTGCATACTTGTAGAATTTCACTTGGGATTTCCC | Homo sapiens pyridoxal |
| TAACGGTTACAAACCTGGACCCGCAGGAGGACTGAGGCTCTGACTCAGGC | (pyridoxine, vitamin B6) |
| | kinase (PDXK), |
| | mRNA 0.031 |
| | (SEQ ID NO:1021) |
| IBISS4snp416 IBISS4 btcn24397 11/18 493 | C/T |
| CACTGGAATTCAAAGTATAACAGTTATGCAACTGCTCTGAAGCATGCCGA | Homo sapiens carbonic |
| GGTATAGCTGTGGTTGGGGTTTTTCTGAAG | anhydrase III, muscle specific |
| | (CA3), mRNA0.0 |
| | (SEQ ID NO:1022) |
| IBISS4snp828 IBISS4 btcn43670 6/11 1476 | C/T |
| ATTCTGGATATCATCTTCCGGGACTTCTGCGTGGGCAAGTGAGGGGACAG | Homo sapiens GTP binding |
| GGAAGCTCAGACCCAGGCTGGAGGAGCACCCGGAAGCCTTGGGGTATCTG | protein 3 (mitochondrial) |
| | (GTPBP3), mRNA 0.0 |
| | (SEQ ID NO:1023) |
| IBISS4snp1001 IBISS4 btcn6645 7/11 1142 | T/C |
| TTTCTATTTCTGTAGAGTTTACATGATTTCCCACTGCTTACACTTTAGAA | Homo sapiens |
| GTTTATTTTATGGGGGCTGAGGGATTAAAAGAGTGTGAATGAACAGGTAA | methylenetetrahydrofolate |
| | dehydrogenase (NADP+ dependent) |
| | 1-like (MTHFD1L), mRNA 0.0 |
| | (SEQ ID NO:1024) |
| IBISS4snp1073 IBISS4 btcn8191 5/10 1410 | T/C |
| GTATGTTTGTGTAACACGCAGATGTGTGTTATATTTGAAAGTGATGACTC | Homo sapiens hypothetical protein |
| TGTGAAATGCTAGGAATGTATCGTGTAATTTCTAAAACCTGTGATGCCGT | MGC8721 (MGC8721), |
| | mRNA 1e−108 |
| | (SEQ ID NO:1025) |

### EXAMPLE 7

To find additional loci affecting retail beef yield, further analyses of the whole genome scan were performed. First, the RBY trait values for all measured individuals, not just the 189 in the whole genome scan, were adjusted using the model rby ˜ mu herd kill_group age ! sireid using ASREML. The residual RBY phenotypes were then available for further analysis. The means and standard errors of the residual RBY for each genotype in the whole genome scan were calculated and at test computed for each comparison. The statistical significance of the largest mean difference was calculated using 100,000 permutations.

TABLE 25
A set of loci with high levels of support associated with residual retail beef
yield. Snp_id is the ParAllele SNP identifier, scaffold-v2 is the version 2 scaffold,
bp is the location of the SNP in the scaffold, scaffold-v1/IBISS4 gives a direct
reference to the SNP, Hsa is the human chromosome associated with the SNP and Gene is
the closest gene to the SNP.

| | | | | | | SEQ |
|---|---|---|---|---|---|---|
| | scaffold- | | | | | ID |
| snp_id | v2 | bp | IBISS4/scaffold-v1 | Hsa | Gene | NO. |
| 343447 | 5.178 | 319773 | SCAFFOLD222082_22390 | 22 | TNRC6B trinucleotide repeat containing 6B | 1026 |
| 353694 | Un.112 | 731683 | SCAFFOLD71700_1626 | 9 | DEC1 deleted in esophageal cancer 1 | 1027 |
| 348480 | Un.2641 | 29191 | SCAFFOLD25364_23392 | 18 | SERPINB10 serine/cysteine proteinase inhibitor, clade B | 1028 |
| | | | | | (ovalbumin), member 10 | |
| 353782 | 27.16 | 748646 | SCAFFOLD155902_5584 | 4 | LOC442118 similar to ODZ3 odd Oz/ten-m homolog 3 | 1029 |
| 350348 | Un.8313 | 33029 | SCAFFOLD311898_4113 | 9 | not near gene | 1030 |
| 348870 | 10.13 | 4702 | IBISS4snp305 | 15 | MGC4562 hypothetical protein | 1031 |
| 345294 | 8.92 | 55081 | IBISS4snp199 | 8 | MGC29816 hypothetical protein | 1032 |
| 346290 | Un.426 | 474982 | SCAFFOLD126235_9752 | 1 | not near gene | 1033 |
| 348610 | Un.407 | 201779 | SCAFFOLD300039_10030 | 1 | FLJ10157 hypothetic protein near SMYD3 SET and MYND | 1034 |
| | | | | | domain containing 3 | |
| 349330 | 10.146 | NULL | SCAFFOLD240809_6669 | 14 | not near gene | 1035 |
| 352978 | 10.105 | 287828 | SCAFFOLD81450_1736 | 15 | between CEP152 (KIAA0912) and KIAA0256 hypothetical | 1036 |
| | | | | | genes | |
| 353814 | Un.12 | 1348480 | SCAFFOLD201310_4882 | 3 | DSCR5 Down syndrome critical region gene 5 | 1037 |

The results of this analysis indicates a further 145 loci of interest (P < 0.01) (Tables 26 and 27) and the top loci not previously covered are shown annotated in Table 25.

TABLE 26
Loci not previously described with association to RBY. Associations between DNA
markers and retail beef yield sorted in decreasing order of statistical significance.
Locus is the ParAllele identifiers of the polymorphisms, N is the number of genotypes,
mean_0 is the mean retail beef yield for genotype 0, mean_1 is the mean for genotype 1, and
mean_2 is the mean of genotype 2, SE is the standard error, a is the additive effect, k is
the dominance effect, alpha is the average effect of allele substitutions, tmax is the
value of the t test, and PermP is the P value determined from 100,000 permutation tests
expressed as a positive integer. A minus value for a means the mean of genotype 0 is lower
than the mean of genotype 2, and a minus value for alpha means that selecting for allele 0
will reduce the average values of retail beef yield in the population. Scaffold-v2 is the
Draft version 2 Bovine genome sequence, bp is the location in base pairs in the scaffold,
and bases are the alternative bases.

| snp_id | scaffold-v2 | bp | bases | mean0 | SE | mean1 | SE | mean2 | SE |
|---|---|---|---|---|---|---|---|---|---|
| 343447 | 5.178 | 319773 | A/G | 0.56 | 0.33 | 0.53 | 0.49 | −1.34 | 0.31 |
| 348480 | Un.2641 | 29191 | A/G | −0.17 | 0.32 | −0.08 | 0.34 | 3.96 | 1.44 |
| 353694 | Un.112 | 731683 | A/C | 4.23 | 1.36 | −0.01 | 0.33 | −0.26 | 0.32 |
| 350348 | Un.8313 | 33029 | C/T | −0.86 | 0.35 | 1.56 | 0.53 | 0.10 | 0.47 |
| 353782 | 27.16 | 748646 | A/T | −0.03 | 0.24 | 4.18 | 1.88 | 0.13 | 1.03 |
| 348870 | 10.13 | 4702 | C/T | 3.17 | 1.57 | −0.81 | 0.34 | 0.54 | 0.34 |
| 349330 | NULL | NULL | G/T | −1.26 | 0.34 | 0.96 | 0.41 | −0.10 | 0.42 |
| 352978 | 10.105 | 287828 | G/T | −0.63 | 0.26 | 0.85 | 0.62 | 1.49 | 0.46 |
| 345294 | 8.92 | 55081 | C/T | 4.28 | 1.93 | −0.65 | 0.42 | 0.22 | 0.30 |
| 346290 | Un.426 | 474982 | A/G | −0.03 | 0.25 | 3.73 | 1.87 | NaN | NaN |
| 348610 | Un.407 | 201779 | C/T | 0.28 | 0.36 | 0.63 | 0.32 | −1.67 | 0.53 |
| 353814 | Un.12 | 1348480 | C/T | 0.05 | 0.35 | −0.60 | 0.33 | 2.04 | 0.81 |

| snp_id | N | Freq | a | k | α | tmax | PermP | |
|---|---|---|---|---|---|---|---|---|
| 343447 | 70 | 0.58 | 0.95 | 0.97 | 1.09 | 3.78 | 0.0001 | SEQ ID NO: 1026 |
| 348480 | 70 | 0.70 | −2.06 | 0.96 | −2.85 | 4.22 | 0.0001 | SEQ ID NO: 1028 |
| 353694 | 70 | 0.29 | 2.25 | −0.89 | 3.10 | 4.59 | 0.0001 | SEQ ID NO: 1027 |
| 350348 | 62 | 0.42 | −0.48 | −4.04 | −0.79 | 3.88 | 0.0003 | SEQ ID NO: 1030 |
| 353782 | 69 | 0.91 | −0.08 | −53.30 | 3.33 | 4.10 | 0.0003 | SEQ ID NO: 1029 |
| 348870 | 70 | 0.25 | 1.32 | −2.03 | 2.65 | 3.85 | 0.0004 | SEQ ID NO: 1031 |
| 349330 | 70 | 0.48 | −0.58 | −2.84 | −0.65 | 3.42 | 0.0004 | SEQ ID NO: 1035 |
| 352978 | 68 | 0.73 | −1.06 | −0.39 | −0.87 | 3.52 | 0.0004 | SEQ ID NO: 1036 |
| 345294 | 68 | 0.18 | 2.03 | −1.43 | 3.90 | 3.92 | 0.0006 | SEQ ID NO: 1032 |
| 346290 | 67 | 0.97 | NaN | NaN | NaN | 3.46 | 0.0008 | SEQ ID NO: 1033 |
| 348610 | 70 | 0.79 | 0.98 | 1.36 | 1.74 | 3.83 | 0.0008 | SEQ ID NO: 1034 |
| 353814 | 70 | 0.66 | −0.99 | 1.66 | −1.51 | 3.60 | 0.0010 | SEQ ID NO: 1037 |

TABLE 27
Further loci associated with RBY. Associations between DNA markers and
retail beef yield sorted in decreasing order of statistical significance. Locus is
the ParAllele identifiers of the polymorphisms, N is the number of genotypes, mean_0
is the mean retail beef yield for genotype 0, mean_1 is the mean for genotype 1, and
mean_2 is the mean of genotype 2, SE is the standard error, a is the additive effect,
k is the dominance effect, alpha is the average effect of allele substitutions, tmax
is the value of the t test, and PermP is the P value determined from 100,000
permutation tests expressed as a positive integer. A minus value for a means the mean
of genotype 0 is lower than the mean of genotype 2, and a minus value for alpha means
that selecting for allele 0 will reduce the average values of retail beef yield in
the population. Scaffold-v2 is the Draft version 2 Bovine genome sequence, bp is the
location in base pairs in the scaffold, and bases are the alternative bases.

| snp_id | scaffold-v2 | bp | bases | mean0 | SE | mean1 | SE | mean2 | SE |
|---|---|---|---|---|---|---|---|---|---|
| 354343 | Un.4413 | 30649 | C/T | −0.09 | 0.53 | 1.01 | 0.42 | −0.84 | 0.34 |
| 344060 | 20.7 | 760485 | C/T | −0.26 | 0.30 | −0.04 | 0.40 | 2.68 | 1.00 |
| 352157 | Un.12 | 1348535 | A/G | 0.05 | 0.35 | −0.60 | 0.33 | 2.04 | 0.81 |
| 353969 | Un.409 | 23076 | C/T | 2.98 | 1.27 | 0.17 | 0.50 | −0.24 | 0.28 |
| 344263 | 1.271 | 88166 | A/G | −0.04 | 0.26 | 2.51 | 0.87 | −2.15 | 0.67 |
| 350078 | 20.7 | 760555 | G/T | −0.33 | 0.30 | 0.04 | 0.40 | 2.68 | 1.00 |
| 353358 | 4.14 | 285151 | C/G | 1.92 | 0.58 | −0.76 | 0.48 | 0.05 | 0.38 |
| 351379 | Un.4413 | 30398 | A/G | −0.09 | 0.53 | 1.01 | 0.42 | −0.84 | 0.34 |
| 345474 | Un.129 | 166181 | C/T | 0.33 | 0.32 | −0.77 | 0.47 | 2.06 | 0.48 |
| 348027 | 4.25 | 162574 | A/C | −2.13 | 0.51 | 0.63 | 0.40 | 0.25 | 0.34 |
| 342893 | Un.1068 | 266682 | G/T | 0.52 | 0.44 | 0.09 | 0.28 | −3.07 | 0.40 |
| 343143 | 2.193 | 674112 | A/G | 3.30 | 1.82 | −0.27 | 0.34 | 0.20 | 0.31 |
| 343973 | Un.1068 | 266615 | A/C | 0.52 | 0.44 | 0.09 | 0.28 | −3.07 | 0.40 |
| 349820 | 25.17 | 595288 | C/T | −0.08 | 0.24 | 2.38 | 1.37 | NaN | NaN |
| 349318 | 29.17 | 156519 | C/T | 0.03 | 0.27 | 3.94 | 2.15 | −1.07 | 1.57 |
| 347219 | Un.653 | 125734 | C/G | 2.72 | 1.23 | −1.57 | 0.52 | 0.22 | 0.29 |
| 344686 | 7.42 | 254134 | A/G | 0.37 | 0.34 | 0.76 | 0.52 | −1.52 | 0.38 |
| 348042 | 16.129 | 249227 | C/T | −0.59 | 0.33 | 0.95 | 0.38 | −0.21 | 0.77 |
| 351653 | 17.48 | 444119 | A/G | 0.37 | 0.46 | −0.55 | 0.33 | 1.77 | 0.61 |
| 349147 | Un.5811 | 49140 | A/T | 0.23 | 0.28 | −2.02 | 0.93 | 1.10 | 0.82 |
| 348078 | 15.108 | 11460 | C/T | 0.56 | 0.37 | −0.87 | 0.35 | 1.52 | 0.80 |
| 352491 | 12.114 | 127963 | C/T | 2.53 | 0.83 | −0.15 | 0.40 | −0.13 | 0.34 |
| 353167 | 19.36 | 9172 | A/G | 2.60 | 1.70 | −0.46 | 0.27 | 0.51 | 0.44 |
| 351531 | Un.545 | 89148 | A/G | NaN | NaN | 2.98 | 0.99 | −0.01 | 0.27 |
| 347102 | 16.129 | 249248 | A/G | −0.21 | 0.77 | 0.96 | 0.39 | −0.59 | 0.33 |
| 349796 | 26.1 | 24979 | G/T | 0.66 | 0.55 | −0.78 | 0.30 | 0.86 | 0.50 |
| 344637 | 9.105 | 303802 | A/G | 0.57 | 0.32 | −1.12 | 0.32 | NaN | NaN |
| 352453 | 9.49 | 251562 | A/G | −0.65 | 0.40 | −0.02 | 0.39 | 1.26 | 0.52 |
| 352213 | Un.5050 | 37699 | C/G | 1.13 | 0.49 | −0.76 | 0.37 | 0.44 | 0.45 |
| 352924 | 15.119 | 50050 | A/G | 0.06 | 0.30 | 0.01 | 0.60 | 2.87 | 0.38 |
| 347799 | 8.1 | 110363 | A/G | −0.22 | 0.33 | 0.16 | 0.35 | 2.97 | 2.05 |
| 353777 | Un.6304 | 40244 | A/G | −0.01 | 0.25 | 3.28 | 1.76 | −0.48 | NaN |
| 354162 | Un.3082 | 36175 | G/T | −2.14 | 0.26 | 0.86 | 0.39 | 0.01 | 0.37 |
| 349265 | Un.1096 | 266107 | C/T | 1.59 | 0.83 | −0.44 | 0.29 | 0.33 | 0.43 |
| 350665 | 7.13 | 230206 | A/G | −0.87 | 0.73 | −1.02 | 0.50 | 0.85 | 0.33 |
| 348488 | Un.1500 | 17313 | C/G | 0.19 | 0.62 | 0.58 | 0.33 | −1.17 | 0.37 |
| 351488 | 12.116 | 226560 | C/T | 0.08 | 0.39 | 0.14 | 0.34 | 3.37 | 0.60 |
| 345275 | Un.5870 | 16352 | G/T | 0.66 | 0.57 | −1.40 | 0.37 | 0.37 | 0.32 |
| 346657 | Un.433 | 207857 | C/G | 2.33 | 0.56 | −0.59 | 0.41 | 0.29 | 0.35 |
| 351225 | 14.83 | 392261 | G/T | 0.45 | 0.40 | −0.63 | 0.34 | 1.47 | 0.74 |
| 353475 | 8.2 | 73914 | A/C | −0.25 | 0.32 | −0.11 | 0.43 | 1.86 | 0.84 |
| 350926 | 10.146 | 523255 | A/T | −1.24 | 0.38 | 0.53 | 0.37 | 0.56 | 0.53 |
| 352221 | Un.4383 | 55920 | A/T | 0.25 | 0.31 | 0.40 | 0.46 | −1.57 | 0.45 |
| 346891 | 16.31 | 307800 | A/G | 1.26 | 0.56 | −0.44 | 0.33 | −0.03 | 0.54 |
| 350862 | 9.87 | 1287524 | C/T | 0.60 | 0.31 | −1.24 | 0.42 | 0.22 | 1.26 |
| 352012 | Un.968 | 241970 | A/G | 0.33 | 0.53 | −0.55 | 0.32 | 1.21 | 0.55 |
| 353504 | Un.974 | 227042 | A/G | −0.39 | 0.25 | 1.36 | 0.68 | 0.55 | 0.82 |
| 354195 | NULL | NULL | A/T | 0.57 | 0.34 | −0.96 | 0.35 | 1.79 | 1.31 |
| 345778 | Un.4647 | 26812 | C/T | 1.97 | 0.83 | 0.18 | 0.45 | −0.30 | 0.32 |
| 346928 | 15.56 | 738365 | A/G | −3.76 | NaN | 1.46 | 0.49 | −0.28 | 0.29 |
| 348864 | 5.175 | 82168 | A/G | 2.91 | 1.44 | −0.33 | 0.37 | 0.14 | 0.36 |
| 346477 | 18.21 | 504871 | A/C | 0.08 | 0.90 | 1.19 | 0.63 | −0.48 | 0.28 |
| 347589 | 12.105 | 744521 | C/T | 0.20 | 0.31 | −0.57 | 0.37 | 1.14 | 0.55 |
| 348449 | 10.146 | 523467 | C/T | 0.49 | 0.51 | 0.60 | 0.39 | −1.24 | 0.38 |
| 352697 | Un.12 | 1348435 | C/G | −0.07 | 0.32 | −0.51 | 0.39 | 2.04 | 0.89 |
| 349052 | 14.79 | 314697 | G/T | 0.31 | 1.52 | −0.94 | 0.35 | 0.60 | 0.33 |
| 343517 | Un.2305 | 108919 | A/G | −0.73 | 0.31 | 2.14 | 3.03 | 0.60 | 0.34 |
| 347425 | 25.88 | 445813 | A/G | 0.88 | 0.41 | −0.97 | 0.40 | 0.23 | 0.52 |
| 348867 | 3.166 | 23849 | C/T | 2.51 | 1.12 | −0.72 | 0.49 | 0.22 | 0.31 |
| 353212 | 10.146 | 525357 | C/G | 0.17 | 0.49 | 0.61 | 0.37 | −1.30 | 0.45 |
| 346115 | 2.72 | 433544 | C/T | 3.06 | 1.70 | 0.71 | 0.46 | −0.24 | 0.29 |
| 346232 | Un.159 | 456024 | A/G | −0.16 | 0.33 | −0.19 | 0.38 | 1.98 | 0.91 |
| 346976 | 14.79 | 311117 | C/G | 0.60 | 0.33 | −0.94 | 0.35 | 0.31 | 1.52 |
| 346890 | 16.31 | 310948 | A/G | 1.26 | 0.56 | −0.40 | 0.32 | −0.03 | 0.54 |
| 343386 | Un.4575 | 30175 | C/T | −1.75 | 0.43 | 1.16 | 0.60 | 0.16 | 0.31 |
| 343526 | 8.128 | 88682 | C/T | NaN | NaN | 3.76 | 2.17 | 0.01 | 0.25 |
| 348771 | 16.18 | 290928 | A/C | 0.32 | 0.38 | 0.52 | 0.35 | −1.57 | 0.66 |
| 352285 | 22.45 | 420733 | C/G | 2.05 | 0.99 | −0.18 | 0.57 | −0.08 | 0.28 |
| 343589 | NULL | NULL | C/T | 0.84 | 0.32 | −0.69 | 0.47 | −0.78 | 0.91 |
| 346225 | Un.3643 | 40063 | A/G | −0.20 | 1.54 | 1.43 | 0.71 | −0.27 | 0.26 |
| 346963 | 6.197 | 964490 | C/T | −0.76 | 0.70 | 1.45 | 0.61 | −0.20 | 0.29 |
| 347235 | 11.185 | 98871 | C/T | −0.15 | 0.28 | 0.58 | 0.50 | 3.31 | 2.65 |
| 352932 | Un.2934 | 66595 | C/T | 0.66 | 0.36 | −0.86 | 0.35 | 1.09 | 1.21 |
| 344987 | Un.763 | 211063 | C/T | 0.41 | 0.28 | −1.74 | 0.56 | NaN | NaN |
| 345162 | 14.43 | 596224 | A/G | 1.02 | 0.50 | −0.50 | 0.30 | 0.73 | 0.82 |
| 353824 | Un.217 | 382674 | A/G | 0.70 | 0.55 | −0.57 | 0.31 | 0.96 | 0.48 |
| 342514 | 28.2 | 122416 | C/T | 1.53 | 1.08 | −0.73 | 0.34 | 0.29 | 0.35 |
| 350192 | Un.433 | 207646 | C/T | 2.33 | 0.56 | −0.59 | 0.41 | 0.31 | 0.34 |
| 352518 | Un.3536 | 57752 | C/T | −0.06 | 0.26 | 2.22 | 1.12 | −0.14 | 3.02 |
| 354161 | Un.3082 | 36240 | C/T | 0.04 | 0.38 | 0.83 | 0.39 | −1.38 | 0.58 |
| 344034 | Un.1794 | 114318 | C/T | −1.20 | 1.23 | 1.45 | 0.61 | −0.46 | 0.31 |
| 343548 | Un.2274 | 24581 | A/G | −0.46 | NaN | 3.24 | 1.69 | 0.03 | 0.25 |
| 346163 | 22.96 | 5306 | A/G | 1.07 | 0.47 | −1.61 | 0.62 | 0.35 | 0.31 |
| 347731 | 23.89 | 22357 | C/T | −0.10 | 0.41 | 2.57 | 1.15 | −0.04 | 0.27 |
| 347988 | 4.136 | 966787 | G/T | 1.11 | 0.58 | −0.57 | 0.30 | 0.12 | 0.44 |
| 346563 | 20.29 | 1125490 | C/T | −0.02 | 0.29 | 0.21 | 0.36 | 3.52 | 2.31 |
| 349526 | Un.321 | 388395 | A/C | 1.33 | 0.69 | −0.59 | 0.32 | 0.26 | 0.37 |
| 350110 | Un.1295 | 148066 | C/T | 0.23 | 0.32 | −0.63 | 0.49 | 2.36 | 0.83 |
| 346359 | 11.185 | 98726 | A/G | 3.31 | 2.65 | 0.58 | 0.50 | −0.15 | 0.28 |
| 344197 | 10.112 | 549674 | A/G | −0.17 | 0.26 | 1.69 | 0.74 | −2.78 | NaN |
| 345543 | 11.14 | 100358 | G/T | 0.11 | 0.56 | −0.69 | 0.33 | 0.84 | 0.43 |
| 348364 | 22.52 | 8443 | A/G | 2.24 | 1.22 | −0.45 | 0.34 | 0.39 | 0.38 |
| 349329 | 10.146 | 525762 | C/T | 0.25 | 0.46 | 0.61 | 0.37 | −1.30 | 0.45 |
| 352030 | Un.2336 | 110962 | C/T | −0.37 | 0.53 | 1.03 | 0.42 | −0.55 | 0.38 |
| 349054 | 6.132 | 410321 | A/G | −0.14 | 1.25 | 1.20 | 0.49 | −0.32 | 0.31 |
| 346097 | 29.2 | 47615 | G/T | 0.77 | 0.69 | −0.45 | 0.30 | 1.09 | 0.51 |
| 347411 | 11.38 | 390008 | C/T | −0.52 | 0.34 | 1.15 | 0.51 | 0.04 | 0.51 |
| 344199 | 24.77 | 1186297 | C/G | −0.07 | 0.44 | −0.39 | 0.35 | 1.48 | 0.66 |
| 348199 | 6.51 | 218288 | A/C | 0.36 | 0.10 | 1.25 | 0.59 | −0.29 | 0.28 |
| 353569 | 10.146 | 523534 | A/G | −1.30 | 0.45 | 0.60 | 0.38 | 0.25 | 0.46 |
| 344596 | 3.99 | 215890 | A/G | 0.38 | 0.56 | −0.64 | 0.40 | 0.81 | 0.34 |
| 353284 | Un.4 | 943455 | G/T | −0.39 | 0.28 | 1.14 | 0.55 | 1.24 | 0.80 |
| 352909 | 14.43 | 586581 | C/G | 0.89 | 0.53 | −0.59 | 0.28 | 0.90 | 0.69 |
| 343004 | 12.107 | 290962 | C/T | −0.14 | 0.33 | −0.07 | 0.36 | 2.29 | 1.24 |
| 351453 | 29.18 | 185260 | A/G | 0.16 | 0.34 | −0.21 | 0.36 | 2.86 | 1.93 |
| 351946 | Un.68 | 155551 | C/T | −1.18 | 0.62 | 1.19 | 0.53 | −0.01 | 0.32 |
| 348729 | 6.128 | 348301 | A/C | 0.17 | 0.31 | −0.45 | 0.48 | 2.60 | 1.16 |
| 349694 | Un.241 | 15362 | C/T | 2.04 | 1.27 | −0.84 | 0.46 | 0.33 | 0.31 |
| 352290 | 2.34 | 19014 | C/T | 2.50 | 1.37 | 0.07 | 0.33 | −0.25 | 0.34 |
| 353571 | 9.16 | 701082 | C/T | 3.70 | 2.34 | −0.26 | 0.68 | 0.06 | 0.27 |
| 346774 | 18.14 | 693589 | A/G | −0.88 | 0.59 | 0.76 | 0.30 | 0.05 | 0.46 |
| 342952 | 25.44 | 4857 | C/T | 3.15 | 1.76 | −0.61 | 0.68 | 0.16 | 0.28 |
| 351900 | 20.7 | 780383 | A/G | −0.58 | 1.64 | −0.77 | 0.33 | 0.71 | 0.35 |
| 353979 | 13.162 | 224309 | G/T | 1.16 | 2.37 | 1.05 | 0.43 | −0.34 | 0.30 |
| 351500 | Un.60 | 811877 | A/G | 0.29 | 0.33 | −0.61 | 0.39 | 1.27 | 0.52 |
| 343483 | 10.156 | 419505 | C/G | −0.28 | 0.27 | 1.34 | 0.69 | 1.38 | NaN |
| 345229 | Un.5650 | 47471 | C/T | 1.18 | 0.53 | −0.40 | 0.32 | 0.25 | 0.68 |
| 348702 | 5.183 | 177029 | C/T | −0.05 | 0.35 | −0.38 | 0.36 | 1.62 | 0.73 |
| 352790 | 5.175 | 641378 | A/G | 1.45 | 0.80 | −0.81 | 0.33 | 0.17 | 0.29 |
| 346071 | 28.4 | 443112 | A/G | 0.21 | 0.53 | −0.72 | 0.39 | 0.87 | 0.43 |
| 343443 | 2.92 | 500532 | A/G | 0.35 | 0.29 | −1.64 | 0.50 | 1.12 | 1.62 |
| 353654 | 5.183 | 176974 | C/T | −0.05 | 0.35 | −0.34 | 0.38 | 1.80 | 0.76 |
| 348109 | 6.2 | 192460 | G/T | −0.94 | 0.28 | 0.70 | 0.38 | 0.05 | 0.75 |
| 352690 | Un.4452 | 47008 | C/T | −0.35 | 0.93 | −0.59 | 0.35 | 0.81 | 0.39 |
| 347812 | 1.184 | 88793 | C/G | −0.42 | 0.32 | 1.16 | 0.60 | 0.22 | 0.72 |
| 349320 | 18.51 | 986902 | A/G | 0.18 | 0.40 | −0.30 | 0.38 | 1.88 | 0.60 |
| 351125 | NULL | NULL | A/G | 0.24 | 0.33 | −0.65 | 0.43 | 1.97 | 0.89 |
| 345061 | Un.3252 | 46984 | A/T | −0.09 | 0.26 | 1.71 | 0.85 | −1.82 | 0.76 |
| 345972 | 30.75 | 281522 | C/T | NaN | NaN | 2.56 | 1.86 | −0.02 | 0.24 |
| 349045 | 14.88 | 143850 | A/C | 0.03 | 0.27 | −0.07 | 0.72 | 2.57 | 2.06 |
| 348554 | 1.18 | 19430 | A/T | −0.16 | 0.24 | 2.00 | 1.31 | 1.72 | 1.77 |
| 349093 | 16.69 | 106244 | A/G | 1.12 | 0.40 | −0.44 | 0.37 | 0.34 | 0.44 |

| snp_id | N | Freq | a | k | α | tmax | PermP | |
|---|---|---|---|---|---|---|---|---|
| 354343 | 70 | 0.44 | 0.37 | 3.95 | 0.20 | 3.20 | 0.0009 | SEQ ID NO: 1038 |
| 344060 | 70 | 0.67 | −1.47 | 0.85 | −1.90 | 3.79 | 0.0010 | SEQ ID NO: 1039 |
| 352157 | 70 | 0.66 | −0.99 | 1.66 | −1.51 | 3.60 | 0.0010 | SEQ ID NO: 1040 |
| 353969 | 69 | 0.23 | 1.61 | −0.74 | 2.25 | 3.64 | 0.0010 | SEQ ID NO: 1041 |
| 344263 | 70 | 0.88 | 1.06 | 3.42 | 3.79 | 3.51 | 0.0011 | SEQ ID NO: 1042 |
| 350078 | 70 | 0.68 | −1.51 | 0.75 | −1.91 | 3.88 | 0.0013 | SEQ ID NO: 1043 |
| 353358 | 64 | 0.30 | 0.93 | −1.87 | 1.62 | 3.54 | 0.0013 | SEQ ID NO: 1044 |
| 351379 | 70 | 0.44 | 0.37 | 3.95 | 0.20 | 3.20 | 0.0014 | SEQ ID NO: 1045 |
| 345474 | 70 | 0.85 | −0.86 | 2.27 | −2.24 | 2.58 | 0.0015 | SEQ ID NO: 1046 |
| 348027 | 68 | 0.16 | −1.19 | −1.32 | −2.25 | 3.46 | 0.0015 | SEQ ID NO: 1047 |
| 342893 | 70 | 0.73 | 1.80 | 0.76 | 2.42 | 3.35 | 0.0016 | SEQ ID NO: 1048 |
| 343143 | 66 | 0.24 | 1.55 | −1.30 | 2.59 | 3.31 | 0.0016 | SEQ ID NO: 1049 |
| 343973 | 70 | 0.73 | 1.80 | 0.76 | 2.42 | 3.35 | 0.0017 | SEQ ID NO: 1050 |
| 349820 | 70 | 0.95 | NaN | NaN | NaN | 2.91 | 0.0017 | SEQ ID NO: 1051 |
| 349318 | 66 | 0.95 | 0.55 | 8.10 | 4.54 | 3.07 | 0.0019 | SEQ ID NO: 1052 |
| 347219 | 68 | 0.09 | 1.25 | −2.44 | 3.75 | 3.88 | 0.0020 | SEQ ID NO: 1053 |
| 344686 | 69 | 0.64 | 0.94 | 1.42 | 1.33 | 3.14 | 0.0022 | SEQ ID NO: 1054 |
| 348042 | 70 | 0.58 | −0.19 | −7.12 | 0.02 | 2.97 | 0.0022 | SEQ ID NO: 1055 |
| 351653 | 68 | 0.65 | −0.70 | 2.31 | −1.20 | 3.41 | 0.0023 | SEQ ID NO: 1056 |
| 349147 | 70 | 0.94 | −0.44 | 6.13 | −2.78 | 2.52 | 0.0024 | SEQ ID NO: 1057 |
| 348078 | 70 | 0.73 | −0.48 | 3.99 | −1.35 | 3.08 | 0.0025 | SEQ ID NO: 1058 |
| 352491 | 70 | 0.33 | 1.33 | −1.01 | 1.79 | 3.38 | 0.0025 | SEQ ID NO: 1059 |
| 353167 | 69 | 0.33 | 1.05 | −1.93 | 1.72 | 3.26 | 0.0025 | SEQ ID NO: 1060 |
| 351531 | 67 | 0.04 | NaN | NaN | NaN | 3.05 | 0.0028 | SEQ ID NO: 1061 |
| 347102 | 69 | 0.42 | 0.19 | 7.18 | −0.03 | 2.95 | 0.0029 | SEQ ID NO: 1062 |
| 349796 | 70 | 0.49 | −0.10 | 15.92 | −0.07 | 2.93 | 0.0029 | SEQ ID NO: 1063 |
| 344637 | 69 | 0.88 | NaN | NaN | NaN | 2.84 | 0.0031 | SEQ ID NO: 1064 |
| 352453 | 70 | 0.51 | −0.96 | 0.34 | −0.97 | 2.94 | 0.0032 | SEQ ID NO: 1065 |
| 352213 | 70 | 0.41 | 0.35 | −4.48 | 0.63 | 3.12 | 0.0033 | SEQ ID NO: 1066 |
| 352924 | 70 | 0.83 | −1.41 | 1.03 | −2.36 | 2.30 | 0.0033 | SEQ ID NO: 1067 |
| 347799 | 70 | 0.67 | −1.60 | 0.76 | −2.02 | 2.87 | 0.0034 | SEQ ID NO: 1068 |
| 353777 | 70 | 0.96 | 0.23 | 15.14 | 3.46 | 3.02 | 0.0034 | SEQ ID NO: 1069 |
| 354162 | 69 | 0.23 | −1.08 | −1.78 | −2.11 | 3.06 | 0.0035 | SEQ ID NO: 1070 |
| 349265 | 70 | 0.47 | 0.63 | −2.22 | 0.71 | 2.94 | 0.0036 | SEQ ID NO: 1071 |
| 350665 | 66 | 0.21 | −0.86 | 1.18 | −0.28 | 3.01 | 0.0036 | SEQ ID NO: 1072 |
| 348488 | 70 | 0.56 | 0.68 | 1.58 | 0.80 | 2.80 | 0.0039 | SEQ ID NO: 1073 |
| 351488 | 65 | 0.78 | −1.64 | 0.97 | −2.52 | 3.34 | 0.0039 | SEQ ID NO: 1074 |
| 345275 | 68 | 0.16 | 0.15 | −12.97 | 1.44 | 3.02 | 0.0041 | SEQ ID NO: 1075 |
| 346657 | 69 | 0.20 | 1.02 | −1.86 | 2.15 | 3.02 | 0.0041 | SEQ ID NO: 1076 |
| 351225 | 70 | 0.72 | −0.51 | 3.13 | −1.22 | 2.83 | 0.0041 | SEQ ID NO: 1077 |
| 353475 | 68 | 0.63 | −1.05 | 0.87 | −1.30 | 2.92 | 0.0041 | SEQ ID NO: 1078 |
| 350926 | 69 | 0.44 | −0.90 | −0.97 | −1.00 | 2.82 | 0.0042 | SEQ ID NO: 1079 |
| 352221 | 68 | 0.67 | 0.91 | 1.17 | 1.27 | 2.94 | 0.0042 | SEQ ID NO: 1080 |
| 346891 | 69 | 0.51 | 0.64 | −1.64 | 0.61 | 2.83 | 0.0043 | SEQ ID NO: 1081 |
| 350862 | 70 | 0.86 | 0.19 | −8.81 | −0.99 | 3.02 | 0.0045 | SEQ ID NO: 1082 |
| 352012 | 70 | 0.52 | −0.44 | 2.99 | −0.50 | 2.98 | 0.0046 | SEQ ID NO: 1083 |
| 353504 | 69 | 0.79 | −0.47 | −2.72 | 0.27 | 2.98 | 0.0046 | SEQ ID NO: 1084 |
| 354195 | 68 | 0.79 | −0.61 | 3.52 | −1.87 | 2.90 | 0.0046 | SEQ ID NO: 1085 |
| 345778 | 68 | 0.34 | 1.14 | −0.58 | 1.35 | 3.12 | 0.0047 | SEQ ID NO: 1086 |
| 346928 | 70 | 0.16 | −1.74 | −2.00 | −4.12 | 3.18 | 0.0048 | SEQ ID NO: 1087 |
| 348864 | 68 | 0.26 | 1.39 | −1.33 | 2.26 | 3.15 | 0.0048 | SEQ ID NO: 1088 |
| 346477 | 62 | 0.27 | 0.28 | 4.90 | −0.34 | 2.83 | 0.0049 | SEQ ID NO: 1089 |
| 347589 | 70 | 0.45 | −0.47 | 2.64 | −0.34 | 2.67 | 0.0049 | SEQ ID NO: 1090 |
| 348449 | 68 | 0.57 | 0.87 | 1.12 | 0.99 | 2.88 | 0.0049 | SEQ ID NO: 1091 |
| 352697 | 69 | 0.70 | −1.05 | 1.42 | −1.64 | 3.06 | 0.0049 | SEQ ID NO: 1092 |
| 349052 | 70 | 0.19 | −0.15 | 9.59 | 0.71 | 2.69 | 0.0050 | SEQ ID NO: 1093 |
| 343517 | 69 | 0.41 | −0.67 | −3.32 | −1.05 | 2.72 | 0.0051 | SEQ ID NO: 1094 |
| 347425 | 69 | 0.57 | 0.33 | −4.65 | 0.13 | 3.11 | 0.0051 | SEQ ID NO: 1095 |
| 348867 | 69 | 0.18 | 1.15 | −1.82 | 2.47 | 3.06 | 0.0051 | SEQ ID NO: 1096 |
| 353212 | 67 | 0.60 | 0.74 | 1.59 | 0.98 | 2.82 | 0.0051 | SEQ ID NO: 1097 |
| 346115 | 70 | 0.17 | 1.65 | −0.42 | 2.11 | 2.93 | 0.0053 | SEQ ID NO: 1098 |
| 346232 | 70 | 0.69 | −1.07 | 1.03 | −1.49 | 2.76 | 0.0053 | SEQ ID NO: 1099 |
| 346976 | 70 | 0.81 | 0.15 | −9.59 | −0.71 | 2.69 | 0.0053 | SEQ ID NO: 1100 |
| 346890 | 70 | 0.51 | 0.64 | −1.58 | 0.62 | 2.80 | 0.0055 | SEQ ID NO: 1101 |
| 343386 | 70 | 0.14 | −0.96 | −2.04 | −2.35 | 3.16 | 0.0056 | SEQ ID NO: 1102 |
| 343526 | 70 | 0.02 | NaN | NaN | NaN | 3.02 | 0.0056 | SEQ ID NO: 1103 |
| 348771 | 70 | 0.74 | 0.95 | 1.21 | 1.48 | 2.98 | 0.0056 | SEQ ID NO: 1104 |
| 352285 | 70 | 0.26 | 1.07 | −1.09 | 1.64 | 2.86 | 0.0056 | SEQ ID NO: 1105 |
| 343589 | 69 | 0.74 | 0.81 | −0.88 | 0.47 | 2.72 | 0.0058 | SEQ ID NO: 1106 |
| 346225 | 67 | 0.17 | 0.03 | 47.70 | −1.05 | 2.76 | 0.0059 | SEQ ID NO: 1107 |
| 346963 | 70 | 0.23 | −0.28 | −6.95 | −1.33 | 2.77 | 0.0060 | SEQ ID NO: 1108 |
| 347235 | 70 | 0.84 | −1.73 | 0.58 | −2.41 | 2.68 | 0.0060 | SEQ ID NO: 1109 |
| 352932 | 70 | 0.76 | −0.22 | 8.03 | −1.10 | 2.82 | 0.0060 | SEQ ID NO: 1110 |
| 344987 | 70 | 0.94 | NaN | NaN | NaN | 2.68 | 0.0061 | SEQ ID NO: 1111 |
| 345162 | 69 | 0.59 | 0.15 | −9.46 | −0.11 | 2.75 | 0.0062 | SEQ ID NO: 1112 |
| 353824 | 70 | 0.59 | −0.13 | 10.94 | −0.37 | 2.67 | 0.0062 | SEQ ID NO: 1113 |
| 342514 | 68 | 0.28 | 0.62 | −2.63 | 1.35 | 2.60 | 0.0063 | SEQ ID NO: 1114 |
| 350192 | 70 | 0.20 | 1.01 | −1.89 | 2.16 | 3.02 | 0.0063 | SEQ ID NO: 1115 |
| 352518 | 70 | 0.92 | 0.04 | 59.62 | 1.99 | 2.72 | 0.0063 | SEQ ID NO: 1116 |
| 354161 | 70 | 0.73 | 0.71 | 2.11 | 1.40 | 2.79 | 0.0063 | SEQ ID NO: 1117 |
| 344034 | 56 | 0.25 | −0.37 | −6.12 | −1.51 | 3.09 | 0.0065 | SEQ ID NO: 1118 |
| 343548 | 69 | 0.04 | −0.25 | −14.10 | −3.40 | 2.94 | 0.0067 | SEQ ID NO: 1119 |
| 346163 | 69 | 0.14 | 0.36 | −6.44 | 2.01 | 3.24 | 0.0068 | SEQ ID NO: 1120 |
| 347731 | 69 | 0.07 | −0.03 | −85.07 | −2.29 | 2.82 | 0.0068 | SEQ ID NO: 1121 |
| 347988 | 69 | 0.57 | 0.50 | −2.39 | 0.34 | 2.72 | 0.0068 | SEQ ID NO: 1122 |
| 346563 | 70 | 0.87 | −1.77 | 0.87 | −2.92 | 2.63 | 0.0069 | SEQ ID NO: 1123 |
| 349526 | 69 | 0.22 | 0.54 | −2.60 | 1.30 | 2.92 | 0.0070 | SEQ ID NO: 1124 |
| 350110 | 70 | 0.84 | −1.07 | 1.81 | −2.36 | 2.87 | 0.0071 | SEQ ID NO: 1125 |
| 346359 | 70 | 0.16 | 1.73 | −0.58 | 2.41 | 2.68 | 0.0072 | SEQ ID NO: 1126 |
| 344197 | 70 | 0.89 | 1.31 | 2.42 | 3.75 | 2.96 | 0.0073 | SEQ ID NO: 1127 |
| 345543 | 70 | 0.33 | −0.36 | 3.18 | 0.03 | 2.66 | 0.0073 | SEQ ID NO: 1128 |
| 348364 | 70 | 0.31 | 0.93 | −1.90 | 1.58 | 2.86 | 0.0073 | SEQ ID NO: 1129 |
| 349329 | 70 | 0.62 | 0.78 | 1.46 | 1.05 | 2.82 | 0.0073 | SEQ ID NO: 1130 |
| 352030 | 70 | 0.43 | 0.09 | 16.66 | −0.12 | 2.72 | 0.0073 | SEQ ID NO: 1131 |
| 349054 | 70 | 0.20 | 0.09 | 16.38 | −0.77 | 2.73 | 0.0075 | SEQ ID NO: 1132 |
| 346097 | 70 | 0.36 | −0.16 | 8.47 | 0.21 | 2.77 | 0.0076 | SEQ ID NO: 1133 |
| 347411 | 69 | 0.62 | −0.28 | −4.96 | 0.04 | 2.82 | 0.0076 | SEQ ID NO: 1134 |
| 344199 | 67 | 0.51 | −0.78 | 1.41 | −0.81 | 2.76 | 0.0077 | SEQ ID NO: 1135 |
| 348199 | 70 | 0.17 | 0.33 | 3.75 | −0.48 | 2.69 | 0.0077 | SEQ ID NO: 1136 |
| 353569 | 69 | 0.38 | −0.78 | −1.45 | −1.06 | 2.76 | 0.0078 | SEQ ID NO: 1137 |
| 344596 | 67 | 0.54 | −0.21 | 5.83 | −0.32 | 2.76 | 0.0080 | SEQ ID NO: 1138 |
| 353284 | 68 | 0.77 | −0.81 | −0.89 | −0.42 | 2.77 | 0.0081 | SEQ ID NO: 1139 |
| 352909 | 69 | 0.60 | −0.01 | 276.76 | −0.31 | 2.62 | 0.0082 | SEQ ID NO: 1140 |
| 343004 | 70 | 0.69 | −1.22 | 0.94 | −1.64 | 2.73 | 0.0083 | SEQ ID NO: 1141 |
| 351453 | 70 | 0.74 | −1.35 | 1.28 | −2.18 | 2.62 | 0.0083 | SEQ ID NO: 1142 |
| 351946 | 70 | 0.26 | −0.58 | −3.06 | −1.42 | 2.68 | 0.0083 | SEQ ID NO: 1143 |
| 348729 | 70 | 0.79 | −1.22 | 1.51 | −2.26 | 2.72 | 0.0085 | SEQ ID NO: 1144 |
| 349694 | 70 | 0.19 | 0.85 | −2.37 | 2.10 | 2.68 | 0.0085 | SEQ ID NO: 1145 |
| 352290 | 70 | 0.32 | 1.37 | −0.76 | 1.75 | 2.87 | 0.0085 | SEQ ID NO: 1146 |
| 353571 | 70 | 0.11 | 1.82 | −1.17 | 3.47 | 2.90 | 0.0085 | SEQ ID NO: 1147 |
| 346774 | 70 | 0.34 | −0.47 | −2.52 | −0.84 | 2.74 | 0.0086 | SEQ ID NO: 1148 |
| 342952 | 70 | 0.12 | 1.49 | −1.52 | 3.21 | 2.41 | 0.0087 | SEQ ID NO: 1149 |
| 351900 | 70 | 0.21 | −0.65 | 1.29 | −0.16 | 2.73 | 0.0087 | SEQ ID NO: 1150 |
| 353979 | 68 | 0.22 | 0.75 | 0.85 | 0.39 | 2.68 | 0.0088 | SEQ ID NO: 1151 |
| 351500 | 65 | 0.58 | −0.49 | 2.86 | −0.72 | 2.54 | 0.0089 | SEQ ID NO: 1152 |
| 343483 | 68 | 0.86 | −0.83 | −0.95 | −0.26 | 2.66 | 0.0090 | SEQ ID NO: 1153 |
| 345229 | 69 | 0.52 | 0.46 | −2.40 | 0.42 | 2.70 | 0.0091 | SEQ ID NO: 1154 |
| 348702 | 70 | 0.57 | −0.83 | 1.40 | −1.00 | 2.76 | 0.0091 | SEQ ID NO: 1155 |
| 352790 | 70 | 0.07 | 0.64 | −2.53 | 2.03 | 2.92 | 0.0091 | SEQ ID NO: 1156 |
| 346071 | 69 | 0.38 | −0.33 | 3.87 | −0.02 | 2.66 | 0.0092 | SEQ ID NO: 1157 |
| 343443 | 69 | 0.90 | −0.39 | 6.14 | −2.28 | 2.46 | 0.0093 | SEQ ID NO: 1158 |
| 353654 | 66 | 0.58 | −0.92 | 1.32 | −1.13 | 2.85 | 0.0093 | SEQ ID NO: 1159 |
| 348109 | 68 | 0.54 | −0.50 | −2.29 | −0.40 | 2.67 | 0.0094 | SEQ ID NO: 1160 |
| 352690 | 70 | 0.27 | −0.58 | 1.42 | −0.20 | 2.60 | 0.0095 | SEQ ID NO: 1161 |
| 347812 | 65 | 0.65 | −0.32 | −3.91 | 0.05 | 2.56 | 0.0097 | SEQ ID NO: 1162 |
| 349320 | 70 | 0.71 | −0.85 | 1.57 | −1.40 | 2.70 | 0.0097 | SEQ ID NO: 1163 |
| 351125 | 70 | 0.76 | −0.87 | 2.03 | −1.80 | 2.96 | 0.0097 | SEQ ID NO: 1164 |
| 345061 | 70 | 0.89 | 0.86 | 3.08 | 2.92 | 2.65 | 0.0098 | SEQ ID NO: 1165 |
| 345972 | 70 | 0.04 | NaN | NaN | NaN | 2.60 | 0.0099 | SEQ ID NO: 1166 |
| 349045 | 70 | 0.89 | −1.27 | 1.09 | −2.33 | 2.23 | 0.0099 | SEQ ID NO: 1167 |
| 348554 | 70 | 0.89 | −0.94 | −1.30 | 0.02 | 2.64 | 0.0100 | SEQ ID NO: 1168 |
| 349093 | 68 | 0.32 | 0.39 | −3.00 | 0.80 | 2.59 | 0.0100 | SEQ ID NO: 1169 |

A further SNP (SEQ ID NO: 1170) with ident. 245097 located at SCAFFOLD 35329_4726 is also associated with RBY.

### EXAMPLE 8

An analysis of LDPF (an indicator of meat tenderness) was performed on the same animals and using the same method as previously. The associations identified are set forth in the following tables.

TABLE 28
Associations between DNA markers and meat tenderness sorted in decreasing order of
statistical significance. Locus is the ParAllele identifiers of the polymorphisms, N is
the number of genotypes, mean_0 is the mean meat tenderness for genotype 0, mean_1 is the
mean for genotype 1, and mean_2 is the mean of genotype 2, SD is the standard deviation, a
is the additive effect, k is the dominance effect, alpha is the average effect of allele
substitutions, tmax is the value of the t test, and log(1/P) is the P value determined from
100,000 permutation tests expressed as a positive integer. A minus value for a means the
mean of genotype 0 is lower than the mean of genotype 2, and a minus value for alpha means
that selecting for allele 0 will reduce the average values of tenderness in the population.

| Locus | mean_0 | SD | mean | SD | mean | SD | N | Freq | a | k | alpha | tmax | log(1/P) |
|---|---|---|---|---|---|---|---|---|---|---|---|---|---|
| 352458 | 0.00 | 0.00 | −0.29 | 0.60 | 0.23 | 0.65 | 163 | 0.22 | −0.12 | 3.52 | 0.11 | 7.465 | 5.0000 |
| 354432 | −0.14 | 0.53 | −0.10 | 0.74 | 0.53 | 0.64 | 167 | 0.63 | −0.34 | 0.88 | −0.41 | 7.254 | 4.6990 |
| 350589 | 0.12 | 0.67 | −0.06 | 0.69 | −0.54 | 0.41 | 168 | 0.74 | 0.33 | 0.45 | 0.40 | 7.530 | 4.6990 |
| 343164 | 0.09 | 0.73 | 0.04 | 0.52 | −0.46 | 0.37 | 165 | 0.73 | 0.28 | 0.82 | 0.38 | 7.037 | 4.5230 |
| 350373 | 0.20 | 0.70 | −0.08 | 0.61 | −0.35 | 0.54 | 165 | 0.62 | 0.28 | −0.02 | 0.27 | 6.458 | 4.3980 |
| 344369 | −0.05 | 0.58 | −0.26 | 0.59 | 0.29 | 0.78 | 166 | 0.56 | −0.17 | 2.24 | −0.22 | 6.090 | 4.3010 |
| 344304 | −0.08 | 0.72 | 0.25 | 0.58 | −0.27 | 0.47 | 166 | 0.65 | 0.10 | 4.47 | 0.22 | 6.464 | 4.0970 |
| 346793 | −0.21 | 0.68 | 0.12 | 0.64 | 0.40 | 0.52 | 169 | 0.67 | −0.30 | −0.08 | −0.30 | 6.625 | 3.9210 |
| 354280 | −0.43 | 0.45 | −0.07 | 0.63 | 0.17 | 0.71 | 164 | 0.33 | −0.30 | −0.20 | −0.32 | 6.814 | 3.8240 |
| 352917 | 0.02 | 0.66 | 0.13 | 0.70 | −0.43 | 0.49 | 168 | 0.66 | 0.23 | 1.49 | 0.33 | 6.346 | 3.8240 |
| 349256 | 0.03 | 0.61 | −0.27 | 0.61 | 0.24 | 0.67 | 162 | 0.50 | −0.10 | 3.86 | −0.10 | 5.954 | 3.8240 |
| 353554 | 0.18 | 0.68 | 0.00 | 0.68 | −0.34 | 0.52 | 169 | 0.58 | 0.26 | 0.31 | 0.27 | 6.118 | 3.7700 |
| 348551 | −0.19 | 0.58 | −0.04 | 0.72 | 0.33 | 0.62 | 169 | 0.55 | −0.26 | 0.42 | −0.27 | 5.889 | 3.7450 |
| 350739 | 0.50 | 0.55 | −0.38 | 0.67 | 0.01 | 0.65 | 168 | 0.14 | 0.24 | −2.59 | 0.71 | 6.931 | 3.6580 |
| 349717 | 0.26 | 0.73 | −0.13 | 0.64 | −0.23 | 0.51 | 167 | 0.56 | 0.24 | −0.59 | 0.23 | 5.875 | 3.6380 |
| 349493 | 0.39 | 0.63 | −0.03 | 0.68 | −0.15 | 0.64 | 166 | 0.41 | 0.27 | −0.56 | 0.30 | 5.796 | 3.6380 |
| 348201 | 0.02 | 0.65 | −0.38 | 0.67 | 0.50 | 0.55 | 166 | 0.86 | −0.24 | 2.67 | −0.70 | 6.926 | 3.5850 |
| 348641 | 0.00 | 0.00 | 0.34 | 0.54 | −0.09 | 0.68 | 169 | 0.10 | 0.04 | 8.56 | −0.26 | 5.362 | 3.5090 |
| 352071 | 0.01 | 0.65 | −0.38 | 0.67 | 0.53 | 0.57 | 166 | 0.87 | −0.26 | 2.50 | −0.74 | 6.789 | 3.4810 |
| 351532 | −0.01 | 0.65 | −0.14 | 0.73 | 0.39 | 0.45 | 169 | 0.68 | −0.20 | 1.65 | −0.32 | 6.100 | 3.4090 |
| 352862 | −0.21 | 0.68 | 0.12 | 0.64 | 0.37 | 0.52 | 168 | 0.68 | −0.29 | −0.14 | −0.28 | 6.250 | 3.3570 |
| 350492 | −0.22 | 0.63 | 0.04 | 0.56 | 0.29 | 0.73 | 164 | 0.59 | −0.26 | −0.02 | −0.25 | 5.641 | 3.3370 |
| 345126 | 0.00 | 0.00 | −0.48 | 0.49 | 0.04 | 0.67 | 167 | 0.06 | −0.02 | 25.00 | 0.42 | 5.679 | 3.3280 |
| 348301 | 0.01 | 0.65 | −0.27 | 0.69 | 0.46 | 0.58 | 169 | 0.80 | −0.23 | 2.24 | −0.53 | 6.361 | 3.3100 |
| 345252 | 0.25 | 0.72 | −0.13 | 0.65 | −0.21 | 0.51 | 169 | 0.56 | 0.23 | −0.65 | 0.21 | 5.538 | 3.3100 |
| 349269 | −0.22 | 0.63 | −0.06 | 0.70 | 0.26 | 0.61 | 169 | 0.48 | −0.24 | 0.33 | −0.24 | 5.531 | 3.2920 |
| 350161 | 0.20 | 0.73 | 0.05 | 0.64 | −0.27 | 0.54 | 167 | 0.50 | 0.24 | 0.36 | 0.24 | 5.428 | 3.2600 |
| 346399 | 0.37 | 0.64 | 0.03 | 0.63 | −0.16 | 0.67 | 168 | 0.37 | 0.27 | −0.28 | 0.29 | 5.738 | 3.2600 |
| 345633 | −0.08 | 0.65 | 0.04 | 0.71 | 0.63 | 0.42 | 168 | 0.81 | −0.35 | 0.66 | −0.50 | 6.748 | 3.2370 |
| 350748 | 0.55 | 0.64 | −0.17 | 0.66 | 0.01 | 0.63 | 164 | 0.23 | 0.27 | −1.67 | 0.51 | 6.210 | 3.2010 |
| 345991 | 0.08 | 0.69 | −0.30 | 0.52 | 0.00 | 0.00 | 168 | 0.89 | 0.04 | −8.50 | −0.23 | 4.994 | 3.1940 |
| 345122 | −0.08 | 0.65 | −0.11 | 0.71 | 0.40 | 0.56 | 169 | 0.66 | −0.24 | 1.12 | −0.32 | 5.583 | 3.1800 |
| 347902 | −0.02 | 0.70 | −0.02 | 0.36 | 0.71 | 0.08 | 148 | 0.93 | −0.36 | 1.00 | −0.68 | 10.732 | 3.1430 |
| 346374 | 0.05 | 0.74 | 0.08 | 0.61 | −0.41 | 0.49 | 167 | 0.66 | 0.23 | 1.13 | 0.32 | 5.676 | 3.1250 |
| 351296 | −0.05 | 0.65 | −0.05 | 0.67 | 0.76 | 0.58 | 165 | 0.82 | −0.41 | 1.00 | −0.66 | 6.533 | 3.1250 |
| 351108 | −0.26 | 0.53 | −0.03 | 0.64 | 0.19 | 0.73 | 165 | 0.44 | −0.23 | −0.02 | −0.23 | 5.318 | 3.1020 |
| 343239 | 0.41 | 0.70 | −0.16 | 0.70 | −0.05 | 0.59 | 168 | 0.32 | 0.23 | −1.48 | 0.35 | 5.626 | 3.0920 |
| 346822 | −0.10 | 0.69 | 0.05 | 0.57 | 0.53 | 0.59 | 169 | 0.79 | −0.32 | 0.52 | −0.41 | 5.946 | 3.0810 |
| 346774 | −0.47 | 0.43 | −0.08 | 0.70 | 0.09 | 0.66 | 167 | 0.24 | −0.28 | −0.39 | −0.34 | 6.067 | 3.0600 |
| 353386 | −0.04 | 0.73 | −0.02 | 0.49 | 0.62 | 0.45 | 168 | 0.81 | −0.33 | 0.94 | −0.52 | 6.491 | 3.0510 |
| 350817 | 0.28 | 0.73 | −0.15 | 0.67 | −0.17 | 0.49 | 161 | 0.54 | 0.23 | −0.91 | 0.21 | 5.249 | 3.0510 |
| 350047 | 0.17 | 0.73 | −0.04 | 0.64 | −0.31 | 0.52 | 167 | 0.62 | 0.24 | 0.12 | 0.25 | 5.441 | 3.0460 |
| 352010 | −0.30 | 0.58 | −0.09 | 0.69 | 0.18 | 0.65 | 169 | 0.36 | −0.24 | 0.12 | −0.23 | 5.406 | 3.0270 |
| 350345 | 0.51 | 0.32 | 0.03 | 0.67 | −0.07 | 0.69 | 167 | 0.20 | 0.29 | −0.66 | 0.40 | 6.433 | 3.0220 |
| 352120 | 0.31 | 0.56 | −0.15 | 0.68 | −0.05 | 0.68 | 169 | 0.46 | 0.18 | −1.56 | 0.20 | 5.276 | 3.0040 |
| 344748 | −0.37 | 0.49 | −0.02 | 0.61 | 0.15 | 0.74 | 159 | 0.36 | −0.26 | −0.35 | −0.29 | 5.682 | 3.0000 |
| 344204 | 0.27 | 0.66 | −0.01 | 0.78 | −0.19 | 0.50 | 167 | 0.46 | 0.23 | −0.22 | 0.23 | 5.165 | 2.9910 |
| 344057 | 0.32 | 0.60 | −0.18 | 0.73 | −0.01 | 0.62 | 168 | 0.36 | 0.17 | −2.03 | 0.26 | 5.399 | 2.9630 |
| 352035 | 0.31 | 0.71 | −0.08 | 0.61 | −0.16 | 0.64 | 166 | 0.43 | 0.23 | −0.66 | 0.26 | 5.224 | 2.9470 |
| 351814 | −0.37 | 0.88 | 1.51 | 0.29 | −0.03 | 0.62 | 150 | 0.08 | −0.17 | −10.06 | −1.62 | 9.825 | 2.9470 |
| 351799 | 0.04 | 0.68 | −0.02 | 0.63 | −0.58 | 0.49 | 134 | 0.73 | 0.31 | 0.81 | 0.43 | 5.899 | 2.9470 |
| 353810 | 0.35 | 0.61 | −0.38 | 0.45 | 0.00 | 0.68 | 164 | 0.13 | 0.17 | −3.17 | 0.58 | 6.020 | 2.9320 |
| 347485 | 0.23 | 0.69 | −0.13 | 0.64 | 0.00 | 0.00 | 167 | 0.68 | 0.12 | −2.13 | 0.03 | 4.644 | 2.9240 |
| 344670 | 0.01 | 0.68 | −0.20 | 0.64 | 0.25 | 0.61 | 168 | 0.56 | −0.12 | 2.75 | −0.16 | 5.136 | 2.8270 |
| 346326 | 0.00 | 0.62 | 0.16 | 0.63 | −0.34 | 0.75 | 168 | 0.61 | 0.17 | 1.94 | 0.24 | 5.159 | 2.8210 |
| 351177 | −0.03 | 0.64 | −0.10 | 0.76 | 0.60 | 0.48 | 169 | 0.82 | −0.32 | 1.22 | −0.56 | 6.066 | 2.8180 |
| 344652 | −0.40 | 0.57 | 0.05 | 0.61 | 0.08 | 0.72 | 167 | 0.32 | −0.24 | −0.88 | −0.32 | 5.273 | 2.8180 |
| 352015 | 0.03 | 0.74 | 0.09 | 0.62 | −0.33 | 0.37 | 168 | 0.71 | 0.18 | 1.33 | 0.28 | 5.302 | 2.8120 |
| 345053 | −0.05 | 0.56 | −0.22 | 0.61 | 0.35 | 0.87 | 167 | 0.69 | −0.20 | 1.85 | −0.34 | 5.246 | 2.7960 |
| 349726 | 0.39 | 0.57 | 0.06 | 0.68 | −0.13 | 0.66 | 169 | 0.28 | 0.26 | −0.27 | 0.29 | 5.369 | 2.7900 |
| 352030 | 0.07 | 0.61 | 0.18 | 0.70 | −0.26 | 0.63 | 169 | 0.46 | 0.17 | 1.67 | 0.14 | 5.021 | 2.7700 |
| 345729 | −0.17 | 0.54 | 0.04 | 0.75 | 0.36 | 0.74 | 169 | 0.66 | −0.27 | 0.21 | −0.28 | 5.150 | 2.7670 |
| 350569 | 0.65 | 0.49 | 0.00 | 0.65 | −0.05 | 0.67 | 169 | 0.15 | 0.35 | −0.86 | 0.56 | 6.053 | 2.7590 |
| 349322 | −0.40 | 0.46 | −0.12 | 0.67 | 0.14 | 0.67 | 169 | 0.27 | −0.27 | −0.04 | −0.27 | 5.719 | 2.7570 |
| 350828 | 0.09 | 0.71 | −0.06 | 0.66 | −0.36 | 0.39 | 167 | 0.73 | 0.22 | 0.33 | 0.26 | 5.333 | 2.7470 |
| 354133 | −0.75 | 0.10 | −0.07 | 0.64 | 0.07 | 0.66 | 166 | 0.16 | −0.41 | −0.66 | −0.59 | 11.233 | 2.7420 |
| 351908 | 0.59 | 0.44 | 0.00 | 0.69 | −0.08 | 0.66 | 144 | 0.19 | 0.33 | −0.76 | 0.49 | 6.016 | 2.7280 |
| 349414 | 0.04 | 0.71 | −0.18 | 0.57 | 0.41 | 0.69 | 168 | 0.68 | −0.18 | 2.19 | −0.33 | 5.417 | 2.7140 |
| 347372 | 0.02 | 0.73 | −0.24 | 0.49 | 0.27 | 0.42 | 152 | 0.79 | −0.12 | 3.08 | −0.35 | 5.556 | 2.7060 |
| 352630 | 0.10 | 0.71 | 0.00 | 0.00 | −0.22 | 0.55 | 169 | 0.68 | 0.16 | 0.38 | 0.18 | 4.474 | 2.7010 |
| 345533 | 0.48 | 0.43 | −0.09 | 0.71 | −0.05 | 0.67 | 153 | 0.21 | 0.27 | −1.15 | 0.44 | 5.375 | 2.6880 |
| 345385 | −0.03 | 0.73 | −0.16 | 0.65 | 0.27 | 0.53 | 167 | 0.61 | −0.15 | 1.87 | −0.21 | 4.999 | 2.6880 |
| 352676 | 0.25 | 0.72 | −0.15 | 0.64 | −0.19 | 0.51 | 169 | 0.61 | 0.22 | −0.82 | 0.18 | 5.096 | 2.6860 |
| 352031 | 0.00 | 0.00 | −0.43 | 0.58 | 0.05 | 0.67 | 168 | 0.06 | −0.03 | 18.20 | 0.38 | 4.772 | 2.6700 |
| 351329 | 0.34 | 0.72 | −0.04 | 0.60 | −0.15 | 0.69 | 168 | 0.41 | 0.24 | −0.55 | 0.27 | 4.980 | 2.6540 |
| 349451 | −0.13 | 0.76 | −0.05 | 0.61 | 0.33 | 0.59 | 169 | 0.58 | −0.23 | 0.65 | −0.25 | 4.978 | 2.6520 |
| 350012 | −0.19 | 0.51 | −0.14 | 0.64 | 0.25 | 0.72 | 168 | 0.39 | −0.22 | 0.77 | −0.18 | 5.081 | 2.6440 |
| 348539 | −0.64 | 0.05 | −0.37 | 0.51 | 0.07 | 0.68 | 169 | 0.08 | −0.35 | 0.24 | −0.28 | 9.664 | 2.6420 |
| 352883 | 0.23 | 0.71 | 0.00 | 0.62 | −0.20 | 0.64 | 169 | 0.47 | 0.22 | −0.07 | 0.22 | 4.816 | 2.6330 |
| 353110 | 0.34 | 0.52 | −0.15 | 0.68 | −0.07 | 0.66 | 163 | 0.29 | 0.21 | −1.39 | 0.33 | 5.139 | 2.6290 |
| 353540 | 0.03 | 0.72 | −0.18 | 0.51 | 0.62 | 0.62 | 169 | 0.79 | −0.29 | 1.71 | −0.59 | 6.080 | 2.6220 |
| 347776 | 0.29 | 0.66 | −0.02 | 0.68 | −0.16 | 0.63 | 168 | 0.44 | 0.22 | −0.38 | 0.24 | 4.928 | 2.6180 |
| 347337 | −0.04 | 0.58 | −0.15 | 0.68 | 0.30 | 0.73 | 168 | 0.58 | −0.17 | 1.65 | −0.21 | 4.874 | 2.6090 |
| 345183 | −0.16 | 0.62 | 0.04 | 0.67 | 0.33 | 0.72 | 169 | 0.66 | −0.24 | 0.18 | −0.26 | 4.967 | 2.5990 |
| 347847 | 0.24 | 0.75 | −0.13 | 0.64 | −0.18 | 0.44 | 169 | 0.59 | 0.21 | −0.76 | 0.18 | 4.985 | 2.5900 |
| 345239 | −0.10 | 0.57 | −0.15 | 0.73 | 0.28 | 0.67 | 168 | 0.54 | −0.19 | 1.26 | −0.21 | 4.764 | 2.5800 |
| 349959 | 0.08 | 0.70 | 0.00 | 0.00 | −0.24 | 0.56 | 169 | 0.73 | 0.16 | 0.50 | 0.20 | 4.407 | 2.5700 |
| 351886 | −0.16 | 0.62 | 0.09 | 0.65 | 0.33 | 0.72 | 167 | 0.66 | −0.24 | −0.02 | −0.24 | 4.970 | 2.5650 |
| 350949 | −0.09 | 0.62 | 0.05 | 0.78 | 0.39 | 0.26 | 168 | 0.77 | −0.24 | 0.42 | −0.29 | 5.670 | 2.5510 |
| 350428 | 0.19 | 0.69 | 0.00 | 0.00 | −0.13 | 0.64 | 164 | 0.43 | 0.16 | −0.19 | 0.16 | 4.273 | 2.5470 |
| 351887 | −0.19 | 0.59 | 0.00 | 0.65 | 0.26 | 0.74 | 169 | 0.57 | −0.23 | 0.16 | −0.23 | 4.834 | 2.5420 |
| 346186 | −0.25 | 0.55 | −0.14 | 0.62 | 0.18 | 0.71 | 169 | 0.34 | −0.21 | 0.49 | −0.18 | 4.940 | 2.5360 |
| 353562 | 0.25 | 0.66 | −0.20 | 0.66 | 0.02 | 0.67 | 160 | 0.42 | 0.12 | −2.91 | 0.17 | 4.776 | 2.5300 |
| 343331 | 0.21 | 0.73 | −0.13 | 0.56 | −0.26 | 0.65 | 155 | 0.61 | 0.23 | −0.45 | 0.21 | 4.880 | 2.5300 |
| 347985 | −0.07 | 0.64 | −0.05 | 0.68 | 0.54 | 0.66 | 169 | 0.73 | −0.31 | 0.93 | −0.44 | 5.290 | 2.5240 |
| 351430 | −0.16 | 0.57 | 0.06 | 0.74 | 0.38 | 0.62 | 169 | 0.67 | −0.27 | 0.19 | −0.29 | 5.251 | 2.5240 |
| 350070 | 0.49 | 0.67 | −0.11 | 0.58 | −0.04 | 0.69 | 167 | 0.23 | 0.27 | −1.26 | 0.44 | 5.307 | 2.5190 |
| 349171 | −0.10 | 0.66 | 0.03 | 0.72 | 0.37 | 0.35 | 168 | 0.70 | −0.23 | 0.45 | −0.28 | 5.541 | 2.5160 |
| 352063 | 0.38 | 0.62 | 0.07 | 0.75 | −0.16 | 0.57 | 168 | 0.33 | 0.27 | −0.15 | 0.28 | 5.234 | 2.5140 |
| 345458 | 0.38 | 0.62 | 0.06 | 0.78 | −0.17 | 0.57 | 159 | 0.32 | 0.28 | −0.16 | 0.29 | 5.176 | 2.5130 |
| 351280 | 0.28 | 0.70 | −0.16 | 0.74 | −0.10 | 0.57 | 162 | 0.44 | 0.19 | −1.32 | 0.22 | 4.733 | 2.5090 |
| 347804 | 0.15 | 0.60 | 0.02 | 0.68 | −0.32 | 0.71 | 168 | 0.56 | 0.23 | 0.45 | 0.25 | 4.842 | 2.5060 |
| 349968 | −0.05 | 0.71 | 0.01 | 0.45 | 0.67 | 0.58 | 169 | 0.85 | −0.36 | 0.83 | −0.57 | 5.751 | 2.4950 |
| 345565 | 0.59 | 0.42 | −0.09 | 0.52 | −0.02 | 0.70 | 168 | 0.14 | 0.30 | −1.23 | 0.57 | 6.178 | 2.4890 |
| 350013 | −0.40 | 0.67 | −0.13 | 0.71 | 0.16 | 0.61 | 167 | 0.29 | −0.28 | 0.04 | −0.28 | 5.128 | 2.4810 |
| 349939 | 0.01 | 0.69 | −0.16 | 0.57 | 0.57 | 0.44 | 159 | 0.86 | −0.28 | 1.61 | −0.61 | 6.178 | 2.4810 |
| 348694 | −0.05 | 0.68 | 0.07 | 0.71 | 0.46 | 0.13 | 164 | 0.83 | −0.26 | 0.53 | −0.34 | 6.595 | 2.4740 |
| 346954 | −0.10 | 0.66 | −0.03 | 0.73 | 0.35 | 0.52 | 163 | 0.67 | −0.22 | 0.69 | −0.28 | 4.921 | 2.4710 |
| 346612 | −0.24 | 0.72 | −0.01 | 0.61 | 0.19 | 0.66 | 168 | 0.46 | −0.21 | −0.07 | −0.22 | 4.662 | 2.4660 |
| 351005 | −0.19 | 0.74 | 0.03 | 0.64 | 0.28 | 0.56 | 168 | 0.58 | −0.24 | 0.06 | −0.24 | 4.928 | 2.4630 |
| 354399 | −0.16 | 0.61 | −0.03 | 0.70 | 0.26 | 0.65 | 169 | 0.54 | −0.21 | 0.38 | −0.22 | 4.734 | 2.4610 |
| 351252 | 0.06 | 0.67 | −0.34 | 0.61 | 0.00 | 0.00 | 169 | 0.91 | 0.03 | −12.33 | −0.27 | 4.381 | 2.4530 |
| 344150 | −0.02 | 0.64 | −0.10 | 0.75 | 0.48 | 0.51 | 168 | 0.79 | −0.25 | 1.32 | −0.44 | 5.328 | 2.4520 |
| 343878 | 0.20 | 0.73 | −0.17 | 0.50 | −0.26 | 0.60 | 165 | 0.67 | 0.23 | −0.61 | 0.18 | 4.963 | 2.4510 |
| 353271 | 0.33 | 0.64 | −0.12 | 0.65 | −0.08 | 0.66 | 165 | 0.44 | 0.21 | −1.20 | 0.24 | 4.792 | 2.4510 |
| 352480 | −0.14 | 0.63 | 0.00 | 0.00 | 0.17 | 0.70 | 168 | 0.57 | −0.16 | 0.10 | −0.16 | 4.206 | 2.4420 |
| 344346 | −0.10 | 0.64 | 0.00 | 0.00 | 0.25 | 0.72 | 168 | 0.71 | −0.17 | 0.43 | −0.21 | 4.214 | 2.4410 |
| 345210 | 0.22 | 0.71 | −0.11 | 0.69 | −0.19 | 0.48 | 157 | 0.56 | 0.21 | −0.61 | 0.19 | 4.699 | 2.4390 |
| 345566 | −0.03 | 0.61 | 0.17 | 0.80 | −0.36 | 0.44 | 150 | 0.73 | 0.16 | 2.21 | 0.33 | 5.266 | 2.4330 |
| 353106 | −0.24 | 0.61 | 0.06 | 0.70 | 0.19 | 0.64 | 169 | 0.54 | −0.21 | −0.40 | −0.21 | 4.770 | 2.4280 |
| 353813 | 0.06 | 0.63 | −0.19 | 0.67 | 0.28 | 0.67 | 167 | 0.61 | −0.11 | 3.27 | −0.19 | 4.855 | 2.4270 |
| 352768 | 0.39 | 0.26 | 0.06 | 0.80 | −0.08 | 0.63 | 166 | 0.22 | 0.24 | −0.40 | 0.29 | 5.443 | 2.4260 |
| 353773 | −0.28 | 0.58 | 0.05 | 0.64 | 0.15 | 0.71 | 163 | 0.42 | −0.22 | −0.53 | −0.23 | 4.732 | 2.4210 |
| 354517 | 0.26 | 0.64 | −0.03 | 0.69 | −0.16 | 0.63 | 168 | 0.45 | 0.21 | −0.38 | 0.22 | 4.682 | 2.4170 |
| 349502 | 0.19 | 0.71 | −0.09 | 0.62 | −0.28 | 0.63 | 169 | 0.63 | 0.24 | −0.19 | 0.22 | 4.869 | 2.4150 |
| 348150 | −0.43 | 0.43 | −0.01 | 0.74 | 0.06 | 0.65 | 169 | 0.24 | −0.24 | −0.71 | −0.34 | 5.215 | 2.4130 |
| 345962 | −0.11 | 0.66 | 0.00 | 0.00 | 0.20 | 0.67 | 169 | 0.65 | −0.15 | 0.29 | −0.17 | 4.137 | 2.4110 |
| 347342 | −0.22 | 0.42 | −0.18 | 0.69 | 0.19 | 0.71 | 159 | 0.36 | −0.21 | 0.80 | −0.16 | 4.747 | 2.4100 |
| 351744 | 0.47 | 0.11 | 0.24 | 0.68 | −0.06 | 0.67 | 168 | 0.11 | 0.27 | 0.13 | 0.24 | 6.048 | 2.4080 |
| 346141 | −0.01 | 0.73 | 0.06 | 0.57 | −0.45 | 0.26 | 168 | 0.80 | 0.22 | 1.32 | 0.40 | 6.175 | 2.4050 |
| 352178 | −0.32 | 0.51 | −0.06 | 0.70 | 0.13 | 0.67 | 168 | 0.34 | −0.23 | −0.16 | −0.24 | 4.954 | 2.3980 |
| 350341 | 0.39 | 0.21 | −0.05 | 0.77 | −0.01 | 0.64 | 169 | 0.22 | 0.20 | −1.20 | 0.33 | 5.592 | 2.3900 |
| 354427 | 0.06 | 0.68 | −0.17 | 0.67 | 0.34 | 0.47 | 168 | 0.72 | −0.14 | 2.64 | −0.30 | 5.226 | 2.3820 |
| 349074 | 0.42 | 0.71 | −0.17 | 0.66 | −0.01 | 0.66 | 165 | 0.24 | 0.21 | −1.74 | 0.41 | 5.030 | 2.3770 |
| 354434 | 0.35 | 0.64 | −0.19 | 0.80 | −0.01 | 0.57 | 166 | 0.27 | 0.18 | −2.00 | 0.34 | 4.955 | 2.3650 |
| 354514 | 0.03 | 0.73 | −0.02 | 0.52 | −0.54 | 0.29 | 169 | 0.82 | 0.29 | 0.82 | 0.44 | 6.359 | 2.3570 |
| 346469 | −0.18 | 0.52 | −0.17 | 0.64 | 0.24 | 0.70 | 167 | 0.37 | −0.21 | 0.95 | −0.16 | 4.802 | 2.3540 |
| 351516 | 0.52 | 0.59 | −0.15 | 0.70 | −0.03 | 0.66 | 155 | 0.16 | 0.28 | −1.44 | 0.54 | 5.157 | 2.3480 |
| 353376 | −0.18 | 0.61 | −0.01 | 0.64 | 0.23 | 0.74 | 169 | 0.53 | −0.21 | 0.17 | −0.21 | 4.513 | 2.3450 |
| 349243 | 0.24 | 0.66 | −0.21 | 0.59 | 0.03 | 0.69 | 168 | 0.33 | 0.10 | −3.29 | 0.22 | 4.734 | 2.3410 |
| 351514 | 0.29 | 0.71 | −0.01 | 0.65 | −0.17 | 0.62 | 167 | 0.42 | 0.23 | −0.30 | 0.24 | 4.696 | 2.3340 |
| 351693 | 0.32 | 0.63 | −0.11 | 0.67 | −0.09 | 0.65 | 165 | 0.44 | 0.21 | −1.10 | 0.23 | 4.629 | 2.3330 |
| 347527 | 0.01 | 0.66 | 0.00 | 0.71 | −0.75 | 0.10 | 168 | 0.86 | 0.38 | 0.97 | 0.65 | 9.399 | 2.3330 |
| 344424 | −0.10 | 0.57 | −0.15 | 0.73 | 0.26 | 0.67 | 167 | 0.54 | −0.18 | 1.28 | −0.20 | 4.587 | 2.3330 |
| 354446 | 0.04 | 0.70 | −0.22 | 0.53 | 0.33 | 0.64 | 166 | 0.77 | −0.15 | 2.79 | −0.36 | 4.974 | 2.3280 |
| 352498 | −0.27 | 0.56 | −0.15 | 0.62 | 0.16 | 0.69 | 163 | 0.34 | −0.22 | 0.44 | −0.19 | 4.696 | 2.3240 |
| 352838 | 0.32 | 0.53 | 0.08 | 0.71 | −0.14 | 0.65 | 169 | 0.30 | 0.23 | −0.04 | 0.23 | 4.844 | 2.3220 |
| 351757 | −0.03 | 0.64 | −0.04 | 0.77 | 0.39 | 0.21 | 168 | 0.78 | −0.21 | 1.05 | −0.33 | 5.412 | 2.3080 |
| 348797 | 0.00 | 0.62 | −0.21 | 0.60 | 0.20 | 0.67 | 164 | 0.39 | −0.10 | 3.10 | −0.03 | 4.577 | 2.2960 |
| 350264 | 0.08 | 0.70 | −0.10 | 0.53 | −0.62 | 0.58 | 169 | 0.82 | 0.35 | 0.49 | 0.46 | 5.487 | 2.2920 |
| 349865 | 0.50 | 0.52 | 0.04 | 0.69 | −0.07 | 0.67 | 167 | 0.22 | 0.29 | −0.61 | 0.38 | 5.195 | 2.2920 |
| 351118 | 0.00 | 0.00 | −0.46 | 0.58 | 0.04 | 0.67 | 169 | 0.06 | −0.02 | 24.00 | 0.41 | 4.426 | 2.2910 |
| 343649 | −0.23 | 0.59 | −0.02 | 0.61 | 0.19 | 0.77 | 167 | 0.47 | −0.21 | −0.00 | −0.21 | 4.523 | 2.2870 |
| 348008 | 0.25 | 0.61 | −0.19 | 0.68 | 0.03 | 0.67 | 166 | 0.34 | 0.11 | −3.00 | 0.21 | 4.647 | 2.2740 |
| 350078 | −0.05 | 0.67 | −0.08 | 0.62 | 0.48 | 0.72 | 168 | 0.71 | −0.27 | 1.11 | −0.39 | 4.888 | 2.2680 |
| 347050 | 0.31 | 0.55 | −0.14 | 0.70 | −0.02 | 0.66 | 164 | 0.32 | 0.17 | −1.73 | 0.27 | 4.769 | 2.2580 |
| 348953 | −0.58 | 0.53 | 0.01 | 0.54 | 0.03 | 0.70 | 168 | 0.18 | −0.30 | −0.93 | −0.49 | 5.328 | 2.2570 |
| 342980 | −0.16 | 0.66 | −0.04 | 0.58 | 0.24 | 0.71 | 167 | 0.57 | −0.20 | 0.40 | −0.21 | 4.483 | 2.2560 |
| 349659 | −0.46 | 0.25 | −0.15 | 0.53 | 0.06 | 0.71 | 169 | 0.14 | −0.26 | −0.19 | −0.30 | 6.058 | 2.2540 |
| 354087 | −0.10 | 0.61 | −0.05 | 0.77 | 0.31 | 0.52 | 167 | 0.62 | −0.21 | 0.76 | −0.24 | 4.573 | 2.2520 |
| 342582 | −0.04 | 0.66 | 0.21 | 0.66 | −0.41 | 0.57 | 135 | 0.80 | 0.18 | 2.35 | 0.44 | 5.053 | 2.2520 |
| 347814 | 0.02 | 0.68 | −0.33 | 0.37 | 0.00 | 0.00 | 168 | 0.96 | 0.01 | −34.00 | −0.30 | 4.411 | 2.2510 |
| 347778 | 0.20 | 0.81 | −0.21 | 0.59 | 0.09 | 0.54 | 167 | 0.52 | 0.06 | −6.45 | 0.04 | 4.501 | 2.2450 |
| 344925 | 0.00 | 0.66 | 0.11 | 0.68 | −0.62 | 0.59 | 169 | 0.81 | 0.31 | 1.35 | 0.57 | 5.472 | 2.2450 |
| 351028 | −0.62 | 0.58 | −0.05 | 0.53 | 0.08 | 0.70 | 166 | 0.17 | −0.35 | −0.63 | −0.49 | 5.435 | 2.2440 |
| 349443 | −0.62 | 0.58 | −0.11 | 0.54 | 0.07 | 0.70 | 165 | 0.18 | −0.34 | −0.48 | −0.45 | 5.358 | 2.2440 |
| 343670 | 0.29 | 0.60 | −0.07 | 0.68 | −0.11 | 0.66 | 163 | 0.44 | 0.20 | −0.80 | 0.22 | 4.518 | 2.2420 |
| 344792 | 0.27 | 0.25 | −0.19 | 0.58 | 0.03 | 0.71 | 168 | 0.16 | 0.12 | −2.83 | 0.35 | 5.497 | 2.2410 |
| 349075 | 0.57 | 0.44 | −0.10 | 0.60 | −0.01 | 0.69 | 169 | 0.15 | 0.29 | −1.31 | 0.56 | 5.686 | 2.2250 |
| 350061 | 0.21 | 0.66 | −0.01 | 0.73 | −0.18 | 0.57 | 169 | 0.48 | 0.20 | −0.13 | 0.20 | 4.451 | 2.2240 |
| 343573 | −0.12 | 0.69 | −0.04 | 0.58 | 0.29 | 0.66 | 164 | 0.60 | −0.20 | 0.61 | −0.23 | 4.454 | 2.2180 |
| 348073 | 0.00 | 0.00 | 0.37 | 0.64 | −0.06 | 0.67 | 169 | 0.07 | 0.03 | 13.33 | −0.32 | 4.150 | 2.2160 |
| 351617 | −0.09 | 0.50 | −0.12 | 0.72 | 0.28 | 0.76 | 169 | 0.55 | −0.18 | 1.16 | −0.21 | 4.405 | 2.2100 |
| 343686 | −0.20 | 0.60 | 0.05 | 0.72 | 0.25 | 0.69 | 152 | 0.57 | −0.23 | −0.11 | −0.22 | 4.487 | 2.2080 |
| 347128 | 0.22 | 0.75 | −0.02 | 0.61 | −0.17 | 0.63 | 168 | 0.49 | 0.20 | −0.23 | 0.20 | 4.415 | 2.2060 |
| 345849 | 0.36 | 0.70 | −0.07 | 0.75 | −0.11 | 0.58 | 156 | 0.35 | 0.23 | −0.83 | 0.29 | 4.546 | 2.2030 |
| 348732 | 0.26 | 0.77 | −0.10 | 0.63 | −0.14 | 0.56 | 169 | 0.51 | 0.20 | −0.80 | 0.20 | 4.394 | 2.1970 |
| 344187 | 0.04 | 0.71 | −0.17 | 0.59 | 0.33 | 0.69 | 168 | 0.67 | −0.15 | 2.45 | −0.27 | 4.728 | 2.1940 |
| 353457 | −0.04 | 0.74 | −0.12 | 0.65 | 0.26 | 0.53 | 169 | 0.58 | −0.15 | 1.53 | −0.19 | 4.471 | 2.1930 |
| 348194 | 0.09 | 0.79 | 0.07 | 0.62 | −0.28 | 0.44 | 169 | 0.59 | 0.18 | 0.89 | 0.22 | 4.439 | 2.1920 |
| 348309 | 0.00 | 0.00 | −0.36 | 0.26 | 0.01 | 0.69 | 169 | 0.02 | −0.01 | 73.00 | 0.34 | 4.960 | 2.1910 |
| 342797 | −0.10 | 0.61 | −0.15 | 0.66 | 0.28 | 0.71 | 147 | 0.50 | −0.19 | 1.26 | −0.19 | 4.432 | 2.1880 |
| 347426 | −0.12 | 0.63 | 0.02 | 0.75 | 0.31 | 0.60 | 164 | 0.65 | −0.21 | 0.35 | −0.24 | 4.496 | 2.1870 |
| 348989 | −0.25 | 0.59 | 0.03 | 0.63 | 0.14 | 0.75 | 168 | 0.45 | −0.20 | −0.44 | −0.20 | 4.401 | 2.1820 |
| 345540 | 0.11 | 0.70 | −0.07 | 0.66 | −0.29 | 0.48 | 166 | 0.69 | 0.20 | 0.10 | 0.21 | 4.583 | 2.1820 |
| 351288 | −0.31 | 0.66 | 0.07 | 0.71 | 0.09 | 0.57 | 168 | 0.46 | −0.20 | −0.90 | −0.21 | 4.451 | 2.1800 |
| 350073 | −0.09 | 0.60 | −0.16 | 0.69 | 0.22 | 0.68 | 169 | 0.49 | −0.15 | 1.45 | −0.15 | 4.378 | 2.1780 |
| 349533 | −0.04 | 0.68 | −0.08 | 0.65 | 0.42 | 0.58 | 169 | 0.73 | −0.23 | 1.17 | −0.35 | 4.808 | 2.1740 |
| 347780 | 0.00 | 0.00 | −0.46 | 0.49 | 0.03 | 0.68 | 169 | 0.03 | −0.01 | 31.67 | 0.43 | 4.431 | 2.1730 |
| 353170 | 0.03 | 0.69 | −0.18 | 0.62 | 0.27 | 0.67 | 167 | 0.64 | −0.12 | 2.75 | −0.21 | 4.551 | 2.1700 |
| 346247 | 0.37 | 0.58 | −0.17 | 0.70 | −0.03 | 0.64 | 162 | 0.21 | 0.20 | −1.70 | 0.40 | 4.793 | 2.1690 |
| 353615 | 0.21 | 0.55 | 0.03 | 0.69 | −0.18 | 0.68 | 165 | 0.42 | 0.20 | 0.08 | 0.19 | 4.454 | 2.1660 |
| 349615 | 0.26 | 0.77 | −0.10 | 0.63 | −0.14 | 0.56 | 169 | 0.51 | 0.20 | −0.80 | 0.20 | 4.394 | 2.1660 |
| 349410 | 0.14 | 0.74 | −0.10 | 0.51 | −0.35 | 0.63 | 168 | 0.71 | 0.24 | 0.02 | 0.25 | 4.747 | 2.1600 |
| 348228 | 0.03 | 0.69 | −0.20 | 0.64 | 0.37 | 0.27 | 168 | 0.86 | −0.17 | 2.35 | −0.46 | 5.907 | 2.1570 |
| 349428 | −0.11 | 0.66 | 0.14 | 0.56 | 0.47 | 0.57 | 152 | 0.75 | −0.29 | 0.14 | −0.31 | 5.045 | 2.1560 |
| 348499 | 0.07 | 0.71 | 0.00 | 0.00 | −0.21 | 0.51 | 169 | 0.75 | 0.14 | 0.50 | 0.18 | 3.942 | 2.1540 |
| 348616 | 0.00 | 0.00 | 0.88 | 0.66 | −0.04 | 0.65 | 166 | 0.02 | 0.02 | 45.00 | −0.84 | 5.097 | 2.1500 |
| 344943 | −0.16 | 0.51 | 0.07 | 0.80 | 0.30 | 0.68 | 167 | 0.65 | −0.23 | −0.00 | −0.23 | 4.551 | 2.1500 |
| 353418 | −0.02 | 0.69 | −0.08 | 0.62 | 0.57 | 0.44 | 163 | 0.85 | −0.29 | 1.20 | −0.55 | 5.507 | 2.1460 |
| 350887 | 0.00 | 0.00 | −0.25 | 0.52 | 0.06 | 0.69 | 169 | 0.10 | −0.03 | 9.33 | 0.19 | 3.961 | 2.1460 |
| 352155 | 0.55 | 0.56 | 0.10 | 0.76 | −0.12 | 0.60 | 168 | 0.23 | 0.34 | −0.34 | 0.40 | 5.318 | 2.1440 |
| 350548 | 0.22 | 0.73 | −0.02 | 0.68 | −0.18 | 0.56 | 158 | 0.47 | 0.20 | −0.20 | 0.20 | 4.355 | 2.1420 |
| 343172 | −0.07 | 0.57 | 0.25 | 0.66 | −0.15 | 0.78 | 159 | 0.52 | 0.04 | 9.00 | 0.05 | 4.306 | 2.1400 |
| 344280 | −0.34 | 0.46 | −0.07 | 0.64 | 0.10 | 0.71 | 169 | 0.27 | −0.22 | −0.23 | −0.24 | 4.769 | 2.1390 |
| 347779 | 0.00 | 0.00 | −0.46 | 0.49 | 0.03 | 0.68 | 169 | 0.03 | −0.01 | 31.67 | 0.43 | 4.431 | 2.1380 |
| 351593 | 0.08 | 0.63 | −0.25 | 0.61 | 0.00 | 0.00 | 163 | 0.90 | 0.04 | −7.25 | −0.19 | 3.927 | 2.1370 |
| 352925 | 0.03 | 0.69 | −0.46 | 0.50 | −0.20 | 0.23 | 157 | 0.90 | 0.12 | −3.26 | −0.19 | 5.149 | 2.1370 |
| 350271 | −0.09 | 0.65 | −0.09 | 0.68 | 0.32 | 0.63 | 168 | 0.56 | −0.21 | 1.00 | −0.23 | 4.443 | 2.1350 |
| 354191 | −0.11 | 0.60 | −0.05 | 0.72 | 0.29 | 0.61 | 163 | 0.54 | −0.20 | 0.70 | −0.21 | 4.400 | 2.1310 |
| 353848 | −0.15 | 0.61 | 0.00 | 0.00 | 0.13 | 0.71 | 168 | 0.46 | −0.14 | −0.07 | −0.14 | 3.803 | 2.1280 |
| 349215 | 0.29 | 0.58 | −0.18 | 0.74 | 0.04 | 0.63 | 169 | 0.28 | 0.12 | −2.76 | 0.27 | 4.636 | 2.1270 |
| 347654 | −0.09 | 0.60 | −0.11 | 0.71 | 0.28 | 0.60 | 162 | 0.53 | −0.18 | 1.11 | −0.20 | 4.395 | 2.1270 |
| 351599 | 0.00 | 0.00 | −0.32 | 0.58 | 0.06 | 0.68 | 167 | 0.07 | −0.03 | 11.67 | 0.27 | 3.991 | 2.1260 |
| 348341 | 0.04 | 0.63 | −0.18 | 0.74 | 0.29 | 0.58 | 169 | 0.72 | −0.12 | 2.76 | −0.27 | 4.636 | 2.1230 |
| 351341 | −0.53 | 0.37 | 0.07 | 0.69 | −0.02 | 0.67 | 166 | 0.17 | −0.26 | −1.35 | −0.48 | 5.591 | 2.1190 |
| 348709 | 0.02 | 0.63 | 0.08 | 0.71 | −0.50 | 0.61 | 168 | 0.75 | 0.26 | 1.23 | 0.42 | 4.957 | 2.1190 |
| 345282 | 0.40 | 0.79 | 0.14 | 0.63 | −0.16 | 0.62 | 166 | 0.25 | 0.28 | 0.07 | 0.27 | 4.638 | 2.1190 |
| 350712 | 0.09 | 0.69 | −0.10 | 0.56 | −0.53 | 0.65 | 164 | 0.81 | 0.31 | 0.39 | 0.38 | 4.953 | 2.1170 |
| 347470 | −0.02 | 0.59 | −0.17 | 0.66 | 0.22 | 0.70 | 168 | 0.43 | −0.12 | 2.25 | −0.08 | 4.381 | 2.1170 |
| 344137 | −0.25 | 0.53 | −0.01 | 0.70 | 0.14 | 0.70 | 162 | 0.38 | −0.20 | −0.23 | −0.21 | 4.371 | 2.1160 |
| 351472 | −0.03 | 0.62 | −0.14 | 0.79 | 0.97 | 0.47 | 161 | 0.88 | −0.50 | 1.22 | −0.97 | 6.858 | 2.1150 |
| 351594 | 0.11 | 0.68 | 0.04 | 0.69 | −0.27 | 0.58 | 169 | 0.57 | 0.19 | 0.63 | 0.21 | 4.380 | 2.1140 |
| 348558 | 0.38 | 0.78 | 0.00 | 0.70 | −0.13 | 0.59 | 167 | 0.31 | 0.26 | −0.49 | 0.30 | 4.527 | 2.1140 |
| 344829 | 0.24 | 0.74 | −0.14 | 0.69 | −0.08 | 0.53 | 169 | 0.49 | 0.16 | −1.38 | 0.17 | 4.302 | 2.1120 |
| 344892 | 0.00 | 0.00 | 0.42 | 0.53 | −0.03 | 0.68 | 169 | 0.05 | 0.01 | 29.00 | −0.37 | 4.177 | 2.1060 |
| 348849 | −0.40 | 0.56 | 0.02 | 0.69 | 0.07 | 0.66 | 169 | 0.34 | −0.24 | −0.79 | −0.29 | 4.722 | 2.1050 |
| 348785 | −0.26 | 0.54 | −0.07 | 0.50 | 0.13 | 0.75 | 155 | 0.33 | −0.20 | 0.03 | −0.19 | 4.471 | 2.1030 |
| 344060 | −0.07 | 0.66 | −0.06 | 0.64 | 0.43 | 0.70 | 169 | 0.68 | −0.25 | 0.96 | −0.34 | 4.615 | 2.0990 |
| 353163 | −0.33 | 0.49 | 0.00 | 0.60 | 0.07 | 0.74 | 168 | 0.31 | −0.20 | −0.65 | −0.25 | 4.605 | 2.0970 |
| 349697 | 0.00 | 0.00 | −0.24 | 0.65 | 0.08 | 0.69 | 140 | 0.17 | −0.04 | 7.00 | 0.15 | 3.819 | 2.0970 |
| 344779 | 0.04 | 0.63 | −0.18 | 0.74 | 0.29 | 0.58 | 169 | 0.72 | −0.12 | 2.76 | −0.27 | 4.636 | 2.0960 |
| 342961 | −0.18 | 0.61 | 0.04 | 0.63 | 0.20 | 0.67 | 155 | 0.49 | −0.19 | −0.16 | −0.19 | 4.286 | 2.0930 |
| 349664 | 0.21 | 0.72 | 0.13 | 0.72 | −0.20 | 0.57 | 169 | 0.38 | 0.21 | 0.61 | 0.18 | 4.371 | 2.0920 |
| 349589 | 0.02 | 0.67 | −0.46 | 0.49 | 0.00 | 0.00 | 168 | 0.97 | 0.01 | −47.00 | −0.43 | 4.352 | 2.0870 |
| 352802 | −0.18 | 0.50 | −0.14 | 0.74 | 0.19 | 0.64 | 168 | 0.38 | −0.18 | 0.78 | −0.15 | 4.386 | 2.0870 |
| 351515 | 0.27 | 0.66 | 0.06 | 0.65 | −0.17 | 0.68 | 166 | 0.38 | 0.22 | 0.05 | 0.22 | 4.431 | 2.0820 |
| 349971 | −0.58 | 0.53 | −0.15 | 0.64 | 0.09 | 0.67 | 168 | 0.18 | −0.33 | −0.28 | −0.40 | 5.275 | 2.0810 |
| 348988 | 0.13 | 0.76 | 0.06 | 0.63 | −0.26 | 0.57 | 169 | 0.52 | 0.20 | 0.64 | 0.20 | 4.301 | 2.0800 |
| 344472 | 0.15 | 0.70 | 0.02 | 0.61 | −0.24 | 0.64 | 161 | 0.49 | 0.20 | 0.33 | 0.19 | 4.279 | 2.0790 |
| 350408 | 0.32 | 0.66 | −0.08 | 0.74 | −0.09 | 0.59 | 167 | 0.39 | 0.21 | −0.95 | 0.25 | 4.358 | 2.0780 |
| 349588 | 0.02 | 0.68 | −0.46 | 0.49 | 0.00 | 0.00 | 167 | 0.97 | 0.01 | −47.00 | −0.43 | 4.381 | 2.0780 |
| 352706 | 0.00 | 0.57 | −0.16 | 0.83 | 0.35 | 0.64 | 168 | 0.73 | −0.17 | 1.91 | −0.33 | 4.602 | 2.0770 |
| 350342 | 0.31 | 0.62 | −0.06 | 0.64 | −0.09 | 0.67 | 157 | 0.41 | 0.20 | −0.85 | 0.23 | 4.359 | 2.0730 |
| 350422 | 0.06 | 0.68 | −0.28 | 0.61 | 0.00 | 0.00 | 169 | 0.89 | 0.03 | −10.33 | −0.21 | 3.858 | 2.0730 |
| 346111 | 0.47 | 0.83 | −0.15 | 0.48 | −0.01 | 0.67 | 160 | 0.22 | 0.24 | −1.58 | 0.45 | 4.684 | 2.0730 |
| 350034 | −0.17 | 0.68 | 0.05 | 0.65 | 0.27 | 0.66 | 167 | 0.62 | −0.22 | −0.00 | −0.22 | 4.455 | 2.0700 |
| 354077 | 0.01 | 0.60 | −0.17 | 0.62 | 0.28 | 0.84 | 168 | 0.62 | −0.14 | 2.33 | −0.21 | 4.344 | 2.0690 |
| 349115 | 0.09 | 0.67 | −0.16 | 0.65 | −0.58 | 0.53 | 167 | 0.83 | 0.33 | 0.25 | 0.39 | 5.262 | 2.0680 |
| 348784 | 0.04 | 0.68 | −0.28 | 0.55 | 0.00 | 0.00 | 166 | 0.91 | 0.02 | −15.00 | −0.23 | 3.891 | 2.0670 |
| 343676 | −0.07 | 0.70 | −0.20 | 0.56 | 0.22 | 0.69 | 165 | 0.40 | −0.15 | 1.90 | −0.09 | 4.370 | 2.0650 |
| 344893 | 0.18 | 0.74 | −0.20 | 0.64 | 0.06 | 0.61 | 168 | 0.50 | 0.06 | −5.33 | 0.06 | 4.241 | 2.0640 |
| 348659 | 0.16 | 0.73 | −0.22 | 0.55 | −0.01 | 0.68 | 165 | 0.57 | 0.09 | −3.47 | 0.04 | 4.276 | 2.0620 |
| 348473 | −0.02 | 0.67 | −0.17 | 0.65 | 0.22 | 0.67 | 169 | 0.44 | −0.12 | 2.25 | −0.09 | 4.308 | 2.0570 |
| 347210 | 0.01 | 0.67 | −0.64 | 0.32 | 0.00 | 0.00 | 167 | 0.99 | 0.01 | −129.00 | −0.62 | 6.016 | 2.0550 |
| 353336 | 0.10 | 0.40 | −0.32 | 0.52 | 0.10 | 0.72 | 169 | 0.20 | 0.00 | −420.00 | 0.00 | 4.797 | 2.0530 |
| 343893 | 0.54 | 0.16 | 0.00 | 0.73 | −0.04 | 0.64 | 168 | 0.17 | 0.29 | −0.86 | 0.45 | 7.069 | 2.0460 |
| 347145 | −0.03 | 0.67 | 0.68 | 0.44 | 0.00 | 0.00 | 168 | 0.98 | −0.01 | −46.33 | 0.66 | 5.361 | 2.0450 |
| 348396 | −0.67 | 0.42 | −0.08 | 0.62 | 0.13 | 0.71 | 169 | 0.29 | −0.40 | −0.47 | −0.48 | 6.420 | 2.0380 |
| 346918 | 0.14 | 0.73 | −0.14 | 0.58 | −0.24 | 0.54 | 162 | 0.67 | 0.19 | −0.47 | 0.16 | 4.337 | 2.0370 |
| 348189 | 0.30 | 0.67 | −0.02 | 0.73 | −0.12 | 0.56 | 166 | 0.41 | 0.21 | −0.52 | 0.23 | 4.371 | 2.0370 |
| 346600 | −0.42 | 0.55 | −0.10 | 0.70 | 0.15 | 0.65 | 159 | 0.26 | −0.28 | −0.12 | −0.30 | 4.918 | 2.0340 |
| 348351 | −0.54 | 0.72 | −0.12 | 0.57 | 0.10 | 0.68 | 169 | 0.21 | −0.32 | −0.31 | −0.38 | 4.823 | 2.0330 |
| 346946 | 0.27 | 0.56 | −0.12 | 0.60 | −0.04 | 0.80 | 167 | 0.46 | 0.15 | −1.52 | 0.17 | 4.235 | 2.0330 |
| 354486 | 0.30 | 0.53 | −0.06 | 0.77 | −0.08 | 0.62 | 168 | 0.37 | 0.19 | −0.89 | 0.23 | 4.357 | 2.0280 |
| 345732 | 0.44 | 0.32 | −0.20 | 0.60 | 0.04 | 0.70 | 167 | 0.14 | 0.20 | −2.20 | 0.51 | 6.007 | 2.0250 |
| 352955 | 0.32 | 0.67 | −0.05 | 0.62 | −0.10 | 0.69 | 168 | 0.36 | 0.21 | −0.76 | 0.25 | 4.336 | 2.0250 |
| 349733 | 0.23 | 0.78 | 0.05 | 0.69 | −0.19 | 0.54 | 169 | 0.42 | 0.21 | 0.14 | 0.21 | 4.330 | 2.0250 |
| 348340 | 0.04 | 0.66 | −0.18 | 0.71 | 0.28 | 0.54 | 169 | 0.73 | −0.12 | 2.83 | −0.28 | 4.595 | 2.0240 |
| 349894 | 0.40 | 0.63 | −0.11 | 0.68 | 0.00 | 0.66 | 168 | 0.28 | 0.20 | −1.55 | 0.33 | 4.632 | 2.0230 |
| 344077 | 0.24 | 0.73 | −0.15 | 0.58 | −0.05 | 0.69 | 169 | 0.52 | 0.14 | −1.69 | 0.14 | 4.194 | 2.0210 |
| 347673 | −0.30 | 0.50 | −0.02 | 0.68 | 0.10 | 0.69 | 168 | 0.32 | −0.20 | −0.40 | −0.23 | 4.428 | 2.0200 |
| 350805 | 0.30 | 0.64 | −0.04 | 0.64 | −0.12 | 0.61 | 161 | 0.37 | 0.21 | −0.62 | 0.24 | 4.363 | 2.0180 |
| 354192 | 0.25 | 0.56 | −0.07 | 0.74 | −0.11 | 0.59 | 166 | 0.47 | 0.18 | −0.78 | 0.19 | 4.272 | 2.0150 |
| 348178 | −0.01 | 0.70 | 0.10 | 0.58 | −0.66 | 0.42 | 169 | 0.86 | 0.33 | 1.34 | 0.64 | 5.966 | 2.0140 |
| 352353 | −0.02 | 0.64 | −0.04 | 0.77 | 0.37 | 0.20 | 168 | 0.79 | −0.20 | 1.10 | −0.32 | 5.069 | 2.0130 |
| 345638 | 0.11 | 0.72 | 0.06 | 0.66 | −0.26 | 0.56 | 168 | 0.58 | 0.18 | 0.73 | 0.21 | 4.238 | 2.0110 |
| 353486 | −0.10 | 0.58 | −0.02 | 0.71 | 0.40 | 0.70 | 167 | 0.65 | −0.25 | 0.68 | −0.30 | 4.534 | 2.0090 |
| 350079 | 0.06 | 0.71 | −0.15 | 0.54 | −0.61 | 0.27 | 169 | 0.86 | 0.33 | 0.37 | 0.42 | 6.813 | 2.0040 |
| 351774 | 0.06 | 0.71 | 0.03 | 0.61 | −0.39 | 0.62 | 169 | 0.68 | 0.23 | 0.87 | 0.30 | 4.478 | 2.0020 |
| 353896 | −0.72 | 0.59 | −0.09 | 0.65 | 0.06 | 0.66 | 169 | 0.14 | −0.39 | −0.62 | −0.56 | 5.310 | 2.0010 |

TABLE 29
SNP with the highest probability of being associated with LDPF.

| Ident | scaffold | contig chr. | Comment | | SEQ ID NO: |
|---|---|---|---|---|---|
| 352458 | SCAFFOLD75897_13740 | contig29503 | Hsa13 and Hsa1 Hsa1:CLCA4 to CLCA3, | | 1171 |
| | | | Hsa13:ABCC4 | | |
| 354432 | SCAFFOLD201769_1248 | Contig183125 | no chr. | | 1172 |
| 350589 | IBISS4snp140 | | Hsa4 | HOP transcript 2 | 1173 |
| 343164 | IBISS4snp311 | | Hsa1 | RPL11 | 1174 |
| 350373 | SCAFFOLD319464_327 | contig423442 | no chr. | | 1175 |
| 344369 | SCAFFOLD59697_1625 | contig329859 | Hsa12 | TULP3 intronl | 1176 |
| 344304 | SCAFFOLD323619_6623 | contig84765 | Hsa13 | TRPC4 | 1177 |
| 346793 | SCAFFOLD301627_1872 | contig682939 | Hsa10 | PARD3 | 1178 |
| 354280 | SCAFFOLD195092_4059 | contig52111 | Hsa6 | C6orf32 | 1179 |
| 352917 | SCAFFOLD46051_988 | contig126107 | Hsa18 | LOC390856 to MBD2 transcript1 | 1180 |

The idents are those in Table 28 and are the ParAllele identifiers. These are located on the BGSP scaffolds and contigs which indicate the SNP location. These scaffolds and contigs have been graphically located to maps on the biolives website.

TABLE 30
Parallele identifiers for further SNP associated with LDPF (meat tenderness)
showing the sequence scaffold, the Baylor College of Medicine sequence contig containing
the SNP, the Genbank Accession of the DNA sequence, and the alternative bases for each
SNP. The exact location of each SNP is shown in the sequence scaffold, the number after
the underscore is the exact base pair from the start of the scaffold.

| | | | | | | SEQ ID |
|---|---|---|---|---|---|---|
| ident | Ibiss4 | Sequence scaffold | Baylor Contig | Genbank Accession | SNP bases | NO |
| 342582 | Null | SCAFFOLD281406_2591 | Contig504531 | AAFC01504515 | A/G | 1181 |
| 342961 | Null | SCAFFOLD225832_5442 | Contig260139 | AAFC01260130 | A/G | 1182 |
| 342980 | Null | SCAFFOLD250434_1453 | Contig169120 | AAFC01169114 | C/T | 1183 |
| 343239 | Null | SCAFFOLD105231_4567 | Contig302225 | AAFC01302215 | C/T | 1184 |
| 343331 | Null | SCAFFOLD16085_17354 | Contig7459 | AAFC01007459 | A/T | 1185 |
| 343573 | Null | SCAFFOLD305385_2941 | Contig531782 | AAFC01531761 | C/T | 1186 |
| 343649 | Null | SCAFFOLD51430_8536 | Contig243793 | AAFC01243786 | C/T | 1187 |
| 343670 | Null | SCAFFOLD60719_4788 | Contig149503 | AAFC01149497 | A/G | 1188 |
| 343676 | Null | SCAFFOLD61161_13201 | Contig264651 | AAFC01264642 | A/G | 1189 |
| 343686 | Null | SCAFFOLD70038_5082 | Contig46463 | AAFC01046462 | A/G | 1190 |
| 343878 | Null | SCAFFOLD125630_2793 | Contig459251 | AAFC01459236 | C/G | 1191 |
| 343893 | Null | SCAFFOLD131120_4581 | Contig242406 | AAFC01242399 | G/T | 1192 |
| 344057 | Null | SCAFFOLD206025_18840 | Contig222205 | AAFC01222199 | G/T | 1193 |
| 344060 | Null | SCAFFOLD206561_7073 | Contig55526 | AAFC01055525 | C/T | 1194 |
| 344077 | Null | SCAFFOLD216310_4956 | Contig364044 | AAFC01364034 | C/T | 1195 |
| 344137 | Null | SCAFFOLD246051_2830 | Contig55665 | AAFC01055664 | C/T | 1196 |
| 344150 | Null | SCAFFOLD255245_11962 | Contig229301 | AAFC01229295 | C/T | 1197 |
| 344187 | Null | SCAFFOLD270085_5332 | Contig115177 | AAFC01115173 | G/T | 1198 |
| 344204 | Null | SCAFFOLD275958_4464 | Contig615262 | AAFC01615235 | G/T | 1199 |
| 344280 | Null | SCAFFOLD312466_13255 | Contig174869 | AAFC01174863 | A/G | 1200 |
| 344333 | Null | SCAFFOLD40347_458 | Contig718002 | AAFC01717966 | C/T | 1201 |
| 344346 | Null | SCAFFOLD4869_4182 | Contig139910 | AAFC01139905 | A/G | 1202 |
| 344424 | Null | SCAFFOLD85652_15416 | Contig400086 | AAFC01400073 | G/T | 1203 |
| 344652 | Null | SCAFFOLD150048_24207 | Contig643425 | AAFC01643397 | A/G | 1204 |
| 344670 | Null | SCAFFOLD155146_6104 | Contig422955 | AAFC01422942 | A/G | 1205 |
| 344748 | Null | SCAFFOLD195118_3818 | Contig423383 | AAFC01423370 | A/G | 1206 |
| 344779 | Null | SCAFFOLD205195_11503 | Contig40513 | AAFC01040512 | C/G | 1207 |
| 344792 | Null | SCAFFOLD210723_6215 | Contig7163 | AAFC01007163 | A/G | 1208 |
| 344829 | Null | SCAFFOLD222048_1096 | Contig389034 | AAFC01389022 | C/T | 1209 |
| 344892 | Null | SCAFFOLD245179_12372 | Contig18714 | AAFC01018714 | A/G | 1210 |
| 344893 | Null | SCAFFOLD245179_12153 | Contig18714 | AAFC01018714 | C/T | 1211 |
| 344925 | Null | SCAFFOLD251989_7150 | Contig337247 | AAFC01337237 | C/T | 1212 |
| 344943 | Null | SCAFFOLD26122_11530 | Contig318265 | AAFC01318255 | C/T | 1213 |
| 345053 | Null | SCAFFOLD305765_6934 | Contig54309 | AAFC01054308 | C/T | 1214 |
| 345122 | Null | SCAFFOLD45894_1473 | Contig53062 | AAFC01053061 | A/G | 1215 |
| 345126 | Null | SCAFFOLD50281_6253 | Contig12485 | AAFC01012485 | A/G | 1216 |
| 345183 | Null | SCAFFOLD70038_6516 | Contig267483 | AAFC01267474 | A/G | 1217 |
| 345210 | Null | SCAFFOLD76733_4424 | Contig153419 | AAFC01153413 | C/T | 1218 |
| 345239 | Null | SCAFFOLD85652_15587 | Contig400086 | AAFC01400073 | C/G | 1219 |
| 345252 | Null | SCAFFOLD90222_4388 | Contig57410 | AAFC01057409 | C/T | 1220 |
| 345385 | Null | SCAFFOLD11677_746 | Contig90789 | AAFC01090787 | C/T | 1221 |
| 345458 | Null | SCAFFOLD145703_5662 | Contig294239 | AAFC01294229 | A/G | 1222 |
| 345533 | Null | SCAFFOLD170036_23917 | Contig433770 | AAFC01433756 | C/T | 1223 |
| 345540 | Null | SCAFFOLD170731_17792 | Contig181621 | AAFC01181615 | A/G | 1224 |
| 345565 | Null | SCAFFOLD17903_1783 | Contig307879 | AAFC01307869 | A/G | 1225 |
| 345566 | Null | SCAFFOLD179698_13645 | Contig30125 | AAFC01030125 | A/G | 1226 |
| 345633 | Null | SCAFFOLD20938_6189 | Contig372277 | AAFC01372267 | A/G | 1227 |
| 345638 | Null | SCAFFOLD210185_14270 | Contig691429 | AAFC01691396 | A/G | 1228 |
| 345729 | Null | SCAFFOLD240110_24408 | Contig482987 | AAFC01482971 | A/C | 1229 |
| 345732 | Null | SCAFFOLD240396_5241 | Contig140894 | AAFC01140889 | C/G | 1230 |
| 345849 | Null | SCAFFOLD275605_2672 | Contig307051 | AAFC01307041 | A/G | 1231 |
| 345962 | Null | SCAFFOLD31854_1414 | Contig462699 | AAFC01462684 | G/T | 1232 |
| 345991 | Null | SCAFFOLD37811_702 | Contig273933 | AAFC01273924 | C/G | 1233 |
| 346111 | Null | SCAFFOLD75007_46300 | Contig33428 | AAFC01033427 | A/C | 1234 |
| 346141 | Null | SCAFFOLD86746_2591 | Contig12032 | AAFC01012032 | C/T | 1235 |
| 346247 | Null | SCAFFOLD10637_3753 | Contig143258 | AAFC01143253 | A/G | 1236 |
| 346326 | Null | SCAFFOLD141901_21478 | Contig34060 | AAFC01034059 | A/G | 1237 |
| 346374 | Null | SCAFFOLD160001_65148 | Contig263183 | AAFC01263174 | C/T | 1238 |
| 346399 | Null | SCAFFOLD165339_9908 | Contig111833 | AAFC01111830 | C/T | 1239 |
| 346469 | Null | SCAFFOLD185915_641 | Contig129957 | AAFC01129952 | A/G | 1240 |
| 346600 | Null | SCAFFOLD240058_6183 | Contig699663 | AAFC01699628 | A/C | 1241 |
| 346612 | Null | SCAFFOLD245097_12680 | Contig157834 | AAFC01157828 | C/T | 1242 |
| 346774 | Null | SCAFFOLD296060_5693 | Contig57805 | AAFC01057804 | A/G | 1243 |
| 346822 | Null | SCAFFOLD316847_12816 | Contig299928 | AAFC01299918 | C/T | 1244 |
| 346918 | Null | SCAFFOLD51516_2931 | Contig58742 | AAFC01058741 | C/T | 1245 |
| 346946 | Null | SCAFFOLD60719_8519 | Contig612068 | AAFC01612041 | C/G | 1246 |
| 346954 | Null | SCAFFOLD65896_3481 | Contig35855 | AAFC01035854 | C/T | 1247 |
| 347128 | Null | SCAFFOLD111035_12984 | Contig328357 | AAFC01328347 | C/T | 1248 |
| 347145 | Null | SCAFFOLD120223_9672 | Contig314303 | AAFC01314293 | C/G | 1249 |
| 347210 | Null | SCAFFOLD148828_1609 | Contig613701 | AAFC01613674 | A/G | 1250 |
| 347337 | Null | SCAFFOLD187095_554 | Contig635771 | AAFC01635743 | A/G | 1251 |
| 347342 | Null | SCAFFOLD190009_7530 | Contig28502 | AAFC01028502 | A/G | 1252 |
| 347372 | Null | SCAFFOLD20166_1744 | Contig98654 | AAFC01098652 | C/T | 1253 |
| 347426 | Null | SCAFFOLD216926_3322 | Contig351917 | AAFC01351907 | A/T | 1254 |
| 347470 | Null | SCAFFOLD231191_4220 | Contig279961 | AAFC01279952 | A/G | 1255 |
| 347485 | Null | SCAFFOLD240484_3069 | Contig74857 | AAFC01074856 | C/T | 1256 |
| 347527 | Null | SCAFFOLD25107_13912 | Contig640680 | AAFC01640652 | A/C | 1257 |
| 347654 | Null | SCAFFOLD291032_1968 | Contig416723 | AAFC01416710 | A/G | 1258 |
| 347673 | Null | SCAFFOLD296127_10747 | Contig423557 | AAFC01423544 | A/G | 1259 |
| 347776 | Null | SCAFFOLD36638_9971 | Contig148090 | AAFC01148084 | A/G | 1260 |
| 347778 | Null | SCAFFOLD37383_818 | Contig196420 | AAFC01196414 | C/T | 1261 |
| 347779 | Null | SCAFFOLD378810_561 | Contig448373 | AAFC01448359 | C/T | 1262 |
| 347804 | Null | SCAFFOLD46049_9345 | Contig161626 | AAFC01161620 | G/T | 1263 |
| 347814 | Null | SCAFFOLD50003_7693 | Contig263385 | AAFC01263376 | G/T | 1264 |
| 347847 | Null | SCAFFOLD60365_3678 | Contig608661 | AAFC01608634 | C/G | 1265 |
| 347902 | Null | SCAFFOLD75543_8475 | Contig43828 | AAFC01043827 | A/C | 1266 |
| 348073 | Null | SCAFFOLD110844_5365 | Contig41112 | AAFC01041111 | C/T | 1267 |
| 348150 | Null | SCAFFOLD135781_9137 | Contig555493 | AAFC01555471 | G/T | 1268 |
| 348178 | Null | SCAFFOLD14255_629 | Contig233552 | AAFC01233546 | C/T | 1269 |
| 348189 | Null | SCAFFOLD150363_3636 | Contig419701 | AAFC01419688 | A/G | 1270 |
| 348194 | Null | SCAFFOLD151_26514 | Contig212213 | AAFC01212207 | C/T | 1271 |
| 348201 | Null | SCAFFOLD152968_3124 | Contig433198 | AAFC01433184 | A/C | 1272 |
| 348228 | Null | SCAFFOLD160090_6137 | Contig26096 | AAFC01026096 | A/G | 1273 |
| 348301 | Null | SCAFFOLD195099_8219 | Contig30824 | AAFC01030824 | C/T | 1274 |
| 348309 | Null | SCAFFOLD196149_5586 | Contig46302 | AAFC01046301 | C/T | 1275 |
| 348340 | Null | SCAFFOLD205195_11413 | Contig40513 | AAFC01040512 | A/G | 1276 |
| 348341 | Null | SCAFFOLD205195_11304 | Contig40513 | AAFC01040512 | C/T | 1277 |
| 348351 | Null | SCAFFOLD20608_1008 | Contig661138 | AAFC01661110 | C/T | 1278 |
| 348396 | Null | SCAFFOLD22092_1589 | Contig292776 | AAFC01292766 | C/T | 1279 |
| 348473 | Null | SCAFFOLD251209_4281 | Contig226808 | AAFC01226802 | A/C | 1280 |
| 348499 | Null | SCAFFOLD260149_30694 | Contig339716 | AAFC01339706 | A/G | 1281 |
| 348539 | Null | SCAFFOLD270490_18616 | Contig241741 | AAFC01241734 | A/G | 1282 |
| 348551 | Null | SCAFFOLD275131_13295 | Contig64245 | AAFC01064244 | C/G | 1283 |
| 348558 | Null | SCAFFOLD275605_3060 | Contig307051 | AAFC01307041 | A/G | 1284 |
| 348616 | Null | SCAFFOLD300371_4264 | Contig109810 | AAFC01109807 | A/G | 1285 |
| 348641 | Null | SCAFFOLD310358_3310 | Contig327981 | AAFC01327971 | C/T | 1286 |
| 348659 | Null | SCAFFOLD316954_5829 | Contig232078 | AAFC01232072 | A/G | 1287 |
| 348694 | Null | SCAFFOLD36087_7085 | Contig582871 | AAFC01582847 | A/T | 1288 |
| 348709 | Null | SCAFFOLD40507_5003 | Contig53916 | AAFC01053915 | A/G | 1289 |
| 348732 | Null | SCAFFOLD50174_11362 | Contig23835 | AAFC01023835 | C/T | 1290 |
| 348784 | Null | SCAFFOLD65355_18740 | Contig145890 | AAFC01145884 | C/T | 1291 |
| 348785 | Null | SCAFFOLD65420_8328 | Contig107380 | AAFC01107377 | C/T | 1292 |
| 348797 | Null | SCAFFOLD70054_17340 | Contig359389 | AAFC01359379 | A/G | 1293 |
| 348849 | Null | SCAFFOLD90447_1749 | Contig248746 | AAFC01248738 | A/G | 1294 |
| 348953 | Null | SCAFFOLD115441_12359 | Contig732942 | AAFC01732903 | C/G | 1295 |
| 348988 | Null | SCAFFOLD130401_10619 | Contig120973 | AAFC01120969 | A/G | 1296 |
| 348989 | Null | SCAFFOLD130401_10348 | Contig120973 | AAFC01120969 | A/G | 1297 |
| 349074 | Null | SCAFFOLD15708_919 | Contig240205 | AAFC01240198 | A/G | 1298 |
| 349075 | Null | SCAFFOLD15708_1407 | Contig240205 | AAFC01240198 | C/T | 1299 |
| 349115 | Null | SCAFFOLD172970_4140 | Contig89859 | AAFC01089857 | C/T | 1300 |
| 349171 | Null | SCAFFOLD190769_7723 | Contig57695 | AAFC01057694 | C/T | 1301 |
| 349215 | Null | SCAFFOLD205195_11534 | Contig40513 | AAFC01040512 | G/T | 1302 |
| 349243 | Null | SCAFFOLD215128_19099 | Contig43014 | AAFC01043013 | A/T | 1303 |
| 349256 | Null | SCAFFOLD220358_6604 | Contig268451 | AAFC01268442 | A/G | 1304 |
| 349269 | Null | SCAFFOLD224445_646 | Contig688345 | AAFC01688313 | A/G | 1305 |
| 349322 | Null | SCAFFOLD238086_618 | Contig232761 | AAFC01232755 | A/C | 1306 |
| 349410 | Null | SCAFFOLD266863_5381 | Contig26960 | AAFC01026960 | A/C | 1307 |
| 349414 | Null | SCAFFOLD270085_5213 | Contig115177 | AAFC01115173 | A/G | 1308 |
| 349428 | Null | SCAFFOLD275080_6022 | Contig215992 | AAFC01215986 | A/C | 1309 |
| 349443 | Null | SCAFFOLD280379_15335 | Contig688136 | AAFC01688104 | C/T | 1310 |
| 349451 | Null | SCAFFOLD281769_3791 | Contig125907 | AAFC01125902 | C/T | 1311 |
| 349493 | Null | SCAFFOLD295848_7080 | Contig607587 | AAFC01607560 | A/G | 1312 |
| 349502 | Null | SCAFFOLD300370_3772 | Contig163296 | AAFC01163290 | C/T | 1313 |
| 349533 | Null | SCAFFOLD309057_1002 | Contig95744 | AAFC01095742 | C/T | 1314 |
| 349588 | Null | SCAFFOLD378810_660 | Contig448373 | AAFC01448359 | A/T | 1315 |
| 349615 | Null | SCAFFOLD50174_11664 | Contig23835 | AAFC01023835 | A/G | 1316 |
| 349659 | Null | SCAFFOLD6191_2381 | Contig191832 | AAFC01191826 | C/G | 1317 |
| 349664 | Null | SCAFFOLD65017_15757 | Contig751044 | AAFC01751000 | C/T | 1318 |
| 349697 | Null | SCAFFOLD80193_9585 | Contig510554 | AAFC01510536 | A/G | 1319 |
| 349717 | Null | SCAFFOLD90222_4848 | Contig57410 | AAFC01057409 | G/T | 1320 |
| 349726 | Null | SCAFFOLD91021_7030 | Contig65807 | AAFC01065806 | C/T | 1321 |
| 349733 | Null | SCAFFOLD93276_998 | Contig465924 | AAFC01465909 | A/G | 1322 |
| 349865 | Null | SCAFFOLD130500_11566 | Contig147653 | AAFC01147647 | C/T | 1323 |
| 349894 | Null | SCAFFOLD140160_3611 | Contig99310 | AAFC01099308 | A/G | 1324 |
| 349939 | Null | SCAFFOLD15708_1283 | Contig240205 | AAFC01240198 | G/T | 1325 |
| 349959 | Null | SCAFFOLD165938_4055 | Contig78906 | AAFC01078905 | G/T | 1326 |
| 349968 | Null | SCAFFOLD171671_5714 | Contig42823 | AAFC01042822 | A/T | 1327 |
| 349971 | Null | SCAFFOLD172970_4209 | Contig89859 | AAFC01089857 | C/T | 1328 |
| 350012 | Null | SCAFFOLD185915_514 | Contig129957 | AAFC01129952 | C/T | 1329 |
| 350013 | Null | SCAFFOLD186240_11795 | Contig7154 | AAFC01007154 | A/G | 1330 |
| 350034 | Null | SCAFFOLD193164_742 | Contig583609 | AAFC01583585 | G/T | 1331 |
| 350047 | Null | SCAFFOLD198293_737 | Contig405721 | AAFC01405708 | A/G | 1332 |
| 350061 | Null | SCAFFOLD205065_7733 | Contig650761 | AAFC01650733 | G/T | 1333 |
| 350070 | Null | SCAFFOLD205652_8347 | Contig119996 | AAFC01119992 | A/G | 1334 |
| 350073 | Null | SCAFFOLD205759_10331 | Contig79864 | AAFC01079863 | C/T | 1335 |
| 350078 | Null | SCAFFOLD206561_7143 | Contig55526 | AAFC01055525 | G/T | 1336 |
| 350079 | Null | SCAFFOLD207059_4048 | Contig67434 | AAFC01067433 | A/G | 1337 |
| 350161 | Null | SCAFFOLD245471_31606 | Contig452331 | AAFC01452317 | A/C | 1338 |
| 350264 | Null | SCAFFOLD280379_15411 | Contig688136 | AAFC01688104 | C/T | 1339 |
| 350271 | Null | SCAFFOLD280826_14575 | Contig43533 | AAFC01043532 | A/G | 1340 |
| 350341 | Null | SCAFFOLD310041_20415 | Contig39820 | AAFC01039819 | A/G | 1341 |
| 350342 | Null | SCAFFOLD310106_7051 | Contig250383 | AAFC01250375 | C/T | 1342 |
| 350345 | Null | SCAFFOLD311748_23096 | Contig185112 | AAFC01185106 | G/T | 1343 |
| 350408 | Null | SCAFFOLD36638_10144 | Contig148090 | AAFC01148084 | A/G | 1344 |
| 350422 | Null | SCAFFOLD45483_9360 | Contig200592 | AAFC01200586 | C/G | 1345 |
| 350428 | Null | SCAFFOLD4869_6549 | Contig139910 | AAFC01139905 | C/T | 1346 |
| 350492 | Null | SCAFFOLD70038_4486 | Contig46463 | AAFC01046462 | A/G | 1347 |
| 350548 | Null | SCAFFOLD85346_7303 | Contig118898 | AAFC01118894 | A/C | 1348 |
| 350569 | Null | SCAFFOLD94123_1052 | Contig235275 | AAFC01235268 | C/T | 1349 |
| 350712 | Null | SCAFFOLD141562_8222 | Contig18836 | AAFC01018836 | C/T | 1350 |
| 350739 | Null | SCAFFOLD152968_3192 | Contig433198 | AAFC01433184 | C/G | 1351 |
| 350748 | Null | SCAFFOLD15708_1291 | Contig240205 | AAFC01240198 | A/G | 1352 |
| 350805 | Null | SCAFFOLD180596_2419 | Contig403675 | AAFC01403662 | C/G | 1353 |
| 350817 | Null | SCAFFOLD185915_411 | Contig129957 | AAFC01129952 | A/G | 1354 |
| 350828 | Null | SCAFFOLD190280_13915 | Contig60243 | AAFC01060242 | C/T | 1355 |
| 350887 | Null | SCAFFOLD216607_24732 | Contig3170 | AAFC01003170 | A/C | 1356 |
| 350949 | Null | SCAFFOLD250404_9486 | Contig48271 | AAFC01048270 | C/T | 1357 |
| 351005 | Null | SCAFFOLD266863_5418 | Contig26960 | AAFC01026960 | C/T | 1358 |
| 351028 | Null | SCAFFOLD280379_15364 | Contig688136 | AAFC01688104 | C/T | 1359 |
| 351108 | Null | SCAFFOLD31598_11605 | Contig46911 | AAFC01046910 | A/G | 1360 |
| 351118 | Null | SCAFFOLD317761_5548 | Contig140235 | AAFC01140230 | A/G | 1361 |
| 351177 | Null | SCAFFOLD4277_4756 | Contig147836 | AAFC01147830 | C/T | 1362 |
| 351252 | Null | SCAFFOLD75157_12532 | Contig457294 | AAFC01457280 | A/G | 1363 |
| 351280 | Null | SCAFFOLD85652_15208 | Contig400086 | AAFC01400073 | A/C | 1364 |
| 351288 | Null | SCAFFOLD90097_20876 | Contig169910 | AAFC01169904 | A/G | 1365 |
| 351296 | Null | SCAFFOLD94123_1093 | Contig235275 | AAFC01235268 | A/C | 1366 |
| 351341 | Null | SCAFFOLD100068_6509 | Contig62523 | AAFC01062522 | A/G | 1367 |
| 351430 | Null | SCAFFOLD145703_5870 | Contig294239 | AAFC01294229 | C/G | 1368 |
| 351472 | Null | SCAFFOLD167798_5562 | Contig254281 | AAFC01254273 | G/T | 1369 |
| 351514 | Null | SCAFFOLD192666_11865 | Contig351258 | AAFC01351248 | C/T | 1370 |
| 351515 | Null | SCAFFOLD193164_514 | Contig583609 | AAFC01583585 | A/G | 1371 |
| 351516 | Null | SCAFFOLD195099_7892 | Contig30824 | AAFC01030824 | A/T | 1372 |
| 351532 | Null | SCAFFOLD200558_6470 | Contig20558 | AAFC01020558 | A/C | 1373 |
| 351593 | Null | SCAFFOLD234818_2292 | Contig117676 | AAFC01117672 | C/T | 1374 |
| 351594 | Null | SCAFFOLD235169_2062 | Contig552567 | AAFC01552545 | A/T | 1375 |
| 351599 | Null | SCAFFOLD239395_810 | Contig376366 | AAFC01376356 | A/C | 1376 |
| 351617 | Null | SCAFFOLD250354_2182 | Contig75684 | AAFC01075683 | C/T | 1377 |
| 351693 | Null | SCAFFOLD280826_14537 | Contig43533 | AAFC01043532 | A/T | 1378 |
| 351744 | Null | SCAFFOLD30172_28453 | Contig54331 | AAFC01054330 | A/G | 1379 |
| 351757 | Null | SCAFFOLD310041_20197 | Contig39820 | AAFC01039819 | C/T | 1380 |
| 351774 | Null | SCAFFOLD314614_649 | Contig391685 | AAFC01391673 | C/T | 1381 |
| 351799 | Null | SCAFFOLD324639_528 | Contig686196 | AAFC01686164 | A/G | 1382 |
| 351814 | Null | SCAFFOLD36570_1809 | Contig232548 | AAFC01232542 | A/G | 1383 |
| 351886 | Null | SCAFFOLD70038_4640 | Contig46463 | AAFC01046462 | C/T | 1384 |
| 351887 | Null | SCAFFOLD70038_7077 | Contig267483 | AAFC01267474 | A/G | 1385 |
| 351908 | Null | SCAFFOLD84744_294 | Contig138116 | AAFC01138111 | A/G | 1386 |
| 352010 | Null | SCAFFOLD120898_10475 | Contig344712 | AAFC01344702 | C/T | 1387 |
| 352015 | Null | SCAFFOLD121654_12895 | Contig33470 | AAFC01033469 | A/G | 1388 |
| 352030 | Null | SCAFFOLD125746_1703 | Contig242144 | AAFC01242137 | C/T | 1389 |
| 352031 | Null | SCAFFOLD125755_3155 | Contig543641 | AAFC01543620 | C/G | 1390 |
| 352035 | Null | SCAFFOLD130001_59457 | Contig249594 | AAFC01249586 | A/C | 1391 |
| 352063 | Null | SCAFFOLD145703_5705 | Contig294239 | AAFC01294229 | C/G | 1392 |
| 352071 | Null | SCAFFOLD152968_3080 | Contig433198 | AAFC01433184 | A/G | 1393 |
| 352120 | Null | SCAFFOLD176108_7456 | Contig143077 | AAFC01143072 | A/G | 1394 |
| 352155 | Null | SCAFFOLD200364_14961 | Contig268455 | AAFC01268446 | C/T | 1395 |
| 352178 | Null | SCAFFOLD210854_1158 | Contig387860 | AAFC01387848 | C/T | 1396 |
| 352353 | Null | SCAFFOLD310041_20086 | Contig39820 | AAFC01039819 | A/G | 1397 |
| 352480 | Null | SCAFFOLD86884_422 | Contig572598 | AAFC01572576 | C/G | 1398 |
| 352630 | Null | SCAFFOLD165938_4212 | Contig78906 | AAFC01078905 | A/G | 1399 |
| 352676 | Null | SCAFFOLD185915_226 | Contig129957 | AAFC01129952 | A/G | 1400 |
| 352706 | Null | SCAFFOLD20780_6570 | Contig448610 | AAFC01448596 | A/G | 1401 |
| 352768 | Null | SCAFFOLD250404_9928 | Contig48271 | AAFC01048270 | C/T | 1402 |
| 352802 | Null | SCAFFOLD270318_12392 | Contig333590 | AAFC01333580 | C/T | 1403 |
| 352838 | Null | SCAFFOLD291604_12415 | Contig38390 | AAFC01038389 | A/T | 1404 |
| 352862 | Null | SCAFFOLD301627_1720 | Contig682939 | AAFC01682907 | A/T | 1405 |
| 352883 | Null | SCAFFOLD313308_2790 | Contig451440 | AAFC01451426 | C/T | 1406 |
| 352925 | Null | SCAFFOLD50174_11343 | Contig23835 | AAFC01023835 | A/G | 1407 |
| 352955 | Null | SCAFFOLD68748_1948 | Contig508180 | AAFC01508164 | A/G | 1408 |
| 353106 | Null | SCAFFOLD155476_15944 | Contig79654 | AAFC01079653 | A/G | 1409 |
| 353110 | Null | SCAFFOLD158271_1999 | Contig369257 | AAFC01369247 | C/G | 1410 |
| 353163 | Null | SCAFFOLD198293_824 | Contig405721 | AAFC01405708 | A/T | 1411 |
| 353170 | Null | SCAFFOLD20021_17935 | Contig13105 | AAFC01013105 | C/G | 1412 |
| 353271 | Null | SCAFFOLD280826_14286 | Contig43533 | AAFC01043532 | A/G | 1413 |
| 353336 | Null | SCAFFOLD352562_535 | Contig94850 | AAFC01094848 | A/G | 1414 |
| 353376 | Null | SCAFFOLD68748_2379 | Contig508180 | AAFC01508164 | A/G | 1415 |
| 353386 | Null | SCAFFOLD76639_3861 | Contig334124 | AAFC01334114 | A/G | 1416 |
| 353457 | Null | SCAFFOLD125279_19173 | Contig29410 | AAFC01029410 | A/G | 1417 |
| 353486 | Null | SCAFFOLD147091_1735 | Contig108498 | AAFC01108495 | A/G | 1418 |
| 353540 | Null | SCAFFOLD20021_18293 | Contig13105 | AAFC01013105 | A/C | 1419 |
| 353554 | Null | SCAFFOLD221921_4981 | Contig258191 | AAFC01258182 | A/T | 1420 |
| 353562 | Null | SCAFFOLD235688_5521 | Contig353473 | AAFC01353463 | C/G | 1421 |
| 353615 | Null | SCAFFOLD285455_2244 | Contig237836 | AAFC01237829 | A/G | 1422 |
| 353773 | Null | SCAFFOLD150048_24355 | Contig643425 | AAFC01643397 | A/G | 1423 |
| 353810 | Null | SCAFFOLD195099_8276 | Contig30824 | AAFC01030824 | A/C | 1424 |
| 353813 | Null | SCAFFOLD20021_18112 | Contig13105 | AAFC01013105 | C/T | 1425 |
| 353848 | Null | SCAFFOLD260045_330 | Contig564187 | AAFC01564165 | C/T | 1426 |
| 353896 | Null | SCAFFOLD35672_327 | Contig490593 | AAFC01490577 | C/T | 1427 |
| 354077 | Null | SCAFFOLD40347_337 | Contig718002 | AAFC01717966 | G/T | 1428 |
| 354087 | Null | SCAFFOLD65896_3078 | Contig35855 | AAFC01035854 | A/G | 1429 |
| 354133 | Null | SCAFFOLD152083_9294 | Contig256770 | AAFC01256761 | C/T | 1430 |
| 354191 | Null | SCAFFOLD291032_1734 | Contig416723 | AAFC01416710 | A/T | 1431 |
| 354192 | Null | SCAFFOLD291032_1759 | Contig416723 | AAFC01416710 | A/G | 1432 |
| 354399 | Null | SCAFFOLD65896_3250 | Contig35855 | AAFC01035854 | A/G | 1433 |
| 354427 | Null | SCAFFOLD185006_42127 | Contig29275 | AAFC01029275 | C/G | 1434 |
| 354434 | Null | SCAFFOLD20780_8873 | Contig448610 | AAFC01448596 | A/G | 1435 |
| 354446 | Null | SCAFFOLD289241_10601 | Contig3644 | AAFC01003644 | A/C | 1436 |
| 354486 | Null | SCAFFOLD65896_3125 | Contig35855 | AAFC01035854 | A/C | 1437 |
| 354514 | Null | SCAFFOLD92868_1952 | Contig625847 | AAFC01625819 | A/G | 1438 |
| 354517 | Null | SCAFFOLD131415_1790 | Contig267180 | AAFC01267171 | A/G | 1439 |
| 354499 | Null | SCAFFOLD286022_2896 | Contig353540 | AAFC01353530 | A/C | 1440 |

TABLE 31

SNP associated with LDPF (meat tenderness)
obtained from the IBISS4 database.

| | |
|---|---|
| IBISS4snp154 IBISS4 btcn16717 7/13 404 | T/C |
| AGTGCAAGGCTGGGGGCGAGAGCCTGGCCCACTGGCTTTCATGCAACTTC | Homo sapiens insulin-like |
| GTGAGTCACGCACTGTGCAAATTAAGGCTTCTTATAGGTCGAATGGTCGA | growth factor 2 |
| | receptor (IGF2R), mRNA 3e−25 |
| | (SEQ ID NO:1441) |
| IBISS4snp266 IBISS4 btcn1969 10/19 458 | T/C |
| TGACAGTTAAACACATCTTTTAAAGCATAGACACACATAGAAAAAACATA | Homo sapiens PEST-containing |
| GGTATAATGATTTCCTTGAAGACTACAATGTTAGATTTGGAGGCAGCTTC | nuclear protein (PCNP), |
| | mRNA 1e−110 |
| | (SEQ ID NO:1442) |
| IBISS4snp297 IBISS4 btcn20629 5/11 1188 | C/T |
| CTTTAAGACATGGCTTACTTTACCTCACTATCAATGGAGGGAGAAAGGAA | Homo sapiens a disintegrin-like |
| GCACATGGGATCTTTGACCATCACTTTACCCGCTGCTATGGTTTCAGAGA | and metalloprotease (reprolysin |
| | type) with thrombospondin |
| | type 1 motif, 1 (ADAMTS1), |
| | mRNA 1e−162 |
| | (SEQ ID NO:1443) |
| IB1SS4snp298 IBISS4 btcn20629 6/11 1379 | A/G |
| CCTCTCTTGTGTCGTTATTATGTCTTTCTTTGTGAATTCCTTTTGACGAA | Homo sapiens a disintegrin-like |
| AAACAATTGCATGTATTTGTAAACACACCATAAATTCTACAAGGGGAAAA | and metalloprotease (reprolysin |
| | type) with thrombospondin |
| | type 1 motif, 1 (ADAMTS1), |
| | mRNA 1e−162 |
| | (SEQ ID NO:1444) |
| IBISS4snp343 IBISS4 btcn22113 7/12 108 | A/G |
| CAAGGAAGCTGAGTCCACTCAACTAGAAGAGAGCGAAGACA | Homo sapiens OCIA domain |
| AGGTCATCATGGCTTCAGTGTCCACACATGAAAACCAAGAA | containing 2 (OCIAD2), mRNA 1e−87 |
| | (SEQ ID NO:1445) |
| IBISS4snp371 IBISS4 btcn23O4 7/12 1623 | C/T |
| GATTAGTTTAACGAGACTTAACTGTTCCCACCTAAGACTGTCAGAGGGAG | Homo sapiens thioredoxin-like 1 |
| ACAGATTTGATACCCTTTTGGATAGACTTAAAACTTGAGTCATCTTTATC | (TXNL1), mRNA 0.0 |
| | (SEQ ID NO:1446) |
| IBISS4snp404 IBISS4 btcn23970 21/34 1271 | G/T |
| ACTTGATCAACTTAATTCCTTTTCTTTATCTTCCCTCCCTCACTCCCCTT | Homo sapiens actin related |
| CCTGCCCCTTCTTTTCCAAGCTGTTTTGCTTTGCAATATGTTACTGGTAA | protein 2/3 complex, subunit |
| | 2, 34kDa (ARPC2), transcript |
| | variant 1, mRNA 0.0 |
| | (SEQ ID NO:1447) |
| IBISS4snp483 IBISS4 btcn26603 18/31 851 | T/C |
| GAGTTCTTGAGCTTTGCCGAAAGGTACCCTACCATCGTCTATAACATCCT | Homo sapiens solute carrier |
| CTCTTTGGCCTGACTAGTGCCCTGGGCCAG | family 35, member B1 |
| | (SLC35B1), mRNA 0.0 |
| | (SEQ ID NO:1448) |
| IBISS4snp695 IBISS4 btcn34762 6/10 932 | G/A |
| TGACTGCCTTCTCTGCAGGTTGAAGCCCAGAGCTTTAATTATTCGTATGC | Homo sapiens aldehyde |
| GCTATGATAGATAGCATTTTACTTGAATAATATTTATGCTGTGGTTTTTT | dehydrogenase 5 family, member |
| | A1 (succinate-semialdehyde |
| | dehydrogenase) (ALDH5A1), |
| | nuclear gene encoding |
| | mitochondrial protein, |
| | transcript variant 1, mRNA 1e−06 |
| | (SEQ ID NO: 1449) |
| IBISS4snp941 IBISS4 btcn53153 7/12 573 | T/C |
| TATAATCCTCGAATCCAGGAGATCCAAGTTGTCAAATTAGAGAAACGGCT | Homo sapiens mitochondrial |
| GACGATAGTTTGCTGTACTTGCGAGATGCCCTTCCTGAATACAGCACTTT | ribosomal protein L19 (MRPL19), |
| | nuclear gene encoding |
| | mitochondrial protein, mRNA 0.0 |
| | (SEQ ID NO:1450) |
| IBISS4snp989 IBISS4 btcn6326 9/16 212 | T/C |
| CAGCTCCGGCTCTCAGCCTGTCTGCATCGCCCCCCTCCTCCCGGATCCTG | Homo sapiens creatine kinase, |
| CCTGAGCCAGCAGCTCCTGCCCTGAGATTCCGTAGGCAGACCCTCAAGCC | mitochondrial 1 (ubiquitous) (CKMT1), |
| | nuclear gene encoding mitochondrial |
| | protein, mRNA 0.0 |
| | (SEQ ID NO:1451) |

### EXAMPLE 9

To find additional loci affecting meat tenderness, further analyses of the whole genome SNP scan were performed. First, the LDPF trait values for all measured individuals, not just the 189 in the whole genome scan, were adjusted using the model ldpf ˜ mu herd kill_group age ! sireid using ASREML. The residual LDPF phenotypes were then available for further analysis. The means and standard errors of the residual LDPF for each genotype in the whole genome scan were calculated and at test computed for each comparison. The statistical significance of the largest mean difference was calculated using 100,000 permutations.

TABLE 32
A set of loci with high levels of support
associated with residual meat tenderness.

| | scaffold- | | | | | SEQ ID |
|---|---|---|---|---|---|---|
| Snp_id | v2 | bp | scaffold-v1 | Hsa | Gene | NO |
| 344371 | Un.2692 | 15944 | SCAFFOLD6014_7952 | 2 | no gene near | 1452 |
| 348385 | Un.3082 | 36364 | SCAFFOLD220007_32473 | 4 | no gene near | 1453 |
| 350387 | 25.18 | 276632 | SCAFFOLD325320_4318 | 16 | LOC283953 | 1454 |
| | | | | | expressed gene | |
| 354161 | Un.3082 | 36240 | SCAFFOLD220007_32349 | 4 | no gene near | 1455 |
| 348956 | 23.13 | 800901 | SCAFFOLD116028_5928 | 7 | ICA1 islet cell | 1456 |
| | | | | | autoantigen 1 | |
| 348835 | 4.126 | 328465 | SCAFFOLD86728_4181 | 7 | near SLC25A30 | 1457 |
| | | | | | solute carrier | |
| | | | | | family 25 member | |
| | | | | | A30 | |
| 354228 | Un.2727 | 71680 | SCAFFOLD75727_10291 | 5 | no gene near | 1458 |
| 347398 | 23.88 | 1235214 | SCAFFOLD210862_10205 | 6 | near SLC35B3 | 1459 |
| | | | | | solute carrier | |
| | | | | | family 35 member | |
| | | | | | B3 | |
| 353710 | Un.1242 | 162535 | SCAFFOLD90782_1366 | 5? | near predicted | 1460 |
| | | | | | gene like | |
| | | | | | LOC513860 | |
| | | | | | hypothetical | |
| | | | | | protein similar to | |
| | | | | | reverse | |
| | | | | | transcriptase-like | |
| 349108 | Un.5136 | 27731 | SCAFFOLD171014_5604 | 7 | no gene near | 1461 |

Snp_id is the ParAllele SNP identifier,
scaffold-v2 is the version 2 scaffold,
bp is the location of the SNP in the scaffold,
scaffold-v1/IBISS4 gives a direct reference to the SNP,
Hsa is the human chromosome associated with the SNP and
Gene is the closest gene to the SNP.

TABLE 33
Associations between DNA markers and meat tenderness sorted in decreasing order
of statistical significance.

| snp_id | scaffold-v2 | bp | bases | | mean0 | SE | mean1 | SE | mean2 |
|---|---|---|---|---|---|---|---|---|---|
| 344371 | Un.2692 | 15944 | C/T | 344371 | 0.56 | 0.17 | −0.06 | 0.08 | −0.05 |
| 348385 | Un.3082 | 36364 | A/G | 348385 | 0.26 | 0.08 | 0.10 | 0.13 | −0.16 |
| 348956 | 23.13 | 800901 | C/G | 348956 | −0.09 | 0.06 | 0.24 | 0.08 | −0.16 |
| 350387 | 25.18 | 276632 | A/G | 350387 | 0.21 | 0.12 | −0.25 | 0.09 | 0.10 |
| 354161 | Un.3082 | 36240 | C/T | 354161 | −0.14 | 0.06 | 0.25 | 0.08 | 0.07 |
| 347398 | 23.88 | 1235214 | G/T | 347398 | −0.12 | 0.06 | 0.21 | 0.07 | −0.04 |
| 348835 | 4.126 | 328465 | C/T | 348835 | 0.02 | 0.05 | 0.08 | 0.13 | −0.89 |
| 354228 | Un.2727 | 71680 | A/G | 354228 | −0.09 | 0.06 | 0.32 | 0.11 | −0.05 |
| 349108 | Un.5136 | 27731 | C/T | 349108 | 0.08 | 0.05 | −0.39 | 0.08 | −0.15 |
| 353710 | Un.1242 | 162535 | A/T | 353710 | 0.03 | 0.05 | 0.05 | 0.09 | −0.70 |

| snp_id | SE | N | Freq | a | k | a | tmax | PermP | SEQ ID NO: |
|---|---|---|---|---|---|---|---|---|---|
| 344371 | 0.05 | 168 | 0.25 | 0.31 | −1.04 | 0.46 | 4.04 | 0.0000 | 1452 |
| 348385 | 0.06 | 142 | 0.43 | 0.21 | 0.26 | 0.20 | 4.43 | 0.0000 | 1453 |
| 348956 | 0.09 | 168 | 0.77 | 0.03 | 10.93 | 0.23 | 3.41 | 0.0000 | 1456 |
| 350387 | 0.05 | 168 | 0.20 | 0.06 | −7.25 | 0.30 | 3.48 | 0.0000 | 1454 |
| 354161 | 0.11 | 168 | 0.73 | −0.11 | −2.70 | 0.03 | 4.06 | 0.0000 | 1455 |
| 347398 | 0.13 | 168 | 0.74 | −0.04 | −7.09 | 0.10 | 3.49 | 0.0001 | 1459 |
| 348835 | 0.37 | 164 | 0.89 | 0.45 | 1.14 | 0.85 | 3.72 | 0.0001 | 1457 |
| 354228 | 0.09 | 155 | 0.67 | −0.02 | −18.90 | 0.11 | 3.34 | 0.0001 | 1458 |
| 349108 | 0.30 | 168 | 0.91 | 0.11 | −3.07 | −0.17 | 3.72 | 0.0002 | 1461 |
| 353710 | 0.21 | 167 | 0.87 | 0.37 | 1.05 | 0.65 | 3.46 | | 1460 |

Locus is the ParAllele identifiers of the polymorphisms,
N is the number of genotypes, mean_0 is the mean meat tenderness for genotype 0,
mean_1 is the mean for genotype 1, and
mean_2 is the mean of genotype 2,
SE is the standard error,
a is the additive effect,
k is the dominance effect,
alpha is the average effect of allele substitutions,
tmax is the value of the t test, and
PermP is the P value determined from 100,000 permutation tests expressed as a positive integer.
A minus value for a means the mean of genotype 0 is lower than the mean of genotype 2 and a minus value for alpha means that selecting for allele 0 will reduce the average values of meat tenderness in the population.
Scaffold-v2 is the Draft version 2 Bovine genome sequence,
bp is the location in base pairs in the scaffold, and
bases are the alternative bases

TABLE 34
Associations between DNA markers and meat tenderness sorted in decreasing order
of statistical significance.

| snp_id | scaffold-v2 | bp | bases | | mean0 | SE | mean1 | SE | mean2 |
|---|---|---|---|---|---|---|---|---|---|
| 344295 | 19.45 | 215847 | C/T | 344295 | 0.18 | 0.07 | −0.17 | 0.06 | 0.03 |
| 347447 | 8.125 | 303271 | A/T | 347447 | 0.93 | 0.26 | 0.02 | 0.08 | −0.03 |
| 352858 | Un.3226 | 26597 | A/G | 352858 | −0.34 | 0.60 | 1.05 | 0.29 | −0.01 |
| 347467 | 13.11 | 127806 | C/T | 347467 | −0.23 | 0.07 | 0.25 | 0.08 | −0.08 |
| 349904 | Un.3560 | 28939 | A/T | 349904 | −0.03 | 0.07 | −1.06 | 0.52 | 0.07 |
| 344183 | 9.47 | 71878 | A/T | 344183 | −0.24 | 0.06 | −0.34 | 0.09 | 0.08 |
| 345548 | Un.1972 | 60288 | A/G | 345548 | −0.56 | 0.21 | 0.08 | 0.07 | 0.02 |
| 351609 | Un.7793 | 21627 | C/T | 351609 | 1.06 | 0.28 | 0.07 | 0.15 | −0.01 |
| 350871 | NULL | NULL | C/T | 350871 | 0.17 | 0.15 | 0.23 | 0.09 | −0.09 |
| 350305 | 15.71 | 229708 | C/T | 350305 | 0.06 | 0.10 | 0.16 | 0.07 | −0.19 |
| 343608 | Un.2073 | 135761 | A/G | 343608 | −0.15 | 0.10 | 0.24 | 0.09 | −0.06 |
| 350930 | Un.7793 | 21344 | C/T | 350930 | 1.06 | 0.28 | 0.07 | 0.15 | −0.03 |
| 347497 | Un.7793 | 21278 | G/T | 347497 | 1.06 | 0.28 | 0.07 | 0.15 | −0.03 |
| 342489 | Un.100 | 270891 | C/G | 342489 | −0.02 | 0.06 | 0.18 | 0.08 | −0.52 |
| 347190 | 25.67 | 602153 | A/G | 347190 | 0.01 | 0.08 | 0.15 | 0.06 | −0.18 |
| 349724 | Un.1242 | 162777 | C/T | 349724 | −0.70 | 0.21 | 0.03 | 0.08 | 0.04 |
| 348335 | 4.106 | 238981 | A/T | 348335 | −0.11 | 0.12 | 0.23 | 0.09 | −0.08 |
| 346457 | 1.69 | 764055 | C/T | 346457 | 0.10 | 0.05 | −0.21 | 0.08 | 0.06 |
| 346899 | 5.146 | 420627 | A/C | 346899 | −0.14 | 0.13 | 0.21 | 0.08 | −0.07 |
| 354051 | 15.71 | 230123 | A/G | 354051 | −0.19 | 0.07 | 0.16 | 0.07 | 0.04 |
| 344214 | 11.153 | 822417 | G/T | 344214 | 0.00 | 0.08 | 0.12 | 0.06 | −0.27 |
| 345920 | 1.265 | 40312 | A/G | 345920 | 0.02 | 0.07 | −0.13 | 0.06 | 0.24 |
| 350159 | Un.7793 | 21323 | A/G | 350159 | 1.06 | 0.28 | 0.07 | 0.15 | −0.03 |
| 352489 | 24.82 | 100372 | A/C | 352489 | −0.05 | 0.05 | 0.33 | 0.13 | 0.11 |
| 353900 | Un.351 | 230754 | A/T | 353900 | 0.06 | 0.05 | −0.30 | 0.12 | NaN |
| 354010 | Un.3725 | 48688 | A/T | 354010 | −0.53 | 0.20 | 0.12 | 0.10 | 0.02 |
| 348544 | 8.128 | 88667 | A/G | 348544 | 0.02 | 0.05 | 0.09 | 0.13 | −0.63 |
| 347844 | 3.171 | 110891 | C/T | 347844 | −0.09 | 0.05 | 0.21 | 0.09 | 0.28 |
| 348208 | 2.93 | 1121519 | A/C | 348208 | −0.28 | 0.14 | 0.18 | 0.07 | −0.07 |
| 344568 | 23.13 | 720587 | C/T | 344568 | 0.04 | 0.04 | −0.48 | 0.22 | −0.06 |
| 346260 | 23.13 | 720405 | C/T | 346260 | 0.04 | 0.04 | −0.48 | 0.22 | −0.06 |
| 346803 | 11.153 | 1473417 | C/T | 346803 | −0.17 | 0.07 | 0.14 | 0.07 | 0.11 |
| 345870 | Un.4101 | 41739 | A/T | 345870 | −0.22 | 0.09 | 0.08 | 0.08 | 0.11 |
| 349884 | 24.42 | 281842 | A/G | 349884 | −0.01 | 0.04 | 0.00 | 0.17 | 0.85 |
| 351830 | Un.240 | 189153 | A/G | 351830 | 0.08 | 0.06 | 0.05 | 0.07 | −0.31 |
| 352396 | Un.240 | 189053 | C/T | 352396 | −0.31 | 0.12 | 0.05 | 0.07 | 0.08 |
| 343102 | 20.77 | 1004580 | A/G | 343102 | −0.10 | 0.06 | 0.05 | 0.08 | 0.25 |
| 346207 | 21.26 | 732301 | C/T | 346207 | −0.40 | 0.16 | 0.07 | 0.07 | 0.02 |
| 346237 | 23.44 | 302124 | A/G | 346237 | −0.16 | 0.08 | 0.12 | 0.07 | 0.06 |
| 346881 | Un.240 | 189525 | C/T | 346881 | −0.31 | 0.12 | 0.05 | 0.07 | 0.08 |
| 349455 | Un.142 | 17692 | A/G | 349455 | −0.06 | 0.05 | 0.29 | 0.09 | −0.30 |
| 354162 | Un.3082 | 36175 | G/T | 354162 | 0.02 | 0.14 | 0.21 | 0.08 | −0.09 |
| 345666 | 12.13 | 232214 | A/G | 345666 | 0.20 | 0.07 | −0.05 | 0.08 | −0.08 |
| 347009 | 4.114 | 46740 | A/G | 347009 | 0.03 | 0.07 | 0.08 | 0.07 | −0.29 |
| 348378 | 12.13 | 231913 | C/T | 348378 | 0.20 | 0.07 | −0.05 | 0.08 | −0.08 |
| 342943 | NULL | NULL | C/T | 342943 | 0.08 | 0.06 | −0.01 | 0.08 | −0.33 |
| 347933 | 19.124 | 83735 | A/C | 347933 | NaN | NaN | −0.43 | 0.17 | 0.05 |
| 347783 | 11.173 | 210242 | A/T | 347783 | 0.33 | 0.15 | 0.05 | 0.08 | −0.07 |
| 351157 | 6.133 | 323039 | A/T | 351157 | 0.91 | 0.35 | 0.07 | 0.09 | −0.04 |
| 348153 | 24.42 | 282071 | C/T | 348153 | −0.01 | 0.04 | 0.04 | 0.18 | 0.85 |
| 345455 | 6.141 | 521629 | A/C | 345455 | −0.18 | 0.10 | −0.10 | 0.10 | 0.15 |
| 351473 | 11.163 | 280847 | A/G | 351473 | 0.25 | 0.12 | 0.20 | 0.11 | −0.11 |
| 349248 | 12.13 | 231848 | A/T | 349248 | −0.08 | 0.07 | −0.05 | 0.08 | 0.20 |
| 351790 | Un.7299 | 10550 | A/C | 351790 | 0.07 | 0.06 | −0.21 | 0.09 | 0.26 |
| 347189 | 24.42 | 284365 | A/T | 347189 | 0.85 | 0.39 | 0.07 | 0.15 | −0.02 |
| 343732 | 29.102 | 498923 | A/G | 343732 | 0.29 | 0.12 | 0.03 | 0.09 | −0.06 |
| 344074 | 12.13 | 231935 | C/T | 344074 | −0.08 | 0.07 | −0.04 | 0.09 | 0.18 |
| 343015 | 11.153 | 822202 | C/T | 343015 | −0.24 | 0.10 | 0.10 | 0.07 | 0.00 |
| 349117 | Un.12818 | 12771 | A/G | 349117 | −0.02 | 0.07 | −0.09 | 0.07 | 0.27 |
| 351241 | 8.66 | 160865 | C/T | 351241 | 0.06 | 0.24 | −0.19 | 0.09 | 0.08 |
| 352073 | 2.74 | 231913 | A/G | 352073 | 0.04 | 0.12 | −0.13 | 0.05 | 0.15 |
| 346121 | Un.3146 | 10954 | A/T | 346121 | 0.15 | 0.07 | −0.15 | 0.07 | −0.02 |
| 348916 | 23.44 | 301948 | G/T | 348916 | −0.19 | 0.08 | 0.11 | 0.07 | 0.04 |
| 351544 | Un.16047 | 5152 | C/T | 351544 | −0.01 | 0.05 | 0.16 | 0.09 | −0.37 |
| 352834 | 18.63 | 185411 | A/G | 352834 | −0.41 | 0.11 | −0.35 | 0.12 | 0.08 |
| 344400 | 1.57 | 71230 | C/T | 344400 | 0.10 | 0.06 | −0.17 | 0.07 | 0.17 |
| 343146 | Un.5180 | 45826 | A/T | 343146 | 0.07 | 0.05 | −0.24 | 0.10 | −0.06 |
| 344529 | 19.113 | 545504 | C/T | 344529 | −0.09 | 0.06 | 0.18 | 0.08 | −0.02 |
| 349950 | Un.6970 | 27673 | C/T | 349950 | 0.05 | 0.08 | 0.12 | 0.07 | −0.16 |
| 343712 | 9.111 | 1158162 | A/G | 343712 | 0.42 | 0.18 | 0.04 | 0.09 | −0.05 |
| 343738 | 14.116 | 14890 | C/T | 343738 | −0.07 | 0.05 | 0.20 | 0.08 | −0.16 |
| 343747 | 21.26 | 733216 | A/G | 343747 | −0.01 | 0.06 | 0.07 | 0.07 | −0.40 |
| 346307 | 20.77 | 286925 | C/T | 346307 | −0.24 | 0.09 | 0.09 | 0.07 | 0.05 |
| 347494 | Un.2612 | 14892 | C/T | 347494 | 0.07 | 0.06 | −0.21 | 0.08 | 0.20 |
| 350337 | 1.265 | 40249 | C/T | 350337 | 0.24 | 0.12 | −0.11 | 0.07 | 0.02 |
| 353281 | 10.98 | 96423 | A/G | 353281 | 0.06 | 0.16 | 0.19 | 0.08 | −0.08 |
| 351998 | 10.95 | 27011 | C/T | 351998 | 0.01 | 0.07 | 0.12 | 0.07 | −0.20 |
| 342821 | Un.1309 | 130852 | C/T | 342821 | −0.35 | 0.14 | 0.07 | 0.07 | 0.04 |
| 352336 | 20.46 | 162047 | A/T | 352336 | 0.13 | 0.06 | −0.14 | 0.06 | −0.15 |
| 354185 | Un.4101 | 41624 | C/T | 354185 | 0.08 | 0.07 | 0.10 | 0.07 | −0.21 |
| 347765 | 19.3 | 387941 | C/T | 347765 | 0.03 | 0.05 | 0.08 | 0.10 | −0.41 |
| 350772 | 29.22 | 353481 | C/T | 350772 | 0.10 | 0.05 | −0.11 | 0.09 | −0.44 |
| 349872 | Un.555 | 229185 | A/G | 349872 | 0.40 | 0.14 | −0.03 | 0.11 | −0.03 |
| 352390 | Un.351 | 230899 | A/G | 352390 | 0.06 | 0.05 | −0.28 | 0.12 | 0.27 |
| 350508 | 9.28 | 508356 | A/G | 350508 | −0.30 | 0.13 | 0.11 | 0.08 | 0.03 |
| 351413 | 24.42 | 282006 | C/T | 351413 | −0.01 | 0.04 | 0.04 | 0.19 | 0.85 |
| 346375 | 7.42 | 254403 | A/G | 346375 | −0.01 | 0.16 | 0.18 | 0.08 | −0.08 |
| 346361 | 19.42 | 1952799 | C/T | 346361 | 0.05 | 0.06 | −0.13 | 0.07 | 0.32 |
| 348493 | Un.1207 | 26097 | C/T | 348493 | 0.14 | 0.09 | −0.16 | 0.06 | 0.05 |
| 350213 | 5.175 | 641103 | G/T | 350213 | −0.05 | 0.05 | 0.31 | 0.13 | 0.21 |
| 349400 | Un.11944 | 14854 | C/G | 349400 | 0.13 | 0.12 | 0.20 | 0.08 | −0.09 |
| 350994 | 29.3 | 130795 | A/G | 350994 | 0.16 | 0.17 | −0.55 | 0.29 | 0.04 |
| 348268 | 5.17 | 52727 | A/C | 348268 | −0.92 | 0.62 | NaN | NaN | 0.00 |
| 352775 | Un.5392 | 43292 | C/T | 352775 | 0.04 | 0.09 | −0.15 | 0.08 | 0.13 |
| 344576 | 3.162 | 380554 | G/T | 344576 | −0.36 | 0.18 | −0.07 | 0.06 | 0.14 |
| 345852 | Un.888 | 42298 | A/G | 345852 | −0.01 | 0.05 | 0.18 | 0.09 | −0.29 |
| 348677 | 25.18 | 276801 | A/G | 348677 | 0.06 | 0.05 | −0.31 | 0.16 | 0.09 |
| 343726 | Un.64 | 830575 | A/G | 343726 | 0.72 | 0.37 | −0.05 | 0.11 | −0.01 |
| 348058 | 2.196 | 32036 | C/T | 348058 | 0.03 | 0.06 | 0.06 | 0.07 | −0.36 |
| 350890 | 27.58 | 550606 | C/T | 350890 | −0.10 | 0.05 | 0.17 | 0.09 | 0.14 |
| 352304 | Un.4101 | 41415 | A/T | 352304 | 0.10 | 0.07 | 0.07 | 0.08 | −0.20 |
| 351190 | 26.65 | 637074 | G/T | 351190 | 0.26 | 0.11 | −0.00 | 0.06 | −0.12 |
| 353955 | 22.21 | 72276 | A/G | 353955 | 0.06 | 0.05 | −0.28 | 0.11 | 0.36 |
| 344114 | 1.253 | 32791 | C/T | 344114 | −0.43 | 0.11 | −0.05 | 0.08 | 0.09 |
| 346646 | 22.46 | 1024610 | G/T | 346646 | −0.08 | 0.07 | 0.02 | 0.07 | 0.29 |
| 344573 | Un.80 | 791689 | C/G | 344573 | −0.05 | 0.05 | 0.24 | 0.10 | −0.09 |
| 344761 | 19.36 | 9351 | G/T | 344761 | 0.00 | 0.07 | 0.01 | 0.06 | −0.40 |
| 346257 | 16.119 | 23767 | C/T | 346257 | 0.03 | 0.05 | 0.12 | 0.16 | −0.53 |
| 350520 | 16.7 | 90701 | A/G | 350520 | −0.16 | 0.06 | 0.15 | 0.08 | 0.10 |
| 347204 | 26.68 | 145643 | A/G | 347204 | −0.22 | NaN | −0.34 | 0.15 | 0.04 |
| 348940 | 23.13 | 720641 | A/G | 348940 | 0.04 | 0.05 | −0.48 | 0.22 | −0.06 |
| 350154 | 11.154 | 294233 | A/G | 350154 | 0.27 | 0.11 | −0.10 | 0.07 | −0.01 |
| 342951 | Un.1681 | 173112 | A/G | 342951 | −0.02 | 0.05 | 0.44 | 0.19 | −0.03 |
| 344064 | 3.181 | 604919 | C/T | 344064 | 0.06 | 0.09 | −0.09 | 0.06 | 0.24 |
| 344260 | Un.7963 | 20271 | A/C | 344260 | −0.04 | 0.05 | −0.20 | 0.16 | 0.27 |
| 347268 | 11.173 | 33171 | C/G | 347268 | −0.22 | 0.13 | −0.02 | 0.06 | 0.14 |
| 350945 | Un.1947 | 57836 | A/G | 350945 | 0.00 | 0.06 | −0.06 | 0.07 | 0.53 |
| 353698 | 2.41 | 199334 | C/T | 353698 | −0.30 | 0.11 | 0.17 | 0.10 | 0.01 |
| 353972 | 1.194 | 283477 | C/T | 353972 | −0.05 | 0.05 | 0.17 | 0.24 | 0.19 |
| 350771 | 15.127 | 476703 | C/T | 350771 | −0.07 | 0.06 | 0.18 | 0.07 | −0.10 |
| 351373 | Un.120 | 332372 | A/G | 351373 | 0.08 | 0.07 | 0.01 | 0.07 | −0.32 |
| 348026 | Un.8890 | 18036 | A/G | 348026 | 0.09 | 0.06 | −0.19 | 0.10 | 0.21 |
| 353550 | Un.1173 | 227678 | A/G | 353550 | 0.06 | 0.05 | −0.12 | 0.13 | −0.78 |
| 348198 | 24.15 | 448252 | A/C | 348198 | −0.02 | 0.07 | 0.20 | 0.11 | −0.15 |
| 349814 | Un.120 | 332325 | C/T | 349814 | 0.08 | 0.07 | 0.01 | 0.07 | −0.32 |
| 346089 | 9.87 | 729686 | G/T | 346089 | 0.38 | 0.19 | −0.02 | 0.11 | −0.02 |
| 343284 | 11.192 | 299737 | C/T | 343284 | −0.13 | 0.07 | 0.14 | 0.07 | −0.03 |
| 344145 | 4.56 | 182359 | C/T | 344145 | 0.32 | 0.18 | 0.07 | 0.09 | −0.07 |
| 352054 | 24.42 | 281787 | A/G | 352054 | 0.85 | 0.39 | −0.07 | 0.21 | −0.00 |
| 349170 | 5.49 | 6863 | A/T | 349170 | 0.25 | 0.09 | −0.02 | 0.07 | −0.08 |
| 349944 | Un.11 | 1300793 | A/G | 349944 | 0.12 | 0.06 | −0.33 | 0.18 | 0.05 |
| 351679 | 2.196 | 20030 | A/C | 351679 | −0.27 | 0.13 | 0.03 | 0.06 | 0.09 |
| 343328 | 25.31 | 316043 | A/G | 343328 | −0.05 | 0.06 | 0.20 | 0.09 | −0.08 |
| 343990 | Un.760 | 280889 | C/T | 343990 | 0.03 | 0.09 | 0.14 | 0.08 | −0.14 |
| 354309 | Un.12 | 725041 | C/T | 354309 | 0.25 | 0.09 | −0.03 | 0.06 | −0.10 |
| 344338 | 10.21 | 154297 | C/T | 344338 | 0.00 | 0.05 | −0.03 | 0.08 | 0.86 |
| 349116 | Un.12818 | 12550 | C/T | 349116 | −0.02 | 0.07 | −0.09 | 0.07 | 0.27 |
| 345082 | 15.4 | 379496 | A/G | 345082 | −0.02 | 0.04 | 0.27 | 0.13 | −0.15 |
| 345971 | 30.75 | 281410 | C/T | 345971 | −0.01 | 0.04 | 0.77 | 0.41 | NaN |
| 350209 | 20.71 | 229003 | G/T | 350209 | −0.07 | 0.06 | 0.20 | 0.09 | −0.05 |
| 344111 | NULL | NULL | A/C | 344111 | −0.02 | 0.07 | 0.15 | 0.07 | −0.16 |
| 346429 | Un.12818 | 12728 | C/T | 346429 | −0.02 | 0.07 | −0.08 | 0.07 | 0.27 |
| 351102 | 5.98 | 61033 | A/G | 351102 | 0.17 | 0.08 | −0.01 | 0.09 | −0.12 |
| 345331 | 25.54 | 9023 | A/G | 345331 | −0.14 | 0.07 | 0.11 | 0.06 | 0.10 |
| 347729 | 16.53 | 53443 | A/G | 347729 | −0.11 | 0.12 | 0.17 | 0.07 | −0.08 |
| 351561 | 6.78 | 359331 | A/T | 351561 | −0.30 | 0.10 | 0.06 | 0.08 | 0.06 |
| 351747 | Un.78 | 386183 | A/G | 351747 | −0.19 | 0.10 | 0.14 | 0.07 | −0.01 |
| 353461 | Un.1786 | 134734 | G/T | 353461 | 0.04 | 0.05 | −0.32 | 0.11 | 0.46 |
| 353888 | Un.688 | 9871 | C/G | 353888 | 0.03 | 0.07 | 0.09 | 0.07 | −0.28 |
| 345708 | 20.5 | 109635 | A/G | 345708 | 0.56 | 0.33 | 0.07 | 0.09 | −0.05 |
| 353431 | Un.113 | 46312 | C/T | 353431 | −0.52 | 0.16 | 0.09 | 0.13 | 0.02 |
| 347137 | 16.7 | 503497 | G/T | 347137 | −0.15 | 0.11 | 0.00 | 0.07 | 0.18 |
| 351781 | 16.53 | 53223 | A/G | 351781 | −0.11 | 0.12 | 0.17 | 0.07 | −0.08 |
| 345063 | 20.17 | 280653 | G/T | 345063 | −0.06 | 0.06 | 0.20 | 0.08 | −0.27 |
| 351730 | Un.5686 | 11094 | C/T | 351730 | −0.26 | 0.08 | 0.04 | 0.09 | 0.06 |
| 354456 | 8.1 | 36180 | C/T | 354456 | 0.09 | 0.06 | −0.00 | 0.09 | −0.29 |
| 343396 | 3.161 | 8871 | C/T | 343396 | 0.00 | 0.06 | −0.12 | 0.07 | 0.24 |
| 347117 | NULL | NULL | A/G | 347117 | 0.18 | 0.07 | −0.10 | 0.07 | −0.06 |
| 348666 | 14.99 | 87546 | A/G | 348666 | 0.00 | 0.08 | 0.09 | 0.06 | −0.23 |
| 352499 | 13.25 | 535313 | A/G | 352499 | 0.17 | 0.07 | −0.09 | 0.07 | 0.00 |
| 344347 | 26.65 | 636814 | C/T | 344347 | 0.82 | 0.41 | −0.09 | 0.07 | 0.03 |
| 349557 | NULL | NULL | C/G | 349557 | 0.04 | 0.06 | −0.12 | 0.07 | 0.35 |
| 351300 | NULL | NULL | C/T | 351300 | −0.09 | 0.06 | 0.17 | 0.07 | 0.12 |
| 352151 | 9.61 | 14338 | A/T | 352151 | 0.06 | 0.05 | −0.10 | 0.11 | −0.56 |
| 345384 | 8.1 | 327168 | G/T | 345384 | 0.18 | 0.08 | −0.12 | 0.08 | −0.04 |
| 347400 | 3.138 | 666684 | C/G | 347400 | −0.02 | 0.07 | 0.15 | 0.07 | −0.15 |
| 347055 | Un.10793 | 15469 | A/G | 347055 | −0.12 | 0.12 | −0.10 | 0.06 | 0.16 |
| 353259 | 20.71 | 83444 | C/T | 353259 | −0.48 | 0.14 | 0.17 | 0.13 | −0.01 |
| 349787 | 28.51 | 78070 | C/T | 349787 | −0.04 | 0.06 | 0.16 | 0.07 | −0.20 |

| snp_id | SE | N | Freq | a | k | a | tmax | PermP | SEQ ID NO: |
|---|---|---|---|---|---|---|---|---|---|
| 344295 | 0.10 | 168 | 0.62 | 0.07 | −3.84 | 0.00 | 3.67 | 0.0003 | 1462 |
| 347447 | 0.05 | 168 | 0.13 | 0.48 | −0.90 | 0.80 | 4.03 | 0.0003 | 1463 |
| 352858 | 0.04 | 166 | 0.04 | −0.16 | −7.42 | −1.29 | 4.33 | 0.0004 | 1464 |
| 347467 | 0.05 | 168 | 0.16 | −0.07 | −5.68 | −0.35 | 3.39 | 0.0005 | 1465 |
| 349904 | 0.06 | 164 | 0.44 | −0.05 | 22.03 | 0.09 | 3.32 | 0.0005 | 1466 |
| 344183 | 0.05 | 168 | 0.10 | −0.16 | 1.59 | 0.05 | 3.43 | 0.0006 | 1467 |
| 345548 | 0.05 | 167 | 0.22 | −0.29 | −1.21 | −0.49 | 3.44 | 0.0006 | 1468 |
| 351609 | 0.05 | 164 | 0.09 | 0.54 | −0.84 | 0.90 | 3.40 | 0.0006 | 1469 |
| 350871 | 0.05 | 168 | 0.20 | 0.13 | 1.52 | 0.01 | 3.22 | 0.0008 | 1470 |
| 350305 | 0.07 | 168 | 0.37 | 0.12 | 1.77 | 0.07 | 3.57 | 0.0009 | 1471 |
| 343608 | 0.07 | 155 | 0.30 | −0.05 | −7.42 | −0.18 | 2.95 | 0.0011 | 1472 |
| 350930 | 0.05 | 168 | 0.09 | 0.54 | −0.82 | 0.91 | 3.43 | 0.0012 | 1473 |
| 347497 | 0.05 | 168 | 0.09 | 0.54 | −0.82 | 0.91 | 3.43 | 0.0013 | 1474 |
| 342489 | 0.15 | 165 | 0.80 | 0.25 | 1.81 | 0.52 | 3.25 | 0.0014 | 1475 |
| 347190 | 0.09 | 168 | 0.49 | 0.09 | 2.53 | 0.09 | 3.05 | 0.0014 | 1476 |
| 349724 | 0.05 | 168 | 0.14 | −0.37 | −0.98 | −0.63 | 3.44 | 0.0014 | 1477 |
| 348335 | 0.05 | 166 | 0.33 | −0.02 | −20.93 | −0.13 | 3.14 | 0.0015 | 1478 |
| 346457 | 0.33 | 167 | 0.83 | 0.02 | −15.43 | −0.17 | 3.18 | 0.0016 | 1479 |
| 346899 | 0.05 | 168 | 0.18 | −0.04 | −8.80 | −0.24 | 2.82 | 0.0016 | 1480 |
| 354051 | 0.11 | 167 | 0.63 | −0.11 | −2.04 | −0.05 | 3.57 | 0.0016 | 1481 |
| 344214 | 0.11 | 160 | 0.60 | 0.14 | 1.90 | 0.19 | 3.25 | 0.0018 | 1482 |
| 345920 | 0.12 | 167 | 0.57 | −0.11 | 2.37 | −0.14 | 2.96 | 0.0018 | 1483 |
| 350159 | 0.05 | 165 | 0.09 | 0.55 | −0.81 | 0.91 | 3.46 | 0.0018 | 1484 |
| 352489 | 0.03 | 168 | 0.92 | −0.08 | −3.93 | 0.18 | 2.99 | 0.0018 | 1485 |
| 353900 | NaN | 165 | 0.91 | NaN | NaN | NaN | 3.08 | 0.0018 | 1486 |
| 354010 | 0.05 | 168 | 0.19 | −0.27 | −1.37 | −0.51 | 3.29 | 0.0018 | 1487 |
| 348544 | 0.28 | 168 | 0.87 | 0.33 | 1.20 | 0.62 | 3.09 | 0.0019 | 1488 |
| 347844 | 0.15 | 167 | 0.81 | −0.19 | −0.64 | −0.11 | 2.88 | 0.0022 | 1489 |
| 348208 | 0.06 | 159 | 0.25 | −0.11 | −3.38 | −0.28 | 2.99 | 0.0022 | 1490 |
| 344568 | NaN | 168 | 0.96 | 0.05 | −9.45 | −0.38 | 2.90 | 0.0024 | 1491 |
| 346260 | NaN | 168 | 0.96 | 0.05 | −9.45 | −0.38 | 2.90 | 0.0024 | 1492 |
| 346803 | 0.08 | 168 | 0.60 | −0.14 | −1.16 | −0.11 | 2.94 | 0.0025 | 1493 |
| 345870 | 0.07 | 165 | 0.45 | −0.16 | −0.86 | −0.18 | 2.95 | 0.0029 | 1494 |
| 349884 | 0.39 | 168 | 0.91 | −0.43 | 0.97 | −0.77 | 2.77 | 0.0029 | 1495 |
| 351830 | 0.12 | 168 | 0.65 | 0.20 | 0.86 | 0.25 | 3.07 | 0.0029 | 1496 |
| 352396 | 0.06 | 168 | 0.35 | −0.20 | −0.87 | −0.25 | 3.08 | 0.0029 | 1497 |
| 343102 | 0.12 | 162 | 0.62 | −0.17 | 0.17 | −0.18 | 2.94 | 0.0030 | 1498 |
| 346207 | 0.06 | 167 | 0.16 | −0.21 | −1.23 | −0.39 | 2.89 | 0.0030 | 1499 |
| 346237 | 0.10 | 167 | 0.56 | −0.11 | −1.49 | −0.09 | 2.79 | 0.0030 | 1500 |
| 346881 | 0.06 | 168 | 0.35 | −0.20 | −0.87 | −0.25 | 3.08 | 0.0030 | 1501 |
| 349455 | 0.18 | 166 | 0.84 | 0.12 | 3.97 | 0.44 | 3.29 | 0.0030 | 1502 |
| 354162 | 0.06 | 167 | 0.19 | 0.05 | 4.72 | −0.10 | 3.13 | 0.0030 | 1503 |
| 345666 | 0.07 | 168 | 0.47 | 0.14 | −0.75 | 0.15 | 2.82 | 0.0031 | 1504 |
| 347009 | 0.10 | 165 | 0.64 | 0.16 | 1.33 | 0.22 | 2.94 | 0.0031 | 1505 |
| 348378 | 0.07 | 168 | 0.47 | 0.14 | −0.75 | 0.15 | 2.82 | 0.0031 | 1506 |
| 342943 | 0.15 | 165 | 0.73 | 0.20 | 0.55 | 0.26 | 2.74 | 0.0032 | 1507 |
| 347933 | 0.05 | 159 | 0.04 | NaN | NaN | NaN | 2.94 | 0.0032 | 1508 |
| 347783 | 0.06 | 165 | 0.25 | 0.20 | −0.43 | 0.24 | 2.76 | 0.0033 | 1509 |
| 351157 | 0.05 | 166 | 0.14 | 0.48 | −0.77 | 0.74 | 2.84 | 0.0033 | 1510 |
| 348153 | 0.39 | 167 | 0.92 | −0.43 | 0.88 | −0.74 | 2.78 | 0.0034 | 1511 |
| 345455 | 0.07 | 144 | 0.31 | −0.17 | 0.51 | −0.13 | 2.53 | 0.0035 | 1512 |
| 351473 | 0.05 | 161 | 0.24 | 0.18 | 0.70 | 0.11 | 2.98 | 0.0036 | 1513 |
| 349248 | 0.07 | 167 | 0.53 | −0.14 | 0.80 | −0.15 | 2.82 | 0.0037 | 1514 |
| 351790 | 0.13 | 161 | 0.76 | −0.10 | 3.87 | −0.29 | 2.76 | 0.0037 | 1515 |
| 347189 | 0.04 | 168 | 0.10 | 0.44 | −0.78 | 0.71 | 2.86 | 0.0038 | 1516 |
| 343732 | 0.06 | 162 | 0.30 | 0.18 | −0.47 | 0.21 | 2.81 | 0.0040 | 1517 |
| 344074 | 0.07 | 168 | 0.52 | −0.13 | 0.71 | −0.14 | 2.69 | 0.0042 | 1518 |
| 343015 | 0.08 | 155 | 0.43 | −0.12 | −1.83 | −0.15 | 2.82 | 0.0043 | 1519 |
| 349117 | 0.12 | 167 | 0.66 | −0.14 | 1.56 | −0.21 | 2.79 | 0.0043 | 1520 |
| 351241 | 0.05 | 168 | 0.17 | −0.01 | 22.42 | 0.16 | 2.65 | 0.0043 | 1521 |
| 352073 | 0.08 | 161 | 0.36 | −0.06 | 4.09 | 0.01 | 2.84 | 0.0043 | 1522 |
| 346121 | 0.09 | 155 | 0.62 | 0.08 | −2.56 | 0.03 | 2.85 | 0.0044 | 1523 |
| 348916 | 0.10 | 158 | 0.53 | −0.11 | −1.59 | −0.10 | 2.80 | 0.0044 | 1524 |
| 351544 | 0.11 | 168 | 0.84 | 0.18 | 1.94 | 0.42 | 2.90 | 0.0044 | 1525 |
| 352834 | 0.05 | 165 | 0.06 | −0.24 | 0.75 | −0.08 | 3.02 | 0.0045 | 1526 |
| 344400 | 0.17 | 156 | 0.78 | −0.04 | 8.41 | −0.20 | 2.65 | 0.0046 | 1527 |
| 343146 | NaN | 161 | 0.90 | 0.06 | −3.85 | −0.13 | 2.67 | 0.0047 | 1528 |
| 344529 | 0.14 | 142 | 0.67 | −0.03 | −7.09 | 0.05 | 2.60 | 0.0047 | 1529 |
| 349950 | 0.08 | 168 | 0.46 | 0.11 | 1.58 | 0.10 | 2.63 | 0.0047 | 1530 |
| 343712 | 0.05 | 165 | 0.21 | 0.24 | −0.59 | 0.32 | 2.76 | 0.0048 | 1531 |
| 343738 | 0.16 | 168 | 0.80 | 0.04 | 7.70 | 0.23 | 2.82 | 0.0049 | 1532 |
| 343747 | 0.16 | 163 | 0.83 | 0.20 | 1.38 | 0.38 | 2.89 | 0.0049 | 1533 |
| 346307 | 0.07 | 167 | 0.39 | −0.14 | −1.30 | −0.18 | 2.70 | 0.0049 | 1534 |
| 347494 | 0.15 | 163 | 0.77 | −0.06 | 5.33 | −0.25 | 2.73 | 0.0049 | 1535 |
| 350337 | 0.07 | 168 | 0.43 | 0.11 | −2.21 | 0.14 | 2.78 | 0.0049 | 1536 |
| 353281 | 0.06 | 167 | 0.19 | 0.07 | 2.89 | −0.05 | 2.65 | 0.0049 | 1537 |
| 351998 | 0.09 | 167 | 0.59 | 0.11 | 2.06 | 0.14 | 2.74 | 0.0050 | 1538 |
| 342821 | 0.06 | 168 | 0.31 | −0.19 | −1.14 | −0.28 | 2.78 | 0.0051 | 1539 |
| 352336 | 0.11 | 156 | 0.69 | 0.14 | −0.95 | 0.09 | 2.87 | 0.0051 | 1540 |
| 354185 | 0.08 | 168 | 0.53 | 0.14 | 1.18 | 0.15 | 2.72 | 0.0051 | 1541 |
| 347765 | 0.18 | 168 | 0.82 | 0.22 | 1.23 | 0.40 | 2.71 | 0.0052 | 1542 |
| 350772 | 0.20 | 164 | 0.81 | 0.27 | 0.22 | 0.31 | 3.01 | 0.0052 | 1543 |
| 349872 | 0.05 | 167 | 0.21 | 0.21 | −1.02 | 0.34 | 2.86 | 0.0053 | 1544 |
| 352390 | 0.14 | 168 | 0.90 | −0.10 | 4.23 | −0.46 | 2.87 | 0.0053 | 1545 |
| 350508 | 0.06 | 167 | 0.34 | −0.16 | −1.51 | −0.24 | 2.78 | 0.0055 | 1546 |
| 351413 | 0.39 | 166 | 0.92 | −0.43 | 0.89 | −0.75 | 2.78 | 0.0055 | 1547 |
| 346375 | 0.06 | 165 | 0.25 | 0.04 | 5.85 | −0.07 | 2.63 | 0.0057 | 1548 |
| 346361 | 0.15 | 168 | 0.73 | −0.14 | 2.25 | −0.28 | 2.81 | 0.0058 | 1549 |
| 348493 | 0.08 | 168 | 0.51 | 0.04 | −6.18 | 0.04 | 2.78 | 0.0058 | 1550 |
| 350213 | 0.24 | 168 | 0.89 | −0.13 | −1.74 | 0.05 | 2.72 | 0.0058 | 1551 |
| 349400 | 0.06 | 168 | 0.23 | 0.11 | 1.59 | 0.02 | 2.69 | 0.0059 | 1552 |
| 350994 | 0.05 | 148 | 0.14 | 0.06 | −10.17 | 0.54 | 2.84 | 0.0059 | 1553 |
| 348268 | 0.05 | 148 | 0.02 | −0.46 | NaN | NaN | 2.77 | 0.0060 | 1554 |
| 352775 | 0.07 | 154 | 0.46 | −0.05 | 4.88 | −0.03 | 2.69 | 0.0061 | 1555 |
| 344576 | 0.06 | 168 | 0.29 | −0.25 | −0.18 | −0.27 | 2.95 | 0.0062 | 1556 |
| 345852 | 0.16 | 168 | 0.74 | 0.14 | 2.29 | 0.30 | 2.82 | 0.0062 | 1557 |
| 348677 | 0.20 | 167 | 0.90 | −0.02 | 20.10 | −0.33 | 2.69 | 0.0063 | 1558 |
| 343726 | 0.05 | 168 | 0.10 | 0.36 | −1.13 | 0.69 | 2.75 | 0.0065 | 1559 |
| 348058 | 0.15 | 168 | 0.76 | 0.20 | 1.13 | 0.32 | 2.77 | 0.0065 | 1560 |
| 350890 | 0.20 | 167 | 0.77 | −0.12 | −1.25 | −0.04 | 2.76 | 0.0065 | 1561 |
| 352304 | 0.09 | 166 | 0.55 | 0.15 | 0.77 | 0.17 | 2.79 | 0.0065 | 1562 |
| 351190 | 0.08 | 167 | 0.41 | 0.19 | −0.38 | 0.20 | 2.73 | 0.0066 | 1563 |
| 353955 | 0.29 | 157 | 0.87 | −0.15 | 3.23 | −0.51 | 2.85 | 0.0066 | 1564 |
| 344114 | 0.06 | 168 | 0.24 | −0.26 | −0.46 | −0.32 | 2.87 | 0.0068 | 1565 |
| 346646 | 0.11 | 165 | 0.65 | −0.19 | 0.45 | −0.21 | 2.56 | 0.0068 | 1566 |
| 344573 | 0.11 | 165 | 0.85 | 0.02 | 14.69 | 0.24 | 2.63 | 0.0069 | 1567 |
| 344761 | 0.16 | 152 | 0.67 | 0.20 | 1.02 | 0.27 | 2.75 | 0.0069 | 1568 |
| 346257 | 0.25 | 164 | 0.91 | 0.28 | 1.33 | 0.58 | 2.69 | 0.0069 | 1569 |
| 350520 | 0.09 | 164 | 0.55 | −0.13 | −1.41 | −0.11 | 3.07 | 0.0069 | 1570 |
| 347204 | 0.05 | 168 | 0.05 | −0.13 | 1.99 | 0.10 | 2.49 | 0.0070 | 1571 |
| 348940 | NaN | 167 | 0.96 | 0.05 | −9.73 | −0.38 | 2.88 | 0.0070 | 1572 |
| 350154 | 0.06 | 166 | 0.39 | 0.14 | −1.63 | 0.19 | 2.79 | 0.0070 | 1573 |
| 342951 | 0.18 | 159 | 0.86 | 0.01 | 85.17 | 0.34 | 2.73 | 0.0071 | 1574 |
| 344064 | 0.11 | 166 | 0.60 | −0.09 | 2.61 | −0.14 | 2.73 | 0.0071 | 1575 |
| 344260 | 0.13 | 167 | 0.80 | −0.16 | 1.98 | −0.34 | 2.66 | 0.0071 | 1576 |
| 347268 | 0.07 | 167 | 0.38 | −0.18 | −0.09 | −0.19 | 2.68 | 0.0072 | 1577 |
| 350945 | 0.18 | 168 | 0.81 | −0.26 | 1.23 | −0.46 | 2.99 | 0.0072 | 1578 |
| 353698 | 0.05 | 165 | 0.22 | −0.15 | −2.01 | −0.33 | 2.94 | 0.0074 | 1579 |
| 353972 | 0.07 | 146 | 0.60 | −0.12 | −0.81 | −0.10 | 2.71 | 0.0074 | 1580 |
| 350771 | 0.21 | 167 | 0.79 | 0.02 | 15.22 | 0.17 | 2.61 | 0.0076 | 1581 |
| 351373 | 0.12 | 168 | 0.67 | 0.20 | 0.65 | 0.25 | 2.82 | 0.0076 | 1582 |
| 348026 | 0.16 | 132 | 0.80 | −0.06 | 5.91 | −0.26 | 2.53 | 0.0077 | 1583 |
| 353550 | 0.20 | 167 | 0.90 | 0.42 | 0.57 | 0.61 | 2.63 | 0.0077 | 1584 |
| 348198 | 0.07 | 149 | 0.52 | 0.07 | 4.36 | 0.08 | 2.84 | 0.0078 | 1585 |
| 349814 | 0.12 | 168 | 0.67 | 0.20 | 0.65 | 0.25 | 2.82 | 0.0078 | 1586 |
| 346089 | 0.05 | 168 | 0.20 | 0.20 | −1.00 | 0.33 | 2.64 | 0.0079 | 1587 |
| 343284 | 0.10 | 167 | 0.57 | −0.05 | −4.26 | −0.02 | 2.71 | 0.0080 | 1588 |
| 344145 | 0.05 | 168 | 0.23 | 0.19 | −0.30 | 0.23 | 2.63 | 0.0080 | 1589 |
| 352054 | 0.04 | 168 | 0.07 | 0.42 | −1.16 | 0.85 | 2.66 | 0.0080 | 1590 |
| 349170 | 0.07 | 167 | 0.40 | 0.16 | −0.68 | 0.18 | 2.71 | 0.0081 | 1591 |
| 349944 | 0.10 | 136 | 0.70 | 0.04 | −11.24 | −0.13 | 2.74 | 0.0081 | 1592 |
| 351679 | 0.07 | 166 | 0.36 | −0.18 | −0.67 | −0.21 | 2.70 | 0.0081 | 1593 |
| 343328 | 0.14 | 160 | 0.71 | 0.02 | 16.38 | 0.12 | 2.49 | 0.0083 | 1594 |
| 343990 | 0.07 | 166 | 0.46 | 0.08 | 2.46 | 0.07 | 2.76 | 0.0083 | 1595 |
| 354309 | 0.08 | 167 | 0.44 | 0.18 | −0.60 | 0.19 | 2.82 | 0.0083 | 1596 |
| 344338 | 0.55 | 167 | 0.87 | −0.43 | 1.08 | −0.77 | 2.78 | 0.0084 | 1597 |
| 349116 | 0.12 | 165 | 0.65 | −0.14 | 1.54 | −0.21 | 2.83 | 0.0085 | 1598 |
| 345082 | 0.20 | 168 | 0.81 | 0.07 | 5.41 | 0.29 | 2.59 | 0.0086 | 1599 |
| 345971 | NaN | 167 | 0.99 | NaN | NaN | NaN | 2.70 | 0.0086 | 1600 |
| 350209 | 0.14 | 167 | 0.76 | −0.01 | −43.28 | 0.13 | 2.64 | 0.0086 | 1601 |
| 344111 | 0.09 | 165 | 0.50 | 0.07 | 3.51 | 0.07 | 2.69 | 0.0088 | 1602 |
| 346429 | 0.12 | 168 | 0.65 | −0.14 | 1.46 | −0.20 | 2.67 | 0.0088 | 1603 |
| 351102 | 0.08 | 162 | 0.51 | 0.14 | −0.22 | 0.14 | 2.64 | 0.0088 | 1604 |
| 345331 | 0.15 | 165 | 0.66 | −0.12 | −1.05 | −0.08 | 2.61 | 0.0089 | 1605 |
| 347729 | 0.06 | 168 | 0.37 | −0.02 | −16.57 | −0.08 | 2.60 | 0.0090 | 1606 |
| 351561 | 0.06 | 151 | 0.26 | −0.18 | −0.99 | −0.27 | 2.57 | 0.0091 | 1607 |
| 351747 | 0.07 | 168 | 0.41 | −0.09 | −2.70 | −0.13 | 2.73 | 0.0091 | 1608 |
| 353461 | 0.23 | 160 | 0.93 | −0.21 | 2.71 | −0.70 | 2.72 | 0.0091 | 1609 |
| 353888 | 0.13 | 168 | 0.65 | 0.15 | 1.37 | 0.22 | 2.71 | 0.0091 | 1610 |
| 345708 | 0.05 | 167 | 0.18 | 0.31 | −0.58 | 0.42 | 2.75 | 0.0092 | 1611 |
| 353431 | 0.05 | 167 | 0.13 | −0.27 | −1.25 | −0.53 | 2.70 | 0.0092 | 1612 |
| 347137 | 0.07 | 160 | 0.48 | −0.16 | 0.06 | −0.16 | 2.48 | 0.0093 | 1613 |
| 351781 | 0.06 | 168 | 0.37 | −0.02 | −16.57 | −0.08 | 2.60 | 0.0093 | 1614 |
| 345063 | 0.21 | 168 | 0.83 | 0.10 | 3.51 | 0.35 | 2.61 | 0.0094 | 1615 |
| 351730 | 0.07 | 161 | 0.34 | −0.16 | −0.90 | −0.21 | 2.68 | 0.0094 | 1616 |
| 354456 | 0.16 | 158 | 0.72 | 0.19 | 0.50 | 0.23 | 2.63 | 0.0094 | 1617 |
| 343396 | 0.14 | 167 | 0.67 | −0.12 | 1.97 | −0.20 | 2.62 | 0.0095 | 1618 |
| 347117 | 0.09 | 167 | 0.56 | 0.12 | −1.27 | 0.10 | 2.62 | 0.0095 | 1619 |
| 348666 | 0.10 | 168 | 0.62 | 0.12 | 1.74 | 0.17 | 2.68 | 0.0095 | 1620 |
| 352499 | 0.09 | 166 | 0.55 | 0.08 | −2.15 | 0.07 | 2.49 | 0.0095 | 1621 |
| 344347 | 0.06 | 167 | 0.17 | 0.39 | −1.32 | 0.74 | 2.85 | 0.0096 | 1622 |
| 349557 | 0.16 | 167 | 0.79 | −0.16 | 1.96 | −0.34 | 2.78 | 0.0096 | 1623 |
| 351300 | 0.13 | 167 | 0.74 | −0.10 | −1.52 | −0.03 | 2.59 | 0.0096 | 1624 |
| 352151 | 0.37 | 168 | 0.87 | 0.31 | 0.49 | 0.42 | 2.68 | 0.0096 | 1625 |
| 345384 | 0.07 | 157 | 0.46 | 0.11 | −1.73 | 0.13 | 2.68 | 0.0097 | 1626 |
| 347400 | 0.10 | 167 | 0.55 | 0.06 | 3.70 | 0.09 | 2.50 | 0.0097 | 1627 |
| 347055 | 0.07 | 166 | 0.36 | −0.14 | 0.82 | −0.11 | 2.70 | 0.0098 | 1628 |
| 353259 | 0.05 | 157 | 0.03 | −0.24 | −1.77 | −0.63 | 3.43 | 0.0098 | 1629 |
| 349787 | 0.12 | 168 | 0.74 | 0.08 | 3.50 | 0.22 | 2.68 | 0.0100 | 1630 |

Locus is the ParAllele identifiers of the polymorphisms,
N is the number of genotypes,
mean_0 is the mean meat tenderness for genotype 0,
mean_1 is the mean for genotype 1, and
mean_2 is the mean of genotype 2,
SE is the standard error,
a is the additive effect,
k is the dominance effect,
alpha is the average effect of allele substitutions,
tmax is the value of the t test, and
PermP is the P value determined from 100,000 permutation tests expressed as a positive integer.
A minus value for a means the mean of genotype 0 is lower than the mean of genotype 2, and a minus value for alpha means that selecting for allele 0 will reduce the average values of meat tenderness in the population.
Scaffold-v2 is the Draft version 2 Bovine genome sequence,
bp is the location in base pairs in the scaffold, and bases are the alternative bases.

A further SNP (SEQ ID NO:1631) with indent 343614 located at SCAFFOLD 344371_{—}609 has been found to be associated with LDPF.

### EXAMPLE 10

### Evaluation of a Cluster of SNP Around ParAllele SNP 343617 for Net Feed Intake (NFI)

It has been established that the SNP 343617 (SEQ ID NO:1632) lies in the gene encoding Synaptotagmin X (SYT 10). Synaptotagmins are integral membrane proteins of synaptic vesicle thought to serve as Ca(2+) sensors. They are involved in vesicular trafficking, and in the release of neurotransmitter at the synapse. While not wishing to be bound by theory, it is believed that metabolic rate is influenced by intracellular processes such as proton transport in the mitochondrion, while Sodium and Potassium pumps through the cell membrane are the major influences on basal metabolic rate. The trafficking associated with neurotransmitter release is thought to be part of the background energetics since there is a constant release of neurotransmitter and reconstitution of the neurotransmitter gradient, all requiring energy. Note that in SEQ ID NO: 1632 and the polymorphism is at position 3401 of scaffold 35407 and is a T/C polymorphism—the TT homozygote worse for NFI.

TABLE 35
Associations between DNA markers and Net Feed Intake sort in
decreasing order of statistical significance.

| run | locus | inf | N | mean_0 | SE | N | mean_1 | SE | N | mean_2 | SE | maxd | t | P | log(1/P) |
|---|---|---|---|---|---|---|---|---|---|---|---|---|---|---|---|
| 5278 | 343617 | Y | 84 | −0.09 | 1.75 | 48 | −0.22 | 1.78 | 18 | 1.95 | 1.40 | 2.174 | 8.193 | 0.000E+00 | 5.000 |

Locus and ident are the ParAllele identifiers of the polymorphisms,
inf is whether there are enough genotypes for each genotype for a proper test,
N is the number of genotypes,
mean_0 is the mean NFI for genotype 0,
mean_1 is the mean for genotype 1, and
mean_2 is the mean of genotype 2,
SD is the standard deviation,
maxd is the maximum difference between any of the three genotypes,
t is the value of the t test,
P is the P value determined from 100,000 permutation tests and
log(1/P) is the P value expressed as a positive integer.

TABLE 36
A further SNP with a high probability of being associated with NFI. The
ident is as in Table 35 and is the ParAllele identifiers. These are located on
the BGSP scaffolds and contigs which indicate the SNP location.

| | | BCM contig | |
|---|---|---|---|
| Locus | scaffold | Chrom. | Gene |
| 343617 | scaffold35407_3401 | contig282438 | Hsa12/Bta5SYT10 |
| | | | Synaptotagmin X incl |
| | | | exon4 |

This example describes the analysis of 3 additional SNP in the SYT10 gene genotyped on all the cattle of Example 1 with NFI measurements. These SNP flank the ParAllele SNP 343617 and form part of 16 potential SNP available in this part of the SYT10 gene. It was found that one of the SNP, SYTX3486, has an overall P<0.001 for the full sample, and it and SNP SYTX6146 have a more consistent association between NFI and genotype.

We chose other SNP near 343617 for further analysis, to determine if other SNP in the gene show more consistent associations to NFI. We obtained the SNP by examining raw sequence traces from the bovine genome sequence project in the vicinity of SYT10. We genotyped three SNP using standard methods for Taqman assays (ABI, Foster City, Calif.) and analysed the genotypes as in CRA report 3. The linkage disequilibrium estimates for the 4 loci (including 343617) are in Table 37. SYTX3486 (SEQ ID NO:1633) and SYTX6146 (SEQ ID NO:1634) show high values of Linkage Disequilibrium for both measurements, D′ and r², but all the other comparisons, show a large discrepancy between D′ and r². This is due to the presence of zeros in the table, or some cells with very small integers, and D′ becomes 1.0 as soon as there is a zero in the haplotype frequency table, whereas r² does not.

The SNP SYTX3486 and SYTX6146 show overall associations to NFI that are statistically significant, with SYTX3486 having P=0.00091. The favourable alleles for both SNP are at low frequency, f(0)=0.07 overall for both of them, with three breeds showing the favourable allele with a frequency of 0.01, although the Hereford breed has a frequency of approximately 0.35 for both of these SNP. The similarity in degree of significance for these SNP is consistent with the high LD values for both measures of LD. These SNP are one on either side of 343617, suggesting that the causative SNP has a low frequency of the favourable allele in this gene. A further SNP (SYTX10948—SEQ ID NO:1635) also shows association.

TABLE 37
Linkage disequilibrium measures between the 4 SYT10
SNP obtained by direct counting.

| Locus | Locus | h00 | h01 | h10 | h11 | f(a0) | f(b0) | D′ | r² |
|---|---|---|---|---|---|---|---|---|---|
| sytx3486 | sytx6146 | 44 | 1 | 1 | 2262 | 0.02 | 0.02 | 0.9773 | 0.9552 |
| sytx3486 | sytx10498 | 89 | 0 | 390 | 1909 | 0.04 | 0.20 | 1.0000 | 0.1543 |
| sytx3486 | sytx343617 | 0 | 139 | 549 | 1578 | 0.06 | 0.24 | 1.0000 | 0.0209 |
| sytx6146 | sytx10498 | 89 | 2 | 396 | 1907 | 0.04 | 0.20 | 0.9724 | 0.1471 |
| sytx6146 | sytx343617 | 1 | 142 | 547 | 1578 | 0.06 | 0.24 | 0.9711 | 0.0202 |
| sytx10498 | sytx343617 | 0 | 418 | 474 | 1230 | 0.20 | 0.22 | 1.0000 | 0.0706 |

The haplotypes are h00 to h11, the joint frequencies of the allele frequencies, the Lewontin D′ and the r² (Devlin and Risch, 1995) of each comparison is given.

### EXAMPLE 11

DNA samples for testing can be obtained from any tissue of the animal although for ease of use, tissues such as hair follicles, buccal swabs, ear punches or skin scrapings are the easiest. Blood samples are also easy to obtain, and for bulls that might be in progeny testing programs, semen samples are also an easy and convenient source of DNA. More generally, any non-cancerous tissue should give the same genotype, and this genotype can even be obtained form a fertilized egg or embryo in its earliest stages. Methods for extracting DNA from liquid or solid tissues have been reported in the literature and kits are available off the shelf to do so, and many methods have been customised to particular applications. Indeed, it is possible to obtain genotypes directly from a tissue without extensive purification, such as directly from micro-drops of blood, as would be known to a practitioner of the art.

Where DNA was extracted, it might be useful to know what the concentration of the DNA was and its purity, since some methods of genotyping are sensitive to the purity and the concentration of the DNA, not that the genotypes would be incorrect, but that the results might be so faint that the genotypes could not be scored. Nevertheless there are methods known in the art that are sufficiently robust that it would not be necessary to quantify the DNA before genotyping began.

Clearly, one might put in controls to ensure that the genotypes were accurate, although it would be possible to perform the genotyping without controls. Some controls would be negative in that they contained no DNA, and this would be useful to discriminate noise in the reactions from true genotypes. Some positive controls would also be useful, for animals of known genotype and of good quality DNA. In some cases DNA will not be of a high quality, and having some genotypes that are reliable will help in scoring genotypes since true exemplars can be referred to. In some cases, where a large number of samples were being genotyped, and the samples were located in 96-well or other format plates, it would be useful to have blank samples in known positions, in a different position in every plate, so that each plate would give a unique pattern of blanks. Then, if for some unforeseen reason an error occurred in labelling, this error could be detected because the blanks would not be where they should be.

Having set up the samples, genotyping could begin. Once an appropriate method of genotyping had been chosen, the results would be obtained. Most current high throughput methods involve electronic capture of genotyping signals and recording of the information on digital media, although some current methods still involve the recording of genotypes on analogue media, for example, by film photography or by recording the genotypes in note books.

One embodiment of the genotyping would be via the Taqman™ reaction although clearly this is not the only way genotypes could be collected. The DNA sequences listed in this specification would be subjected to analysis, where probes would be designed following the guidelines set out by Applied Biosystems (Foster City, Calif.) (LIVAK 2003). Not all DNA sequences are suitable for the Taqman™ reaction, and this can be determined by inspection or after subjecting the sequence to computerised analysis using software or the free service provided by the manufacturer. Nevertheless, 98% of sequences will be suitable, and a pair of probes, one each for the alternate alleles of the polymorphism, can be synthesized. DNA primers for the polymerase chain reaction will also be needed to amplify the DNA sequence around the polymorphism. The probes and primers are combined and then a polymerase chain reaction is performed on DNA of the animal, using standard methods following the manufacturers instructions. The genotypes are detected using a real time PCR machine and the raw data are collected after the samples are interrogated using laser fluorescence and digital image capture; the probes bind to DNA, and the polymerase collides with the probe, destroying it and releasing a dye which can then fluoresce; each probe is specific for an allele, each member of the pair has a different fluorescent molecule, and so homozygotes have only one of the two probes binding to the DNA while heterozygotes have both probes binding. So the fluorescent molecules report on which probe has been destroyed and hence the genotype of the individual, by the number and identity of fluorescent molecules that are released.

The genotypes can be used to predict the performance of the animal. This can be done simply by assigning a preferential ranking scheme for each animal based on the number of copies of the favourable alleles of the gene that it possesses. While this might be suitable for a small number of genes, it would be unwieldy for a large number of genes. Another way of implementing the DNA test results would be to give each animal the mean value for the trait, and then to add or subtract the average value of the genotype from the mean value. While this is not very accurate for one or a small number of polymorphisms, as the number of polymorphisms increases, the accuracy of prediction increases markedly. An advantage of such a method is that it allows the fitting of gene effects where the effects are not evenly spaced over the genotypes, for example, where one genotype might be quite different to the others. The most useful case is where the heterozygote shows overdominant effects, which do not fit easily into a scheme of additive rankings, that is, with one homozygote having low average values, the heterozygotes having intermediate average values, and the other homozygote having average high values, but rather where the heterozygotes easily outstrip both homozygotes in performance.

Once the animals have been assigned performance attributes for one or many traits depending upon which polymorphisms have been genotyped, these genotype combination and predicted attributes can be recorded, reported back to the owners, published or used to select animals for a variety of purposes. Some of the uses are 1) to breed animals with more or less desirable attributes for a particular trait or set of traits, and 2) to choose animals for particular purposes based upon a series of genotypes, for example, as a test on entry into a feedlot to select animals for particular market end points or customer requirements.

### REFERENCES

BARENDSE, W., and R. FRIES, 1999 Genetic linkage mapping, the gene maps of cattle, and the lists of loci, pp. 329-364 in The genetics of cattle, edited by R. FRIES and A. RUVINSKY. CABI Publishing, Wallingford.
CHEN, X. N., B. ZEHNBAUER, A. GNIRKE and P. Y. KWOK, 1997 Fluorescence energy transfer detection as a homogeneous DNA diagnostic method. Proceedings of the National Academy of Sciences of the United States of America 94: 10756-10761.
HARDENBOL, P., J. BANER, M. JAIN, M. NILSSON, E. A. NAMSARAEV et al., 2003 Multiplexed genotyping with sequence-tagged molecular inversion probes. Nature Biotechnology 21: 673-678.
HARDENBOL, P., F. L. YU, J. BELMONT, J. MACKENZIE, C. BRUCKNER et al., 2005 Highly multiplexed molecular inversion probe genotyping: Over 10,000 targeted SNPs genotyped in a single tube assay. Genome Research 15: 269-275.
LIVAK, K. J., 2003 SNP genotyping by the 5′-nuclease reaction, pp. 129-147 in Methods in Molecular Biology, edited by P.-Y. KWOK. Humana Press, Totowa N.J.
STORM, N., B. DARNHOFER-DEMAR, D. VAN DEN BOOM and C. P. RODI, 2002 MALDI-TOF mass spectrometry-based SNP genotyping. Methods in Molecular Biology 212: 214-262.
LIVAK, K. J., 2003 SNP genotyping by the 5′-nuclease reaction, pp. 129-147 in Methods in Molecular Biology, edited by P.-Y. KWOK. Humana Press, Totowa N.J.

1. A method for assessing a trait in a bovine animal selected from the group consisting of longissimus dorsi peak force, intramuscular fat, retail beef yield and net feed intake and/or its component traits, comprising the steps of:
(1) providing a nucleic acid from the bovine animal or carcass;
(2) assaying for the occurrence of a single nucleotide polymorphism (SNP) identified in any one of SEQ ID Nos: 1 to 1635, wherein the identification of said nucleotide occurrence as set forth (a) in any one of SEQ ID NOs: 1171 to 1631 is associated with variation in longissimus dorsi peak force, (b) in any of SEQ ID Nos: 214 to 842 is associated with intramuscular fat deposition, (c) in any one of SEQ ID NOs: 843 to 1170 is associated with retail beef yield and (d) in any one of SEQ ID NOs: 1 to 213 or 1632 to 1635 is associated with net feed intake and/or its component traits.

2. A method for selecting a bovine animal within a population of bovine animals, comprising the steps of:
(1) providing a nucleic acid sample from the bovine animal;
(2) assaying for the occurrence of a single nucleotide polymorphism (SNP) identified in any one of SEQ ID Nos: 1 to 1635, wherein the identification of said nucleotide occurrence as set forth (a) in any one of SEQ ID NOs: 1171 to 1631 is associated with variation in longissimus dorsi peak force, (b) in any of SEQ ID Nos: 214 to 842 is associated with intramuscular fat deposition, (c) in any one of SEQ ID NOs: 843 to 1170 is associated with retail beef yield and (d) in any one of SEQ ID NOs: 1 to 213 or 1632 to 1635 is associated with net feed intake and/or its component traits; and
(3) selecting a bovine animal exhibiting enhancement of the desired trait.

3. A method as claimed in either one of claims 1 or 2 wherein the nucleic acid is DNA.

4. A method as claimed in claim 3 wherein a DNA hybridization assay is used to detect the nucleotide occurrence.

5. A method as claimed in claim 3 wherein an amplification assay is used to detect the nucleotide occurrence.

6. A method as claimed in claim 5 wherein the amplification assay is PCR.

7. A method as claimed in claim 3 wherein the DNA comprises the sequence set forth in any one of SEQ ID Nos: 1 to 1635, or a fragment thereof of at least 10 contiguous nucleotides which contains the polymorphism.

8. A method as claimed in 1 or 2 wherein the bovine animal is a cow.

9. A method as claimed in claim 8 wherein the cow is a cross or pure breed selected from the group consisting of Angus, Ankole-Watusi, Ayrshire, Bazadaise, Beefalo, Beefmaster, Belgian Blue, Belmont Red, Blonde d'Aquitaine, Bonsmara, Braford, Brahman, Brahmousin, Brangus, Braunvieh, British White, American Brown Swiss, BueLingo, Charolais, Chianina, Corriente, American Devon, Dexters, Droughtmaster, Galloway, Gelbvieh, Guernsey, Hereford, Highland, Holstein, Jersey, Limousin, Lowline, Maine-Anjou, Marchigiana, Milking Shorthorn, Montebeliarde, Murray Grey, Normande, Parthenaise, Piedmontese, Pinzgauer, Romagnola, Salers, Salorn, Santa Gertrudis, Shetland, Shorthorn, Simmental, South Devon, Tarentaise, Texas Longhorn and Wagyu.

10. A method as claimed in claim 9 wherein the cow is a cross or pure breed selected from the group consisting of Angus, Shorthorn, Hereford, Murray Grey, Brahman, Belmont Red and Santa Gertrudis.

11. A solid substrate or surface comprising a plurality of nucleic acids in separate physical locations, including at least one nucleic acid as set forth in SEQ ID NOs: 1 to 1635, or a fragment thereof of at least 10 contiguous nucleotides which contain the polymorphism, immobilised thereon.

12. A kit for assessing a trait in a bovine animal selected from the group consisting of longissimus dorsi peak force, intramuscular fat, retail beef yield and net feed intake and/or its component traits through detection of the occurrence of a single nucleotide polymorphism (SNP) identified in any one of SEQ ID NOs: 1 to 1635, wherein the identification of said nucleotide occurrence as set forth (a) in any one of SEQ ID NOs: 1171 to 1631 is associated with variation in longissimus dorsi peak force, (b) in any of SEQ ID Nos: 214 to 842 is associated with intramuscular fat deposition, (c) in any one of SEQ ID NOs: 843 to 1170 is associated with retail beef yield and (d) in any one of SEQ ID NOs: 1 to 213 or 1632 to 1635 is associated with net feed intake and/or its component traits, comprising an oligonucleotide probe, primer or primer pair, or combinations thereof, for determining said nucleotide occurrence.

13. An oligonucleotide probe, primer or primer pair for detecting the occurrence of a single nucleotide polymorphism as set forth in any one of SEQ ID NOs: 1 to 1635.

14. A method for assessing a trait in a bovine animal selected from the group consisting of longissimus dorsi peak force, intramuscular fat, retail beef yield and net feed intake and/or its component traits, comprising the steps of:
(1) providing a nucleic acid from the bovine animal or carcass;
(2) assaying for the occurrence of a polymorphism in a gene, including in the coding sequences, the introns, promotors and other regulatory sequences of said gene, or a polymorphism in linkage disequilibrium with a polymorphism in said gene, wherein said gene is selected from the group consisting of CLCA4 to CLCA3, ABCC4, RPL11, TULP3, TRPC4, C6orf32, TPT1, GTF3C2, SLC6A15, CTNNA3, DDX46, HS2ST1, NDUFS3, PMS2, TMEM47, CEP1, CAP2, IMPG2, BAZ2B, SENP7, IMPG2, EFCBP2, DCP2, XBPP1, LAMA3, Synaptotagmin X (SYT 10), DMD, EMR2, ZNF33A, DNM1L, GBAS, SEC5L1, ATP1A1, YES1, BSN, POU4F1, SLIT3, ROBO1, KRT1-23, DDX10, GRM1 and BAAT, wherein the identification of said nucleotide occurrence (a) in any one of CLCA4 to CLCA3, ABCC4, RPL11, TULP3, TRPC4 and C6orf32 is associated with variation in longissimus dorsi peak force, (b) in any of TPT1, GTF3C2, SLC6A15, CTNNA3, DDX46, HS2ST1, NDUFS3, PMS2 and TMEM47 is associated with intramuscular fat deposition, (c) in any one of CEP1, CAP2, IMPG2, BAZ2B, SENP7, IMPG2, EFCBP2, DCP2, XBPP1 and LAMA3 is associated with retail beef yield and (d) in any one of Synaptotagmin X (SYT 10), DMD, EMR2, ZNF33A, DNM1L, GBAS, SEC5L1, ATP1A1, YES1, BSN, POU4F1, ROBO1, KRT1-23, DDX10 and BAAT is associated with net feed intake and/or its component traits.

15. A method for selecting a bovine animal within a population of bovine animals, comprising the steps of:
(1) providing a nucleic acid sample from the bovine animal;
(2) assaying for the occurrence of a polymorphism in a gene, including in the coding sequences, the introns, promotors and other regulatory sequences of said gene, or a polymorphism in linkage disequilibrium with a polymorphism in said gene, wherein said gene is selected from the group consisting of CLCA4 to CLCA3, ABCC4, RPL11, TULP3, TRPC4, C6orf32, TPT1, GTF3C2, SLC6A15, CTNNA3, DDX46, HS2ST1, NDUFS3, PMS2, TMEM47, CEP1, CAP2, IMPG2, BAZ2B, SENP7, IMPG2, EFCBP2, DCP2, XBPP1, LAMA3, Synaptotagmin X (SYT 10), DMD, EMR2, ZNF33A, DNM1L, GBAS, SEC5L1, ATP1A1, YES1, BSN, POU4F1, SLIT3, ROBO1, KRT1-23, DDX10, GRM1 and BAAT, and wherein the identification of said nucleotide occurrence (a) in any one of CLCA4 to CLCA3, ABCC4, RPL11, TULP3, TRPC4 and C6orf32 is associated with variation in longissimus dorsi peak force, (b) in any of TPT1, GTF3C2, SLC6A15, CTNNA3, DDX46, HS2ST1, NDUFS3, PMS2 and TMEM47 is associated with intramuscular fat deposition, (c) in any one of CEP1, CAP2, IMPG2, BAZ2B, SENP7, IMPG2, EFCBP2, DCP2, XBPP1 and LAMA3 is associated with retail beef yield and (d) in any one of Synaptotagmin X (SYT 10), DMD, EMR2, ZNF33A, DNM1L, GBAS, SEC5L1, ATP1A1, YES1, BSN, POU4F1, ROBO1, KRT1-23, DDX10 and BAAT is associated with net feed intake and/or its component traits; and
(3) selecting a bovine animal exhibiting enhancement of the desired trait.

16. A kit for assessing a trait in a bovine animal selected from the group consisting of longissimus dorsi peak force, intramuscular fat, retail beef yield and net feed intake and/or its component traits through detection of the occurrence of a single nucleotide polymorphism (SNP) in a gene, including in the coding sequences, the introns, promotors and other regulatory sequences of said gene, or a polymorphism in linkage disequilibrium with a polymorphism in said gene, wherein said gene is selected from the group consisting of CLCA4 to CLCA3, ABCC4, RPL11, TULP3, TRPC4, C6 orf2, TPT1, GTF3C2, SLC6A15, CTNNA3, DDX46, HS2ST1, NDUFS3, PMS2, TMEM47, CEP1, CAP2, IMPG2, BAZ2B, SENP7, IMPG2, EFCBP2, DCP2, XBPP1, LAMA3, Synaptotagmin X (SYT 10), DMD, EMR2, ZNF33A, DNM1L, GBAS, SEC5L1, ATP1A1, YES1, BSN, POU4F1, SLIT3, ROBO1, KRT1-23, DDX10, GRM1 and BAAT, and wherein the identification of said nucleotide occurrence (a) in any one of CLCA4 to CLCA3, ABCC4, RPL11, TULP3, TRPC4 and C6orf32 is associated with variation in longissimus dorsi peak force, (b) in any of TPT1, GTF3C2, SLC6A15, CTNNA3, DDX46, HS2ST1, NDUFS3, PMS2 and TMEM47 is associated with intramuscular fat deposition, (c) in any one of CEP1, CAP2, IMPG2, BAZ2B, SENP7, IMPG2, EFCBP2, DCP2, XBPP1 and LAMA3 is associated with retail beef yield and (d) in any one of Synaptotagmin X (SYT 10), DMD, EMR2, ZNF33A, DNM1L, GBAS, SEC5L1, ATP1A1, YES1, BSN, POU4F1, ROBO1, KRT1-23, DDX10 and BAAT is associated with net feed intake and/or its component traits, comprising an oligonucleotide probe, primer or primer pair, or combinations thereof, for determining said nucleotide occurrence.
